(19) United States
(12) Patent Application Publication
(10) Pub. No.: US 2009/0099157 A1
(43) Pub. Date: Apr. 16, 2009

(54) TETRAHYDRO-PYRAZOLO-PYRIDINE THIOETHER MODULATORS OF CATHEPSIN S

(76) Inventors: Michael K. Ameriks, San Diego, CA (US); Kristen L. Arienti, Le Mesa, CA (US); James P. Edwards, San Diego, CA (US); Cheryl A. Grice, Carlsbad, CA (US); Todd K. Jones, Solana Beach, CA (US); Alice Lee-Dutra, San Diego, CA (US); Jing Liu, San Diego, CA (US); Neelakandha S. Mani, San Diego, CA (US); Danielle K. Neff, La Jolla, CA (US); Alvah T. Wickboldt, San Diego, CA (US); John J.M. Wiener, La Jolla, CA (US)

Correspondence Address: WOODCOCK WASHBURN LLP, CIRA CENTRE, 12TH FLOOR, 2929 ARCH STREET, PHILADELPHIA, PA, 19104, US

(21) Appl. No.: 12/031,551

(22) Filed: Feb. 14, 2008

Related U.S. Application Data
(60) Provisional application No. 60/889,976, filed on Feb. 15, 2007.

(51) Int. Cl. A61K 31/551; C07D 471/04; A61K 31/5377; A61P 9/10; A61P 37/08; A61K 31/437

(52) U.S. Cl. 514/218; 546/119; 514/303; 540/575; 544/127; 514/234.2

(57) ABSTRACT

Tetrahydro-pyrazolo-pyridine thioether compounds are described, which are useful as cathepsin S modulators. Such compounds may be used in pharmaceutical compositions and methods for the treatment of disease states, disorders, and conditions mediated by cathepsin S activity, such as psoriasis, pain, multiple sclerosis, atherosclerosis, and rheumatoid arthritis.

This application claims the benefit of U.S. provisional patent application Ser. No. 60/889,976, filed Feb. 15, 2007 which is incorporated herein by reference.

### FIELD

The present invention relates to certain tetrahydro-pyrazolo-pyridine thioether compounds, pharmaceutical compositions containing them, and methods of using them for the treatment of disease states, disorders, and conditions mediated by cathepsin S activity.

### BACKGROUND

Cathepsin S is one of the major cysteine proteases expressed in the lysosome of antigen presenting cells, mainly dendritic cells, B cells and macrophages. Cathepsin S is best known for its critical function in the proteolytic digestion of the invariant chain chaperone molecules, thus controlling antigen presentation to CD4⁺ T cells by major histocompatibility complex class II molecules or to NK1.1⁺ T cells via CD1 molecules. Cathepsin S also appears to participate in direct processing of exogenous antigens for presentation by MHC class II to CD4⁺ T cells or crosspresentation by MHC class I molecules to CD8⁺ T cells. In addition, cathepsin S in secreted form is implicated in degradation of extracellular matrix, which may contribute to the pathology of a number of diseases, including arthritis, atherosclerosis, and chronic obstructive pulmonary disease. Therefore, inhibition of cathepsin S is a promising target for the development of novel therapeutics for a variety of indications. For a review, see: Thurmond, R. L. et al. Curr. Opin. Invest. Drugs 2005, 6(5), 473-482.

Pyrazole inhibitors of cathepsin S were disclosed in a series of applications from Ortho-McNeil, and additional publications on this work have also appeared (See: U.S. Pat. Appl. Publ. Nos. 2002/0040020, 2003/0078419, and 2002/0040019; Wei, J. et al. Bioorg. Med. Chem. Lett. 2007, 17, 5525-5528; Grice, C. A. et al. Bioorg. Med. Chem. Lett. 2006, 16, 2209-2212; and Gustin, D. J. et al. Bioorg. Med. Chem. Lett. 2005, 15, 1678-1691). See also: Thurmond, R. L. et al. J. Pharm. Exp. Ther. 2004, 308, 268-276; and Thurmond, R. L. et al. J. Med. Chem. 2004, 47, 4799-4801). However, there remains a need for potent cathepsin S modulators with desirable pharmaceutical properties.

### SUMMARY

In one aspect the invention relates to compounds of the following Formula (I):

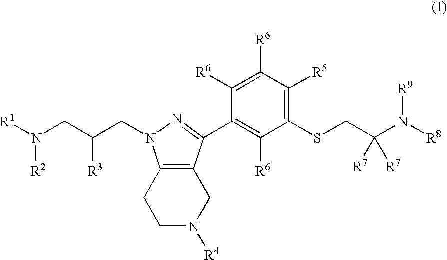

wherein:
R¹ and R² taken together with the nitrogen to which they are attached form a monocyclic heterocycloalkyl group, each group optionally containing one heteroatom ring member in addition to the nitrogen that is O, S, —N═, or NR^{a}, and each group unsubstituted or substituted with one, two, or three R^{b} substituents;
   where R^{a} is H, C₁₋₄alkyl, —COC₁₋₄alkyl, or —CO₂C₁₋₄alkyl, or a phenyl ring, monocyclic cycloalkyl ring, or monocyclic heteroaryl ring, each ring unsubstituted or substituted with OH, C₁₋₄alkyl, CF₃, halo, —OC₁₋₄alkyl, cyano, or —COC₁₋₄alkyl; and
   each R^{b} substituent is independently:
      i) OH, C₁₋₄alkyl, CF₃, —NR^{c}R^{d}, halo, —OC₁₋₄alkyl, CH₂F, CHF₂, —C₁₋₄alkyl-OH, —CH₂NR^{c}R^{d}, —COC₁₋₄alkyl, —CO₂C₁₋₄alkyl, —CO₂H, —CONR^{m}Rⁿ, CN, or —OCH₂CONR^{m}Rⁿ;
      ii) a monocyclic heterocycloalkyl group unsubstituted or substituted with C₁₋₄alkyl, OH, —OC₁₋₄alkyl, —NR^{c}R^{d}, or halo;
      iii) a monocyclic heterocycloalkyl group fused with a phenyl or pyridyl group, the resulting fused bicyclic group being unsubstituted or substituted with C₁₋₄alkyl, OH, —OC₁₋₄alkyl, —NR^{c}R^{d}, or halo; or
      iv) a phenyl group, benzyl group, monocyclic heteroaryl group, monocyclic cycloalkyl group, —O-phenyl group, or —O-(monocyclic heteroaryl) group, each group unsubstituted or substituted with C₁₋₄alkyl, OH, —OC₁₋₄alkyl, CF₃, cyano, —NR^{c}R^{d}, benzyl or halo; or
      v) two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, —NR^{c}R^{d}, or halo; or
      vi) two R^{b} substituents on adjacent carbons taken together with the carbons to which they are attached form a saturated monocyclic cycloalkyl, phenyl, or monocyclic heteroaryl group, each group unsubstituted or substituted with C₁₋₄alkyl, OH, —OC₁₋₄alkyl, —NR^{c}R^{d}, or halo;
      where R^{c} and R^{d} are each independently H, C₁₋₄alkyl, —COC₁₋₄alkyl, —COC₁₋₄alkyl-OH, —CO₂C₁₋₄alkyl, —CONR^{x}R^{y}, —C₁₋₄alkyl-OH, or —SO₂C₁₋₄alkyl;
         where R^{x} and R^{y} are each independently H or C₁₋₄alkyl; and
      R^{m} and Rⁿ are each independently H or C₁₋₄alkyl; or R^{m} and Rⁿ taken together with the nitrogen to which they are attached form a monocyclic heterocycloalkyl ring;
R³ is H, OH, C₁₋₄alkyl, or —OC₁₋₄alkyl;
R⁴ is H; C₁₋₄alkyl; —COC₁₋₄alkyl unsubstituted or substituted with OH, F, —OCOC₁₋₄alkyl, —OC₁₋₄alkyl, phenyl, —SC₁₋₄alkyl, —NR^{o}R^{p}, or —N(R^{o})CO₂C₁₋₄alkyl; —CO-(monocyclic cycloalkyl); —CO-(monocyclic heteroaryl); —CO—(C-linked monocyclic heterocycloalkyl); —COCH₂— (monocyclic heterocycloalkyl); —SO₂C₁₋₄alkyl; —SO₂CF₃; —SO₂—(monocyclic cycloalkyl); —SO₂-phenyl; —SO₂-benzyl; —SO₂—NR^{o}R^{p}; —CONR^{o}R^{p}; —COCO₂C₁₋₄alkyl; —COCONH₂; or —COCONHC₁₋₄alkyl; wherein each cycloalkyl, heteroaryl, heterocycloalkyl, phenyl, or benzyl group is unsubstituted or substituted with C₁₋₄alkyl, OH, F, —COC₁₋₄alkyl, —COCF₃, —CO₂C₁₋₄alkyl, —N(R^{o})CO₂C₁₋₄alkyl, or —NR^{o}R^{p};
   where R^{o} and R^{p} are each independently H or C₁₋₄alkyl, or a monocyclic cycloalkyl, phenyl, or monocyclic heteroaryl group; or R^{o} and R^{p} taken together with the nitrogen to which they are attached form a monocyclic heterocycloalkyl group; wherein each cycloalkyl, phenyl, heteroaryl, or heterocycloalkyl group is unsubstituted or substituted with C₁₋₄alkyl, OH, —OC₁₋₄alkyl, —NR^{s}R^{t}, or halo;
      where R^{s} and R^{t} are each independently H, C₁₋₄alkyl, —COC₁₋₄alkyl, —CO₂C₁₋₄alkyl, —CONR^{v}R^{w}, C₁₋₄alkyl-OH, or —SO₂C₁₋₄alkyl;
         where R^{v} and R^{w} are each independently H or C₁₋₄alkyl;
R⁵ is halo or CF₃;
each R⁶ is independently H or F;
each R⁷ is H or both R⁷ together form a carbonyl;
R⁸ is H or C₁₋₄alkyl and R⁹ is R¹⁰, —COR¹¹, —SO₂R¹¹, —SO₂CF₃, —SO₂N(C₁₋₄alkyl)₂, —CO₂C₁₋₄alkyl, or —CO₂-benzyl;
   where R¹⁰ and R¹¹ are each: a C₁₋₄alkyl group unsubstituted or substituted with R^{e}; or a monocyclic cycloalkyl group, bicyclic cycloalkyl group, monocyclic heterocycloalkyl group, phenyl group, or fluorenyl group, each group unsubstituted or substituted with C₁₋₄alkyl, OH, —OC₁₋₄alkyl, —NR^{f}R^{g}, halo, —COC₁₋₄alkyl, —CO₂C₁₋₄alkyl, phenyl, benzyl, or benzyloxy;
      where R^{e} is OH, —OC₁₋₄alkyl, or NR^{f}R^{g}, or a monocyclic cycloalkyl group, monocyclic heterocycloalkyl group, monocyclic heteroaryl group, phenyl group, or —O-phenyl group, each group unsubstituted or substituted with C₁₋₄alkyl, OH, —OC₁₋₄alkyl, —NR^{f}R^{g}, halo, —COC₁₋₄alkyl, or —CO₂C₁₋₄alkyl;
      where R^{f} and R^{g} are each independently H, C₁₋₄alkyl, —COC₁₋₄alkyl, or —CO₂C₁₋₄alkyl;
or R⁸ and R⁹ taken together with the nitrogen to which they are attached form a monocyclic heterocycloalkyl group, each group optionally containing one heteroatom ring member in addition to the nitrogen that is O, S, SO₂, —N═, or NR^{h}, and each group unsubstituted or substituted with one or two Rⁱ substituents;
   where R^{h} is H, C₁₋₄alkyl, phenyl, —C₁₋₂alkyl-phenyl, —COC₁₋₄alkyl, or —CO₂C₁₋₄alkyl; and
   each Rⁱ substituent is independently: a C₁₋₄alkyl group unsubstituted or substituted with OH or phenyl; CF₃; halo; OH; —OC₁₋₄alkyl; —NR^{j}R^{k}; —COC₁₋₄alkyl; —CO₂C₁₋₄alkyl; —CONR^{j}R^{k}; phenyl; pyridyl; or benzyl; or two adjacent Rⁱ substituents taken together with the carbons to which they are attached form a phenyl or monocyclic cycloalkyl group;
      where R^{j} and R^{k} are each independently H, C₁₋₄alkyl, —COC₁₋₄alkyl, —COC₁₋₄alkyl-OH, or —CO₂C₁₋₄alkyl; or R^{j} and R^{k} taken together with the nitrogen to which they are attached form a monocyclic saturated heterocycloalkyl ring;
         and pharmaceutically acceptable salts, prodrugs, and metabolites thereof.

In certain embodiments, the compound of Formula (I) is a compound selected from those species described or exemplified in the detailed description below.

In a further aspect, the invention relates to pharmaceutical compositions each comprising: (a) an effective amount of at least one chemical entity selected from compounds of Formula (I), and pharmaceutically acceptable salts, prodrugs, and metabolites thereof; and (b) a pharmaceutically acceptable excipient.

In another aspect, the invention is directed to a method of treating a subject suffering from or diagnosed with a disease, disorder, or medical condition mediated by cathepsin S activity, comprising administering to the subject in need of such treatment an effective amount of at least one chemical entity selected from compounds of Formula (I), and pharmaceutically acceptable salts, prodrugs, and metabolites thereof. Diseases, disorders and medical conditions that are mediated by cathepsin S activity include those referred to herein.

In another aspect, the chemical entities of the present invention are useful as cathepsin S modulators. Thus, the invention is directed to a method for modulating cathepsin S activity, including when such receptor is in a subject, comprising exposing cathepsin S to an effective amount of at least one chemical entity selected from compounds of Formula (I), pharmaceutically acceptable salts of compounds of Formula (I), pharmaceutically acceptable prodrugs of compounds of Formula (I), and pharmaceutically active metabolites of compounds of Formula (I).

Additional embodiments, features, and advantages of the invention will be apparent from the following detailed description and through practice of the invention.

### DETAILED DESCRIPTION

For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a saturated, straight- or branched-chain alkyl group having from 1 to 12 carbon atoms in the chain. Examples of alkyl groups include methyl (Me, which also may be structurally depicted by a bond, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

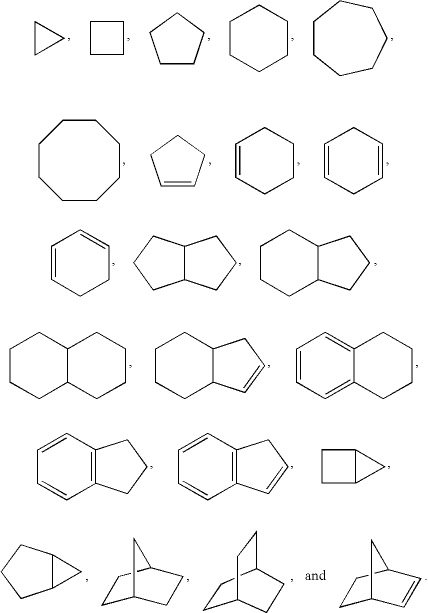

A “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. In preferred embodiments, heterocycloalkyl rings have one or two heteroatoms. The ring structure may optionally contain up to two oxo groups on carbon or sulfur ring members. Illustrative entities, in the form of properly bonded moieties, include:

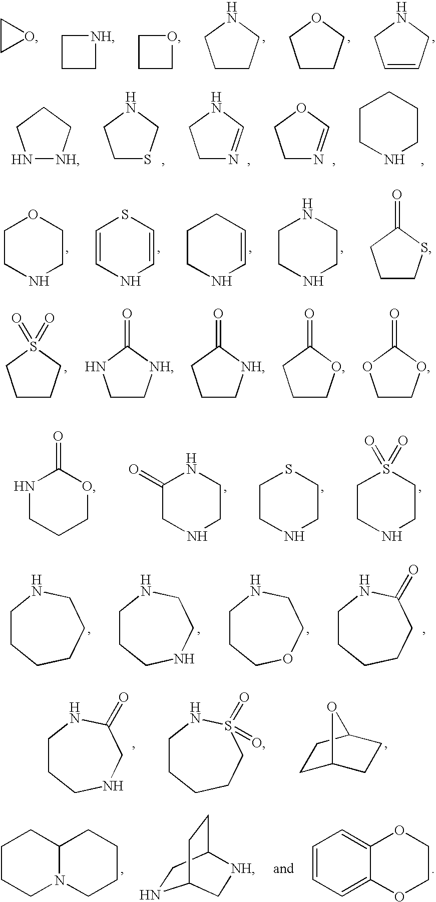

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

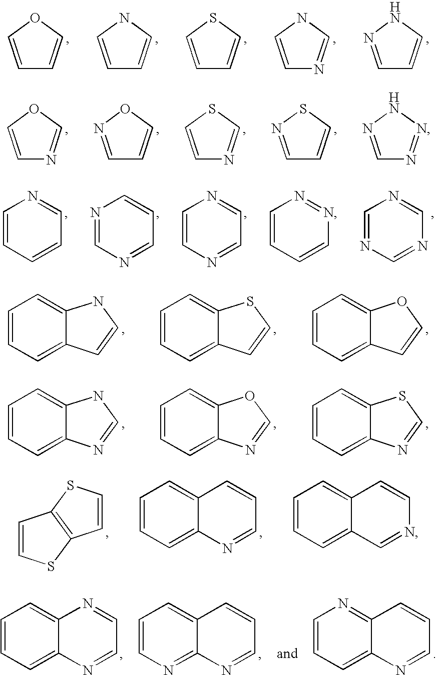

Those skilled in the art will recognize that the species of heteroaryl, cycloalkyl, and heterocycloalkyl groups listed or illustrated above are not exhaustive, and that additional species within the scope of these defined terms may also be selected.

The term “halogen” represents chlorine, fluorine, bromine, or iodine. The term “halo” represents chloro, fluoro, bromo, or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system that yields a stable chemical structure.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to represent hydrates, solvates, and polymorphs of such compounds, and mixtures thereof.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Reference to a chemical entity herein stands for a reference to any one of: (a) the actually recited form of such chemical entity, and (b) any of the forms of such chemical entity in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, salvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³²P, ³³P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to any generic substituent symbol used herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to any generic substituent symbol used herein.

The nomenclature “Cᵢ₋ⱼ” with j>i, when applied herein to a class of substituents, is meant to refer to embodiments of this invention for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃).

The term Cₙ₋ₘalkyl refers to an aliphatic chain, whether straight or branched, with a total number N of carbon members in the chain that satisfies n≦N≦m, with m>n.

Any disubstituent referred to herein is meant to encompass the various attachment possibilities when more than one of such possibilities are allowed. For example, reference to disubstituent -A-B-, where A≠B, refers herein to such disubstituent with A attached to a first substituted member and B attached to a second substituted member, and it also refers to such disubstituent with A attached to the second substituted member and B attached to the first substituted member.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

In some embodiments of Formula (I), —NR¹R² is a structure of Formula (II):

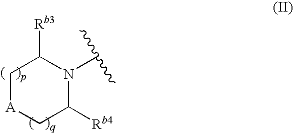

wherein:
A is NR^{a}, O, S, or C(R^{b1})(R^{b2});
   where R^{a} is methyl, acetyl, or cyclopropyl; or a phenyl or pyridyl ring, each ring unsubstituted or substituted with OH, halo, —OC₁₋₄alkyl, cyano, or acetyl;
   R^{b1} is H or methyl; and
   R^{b2} is H, methyl, or OH; or a monocyclic heterocycloalkyl group unsubstituted or substituted with methyl, OH, or halo; or a monocyclic heterocycloalkyl group fused with a phenyl or pyridyl group, the resulting fused bicyclic group being unsubstituted or substituted with methyl, OH, dimethylamino, or halo; or a phenyl or pyridyl ring unsubstituted or substituted with methyl or halo;
one of R^{b3} and R^{b4} is H and the other is H or C₁₋₄alkyl;
p is 0, 1, or 2; and
q is 0, 1, 2, or 3;
with the proviso that when A is NR^{a}, O, S, or SO₂, then p and q are each greater than or equal to 1.

In other embodiments of Formula (I), R¹ and R² taken together with the nitrogen to which they are attached form azetidine, pyrrolidine, piperidine, piperazine substituted with R^{a}, morpholine, or thiomorpholine, each unsubstituted or substituted with one, two, or three R^{b} substituents. In other embodiments, R¹ and R² taken together with the nitrogen to which they are attached form [1,4]diazepane substituted with R^{a}, azepane, 1,4,5,6-tetrahydro-pyrimidine substituted with R^{a}, 1,2,3,6-tetrahydropyridine, or piperazinone substituted with R^{a}, each unsubstituted or substituted with one, two, or three R^{b} substituents. In other embodiments, R¹ and R² taken together with the nitrogen to which they are attached form piperidine or piperazine substituted with R^{a}, each unsubstituted or substituted with one, two, or three R^{b} substituents.

In some embodiments, R^{a} is H, methyl, isopropyl, acetyl, or tertbutoxycarbonyl. In other embodiments, R^{a} is phenyl, cyclopropyl, pyridinyl, pyrimidinyl, or pyrazinyl, each optionally substituted. In still other embodiments, R^{a} is pyrimidin-2-yl, pyrazinyl, phenyl, 2-hydroxyphenyl, 3-hydroxyphenyl, 4-hydroxyphenyl, 2-fluorophenyl, 4-fluorophenyl, 2-cyanophenyl, 4-cyanophenyl, 2-methoxyphenyl, 3-methoxyphenyl, 4-methoxyphenyl, 2-methylphenyl, 2-ethoxyphenyl, 4-acetylphenyl, 2-chlorophenyl, 3-trifluoromethylphenyl, 3-chloropyridin-2-yl, or 3-cyano-pyridin-2-yl.

In some embodiments, each R^{b} substituent is independently OH, methyl, CF₃, dimethylamino, acetamido, tert-butoxycarbamoyl, fluoro, or methoxy. In other embodiments, each R^{b} substituent is independently carbamoyl, tert-butyl, 1-hydroxy-1-methyl-ethyl, ureido, 2-hydroxy-acetylamino, ethoxycarbamoyl, methanesulfonylamino, pyrrolidine-1-carbonyl, amino, morpholine-4-carbonyl, dimethylaminomethyl, hydroxymethyl, acetylamino-methyl, carboxy, hydroxyethyl, difluoromethyl, piperidine-1-carbonyl, ethanesulfonylamino, 2-hydroxy-propionylamino, cyano, 2-hydroxy-2-methyl-propionylamino, or 2-oxo-2-pyrrolidin-1-yl-ethoxy. In other embodiments, R^{b} is pyrrolidinyl, 2-oxo-pyrrolidinyl, or piperidinyl, each optionally substituted. In still other embodiments, R^{b} is pyrrolidin-1-yl, 2-oxo-pyrrolidin-1-yl, 5-dimethylamino-1-methyl-1,3-dihydro-imidazo[4,5-b]pyridin-2-onyl, or 4-4H-benzo[1,4]oxazin-3-onyl. In still other embodiments, R^{b} is 5-oxo-4,5-dihydro-1H-[1,2,4]triazol-3-yl, morpholin-1-yl, pyrrolidin-2-on-1-yl, 4-hydroxy-2-oxo-pyrrolidin-1-yl, piperidin-1-yl, 5-oxo-1,5-dihydro-[1,2,4]triazol-4-yl, 5-chloro-1-methyl-1,3-dihydro-2H-benzimidazol-2-on-1-yl, or 5-chloro-1,3-dihydro-2H-benzimidazol-2-on-1-yl. In other embodiments, R^{b} is phenyl, pyridyl, pyrimidinyl, oxadiazolyl, 1H-pyrrolo[2,3-b]pyridin-3-yl, tetrazolyl, pyrimidinyloxy, pyridinyloxy, cyclohexyl, or benzyl, each optionally substituted. In further embodiments, R^{b} is phenyl, 4-cyanophenyl, 3-cyanophenyl, 2-methoxyphenyl, 4-methoxyphenyl, 3-chlorophenyl, 3-trifluoromethylphenyl, 2-pyridinyl, 3-pyridinyl, 4-pyridinyl, 2-pyrimidinyl, 3-methyl-[1,2,4]oxadiazol-5-yl, 3-chloro-pyridin-2-yl, 1H-pyrrolo[2,3-b]pyridin-3-yl, 3,5-dichloro-pyridin-4-yl, 1-benzyl-1H-tetrazol-5-yl, cyclohexyl, benzyl, hydroxytriazolyl, pyrimidin-2-yloxy, or pyridin-2-yloxy. In other embodiments, two R^{b} substituents at the same carbon taken together with the carbon to which they are attached form a [1,3]dioxolane or [1,3]dioxane ring, each optionally substituted. In other embodiments, two R^{b} substituents on adjacent carbons taken together with the monocyclic heterocycloalkyl group to which they are attached form a fused ring system selected from the group consisting of hexahydro-cyclopenta[c]pyrrol-2-yl, 1,3-dihydro-isoindol-2-yl, 2,4,5,7-tetrahydro-pyrazolo[3,4-c]pyridin-6-yl, 1,4,6,7-tetrahydro-imidazo[4,5-c]pyridin-5-yl, 3,4-dihydro-1H-isoquinolin-2-yl, or octahydro-isoquinolin-2-yl, each optionally substituted.

In some embodiments, R³ is H or OH.

In some embodiments, R⁴ is H, methyl, ethyl, acetyl, 2-hydroxyacetyl, 2-methoxyacetyl, 2-acetoxyacetyl, propionyl, 2-hydroxy-propionyl, 2-hydroxy-2-methylpropionyl, 2-hydroxy-2-phenyl-acetyl, 2-aminoacetyl, 2-dimethylamino-acetyl, 2-tert-butoxycarbonylamino-acetyl, 2-methylsulfanyl-acetyl, 2-(piperidin-4-yl)-acetyl, 2-(1-tert-butoxycarbonyl-piperidin-4-yl)-acetyl, or methoxyoxalyl. In other embodiments, R⁴ is aminooxalyl, methylaminooxalyl, carbamoyl, methylcarbamoyl, dimethylcarbamoyl, isopropylcarbamoyl, sec-butylcarbamoyl, tert-butylcarbamoyl, diethylcarbamoyl, diisopropylcarbamoyl, pyrrolidine-1-carbonyl, morpholine-4-carbonyl, phenylcarbamoyl, cyclohexylcarbamoyl, thiophen-2-ylcarbamoyl, 4-methyl-piperazine-1-carbonyl, or (4-dimethylaminophenyl)carbamoyl. In other embodiments, R⁴ is ethanesulfonyl, propanesulfonyl, isopropanesulfonyl, trifluoromethanesulfonyl, cyclopropanesulfonyl, benzenesulfonyl, benzylsulfonyl, or dimethylsulfamoyl. In other embodiments, R⁴ is cyclobutanecarbonyl, 1-amino-cyclopropanecarbonyl, 1-tert-butoxycarbonylamino-cyclopropanecarbonyl, 1H-[1,2,4]triazole-3-carbonyl, 1-methyl-1H-imidazole-2-carbonyl, thiophene-2-carbonyl, furan-2-carbonyl, tetrahydrofuran-2-carbonyl, 1-methylpyrrolidine-2-carbonyl, 1-acetyl-pyrrolidine-2-carbonyl, 1-trifluoroacetyl-pyrrolidine-2-carbonyl, piperidine-3-carbonyl, or 1-tert-butoxycarbonyl-piperidine-3-carbonyl. In other embodiments, R⁴ is —SO₂CH₃, —CONH₂, or —COCONH₂. In other embodiments, R⁴ is —SO₂CH₃.

In some embodiments, R⁵ is chloro or CF₃. In other embodiments, R⁵ is CF₃.

In some embodiments, each R⁶ is H.

In some embodiments, each R⁷ is H. In other embodiments, both R⁷ together form a carbonyl.

In some embodiments, R⁸ is H or methyl.

In some embodiments, R⁹ is a methyl, ethyl, propyl, isopropyl, sec-butyl, iso-butyl, or tert-butyl group, each unsubstituted or substituted with R^{e}. In other embodiments, R⁹ is R¹⁰, and R¹⁰ is selected from the group consisting of cyclopropyl, cyclobutyl, cyclopentyl, cyclohexyl, indanyl, and piperidin-4-yl, each unsubstituted or substituted with methyl, OH, methoxy, dimethylamino, tert-butoxycarbonyl, or halo. In other embodiments, R¹⁰ is a phenyl group, unsubstituted or substituted with methyl, OH, halo, dimethylamino, phenyl, or benzyloxy. In other embodiments, R¹⁰ is a benzyl or phenethyl group.

In some embodiments, R⁹ is —COR¹¹, and R¹¹ is a methyl, ethyl, or propyl group, each unsubstituted or substituted with R^{e}. In other embodiments, R¹¹ is selected from the group consisting of cyclopropyl, cyclobutyl, cyclopentyl, cyclohexyl, indanyl, and piperidin-4-yl, each unsubstituted or substituted with methyl, OH, methoxy, or halo. In other embodiments, R¹¹ is a phenyl group, unsubstituted or substituted with methyl, ethyl, OH, halo, dimethylamino, or tert-butoxycarbonyl.

In some embodiments, R^{e} is OH, methoxy, dimethylamino, diethylamino, or tert-butoxycarbamoyl. In other embodiments, R^{e} is a cyclopropyl, cyclopentyl, cyclohexyl, furanyl, thiophenyl, pyridyl, phenyl, phenoxy, or imidazolyl group, each unsubstituted or substituted with methyl, OH, methoxy, dimethylamino, or halo. In still other embodiments, R^{e} is OH, methoxy, thiophen-2-yl, cyclohexenyl, phenoxy, piperidinyl, pyrrolidinyl, or tert-butoxycarbamoyl.

In some embodiments, R⁹ is tert-butoxycarbonyl, cyclopentyl, cyclohexyl, benzyl, methyl, 3-methoxypropyl, thiophen-2-ylmethyl, 2-cyclohex-1-enyl-ethyl, 2-phenoxy-ethyl, 2-morpholin-4-yl-ethyl, 1-ethyl-pyrrolidin-2-ylmethyl, 4-biphenyl, indan-2-yl, 3-biphenyl, 9H-fluoren-1-yl, 4-benzyloxy-phenyl, phenethyl, 4-fluorophenyl, or 1-methyl-piperidin-4-yl. In other embodiments, R⁹ is propyl, isopropyl, isobutyl, cyclopropyl, 1-methyl-3-phenyl-propyl, 3-hydroxy-propyl, 2-(1H-imidazol-4-yl)-ethyl, or 1-(tert-butoxycarbonyl)-piperidin-4-yl. In other embodiments, R⁹ is benzoyl, phenylacetyl, 1-tert-butoxycarbonyl-piperidine-4-carbonyl, tert-butylcarbamoyl-acetyl, 4-fluorobenzoyl, 4-hydroxybenzoyl, 4-dimethylamino-benzoyl, 4-fluorophenylacetyl, 1-tert-butoxycarbonyl-piperidine-3-carbonyl, 1-tert-butoxycarbonyl-pyrrolidine-3-carbonyl, 4-tert-butylcarbamoyl-1-butyryl, piperidine-3-carbonyl, pyrrolidine-3-carbonyl, piperidine-4-carbonyl, or benzyloxycarbonyl.

In some embodiments, R⁹ is —SO₂R¹¹, —SO₂CF₃, or —SO₂N(C₁₋₄alkyl)₂. In further embodiments, R⁹ is methanesulfonyl, ethanesulfonyl, dimethylsulfamoyl, trifluoromethanesulfonyl, or benzenesulfonyl.

In some embodiments, R⁸ and R⁹ taken together with the nitrogen to which they are attached form azetidine, pyrrolidine, piperidine, piperazine substituted with R^{h}, morpholine, thiomorpholine, 1,1-dioxo-1λ⁶-thiomorpholine, azepine, diazepine substituted with R^{h}, 3,6-dihydro-2H-pyridine, or thiazolidine, each unsubstituted or substituted with Rⁱ as described for Formula (I). In other embodiments, R⁸ and R⁹ taken together with the nitrogen to which they are attached form pyrrolidine or piperidine, each unsubstituted or substituted with R^{h} and Rⁱ as described for Formula (I). In other embodiments, R⁸ and R⁹ taken together with the nitrogen to which they are attached form piperazine, morpholine, azetidine, oxo-imidazolidin-1-yl, dihydro-pyrimidin-1-yl, or oxo-piperazinyl.

In some embodiments, R^{h} is H, methyl, isopropyl, acetyl, tertbutoxycarbonyl, phenyl, or phenethyl.

In some embodiments, each Rⁱ substituent is independently: a methyl or ethyl group unsubstituted or substituted with OH or phenyl; tert-butyl; CF₃; fluoro; OH; methoxy; dimethylamino; acetylamino; 2-hydroxy-acetylamino; ethoxycarbonylamino; tert-butoxycarbonylamino; piperidin-1-yl; pyrrolidin-1-yl; acetyl; methoxycarbonyl; carbamoyl; dimethylcarbamoyl; diethylcarbamoyl; phenyl; pyridyl; or benzyl; or two adjacent Rⁱ substituents taken together with the carbons to which they are attached form phenyl, cyclopentyl, or cyclohexyl.

In other embodiments, R⁸ and R⁹ taken together with the nitrogen to which they are attached form piperidin-1-yl, 4-hydroxy-piperidin-1-yl, octahydro-isoquinolin-2-yl, 4,4-difluoro-piperidin-1-yl, 3-methyl-piperidin-1-yl, 3,5-dimethylpiperidin-1-yl, 4-methyl-piperidin-1-yl, 3,3-difluoro-piperidin-1-yl, octahydro-quinolin-1-yl, 3-hydroxymethyl-piperidin-1-yl, 3,6-dihydro-2H-pyridin-1-yl, 1,1-dioxo-1λ⁶-thiomorpholin-4-yl, 4-tert-butyl-piperidin-1-yl, 3-fluoro-piperidin-1-yl, 2-methyl-piperidin-1-yl, 4-phenyl-piperidin-1-yl, 2-trifluoromethyl-pyrrolidin-1-yl, morpholin-1-yl, 4-(2-hydroxy-ethyl)-piperidin-1-yl, pyrrolidin-1-yl, 4-phenyl-piperazin-1-yl, 2-methyl-pyrrolidin-1-yl, 4-methylpiperazin-1-yl, 4-dimethylamino-piperidin-1-yl, 2-phenyl-piperidin-1-yl, 4-carbamoyl-piperidin-1-yl, 4-methoxy-piperidin-1-yl, 3-trifluoromethyl-piperidin-1-yl, 2-hydroxymethyl-piperidin-1-yl, 4-trifluoromethyl-piperidin-1-yl, 2-thiazolidin-3-yl, 3-hydroxy-pyrrolidin-1-yl, 3,4-dihydro-1H-isoquinolin-2-yl, 4-methyl-[1,4]diazepan-1-yl, 3-diethylcarbamoyl-piperidin-1-yl, 3-dimethylamino-pyrrolidin-1-yl, 4-phenyl-3,6-dihydro-2H-pyridin-1-yl, [1,4′]bipiperidinyl-1′-yl, 2-azepan-1-yl, 4-benzyl-piperidin-1-yl, 4-phenethyl-piperidin-1-yl, 4-diethylcarbamoyl-piperidin-1-yl, 2-carbamoyl-pyrrolidin-1-yl, 4-fluoro-piperidin-1-yl, 4-isopropyl-piperazin-1-yl, or 4-phenethyl-piperazin-1-yl. In other embodiments, R⁸ and R⁹ taken together with the nitrogen to which they are attached form 3-hydroxy-piperidin-1-yl, 4-(acetylamino)-piperidin-1-yl, 3-hydroxymethyl-piperidin-1-yl, 4-(pyrrolidin-1-yl)-piperidin-1-yl, 4-(1-hydroxy-1-methyl-ethyl)-piperidin-1-yl, 4-(2-hydroxy-acetylamino)-piperidin-1-yl, 4-hydroxy-4-(2-pyridinyl)-piperidin-1-yl, 3,3-dimethyl-piperidin-1-yl, 3-phenyl-piperidin-1-yl, 4-ethoxycarbonylamino-piperidin-1-yl, 2-benzyl-piperidin-1-yl, 3-benzyl-piperidin-1-yl, 4-tert-butoxycarbonylamino-piperidin-1-yl, 4-acetyl-piperazin-1-yl, 3-methyl-morpholin-4-yl, 2,6-dimethyl-morpholin-4-yl, 3-hydroxymethyl-morpholin-4-yl, 2-hydroxylmethyl-morpholin-4-yl, 3,3-difluoro-pyrrolidin-1-yl, 3-fluoro-pyrrolidin-1-yl, 3-hydroxymethyl-pyrrolidin-1-yl, azetidinyl, 4-oxo-imidazolidin-1-yl, 5,6-dihydro-4H-pyrimidin-1-yl, 2-methyl-3-oxo-piperazin-1-yl), or hexahydro-cyclopenta[c]pyrrol-2-yl.

In other embodiments, compounds of Formula (I) are selected from compounds of the following Formula (IV):

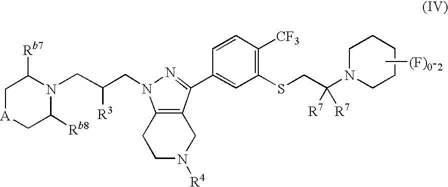

A is NR^{a}, O, S, or C(R^{b5})(R^{b6});
   where R^{a} is methyl, acetyl, or cyclopropyl; or a phenyl or pyridyl ring, each ring unsubstituted or substituted with OH, halo, —OC₁₋₄alkyl, cyano, or acetyl;
   R^{b5} is H or methyl; and
   R^{b6} is H, methyl, or OH; or a monocyclic heterocycloalkyl group unsubstituted or substituted with methyl, OH, or halo; or a monocyclic heterocycloalkyl group fused with a phenyl or pyridyl group, the resulting fused bicyclic group being unsubstituted or substituted with methyl, OH, dimethylamino, or halo; or a phenyl or pyridyl ring unsubstituted or substituted with methyl or halo;
one of R^{b7} and R^{b8} is H and the other is H or C₁₋₄alkyl;
R³ is H or OH;
R⁴ is —SO₂CH₃, —CONH₂, or —COCONH₂; and
each R⁷ is H or both R⁷ together form a carbonyl;
   and pharmaceutically acceptable salts, prodrugs, and metabolites thereof.

Other embodiments include combinations of the above variable definitions.

The invention includes also pharmaceutically acceptable salts of the compounds represented by Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), pharmaceutical compositions containing such pharmaceutically acceptable prodrugs, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I), and uses of such metabolites in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J. Med. Chem. 1997, 40, 2011-2016; Shan, et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites (collectively, “active agents”) of the present invention are useful in the methods of the invention.

The compounds of Formula (I) and their pharmaceutically acceptable salts, pharmaceutically acceptable prodrugs, and pharmaceutically active metabolites, whether alone or in combination, (collectively, “active agents”) of the present invention are useful as cathepsin S modulators in the methods of the invention. Such methods for modulating cathepsin S activity comprise exposing cathepsin S to an effective amount of at least one chemical entity selected from compounds of Formula (I), pharmaceutically acceptable salts of compounds of Formula (I), pharmaceutically acceptable prodrugs of compounds of Formula (I), and pharmaceutically active metabolites of compounds of Formula (I). Embodiments of this invention inhibit cathepsin S activity.

In some embodiments, the cathepsin S is in a subject with a disease, disorder, or medical condition mediated through modulation of the cathepsin S, such as those described herein. Symptoms or disease states are intended to be included within the scope of “medical conditions, disorders, or diseases.”

Accordingly, the invention relates to methods of using the active agents described herein to treat subjects diagnosed with or suffering from a disease, disorder, or condition mediated through cathepsin S activity, such as an autoimmune disease, an allergic condition, inflammation, a bowel disorder, tissue transplant rejection, pain, neuropathic pain, or cancer. Active agents according to the invention may therefore be used as immunomodulating agents, immunosuppressants, anti-allergy agents, anti-inflammatory agents, analgesics, or anti-cancer agents.

In some embodiments, an active agent of the present invention is administered to treat lupus, asthma, allergic reaction, atopic allergy, hay fever, atopic dermatitis, food allergy, rhinitis (such as allergic rhinitis and the inflammation caused by non-allergic rhinitis), skin immune system disorders (such as psoriasis), uveitis, inflammation, upper airway inflammation, Sjögren's syndrome, arthritis, rheumatoid arthritis, osteoarthritis, type I diabetes, atherosclerosis, multiple sclerosis, coeliac disease, inflammatory bowel disease (IBD), chronic obstructive pulmonary disorder (COPD), tissue transplant rejection, pain, chronic pain (such as pain due to conditions such as cancer, neuropathic pain, rheumatoid arthritis, osteoarthritis and inflammatory conditions), or cancer (and cancer-related processes such as angiogenesis, tumor growth, cell proliferation, and metastasis). In certain embodiments, an active agent of the present invention is administered to treat psoriasis, pain, multiple sclerosis, atherosclerosis, or rheumatoid arthritis.

Thus, the active agents may be used to treat subjects diagnosed with or suffering from a disease, disorder, or condition mediated through cathepsin S activity. The term “treat” or “treating” as used herein is intended to refer to administration of an active agent or composition of the invention to a subject for the purpose of effecting a therapeutic or prophylactic benefit through modulation of cathepsin S activity. Treating includes reversing, ameliorating, alleviating, inhibiting the progress of, lessening the severity of, or preventing a disease, disorder, or condition, or one or more symptoms of such disease, disorder or condition mediated through modulation of cathepsin S activity. The term “subject” refers to a mammalian patient in need of such treatment, such as a human. “Modulators” include both inhibitors and activators, where “inhibitors” refer to compounds that decrease, prevent, inactivate, desensitize or down-regulate cathepsin S expression or activity, and “activators” are compounds that increase, activate, facilitate, sensitize, or up-regulate cathepsin S expression or activity.

In treatment methods according to the invention, an effective amount of at least one active agent according to the invention is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.001 to about 200 mg of active agent per kg of subject's body weight per day, preferably about 0.05 to 100 mg/kg/day, or about 1 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the active agents of the invention may be used in combination with additional active ingredients in the treatment of the above conditions. The additional active ingredients may be coadministered separately with an active agent of Formula (I) or included with such an agent in a pharmaceutical composition according to the invention. In an exemplary embodiment, additional active ingredients are those that are known or discovered to be effective in the treatment of conditions, disorders, or diseases mediated by cathepsin S activity, such as another cathepsin S modulator or a compound active against another target associated with the particular condition, disorder, or disease. The combination may serve to increase efficacy (e.g., by including in the combination a compound potentiating the potency or effectiveness of an agent according to the invention), decrease one or more side effects, or decrease the required dose of the active agent according to the invention.

The active agents of the invention are used, alone or in combination with one or more additional active ingredients, to formulate pharmaceutical compositions of the invention. A pharmaceutical composition of the invention comprises: (a) an effective amount of at least one active agent in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active agents of the invention can be provided in the form of tablets or capsules, or as a solution, emulsion, or suspension. To prepare the oral compositions, the active agents may be formulated to yield a dosage of, e.g., from about 0.05 to about 50 mg/kg daily, or from about 0.05 to about 20 mg/kg daily, or from about 0.1 to about 10 mg/kg daily.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

Exemplary chemical entities useful in methods of the invention will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, artisans will note that the various transformations described in the following Schemes may be performed in a different order than that depicted. Unless otherwise specified, the variables are as defined above in reference to Formula (I).

Abbreviations and acronyms used herein include the following:

| Term | Acronym |
|---|---|
| Tetrahydrofuran | THF |
| N,N-Dimethylformamide | DMF |
| N,N-Dimethylacetamide | DMA |
| Dimethyl sulfoxide | DMSO |
| Ethyl acetate | EtOAc |
| tert-Butylcarbamoyl | Boc |
| Bovine serum albumin | BSA |
| High-pressure liquid chromatography | HPLC |
| Thin layer chromatography | TLC |
| Diisobutylaluminum hydride | DIBAL-H |
| Acetate | OAc |
| Acetic acid | AcOH |
| O-(7-Azabenzotriazol-1-yl)-N,N.N′,N′- | HATU |
| tetramethyluronium hexafluorophosphate | |
| Diisopropylethylamine | DIPEA |
| 4-(Dimethylamino)pyridine | DMAP |
| 1-(3-Dimethylaminopropyl)-3- | EDC |
| ethylcarbodiimide hydrochloride | |
| 1-Hydroxybenzotriazole | HOBt |
| Methanesulfonyl chloride | MsCl |
| (Trimethylsilyl)acetylene | TMSA |
| Triethylamine | TEA |
| 1,1′-Bis(diphenylphosphino)ferrocene | dppf |
| Trifluoroacetic acid | TFA |
| Diethyl ether | Et₂O |
| Methanol | MeOH |
| Ethanol | EtOH |
| Isopropanol | IPA |
| Benzotriazol-1-yl- | PyBOP |
| oxytripyrrolidinophosphonium | |
| hexafluorophosphate | |
| 1-Hydroxy-7-azabenzotriazole | HOAt |

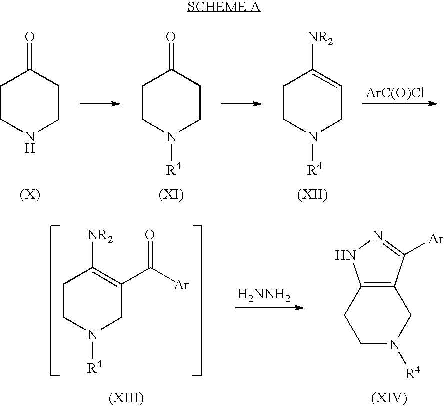

Referring to Scheme A, the tetrahydro-pyrazolo-pyridine core structure of Formula (I) may be prepared from commercially available piperidones (X). Installation of the R⁴ substituent is accomplished through, for example, alkylation, acylation, amide formation, or other suitable methods methods known in the art to provide ketones (XI). Alternatively, an amine protecting group, such as a Boc group, may be installed, and, at a later point in the synthesis, be removed (for example, by treatment with an acid such as HCl or TFA) and replaced with R⁴. Enamine formation according to general methods gives enamines (XII), which are then reacted with acyl chlorides, ArC(O)Cl, where Ar is a suitably substituted phenyl group, in the presence of a suitable tertiary amine base, to form enamines (XIII) or the corresponding beta-diketones, or a mixture thereof (not isolated). In situ reaction of the enamines with hydrazine generates pyrazoles (XIV).

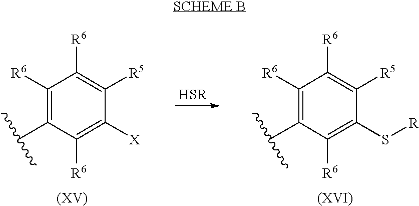

As shown in Scheme B, where Ar is a suitably substituted group as in Formula (XV), thioethers may be installed by any one of several methods to give compounds of Formula (XVI), including coupling with thiols HSR, where R is a suitable group that is or may be transformed into —CH₂C(R⁷)₂N(R⁸)R⁹ according to the subsequent Schemes or other standard methods. For example, thiols HSR where R is —CH₂CH₂N(R⁸)R⁹ may be prepared by reaction of amines HN(R⁸)R⁹ with thiirane. Where substituent X is iodide or bromide, coupling with thiols HSR is performed in the presence of a palladium catalyst, such as Pd₂ dba₃, optional additives such as dppf, a suitable base such as TEA, in a polar solvent such as DMF. Where substituent X is F, reaction with thiols HSR is done at elevated temperatures, preferably using microwave irradiation, in a polar solvent such as DMF. Alternatively, where R⁵ is an electron withdrawing group such as C₁ or CF₃ and substituent X is F or NO₂, displacement with thiols HSR may be accomplished in the presence of a suitable base. Any thioethers may be oxidized to the corresponding sulfoxides or sulfones (not shown) using methods known in the art, such as meta-chloroperbenzoic acid (mCPBA), dimethyldioxirane, or TeO₂/H₂O₂.

The chemistry described in Scheme B may be used with thiols HSR to generate thioether intermediates that are amenable to subsequent conversion into compounds of Formula (I). Examples are shown in Scheme C.

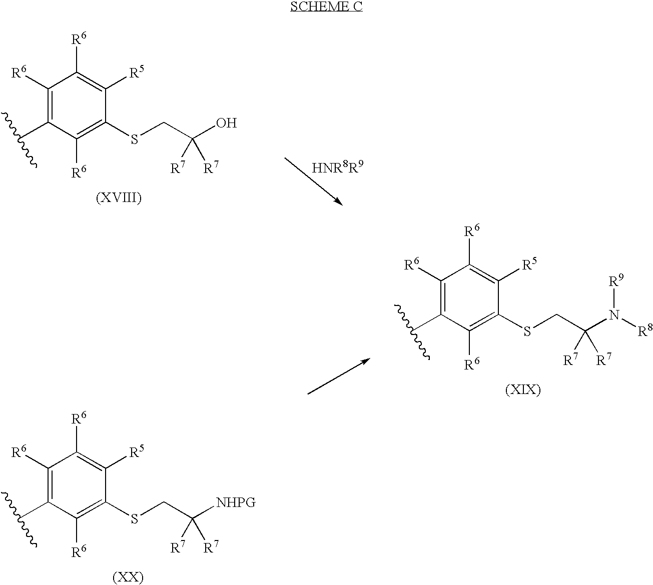

Thioethers (XVIII) may be converted using known methods into compounds of Formula (XIX), as depicted in Scheme C. Where the two R⁷ substituents are not carbonyl, activation of the hydroxy group and displacement with amines HNR⁸R⁹ give amines (XIX). Where the two R⁷ groups form a carbonyl group, acids (XVIII) may be coupled with amines HR⁸R⁹ using general amide coupling procedures. Also useful are aminoethyl derivatives (XX), where PG is a suitable nitrogen protecting group, such as a Boc group. Deprotection of the PG group and subsequent alkylation, amide formation, or reductive amination gives rise to amines or amides (XIX).

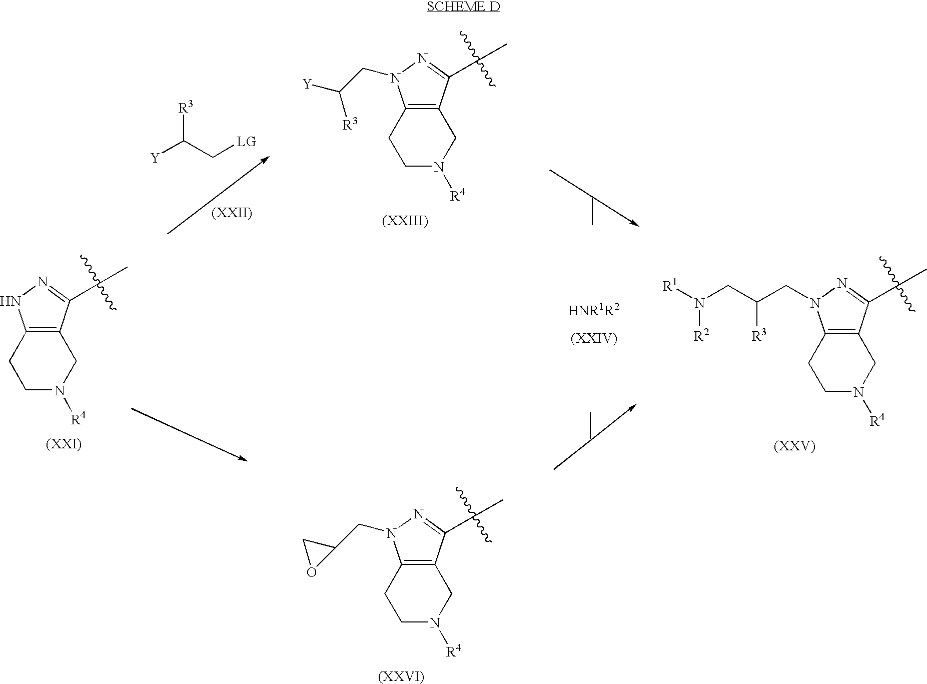

Two variations for the installation of the propyl amino chain are shown in Scheme D. One skilled in the art will recognize an amine protecting group, such as a Boc group, may be installed in place of the R⁴ shown, and, at a later point in the synthesis, be removed (for example, by treatment with an acid such as HCl or TFA) and replaced with R⁴. Pyrazoles (XXI) are alkylated with optionally protected reagents (XXII), where R³ is H, C₁₋₄alkyl, —OC₁₋₄alkyl, or a protected hydroxyl group, Y is an aldehyde, a protected aldehyde, —CH₂—OH, a —CH₂— (protected hydroxyl) group, —CH₂—Cl, or —CH₂NR¹R², and LG is a suitable leaving group, such as a chloride, bromide, iodide, mesylate or tosylate, to give compounds (XXIII). Where Y is a protected aldehyde (such as an acetal) or a —CH₂— (protected hydroxyl) group, deprotection of (XXIII) is accomplished under general conditions. Resulting aldehydes are reacted with amines (XXIV) under reductive amination conditions, to provide propyl amines (XXV) where R³ is H, C₁₋₄alkyl, or —OC₁₋₄alkyl. Alcohols are converted to suitable leaving groups (LG), and displaced with amines HNR¹R². Where Y is —CH₂NR¹R², the alkylation step leads to compounds (XXV) directly.

Alternatively, pyrazoles (XXI) are reacted with epichlorohydrin or glycidyinosylate, in the presence of a suitable base, to give epoxides (XXVI). Epoxide opening with amines (XXIV), preferably at elevated temperatures, yields propyl amines (XXV) where R³ is OH.

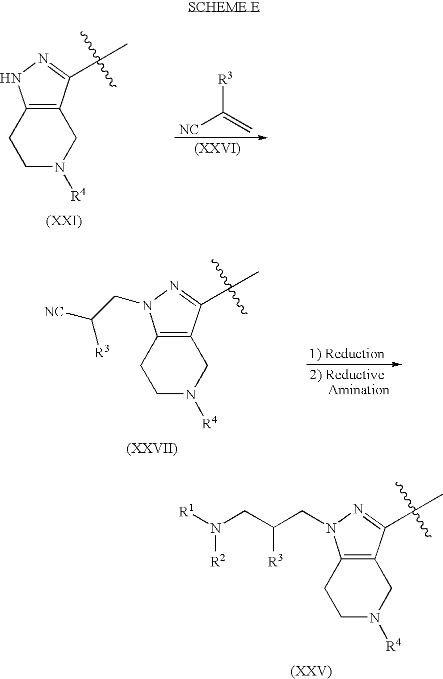

In another embodiment, shown in Scheme E, addition of pyrazoles (XXI) to α,β-unsaturated nitriles (XXVI), in the presence of a suitable base, such as aq. NaOH, generates nitriles (XXVII). Reduction of the nitriles to the corresponding aldehydes (XXIII, not shown) is accomplished with a reducing agent such as DIBAL-H. Reductive amination of aldehydes (XXIII) with amines (XXIV) gives amines (XXV) as described in Scheme D.

Compounds of Formula (I) may be converted to their corresponding salts using methods described in the art. For example, an amine of Formula (I) may be treated with trifluoroacetic acid, HCl, or citric acid in a solvent such as Et₂O, CH₂Cl₂, THF, or MeOH to provide the corresponding salt form.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following specific examples are provided to further illustrate the invention and various preferred embodiments.

### EXAMPLES

### Chemistry

In obtaining the compounds described in the examples below and the corresponding analytical data, the following experimental and analytical protocols were followed unless otherwise indicated.

Unless otherwise stated, reaction mixtures were magnetically stirred at room temperature (rt). Where solutions are “dried,” they are generally dried over a drying agent such as Na₂SO₄ or MgSO₄. Where mixtures, solutions, and extracts were “concentrated”, they were typically concentrated on a rotary evaporator under reduced pressure.

Microwave reactions were performed on a Personal Chemistry Emrys Optimizer. Individual reactions were heated to the desired temperature and held at that temperature for the allotted time.

Analytical HPLC retention times are reported in minutes, and were obtained on an Agilent HP-1100 instrument with a Phenomenex Luna C-18 (5 uM, 4.6×150 mm) column, with a flow rate of 1 mL/min, detection at 230, 254, and 280 nM, and a gradient of 10 to 100% CH₃CN (0.05% TFA)/H₂O (0.05% TFA). Alternatively, analytical reversed-phase HPLC was performed on an Agilent 1100 Series instrument using one of the following gradients: 1 to 99% acetonitrile/water (0.05% trifluoroacetic acid) over 5.0 min or 7.0 min with a flow rate of 1 mL/min (Waters XTerra MS C18 (5 μm, 4.6×100 mm) column or Phenomenex Synergi max-RP (4 μm, 4.6×150 mm) column) or 1 to 99% acetonitrile/water (20 mM NH₄OH) over 5.0 min or 7.0 min with a flow rate of 1.5 mL/min (Phenomenex Gemeni C18 (5 μm, 3.0×150 mm) column). Analytical reversed-phase LC/MS was performed either on an Agilent 1100 Series instrument using 5 to 99% acetonitrile/water (0.05% trifluoroacetic acid) over 5.0 min or 7.0 min with a flow rate of 0.6 mL/min (Waters XTerra RP18 (5 μm, 3.0×100 mm) column) or on a Waters 2790 instrument using 5 to 99% acetonitrile/water (0.1% formic acid) over 5.0 min with a flow rate of 0.6 mL/min (Waters XTerra RP18 (5 μm, 3.0×100 mm) column).

Preparatory HPLC purifications were typically performed on a Phenomenex Synergi column (4 μm, 21×150 mm), with a flow rate of 25 mL/min, and solvent conditions as described for Analytical HPLC. Alternatively, preparative reversed phase HPLC was performed on a Dionex APS2000 LC/MS or HPLC with a Phenomenex Gemini C18 (5 μm, 30×100 mm) column or a Waters XBridge C18 (5 μm, 30×100 mm) column and variable gradients of acetonitrile/water (20 mM NH₄OH) at a flow rate of 30 mL/min. Alternatively, the purification was performed with a Phenomenex Gemini C18 (5 μm, 50×100 mm) column or a Waters XBridge C18 (5 μm, 50×100 mm) column and variable gradients of acetonitrile/water (20 mM NH₄OH) at a flow rate of 80 mL/min.

Lyophilization of the mobile phase from preparatory HPLC runs afforded the free base, ammonium salt, or trifluoroacetate salt forms. Hydrochloric acid salts or citric acid salts were prepared by addition of 4 N HCl/1,4-dioxane, 2 M HCl/Et₂O, 1.25 M HCl/MeOH, or solid citric acid to solutions of the free base in CH₂Cl₂, followed by concentration.

Mass spectra (MS) were obtained on an Agilent series 1100 MSD using electrospray ionization (ESI) in positive mode unless otherwise indicated. Calculated (calcd.) mass corresponds to the exact mass. The MS data presented is the m/z found (typically [M+H]⁺) for the molecular ion.

Nuclear magnetic resonance (NMR) spectra were obtained on Bruker model DRX spectrometers (400, 500, or 600 MHz). The format of the ¹H NMR data below is: chemical shift in ppm downfield of the tetramethylsilane reference (multiplicity, coupling constant J in Hz, integration). All ¹H NMR data was acquired in CD₃OD solvent unless otherwise indicated. NMR interpretation was performed using MestReC software to assign chemical shift and multiplicity. In cases where 2 adjacent peaks of equal or unequal height were observed, these 2 peaks may be labeled either as a multiplet or as a doublet. In the case of a doublet a coupling constant using this software may be assigned.

Chemical names were generated using ChemDraw Version 6.0.2 (CambridgeSoft, Cambridge, Mass.) or ACD/Name Version 9 (Advanced Chemistry Development, Toronto, Ontario, Canada).

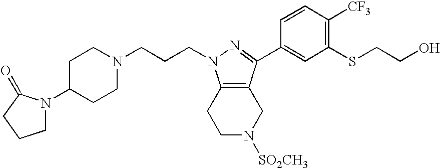

### Example 1

### 1-[1-(3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one

A. 1-Methanesulfonyl-piperidin-4-one. To a solution of 4-piperidone monohydrate hydrochloride (90 g, 586 mmol) in CHCl₃ (300 mL) and H₂O (300 mL) was added K₂CO₃ (324 g, 2340 mmol). The slurry was cooled to 0° C. and treated with methylsulfonyl chloride (MsCl; 136 mL, 1760 mmol) by drop-wise addition over a 1 h period (gas evolution was observed). The reaction mixture was allowed to shake for 72 h and was partitioned between CH₂Cl₂ (500 mL) and saturated (satd.) aq. NaHCO₃ (500 mL). The aqueous layer was extracted with CH₂Cl₂ (3×, 200 mL). The organic layer was washed with 1% KHSO₄ (250 mL), dried (Na₂SO₄), and concentrated to give 1-methanesulfonyl-piperidin-4-one (90.5 g, 87%) as a white solid. MS (ESI): mass calcd. for C₆H₁₁NO₃S, 177.05; m/z found, 178.1 [M+H]⁺. ¹H NMR (CDCl₃): 3.60 (t, J=6.5, 4H), 2.89 (s, 3H), 2.59 (t, J=6.3, 4H).

B. 5-Methanesulfonyl-3-(3-nitro-4-trifluoromethyl-phenyl)-4,5,6,7-tetrahydro-1H-pyrazolo[4,3-c]pyridine. p-Toluenesulfonic acid (0.12 g, 0.6 mmol) and morpholine (11.7 mL, 140.0 mmol) were added to a solution of 1-methanesulfonyl-piperidin-4-one (22.6 g, 127.6 mmol) in benzene (100 mL). The reaction mixture was heated at reflux in a flask equipped with a condenser and a Dean-Stark trap for 20 h. The reaction mixture was cooled and concentrated to give the enamine, which was used without further purification.

A 0° C. solution of the enamine (31.4 g, 127.6 mmol) in CH₂Cl₂ (60 mL) was treated with TEA (24.8 mL, 191.4 mmol) followed by drop-wise addition of a solution of 4-trifluoromethyl-3-nitrobenzoyl chloride* (32.3 g, 127.6 mmol) in CH₂Cl₂ (60 mL). The reaction mixture was allowed to warm to room temperature (rt) and stirred for 18 h. The reaction mixture was poured into H₂O (100 mL). The layers were separated, and the aqueous layer was extracted with CH₂Cl₂ (4×, 50 mL). The combined organic layers were dried (Na₂SO₄) and concentrated. The resulting oil was taken up in EtOH (200 mL) and treated with hydrazine (12.0 mL, 382.8 mmol) at 0° C. The reaction mixture was allowed to warm to rt and stirred for 18 h before concentration. The resulting material was taken up in CH₂Cl₂ and filtered. The crude solid was rinsed with additional CH₂Cl₂, 1 N HCl, and H₂O to afford a light yellow solid (18.9 g, 38%). ¹H NMR (DMSO-d₆): 8.31 (s, 1H), 8.12-8.04 (m, 2H), 4.54 (s, 2H), 3.56-3.50 (m, 2H), 3.00 (s, 3H), 2.89-2.84 (m, 2H). *Prepared by dissolving 4-trifluoromethyl-3-nitrobenzoic acid (30.0 g, 127.6 mmol) in toluene (350 mL) and thionyl chloride (11.2 mL, 153.1 mmol). A catalytic amount of DMF (850 μL) was added, and the mixture was stirred at rt for 15 h before heating to 50° C. for 6 h. After cooling to rt, the reaction mixture was concentrated and the resulting benzoyl chloride was used without further purification.

C. 2-[5-(5-Methanesulfonyl-4,5,6,7-tetrahydro-1H-pyrazolo[4,3-c]pyridin-3-yl)-2-trifluoromethyl-phenylsulfanyl]-ethanol. To a solution of 5-methanesulfonyl-3-(3-nitro-4-trifluoromethyl-phenyl)-4,5,6,7-tetrahydro-1H-pyrazolo[4,3-c]pyridine (4.0 g, 10.2 mmol) and K₂CO₃ (1.7 g, 12.3 mmol) in DMF (34 mL) was added 2-mercaptoethanol (2.9 mL, 40.9 mmol). The reaction mixture was heated to 70° C. for 13 h, cooled to rt, and poured into 1:1 brine/H₂O (100 mL). The resulting precipitate was filtered and rinsed with cold H₂O to provide an off-white solid (4.32 g, 100%). MS (ESI): mass calcd. for C₁₆H₁₈F₃N₃O₃S₂, 421.07; m/z found, 422.1 [M+H]⁺. ¹H NMR (DMSO-d₆): 7.81 (s, 1H), 7.77 (d, J=8.2, 1H), 7.52 (d, J=8.2, 1H), 5.10 (br s, 1H), 4.49 (s, 2H), 3.67 (t, J=6.6, 2H), 3.52 (t, J=5.8, 2H), 3.22 (t, J=6.6, 2H), 3.00 (s, 3H), 2.85 (t, J=5.7, 2H).

D. 2-[5-[1-(2-[1,3]Dioxolan-2-yl-ethyl)-5-methanesulfonyl-4,5,6,7-tetrahydro-1H-pyrazolo[4,3-p]pyridin-3-yl]-2-trifluoromethyl-phenylsulfanyl]-ethanol. To a solution of 2-[5-(5-methanesulfonyl-4,5,6,7-tetrahydro-1H-pyrazolo[4,3-c]pyridin-3-yl)-2-trifluoromethyl-phenylsulfanyl]-ethanol (3.0 g, 7.1 mmol) and Cs₂CO₃ (4.6 g, 14.2 mmol) in DMF (30 mL) was added 2-(2-bromoethyl)-1,3-dioxolane (1.3 mL, 10.7 mmol). The reaction mixture was stirred at rt for 18 h and poured into cold satd. aq. NH₄Cl (100 mL). The resulting precipitate was filtered and rinsed with H₂O to afford an off-white solid (3.3 g, 93%) that was used without further purification. MS (ESI): mass calcd. for C₂₁H₂₆F₃N₃O₅S₂, 521.13; m/z found, 522.1 [M+H]⁺. ¹H NMR (DMSO-d₆): 7.76 (s, 1H), 7.55 (d, J=8.5, 1H), 7.08 (br s, 1H), 5.05 (t, J=5.4, 1H), 4.84 (t, J=4.6, 1H), 4.47 (s, 2H), 4.19-4.12 (m, 2H), 3.93-3.90 (m, 2H), 3.80-3.77 (m, 2H), 3.67-3.62 (m, 2H), 3.55-3.50 (m, 2H), 3.18 (t, J=6.6, 2H), 2.99 (s, 3H), 2.89-2.86 (m, 2H).

E. 3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl-propionaldehyde. To a solution of 2-{5-[1-(2-[1,3]dioxolan-2-yl-ethyl)-5-methanesulfonyl-4,5,6,7-tetrahydro-1H-pyrazolo[4,3-c]pyridin-3-yl]-2-trifluoromethyl-phenylsulfanyl}-ethanol (2.0 g, 3.8 mmol) in acetone (32 mL) was added 1 N HCl (9.0 mL, 9.0 mmol). The reaction mixture was heated to 55° C. for 6 h or until reaction completion as monitored by HPLC. Additional aliquots of 1 N HCl were added if reaction conversion was slow. After concentration, the resulting residue was re-dissolved in 1:1 CH₂Cl₂/1 N HCl (60 mL) and the layers were separated. The aqueous layer was extracted with CH₂Cl₂ (3×, 30 mL), and the combined organic layers were dried (Na₂SO₄) and concentrated to afford a yellow foam that was used without further purification. MS (ESI): mass calcd. for C₁₉H₂₂F₃N₃O₄S₂, 477.10; m/z found, 478.3 [M+H]⁺.

F. 1-[1-(3-{3-[3-(2-Hydroxy-ethylsulfanyl)-4-trifluoromethyl-phenyl]-5-methanesulfonyl-4,5,6,7-tetrahydro-pyrazolo[4,3-p]pyridin-1-yl}-propyl)-piperidin-4-yl]-pyrrolidin-2-one. To a solution of 3-{3-[3-(2-hydroxy-ethylsulfanyl)-4-trifluoromethyl-phenyl]-5-methanesulfonyl-4,5,6,7-tetrahydro-pyrazolo[4,3-c]pyridin-1-yl}propionaldehyde (1.8 g, 3.8 mmol) in CH₂Cl₂ (20 mL) was added 4-(N-2-pyrrolidinone)piperidine (1.0 g, 5.7 mmol) and glacial acetic acid (0.8 mL, 14 mmol). After stirring for 5 min, NaB(OAc)₃H (1.2 g, 5.7 mmol) was added to the reaction mixture. After stirring at rt for 15 h, 1 N NaOH (20 mL) was added to the mixture, and the layers were separated. The aqueous layer was extracted with CH₂Cl₂ (4×, 20 mL), and the combined organic layers were dried (Na₂SO₄) and concentrated to afford a yellow oil. Purification (SiO₂; 0-10% 1:9 satd. NH₃ in MeOH/MeOH in CH₂Cl₂) provided a yellow foam (1.3 g, 53%), which could be used without further purification. Or, reverse phase HPLC of this yellow foam gave a white powder. MS (ESI): mass calcd. for C₂₈H₃₈F₃N₅O₄S₂, 629.23; m/z found, 630.2 [M+H]⁺. ¹H NMR: 7.97 (s, 1H), 7.77 (d, J=8.2, 1H), 7.59 (d, J=8.2, 1H), 4.57 (s, 2H), 4.31-4.28 (m, 2H), 4.14-4.07 (m, 1H), 3.81 (t, J=6.7, 2H), 3.73-3.64 (m, 4H), 3.32-3.22 (m, 6H), 3.17-3.08 (m, 2H), 3.01 (s, 3H), 2.99-2.92 (m, 2H), 2.43-2.32 (m, 4H), 2.10-2.00 (m, 3H), 2.00-1.92 (m, 3H).

The compounds in Examples 2 and 3 were prepared using methods analogous to those described in Example 1.

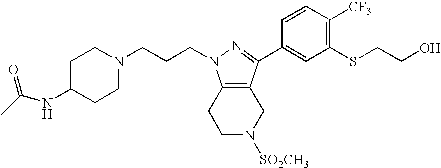

### Example 2

### N-[1-(3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-acetamide

MS (ESI): mass calcd. for C₂₆H₃₆F₃N₅O₄S₂, 603.22; m/z found, 604.5 [M+H]⁺.

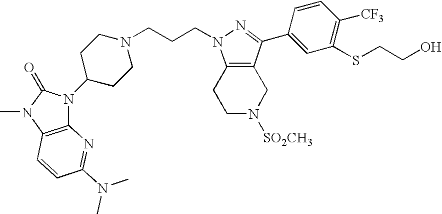

### Example 3

### 5-Dimethylamino-3-[1-(3-{3-[3-(2-hydroxy-ethylsulfanyl)-4-trifluoromethyl-phenyl]-5-methanesulfonyl-4,5,6,7-tetrahydro-pyrazolo[4,3-c]pyridin-1-yl}-propyl) piperidin-4-yl]-1-methyl-1,3-dihydro-imidazo[4,5-b]pyridin-2-one

MS (ESI): mass calcd. for C₃₃H₄₃F₃N₈O₄S₂, 736.28; m/z found, 737.4 [M+H]⁺.

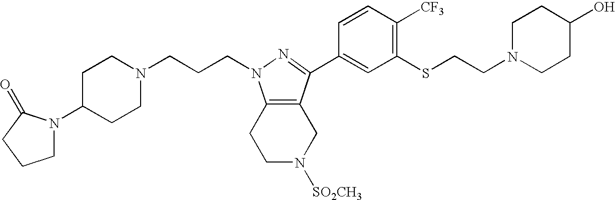

### Example 4

### 1-{1-[3-(3-{3-[2-(4-Hydroxy-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

A. Methanesulfonic acid 2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl ester. To a solution of 1-[1-(3-{3-[3-(2-hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one (0.2 g, 0.3 mmol) in CH₂Cl₂ (1.5 mL) was added pyridine (0.05 mL, 0.6 mmol). MsCl (0.04 mL, 0.5 mmol) was slowly added, and the reaction mixture was stirred for 1 h. After pouring the mixture into H₂O (5 mL), the layers were separated, and the aqueous layer was extracted with CH₂Cl₂ (3×, 5 mL). The combined organic layers were washed with brine (5 mL), dried (Na₂SO₄), and concentrated to afford a yellow-brown foam, which was used without further purification. MS (ESI): mass calcd. for C₂₉H₄₀F₃N₅O₆S₃, 707.21; m/z found, 708.2 [M+H]⁺.

B. 1-{1-[3-(3-{3-[2-(4-Hydroxy-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrolidin-2-one. To a solution of methanesulfonic acid 2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl ester (0.2 g, 0.3 mmol) in 1:3 dichloroethane/EtOH (2 mL) was added 4-hydroxypiperidine (0.6 g, 6 mmol). The reaction mixture was heated to 60° C. for 15 h and concentrated to provide an orange oil. Further purification using reverse phase HPLC led to a yellow solid (148 mg, 56%). MS (ESI): mass calcd. for C₃₃H₄₇F₃N₆O₄S₂, 712.31; m/z found, 713.5 [M+H]⁺. ¹H NMR: 7.92-7.90 (m, 1H), 7.78 (d, J=8.2, 1H), 7.68-7.65 (m, 1H), 4.53 (s, 2H), 4.23 (t, J=6.5, 2H), 4.10-4.03 (m, 1H), 3.66-3.58 (m, 5H), 3.48-3.38 (m, 3H), 3.38-3.28 (m, 5H), 3.22-3.18 (m, 2H), 3.10-3.02 (m, 3H), 2.95 (s, 3H), 2.93-2.90 (m, 2H), 2.35-2.30 (m, 4H), 2.11-2.05 (m, 1H), 2.02-1.92 (m, 6H), 1.91-1.84 (m, 3H), 1.72-1.63 (m, 1H).

Examples 5-53 were prepared according to the methods described in Example 4, with the appropriate substituent changes. In the cases where hydrogen chloride salts of the amine reagent were used, TEA (20 equiv) was added to the reaction mixture.

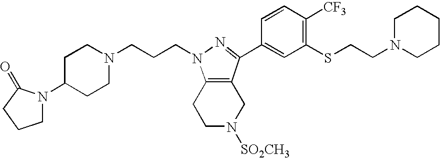

### Example 5

### 1-[1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one

MS (ESI): mass calcd. for C₃₃H₄₇F₃N₆O₃S₂, 696.31; m/z found, 697.5 [M+H]⁺. ¹H NMR: 7.94 (s, 1H), 7.83 (d, J=8.2, 1H), 7.73 (d, J=8.2, 1H), 4.56 (s, 2H), 4.26 (t, J=6.5, 2H), 4.15-4.06 (m, 1H), 3.68-3.65 (m, 2H), 3.66-3.64 (m, 2H), 3.59-3.56 (m, 2H), 3.59-3.46 (m, 2H), 3.36-3.32 (m, 4H), 3.31-3.30 (m, 3H), 3.24-3.21 (m, 2H), 3.12-3.08 (m, 2H), 2.96-2.94 (m, 4H), 2.38-2.33 (m, 4H), 2.05-1.98 (m, 4H), 1.96-1.90 (m, 4H), 1.85-1.70 (m, 3H), 1.55-1.43 (m, 1H).

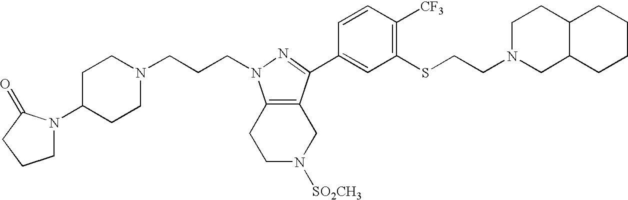

### Example 6

### 1-{1-[3-(5-Methanesulfonyl-3-{3-[2-(octahydro-isoquinolin-2-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₇H₅₃F₃N₆O₃S₂, 750.36; m/z found, 751.5 [M+H]⁺. ¹H NMR: 7.85 (s, 1H), 7.75-7.73 (m, 1H), 7.65-7.63 (m, 1H), 4.47 (s, 2H), 4.17 (t, J=6.5, 2H), 4.04-3.96 (m, 1H), 3.60-3.54 (m, 4H), 3.47-3.44 (m, 1H), 3.40-3.36 (m, 2H), 3.32-3.28 (m, 3H), 3.26-3.21 (m, 1H), 3.18-3.10 (m, 3H), 3.04-2.94 (m, 3H), 2.89 (s, 3H), 2.87-2.85 (m, 2H), 2.30-2.24 (m, 4H), 1.98-1.82 (m, 8H), 1.80-1.75 (m, 1H), 1.72-1.66 (m, 1H), 1.65-1.60 (m, 1H), 1.58-1.50 (m, 3H), 1.44-1.35 (m, 2H), 1.30-1.14 (m, 3H).

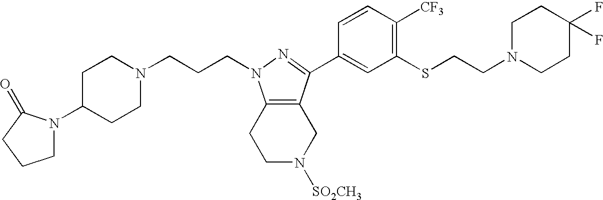

### Example 7

### 1-{1-[3-(3-{3-[2-(4,4-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₅F₅N₆O₃S₂, 732.29; m/z found, 733.4 [M+H]⁺. ¹H NMR: 7.84 (s, 1H), 7.75-7.73 (m, 1H), 7.66-7.64 (m, 1H), 4.47 (s, 2H), 4.17 (t, J=6.5, 2H), 4.03-3.95 (m, 1H), 3.59-3.56 (m, 3H), 3.55-3.53 (m, 2H), 3.42-3.33 (m, 8H), 3.25-3.20 (m, 1H), 3.17-3.12 (m, 2H), 3.05-2.97 (m, 2H), 2.90 (s, 3H), 2.86-2.82 (m, 2H), 2.30-2.30 (m, 8H), 1.96-1.80 (m, 6H).

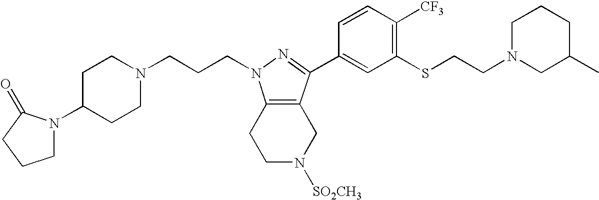

### Example 8

### 1-{1-[3-(5-Methanesulfonyl-3-{3-[2-(3-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₉F₃N₆O₃S₂, 710.33; m/z found, 711.5 [M+H]⁺. ¹H NMR: 7.85 (s, 1H), 7.73 (m, 1H), 7.63 (m, 1H), 4.47 (s, 2H), 4.17 (t, J=6.5, 2H), 4.04-3.97 (m, 1H), 3.60-3.55 (m, 4H), 3.51-3.46 (m, 1H), 3.41-3.37 (m, 3H), 3.29-3.22 (m, 4H), 3.15-3.12 (m, 2H), 3.02-1.98 (m, 2H) 2.89 (s, 3H), 2.87-2.84 (m, 2H), 2.79-2.74 (m, 1H), 2.50-2.48 (m, 1H), 2.30-2.22 (m, 4H), 1.96-1.62 (m, 10H), 1.12-1.02 (m, 1H), 0.90-0.89 (m, 3H).

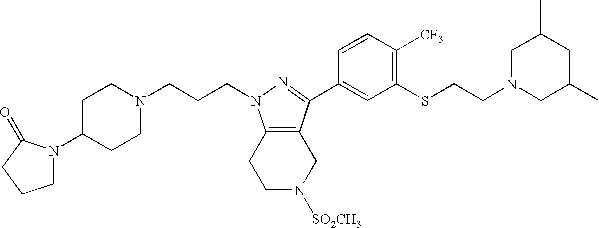

### Example 9

### 1-{1-[3-(3-{3-[2-(3,5-Dimethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₅H₅₁F₃N₆O₃S₂, 724.34; m/z found, 725.5 [M+H]⁺. ¹H NMR: 7.85 (s, 1H), 7.73 (d, J=8.1, 1H), 7.62 (d, J=8.1, 1H), 4.47 (s, 2H), 4.18-4.15 (m, 2H), 4.04-3.98 (m, 1H), 3.58-3.55 (m, 4H), 3.42-3.36 (m, 4H), 3.29-3.22 (m, 4H), 3.15-3.10 (m, 2H), 3.02-2.98 (m, 2H), 2.89 (s, 3H), 2.87-2.84 (m, 2H), 2.45-2.51 (m, 2H), 2.28-2.22 (m, 4H), 1.95-1.90 (m, 4H), 1.85-1.77 (m, 4H), 1.75-1.73 (m, 1H), 0.88 (d, J=6.5, 6H), 0.80-0.73 (m, 1H).

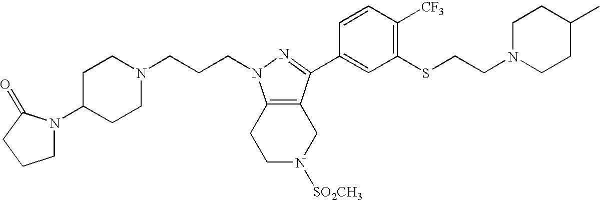

### Example 10

### 1-{1-[3-(5-Methanesulfonyl-3-{3-[2-(4-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₉F₃N₆O₃S₂, 710.33; m/z found, 711.5 [M+H]⁺. ¹H NMR: 7.84 (s, 1H), 7.74 (d, J=8.2, 1H), 7.64 (d, J=8.2, 1H), 4.47 (s, 2H), 4.17 (t, J=6.5, 2H), 4.04-3.97 (m, 1H), 3.59-3.54 (m, 4H), 3.51-3.47 (m, 2H), 3.39-3.36 (m, 2H), 3.28-3.22 (m, 4H), 3.16-3.11 (m, 2H), 3.04-2.97 (m, 2H), 2.89 (s, 3H), 2.87-2.84 (m, 4H), 2.30-2.22 (m, 4H), 1.95-1.87 (m, 4H), 1.85-1.80 (m, 4H), 1.62-1.56 (m, 1H), 1.35-1.28 (m, 2H), 0.92-0.89 (s, 3H).

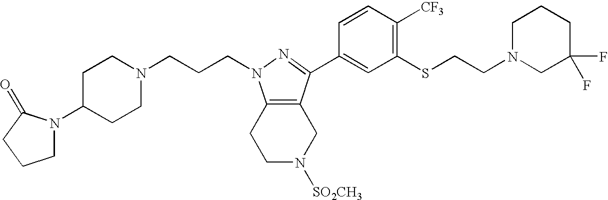

### Example 11

### 1-{1-[3-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₅F₅N₆O₃S₂, 732.29; m/z found, 733.4 [M+H]⁺. ¹H NMR: 7.85 (s, 1H), 7.73 (d, J=8.2, 1H), 7.60 (d, J=8.2, 1H), 4.47 (s, 2H), 4.17 (t, J=6.5, 2H), 4.02-3.96 (m, 1H), 3.58-3.52 (m, 5H), 3.36-3.31 (m, 5H), 3.24-3.21 (m, 2H), 3.17-3.12 (m, 2H), 3.08-2.98 (m, 4H), 2.89 (s, 3H), 2.87-2.85 (m, 2H), 2.29-2.22 (m, 4H), 2.03-1.80 (m, 10H).

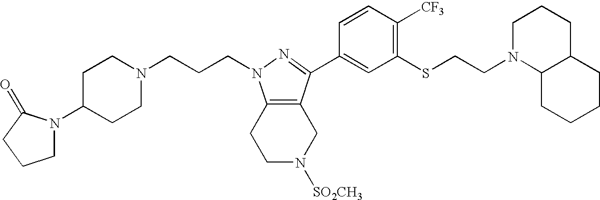

### Example 12

### 1-{1-[3-(5-Methanesulfonyl-3-{3-[2-(octahydro-quinolin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₇H₅₃F₃N₆O₃S₂, 750.36; m/z found, 751.5 [M+H]⁺. ¹H NMR: 7.89 (s, 1H), 7.75 (d, J=8.2, 1H), 7.65 (d, J=8.2, 1H), 4.47 (s, 2H), 4.17 (t, J=6.5, 2H), 4.03-3.97 (m, 1H), 3.59-3.49 (m, 4H), 3.50-3.45 (m, 1H), 3.42-3.37 (m, 2H), 3.34-3.30 (m, 1H), 3.27-3.22 (m, 4H), 3.15-3.11 (m, 2H), 3.08-2.98 (m, 3H), 2.90 (s, 3H), 2.87-2.85 (m, 2H), 2.84-2.81 (m, 1H), 2.29-2.22 (m, 4H), 1.96-1.82 (m, 7H), 1.73-1.63 (m, 4H), 1.59-1.57 (m, 1H), 1.44-1.38 (m, 1H), 1.25-1.14 (m, 4H), 1.08-1.02 (m, 1H).

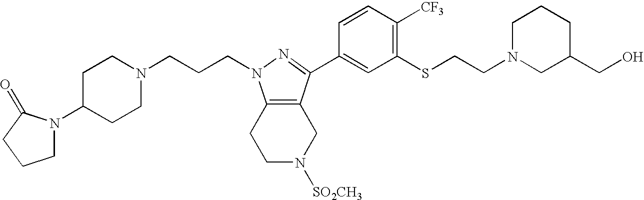

### Example 13

### 1-{1-[3-(3-{3-[2-(3-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₉F₃N₆O₄S₂, 726.32; m/z found, 727.5 [M+H]⁺. ¹H NMR: 7.86 (s, 1H), 7.74 (d, J=8.2, 1H), 7.64-7.62 (m, 1H), 4.47 (s, 2H), 4.17 (t, J=6.5, 2H), 4.03-3.97 (m, 1H), 3.60-3.49 (m, 6H), 3.46-3.42 (m, 1H), 3.40-3.37 (m, 2H), 3.34-3.28 (m, 3H), 3.26-3.21 (m, 3H), 3.18-3.15 (m, 2H), 3.03-2.98 (m, 2H), 2.89 (s, 3H), 2.87-2.85 (m, 2H), 2.83-2.77 (m, 1H), 2.68-2.63 (m, 1H), 2.29-2.22 (m, 4H), 1.96-1.80 (m, 8H), 1.75-1.64 (m, 2H), 1.21-1.14 (m, 1H).

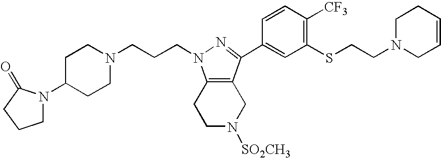

### Example 14

### 1-{1-[3-(3-{3-[2-(3,6-Dihydro-2H-pyridin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₅F₃N₆O₃S₂, 694.29; m/z found, 695.4 [M+H]⁺. ¹H NMR: 7.91 (s, 1H), 7.79 (d, J=8.2, 1H), 7.70-7.68 (m, 1H), 5.96-5.91 (m, 1H), 5.72-5.68 (m, 1H), 4.52 (s, 2H), 4.22 (t, J=6.5, 2H), 4.09-4.02 (m, 1H), 3.94-3.83 (m, 1H), 3.64-3.59 (m, 5H), 3.46-3.40 (m, 5H), 3.32-3.27 (m, 3H), 3.20-3.16 (m, 2H), 3.08-3.02 (m, 2H), 2.94 (s, 3H), 2.93-2.89 (m, 2H), 2.51-2.35 (m, 2H), 2.34-2.27 (m, 4H), 2.00-1.95 (m, 4H), 1.90-1.85 (m, 2H).

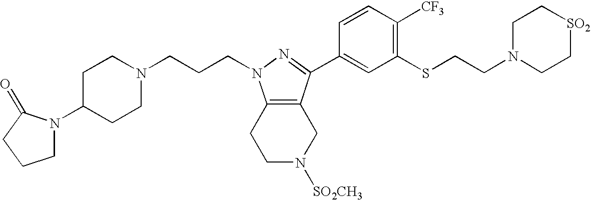

### Example 15

### 1-{1-[3-(3-{3-[2-(1,1-Dioxo-1λ⁶-thiomorpholin-4-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₂H₄₅F₃N₆O₅S₃, 746.26; m/z found, 747.4 [M+H]⁺. ¹H NMR: 7.87 (s, 1H), 7.68 (d, J=8.2, 1H), 7.52 (d, J=8.2, 1H), 4.45 (s, 2H), 4.17 (t, J=6.5, 2H), 4.02-3.96 (m, 1H), 3.59-3.53 (m, 4H), 3.21-3.18 (m, 2H), 3.18-3.13 (m, 2H), 3.09-3.05 (m, 5H), 3.04-3.00 (m, 5H), 2.89 (s, 3H), 2.88-2.84 (m, 4H), 2.30-2.21 (m, 5H), 1.95-1.89 (m, 3H), 1.88-1.82 (m, 4H).

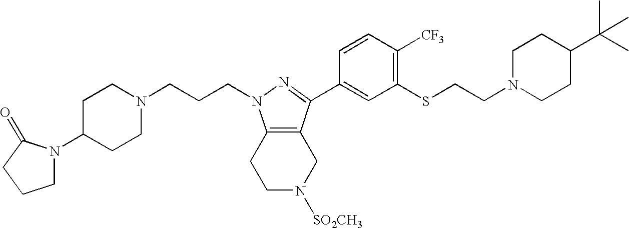

### Example 16

### 1-{1-[3-(3-{3-[2-(4-tert-Butyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₇H₅₅F₃N₆O₃S₂, 752.37; m/z found, 753.5 [M+H]⁺. ¹H NMR: 7.84 (s, 1H), 7.74 (d, J=8.2, 1H), 7.65 (d, J=8.2, 1H), 4.47 (s, 2H), 4.17 (t, J=6.5, 2H), 4.02-3.98 (m, 1H), 3.60-3.52 (m, 6H), 3.39-3.35 (m, 2H), 3.27-3.21 (m, 3H), 3.15-3.12 (m, 2H), 3.04-2.97 (m, 2H), 2.89 (s, 3H), 2.87-2.83 (m, 3H), 2.30-2.22 (m, 4H), 1.98-1.80 (m, 10H), 1.47-1.39 (m, 2H), 1.30-1.24 (m, 1H), 0.81 (s, 9H).

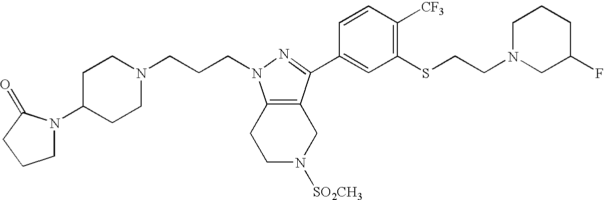

### Example 17

### 1-{1-[3-(3-{3-[2-(3-Fluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₆F₄N₆O₃S₂, 714.30; m/z found, 715.5 [M+H]⁺. ¹H NMR: 7.85 (s, 1H), 7.75 (d, J=8.2, 1H), 7.67-7.65 (m, 1H), 4.47 (s, 2H), 4.17 (t, J=6.5, 2H), 4.04-3.96 (m, 1H), 3.77-3.65 (m, 1H), 3.59-3.53 (m, 5H), 3.46-3.45 (m, 1H), 3.41-3.36 (m, 2H), 3.34-3.30 (m, 2H), 3.27-3.23 (m, 3H), 3.16-3.12 (m, 2H), 3.04-2.97 (m, 3H), 2.90 (s, 3H), 2.87-2.85 (m, 2H), 2.30-2.22 (m, 4H), 2.04-1.76 (m, 10H).

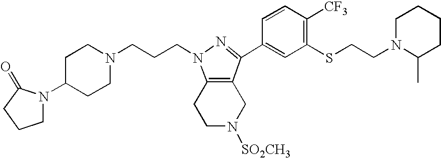

### Example 18

### 1-{1-[3-(5-Methanesulfonyl-3-{3-[2-(2-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₉F₃N₆O₃S₂, 710.33; m/z found, 711.5 [M+H]⁺. ¹H NMR: 7.88 (s, 1H), 7.75 (d, J=8.2, 1H), 7.66-7.63 (m, 1H), 4.47 (s, 2H), 4.17 (t, J=6.5, 2H), 4.04-3.97 (m, 1H), 3.60-3.55 (m, 4H), 3.50-3.40 (m, 3H), 3.37-3.31 (m, 2H), 3.26-3.20 (m, 4H), 3.14-3.11 (m, 2H), 3.04-2.97 (m, 3H), 2.89 (s, 3H), 2.87-2.85 (m, 2H), 2.29-2.22 (m, 4H), 1.95-1.80 (m, 8H), 1.78-1.70 (m, 2H), 1.34-1.32 (m, 1H), 1.20 (d, J=6.5, 3H).

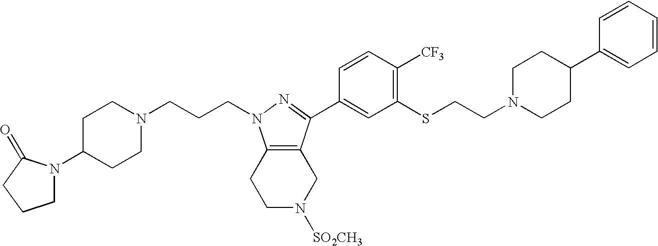

### Example 19

### 1-{1-[3-(5-Methanesulfonyl-3-{3-[2-(4-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₉H₅₁F₃N₆O₃S₂, 772.34; m/z found, 773.5 [M+H]⁺. ¹H NMR: 7.87 (s, 1H), 7.76 (d, J=8.2, 1H), 7.68-7.66 (m, 1H), 7.24-7.21 (m, 2H), 7.15-7.12 (m, 3H), 4.49 (s, 2H), 4.19-4.16 (m, 2H), 4.02-3.96 (m, 1H), 3.66-3.60 (m, 2H), 3.60-3.54 (m, 5H), 3.47-3.45 (m, 1H), 3.44-3.40 (m, 2H), 3.37-3.32 (m, 3H), 3.16-3.12 (m, 2H), 3.10-3.05 (m, 2H), 3.04-2.96 (m, 2H), 2.90 (s, 3H), 2.88-2.85 (m, 2H), 2.81-2.76 (m, 1H), 2.29-2.22 (m, 4H), 2.05-2.00 (m, 2H), 1.98-1.82 (m, 7H).

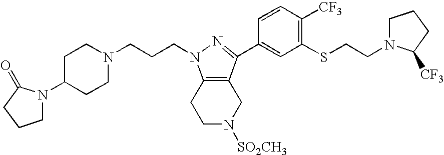

### Example 20

### (S)-1-{1-[3-(5-Methanesulfonyl-3-{4-trifluoromethyl-3-[2-(2-trifluoromethyl-pyrrolidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₄F₆N₆O₃S₂, 750.28; m/z found, 751.4 [M+H]⁺. ¹H NMR: 7.79 (s, 1H), 7.67 (d, J=8.2, 1H), 7.50 (d, J=8.2, 1H), 4.44 (s, 2H), 4.17 (t, J=6.4, 2H), 4.02-3.96 (m, 1H), 3.62-3.58 (m, 1H), 3.57-3.50 (m, 3H), 3.30-3.25 (m, 2H), 3.22-3.12 (m, 5H), 3.13-3.06 (m, 1H), 3.03-2.96 (m, 2H), 2.87 (s, 3H), 2.86-2.84 (m, 3H), 2.50-2.45 (m, 1H), 2.28-2.21 (m, 4H), 2.00-1.88 (m, 3H), 1.88-1.70 (m, 7H).

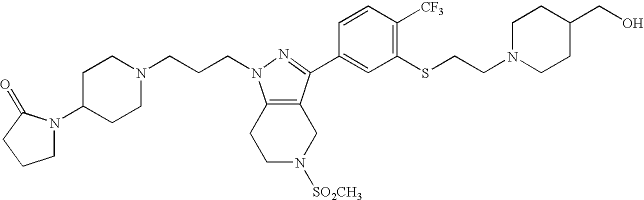

### Example 21

### 1-{1-[3-(3-{3-[2-(4-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₉F₃N₆O₄S₂, 726.32; m/z found, 727.5 [M+H]⁺. ¹H NMR: 7.91 (s, 1H), 7.80 (d, J=8.2, 1H), 7.69 (d, J=8.2, 1H), 4.54 (s, 2H), 4.23 (t, J=6.5, 2H), 4.11-4.04 (m, 1H), 3.67-3.60 (m, 6H), 3.48-3.44 (m, 2H), 3.42-3.38 (m, 2H), 3.36-3.28 (m, 4H), 3.23-3.17 (m, 2H), 3.10-3.04 (m, 2H), 3.02-2.95 (m, 2H), 2.96 (s, 3H), 2.94-2.90 (m, 2H), 2.36-2.30 (m, 4H), 2.02-1.94 (m, 6H), 1.92-1.86 (m, 2H), 1.75-1.68 (m, 1H), 1.50-1.42 (m, 2H).

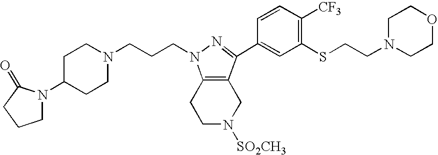

### Example 22

### 1-[1-(3-{5-Methanesulfonyl-3-[3-(2-morpholin-4-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one

MS (ESI): mass calcd. for C₃₂H₄₅F₃N₆O₄S₂, 698.29; m/z found, 699.5 [M+H]⁺. ¹H NMR: 7.89 (s, 1H), 7.78 (d, J=8.2, 1H), 7.68 (d, J=8.2, 1H), 4.52 (s, 2H), 4.21 (t, J=6.5, 2H), 4.08-4.02 (m, 1H), 4.05-3.92 (m, 2H), 3.79-3.67 (m, 2H), 3.63-3.60 (m, 4H), 3.54-3.43 (m, 4H), 3.40-3.35 (m, 3H), 3.31-3.28 (m, 2H), 3.20-3.14 (m, 3H), 3.09-3.01 (m, 2H), 2.94 (s, 3H), 2.91-2.89 (m, 2H), 2.35-2.27 (m, 4H), 2.00-1.93 (m, 4H), 1.90-1.85 (m, 2H).

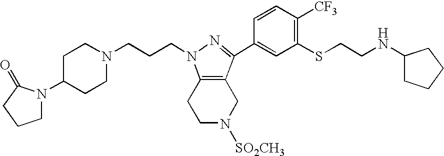

### Example 23

### 1-[1-(3-{3-[3-(2-Cyclopentylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₃H₄₇F₃N₆O₃S₂, 696.31; m/z found, 697.5 [M+H]⁺. ¹H NMR: 7.87 (s, 1H), 7.73 (d, J=8.2, 1H), 7.59 (d, J=8.2, 1H), 4.45 (s, 2H), 4.16 (t, J=6.5, 2H), 2.05-1.98 (m, 1H), 3.59-3.54 (m, 4H), 3.52-3.45 (m, 1H), 3.30-3.22 (m, 4H), 3.19-3.15 (m, 2H), 3.15-3.11 (m, 2H), 3.15-2.97 (m, 2H), 2.89 (s, 3H), 2.86-2.84 (m, 2H), 2.30-2.24 (m, 4H), 2.02-1.88 (m, 6H), 1.84-1.80 (m, 2H), 1.71-1.68 (m, 2H), 1.58-1.48 (m, 5H).

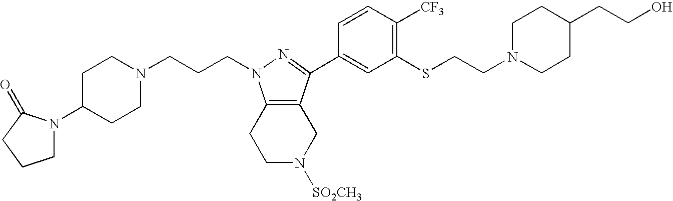

### Example 24

### 1-(1-{3-[3-(3-{2-[4-(2-Hydroxy-ethyl)-piperidin-1-yl]-ethylsulfanyl}-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one

MS (ESI): mass calcd. for C₃₅H₅₁F₃N₆O₄S₂, 740.34; m/z found, 741.5 [M+H]⁺. ¹H NMR: 7.91 (s, 1H), 7.79 (d, J=8.2, 1H), 7.68 (d, J=8.2, 1H), 4.53 (s, 2H), 4.23 (t, J=6.5, 2H), 4.11-4.05 (m, 1H), 3.66-3.61 (m, 4H), 3.61-3.56 (m, 4H), 3.47-3.43 (m, 2H), 3.35-3.29 (m, 4H), 3.22-3.17 (m, 2H), 3.10-3.03 (m, 2H), 2.99-2.96 (m, 2H), 2.96 (s, 3H), 2.94-2.90 (m, 2H), 2.35-2.29 (m, 4H), 2.02-1.94 (m, 6H), 1.91-1.86 (m, 2H), 1.76-1.68 (m, 1H), 1.49-1.38 (m, 4H).

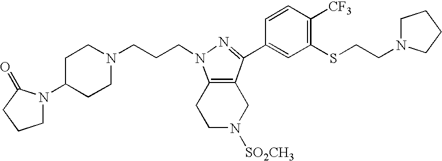

### Example 25

### 1-[1-(3-{5-Methanesulfonyl-3-[3-(2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one

MS (ESI): mass calcd. for C₃₂H₄₅F₃N₆O₃S₂, 682.29; m/z found, 683.5 [M+H]⁺. ¹H NMR: 7.85 (s, 1H), 7.73 (d, J=8.2, 1H), 7.62 (d, J=8.2, 1H), 4.46 (s, 2H), 4.16 (t, J=6.5, 2H), 4.04-3.97 (m, 1H), 3.65-3.58 (m, 2H), 3.58-3.53 (m, 4H), 3.39-3.33 (m, 4H), 3.25-3.23 (m, 2H), 3.14-3.11 (m, 2H), 3.02-2.98 (m, 4H), 2.89 (s, 3H), 2.86-2.84 (m, 2H), 2.29-2.21 (m, 4H), 2.08-1.98 (m, 2H), 1.95-1.86 (m, 6H), 1.85-1.79 (m, 2H).

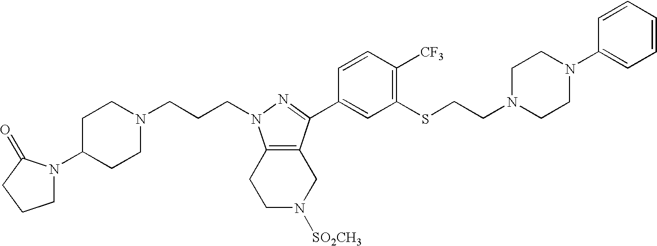

### Example 26

### 1-{1-[3-(5-Methanesulfonyl-3-{3-[2-(4-phenyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₈H₅₀F₃N₇O₃S₂, 773.34; m/z found, 774.5 [M+H]⁺. ¹H NMR: 7.97 (s, 1H), 7.85 (d, J=8.2, 1H), 7.75-7.74 (m, 1H), 7.30-7.26 (m, 2H), 7.01-6.99 (m, 2H), 6.94-6.91 (m, 1H), 4.58 (s, 2H), 4.27 (t, J=6.6, 2H), 4.11-4.05 (m, 1H), 3.70-3.62 (m, 6H), 3.57-3.47 (m, 7H), 3.35-3.32 (m, 3H), 3.25-3.20 (m, 3H), 3.12-3.05 (m, 2H), 2.99 (s, 3H), 2.98-2.94 (m, 2H), 2.38-2.31 (m, 4H), 2.05-1.87 (m, 7H).

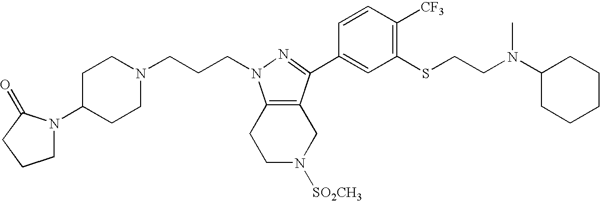

### Example 27

### 1-{1-[3-(3-{3-[2-(Cyclohexyl-methyl-amino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₅H₅₁F₃N₆O₃S₂, 724.34; m/z found, 725.5 [M+H]⁺. ¹H NMR (CDCl₃): 7.89 (s, 1H), 7.72 (d, J=8.2, 1H), 7.48 (d, J=8.2, 1H), 4.53 (s, 2H), 4.26-4.18 (m, 1H), 4.19-4.13 (m, 2H), 3.71-3.62 (m, 4H), 3.56-3.48 (m, 1H), 3.44-3.27 (m, 4H), 3.22-3.08 (m, 4H), 2.98-2.88 (m, 5H), 2.88-2.83 (m, 2H), 2.76 (s, 3H), 2.44-2.38 (m, 4H), 2.34-2.15 (m, 2H), 2.08-2.00 (m, 3H), 2.00-1.93 (m, 1H), 1.94-1.83 (m, 4H), 1.75-1.68 (m, 1H), 1.44-1.28 (m, 4H), 1.18-1.07 (m, 1H).

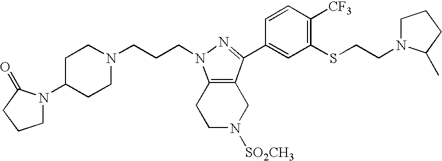

### Example 28

### 1-{1-[3-(5-Methanesulfonyl-3-{3-[2-(2-methyl-pyrrolidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₇F₃N₆O₃S₂, 696.31; m/z found, 697.5 [M+H]⁺. ¹H NMR: 7.86 (s, 1H), 7.75 (d, J=8.2, 1H), 7.65 (d, J=8.2, 1H), 4.47 (s, 2H), 4.17 (t, J=6.5, 2H), 4.03-3.97 (m, 1H), 3.71-3.67 (m, 1H), 3.61-3.51 (m, 6H), 3.47-3.45 (m, 1H), 3.44-3.38 (m, 2H), 3.34-3.30 (m, 2H), 3.16-3.10 (m, 3H), 3.03-2.98 (m, 2H), 2.89 (s, 3H), 2.87-2.85 (m, 2H), 2.29-2.19 (m, 5H), 2.05-1.98 (m, 1H), 1.98-1.84 (m, 7H), 1.66-1.60 (m, 1H), 1.32 (s, 3H).

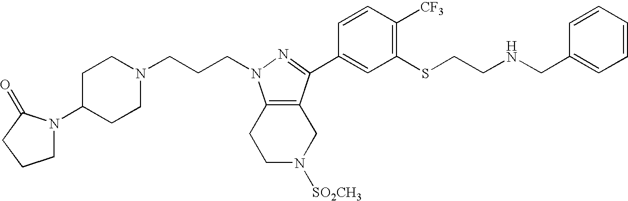

### Example 29

### 1-[1-(3-{3-[3-(2-Benzylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₅H₄₅F₃N₆O₃S₂, 718.29; m/z found, 719.5 [M+H]⁺. ¹H NMR (CDCl₃): 7.90 (s, 1H), 7.68 (d, J=8.2, 1H), 7.39-7.32 (m, 6H), 4.51 (s, 2H), 4.13-4.11 (m, 3H), 4.07 (s, 2H), 3.67-3.63 (m, 2H), 3.62-3.57 (m, 2H), 3.31-3.26 (m, 4H), 3.15-3.10 (m, 4H), 3.01-2.97 (m, 2H), 2.88 (s, 3H), 2.85-2.81 (m, 2H), 2.37-2.33 (m, 2H), 2.43-2.37 (m, 2H), 2.12-2.06 (m, 2H), 2.03-1.97 (m, 2H), 1.78-1.73 (m, 2H).

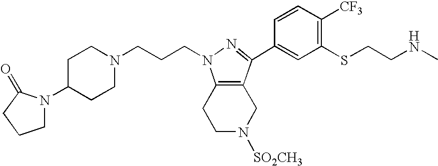

### Example 30

### 1-[1-(3-{5-Methanesulfonyl-3-[3-(2-methylamino-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one

MS (ESI): mass calcd. for C₂₉H₄₁F₃N₆O₃S₂, 642.26; m/z found, 643.4 [M+H]⁺. ¹H NMR: 7.97 (s, 1H), 7.85 (d, J=8.2, 1H), 7.72 (d, J=8.2, 1H), 4.56 (s, 2H), 4.27 (t, J=6.5, 2H), 4.13-4.06 (m, 1H), 3.70-3.63 (m, 4H), 3.40-3.32 (m, 5H), 3.30-3.26 (m, 2H), 3.24-3.20 (m, 2H), 3.13-3.07 (m, 2H), 2.99 (s, 3H), 2.97-2.94 (m, 2H), 2.74 (s, 3H), 2.39-2.32 (m, 4H), 2.06-1.90 (m, 6H).

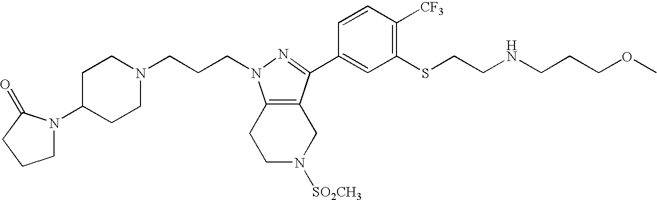

### Example 31

### 1-{1-[3-(5-Methanesulfonyl-3-{3-[2-(3-methoxy-propylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₂H₄₇F₃N₆O₄S₂, 700.31; m/z found, 701.5 [M+H]⁺. ¹H NMR (CDCl₃): 7.93 (s, 1H), 7.71 (d, J=8.2, 1H), 7.41 (d, J=8.2, 1H), 4.52 (s, 2H), 4.25-4.18 (m, 1H), 4.17-4.12 (m, 2H), 3.66 (t, J=5.6, 4H), 3.52 (t, J=5.5, 2H), 3.42-3.38 (m, 2H), 3.36-3.33 (m, 3H), 3.33 (s, 3H), 3.23-3.18 (m, 2H), 3.17-3.12 (m, 4H), 3.03-2.96 (m, 2H), 2.91 (s, 3H), 2.86-2.82 (m, 2H), 2.45-2.36 (m, 4H), 2.24-2.13 (m, 2H), 2.08-2.00 (m, 2H), 2.00-1.97 (m, 2H), 1.88-1.82 (m, 2H).

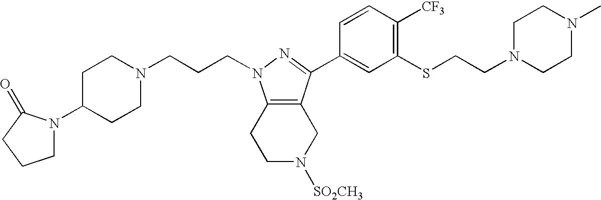

### Example 32

### 1-{1-[3-(5-Methanesulfonyl-3-{3-[2-(4-methyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₈F₃N₇O₃S₂, 711.32; m/z found, 712.5 [M+H]⁺. ¹H NMR (CDCl₃): 7.80 (s, 1H), 7.71 (d, J=8.2, 1H), 7.45 (d, J=8.2, 1H), 4.51 (s, 2H), 4.24-4.16 (m, 3H), 3.69-3.64 (m, 4H), 3.50-3.30 (m, 6H), 3.30-3.22 (m, 6H), 3.15-3.10 (m, 2H), 3.08-3.03 (m, 2H), 2.92 (s, 3H), 2.88-2.84 (m, 4H), 2.82 (s, 3H), 2.40-2.35 (m, 4H), 3.25-3.16 (m, 2H), 2.06-2.00 (m, 2H), 1.88-1.83 (m, 2H).

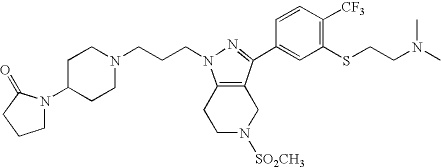

### Example 33

### 1-[1-(3-{3-[3-(2-Dimethylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₀H₄₃F₃N₆O₃S₂, 656.28; m/z found, 657.4 [M+H]⁺. ¹H NMR: 7.88 (s, 1H), 7.73 (d, J=8.2, 1H), 7.60 (d, J=8.2, 1H), 4.46 (s, 2H), 4.16 (t, J=6.5, 2H), 4.03-3.97 (m, 1H), 3.56 (t, J=5.8, 2H), 3.55-3.51 (m, 2H), 3.39-3.36 (m, 2H), 3.27-3.21 (m, 4H), 3.15-3.08 (m, 2H), 3.01-2.95 (m, 2H), 2.89 (s, 3H), 2.84-2.87 (m, 2H), 2.83 (s, 6H), 2.30-2.22 (m, 4H), 1.96-1.88 (m, 4H), 1.83-1.78 (m, 2H).

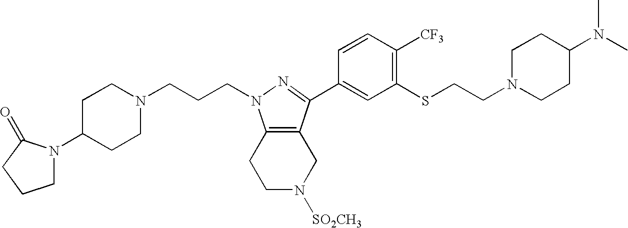

### Example 34

### 1-{1-[3-(3-{3-[2-(4-Dimethylamino-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₅H₅₂F₃N₇O₃S₂, 739.35; m/z found, 740.5 [M+H]⁺. ¹H NMR: 7.89 (s, 1H), 7.81 (d, J=8.2, 1H), 7.74 (d, J=8.2, 1H), 4.56 (s, 2H), 4.25 (t, J=6.4, 2H), 4.10-4.04 (m, 1H), 3.80-3.75 (m, 2H), 3.69-3.61 (m, 4H), 3.50-3.45 (m, 3H), 3.40-3.35 (m, 2H), 3.32-3.29 (m, 2H), 3.25-3.20 (m, 2H), 3.12-3.04 (m, 4H), 2.98 (s, 3H), 2.95-2.92 (m, 2H), 2.89 (s, 6H), 2.37-2.30 (m, 6H), 2.10-1.87 (m, 8H).

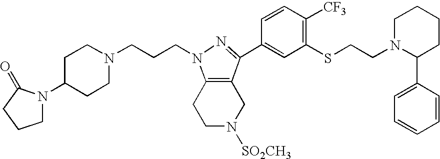

### Example 35

### 1-{1-[3-(5-Methanesulfonyl-3-{3-[2-(2-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₉H₅₁F₃N₆O₃S₂, 772.34; m/z found, 773.5 [M+H]⁺. ¹H NMR: 7.65 (s, 1H), 7.63 (d, J=8.2, 1H), 7.57-7.55 (m, 1H), 7.17-7.16 (m, 2H), 7.14-7.12 (m, 1H), 7.11-7.07 (m, 2H), 4.44 (s, 2H), 4.23-4.19 (m, 2H), 4.12-4.09 (m, 1H), 4.02-3.96 (m, 1H), 3.72-3.63 (m, 2H), 3.59-3.53 (m, 4H), 3.47-3.44 (m, 1H), 3.30-3.28 (m, 2H), 3.18-3.08 (m, 3H), 3.02-2.96 (m, 3H), 2.95 (s, 3H), 2.92-2.55 (m, 3H), 2.30-2.24 (m, 4H), 1.98-1.80 (m, 12H).

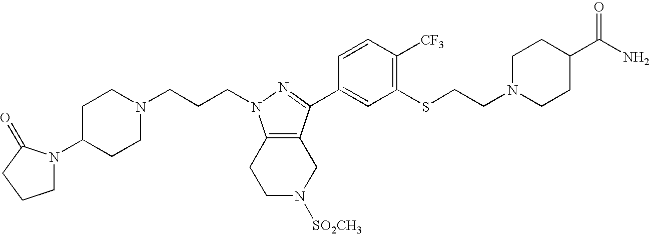

### Example 36

### 1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl}-piperidine-4-carboxylic acid amide

MS (ESI): mass calcd. for C₃₄H₄₈F₃N₇O₄S₂, 739.32; m/z found, 740.54 [M+H]⁺.

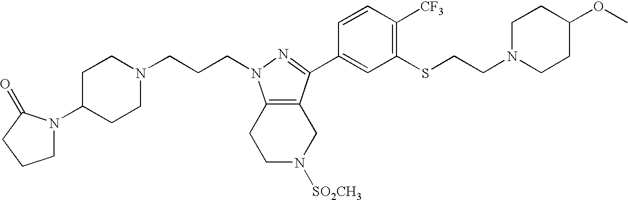

### Example 37

### 1-{1-[3-(5-Methanesulfonyl-3-{3-[2-(4-methoxy-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₉F₃N₆O₄S₂, 726.32; m/z found, 727.5 [M+H]⁺.

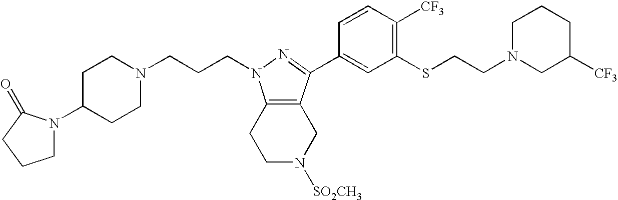

### Example 38

### 1-{1-[3-(5-Methanesulfonyl-3-{4-trifluoromethyl-3-[2-(3-trifluoromethyl-piperidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₆F₆N₆O₃S₂, 764.30; m/z found, 767.5 [M+H]⁺.

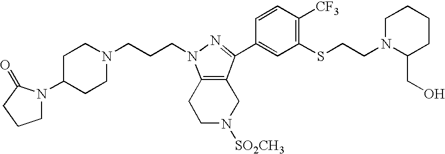

### Example 39

### 1-{1-[3-(3-{3-[2-(2-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₉F₃N₆O₄S₂, 726.32; m/z found, 727.3 [M+H]⁺.

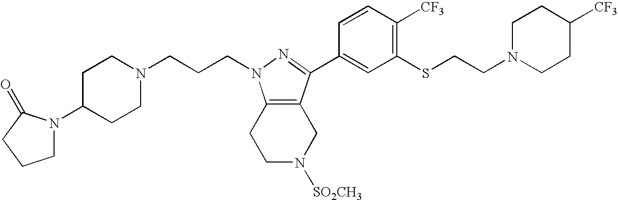

### Example 40

### 1-{1-[3-(5-Methanesulfonyl-3-{4-trifluoromethyl-3-[2-(4-trifluoromethyl-piperidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₆F₆N₆O₃S₂, 764.30; m/z found, 765.4 [M+H]⁺.

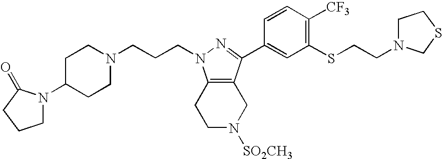

### Example 41

### 1-[1-(3-{5-Methanesulfonyl-3-[3-(2-thiazolidin-3-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one

MS (ESI): mass calcd. for C₃₁H₄₃F₃N₆O₃S₃, 700.25; m/z found, 701.1 [M+H]⁺.

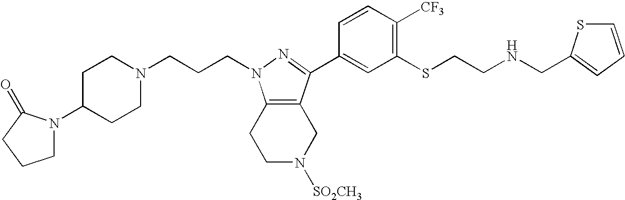

### Example 42

### 1-(1-{3-[5-Methanesulfonyl-3-(3-{2-[(thiophen-2-ylmethyl)-amino]-ethylsulfanyl}-4-trifluoromethyl-phenyl)-4,5,6,7-tetrahydro-pyrazolo[4,3-c]pyridin-1-yl]-propyl}-piperidin-4-yl)-pyrrolidin-2-one

MS (ESI): mass calcd. for C₃₃H₄₃F₃N₆O₃S₃, 724.25; m/z found, 725.4 [M+H]⁺.

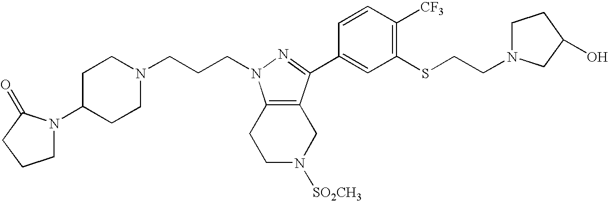

### Example 43

### 1-{1-[3-(3-{3-[2-(3-Hydroxy-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₂H₄₅F₃N₆O₄S₂, 698.29; m/z found, 699.5 [M+H]⁺.

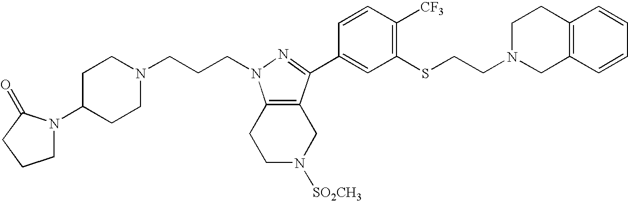

### Example 44

### 1-{1-[3-(3-{3-[2-(3,4-Dihydro-1H-isoquinolin-2-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₇H₄₇F₃N₆O₃S₂, 744.31; m/z found, 745.5 [M+H]⁺.

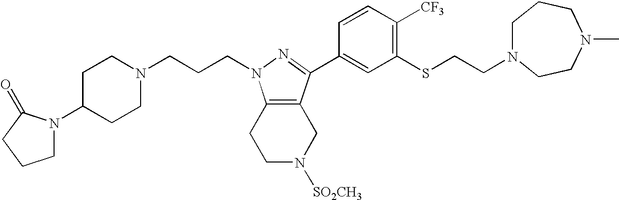

### Example 45

### 1-{1-[3-(5-Methanesulfonyl-3-{3-[2-(4-methyl-[1,4]diazepan-1-yl)-ethylsulfanyl]-4-trifluoromethyl-phenyl-4,5,6,7-tetrahydro-pyrazolo[4,3-c]pyridin-1-yl)-propyl]-piperidin-4-yl}-pyrrolidin-2-one

MS (ESI): mass calcd. for C₃₄H₅₀F₃N₇O₃S₂, 725.34; m/z found, 726.5 [M+H]⁺.

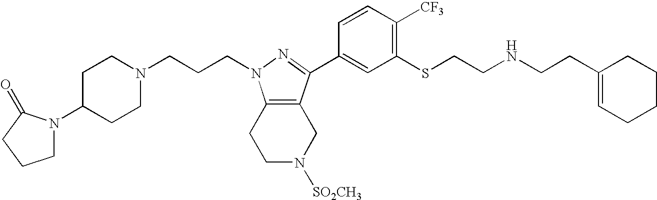

### Example 46

### 1-{1-[3-(3-{3-[2-(2-Cyclohex-1-enyl-ethylamino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₆H₅₁F₃N₆O₃S₂, 736.34; m/z found, 737.5 [M+H]⁺.

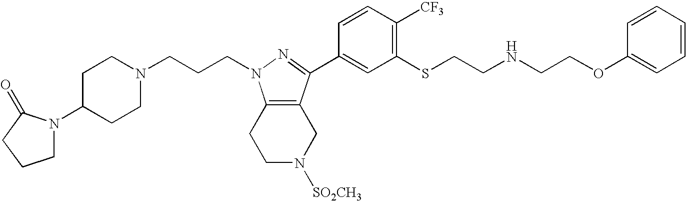

### Example 47

### 1-{1-[3-(5-Methanesulfonyl-3-{3-[2-(2-phenoxy-ethylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₆H₄₇F₃N₆O₄S₂, 748.31; m/z found, 749.5 [M+H]⁺.

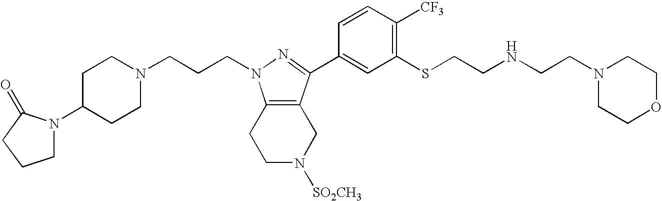

### Example 48

### 1-{1-[3-(5-Methanesulfonyl-3-{3-[2-(2-morpholin-4-yl-ethylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₄H₅₀F₃N₇O₄S₂, 741.33; m/z found, 742.5 [M+H]⁺.

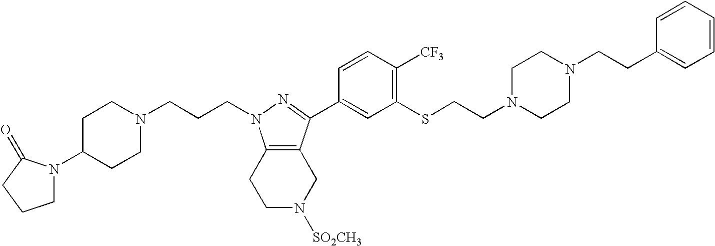

### Example 49

### 1-{1-[3-(5-Methanesulfonyl-3-{3-[2-(4-phenethyl-piperazin-1-yl)-ethylsulfanyl]-4-trifluoromethyl-phenyl-4,5,6,7-tetrahydro-pyrazolo[4,3-c]pyridin-1-yl)-propyl]-piperidin-4-yl}-pyrrolidin-2-one

MS (ESI): mass calcd. for C₄₀H₅₄F₃N₇O₃S₂, 801.37; m/z found, 802.5 [M+H]⁺.

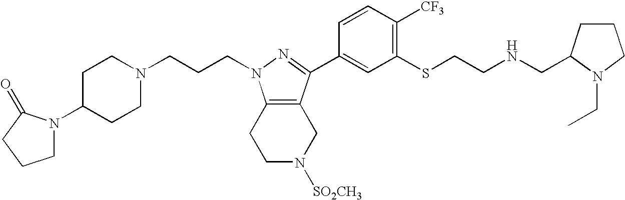

### Example 50

### 1-(1-{3-[3-(3-{2-[(1-Ethyl-pyrrolidin-2-ylmethyl)-amino]-ethylsulfanyl}-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one

MS (ESI): mass calcd. for C₃₅H₅₂F₃N₇O₃S₂, 739.35; m/z found, 740.5 [M+H]⁺.

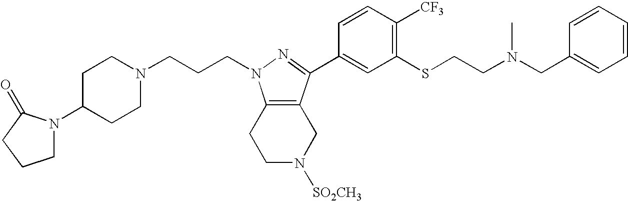

### Example 51

### 1-{1-[3-(3-{3-[2-(Benzyl-methyl-amino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₆H₄₇F₃N₆O₃S₂, 732.21; m/z found, 733.5 [M+H]⁺.

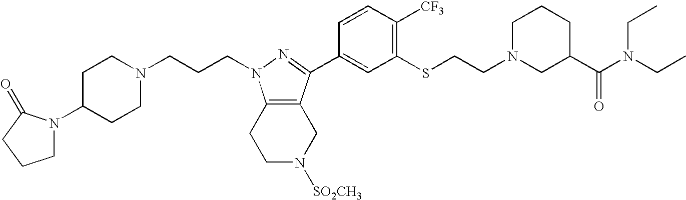

### Example 52

### 1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl}-piperidine-3-carboxylic acid diethylamide

MS (ESI): mass calcd. for C₃₈H₅₆F₃N₇O₄S₂, 795.38; m/z found, 796.5 [M+H]⁺.

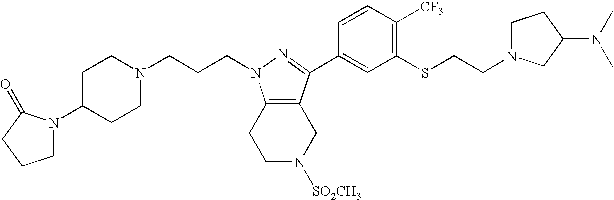

### Example 53

### 1-{1-[3-(3-{3-[2-(3-Dimethylamino-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₄H₅₀F₃N₇O₃S₂, 725.34; m/z found, 726.5 [M+H]⁺.

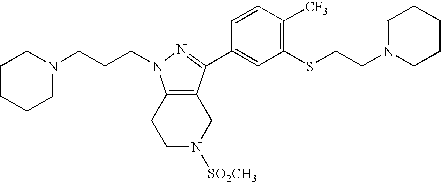

### Example 54

### 5-Methanesulfonyl-3-[3-(2-piperidin-1-yl-ethylsulfanyl)-4-trifluoromethyl-phenyl]-1-(3-piperidin-1-yl-propyl)-4,5,6,7-tetrahydro-1H-pyrazolo[4,3-c]pyridine

A. Methanesulfonic acid 2-[5-[1-(2-[1,3]dioxolan-2-yl-ethyl)-5-methanesulfonyl-4,5,6,7-tetrahydro-1H-pyrazolo[4,3-c]pyridin-3-yl]-2-trifluoromethyl-phenylsulfanyl]-ethyl ester. To a solution of 2-{5-[1-(2-[1,3]dioxolan-2-yl-ethyl)-5-methanesulfonyl-4,5,6,7-tetrahydro-1H-pyrazolo[4,3-c]pyridin-3-yl]-2-trifluoromethyl-phenylsulfanyl}-ethanol (1.4 g, 2.7 mmol) in CH₂Cl₂ (13 mL) was added pyridine (0.5 mL, 6.1 mmol) and MsCl (0.31 mL, 4.0 mmol). The reaction was stirred at rt for 18 h and additional MsCl (0.1 mL) was added to the mixture. After another 24 h, the mixture was poured into H₂O (20 mL) and the layers were separated. The aqueous layer was extracted with CH₂Cl₂ (3×, 10 mL). The combined organic layers were dried (Na₂SO₄) and concentrated to a light orange solid, which was used without further purification. MS (ESI): mass calcd. for C₂₂H₂₈F₃N₃O₇S₃, 599.10; m/z found, 600.3 [M+H]⁺.

B. 1-(2-[1,3]Dioxolan-2-yl-ethyl)-5-methanesulfonyl-3-[3-(2-piperidin-1-yl-ethylsulfanyl)-4-trifluoromethyl-phenyl]-4,5,6,7-tetrahydro-1H-pyrazolo[4,3-c]pyridine To a solution of methanesulfonic acid 2-{5-[1-(2-[1,3]dioxolan-2-yl-ethyl)-5-methanesulfonyl-4,5,6,7-tetrahydro-1H-pyrazolo[4,3-c]pyridin-3-yl]-2-trifluoromethyl-phenylsulfanyl}-ethyl ester (1.6 g, 2.7 mmol) in 1:2 dichloroethane/EtOH (15 mL) was added piperidine (5.3 mL, 54.0 mmol). The reaction mixture was heated to 60° C. for 14 h. After concentration, the resulting residue was re-dissolved in 1:1 CH₂Cl₂/H₂O (50 mL). The layers were separated, and the aqueous layer was extracted with CH₂Cl₂ (3×, 20 mL). The combined organic layers were washed with brine (20 mL), dried (Na₂SO₄) and concentrated to an orange solid, which was used without additional purification. MS (ESI): mass calcd. for C₂₆H₃₅F₃N₄O₄S₂, 588.21; m/z found, 589.2 [M+H]⁺.

C. 5-Methanesulfonyl-3-[3-(2-piperidin-1-yl-ethylsulfanyl)-4-trifluoromethyl-phenyl]-1-(3-piperidin-1-yl-propyl)-4,5,6,7-tetrahydro-1H-pyrazolo[4,3-c]pyridine. This compound was prepared using the methods outlined in Example 1, Steps E and F, substituting 1-(2-[1,3]dioxolan-2-yl-ethyl)-5-methanesulfonyl-3-[3-(2-piperidin-1-yl-ethylsulfanyl)-4-trifluoromethyl-phenyl]-4,5,6,7-tetrahydro-1H-pyrazolo[4,3-c]pyridine for 2-{5-[1-(2-[1,3]dioxolan-2-yl-ethyl)-5-methanesulfonyl-4,5,6,7-tetrahydro-1H-pyrazolo[4,3-c]pyridin-3-yl]-2-trifluoromethyl-phenylsulfanyl}-ethanol and piperidine for 4-(N-2-pyrrolidinone)piperidine. MS (ESI): mass calcd. for C₂₉H₄₂F₃N₅O₂S₂, 613.27; m/z found, 614.4 [M+H]⁺. ¹H NMR: 7.83 (s, 1H), 7.72 (d, J=8.2, 1H), 7.63 (d, J=8.2, 1H), 4.47 (s, 2H), 4.15 (t, J=6.7, 2H), 3.57 (t, J=5.8, 2H), 3.50-3.42 (m, 4H), 3.39-3.35 (m, 2H), 3.27-3.23 (m, 2H), 3.10-3.06 (m, 2H), 2.89 (s, 3H), 2.88-2.79 (m, 6H), 2.26-2.20 (m, 2H), 1.87-1.81 (m, 4H), 1.75-1.68 (m, 3H), 1.68-1.59 (m, 3H), 1.44-1.35 (m, 2H).

Examples 55-60 were prepared according to the methods described in Example 54, with the appropriate substituent changes.

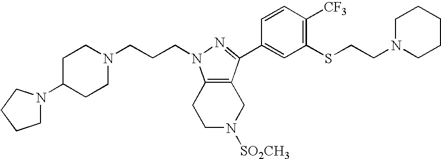

### Example 55

### 5-Methanesulfonyl-3-[3-(2-piperidin-1-yl-ethylsulfanyl)-4-trifluoromethyl-phenyl]-1-[3-(4-pyrrolidin-1-yl-piperidin-1-yl)-propyl]-4,5,6,7-tetrahydro-1H-pyrazolo[4,3-c]pyridine

MS (ESI): mass calcd. for C₃₃H₄₉F₃N₆O₂S₂, 682.33; m/z found, 683.5 [M+H]⁺. ¹H NMR: 7.83 (s, 1H), 7.72 (d, J=8.2, 1H), 7.63 (d, J=8.2, 1H), 4.47 (s, 2H), 4.15 (t, J=6.6, 2H), 3.68-3.62 (m, 2H), 3.59-3.55 (m, 3H), 3.50-3.45 (m, 2H), 3.38-3.34 (m, 3H), 3.27-3.23 (m, 3H), 3.16-3.11 (m, 2H), 3.08-2.94 (m, 4H), 2.90 (s, 3H), 2.89-2.83 (m, 4H), 2.36-2.30 (m, 2H), 2.29-2.22 (m, 2H), 2.11-2.01 (m, 2H), 2.01-1.87 (m, 4H), 1.87-1.81 (m, 2H), 1.75-1.60 (m, 3H), 1.44-1.35 (m, 1H).

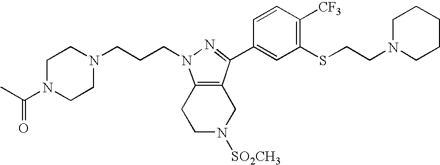

### Example 56

### 1-[4-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperazin-1-yl]-ethanone

MS (ESI): mass calcd. for C₃₀H₄₃F₃N₆O₃S₂, 656.28; m/z found, 657.4 [M+H]⁺. ¹H NMR: 7.83 (s, 1H), 7.73 (d, J=8.2, 1H), 7.64 (d, J=8.2, 1H), 4.47 (s, 2H), 4.17 (t, J=6.6, 2H), 3.57 (t, J=5.8, 2H), 3.50-3.40 (m, 3H), 3.39-3.35 (m, 3H), 3.28-3.24 (m, 3H), 3.22-3.20 (m, 4H), 3.19-3.15 (m, 3H), 2.89 (s, 3H), 2.89-2.84 (m, 4H), 2.30-2.24 (m, 2H), 2.03 (s, 3H), 1.87-1.82 (m, 2H), 1.75-1.60 (m, 3H), 1.44-1.36 (m, 1H).

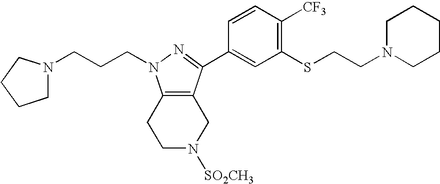

### Example 57

### 5-Methanesulfonyl-3-[3-(2-piperidin-1-yl-ethylsulfanyl)-4-trifluoromethyl-phenyl]-1-(3-pyrrolidin-1-yl-propyl)-4,5,6,7-tetrahydro-1H-pyrazolo[4,3-c]pyridine

MS (ESI): mass calcd. for C₂₈H₄₀F₃N₅O₂S₂, 599.26; m/z found, 600.3 [M+H]⁺. ¹H NMR: 7.92 (s, 1H), 7.79 (d, J=8.2, 1H), 7.66 (d, J=8.2, 1H), 4.56 (s, 2H), 4.25 (t, J=6.7, 2H), 3.74-3.65 (m, 5H), 3.62-3.56 (m, 2H), 3.52-3.47 (m, 2H), 3.38-3.27 (m, 5H), 3.16-3.05 (m, 4H), 2.98-2.93 (m, 2H), 2.38-2.31 (m, 2H), 2.19-2.11 (m, 2H), 2.08-1.90 (m, 4H), 1.85-1.75 (m, 3H), 1.66-1.46 (m, 2H).

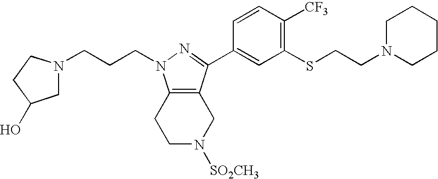

### Example 58

### 1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-pyrrolidin-3-ol.

MS (ESI): mass calcd. for C₂₈H₄₀F₃N₅O₂S₂, 615.25; m/z found, 616.3 [M+H]⁺. ¹H NMR: 7.82 (s, 1H), 7.73 (d, J=8.2, 1H), 7.66-7.62 (m, 1H), 4.47 (s, 2H), 4.46-4.42 (m, 1H), 4.16 (t, J=6.6, 2H), 3.72-3.61 (m, 1H), 3.59-3.56 (m, 3H), 3.50-3.43 (m, 4H), 3.39-3.35 (m, 3H), 3.28-3.23 (m, 3H), 3.14-2.99 (m, 2H), 2.89 (s, 3H), 2.89-2.84 (m, 4H), 2.26-2.19 (m, 2H), 2.04-1.96 (m, 1H), 1.87-1.81 (m, 2H), 1.76-1.69 (m, 1H), 1.69-1.60 (m, 2H), 1.44-1.35 (m, 1H).

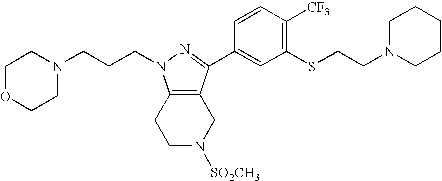

### Example 59

### 5-Methanesulfonyl-1-(3-morpholin-4-yl-propyl)-3-[3-(2-piperidin-1-yl-ethylsulfanyl)-4-trifluoromethyl-phenyl]-4,5,6,7-tetrahydro-1H-pyrazolo[4,3-c]pyridine

MS (ESI): mass calcd. for C₂₈H₄₀F₃N₅O₃S₂, 615.25; m/z found, 616.3 [M+H]⁺. ¹H NMR: 7.85 (s, 1H), 7.72 (d, J=8.2, 1H), 7.61 (d, J=8.2, 1H), 4.48 (s, 2H), 4.18 (t, J=6.7, 2H), 3.98-3.93 (m, 2H), 3.73-3.66 (m, 2H), 3.60-3.57 (m, 2H), 3.53-3.48 (m, 2H), 3.46-3.38 (m, 4H), 3.29-3.23 (m, 2H), 3.21-3.16 (m, 2H), 3.10-3.05 (m, 2H), 2.92 (s, 3H), 2.90-2.85 (m, 2H), 2.32-2.26 (m, 2H), 1.88-1.82 (m, 2H), 1.76-1.65 (m, 3H), 1.59-1.53 (m, 1H), 1.53-1.38 (m, 2H).

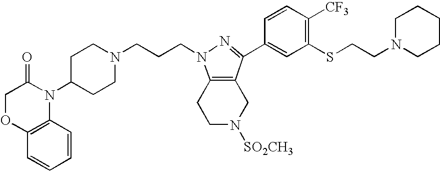

### Example 60

### 4-[1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-4H-benzo[1,4]oxazin-3-one

MS (ESI): mass calcd. for C₃₇H₄₇F₃N₆O₄S₂, 760.31; m/z found, 761.3 [M+H]⁺. ¹H NMR: 7.91 (s, 1H), 7.80 (d, J=8.2, 1H), 7.71 (d, J=8.2, 1H), 7.29 (d, J=7.6, 1H), 7.07-6.98 (m, 3H), 4.55 (s, 2H), 4.44 (s, 2H), 4.44-4.37 (m, 1H), 4.26-4.22 (m, 2H), 3.71-3.63 (m, 4H), 3.57-3.51 (m, 2H), 3.46-3.42 (m, 2H), 3.35-3.30 (m, 2H), 3.24-3.19 (m, 2H), 3.18-3.11 (m, 2H), 3.05-2.98 (m, 2H), 2.97 (s, 3H), 2.96-2.91 (m, 4H), 2.37-2.32 (m, 2H), 2.08-2.03 (m, 2H), 1.93-1.88 (m, 2H), 1.82-1.68 (m, 3H), 1.50-1.42 (m, 1H).

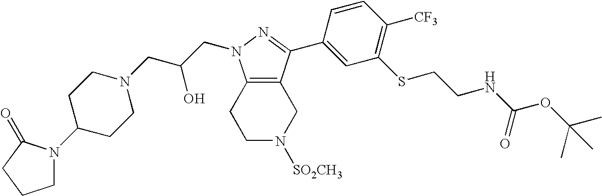

### Example 61

### {2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-carbamic acid tert-butyl ester

A. {2-[5-(5-Methanesulfonyl-4,5,6,7-tetrahydro-1H-pyrazolo[4,3-c]pyridin-3-yl)-2-trifluoromethyl-phenylsulfanyl]-ethyl}-carbamic acid tert-butyl ester. The title compound was prepared using methods similar to those described in Example 1, Steps A-C, with the appropriate substituent changes. MS (ESI): mass calcd. for C₂₁H₂₇F₃N₄O₄S₂, 520.1; m/z found, 465.2 [M-tButyl]⁺. ¹H NMR (acetone-d₆): 8.04 (s, 1H), 7.78 (d, J=8.2, 1H), 7.56 (d, J=8.0, 1H), 4.63 (br s, 2H), 3.63 (t, J=5.8, 2H), 3.37-3.35 (m, 2H), 3.28-3.24 (m, 3H), 2.94 (s, 3H), 2.92-2.90 (m, 3H), 1.43 (s, 9H).

B. {2-[5-(5-Methanesulfonyl-1-oxiranylmethyl-4,5,6,7-tetrahydro-1H-pyrazolo[4,3-c]pyridin-3-yl)-2-trifluoromethyl-phenylsulfanyl]-ethyl}-carbamic acid tert-butyl ester. To a stirred solution of {2-[5-(5-methanesulfonyl-4,5,6,7-tetrahydro-1H-pyrazolo[4,3-c]pyridin-3-yl)-2-trifluoromethyl-phenylsulfanyl]-ethyl}-carbamic acid tert-butyl ester (450 mg, 0.7 mmol) and epichlorohydrin (656 mg, 7.0 mmol) in DMF (5.0 mL) was added Cs₂CO₃ (346 mg, 1.1 mmol). The reaction mixture was stirred at rt under N₂ for 18 h. To the mixture was added a solution of cold satd. aq. NH₄Cl (100 mL) and the product was extracted with CH₂Cl₂ (3×, 100 mL). The combined extracts were dried (Na₂SO₄) and concentrated to give a light brown liquid, which was purified through reverse phase HPLC to give the desired product as a white solid (195 mg, 40%). MS (ESI): mass calcd. for C₂₄H₃₁F₃N₄O₅S₂, 576.2; m/z found, 521.3 [M-tButyl]⁺. ¹H NMR: 7.89 (s, 1H), 7.73 (d, J=8.2, 1H), 7.54 (d, J=8.2, 1H), 4.57-4.53 (m, 3H), 4.18-4.13 (dd, J=15.2, 5.8, 1H), 3.69-3.57 (m, 2H), 3.37-3.29 (m, 2H), 3.19-3.16 (m, 2H), 2.94 (s, 3H), 2.82 (t, J=4.5, 1H), 2.77 (t, J=6.9, 1H), 2.54-2.52 (m, 1H), 1.49-1.39 (m, 3H), 1.39 (s, 9H).

C. {2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-carbamic acid tert-butyl ester. To a solution of {2-[5-(5-methanesulfonyl-1-oxiranylmethyl-4,5,6,7-tetrahydro-1H-pyrazolo[4,3-c]pyridin-3-yl)-2-trifluoromethyl-phenylsulfanyl]-ethyl}-carbamic acid tert-butyl ester (816 mg, 1.1 mmol) in EtOH (10.0 mL) was added 1-piperidin-4-yl-pyrrolidin-2-one (238 mg, 1.4 mmol). The reaction mixture was stirred at 90° C. under N₂ for 18 h. The reaction mixture was concentrated to give white foam, which was purified (SiO₂; 1:9:90 satd. NH₃ in MeOH/MeOH/CH₂Cl₂) to give the desired product as a white solid (768 mg, 88%). MS (ESI): mass calcd. for C₃₃H₄₇F₃N₆O₆S₂, 744.3; m/z found, 745.5 [M+H]⁺. ¹H NMR (acetone-d₆): 7.91 (s, 1H), 7.75-7.74 (d J=8.2, 1H), 6.23 (s, 1H), 4.61 (br s, 2H), 4.27-4.24 (dd, J=13.4, 3.2, 1H), 4.18-4.12 (m, 2H), 4.09 (t, J=6.7, 1H), 3.82-3.75 (m, 1H), 3.67-3.57 (m, 2H), 3.37-3.32 (m, 4H), 3.22 (t, J=7.0, 2H), 3.09-2.94 (m, 4H), 2.93 (s, 3H), 2.48-2.39 (m, 2H), 2.18 (t, J=8.0, 3H), 2.17-2.14 (m, 1H), 2.12-2.07 (td, J=12.0, 2.2, 1H), 1.96-1.90 (m, 2H), 1.76-1.68 (m, 2H), 1.54-1.51 (m, 2H), 1.37 (s, 9H).

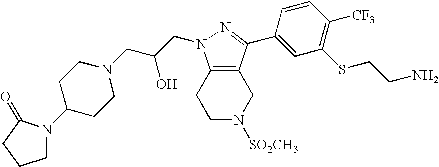

### Example 62

### 1-[1-(3-{3-[3-(2-Amino-ethylsulfanyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-pyrrolidin-2-one

To a stirred solution of {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-carbamic acid tert-butyl ester (132 mg, 0.17 mmol) in CH₂Cl₂ (363 μL) was added trifluoroacetic acid (262 mg, 2.3 mmol) and triethylsilane (51 mg, 0.44 mmol). The reaction mixture was stirred at rt for 1.5 h. The reaction mixture was concentrated to give a clear oil, which was purified through reverse phase HPLC to give the desired product as a white solid (76 mg, 57%). MS (ESI): mass calcd. for C₂₈H₃₉F₃N₆O₄S₂, 644.2; m/z found, 645.4 [M+H]⁺. ¹H NMR: 7.91 (s, 1H), 7.75-7.74 (d J=8.2, 1H), 6.23 (s, 1H), 4.55 (br s, 2H), 4.51-4.50 (br m, 1H), 4.27-4.19 (m, 2H), 4.17-4.09 (m, 1H), 3.79-3.60 (m, 5H), 3.41 (t, J=6.7, 2H), 3.34 (t, J=7.0, 2H), 3.27-3.10 (m, 6H), 3.01-2.99 (m, 2H), 2.97 (s, 3H), 2.38 (t, J=8.0, 2H), 2.18-2.11 (m, 2H), 2.08-2.00 (m, 3H), 1.94-1.89 (m, 2H).

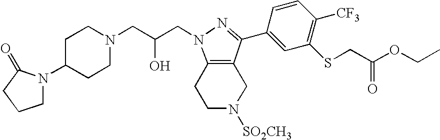

### Example 63

### [5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid ethyl ester

This compound was prepared using the methods outlined in Example 61, with the appropriate substituent changes. The final product was isolated as a mixture of regioisomers, as determined by NMR. MS (ESI): mass calcd. for C₃₀H₄₀F₃N₅O₆S₂, 687.24; m/z found, 688.5 [M+H]⁺. ¹H NMR: 7.93 (s, 1H), 7.83 (d, J=8.3, 2H), 7.75 (d, J=8.2, 1H), 7.66 (d, J=8.3, 1H), 7.50 (d, J=8.0, 1H), 4.55 (s, 2H), 4.48 (br s, 2H), 4.36-4.16 (m, 5H), 4.15-4.06 (m, 6H), 3.98-3.88 (m, 2H), 3.86 (s, 2H), 3.76-3.54 (m, 9H), 3.70 (s, 3H), 3.46-3.36 (m, 5H), 3.27-3.06 (m, 7H), 3.02-2.88 (m, 5H), 2.99 (s, 3H), 2.92 (s, 3H), 2.42-2.34 (m, 5H), 2.18-2.08 (m, 2H), 2.08-1.98 (m, 7H), 1.96-1.84 (m, 4H), 1.15 (t, J=7.1, 3H).

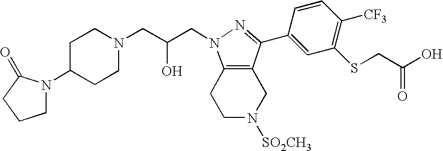

### Example 64

### [5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid

To a solution of [5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid ethyl ester (0.41 g, 0.59 mmol) in IPA (0.70 mL) was added a solution of KOH (0.07 g, 1.2 mmol) in H₂O (1.3 mL). The reaction mixture stirred for 24 h at rt. IPA was removed under reduced pressure. The aqueous layer was frozen and lyophilized to afford a white powder, which was used without further purification. MS (ESI): mass calcd. for C₂₈H₃₆F₃N₅O₅S₂, 659.74; m/z found, 660.5 [M+H]⁺. ¹H NMR: 7.74 (s, 1H), 7.67 (d, J=8.0, 1H), 7.57 (d, J=8.5, 1H), 4.56 (s, 2H), 4.23-4.16 (m, 2H), 4.07-4.01 (m, 1H), 3.94-3.89 (m, 1H), 3.88-3.79 (m, 1H), 3.69 (s, 2H), 3.68-3.58 (m, 2H), 3.44-3.38 (m, 2H), 3.44-3.38 (m, 2H), 3.05-2.99 (m, 2H), 3.01 (s, 3H), 2.48-2.44 (m, 2H), 2.35 (t, J=8.1, 2H), 2.19-2.12 (m, 2H), 2.03-1.96 (m, 2H), 1.81-1.70 (m, 2H), 1.63-1.54 (m, 2H).

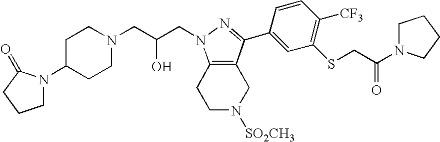

### Example 65

### 1-[1-(2-Hydroxy-3-{5-methanesulfonyl-3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one

To a solution of [5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid (0.04 g, 0.06 mmol) in DMF (0.30 mL) was added EDC (0.02 g, 0.09 mmol), HOBt (0.01 g, 0.09 mmol), pyrrolidine (0.01 g, 0.09 mmol) and TEA (0.01 g, 0.18 mmol). The reaction was monitored by HPLC, and additional coupling reagents were added when reaction progress was slow. The product was isolated by reverse phase HPLC to yield a white solid (26.2 mg, 60%). MS (ESI): mass calcd. for C₃₂H₄₃F₃N₆O₅S₂, 712.27; m/z found, 713.4 [M+H]⁺. ¹H NMR (CDCl₃): 8.02 (s, 1H), 7.64 (d, J=8.3, 1H), 7.61 (d, J=8.6, 1H), 4.63 (s, 2H), 4.18-4.13 (m, 1H), 4.12-4.06 (m, 1H), 4.02-3.93 (m, 2H), 3.71 (s, 2H), 3.69-3.59 (m, 2H), 3.51 (t, J=6.8, 2H), 3.44 (t, J=6.9, 2H), 3.31 (t, J=7.0, 2H), 3.03-2.96 (m, 2H), 2.96 (s, 3H), 2.93 (s, 1H), 2.92-2.83 (m, 2H), 2.86 (s, 1H), 2.47-2.38 (m, 2H), 2.38-2.33 (m, 2H), 2.17-2.08 (m, 1H), 2.02-1.92 (m, 4H), 1.87-1.80 (m, 2H), 1.75-1.69 (m, 1H), 1.67-1.61 (m, 3H).

Examples 66-74 were prepared using methods similar to those described in Example 65, with the appropriate substituent changes.

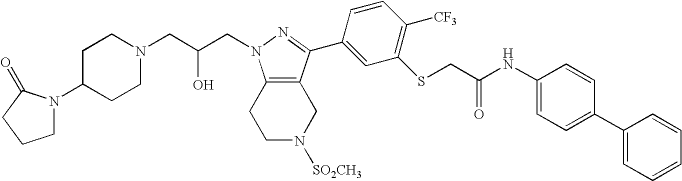

### Example 66

### N-Biphenyl-4-yl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide

MS (ESI): mass calcd. for C₄₀H₄₅F₃N₆O₅S₂, 810.28; m/z found, 811.5 [M+H]⁺. ¹H NMR (CDCl₃): 8.61 (s, 1H), 7.86 (s, 1H), 7.73 (d, J=8.2, 1H), 7.57 (d, J=8.4, 2H), 7.53 (d, J=8.2, 2H), 7.45 (d, J=8.3, 2H), 7.42 (t, J=7.6, 2H), 7.38-7.30 (m, 2H), 4.58-4.46 (m, 3H), 4.26-4.18 (m, 2H), 4.14-4.08 (m, 1H), 4.02-3.86 (m, 2H), 3.64 (t, J=5.8, 2H), 3.62-3.51 (m, 2H), 3.34-3.25 (m, 2H), 3.23-3.17 (m, 1H), 3.16-3.09 (m, 2H), 3.08-2.96 (m, 2H), 2.89 (s, 3H), 2.87-2.80 (m, 1H), 2.41-2.36 (m, 2H), 2.28-2.18 (m, 1H), 2.16-2.06 (m, 1H), 2.04-1.97 (m, 3H), 1.66-1.60 (m, 2H).

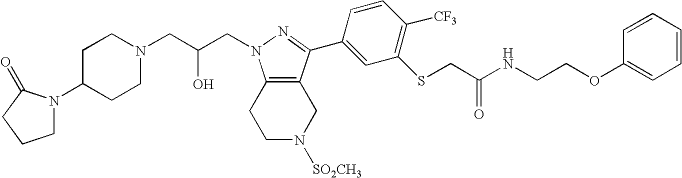

### Example 67

### 2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-(2-phenoxy-ethyl)-acetamide

MS (ESI): mass calcd. for C₃₆H₄₅F₃N₆O₆S₂, 778.28; m/z found, 779.5 [M+H]⁺. ¹H NMR (CDCl₃): 7.60 (s, 1H), 7.54 (d, J=8.1, 1H), 7.26-7.24 (m, 1H), 7.23-7.17 (m, 3H), 6.95-6.90 (m, 1H), 6.69-6.65 (m, 2H), 4.62-4.54 (m, 1H), 4.50-4.48 (m, 2H), 4.34-4.08 (m, 3H), 3.90-3.84 (m, 2H), 3.83-3.78 (m, 3H), 3.70-3.60 (m, 5H), 3.42-3.31 (m, 3H), 3.28-3.14 (m, 3H), 3.07-3.00 (m, 1H), 2.97 (s, 3H), 2.92-2.82 (m, 1H), 2.42 (t, J=8.0, 2H), 2.34-2.20 (m, 3H), 2.07-2.02 (m, 2H), 1.90-1.78 (m, 2H).

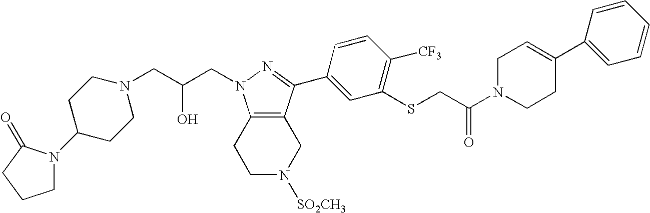

### Example 68

### 1-{1-[2-Hydroxy-3-(5-methanesulfonyl-3-{3-[2-oxo-2-(4-phenyl-3,6-dihydro-2H-pyridin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₉H₄₇F₃N₆O₅S₂, 800.30; m/z found, 801.4 [M+H]⁺.

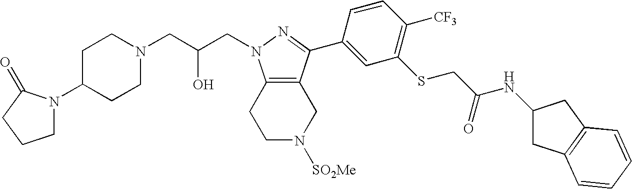

### Example 69

### 2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-indan-2-yl-acetamide

MS (ESI): mass calcd. for C₃₇H₄₅F₃N₆O₅S₂, 774.28; m/z found, 775.5 [M+H]⁺.

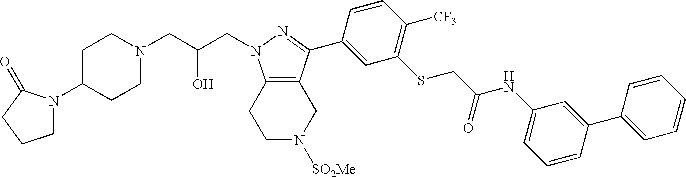

### Example 70

### N-Biphenyl-3-yl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide

MS (ESI): mass calcd. for C₄₀H₄₅F₃N₆O₅S₂, 810.28; m/z found, 811.4 [M+H]⁺.

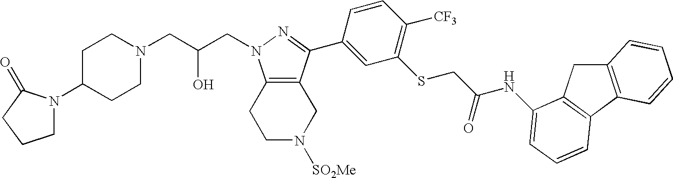

### Example 71

### N-(9H-Fluoren-1-yl)-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide

MS (ESI): mass calcd. for C₄₁H₄₅F₃N₆O₅S₂, 822.28; m/z found, 823.4 [M+H]⁺.

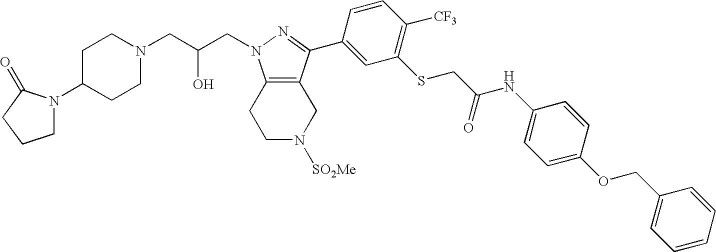

### Example 72

### N-(4-Benzyloxy-phenyl)-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide

MS (ESI): mass calcd. for C₄₁H₄₇F₃N₆O₆S₂, 840.30; m/z found, 841.5 [M+H]⁺.

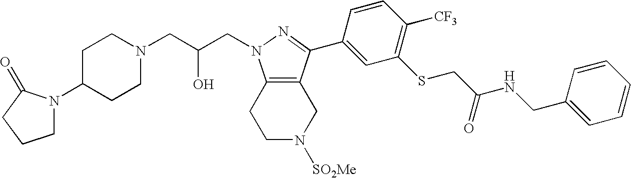

### Example 73

### N-Benzyl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide

MS (ESI): mass calcd. for C₃₅H₄₃F₃N₆O₅S₂, 748.27; m/z found, 749.4 [M+H]⁺.

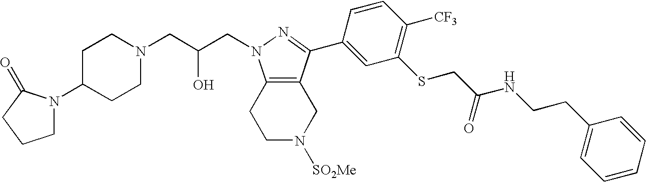

### Example 74

### 2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-phenethyl-acetamide

MS (ESI): mass calcd. for C₃₆H₄₅F₃N₆O₅S₂, 762.28; m/z found, 763.5 [M+H]⁺.

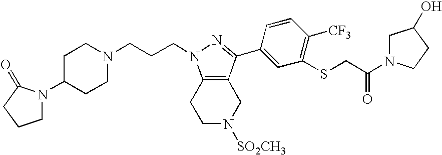

### Example 75

### 1-{1-[3-(3-{3-[2-(3-Hydroxy-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

A. [5-(5-Methanesulfonyl-4,5,6,7-tetrahydro-1H-pyrazolo[4,3-c]pyridin-3-yl)-2-trifluoromethyl-phenylsulfanyl]-acetic acid ethyl ester. This compound was prepared using methods similar to those described in Example 1, Step C, with the appropriate substituent changes. MS (ESI): mass calcd. for C₁₈H₂₀F₃N₃O₄S₂, 463.08; m/z found, 464.1 [M+H]⁺. ¹H NMR (CDCl₃): 7.85 (s, 1H), 7.67 (d, J=8.2, 1H), 7.48 (d, J=7.9, 1H), 4.58 (s, 2H), 4.17 (dd, J=14.5, 7.4, 2H), 3.68 (s, 2H), 3.65 (t, J=5.8, 2H), 2.95 (s, 3H), 2.94-2.88 (m, 2H), 1.24-1.19 (m, 3H).

B. {5-[1-(2-[1,3]Dioxolan-2-yl-ethyl)-5-methanesulfonyl-4,5,6,7-tetrahydro-1H-pyrazolo[4,3-c]pyridin-3-yl]-2-trifluoromethyl-phenylsulfanyl]-acetic acid ethyl ester. This compound was prepared using methods similar to those described in Example 1, Step D. MS (ESI): mass calcd. for C₂₃H₂₈F₃N₃O₆S₂, 563.14; m/z found, 564.1 [M+H]⁺. ¹H NMR: 7.91 (s, 1H), 7.75 (d, J=8.2, 1H), 7.64 (d, J=8.2, 1H), 4.55 (s, 2H), 4.26-4.21 (m, 2H), 4.15 (dd, J=14.3, 7.1, 1H), 3.99-3.94 (m, 2H), 3.87-3.82 (m, 4H), 3.65 (t, J=5.8, 2H), 3.19-3.14 (m, 1H), 2.97 (s, 3H), 2.94-2.91 (m, 2H), 2.23-2.18 (m, 2H), 2.02-1.97 (m, 1H), 1.17 (t, J=7.1, 3H).

C. {5-[5-Methanesulfonyl-1-(3-oxo-propyl)-4,5,6,7-tetrahydro-1H-pyrazolo[4,3-c]pyridin-3-yl]-2-trifluoromethyl-phenylsulfanyl}-acetic acid ethyl ester. This compound was prepared using methods similar to those described in Example 1, Step E. MS (ESI): mass calcd. for C₂₁H₂₄F₃N₃O₅S₂, 519.11; m/z found, 520.1 [M+H]⁺.

D. [5-(5-Methanesulfonyl-1-[3-[4-(2-oxo-pyrrolidin-1-yl)-piperidin-1-yl]-propyl]-4,5,6,7-tetrahydro-1H-pyrazolo[4,3-c]pyridin-3-yl)-2-trifluoromethyl-phenylsulfanyl]-acetic acid ethyl ester. This compound was prepared using methods similar to those described in Example 1, Step F. MS (ESI): mass calcd. for C₃₀H₄₀F₃N₅O₅S₂, 671.24; m/z found, 672.1 [M+H]⁺. ¹H NMR: 8.07 (s, 1H), 7.76 (d, J=8.2, 1H), 7.60 (d, J=8.1, 1H), 4.72-4.33 (m, 4H), 4.56 (s, 2H), 4.30 (d, J=6.3, 3H), 4.19-4.10 (m, 1H), 3.92 (s, 2H), 3.75 (d, J=11.2, 2H), 3.63 (t, J=5.8, 2H), 3.41 (t, J=7.61 (2H), 3.27 (t, J=6.9, 2H), 3.23-3.14 (m, 2H), 2.98-2.93 (m, 2H), 2.95 (s, 3H), 2.54-2.46 (m, 2H), 2.23 (t, J=8.1, 2H), 2.20-2.09 (m, 2H), 1.99-1.91 (m, 2H), 1.86-1.78 (m, 2H).

E. [5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetic acid. This compound was prepared using methods similar to those described in Example 64. MS (ESI): mass calcd. for C₂₈H₃₆F₃N₅O₅S₂, 643.21; m/z found, 644.1 [M+H]⁺. ¹H NMR: 7.72 (s, 1H), 7.68 (d, J=8.2, 1H), 7.58 (d, J=8.0, 1H), 4.56 (s, 2H), 4.15 (t, J=6.6, 2H), 3.86-3.79 (m, 1H), 3.70 (s, 2H), 3.65 (t, J=5.8, 2H), 3.41 (t, J=7.0, 2H), 3.04 (s, 3H), 2.98-2.92 (m, 4H), 2.36 (dd, J=15.8, 7.8, 4H), 2.11-2.05 (m, 2H), 2.05-1.97 (m, 4H), 1.74-1.66 (m, 2H), 1.63-1.57 (m, 2H).

F. 1-{1-[3-(3-{3-[2-(3-Hydroxy-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one. This compound was prepared using methods similar to those described in Example 65, substituting PyBOP for EDC, HOAt for HOBt, DIPEA for TEA, and 3-pyrrolidinol for pyrrolidine. MS (ESI): mass calcd. for C₃₂H₄₃F₃N₆O₅S₂, 712.27; m/z found, 713.4 [M+H]⁺. ¹H NMR: 8.08 (s, 1H), 7.77 (d, J=8.2, 1H), 7.66 (d, J=8.1, 1H), 4.57 (s, 2H), 4.48-4.38 (m, 1H), 4.27 (t, J=6.2, 2H), 4.14-4.06 (m, 1H), 3.94 (s, 1H), 3.91 (s, 1H), 3.76-3.62 (m, 6H), 3.58-3.42 (m, 3H), 3.34 (s, 1H), 3.30-3.24 (m, 3H), 3.16-3.08 (m, 2H), 3.00 (s, 3H), 2.96-2.91 (m, 2H), 2.40-2.30 (m, 4H), 2.12-1.96 (m, 4H), 1.96-1.86 (m, 4H).

Examples 76-91 were prepared using methods similar to those described in Example 75, with the appropriate substituent changes.

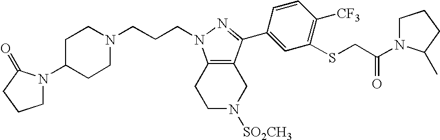

### Example 76

### 1-{1-[3-(5-Methanesulfonyl-3-{3-[2-(2-methyl-pyrrolidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₅F₃N₆O₄S₂, 710.29; m/z found, 711.4 [M+H]⁺. ¹H NMR: 7.97 (s, 1H), 7.68 (d, J=8.2, 1H), 7.57 (d, J=8.2, 1H), 4.48 (s, 2H), 4.20-4.14 (m, 2H), 4.04-3.94 (m, 2H), 3.80 (s, 1H), 3.60-3.52 (m, 5H), 3.46-3.28 (m, 2H), 3.20-3.14 (m, 4H), 3.06-2.98 (m, 2H), 3.90 (s, 3H), 2.86-2.82 (m, 2H), 2.30-2.20 (m, 4H), 1.98-1.86 (m, 4H), 1.88-1.78 (m, 5H), 1.64-1.48 (m, 1H), 1.10 (d, J=6.5, 1H), 1.01 (d, J=6.4, 2H).

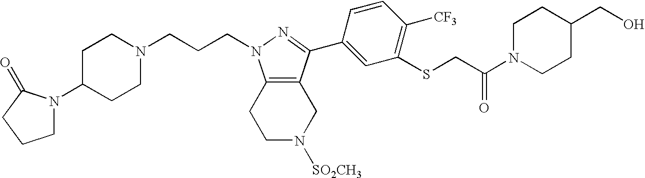

### Example 77

### 1-{1-[3-(3-{3-[2-(4-Hydroxymethyl-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₇F₃N₆O₅S₂, 740.30; m/z found, 741.4 [M+H]⁺. ¹H NMR: 8.10 (s, 1H), 7.77 (d, J=8.2, 1H), 7.66 (d, J=8.0, 1H), 4.58 (s, 2H), 4.54-4.48 (m, 2H), 4.32-4.24 (m, 2H), 4.14-3.94 (m, 4H), 3.74-3.62 (m, 3H), 3.38 (d, J=6.1, 2H), 3.34 (s, 1H), 3.33-3.29 (m, 4H), 3.30-3.25 (m, 2H), 3.20-3.08 (m, 2H), 3.00 (s, 3H), 2.97-2.92 (m, 2H), 2.69-2.62 (m, 1H), 2.40-2.31 (m, 3H), 2.05-1.98 (m, 2H), 1.97-1.89 (m, 3H), 1.85-1.68 (m, 3H), 1.27-1.15 (m, 1H), 1.12-1.00 (m, 1H).

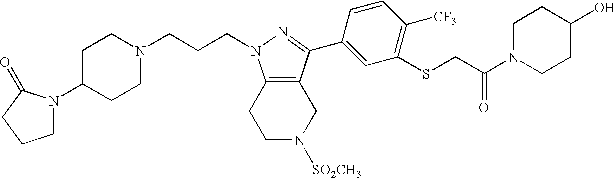

### Example 78

### 1-{1-[3-(3-{3-[2-(4-Hydroxy-piperidin-1-yl)-2-oxo-ethylsulfanyl]-4-trifluoromethyl-phenyl-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₅F₃N₆O₅S₂, 726.28; m/z found, 727.4 [M+H]⁺. ¹H NMR: 8.08 (s, 1H), 7.77 (d, J=8.2, 1H), 7.65 (d, J=8.1, 1H), 4.58 (s, 2H), 4.28 (t, J=6.2, 2H), 4.14-3.98 (m, 4H), 3.88-3.82 (m, 2H), 3.72-3.61 (m, 4H), 3.38-3.24 (m, 5H), 3.20-3.08 (m, 3H), 3.00 (s, 3H), 2.96-2.91 (m, 2H), 2.36 (t, J=8.1, 2H), 2.36-2.30 (m, 2H), 2.06-1.99 (m, 2H), 1.96-1.88 (m, 5H), 1.86-1.78 (m, 1H), 1.60-1.52 (m, 1H), 1.46-1.39 (m, 1H).

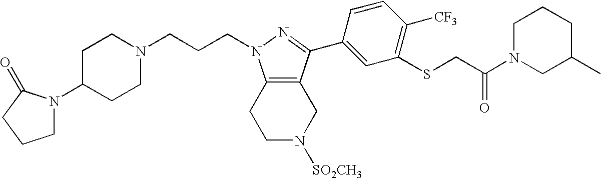

### Example 79

### 1-{1-[3-(5-Methanesulfonyl-3-{3-[2-(3-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₇F₃N₆O₄S₂, 724.31; m/z found, 725.4 [M+H]⁺. ¹H NMR: 8.10 (s, 1H), 7.78 (d, J=8.2, 1H), 7.66 (d, J=8.2, 1H), 4.58 (s, 2H), 4.38-4.22 (m, 1H), 4.28 (t, J=6.1, 2H), 4.13-3.92 (m, 4H), 3.89-3.79 (m, 1H), 3.67 (t, J=5.6, 3H), 3.64 (s, 1H), 3.30-3.25 (m, 4H), 3.13-3.09 (m, 2H), 3.00 (s, 3H), 2.95 (t, J=5.6, 2H), 2.78-2.60 (m, 1H), 2.42-2.31 (m, 5H), 2.06-1.98 (m, 3H), 1.96-1.91 (m, 5H), 1.84-1.79 (m, 1H), 1.78-1.66 (m, 1H), 1.63-1.32 (m, 2H), 1.22-1.14 (m, 1H).

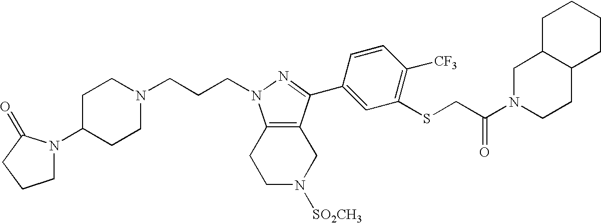

### Example 80

### 1-{1-[3-(5-Methanesulfonyl-3-{3-[2-(octahydro-isoquinolin-2-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₇H₅₁F₃N₆O₄S₂, 764.34; m/z found, 765.5 [M+H]⁺. ¹H NMR: 8.09 (s, 1H), 7.77 (d, J=8.2, 1H), 7.65 (d, J=8.3, 1H), 4.58 (s, 2H), 4.78 (t, J=6.2, 2H), 4.14-3.98 (m, 4H), 3.94-3.84 (m, 1H), 3.70-3.63 (m, 4H), 3.30-3.24 (m, 3H), 3.18-3.08 (m, 3H), 3.00 (s, 3H), 2.96-2.92 (m, 2H), 2.40-2.31 (m, 4H), 2.06-1.98 (m, 2H), 1.96-1.83 (m, 6H), 1.82-1.66 (m, 2H), 1.65-1.56 (m, 2H), 1.55-1.43 (m, 3H), 1.42-1.33 (m, 3H), 1.31-1.23 (m, 2H).

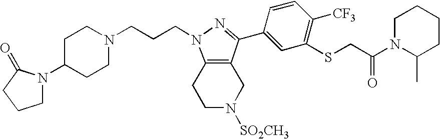

### Example 81

### 1-{1-[3-(5-Methanesulfonyl-3-{3-[2-(2-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₇F₃N₆O₄S₂, 724.31; m/z found, 725.4 [M+H]⁺. ¹H NMR: 8.11 (d, J=12.4, 1H), 7.78 (d, J=8.1, 1H), 7.65 (d, J=7.9, 1H), 4.58 (s, 2H), 4.36-4.26 (m, 1H), 4.28 (t, J=6.2, 2H), 4.11-4.05 (m, 1H), 4.04-3.92 (m, 2H), 3.78-3.72 (m, 1H), 3.67 (t, J=5.8, 3H), 3.64 (s, 1H), 3.34 (s, 3H), 3.30-3.26 (m, 2H), 3.26-3.22 (m, 3H), 3.00 (s, 3H), 2.96 (t, J=5.7, 2H), 2.38-2.30 (m, 4H), 2.04-1.96 (m, 2H), 1.94-1.88 (m, 3H), 1.76-1.66 (m, 2H), 1.64-1.54 (m, 3H), 1.33 (d, J=6.9, 2H), 1.12 (d, J=7.0, 2H).

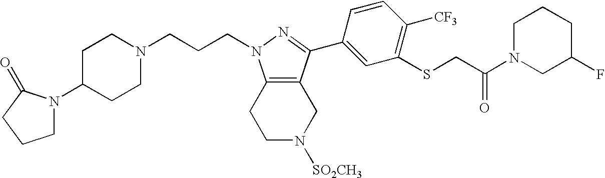

### Example 82

### 1-{1-[3-(3-{3-[2-(3-Fluoro-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₄F₄N₆O₄S₂, 728.28; m/z found, 729.4 [M+H]⁺. ¹H NMR: 8.07 (s, 0.6H), 8.01 (s, 0.4H), 7.80-7.75 (m, 1H), 7.68-7.62 (m, 1H), 4.84-4.62 (m, 1H), 4.57 (s, 2H), 4.32-4.26 (m, 2H), 4.19-3.91 (m, 5H), 3.80-3.73 (m, 1H), 3.70-3.62 (m, 4H), 3.44-3.36 (m, 2H), 3.30-3.20 (m, 2H), 3.16-3.08 (m, 3H), 2.99 (s, 3H), 2.97-2.91 (m, 2H), 2.42-2.31 (m, 4H), 2.05-1.95 (m, 3H), 1.96-1.84 (m, 5H), 1.82-1.70 (m, 1H), 1.66-1.48 (m, 1H).

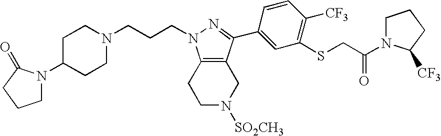

### Example 83

### (S)-1-{1-[3-(5-Methanesulfonyl-3-{3-[2-oxo-2-(2-trifluoromethyl-pyrrolidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₂F₆N₆O₄S₂, 764.26; m/z found, 765.5 [M+H]⁺. ¹H NMR: 8.05-7.98 (m, 1H), 7.72-7.66 (m, 1H), 7.59-7.54 (m, 1H), 4.49 (s, 2H), 4.18 (t, J=6.2, 2H), 4.04-3.94 (m, 1H), 3.63-3.52 (m, 4H), 3.20-3.14 (m, 3H), 3.10-2.99 (m, 2H), 2.91 (s, 3H), 2.85 (t, J=5.7, 2H), 2.30-2.22 (m, 4H), 1.96-1.88 (m, 2H), 1.86-1.78 (m, 4H), 1.78-1.73 (m, 1H), 1.66-1.58 (m, 3H), 1.54-1.44 (m, 2H), 1.34-1.16 (m, 4H).

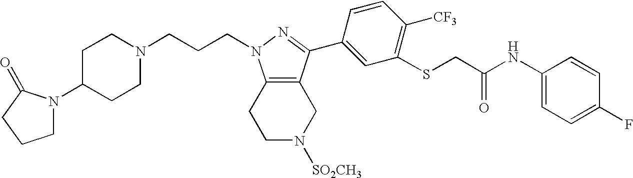

### Example 84

### N-(4-Fluoro-phenyl)-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetamide

MS (ESI): mass calcd. for C₃₄H₄₀F₄N₆O₄S₂, 736.25; m/z found, 738.0 [M+H]⁺. ¹H NMR (CDCl₃): 8.55 (s, 1H), 7.69 (d, J=8.2, 1H), 7.64 (s, 1H), 7.58 (d, J=8.2, 1H), 7.43-7.38 (m, 2H), 6.98 (t, J=8.7, 2H), 4.48 (s, 2H), 4.06 (t, J=6.8, 2H), 3.99-3.91 (m, 1H), 3.90 (s, 2H), 3.62 (t, J=5.8, 2H), 3.33 (t, J=7.0, 2H), 2.93 (s, 3H), 2.91-2.85 (m, 4H), 2.38 (t, J=8.1, 2H), 2.30 (t, J=6.9, 2H), 2.07-1.95 (m, 6H), 1.71-1.62 (m, 4H).

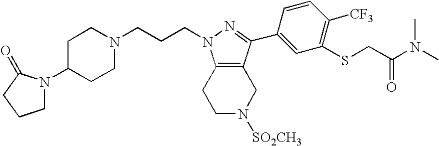

### Example 85

### 2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-N,N-dimethyl-acetamide

MS (ESI): mass calcd. for C₃₀H₄₁F₃N₆O₄S₂, 670.26; m/z found, 672.0 [M+H]⁺. ¹H NMR (CDCl₃): 7.96 (s, 1H), 7.70-7.62 (m, 2H), 4.63 (s, 2H), 4.08 (t, J=6.8, 2H), 4.00-3.91 (m, 1H), 3.79 (s, 2H), 3.65 (t, J=5.7, 2H), 3.34 (t, J=7.0, 2H), 3.08 (s, 3H), 2.98 (s, 3H), 2.95 (s, 3H), 2.93-2.86 (m, 4H), 2.39 (t, J=8.1, 2H), 2.32 (t, J=6.8, 2H), 2.10-1.94 (m, 6H), 1.71-1.68 (m, 4H).

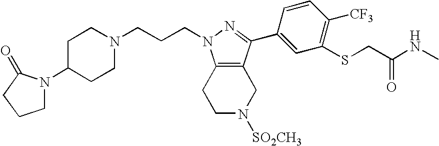

### Example 86

### 2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-N-methyl-acetamide

MS (ESI): mass calcd. for C₂₉H₃₉F₃N₆O₄S₂, 656.2; m/z found, 658.0 [M+H]⁺. ¹H NMR (CDCl₃): 7.72 (m, 2H), 7.28 (s, 1H), 6.89-6.82 (m, 1H), 4.45 (s, 2H), 4.08 (t, J=6.8, 2H), 3.99-3.91 (m, 1H), 3.72 (s, 2H), 3.64 (t, J=5.7, 2H), 3.34 (t, J=7.0, 2H), 3.07 (s, 3H), 2.89 (t, J=5.5, 4H), 2.84 (d, J=4.9, 3H), 2.38 (t, J=8.1, 2H), 2.32 (t, J=6.7, 2H), 2.08-1.95 (m, 6H), 1.68-1.61 (m, 4H).

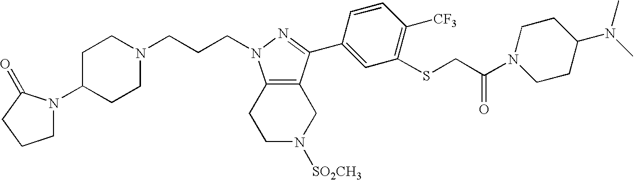

### Example 87

### 1-{1-[3-(3-{3-[2-(4-Dimethylamino-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₅H₅₀F₃N₇O₄S₂, 753.33; m/z found, 754.5 [M+H]⁺. ¹H NMR: 8.05 (s, 1H), 7.77 (d, J=8.2, 1H), 7.69 (d, J=8.0, 1H), 4.71-4.67 (m, 1H), 4.58 (s, 2H), 4.27 (t, J=6.2, 2H), 4.24-4.17 (m, 1H), 4.12-4.04 (m, 1H), 4.07 (s, 2H), 3.72-3.64 (m, 4H), 3.54-3.45 (m, 1H), 3.34 (s, 1H), 3.34-3.31 (m, 2H), 3.30-3.26 (m, 1H), 3.25-3.19 (m, 1H), 3.16-3.08 (m, 2H), 3.00 (s, 3H), 2.97-2.92 (m, 2H), 2.90-2.84 (m, 6H), 2.74-2.64 (m, 1H), 2.42-2.32 (m, 4H), 2.20-2.08 (m, 2H), 2.06-1.98 (m, 2H), 1.98-1.88 (m, 4H), 1.80-1.70 (m, 1H), 1.60-1.50 (m, 1H).

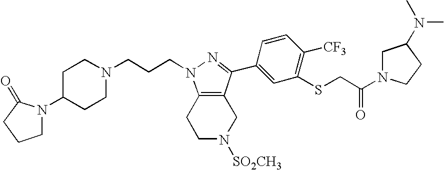

### Example 88

### 1-{1-[3-(3-{3-[2-(3-Dimethylamino-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₈F₃N₇O₄S₂, 739.32; m/z found, 740.4 [M+H]⁺. ¹H NMR: 7.96 (s, 0.4H), 7.93 (s, 0.6H), 7.67 (d, J=8.2, 1H), 7.64-7.58 (m, 1H), 4.48 (s, 2H), 4.17 (t, J=6.3, 2H), 4.12-4.07 (m, 1H), 4.05-3.95 (m, 1H), 3.94-3.86 (m, 3H), 3.86-3.76 (m, 1H), 3.75-3.60 (m, 2H), 3.60-3.52 (m, 4H), 3.52-3.45 (m, 1H), 3.42-3.35 (m, 1H), 3.20-3.17 (m, 2H), 3.09-2.98 (m, 2H), 2.92 (s, 2H), 2.90 (s, 1H), 2.87 (s, 3H), 2.86-2.81 (m, 2H), 2.83 (s, 3H), 2.49-2.33 (m, 1H), 2.30-2.21 (m, 2H), 2.27 (t, J=8.1, 2H), 2.21-2.01 (m, 1H), 1.97-1.89 (m, 2H), 1.89-1.76 (m, 4H).

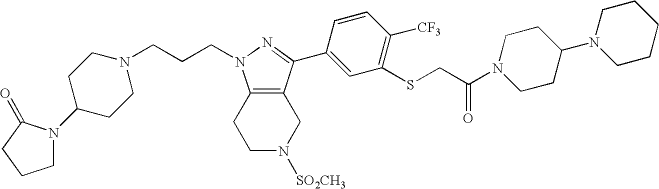

### Example 89

### 1-[1-(3-{3-[3-(2-[1,4′]Bipiperidinyl-1′-yl-2-oxo-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₈H₅₄F₃N₇O₄S₂, 793.36; m/z found, 794.5 [M+H]⁺.

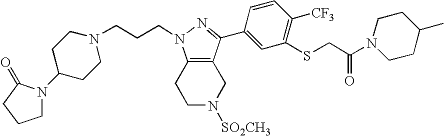

### Example 90

### 1-{1-[3-(5-Methanesulfonyl-3-{3-[2-(4-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₄H₄₇F₃N₆O₄S₂, 724.31; m/z found, 725.4 [M+H]⁺.

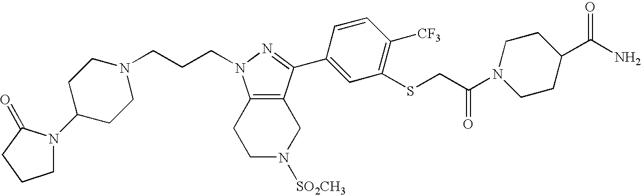

### Example 91

### 1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetyl}-piperidine-4-carboxylic acid amide

MS (ESI): mass calcd. for C₃₄H₄₆F₃N₇O₅S₂, 753.30; m/z found, 754.4 [M+H]⁺.

Examples 92 through 109 were prepared using methods similar to those described in Example 75, substituting HATU for PyBOP and the appropriate amine for 3-pyrrolidinol.

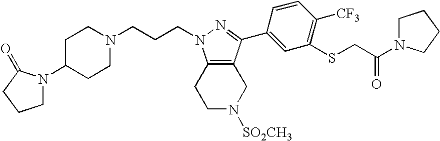

### Example 92

### 1-[1-(3-{5-Methanesulfonyl-3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one

MS (ESI): mass calcd. for C₃₂H₄₃F₃N₆O₄S₂, 696.27; m/z found, 697.4 [M+H]⁺. ¹H NMR: 8.11 (s, 1H), 7.78 (d, J=8.2, 1H), 7.67 (d, J=8.0, 1H), 4.57 (s, 2H), 4.32-4.27 (m, 2H), 4.13-4.05 (m, 1H), 3.91 (s, 2H), 3.72-3.64 (m, 4H), 3.58-3.54 (m, 2H), 3.45-3.40 (m, 2H), 3.29-3.24 (m, 3H), 3.17-3.08 (m, 2H), 3.00 (s, 3H), 2.98-2.93 (m, 2H), 2.42-2.31 (m, 4H), 2.05-2.01 (m, 2H), 2.01-1.95 (m, 3H), 1.95-1.90 (m, 3H), 1.90-1.84 (m, 3H).

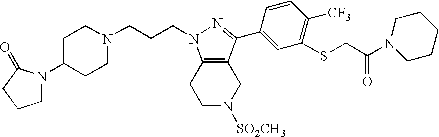

### Example 93

### 1-[1-(3-{5-Methanesulfonyl-3-[3-(2-oxo-2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one

MS (ESI): mass calcd. for C₃₃H₄₅F₃N₆O₄S₂, 710.29; m/z found, 711.5 [M+H]⁺. ¹H NMR: 8.10 (s, 1H), 7.78 (d, J=8.0, 1H), 7.66 (d, J=8.3, 1H), 4.58 (s, 2H), 4.32-4.26 (m, 2H), 4.12-4.04 (m, 1H), 3.99 (s, 2H), 3.72-3.66 (m, 3H), 3.56-3.50 (m, 5H), 3.30-3.22 (m, 3H), 3.14-3.08 (m, 2H), 3.00 (s, 3H), 2.96-2.91 (m, 2H), 2.40-2.32 (m, 4H), 2.04-1.98 (m, 2H), 1.96-1.88 (m, 4H), 1.70-1.60 (m, 5H), 1.54-1.48 (m, 2H).

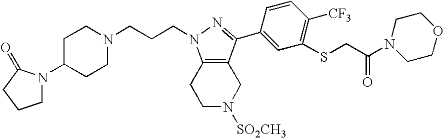

### Example 94

### 1-[1-(3-{5-Methanesulfonyl-3-[3-(2-morpholin-4-yl-2-oxo-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one

MS (ESI): mass calcd. for C₃₂H₄₃F₃N₆O₅S₂, 712.27; m/z found, 713.4 [M+H]⁺. ¹H NMR: 8.07 (s, 1H), 7.78 (d, J=8.2, 1H), 7.68 (d, J=8.1, 1H), 4.58 (s, 2H), 4.23 (t, J=6.3, 2H), 4.12-4.06 (m, 1H), 4.02 (s, 2H), 3.71-3.66 (m, 5H), 3.65-3.62 (m, 4H), 3.62-3.58 (m, 2H), 3.58-3.54 (m, 2H), 3.30-3.25 (m, 3H), 3.17-3.08 (m, 2H), 3.00 (s, 3H), 2.96-2.93 (m, 2H), 2.37 (t, J=8.1, 2H), 2.35-2.31 (m, 2H), 2.06-1.99 (m, 2H), 1.97-1.89 (m, 4H).

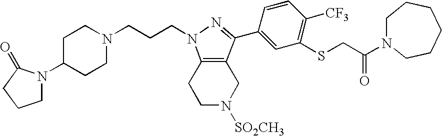

### Example 95

### 1-[1-(3-{3-[3-(2-Azepan-1-yl-2-oxo-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₄H₄₇F₃N₆O₄S₂, 724.31; m/z found, 725.5 [M+H]⁺. ¹H NMR: 8.10 (s, 1H), 7.78 (d, J=8.2, 1H), 7.67 (d, J=8.0, 1H), 4.58 (s, 2H), 4.28 (t, J=6.1, 2H), 4.12-4.04 (m, 1H), 3.99 (s, 2H), 3.70-3.65 (m, 3H), 3.64 (s, 1H), 3.59 (t, J=6.1, 2H), 3.51 (t, J=5.9, 2H), 3.30-3.22 (m, 4H), 3.14-3.08 (m, 2H), 3.00 (s, 3H), 2.96-2.92 (m, 2H), 2.40-2.32 (m, 4H), 2.04-1.98 (m, 2H), 1.96-1.88 (m, 4H), 1.82-1.77 (m, 2H), 1.70-1.64 (m, 2H), 1.62-1.52 (m, 4H).

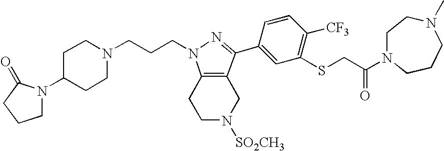

### Example 96

### 1-{1-[3-(5-Methanesulfonyl-3-{3-[2-(4-methyl-[1,4]diazepan-1-yl)-2-oxo-ethylsulfanyl]-4-trifluoromethyl-phenyl}-4,5,6,7-tetrahydro-pyrazolo[4,3c]pyridin-1-yl)-propyl]-piperidin-4-yl}-pyrrolidin-2-one

MS (ESI): mass calcd. for C₃₄H₄₈F₃N₇O₄S₂, 739.32; m/z found, 740.5 [M+H]⁺. ¹H NMR: 8.01 (s, 1H), 7.77 (d, J=8.3, 1H), 7.72 (d, J=8.8, 1H), 4.59 (s, 2H), 4.27 (t, J=6.3, 2H), 4.15-4.05 (s, 2H), 3.83-3.76 (m, 2H), 3.72-3.63 (m, 5H), 3.63-3.48 (m, 3H), 3.34 (s, 3H), 3.30-3.25 (m, 2H), 3.19 (s, 1H), 3.16-3.08 (m, 2H), 3.00 (s, 3H), 2.97-2.92 (m, 3H), 2.91 (s, 2H), 2.42-2.32 (m, 4H), 2.32-2.23 (m, 1H), 2.20-2.11 (m, 1H), 2.07-2.00 (m, 3H), 2.00-1.89 (m, 3H).

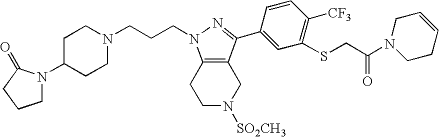

### Example 97

### 1-{1-[3-(3-{3-[2-(3,6-Dihydro-2H-pyridin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₃F₃N₆O₄S₂, 708.27; m/z found, 709.4 [M+H]⁺. ¹H NMR: 8.07 (m, 1H), 7.78 (d, J=8.1, 1H), 7.66 (d, J=8.2, 1H), 5.90-5.82 (m, 1H), 5.74-5.64 (m, 1H), 4.58 (s, 2H), 4.28 (t, J=6.2, 2H), 4.11-4.06 (m, 2H), 4.04 (s, 1H), 4.02-3.98 (m, 2H), 3.70-3.61 (m, 6H), 3.28-3.21 (m, 3H), 3.16-2.98 (m, 2H), 3.00 (s, 3H), 2.95 (t, J=5.4, 2H), 2.40-2.31 (m, 5H), 2.28-2.22 (m, 1H), 2.14-2.08 (m, 1H), 2.04-1.98 (m, 2H), 1.96-1.86 (m, 4H).

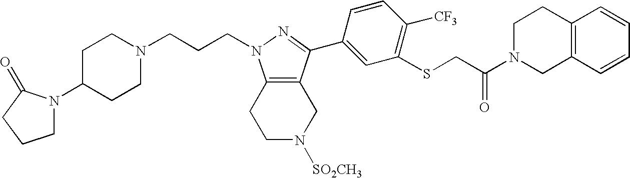

### Example 98

### 1-{1-[3-(3-{3-[2-(3,4-Dihydro-1H-isoquinolin-2-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₇H₄₅F₃N₆O₄S₂, 758.29; m/z found, 759.4 [M+H]⁺. ¹H NMR: 7.97 (d, J=8.0, 1H), 7.68 (d, J=8.1, 0.6H), 7.61 (d, J=8.1, 0.4H), 7.55 (d, J=8.0, 0.6H), 7.45 (d, J=8.0, 0.4H), 7.12-7.06 (m, 3H), 7.04-6.96 (m, 1H), 4.62 (s, 1H), 4.59 (s, 1H), 4.44 (s, 2H), 4.17 (t, J=6.2, 1H), 4.13 (t, J=6.1, 1H), 4.04-3.94 (m, 3H), 3.72 (t, J=5.9, 1H), 3.65 (t, J=5.9, 1H), 3.60-3.50 (m, 4H), 3.20-3.14 (m, 3H), 3.10 (t, J=7.0, 1H), 3.06-2.92 (m, 2H), 2.89 (d, J=3.1, 3H), 2.87-2.81 (m, 3H), 2.68 (t, J=5.9, 1H), 2.32-2.17 (m, 4H), 1.97-1.86 (m, 2H), 1.85-1.74 (m, 4H).

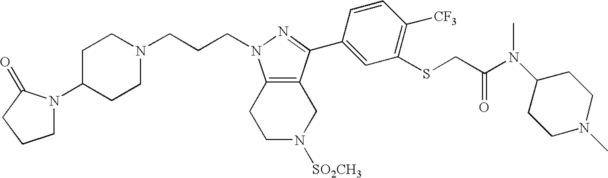

### Example 99

### 2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethylphenylsulfanyl]-N-methyl-N-(1-methyl-piperidin-4-yl)-acetamide

MS (ESI): mass calcd. for C₃₅H₅₀F₃N₇O₄S₂, 753.33; m/z found, 754.5 [M+H]⁺. ¹H NMR: 8.03 (s, 1H), 7.80-7.78 (m, 1H), 7.74-6.98 (m, 1H), 4.63-4.54 (m, 1H), 4.59 (s, 2H), 4.30-4.24 (m, 2H), 4.16-4.06 (m, 2H), 4.04 (s, 1H), 3.74-3.60 (m, 5H), 3.58-3.51 (m, 2H), 3.30-3.24 (m, 2H), 3.24-3.16 (m, 1H), 3.15-3.08 (m, 4H), 3.06-3.00 (m, 5H), 2.98-2.94 (m, 2H), 2.92-2.80 (m, 4H), 2.44-2.31 (m, 4H), 2.08-1.99 (m, 5H), 1.98-1.90 (m, 4H), 1.85-1.79 (m, 1H).

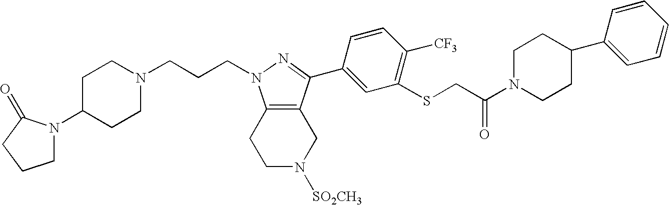

### Example 100

### 1-{1-[3-(5-Methanesulfonyl-3-{3-[2-oxo-2-(4-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₉H₄₉F₃N₆O₄S₂, 786.32; m/z found, 788.4 [M+H]⁺. ¹H NMR: 8.09 (s, 1H), 7.79 (d, J=8.2, 1H), 7.66 (d, J=7.9, 1H), 7.30-7.22 (m, 2H), 6.98 (d, J=7.9, 2H), 6.89 (t, J=7.3, 1H), 4.57 (s, 2H), 4.12-4.04 (m, 1H), 4.05 (s, 2H), 3.79-3.72 (m, 4H), 3.68-3.59 (m, 4H), 3.28-3.25 (m, 4H), 3.22-3.18 (m, 2H), 3.16-3.12 (m, 2H), 3.12-3.04 (m, 2H), 2.99 (s, 3H), 2.96-2.90 (m, 2H), 2.35 (t, J=8.1, 2H), 2.34-2.28 (m, 2H), 2.05-1.97 (m, 2H), 1.95-1.86 (m, 5H).

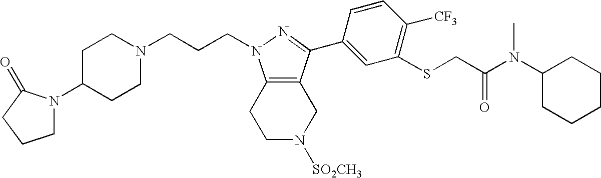

### Example 101

### N-Cyclohexyl-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethylphenylsulfanyl]-N-methyl-acetamide

MS (ESI): mass calcd. for C₃₅H₄₉F₃N₆O₄S₂, 738.32; m/z found, 739.5 [M+H]⁺. ¹H NMR: 8.11 (s, 0.5H), 8.05 (s, 0.5H), 7.79 (t, J=8.5, 1H), 7.69-7.64 (m, 1H), 4.57 (s, 2H), 4.32-4.22 (m, 1H), 4.27 (t, J=6.2, 2H), 4.13-4.04 (m, 1H), 3.99 (d, J=3.8, 2H), 3.72-3.62 (m, 4H), 3.30-3.24 (m, 4H), 3.17-3.07 (m, 2H), 3.00 (s, 3H), 2.98-2.92 (m, 4H), 2.80 (s, 1H), 2.39-2.31 (m, 4H), 2.05-1.97 (m, 2H), 1.97-1.87 (m, 4H), 1.86-1.74 (m, 2H), 1.74-1.50 (m, 4H), 1.48-1.28 (m, 3H), 1.19-1.09 (m, 1H).

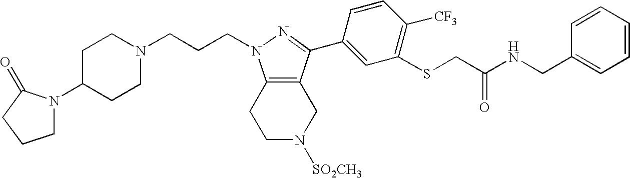

### Example 102

### N-Benzyl-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetamide

MS (ESI): mass calcd. for C₃₅H₄₃F₃N₆O₄S₂, 732.27; m/z found, 733.4 [M+H]⁺. ¹H NMR: 7.99 (s, 1H), 7.78 (d, J=8.2, 1H), 7.62 (d, J=7.9, 1H), 7.16-7.07 (m, 5H), 4.50 (s, 2H), 4.39-4.35 (m, 2H), 4.24 (t, J=6.1, 2H), 4.09-4.01 (m, 1H), 3.85 (s, 2H), 3.63 (t, J=5.8, 2H), 3.59 (d, J=11.8, 2H), 3.28-3.22 (m, 4H), 3.08-3.02 (m, 2H), 2.99 (s, 3H), 2.93 (t, J=5.5, 2H), 2.36 (d, J=8.1, 2H), 2.32-2.22 (m, 2H), 2.05-1.99 (m, 2H), 1.92-1.84 (m, 4H).

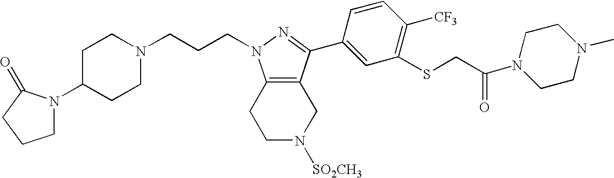

### Example 103

### 1-{1-[3-(5-Methanesulfonyl-3-{3-[2-(4-methyl-piperazin-1-yl)-2-oxo-ethylsulfanyl]-4-trifluoromethyl-phenyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₆F₃N₇O₄S₂, 725.30; m/z found, 726.5 [M+H]⁺. ¹H NMR: 8.02 (s, 1H), 7.77 (d, J=8.2, 1H), 7.71 (d, J=8.1, 1H), 4.58 (s, 2H), 4.27 (t, J=6.3, 2H), 4.16-4.05 (m, 2H), 4.12 (s, 2H), 3.72-3.62 (m, 2H), 3.67 (t, J=5.7, 2H), 3.34-3.24 (m, 10H), 3.18-3.06 (m, 3H), 3.00 (s, 3H), 2.98-2.92 (m, 2H), 2.93 (s, 3H), 2.44-2.31 (m, 4H), 2.08-1.86 (m, 6H).

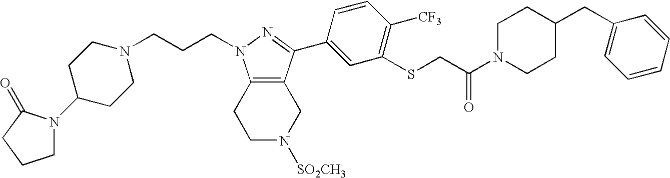

### Example 104

### 1-{1-[3-(3-{3-[2-(4-Benzyl-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₄₀H₅₁F₃N₆O₄S₂, 800.34; m/z found, 801.5 [M+H]⁺.

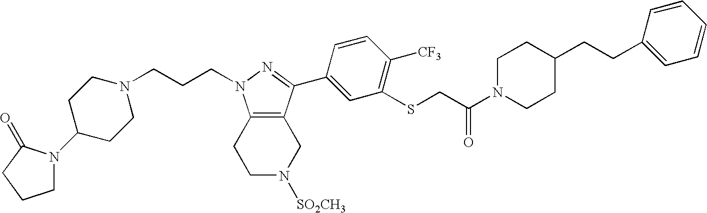

### Example 105

### 1-{1-[3-(5-Methanesulfonyl-3-{3-[2-oxo-2-(4-phenethyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₄₁H₅₃F₃N₆O₄S₂, 814.35; m/z found, 816.5, 817.5 [M+H]⁺.

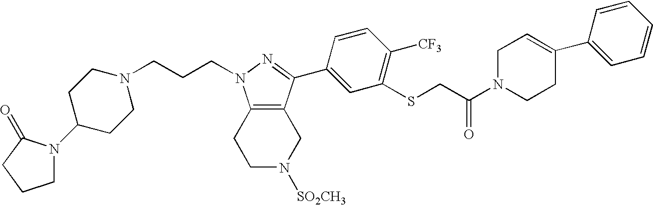

### Example 106

### 1-{1-[3-(5-Methanesulfonyl-3-{3-[2-oxo-2-(4-phenyl-3,6-dihydro-2H-pyridin-1-yl)-ethylsufanyl]-4-trifluoromethyl-phenyl}-4,5,6,7-tetrahydro-pyrazolo[4,3-c]pyridin-1-yl)-propyl]-piperidin-4-yl}-pyrrolidin-2-one

MS (ESI): mass calcd. for C₃₉H₄₇F₃N₆O₄S₂, 784.31; m/z found, 785.5 [M+H]⁺.

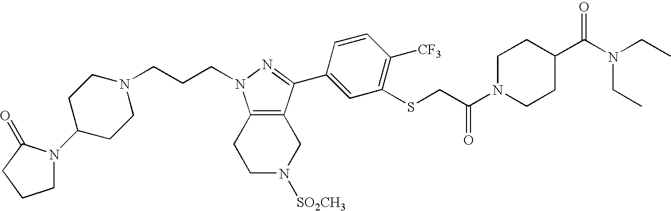

### Example 107

### 1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl phenylsulfanyl]-acetyl}-piperidine-4-carboxylic acid diethylamide

MS (ESI): mass calcd. for C₃₈H₅₄F₃N₇O₅S₂, 809.36; m/z found, 810.5 [M+H]⁺.

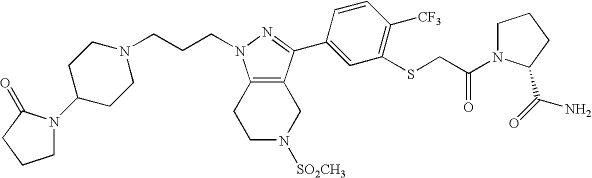

### Example 108

### (R)-1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetyl}-pyrrolidine-2-carboxylic acid amide

MS (ESI): mass calcd. for C₃₃H₄₄F₃N₇O₅S₂, 739.28; m/z found, 740.2 [M+H]⁺.

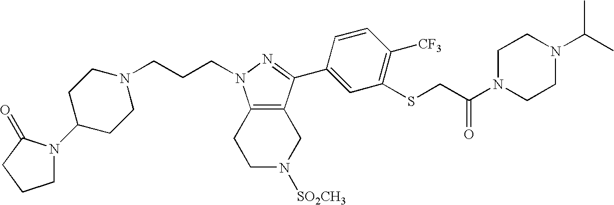

### Example 109

### 1-{1-[3-(3-{3-[2-(4-Isopropyl-piperazin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₅H₅₀F₃N₇O₄S₂, 753.33; m/z found, 754.5 [M+H]⁺.

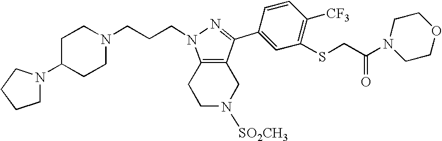

### Example 110

### 2-(5-{5-Methanesulfonyl-1-[3-(4-pyrrolidin-1-yl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone

A. {5-[1-(2-[1,3]Dioxolan-2-yl-ethyl)-5-methanesulfonyl-4,5,6,7-tetrahydro-1H-pyrazolo[4,3-c]pyridin-3-yl]-2-trifluoromethyl-phenylsulfanyl]-acetic acid. This compound was prepared using methods similar to those described in Example 64. MS (ESI): mass calcd. for C₂₁H₂₄F₃N₃O₆S₂, 535.11; m/z found, 536.1 [M+H]⁺. ¹H NMR: 7.63 (s, 1H), 7.58 (d, J=8.2, 1H), 7.48 (d, J=8.2, 1H), 4.46 (s, 2H), 4.13 (t, J=7.0, 2H), 3.90-3.86 (m, 3H), 3.77-3.73 (m, 3H), 3.61 (s, 1H), 3.55 (t, J=5.8, 2H), 2.92 (s, 3H), 2.82 (t, J=5.7, 2H), 2.13-2.08 (m, 2H).

B. 2-{5-[1-(2-[1,3]Dioxolan-2-yl-ethyl)-5-methanesulfonyl-4,5,6,7-tetrahydro-1H-pyrazolo[4,3-c]pyridin-3-yl]-2-trifluoromethyl-phenylsulfanyl}-1-morpholin-4-yl-ethanone. This compound was prepared using methods similar to those described in Example 65, substituting HATU for EDC, HOAt for HOBt, DIPEA for TEA, and morpholine for pyrrolidine. MS (ESI): mass calcd. for C₂₅H₃₁F₃N₄O₆S₂, 604.16; m/z found, 605.1 [M+H]⁺.

C. 2-(5-{5-Methanesulfonyl-1-[3-(4-pyrrolidin-1-yl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone. This compound was prepared using methods similar to those described in Example 1, Steps E and F, with the appropriate substituent changes. MS (ESI): mass calcd. for C₃₂H₄₅F₃N₆O₄S₂, 698.29; m/z found, 699.2 [M+H]⁺. ¹H NMR: 7.95 (s, 1H), 7.75 (d, J=8.3, 1H), 7.65 (d, J=8.3, 1H), 4.58 (s, 2H), 4.15 (t, J=6.7, 2H), 4.00 (s, 2H), 3.69-3.60 (m, 6H), 3.60-3.54 (m, 3H), 2.98-2.89 (m, 5H), 2.97 (s, 3H), 2.64-2.58 (m, 4H), 2.33-2.27 (m, 2H), 2.11-2.03 (m, 3H), 2.00-1.90 (m, 4H), 1.83-1.77 (m, 4H), 1.57-1.46 (m, 2H).

Examples 111-113 were prepared using methods similar to those described in Example 110, with the appropriate substituent changes.

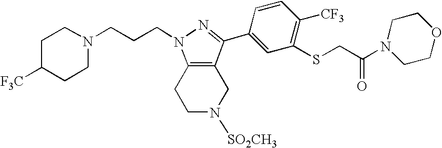

### Example 111

### 2-(5-{5-Methanesulfonyl-1-[3-(4-trifluoromethyl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone

MS (ESI): mass calcd. for C₂₉H₃₇F₆N₅O₄S₂, 697.22; m/z found, 698.2 [M+H]⁺. ¹H NMR: 7.94 (d, J=8.8, 1H), 7.76-7.70 (m, 1H), 7.65 (d, J=8.0, 1H), 4.57 (s, 2H), 4.15 (t, J=6.7, 2H), 4.00 (d, J=5.0, 2H), 3.68-3.60 (m, 5H), 3.60-3.52 (m, 3H), 3.12 (s, 1H), 3.02-2.90 (m, 5H), 2.97 (s, 3H), 2.36-2.31 (m, 2H), 2.18-2.00 (m, 3H), 1.99-1.90 (m, 2H), 1.86-1.80 (m, 2H), 1.61-1.50 (m, 2H).

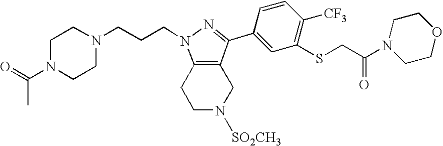

### Example 112

### 2-(5-{1-[3-(4-Acetyl-piperazin-1-yl)-propyl]-5-methanesulfonyl-4,5,6,7-tetrahydro-1H-pyrazolo[4,3-c]pyridin-3-yl}-2-trifluoromethyl-phenylsulfanyl)-1-morpholin-4-yl-ethanone

MS (ESI): mass calcd. for C₂₉H₃₉F₃N₆O₅S₂, 672.24; m/z found, 673.2 [M+H]⁺. ¹H NMR: 7.96-7.91 (m, 1H), 7.67-7.62 (m, 1H), 7.59-7.53 (m, 1H), 4.48 (s, 2H), 4.17-4.12 (m, 2H), 3.83 (d, J=9.2, 2H), 3.70 (br s, 3H), 3.60-3.56 (m, 3H), 3.56-3.51 (m, 2H), 3.48-3.42 (m, 3H), 3.33-3.26 (m, 1H), 3.18-3.09 (m, 5H), 3.03-2.98 (m, 1H), 2.88 (s, 3H), 2.85-2.80 (m, 3H), 2.32-2.24 (m, 2H), 2.08-1.96 (m, 1H), 2.00 (s, 3H).

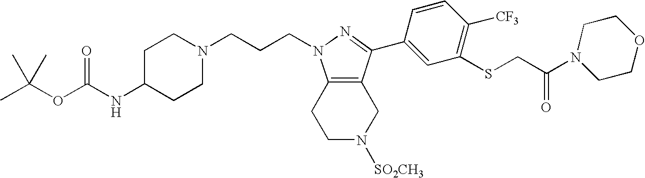

### Example 113

### [1-(3-{5-Methanesulfonyl-3-[3-(2-morpholin-4-yl-2-oxo-ethylsulfanyl)-4-trifluoromethyl-phenyl]-4,5,6,7-tetrahydro-pyrazolo[4,3-c]pyridin-1-yl}-propyl)-piperidin-4-yl]-carbamic acid tert-butyl ester

MS (ESI): mass calcd. for C₃₃H₄₇F₃N₆O₆S₂, 744.30; m/z found, 745.2 [M+H]⁺. ¹H NMR: 7.95 (s, 1H), 7.75 (d, J=8.2, 1H), 7.65 (d, J=8.2, 1H), 4.57 (s, 2H), 4.15 (t, J=6.6, 2H), 4.02-3.96 (m, 1H), 4.00 (s, 1H), 3.69-3.60 (m, 6H), 3.60-3.53 (m, 4H), 3.27-3.23 (m, 1H), 2.97 (s, 3H), 2.96-2.90 (m, 2H), 2.87-2.80 (m, 2H), 2.34 (t, J=7.3, 2H), 2.10-2.00 (m, 4H), 1.84-1.78 (m, 2H), 1.46-1.36 (m, 2H), 1.41 (s, 9H).

Examples 114 through 126 were prepared using methods similar to those described in Example 65, substituting HATU for EDC, HOAt for HOBt, DIPEA for TEA, and the appropriate amine for pyrrolidine.

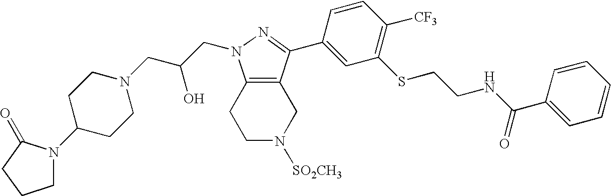

### Example 114

### N-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide

MS (ESI): mass calcd. for C₃₅H₄₃F₃N₆O₅S₂, 748.3; m/z found, 749.4 [M+H]⁺. ¹H NMR: 8.00 (s, 1H), 7.70 (t, J=8.0, 3H), 7.49 (t, J=6.7, 2H), 7.40 (t, J=7.7, 2H), 4.52 (br s, 2H), 4.49 (br s, 1H), 4.23-4.14 (m, 2H), 4.09-4.03 (m, 1H), 3.70-3.58 (m, 6H), 3.35-3.32 (m, 5H), 3.26-3.16 (m, 2H), 3.11-3.06 (m, 1H), 2.97-2.94 (m, 2H), 2.91 (s, 3H), 2.35 (t, J=8.0, 2H), 2.09-1.91 (m, 4H), 1.86-1.83 (m, 2H).

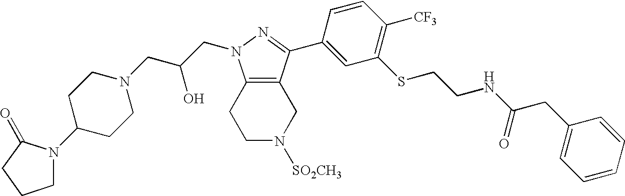

### Example 115

### N-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-2-phenyl-acetamide

MS (ESI): mass calcd. for C₃₆H₄₅F₃N₆O₅S₂, 762.3; m/z found, 763.4 [M+H]⁺. ¹H NMR: 7.93 (s, 1H), 7.74 (d, J=8.2, 1H), 7.56 (d, J=8.0, 1H), 7.28-7.25 (m, 2H), 7.21 (d, J=7.5, 3H), 4.53 (br s, 2H), 4.44 (br s, 1H), 4.23-4.14 (m, 2H), 4.09-4.02 (m, 1H), 3.64-3.58 (m, 4H), 3.47-3.45 (m, 4H), 3.35-3.31 (m, 2H), 3.26-3.23 (m, 1H), 3.22-3.13 (m, 4H), 3.05-3.00 (m, 1H), 2.97-2.95 (m, 2H), 2.91 (s, 3H), 2.36 (t, J=8.0, 2H), 2.04-1.99 (m, 3H), 1.94-1.92 (m, 1H), 1.86-1.83 (m, 2H).

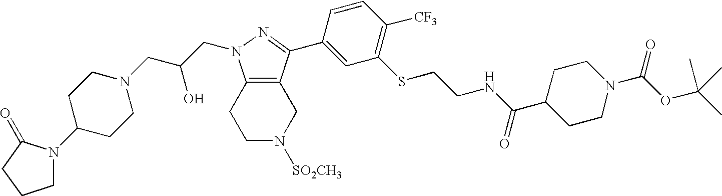

### Example 116

### 4-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-piperidine-1-carboxylic acid tert-butyl ester

MS (ESI): mass calcd. for C₃₉H₅₆F₃N₇O₇S₂, 855.4; m/z found, 856.5 [M+H]⁺. ¹H NMR: 7.88 (s, 1H), 7.72 (d, J=8.2, 1H), 7.56 (d, J=8.0, 1H), 4.54 (br s, 2H), 4.23-4.17 (m, 2H), 4.06-4.03 (m, 3H), 3.87-3.80 (m, 1H), 3.67-3.58 (m, 2H), 3.44-3.39 (m, 4H), 3.33 (m, 3H), 3.20 (t, J=6.7, 2H), 3.05-2.99 (m, 3H), 2.94 (s, 3H), 2.73 (br s, 2H), 2.53-2.45 (m, 2H), 2.35 (t, J=8.0, 2H), 2.21-2.16 (m, 2H), 2.02-1.96 (m, 2H), 1.80-1.73 (m, 2H), 1.69-1.67 (m, 2H), 1.61-1.58 (m, 2H), 1.53-1.45 (m, 2H), 1.43 (s, 9H).

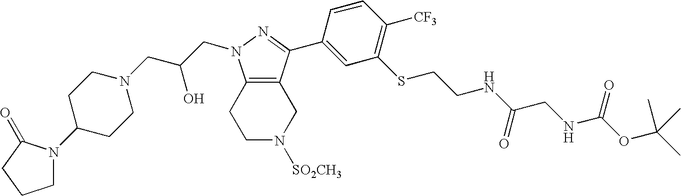

### Example 117

### ({2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-methyl)-carbamic acid tert-butyl ester

MS (ESI): mass calcd. for C₃₅H₅₀F₃N₇O₇S₂, 801.3; m/z found, 802.5 [M+H]⁺. ¹H NMR: 7.90 (s, 1H), 7.73 (d, J=8.2, 1H), 7.57 (d, J=8.2, 1H), 4.55 (br s, 2H), 4.24-4.19 (m, 2H), 4.08-4.04 (m, 1H), 3.88-3.81 (m, 1H), 3.67-3.59 (m, 4H), 3.46-3.39 (m, 4H), 3.33 (s, 1H), 3.19 (t, J=6.9, 2H), 3.06-2.96 (br m, 4H), 2.95 (s, 3H), 2.51-2.49 (m, 2H), 2.35 (t, J=8.1, 2H), 2.23-2.17 (m, 2H), 2.02-1.96 (m, 2H), 1.81-1.73 (m, 2H), 1.62-1.59 (m, 2H), 1.42 (s, 9H).

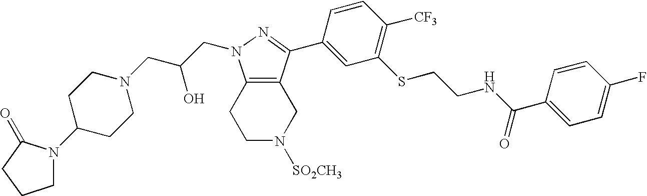

### Example 118

### 4-Fluoro-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide

MS (ESI): mass calcd. for C₃₅H₄₂F₄N₆O₅S₂, 766.26; m/z found, 767.4 [M+H]⁺.

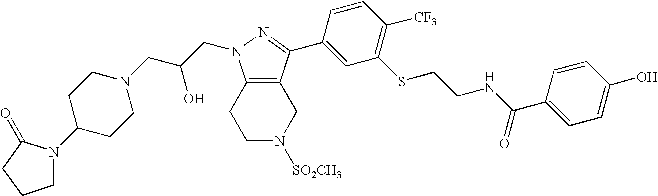

### Example 119

### 4-Hydroxy-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide

MS (ESI): insufficient ionization.

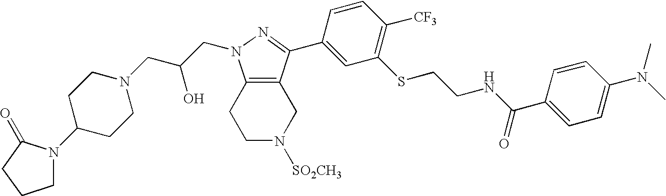

### Example 120

### 4-Dimethylamino-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide

MS (ESI): mass calcd. for C₃₇H₄₈F₃N₇O₅S₂, 791.31; m/z found, 792.5 [M+H]⁺.

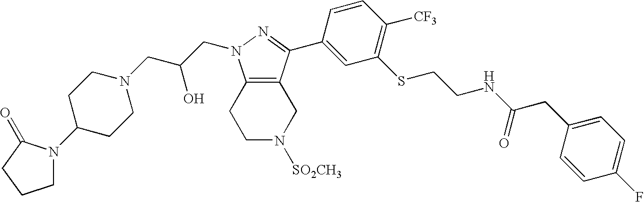

### Example 121

### 2-(4-Fluoro-phenyl)-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-acetamide

MS (ESI): mass calcd. for C₃₆H₄₄F₄N₆O₅S₂, 780.28; m/z found, 781.4 [M+H]⁺.

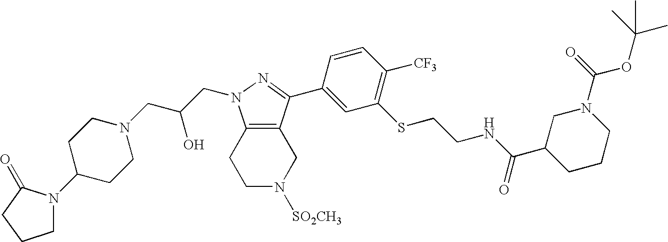

### Example 122

### 3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-piperidine-1-carboxylic acid tert-butyl ester

MS (ESI): mass calcd. for C₃₉H₅₆F₃N₇O₇S₂, 855.36; m/z found, 856.5 [M+H]⁺.

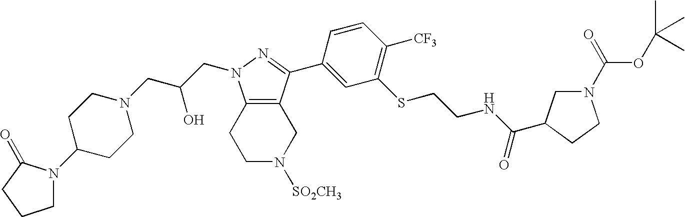

### Example 123

### 3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-pyrrolidine-1-carboxylic acid tert-butyl ester

MS (ESI): mass calcd. for C₃₈H₅₄F₃N₇O₇S₂, 841.35; m/z found, 842.5 [M+H]⁺.

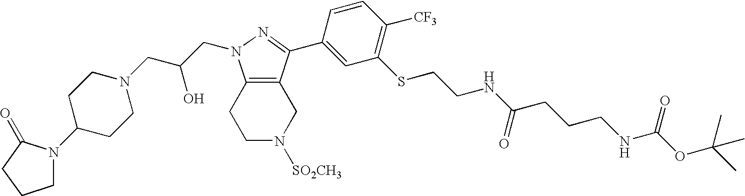

### Example 124

### (3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-propyl)-carbamic acid tert-butyl ester

MS (ESI): mass calcd. for C₃₇H₅₄F₃N₇O₇S₂, 829.35; m/z found, 830.5 [M+H]⁺.

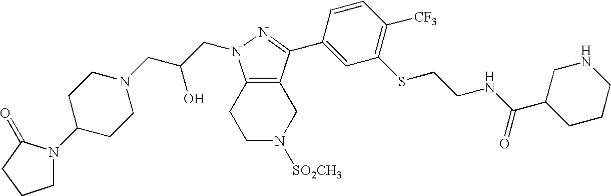

### Example 125

### Piperidine-3-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide

MS (ESI): mass calcd. for C₃₄H₄₈F₃N₇O₅S₂, 755.31; m/z found, 756.5 [M+H]⁺.

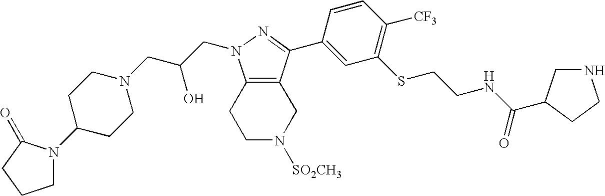

### Example 126

### Pyrrolidine-3-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide

MS (ESI): insufficient ionization.

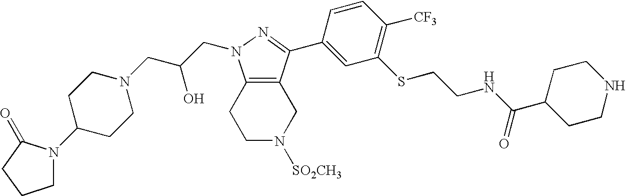

### Example 127

### Piperidine-4-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide

This compound was prepared using methods similar to those described in Example 64, with the appropriate substituent changes. MS (ESI): mass calcd. for C₃₄H₄₈F₃N₇O₅S₂, 755.3; m/z found, 756.5 [M+H]⁺. ¹H NMR: 7.89 (s, 1H), 7.74 (d, J=8.2, 1H), 7.59 (d, J=8.2, 1H), 4.56 (br s, 2H), 4.48-4.40 (m, 1H), 4.27-4.15 (m, 2H), 4.11-4.03 (m, 1H), 3.69-3.62 (m, 2H), 3.61-3.56 (m, 3H), 3.13-3.08 (m, 1H), 3.04-2.99 (m, 5H), 2.97 (s, 3H), 2.53-2.46 (m, 1H), 2.37 (t, J=8.1, 2H), 2.08-1.93 (m, 7H), 1.88-1.78 (m, 4H).

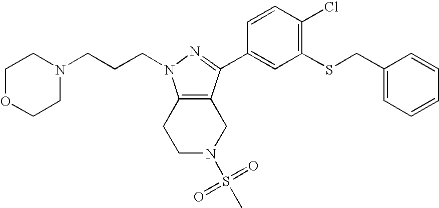

### Example 128

### 3-(3-Benzylsulfanyl-4-chloro-phenyl)-5-methanesulfonyl-1-(3-morpholin-4-yl-propyl)-4,5,6,7-tetrahydro-1H-pyrazolo[4,3-c]pyridine

A. 3-(4-Chloro-3-iodo-phenyl)-5-methanesulfonyl-4,5,6,7-tetrahydro-1H-pyrazolo[4,3-c]pyridine. This compound was prepared using the methods outlined in Example 1, Steps A and B, substituting 4-chloro-3-iodobenzoyl chloride for 4-trifluoromethyl-3-nitrobenzoyl chloride. MS (ESI): mass calcd. for C₁₃H₁₃CIIN₃O₂S, 437.7; m/z found, 438.1 [M+H]⁺. ¹H NMR (DMSO-d₆): 8.05 (d, J=1.9, 1H), 7.51 (d, J=8.3, 1H), 7.43 (dd, J=8.4, 1.9 2H), 4.30 (s, 2H), 3.36 (t, J=5.8, 2H), 3.30 (br s, 1H), 2.86 (s, 3H), 2.69 (t, J=5.6, 2H).

B. 3-(4-Chloro-3-iodo-phenyl)-5-methanesulfonyl-1-(3-morpholin-4-yl-propyl)-4,5,6,7-tetrahydro-1H-pyrazolo[4,3-c]pyridine. This compound was prepared using the methods outlined in Example 1, substituting 3-(4-chloro-3-iodo-phenyl)-5-methanesulfonyl-4,5,6,7-tetrahydro-1H-pyrazolo[4,3-c]pyridine for 2-[5-(5-methanesulfonyl-4,5,6,7-tetrahydro-1H-pyrazolo[4,3-c]pyridin-3-yl)-2-trifluoromethyl-phenylsulfanyl]-ethanol and morpholine for 4-(N-2-pyrrolidinone)piperidine. MS (ESI): mass calcd. for C₂₀H₂₆CIIN₄O₃S, 564.9; m/z found, 566.2 [M+H]⁺. ¹H NMR (CDCl₃): 8.15 (s, 1H), 7.46-7.45 (m, 2H), 4.49 (s, 2H), 4.09 (t, J=6.8, 2H), 3.70 (t, J=4.6, 4H), 3.64 (t, J=5.8, 2H), 2.90 (s, 3H), 2.89 (t, J=5.8, 2H), 2.44-2.39 (bm, 4H), 2.32 (t, J=6.8, 2H), 2.11-2.03 (m, 2H).

C. 3-(3-Benzylsulfanyl-4-chloro-phenyl)-5-methanesulfonyl-1-(3-morpholin-4-yl-propyl)-4,5,6,7-tetrahydro-1H-pyrazolo[4,3-c]pyridine. A solution of the above iodoarene (100 mg, 0.177 mmol), tris(dibenzylideneacetone)dipalladium (8.1 mg, 8.9×10⁻³ mmol), diphenylphosphinoferrocene (19.4 mg, 0.035 mmol) and TEA (50 μL, 0.354 mmol) in degassed DMF was stirred at rt under N₂ for 15 min. Benzylmercaptan (23 μL, 0.177 mmol) was added drop-wise and the reaction mixture was heated at 70° C. for 5 h. After 1 N NaOH (5 mL) was added, the two layers were separated, and the aqueous layer was extracted with CH₂Cl₂ (3×, 5 mL). The combined organic extracts were dried (Na₂SO₄) and filtered to give a yellow oil. Purification (SiO₂; 0-3%1:9 satd. NH₃ in MeOH/MeOH in CH₂Cl₂) afforded the desired product as a clear oil (94 mg, 95%). MS (ESI): mass calcd. for C₂₇H₃₃ClN₄O₃S₂, 561.2; m/z found, 562.5 [M+H]⁺. ¹H NMR (CDCl₃): 7.56 (d, J=2.0, 1H), 7.38 (d, J=8.3, 1H), 7.32-7.22 (m, 6H), 4.39 (s, 2H), 4.21 (s, 2H), 4.08 (t, J=6.8, 2H), 3.68 (t, J=4.6, 4H), 3.62 (t, J=5.8, 2H), 2.87-2.84 (m, 2H), 2.86 (s, 3H), 2.41-2.38 (m, 4H), 2.31 (t, J=6.8, 2H), 2.09-2.02 (m, 2H).

Examples 129 through 139 were prepared using methods similar to those described in Example 128, with the appropriate substituent changes.

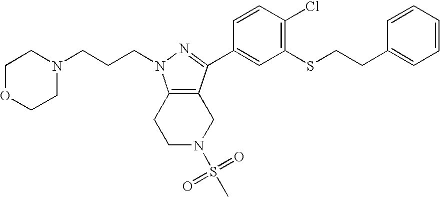

### Example 129

### 3-(4-Chloro-3-phenethylsulfanyl-phenyl)-5-methanesulfonyl-1-(3-morpholin-4-yl-propyl)-4,5,6,7-tetrahydro-1H-pyrazolo[4,3-c]pyridine

MS (ESI): mass calcd. for C₂₈H₃₅ClN₄O₃S₂, 575.2; m/z found, 576.3 [M+H]⁺. ¹H NMR (CDCl₃): 7.66 (d, J=2.0, 1H), 7.40 (d, J=8.2, 1H), 7.34-7.21 (m, 6H), 4.49 (s, 2H), 4.10 (t, J=6.8, 2H), 3.68 (t, J=4.6, 4H), 3.64 (t, J=5.8, 2H), 3.26-3.22 (m, 2H), 3.03-2.99 (m, 2H), 2.89 (t, J=5.7, 2H), 2.84 (s, 3H), 2.41-2.38 (br m, 4H), 2.32 (t, J=6.8, 2H), 2.10-2.05 (m, 2H).

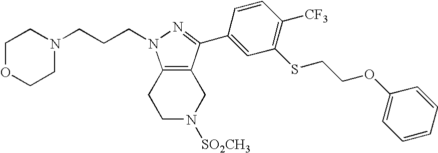

### Example 130

### 5-Methanesulfonyl-1-(3-morpholin-4-yl-propyl)-3-[3-(2-phenoxy-ethylsulfanyl)-4-trifluoromethyl-phenyl]-4,5,6,7-tetrahydro-1H-pyrazolo[4,3-c]pyridine

MS (ESI): mass calcd. for C₂₉H₃₅F₃N₄O₄S₂, 624.21; m/z found, 625.5 [M+H]⁺. ¹H NMR (CDCl₃): 7.92 (s, 1H), 7.71 (d, J=8.2, 1H), 7.43 (d, J=8.1, 1H), 7.28-7.23 (m, 2H), 6.95 (t, J=7.3, 1H), 6.86 (d, J=8.5, 2H), 4.50 (s, 2H), 4.21 (t, J=7.0, 2H), 4.16 (t, J=6.5, 2H), 3.97-3.87 (m, 4H), 3.64 (t, J=5.7, 2H), 3.49 (s, 1H), 3.48-3.43 (m, 1H), 3.41 (t, J=6.9, 2H), 3.12-3.05 (m, 2H), 2.87-2.82 (m, 2H), 2.85 (s, 3H), 2.81-2.72 (m, 2H), 2.41-2.33 (m, 2H).

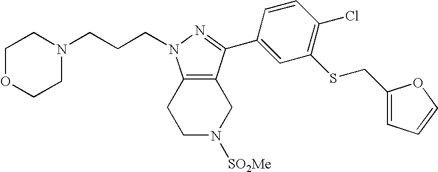

### Example 131

### 3-[4-Chloro-3-(furan-2-ylmethylsulfanyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₅H₃₁ClN₄O₄S₂, 550.15; m/z found, 551.5 [M+H]⁺. ¹H NMR (CDCl₃): 7.61 (d, J=1.9, 1H), 7.43-7.34 (m, 3H), 6.29-6.26 (m, 1H), 6.20 (d, J=3.2, 1H), 4.45 (s, 2H), 4.21 (s, 2H), 4.09 (t, J=6.8, 2H), 3.70 (t, J=4.5, 4H), 3.65 (t, J=5.8, 2H), 2.91-2.86 (m, 2H), 2.88 (s, 3H), 2.45-2.38 (m, 4H), 2.33 (t, J=6.7, 2H), 2.11-2.03 (m, 2H).

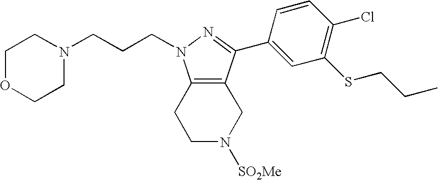

### Example 132

### 3-(4-Chloro-3-propylsulfanyl-phenyl)-5-methanesulfonyl-1-(3-morpholin-4-yl-propyl)-4,5,6,7-tetrahydro-1H-pyrazolo[4,3-c]pyridine

MS (ESI): mass calcd. for C₂₃H₃₃ClN₄O₃S₂, 512.17; m/z found, 513.5 [M+H]⁺. ¹H NMR: 7.59 (d, J=2.0, 1H), 7.43 (d, J=8.3, 1H), 7.34 (dd, J=8.5, 2.0, 1H), 4.49 (s, 2H), 4.16 (t, J=6.7, 2H), 3.69-3.62 (m, 6H), 3.01 (t, J=7.3, 2H), 2.99 (s, 3H), 2.97-2.93 (m, 2H), 2.42 (br s, 4H), 2.34 (t, J=7.1, 2H), 2.10-2.03 (m, 2H), 1.79-1.70 (m, 2H), 1.09 (t, J=7.4, 3H).

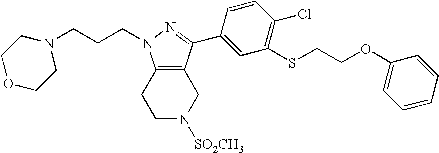

### Example 133

### 3-[4-Chloro-3-(2-phenoxy-ethylsulfanyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₈H₃₅ClN₄O₄S₂, 590.18; m/z found, 591.5 [M+H]⁺.

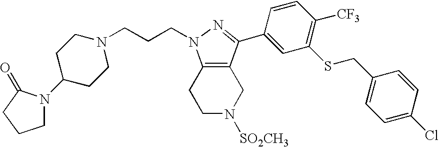

### Example 134

### 1-[1-(3-{3-[3-(4-Chloro-benzylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₃H₃₉ClF₃N₅O₃S₂, 709.21; m/z found, 710.6 [M+H]⁺.

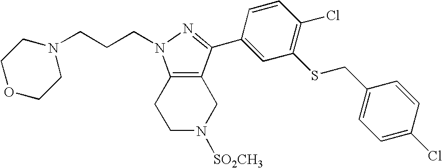

### Example 135

### 3-[4-Chloro-3-(4-chloro-benzylsulfanyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₇H₃₂Cl₂N₄O₃S₂, 594.13; m/z found, 595.5 [M+H]⁺.

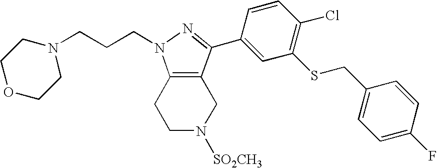

### Example 136

### 3-[4-Chloro-3-(4-fluoro-benzylsulfanyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₇H₃₂ClFN₄O₃S₂, 578.16; m/z found, 579.5 [M+H]⁺.

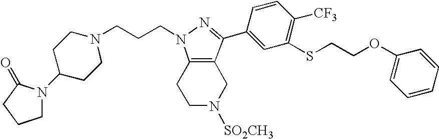

### Example 137

### 1-[1-(3-{5-Methanesulfonyl-3-[3-(2-phenoxy-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one

MS (ESI): mass calcd. for C₃₄H₄₂F₃N₅O₄S₂, 705.26; m/z found, 706.6 [M+H]⁺.

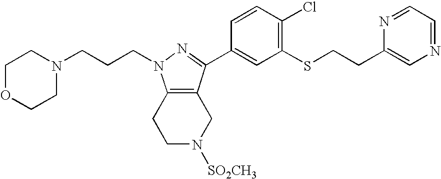

### Example 138

### 3-[4-Chloro-3-(2-pyrazin-2-yl-ethylsulfanyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₆H₃₃ClN₆O₃S₂, 576.17; m/z found, 577.5 [M+H]⁺.

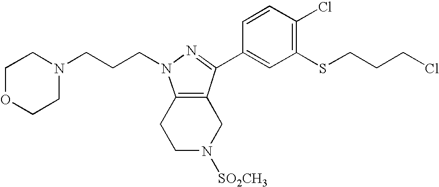

### Example 139

### 3-[4-Chloro-3-(3-chloro-propylsulfanyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₃H₃₂Cl₂N₄O₃S₂, 546.13; m/z found, 547.4 [M+H]⁺.

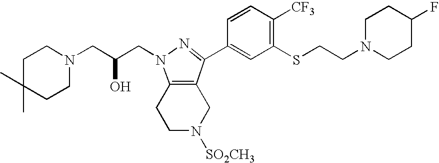

### Example 140

### (2S)-1-(4,4-Dimethyl-piperidin-1-yl)-3-(3-{3-[2-(4-fluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propan-2-ol

The title compound was prepared using methods analogous to those described for example 153, substituting (S)-(+)-glycidyl nosylate for epichlorohydrin. MS (ESI): mass calcd. for C₃₁H₄₅F₄N₅O₃S₂, 675.29; m/z found, 676.3 [M+H]⁺. ¹H NMR: 7.94 (s, 1H), 7.82 (d, J=8.2, 1H), 7.72 (d, J=8.1, 1H), 4.573 (dd, J=17.6, 14.2, 2H), 4.51-4.47 (m, 1H), 4.24 (dd, J=14.5, 4.2, 2H), 4.18 (dd, J=14.4, 6.6, 2H), 3.70-3.41 (m, 11H), 3.36 (d, J=13.3, 2.6, 1H), 3.33-3.24 (m, 2H), 3.24-3.18 (m, 2H), 3.14 (dd, J=12.5, 3.5, 1H), 3.116 (dd, J=12.4, 3.4, 1H), 3.05-2.94 (m, 5H), 2.41-1.85 (m, 4H), 1.76 (ddd, J=14.0, 13.6, 9.9, 1H), 1.71-1.58 (m, 3H), 1.08 (s, 3H), 1.03 (s, 3H).

Examples 141 and 142 were prepared using methods similar to those described in Example 140, with the appropriate substituent changes.

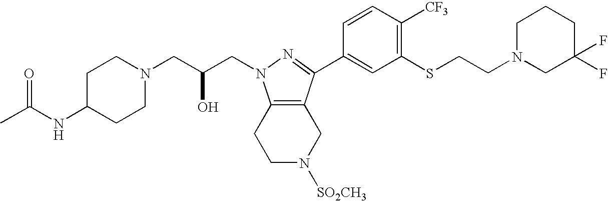

### Example 141

### (2S)—N-{1-[3-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-acetamide

MS (ESI): mass calcd. for C₃₁H₄₃F₅N₆O₄S₂, 722.27; m/z found, 723.3 [M+H]⁺. ¹H NMR: 7.95 (s, 1H), 7.81 (d, J=8.2, 1H), 7.70 (d, J=8.0, 1H), 4.57 (dd, J=17.2, 14.0, 2H), 4.53-4.47 (m, 1H), 4.41-4.14 (m, 2H), 4.07-2.92 (m, 22H), 2.41-1.65 (m, 12H).

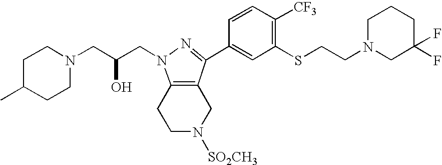

### Example 142

### (2S)-1-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-3-(4-methylpiperidin-1-yl)-propan-2-ol

MS (ESI): mass calcd. for C₃₀H₄₂F₅N₅O₃S₂, 679.26; m/z found, 680.3 [M+H]⁺. ¹H NMR: 7.95 (s, 1H), 7.81 (d, J=8.2, 1H), 7.68 (d, J=8.0, 1H), 4.58 (dd, J=17.4, 14.0, 2H), 4.55-4.48 (m, 1H), 4.25 (dd, J=14.4, 4.0, 2H), 4.19 (dd, J=14.5, 6.7, 2H), 4.01-2.80 (m, 21H), 2.28-1.36 (m, 9H), 1.25-0.88 (m, 3H).

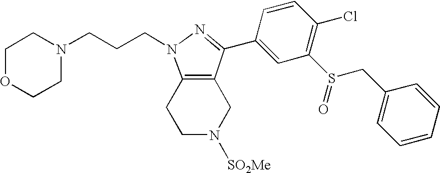

### Example 143

### 3-(4-Chloro-3-phenylmethanesulfinyl-phenyl)-5-methanesulfonyl-1-(3-morpholin-4-yl-propyl)-4,5,6,7-tetrahydro-1H-pyrazolo[4,3-c]pyridine

To a solution of 3-(3-benzylsulfanyl-4-chloro-phenyl)-5-methanesulfonyl-1-(3-morpholin-4-yl-propyl)-4,5,6,7-tetrahydro-1H-pyrazolo[4,3-c]pyridine (0.05 g, 0.07 mmol) in DMF (0.30 mL) was added tellurium dioxide (0.01 g, 0.07 mmol), 30% hydrogen peroxide (0.02 mL, 0.05 mmol), and a catalytic amount of concentrated HCl. After stirring for 16 h at rt, H₂O was added to the reaction mixture. The aqueous layer was extracted with CH₂Cl₂ (3×, 20 mL), and the combined organic layers were dried (Na₂SO₄) and concentrated to afford a bright yellow solid. Purification (SiO₂; 0-8% 1:9 satd. NH₃ in MeOH/MeOH in CH₂Cl₂) provided a yellow oil (0.03 g, 75%), and reverse phase HPLC (C₁₈; 20 mM NH₄OH/H₂O/CH₃CN) yielded the desired compound as a white powder. MS (ESI): mass calcd. for C₂₇H₃₃ClN₄O₄S₂, 576.16; m/z found, 577.5 [M+H]⁺. ¹H NMR (CDCl₃): 7.70 (dd, J=8.3, 2.2, 1H), 7.58 (d, J=2.1, 1H), 7.41 (d, J=8.3, 1H), 7.29-7.25 (m, 3H), 7.13-7.08 (m, 2H), 4.38-4.27 (m, 3H), 4.11-4.03 (m, 3H), 3.74-3.66 (m, 5H), 3.55-3.48 (m, 1H), 2.89 (s, 3H), 2.88-2.83 (m, 2H), 2.43-2.36 (m, 4H), 2.92 (t, J=6.8, 2H), 2.08-2.01 (m, 2H).

Unless otherwise specified, the compounds in Examples 144-614 were prepared as free base, hydrochloride salt, trifluoroacetic acid salt, or citric acid salt forms.

Examples 144-148 were prepared according to the methods described in Example 4, with the appropriate substituent changes.

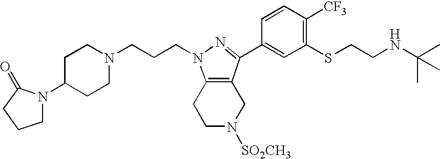

### Example 144

### 1-[1-(3-{3-[3-({2-[(1,1-Dimethylethyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one

MS (ESI): mass calcd. for C₃₂H₄₇F₃N₆O₃S₂, 684.31; m/z found, 685.3 [M+H]⁺. ¹H NMR: 7.88 (s, 1H), 7.76 (d, J=8.2, 1H), 7.63-7.59 (m, 1H), 4.46 (s, 2H), 4.21-4.14 (m, 2H), 4.04-3.96 (m, 1H), 3.62-3.52 (m, 4H), 3.48-3.44 (m, 2H), 3.30-3.08 (m, 6H), 3.05-2.96 (m, 2H), 2.89 (s, 3H), 2.88-2.84 (m, 2H), 2.31-2.20 (m, 4H), 1.98-1.80 (m, 6H), 1.26 (s, 9H).

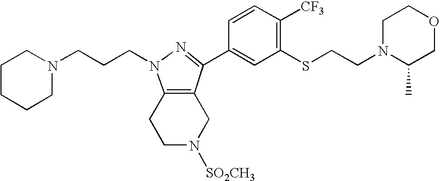

### Example 145

### 3-[3-({2-[(3S)-3-Methylmorpholin-4-yl]ethyl}sulfanyl)-4-(trifluoromethyl)phenyl]-5-(methylsulfonyl)-1-(3-piperidin-1-ylpropyl)-4,5,6,7-tetrahydro-1H-pyrazolo[4,3-c]pyridine

MS (ESI): mass calcd. for C₂₉H₄₂F₃N₅O₃S₂, 629.27; m/z found, 630.2 [M+H]⁺. ¹H NMR: 7.94 (brs, 1H), 7.75-7.67 (m, 1H), 7.62-7.54 (m, 1H), 4.61-4.38 (m, 2H), 4.24-4.11 (m, 2H), 4.02-3.72 (m, 4H), 3.68-3.33 (m, 10H), 3.16-2.71 (m, 8H), 2.40-2.21 (m, 2H), 1.89-1.65 (m, 5H), 1.49-1.14 (m, 6H).

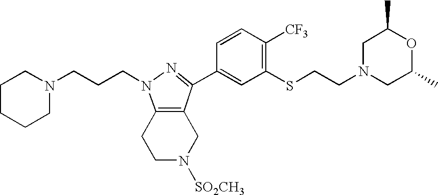

### Example 146

### 3-[3-({2-[(2R,6R)-2,6-Dimethylmorpholin-4-yl]ethyl}sulfanyl)-4-(trifluoromethyl)phenyl]-5-(methylsulfonyl)-1-(3-piperidin-1-ylpropyl)-4,5,6,7-tetrahydro-1H-pyrazolo[4,3-c]pyridine

MS (ESI): mass calcd. for C₃₀H₄₄F₃N₅O₃S₂, 643.28; m/z found, 644.3 [M+H]⁺. ¹H NMR: 7.91 (brs, 1H), 7.75-7.67 (m, 1H), 7.61-7.55 (m, 1H), 4.58-4.43 (m, 2H), 4.24-4.13 (m, 2H), 3.93-3.83 (m, 2H), 3.66-3.37 (m, 8H), 3.35-3.00 (m, 3H), 2.98-2.79 (m, 7H), 2.70-2.56 (m, 2H), 2.38-2.27 (m, 2H), 1.90-1.64 (m, 5H), 1.49-1.36 (m, 1H), 1.22-1.05 (m, 7H).

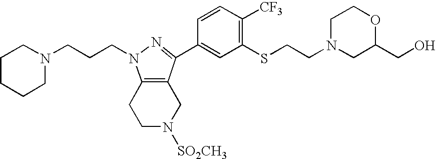

### Example 147

### {4-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]morpholin-2-yl}methanol

MS (ESI): mass calcd. for C₂₉H₄₂F₃N₅O₄S₂, 645.26; m/z found, 646.2 [M+H]⁺. ¹H NMR: 7.91 (br s, 1H), 7.74-7.67 (m, 1H), 7.60-7.54 (m, 1H), 4.59-4.43 (m, 2H), 4.24-4.14 (m, 2H), 4.06-3.98 (m, 1H), 3.88-3.75 (m, 2H), 3.67-3.29 (m, 12H), 3.15-2.74 (m, 10H), 2.44-2.18 (m, 2H), 1.90-1.65 (m, 5H), 1.47-1.38 (m, 1H), 1.30-1.13 (m, 1H).

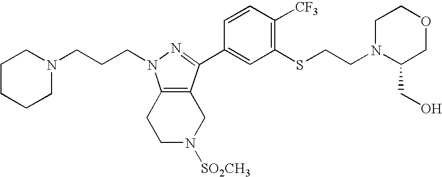

### Example 148

### {(3S)-4-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]morpholin-3-yl}methanol

MS (ESI): mass calcd. for C₂₉H₄₂F₃N₅O₄S₂, 645.26; m/z found, 646.2 [M+H]⁺. ¹H NMR: 7.95 (br s, 1H), 7.78-7.69 (m, 1H), 7.64-7.58 (m, 1H), 4.63-4.40 (m, 2H), 4.28-4.14 (m, 2H), 4.04-3.34 (m, 14H), 3.17-2.73 (m, 9H), 2.43-2.21 (m, 2H), 1.92-1.68 (m, 5H), 1.52-1.37 (m, 2H), 1.35-1.15 (m, 2H).

Examples 149-152 were prepared according to the methods described in Example 54, with the appropriate substituent changes.

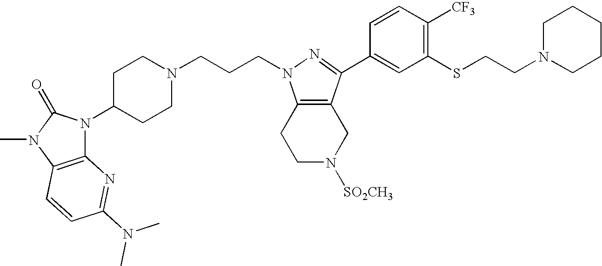

### Example 149

### 5-(Dimethylamino)-1-methyl-3-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-imidazo[4,5-b]pyridin-2-one

MS (ESI): mass calcd. for C₃₈H₅₂F₃N₉O₃S₂, 803.36; m/z found, 804.3 [M+H]⁺. ¹H NMR: 7.91 (s, 1H), 7.72 (d, J=8.2, 1H), 7.53 (d, J=8.2, 1H), 7.27-7.23 (m, 1H), 6.31 (d, J=8.6, 1H), 4.56 (s, 2H), 4.33-4.23 (m, 1H), 4.21-4.16 (m, 2H), 3.69-3.64 (m, 3H), 3.56-3.53 (m, 1H), 3.24-3.19 (m, 2H), 3.09-2.97 (m, 10H), 2.96 (s, 3H), 2.83-2.72 (m, 2H), 2.66-2.60 (m, 2H), 2.47 (br s, 4H), 2.41-2.35 (m, 2H), 2.15-2.06 (m, 4H), 1.73-1.66 (m, 2H), 1.61-1.37 (m, 7H).

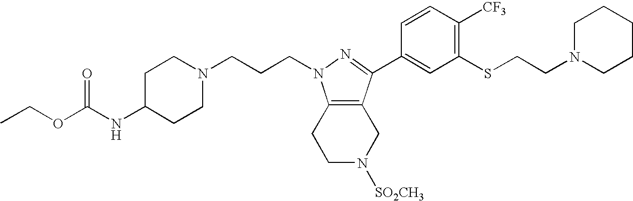

### Example 150

### Ethyl(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate

MS (ESI): mass calcd. for C₃₂H₄₇F₃N₆O₄S₂, 700.31; m/z found, 701.3 [M+H]⁺. ¹H NMR: 7.93 (s, 1H), 7.82-7.79 (m, 1H), 7.72-7.66 (m, 1H), 4.57 (s, 2H), 4.32-4.18 (m, 2H), 4.16-4.02 (m, 2H), 3.75-3.55 (m, 7H), 3.54-3.42 (m, 3H), 3.41-3.31 (m, 3H), 3.29-3.15 (m, 3H), 3.13-2.90 (m, 5H), 2.43-2.28 (m, 2H), 2.21-2.10 (m, 2H), 2.09-1.99 (m, 1H), 1.98-1.88 (m, 2H), 1.88-1.69 (m, 5H), 1.58-1.42 (m, 1H), 1.31-1.15 (m, 3H).

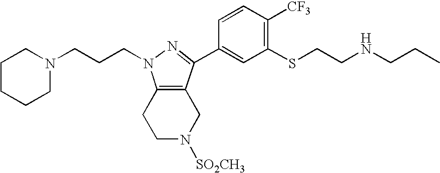

### Example 151

### N-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]propan-1-amine

MS (ESI): mass calcd. for C₂₇H₄₀F₃N₅O₂S₂, 587.26; m/z found, 588.3 [M+H]⁺. ¹H NMR: 7.97 (s, 1H), 7.83 (d, J=8.2, 1H), 7.72-7.68 (m, 1H), 4.57 (s, 2H), 4.31-4.21 (m, 2H), 3.74-3.65 (m, 2H), 3.61-3.51 (m, 2H), 3.43-3.25 (m, 4H), 3.23-3.15 (m, 2H), 3.09-2.88 (m, 9H), 2.42-2.29 (m, 2H), 2.00-1.89 (m, 2H), 1.87-1.63 (m, 5H), 1.58-1.42 (m, 1H), 1.01 (t, J=7.4, 3H).

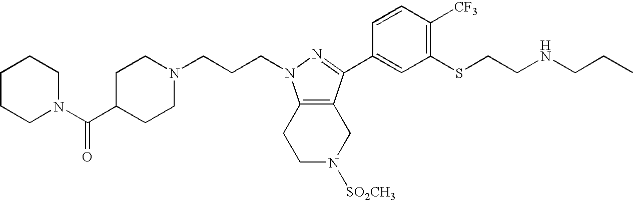

### Example 152

### N-[2-({5-[5-(Methylsulfonyl)-1-{3-[4-(piperidin-1-ylcarbonyl)piperidin-1-yl]propyl}-4,5,6,7-tetrahydro-1H-pyrazolo[4,3-c]pyridin-3-yl]-2-(trifluoromethyl)phenyl}sulfanyl)ethyl]propan-1-amine

MS (ESI): mass calcd. for C₃₃H₄₉F₃N₆O₃S₂, 698.33; m/z found, 699.3 [M+H]⁺. ¹H NMR: 7.86 (s, 1H), 7.74 (d, J=8.3, 1H), 7.63-7.59 (m, 1H), 4.46 (s, 2H), 4.20-4.12 (m, 2H), 3.64-3.49 (m, 4H), 3.49-3.38 (m, 4H), 3.33-3.04 (m, 6H), 2.99-2.82 (m, 10H), 2.32-2.19 (m, 2H), 1.97-1.76 (m, 4H), 1.65-1.55 (m, 4H), 1.54-1.47 (m, 2H), 1.46-1.40 (m, 2H), 0.90 (t, J=7.4, 3H).

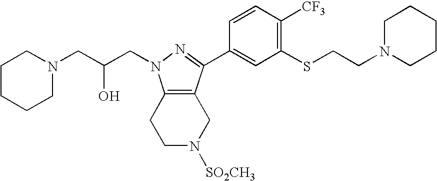

### Example 153

### 1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol

A. 2-Piperidin-1-yl-ethanethiol. To a stirring solution of piperidine (3.3 mL, 34 mmol) in toluene (18 mL) was added ethylene sulfide. A reflux condenser was attached, the flask was purged with N₂, and the mixture was heated at reflux for 4 h. The mixture was cooled to rt and concentrated to afford the title compound (3.3 g, 58%), which was used immediately without further purification.

B. 5-Methanesulfonyl-3-[3-(2-piperidin-1-yl-ethylsulfanyl)-4-trifluoromethyl-phenyl]-4,5,6,7-tetrahydro-1H-pyrazolo[4,3-c]pyridine. This compound was prepared using methods similar to those described in Example 1, Step C, substituting 2-piperidin-1-yl-ethanethiol for 2-mercaptoethanol.

C. 1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol. This compound was prepared using methods similar to those described in Example 61, Steps B and C, substituting piperidine for 1-piperidin-4-yl-pyrrolidin-2-one. Purification was conducted using reverse phase HPLC. MS (ESI): mass calcd. for C₂₉H₄₂F₃N₅O₃S₂, 629.27; m/z found, 630.2 [M+H]⁺. ¹H NMR (CDCl₃): 7.83 (s, 1H), 7.66 (d, J=8.1, 1H), 7.38 (d, J=8.0, 1H), 4.59 (d, J=14.5, 1H), 4.53 (d, J=14.4, 1H), 4.18 (dd, J=14.1, 2.7, 1H), 4.13-4.07 (m, 1H), 3.99 (dd, J=14.0, 6.9, 1H), 3.75-3.69 (m, 1H), 3.68-3.60 (m, 1H), 3.17 (dd, J=8.9, 6.7, 2H), 3.14-3.07 (m, 1H), 2.96-2.89 (m, 1H), 2.87 (s, 3H), 2.65-2.61 (m, 2H), 2.61-2.51 (m, 2H), 2.51-2.37 (m, 5H), 2.37-2.26 (m, 2H), 1.61-1.50 (m, 10H), 1.48-1.39 (m, 4H).

Examples 154-189 were prepared using methods similar to those described in Example 153, with the appropriate substituent changes.

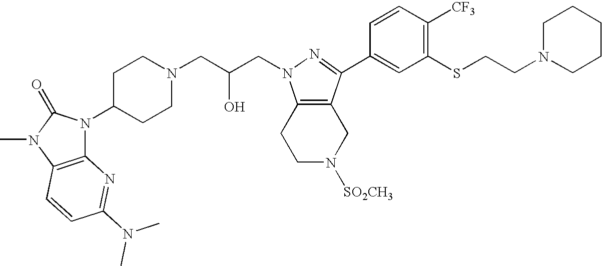

### Example 154

### 5-(Dimethylamino)-3-(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1-methyl-1,3-dihydro-2H-imidazo[4,5-b]pyridin-2-one

MS (ESI): mass calcd. for C₃₈H₅₂F₃N₉O₄S₂, 819.35; m/z found, 820.3 [M+H]⁺. ¹H NMR: 8.01 (s, 1H), 7.81 (d, J=7.8, 1H), 7.70 (d, J=6.8, 1H), 7.62 (d, J=7.7, 1H), 7.31 (d, J=7.7, 1H), 4.81-4.73 (m, 1H), 4.73-4.66 (m, 1H), 4.65-4.56 (m, 2H), 4.35-4.17 (m, 2H), 3.87 (d, J=29.1, 2H), 3.74-3.66 (m, 2H), 3.67-3.53 (m, 5H), 3.51-3.40 (m, 6H), 3.40-3.33 (m, 8H), 3.19-2.90 (m, 10H), 2.19-2.01 (m, 2H), 1.98-1.89 (m, 2H), 1.88-1.77 (m, 3H), 1.57-1.44 (m, 1H).

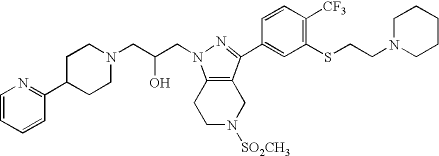

### Example 155

### 1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol

MS (ESI): mass calcd. for C₃₄H₄₅F₃N₆O₃S₂, 706.29; m/z found, 707.3 [M+H]⁺. ¹H NMR: 8.80 (s, 1H), 8.63 (s, 1H), 8.11-8.04 (m, 1H), 8.03-7.98 (m, 2H), 7.84-7.76 (m, 1H), 7.73-7.65 (m, 1H), 4.71-4.62 (m, 1H), 4.62-4.55 (m, 1H), 4.41-4.11 (m, 2H), 3.96-3.76 (m, 2H), 3.76-3.39 (m, 10H), 3.38-3.31 (m, 4H), 3.12-2.82 (m, 6H), 2.44-2.20 (m, 4H), 2.19-2.07 (m, 1H), 1.99-1.88 (m, 2H), 1.88-1.76 (m, 2H), 1.58-1.44 (m, 1H), 1.44-1.23 (m, 2H).

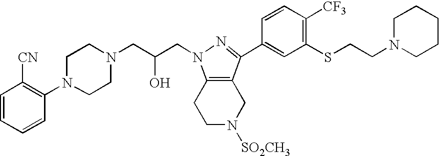

### Example 156

### 2-(4-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile

MS (ESI): mass calcd. for C₃₅H₄₄F₃N₇O₃S₂, 731.29; m/z found, 732.2 [M+H]⁺. ¹H NMR: 7.95 (s, 1H), 7.83 (d, J=8.3, 1H), 7.75 (d, J=8.1, 1H), 7.69 (d, J=7.7, 1H), 7.64 (dd, J=8.3, 7.6, 1H), 7.24-7.19 (m, 2H), 4.64-4.51 (m, 3H), 4.29 (dd, J=14.4, 4.1, 1H), 4.22 (dd, J=14.5, 6.5, 1H), 3.72-3.62 (m, 5H), 3.61-3.54 (m, 4H), 3.52-3.44 (m, 5H), 3.40-3.33 (m, 4H), 3.06-3.01 (m, 3H), 3.00-2.98 (m, 5H), 1.99-1.88 (m, 2H), 1.87-1.67 (m, 3H), 1.55-1.42 (m, 1H).

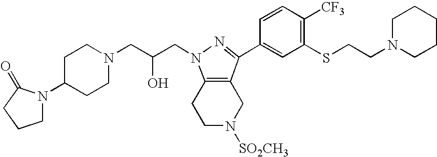

### Example 157

### 1-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one

MS (ESI): mass calcd. for C₃₃H₄₇F₃N₆O₄S₂, 712.31; m/z found, 713.3 [M+H]⁺. ¹H NMR: 7.99 (s, 1H), 7.80 (d, J=7.7, 1H), 7.72-7.65 (m, 1H), 4.65-4.45 (m, 3H), 4.31-4.05 (m, 4H), 3.83-3.62 (m, 4H), 3.62-3.51 (m, 5H), 3.44-3.39 (m, 2H), 3.38-3.32 (m, 3H), 3.05-2.91 (m, 8H), 2.37 (t, J=7.9, 2H), 2.22-2.10 (m, 1H), 2.10-1.98 (m, 3H), 1.98-1.87 (m, 4H), 1.86-1.76 (m, 4H), 1.56-1.45 (m, 1H).

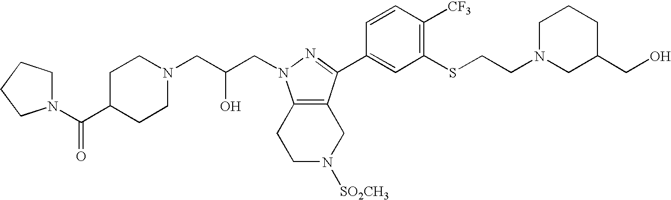

### Example 158

### 1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol

MS (ESI): mass calcd. for C₃₅H₅₁F₃N₆O₅S₂, 756.33; m/z found, 757.3 [M+H]⁺. ¹H NMR: 7.96 (s, 1H), 7.84 (d, J=8.3, 1H), 7.73 (d, J=8.2, 1H), 4.63-4.46 (m, 3H), 4.27 (dd, J=14.3, 3.3, 2H), 4.20 (dd, J=14.4, 6.5, 2H), 3.79-3.34 (m, 18H), 3.29-3.04 (m, 3H), 3.05-2.95 (m, 5H), 2.97-2.80 (m, 2H), 2.80-2.72 (m, 1H), 2.09-1.94 (m, 7H), 1.91 (dd, J=13.7, 6.8, 2H), 1.87-1.75 (m, 2H), 1.29 (d, J=12.4, 1H).

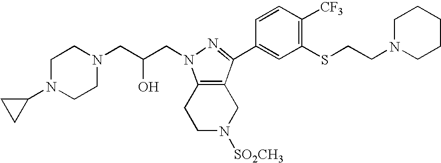

### Example 159

### 1-(4-Cyclopropylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol

MS (ESI): mass calcd. for C₃₁H₄₅F₃N₆O₃S₂, 670.29; m/z found, 671.3 [M+H]⁺. ¹H NMR: 8.08-7.94 (m, 1H), 7.80 (d, J=7.5, 1H), 7.67 (d, J=7.5, 1H), 4.71-4.61 (m, 1H), 4.61-4.50 (m, 2H), 4.32-4.21 (m, 2H), 3.98-3.73 (m, 7H), 3.72-3.49 (m, 7H), 3.37-3.31 (m, 3H), 3.08-2.92 (m, 8H), 1.93 (d, J=11.9, 2H), 1.82 (d, J=10.7, 3H), 1.57-1.43 (m, 1H), 1.28-1.21 (m, 3H), 1.04-0.96 (m, 2H), 0.95-0.86 (m, 1H).

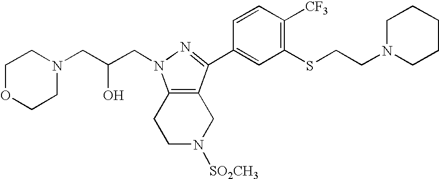

### Example 160

### 1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-morpholin-4-ylpropan-2-ol

MS (ESI): mass calcd. for C₂₈H₄₀F₃N₅O₄S₂, 631.25; m/z found, 632.2 [M+H]⁺. ¹H NMR (CDCl₃): 7.83 (s, 1H), 7.66 (d, J=8.2, 1H), 7.38 (d, J=8.2, 1H), 4.55 (q, J=14.3, 2H), 4.20 (dd, J=14.0, 2.9, 1H), 4.17-4.12 (m, 1H), 4.01 (dd, J=14.0, 6.8, 1H), 3.77-3.59 (m, 7H), 3.18-3.13 (m, 2H), 3.10-3.03 (m, 1H), 2.97-2.89 (m, 1H), 2.88 (s, 3H), 2.69-2.58 (m, 4H), 2.51-2.35 (m, 8H), 1.59-1.55 (m, 4H), 1.46-1.37 (m, 2H).

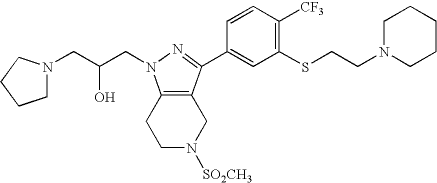

### Example 161

### 1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-pyrrolidin-1-ylpropan-2-ol

MS (ESI): mass calcd. for C₂₈H₄₀F₃N₅O₃S₂, 615.25; m/z found, 616.2 [M+H]⁺. ¹H NMR (CDCl₃): 7.83 (s, 1H), 7.66 (d, J=8.2, 1H), 7.39 (d, J=8.1, 1H), 4.56 (q, J=14.5, 2H), 4.20 (dd, J=13.9, 2.8, 1H), 4.12-4.06 (m, 1H), 4.02 (dd, J=13.9, 7.0, 1H), 3.74-3.69 (m, 1H), 3.69-3.60 (m, 1H), 3.16 (dd, J=8.8, 6.8, 2H), 3.10-3.04 (m, 1H), 2.96-2.89 (m, 1H), 2.87 (s, 3H), 2.68-2.58 (m, 5H), 2.53-2.47 (m, 2H), 2.44 (dd, J=11.9, 4.3, 5H), 1.78 (t, J=6.3, 4H), 1.70-1.62 (m, 3H), 1.59-1.54 (m, 4H).

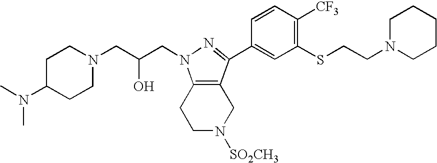

### Example 162

### 1-[4-(Dimethylamino)piperidin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol

MS (ESI): mass calcd. for C₃₁H₄₇F₃N₆O₃S₂, 672.31; m/z found, 673.3 [M+H]⁺. ¹H NMR (CDCl₃): 7.83 (s, 1H), 7.66 (d, J=8.2, 1H), 7.38 (d, J=8.3, 1H), 4.58 (d, J=15.4, 1H), 4.53 (d, J=14.5, 1H), 4.18 (dd, J=14.0, 2.7, 1H), 4.13-4.06 (m, 1H), 4.00 (dd, J=14.0, 7.0, 1H), 3.76-3.67 (m, 1H), 3.67-3.61 (m, 1H), 3.19-3.13 (m, 2H), 3.00-2.93 (m, 2H), 2.88-2.86 (m, 3H), 2.85-2.81 (m, 1H), 2.65-2.62 (m, 2H), 2.48-2.34 (m, 5H), 2.27-2.26 (m, 5H), 2.13-2.05 (m, 1H), 2.04-1.96 (m, 1H), 1.86-1.78 (m, 3H), 1.76-1.62 (m, 7H), 1.60-1.54 (m, 5H).

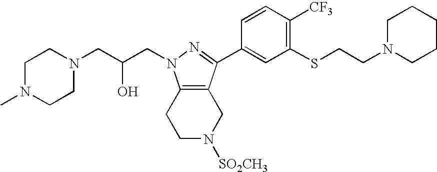

### Example 163

### 1-(4-Methylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol

MS (ESI): mass calcd. for C₂₉H₄₃F₃N₆O₃S₂, 644.28; m/z found, 645.2 [M+H]⁺. ¹H NMR (CDCl₃): 7.83 (s, 1H), 7.66 (d, J=8.2, 1H), 7.38 (d, J=8.0, 1H), 4.58 (d, J=14.4, 1H), 4.53 (d, J=14.5, 1H), 4.19 (dd, J=14.1, 2.9, 1H), 4.16-4.09 (m, 1H), 4.00 (dd, J=14.1, 7.0, 1H), 3.85-3.73 (m, 1H), 3.73-3.69 (m, 1H), 3.68-3.61 (m, 1H), 3.18-3.14 (m, 2H), 3.11-3.05 (m, 1H), 2.96-2.89 (m, 1H), 2.87 (s, 3H), 2.74-2.58 (m, 5H), 2.54-2.34 (m, 11H), 2.28 (s, 3H), 1.59-1.55 (m, 4H), 1.46-1.37 (m, 2H).

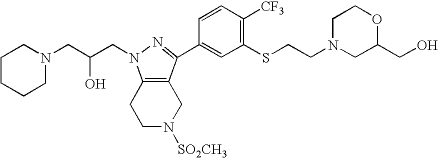

### Example 164

### 1-{3-[3-({2-[2-(Hydroxymethyl)morpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

MS (ESI): mass calcd. for C₂₉H₄₂F₃N₅O₅S₂, 661.26; m/z found, 662.3 [M+H]⁺. ¹H NMR: 7.94 (s, 1H), 7.85-7.81 (m, 1H), 7.76-7.72 (m, 1H), 4.62-4.54 (m, 2H), 4.51-4.46 (m, 1H), 4.27-4.10 (m, 3H), 3.85-3.73 (m, 2H), 3.71-3.44 (m, 13H), 3.22-3.09 (m, 2H), 3.08-2.93 (m, 8H), 1.98-1.67 (m, 5H), 1.58-1.48 (m, 1H).

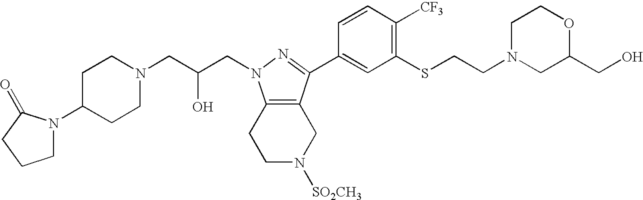

### Example 165

### 1-[1-(2-Hydroxy-3-{3-[3-({2-[2-(hydroxymethyl)morpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one

MS (ESI): mass calcd. for C₃₃H₄₇F₃N₆O₆S₂, 744.30; m/z found, 745.3 [M+H]⁺. ¹H NMR: 7.94 (s, 1H), 7.85-7.82 (m, 1H), 7.76-7.73 (m, 1H), 4.62-4.53 (m, 2H), 4.52-4.45 (m, 1H), 4.29-4.08 (m, 4H), 3.86-3.38 (m, 17H), 3.29-3.08 (m, 4H), 3.06-2.96 (m, 6H), 2.42-2.35 (m, 2H), 2.18-1.88 (m, 6H).

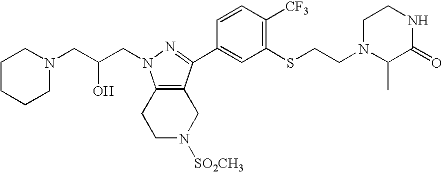

### Example 166

### 4-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl}sulfanyl)ethyl]-3-methylpiperazin-2-one

MS (ESI): mass calcd. for C₂₉H₄₁F₃N₆O₄S₂, 658.26; m/z found, 659.2 [M+H]⁺. ¹H NMR: 8.02 (s, 1H), 7.82 (d, J=8.2, 1H), 7.66 (d, J=8.1, 1H), 4.65-4.49 (m, 3H), 4.30-4.14 (m, 2H), 3.93-3.84 (m, 1H), 3.72-3.27 (m, 13H), 3.24-3.15 (m, 1H), 3.12-2.95 (m, 7H), 2.03-1.72 (m, 5H), 1.61-1.49 (m, 4H).

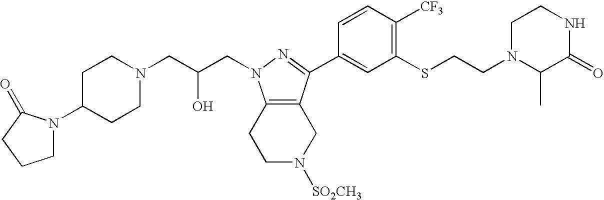

### Example 167

### 4-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]-3-methylpiperazin-2-one

MS (ESI): mass calcd. for C₃₃H₄₆F₃N₇O₅S₂, 741.30; m/z found, 742.3 [M+H]⁺. ¹H NMR: 8.01 (s, 1H), 7.85-7.81 (m, 1H), 7.69-7.65 (m, 1H), 4.59 (s, 2H), 4.54-4.48 (m, 1H), 4.31-4.08 (m, 3H), 3.88-3.59 (m, 5H), 3.56-3.37 (m, 9H), 3.30-3.11 (m, 5H), 3.05-2.95 (m, 5H), 2.40 (t, J=8.1, 2H), 2.18-1.90 (m, 6H), 1.50 (t, J=7.0, 3H).

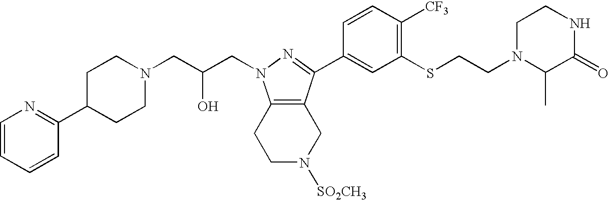

### Example 168

### 4-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)-3-methylpiperazin-2-one

MS (ESI): mass calcd. for C₃₄H₄₄F₃N₇O₄S₂, 735.28; m/z found, 736.3 [M+H]⁺. ¹H NMR: 8.62-8.58 (m, 1H), 8.06-7.98 (m, 2H), 7.82 (d, J=8.2, 1H), 7.67 (d, J=8.1, 1H), 7.57-7.46 (m, 2H), 4.64-4.51 (m, 3H), 4.31-4.17 (m, 2H), 3.91-3.74 (m, 3H), 3.72-3.60 (m, 2H), 3.59-3.53 (m, 1H), 3.53-3.26 (m, 10H), 3.24-3.13 (m, 2H), 3.05-2.97 (m, 5H), 2.28-2.05 (m, 4H), 1.55-1.47 (m, 3H).

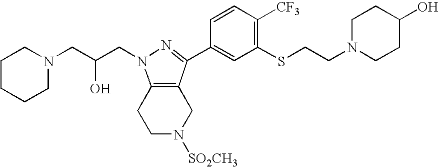

### Example 169

### 1-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol

MS (ESI): mass calcd. for C₂₉H₄₂F₃N₅O₄S₂, 645.26; m/z found, 646.2 [M+H]⁺. ¹H NMR: 7.93 (d, J=11.3, 1H), 7.82 (d, J=8.2, 1H), 7.76-7.69 (m, 1H), 4.61-4.53 (m, 2H), 4.52-4.46 (m, 1H), 4.27-4.14 (m, 2H), 4.10-4.06 (m, 0.5H), 3.83-3.75 (m, 0.5H), 3.71-3.51 (m, 5H), 3.51-3.30 (m, 7H), 3.22-3.15 (m, 1H), 3.11-2.92 (m, 8H), 2.17-2.09 (m, 1H), 2.02-1.65 (m, 8H), 1.58-1.45 (m, 1H).

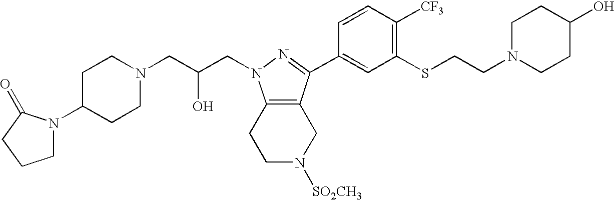

### Example 170

### 1-[1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one

MS (ESI): mass calcd. for C₃₃H₄₇F₃N₆O₅S₂, 728.30; m/z found, 729.3 [M+H]⁺. ¹H NMR: 7.97-7.91 (m, 1H), 7.84-7.81 (m, 1H), 7.78-7.71 (m, 1H), 4.59-4.56 (m, 2H), 4.52-4.46 (m, 1H), 4.29-4.05 (m, 4H), 3.84-3.56 (m, 5H), 3.51-3.29 (m, 9H), 3.28-3.03 (m, 4H), 3.02-2.94 (m, 5H), 2.39 (t, J=8.1, 2H), 2.17-2.09 (m, 2H), 2.08-2.00 (m, 3H), 1.99-1.88 (m, 4H), 1.74-1.64 (m, 1H).

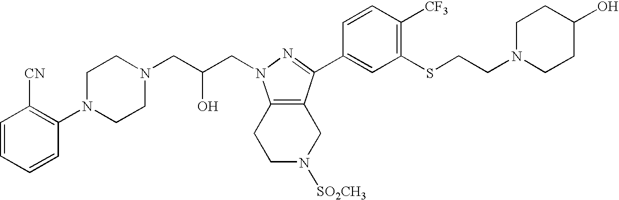

### Example 171

### 2-[4-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperazin-1-yl]benzonitrile

MS (ESI): mass calcd. for C₃₅H₄₄F₃N₇O₄S₂, 747.28; m/z found, 748.2 [M+H]⁺. ¹H NMR: 8.00-7.91 (m, 1H), 7.86-7.82 (m, 1H), 7.79-7.73 (m, 1H), 7.71-7.68 (m, 1H), 7.66-7.62 (m, 1H), 7.27-7.18 (m, 2H), 4.64-4.51 (m, 3H), 4.33-4.20 (m, 2H), 3.88-3.26 (m, 20H), 3.12-2.92 (m, 6H), 2.20-2.07 (m, 1H), 2.02-1.87 (m, 2H), 1.75-1.61 (m, 1H).

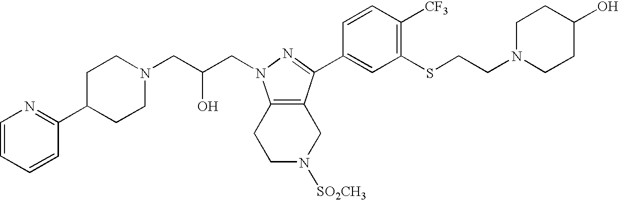

### Example 172

### 1-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-ol

MS (ESI): mass calcd. for C₃₄H₄₅F₃N₆O₄S₂, 722.29; m/z found, 723.3 [M+H]⁺. ¹H NMR: 8.69-8.61 (m, 1H), 8.19-8.11 (m, 1H), 7.98-7.90 (m, 1H), 7.85-7.80 (m, 1H), 7.77-7.70 (m, 1H), 7.67-7.57 (m, 2H), 4.64-4.50 (m, 3H), 4.32-4.18 (m, 2H), 4.11-4.05 (m, 0.5H), 3.86-3.74 (m, 2.5H), 3.72-3.60 (m, 3H), 3.54-3.16 (m, 11H), 3.13-2.93 (m, 6H), 2.31-2.07 (m, 5H), 2.01-1.88 (m, 2H), 1.77-1.62 (m, 1H).

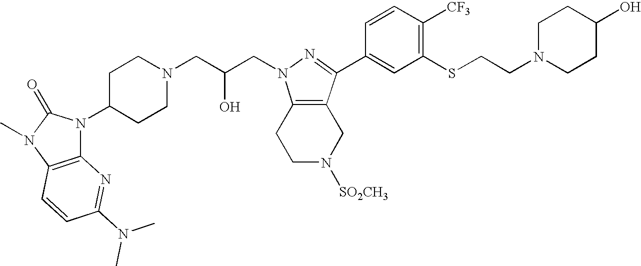

### Example 173

### 5-(Dimethylamino)-3-[1-(2-hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one

MS (ESI): mass calcd. for C₃₈H₅₂F₃N₉O₅S₂, 835.35; m/z found, 836.3 [M+H]⁺. ¹H NMR: 8.02-7.71 (m, 3H), 7.38-7.29 (m, 1H), 6.46-6.31 (m, 1H), 4.76-4.49 (m, 4H), 4.36-4.19 (m, 2H), 3.89-3.58 (m, 6H), 3.56-3.19 (m, 12H), 3.16-2.94 (m, 15H), 2.24-1.86 (m, 5H), 1.81-1.58 (m, 1H).

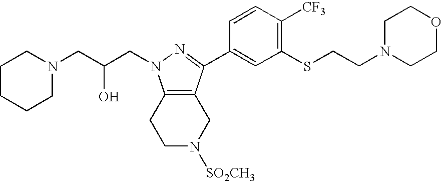

### Example 174

### 1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol

MS (ESI): mass calcd. for C₂₈H₄₀F₃N₅O₄S₂, 631.25; m/z found, 632.2 [M+H]⁺. ¹H NMR: 8.01 (s, 1H), 7.86-7.81 (m, 1H), 7.74-7.68 (m, 1H), 4.69-4.54 (m, 3H), 4.33-4.15 (m, 2H), 4.11-4.01 (m, 2H), 3.93-3.82 (m, 2H), 3.75-3.54 (m, 8H), 3.53-3.33 (m, 3H), 3.27-3.17 (m, 3H), 3.16-2.96 (m, 7H), 2.05-1.74 (m, 5H), 1.63-1.49 (m, 1H).

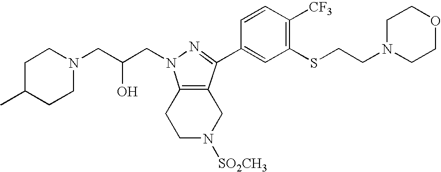

### Example 175

### 1-(4-Methylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol

MS (ESI): mass calcd. for C₂₉H₄₂F₃N₅O₄S₂, 645.26; m/z found, 646.3 [M+H]⁺. ¹H NMR: 7.94 (s, 1H), 7.87-7.82 (m, 1H), 7.79-7.74 (m, 1H), 4.60 (s, 2H), 4.54-4.47 (m, 1H), 4.30-4.15 (m, 2H), 3.92 (br s, 4H), 3.74-3.38 (m, 10H), 3.32-3.16 (m, 4H), 3.12-2.91 (m, 7H), 1.99-1.81 (m, 2H), 1.77-1.65 (m, 1H), 1.60-1.37 (m, 2H), 1.06-0.95 (m, 3H).

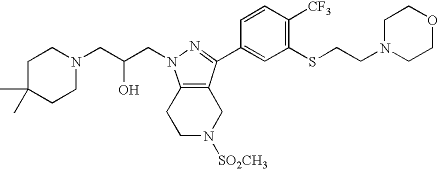

### Example 176

### 1-(4,4-Dimethylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol

MS (ESI): mass calcd. for C₃₀H₄₄F₃N₅O₄S₂, 659.28; m/z found, 660.3 [M+H]⁺. ¹H NMR: 7.93 (s, 1H), 7.85-7.81 (m, 1H), 7.77-7.73 (m, 1H), 4.61-4.55 (m, 2H), 4.51-4.46 (m, 1H), 4.28-4.15 (m, 2H), 4.13-3.72 (m, 4H), 3.74-3.58 (m, 2H), 3.53-3.40 (m, 7H), 3.39-3.28 (m, 3H), 3.26-3.07 (m, 4H), 3.04-2.94 (m, 5H), 1.84-1.53 (m, 4H), 1.13-0.99 (m, 6H).

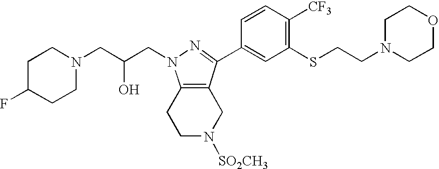

### Example 177

### 1-(4-Fluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol

MS (ESI): mass calcd. for C₂₈H₃₉F₄N₅O₄S₂, 649.24; m/z found, 650.2 [M+H]⁺. ¹H NMR: 7.93 (s, 1H), 7.84-7.81 (m, 1H), 7.76-7.73 (m, 1H), 4.59-4.56 (m, 2H), 4.55-4.47 (m, 1H), 4.27-4.15 (m, 2H), 4.11-3.73 (m, 3H), 3.72-3.60 (m, 3H), 3.59-3.33 (m, 9H), 3.28-3.18 (m, 3H), 3.03-2.95 (m, 5H), 2.33-1.92 (m, 4H).

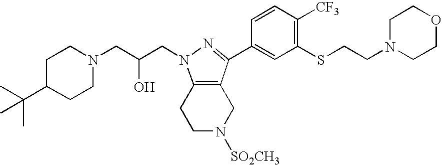

### Example 178

### 1-[4-(1,1-Dimethylethyl)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol

MS (ESI): mass calcd. for C₃₂H₄₈F₃N₅O₄S₂, 687.31; m/z found, 688.3 [M+H]⁺. ¹H NMR: 7.83 (s, 1H), 7.75-7.72 (m, 1H), 7.67-7.65 (m, 1H), 4.54-4.45 (m, 2H), 4.43-4.36 (m, 1H), 4.18-4.04 (m, 2H), 4.03-3.64 (m, 4H), 3.64-3.50 (m, 5H), 3.48-3.04 (m, 10H), 3.00-2.78 (m, 6H), 1.97-1.79 (m, 2H), 1.60-1.39 (m, 2H), 1.34-1.23 (m, 1H), 0.81 (s, 9H).

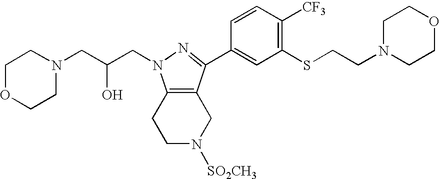

### Example 179

### 1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-morpholin-4-ylpropan-2-ol

MS (ESI): mass calcd. for C₂₇H₃₈F₃N₅O₅S₂, 633.22; m/z found, 634.2 [M+H]⁺. ¹H NMR: 7.92 (s, 1H), 7.72 (d, J=8.2, 1H), 7.58 (d, J=8.1, 1H), 4.60-4.44 (m, 3H), 4.24-4.09 (m, 2H), 4.01-3.87 (m, 4H), 3.84-3.67 (m, 4H), 3.65-3.41 (m, 9H), 3.39-3.29 (m, 3H), 3.29-3.07 (m, 4H), 3.00-2.84 (m, 5H).

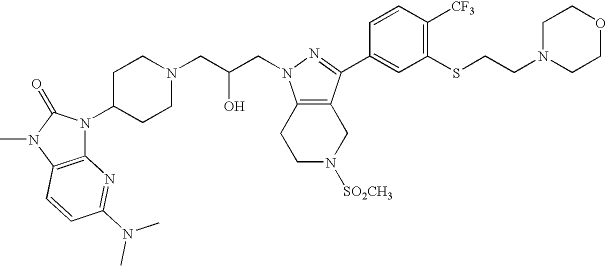

### Example 180

### 5-(Dimethylamino)-3-(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1-methyl-1,3-dihydro-2H-imidazo[4,5-b]pyridin-2-one

MS (ESI): mass calcd. for C₃₇H₅₀F₃N₉O₅S₂, 821.33; m/z found, 822.3 [M+H]⁺. ¹H NMR: 8.04 (s, 1H), 7.87-7.80 (m, 1H), 7.75-7.62 (m, 2H), 7.40-7.35 (m, 1H), 4.84-4.70 (m, 2H), 4.69-4.58 (m, 2H), 4.41-4.21 (m, 2H), 4.13-4.00 (m, 2H), 3.97-3.80 (m, 4H), 3.78-3.54 (m, 8H), 3.52-3.29 (m, 11H), 3.28-3.20 (m, 3H), 3.19-2.95 (m, 8H), 2.20-2.03 (m, 2H).

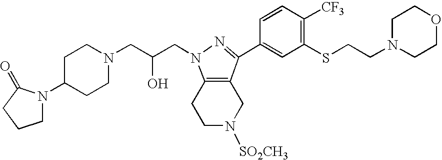

### Example 181

### 1-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one

MS (ESI): mass calcd. for C₃₂H₄₅F₃N₆O₅S₂, 714.28; m/z found, 715.2 [M+H]⁺. ¹H NMR: 7.84 (s, 1H), 7.75-7.71 (m, 1H), 7.67-7.64 (m, 1H), 4.54-4.44 (m, 2H), 4.44-4.35 (m, 1H), 4.19-3.98 (m, 4H), 3.66-3.50 (m, 6H), 3.48-3.44 (m, 2H), 3.41-3.29 (m, 8H), 3.26-2.98 (m, 5H), 2.94-2.86 (m, 5H), 2.29 (t, J=8.1, 2H), 2.08-1.98 (m, 1H), 1.98-1.90 (m, 3H), 1.88-1.77 (m, 2H).

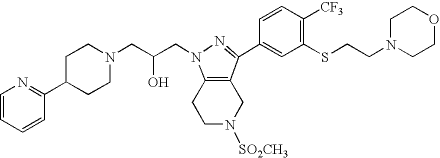

### Example 182

### 1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol

MS (ESI): mass calcd. for C₃₃H₄₃F₃N₆O₄S₂, 708.27; m/z found, 709.2 [M+H]⁺. ¹H NMR: 8.86-8.81 (m, 1H), 8.70-8.63 (m, 1H), 8.10 (d, J=8.2, 1H), 8.07-8.01 (m, 2H), 7.87-7.81 (m, 1H), 7.73-7.68 (m, 1H), 4.74-4.67 (m, 1H), 4.66-4.59 (m, 2H), 4.37-4.25 (m, 2H), 4.09-4.03 (m, 2H), 3.94-3.83 (m, 3H), 3.74-3.39 (m, 12H), 3.36 (s, 3H), 3.28-3.19 (m, 2H), 3.09-3.00 (m, 5H), 2.43-2.23 (m, 4H).

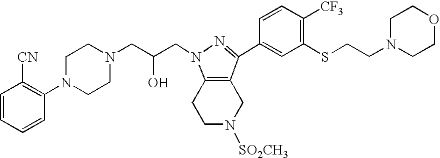

### Example 183

### 2-(4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile

MS (ESI): mass calcd. for C₃₄H₄₂F₃N₇O₄S₂, 733.27; m/z found, 734.2 [M+H]⁺. ¹H NMR: 7.96 (s, 1H), 7.87-7.83 (m, 1H), 7.79-7.76 (m, 1H), 7.72-7.69 (m, 1H), 7.67-7.63 (m, 1H), 7.27-7.19 (m, 2H), 4.64-4.52 (m, 3H), 4.33-4.21 (m, 2H), 4.15-3.77 (m, 5H), 3.75-3.60 (m, 7H), 3.53-3.30 (m, 12H), 3.07-2.97 (m, 5H).

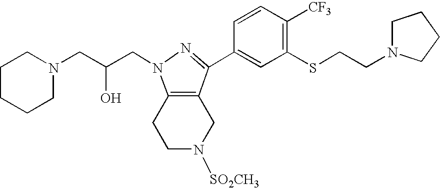

### Example 184

### 1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol

MS (ESI): mass calcd. for C₂₈H₄₀F₃N₅O₃S₂, 615.25; m/z found, 616.2 [M+H]⁺. ¹H NMR: 7.94 (s, 1H), 7.83 (d, J=8.3, 1H), 7.74 (d, J=8.1, 1H), 4.57 (d, J=2.5, 2H), 4.52-4.46 (m, 1H), 4.27-4.14 (m, 2H), 3.79-3.61 (m, 4H), 3.60-3.53 (m, 2H), 3.50-3.41 (m, 4H), 3.36-3.32 (m, 1H), 3.21-3.15 (m, 1H), 3.15-3.02 (m, 3H), 3.02-2.99 (m, 2H), 2.98 (s, 3H), 2.97-2.92 (m, 1H), 2.24-2.08 (m, 2H), 2.07-1.98 (m, 2H), 1.97-1.87 (m, 2H), 1.86-1.71 (m, 3H), 1.60-1.47 (m, 1H).

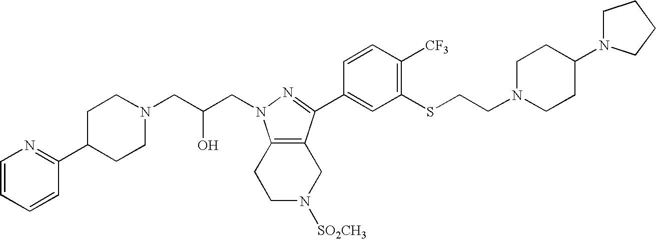

### Example 185

### 1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol

MS (ESI): mass calcd. for C₃₈H₅₂F₃N₇O₃S₂, 775.3; m/z found, 776.3 [M+H]⁺. ¹H NMR: 8.60 (d, J=5.1, 1H), 8.04 (t, J=7.2, 1H), 7.90 (s, 1H), 7.82 (d, J=8.3, 1H), 7.78 (d, J=8.2, 1H), 7.54 (d, J=7.9, 1H), 7.52-7.47 (m, 1H), 4.65-4.45 (m, 3H), 4.28 (dd, J=14.4, 4.0, 1H), 4.21 (dd, J=14.4, 6.5, 1H), 3.86-3.73 (m, 4H), 3.73-3.62 (m, 4H), 3.49 (dd, J=10.3, 5.3, 2H), 3.47-3.35 (m, 4H), 3.35-2.89 (m, 15H), 2.43 (d, J=13.5, 2H), 2.35-1.83 (m, 10H).

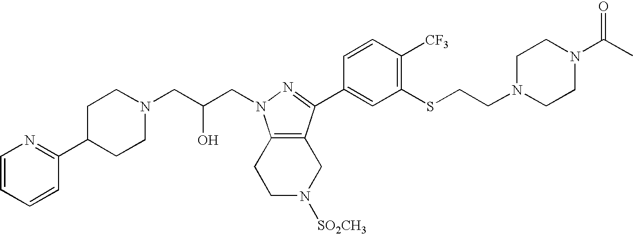

### Example 186

### 1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol

MS (ESI): mass calcd. for C₃₅H₄₆F₃N₇O₄S₂, 749.3; m/z found, 750.3 [M+H]⁺. ¹H NMR: 8.68 (d, J=5.3, 1H), 8.23 (t, J=6.6, 1H), 7.92 (s, 1H), 7.81 (d, J=8.3, 1H), 7.75-7.69 (m, 2H), 7.69-7.62 (m, 1H), 4.72-4.43 (m, 3H), 4.28 (dd, J=14.4, 3.6, 1H), 4.20 (dd, J=14.4, 6.8, 1H), 4.12-3.09 (m, 22H), 3.09-2.91 (m, 5H), 2.49-1.97 (m, 7H).

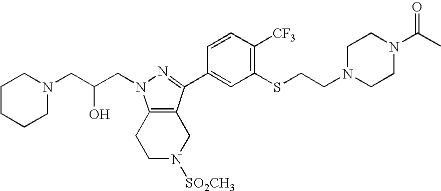

### Example 187

### 1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

MS (ESI): mass calcd. for C₃₀H₄₃F₃N₆O₄S₂, 672.2; m/z found, 673.2 [M+H]⁺. ¹H NMR: 7.93 (s, 1H), 7.83 (d, J=8.3, 1H), 7.74 (d, J=8.2, 1H), 4.64-4.55 (m, 2H), 4.50 (ddd, J=13.7, 6.7, 3.8, 1H), 4.26 (dd, J=14.4, 4.0, 1H), 4.18 (J=14.4, 6.8, 1H), 3.84 (s, 3H), 3.74-3.14 (m, 18H), 3.13-2.91 (m, 7H), 2.15 (s, 1H), 2.05-1.66 (m, 5H), 1.62-1.47 (m, 1H).

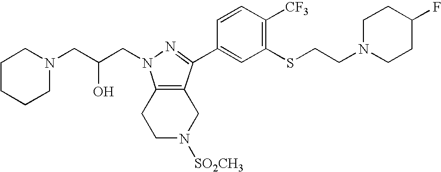

### Example 188

### 1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

MS (ESI): mass calcd. for C₂₉H₄₁F₄N₅O₃S₂, 647.2; m/z found, 648.2 [M+H]⁺. ¹H NMR: 7.93 (s, 1H), 7.81 (d, J=8.3, 1H), 7.71 (d, J=8.2, 1H), 4.61-4.53 (m, 2H), 4.52-4.46 (m, 1H), 4.24 (dd, J=14.5, 4.1, 1H), 4.17 (dd, J=14.4, 6.8, 1H), 3.80-2.85 (m, 23H), 2.39-1.41 (m, 10H).

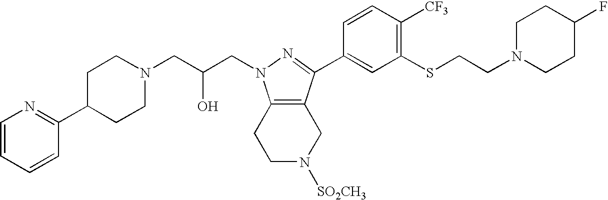

### Example 189

### 1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol

MS (ESI): mass calcd. for C₃₄H₄₄F₄N₆O₃S₂, 724.2; m/z found, 725.2 [M+H]⁺. ¹H NMR: 8.68-8.63 (m, 1H), 8.17 (t, J=7.3, 1H), 7.94 (s, 1H), 7.83 (d, J=8.3, 1H), 7.74 (d, J=8.2, 1H), 7.66 (d, J=8.0, 1H), 7.64-7.59 (m, 1H), 4.64-4.50 (m, 3H), 4.28 (dd, J=14.4, 3.9, 1H), 4.21 (dd, J=14.4, 6.6, 1H), 3.80 (t, J=14.1, 2H), 3.72-3.61 (m, 2H), 3.62-2.95 (m, 20H), 2.45-1.84 (m, 9H).

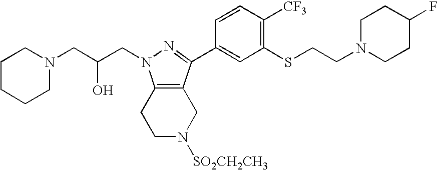

### Example 190

### 1-{5-(Ethylsulfonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol

The title compound was prepared using methods analogous to those described for Example 234. MS (ESI): mass calcd. for C₃₀H₄₃F₄N₅O₃S₂, 661.2; m/z found, 662.2 [M+H]⁺. ¹H NMR: 7.97-7.91 (m, 1H), 7.81 (d, J=8.2, 1H), 7.68 (d, J=8.1, 1H), 4.70-4.55 (m, 2H), 4.50 (ddd, J=13.7, 6.8, 4.0, 1H), 4.23 (dd, J=14.4, 4.1, 1H), 4.16 (dd, J=14.4, 6.8, 1H), 3.74-3.65 (m, 2H), 3.65-2.91 (m, 19H), 2.40-1.69 (m, 10H), 1.58-1.45 (m, 1H), 1.33 (t, J=7.4, 3H).

Examples 191-195 were prepared using methods similar to those described in Example 153, with the appropriate substituent changes.

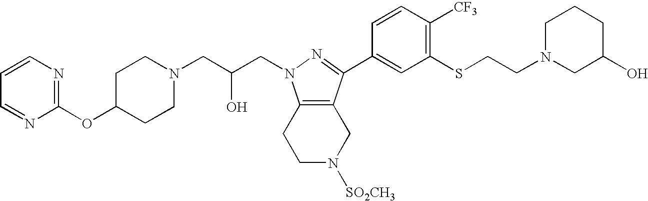

### Example 191

### 1-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-3-ol

MS (ESI): mass calcd. for C₃₃H₄₄F₃N₇O₅S₂, 739.2; m/z found, 740.2 [M+H]⁺. ¹H NMR: 8.70-8.55 (m, 2H), 7.95 (s, 1H), 7.82 (d, J=8.1, 1H), 7.79-7.64 (m, 1H), 7.14 (s, 1H), 4.67-4.46 (m, 3H), 4.32-4.12 (m, 3H), 3.90-2.60 (m, 23H), 2.55-1.92 (m, 6H), 1.84 (d, J=12.4, 1H), 1.75 (d, J=14.0, 1H), 1.66 (t, J=12.4, 1H).

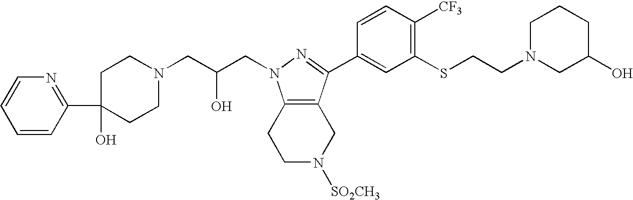

### Example 192

### 1-(2-{[5-{1-[2-Hydroxy-3-(4-hydroxy-4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-3-ol

MS (ESI): mass calcd. for C₃₄H₄₅F₃N₆O₅S₂, 738.2; m/z found, 739.2 [M+H]⁺. ¹H NMR: 8.63 (d, J=4.4, 1H), 8.13 (s, 1H), 8.02-7.93 (m, 1H), 7.86 (d, J=7.9, 1H), 7.82 (d, J=8.1, 1H), 7.78-7.67 (m, 1H), 7.57 (s, 1H), 4.74-4.45 (m, 3H), 4.38-4.09 (m, 3H), 4.00-3.24 (m, 18H), 3.23-2.95 (m, 7H), 2.69-2.48 (m, 2H), 2.20 (s, 1H), 2.05-1.95 (m, 1H), 1.85 (d, J=12.7, 1H), 1.76 (d, J=14.5, 1H), 1.71-1.61 (m, 1H).

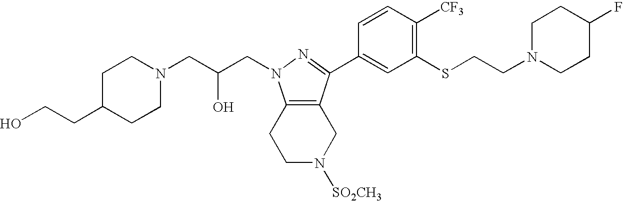

### Example 193

### 1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(2-hydroxyethyl)piperidin-1-yl]propan-2-ol

MS (ESI): mass calcd. for C₃₁H₄₅F₄N₅O₄S₂, 691.2; m/z found, 692.2 [M+H]⁺. ¹H NMR: 8.01 (s, 1H), 7.81 (s, 1H), 7.67 (s, 1H), 5.49 (s, 1H), 4.61 (s, 3H), 4.40-4.13 (m, 2H), 3.84-2.81 (m, 23H), 2.44-2.12 (m, 4H), 2.11-1.87 (m, 3H), 1.87-1.24 (m, 6H).

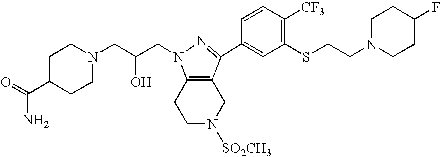

### Example 194

### 1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxamide

MS (ESI): mass calcd. for C₃₀H₄₂F₄N₆O₄S₂, 690.2; m/z found, 691.2 [M+H]⁺. ¹H NMR: 7.95 (s, 1H), 7.84 (d, J=8.3, 1H), 7.7 (d, J=8.2, 1H), 4.67-4.44 (m, 3H), 4.26 (dd, J=14.3, 4.1, 1H), 4.19 (dd, J=14.4, 6.6, 1H), 3.85-2.89 (m, 26H), 2.36-1.85 (m, 8H).

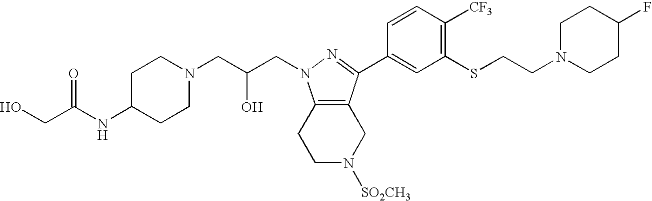

### Example 195

### N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-2-hydroxyacetamide

MS (ESI): mass calcd. for C₃₁H₄₄F₄N₆O₅S₂, 720.2; m/z found, 721.2 [M+H]⁺. ¹H NMR: 7.95 (s, 1H), 7.83 (d, J=8.3, 1H), 7.73 (d, J=8.1, 1H), 4.66-4.47 (m, 3H), 4.31-4.16 (m, 2H), 3.99 (s, 3H), 3.81-2.95 (m, 25H), 2.38-1.79 (m, 8H).

Examples 196-214 were prepared using methods analogous to those described in Example 140, substituting the appropriate amines and using (R)-(−)-glycidyl nosylate or (S)-(+)-glycidyl nosylate as needed.

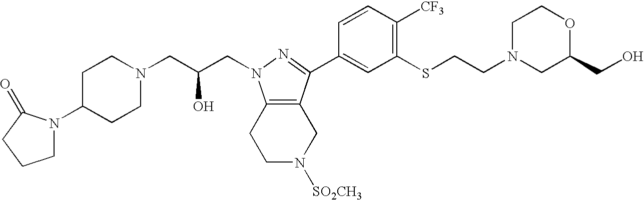

### Example 196

### 1-{1-[(2S)-2-Hydroxy-3-{3-[3-({2-[(2R)-2-(hydroxymethyl)morpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one

MS (ESI): mass calcd. for C₃₃H₄₇F₃N₆O₆S₂, 744.30; m/z found, 745.3 [M+H]⁺. ¹H NMR: 7.96 (s, 1H), 7.87-7.83 (m, 1H), 7.77-7.74 (m, 1H), 4.62-4.57 (m, 2H), 4.55-4.47 (m, 1H), 4.31-4.07 (m, 4H), 3.89-3.40 (m, 18H), 3.30-3.09 (m, 3H), 3.07-2.94 (m, 6H), 2.46-2.35 (m, 2H), 2.21-1.88 (m, 6H).

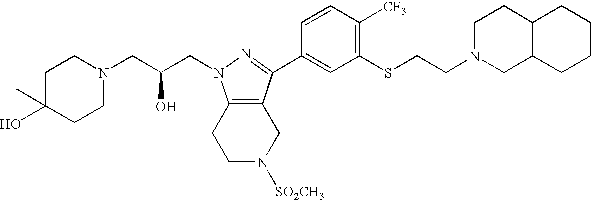

### Example 197

### 1-[(2S)-2-Hydroxy-3-[5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]-4-methylpiperidin-4-ol

MS (ESI): mass calcd. for C₃₄H₅₀F₃N₅O₄S₂, 713.33; m/z found, 714.3 [M+H]⁺. ¹H NMR: 7.97-7.91 (m, 1H), 7.82 (d, J=8.2, 1H), 7.72 (d, J=8.1, 1H), 4.61-4.47 (m, 3H), 4.24 (dd, J=14.4, 4.0, 1H), 4.17 (dd, J=14.4, 6.8, 1H), 3.72-3.54 (m, 3H), 3.53-3.45 (m, 3H), 3.44-3.31 (m, 6H), 3.28-3.18 (m, 3H), 3.06-2.92 (m, 6H), 2.08-1.87 (m, 2H), 1.87-1.56 (m, 9H), 1.54-1.29 (m, 5H), 1.29-1.15 (m, 4H), 1.08-0.96 (m, 1H).

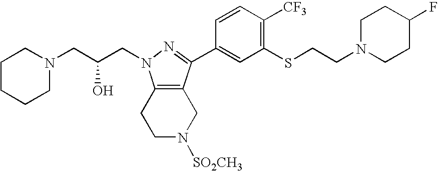

### Example 198

### (2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

MS (ESI): mass calcd. for C₂₉H₄₁F₄N₅O₃S₂, 647.2; m/z found, 648.2 [M+H]⁺. ¹H NMR: 7.93 (s, 1H), 7.81 (d, J=8.3, 1H), 7.71 (d, J=8.2, 1H), 4.63-4.53 (m, 2H), 4.53-4.46 (m, 1H), 4.23 (dd, J=14.4, 4.1, 1H), 4.17 (dd, J=14.4, 6.7, 1H), 3.80-3.23 (m, 15H), 3.18 (dd, J=13.3, 10.8, 1H), 3.13-2.90 (m, 7H), 2.44-1.65 (m, 9H), 1.62-1.42 (m, 1H).

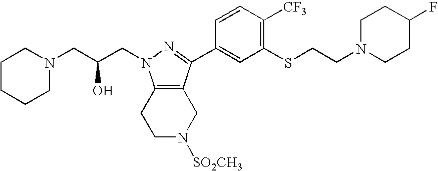

### Example 199

### (2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

MS (ESI): mass calcd. for C₂₉H₄₁F₄N₅O₃S₂, 647.2; m/z found, 648.2 [M+H]⁺. ¹H NMR: 7.93 (s, 1H), 7.81 (d, J=8.3, 1H), 7.70 (d, J=8.1, 1H), 4.60-4.53 (m, 2H), 4.52-4.46 (m, 1H), 4.23 (dd, J=14.4, 4.0, 1H), 4.17 (dd, J=14.4, 6.7, 1H), 3.75-3.24 (m, 16H), 3.18 (dd, J=13.3, 10.8, 1H), 3.11-2.90 (m, 7H), 2.35-1.66 (m, 9H), 1.56-1.47 (m, 1H).

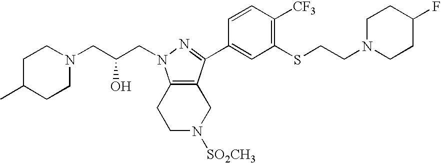

### Example 200

### (2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol

MS (ESI): mass calcd. for C₃₀H₄₃F₄N₅O₃S₂, 661.2; m/z found, 662.2 [M+H]⁺. ¹H NMR: 7.93 (s, 1H), 7.81 (d, J=8.2, 1H), 7.71 (d, J=8.1, 1H), 4.62-4.53 (m, 2H), 4.52-4.46 (m, 1H), 4.23 (dd, J=14.5, 4.1, 1H), 4.17 (dd, J=14.4, 6.7, 1H), 3.73-3.37 (m, 12H), 3.37-3.23 (m, 7H), 3.20 (dd, J=13.2, 10.7, 1H), 2.42-1.95 (m, 4H), 1.94-1.83 (m, 2H), 1.76-1.57 (m, 1H), 1.52 (ddd, J=16.0, 13.9, 4.0, 1H), 1.43 (ddd, J=15.9, 14.2, 4.0, 1H), 0.99 (d, J=6.5, 3H).

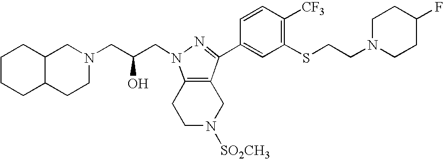

### Example 201

### (2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(octahydroisoquinolin-2(1H)-yl)propan-2-ol

MS (ESI): mass calcd. for C₃₃H₄₇F₄N₅O₃S₂, 701.3; m/z found, 702.3 [M+H]⁺. ¹H NMR: 7.93 (s, 1H), 7.83 (d, J=8.2, 1H), 7.74 (d, J=7.2, 1H), 4.73-4.41 (m, 3H), 4.29-4.21 (m, 1H), 4.18 (dd, J=14.4, 6.5, 1H), 3.83-2.95 (m, 24H), 2.61-0.87 (m, 15H).

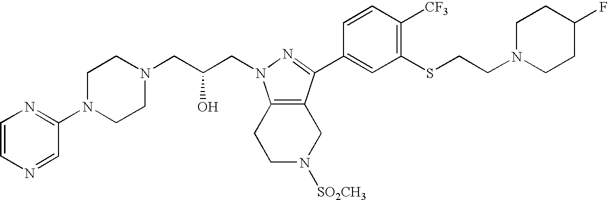

### Example 202

### (2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrazin-2-ylpiperazin-1-yl)propan-2-ol

MS (ESI): mass calcd. for C₃₂H₄₂F₄N₈O₃S₂, 726.2; m/z found, 727.2 [M+H]⁺. ¹H NMR: 8.32 (s, 1H), 8.18 (dd, J=2.6, 1.5, 1H), 7.94 (s, 1H), 7.93 (d, J=2.6, 1H), 7.82 (d, J=8.3, 1H), 7.73 (d, J=8.0, 1H), 4.67-4.51 (m, 3H), 4.27 (dd, J=14.5, 4.2, 1H), 4.21 (dd, J=14.5, 6.5, 1H), 3.78-3.19 (m, 21H), 3.08-2.94 (m, 6H), 2.46-1.79 (m, 4H).

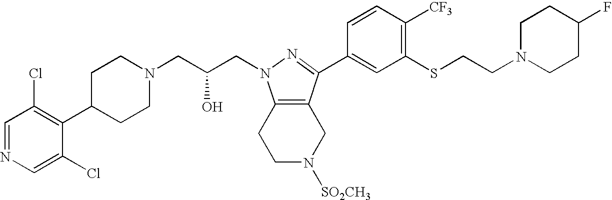

### Example 203

### (2R)-1-[4-(3,5-Dichloropyridin-4-yl)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol

MS (ESI): mass calcd. for C₃₄H₄₂Cl₂F₄N₆O₃S₂, 792.2; m/z found, 793.2 [M+H]⁺. ¹H NMR: 8.34 (s, 2H), 7.85 (s, 1H), 7.72 (d, J=8.3, 1H), 7.63 (d, J=8.1, 1H), 4.59-4.43 (m, 3H), 4.19 (dd, J=14.4, 4.2, 1H), 4.12 (dd, J=14.5, 6.5, 1H), 3.86-2.81 (m, 28H), 2.36-1.76 (m, 4H).

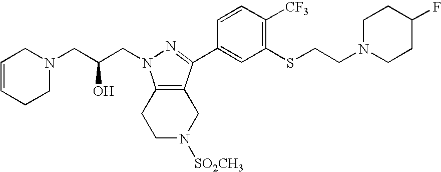

### Example 204

### (2S)-1-(3,6-Dihydropyridin-1(2H)-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol

MS (ESI): mass calcd. for C₂₉H₃₉F₄N₅O₃S₂, 645.2; m/z found, 646.2 [M+H]⁺. ¹H NMR: 7.93 (s, 1H), 7.82 (d, J=8.2, 1H), 7.72 (d, J=8.2, 1H), 5.99 (d, J=10.4, 1H), 5.74 (d, J=10.4, 1H), 4.62-4.47 (m, 3H), 4.26 (dd, J=14.4, 4.1, 1H), 4.19 (dd, J=14.5, 6.7, 1H), 4.01-3.08 (m, 18H), 3.07-2.94 (m, 5H), 2.69-1.81 (m, 6H).

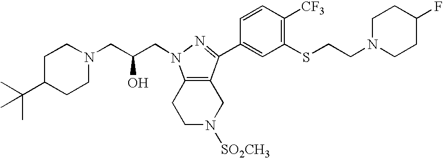

### Example 205

### (2S)-1-[4-(1,1-Dimethylethyl)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol

MS (ESI): mass calcd. for C₃₃H₄₉F₄N₅O₃S₂, 703.3; m/z found, 704.3 [M+H]⁺. ¹H NMR: 7.94 (s, 1H), 7.81 (d, J=8.3, 1H), 7.71 (d, J=8.1, 1H), 4.62-4.46 (m, 3H), 4.24 (dd, J=14.4, 4.1, 1H), 4.17 (dd, J=14.5, 6.6, 1H), 3.78-3.09 (m, 16H), 3.06-2.88 (m, 7H), 2.46-1.76 (m, 6H), 1.64 (dq, J=13.8, 4.0, 1H), 1.55 (dq, J=13.6, 3.9, 1H), 1.36 (tt, J=12.4, 2.9, 1H), 0.90 (s, 9H).

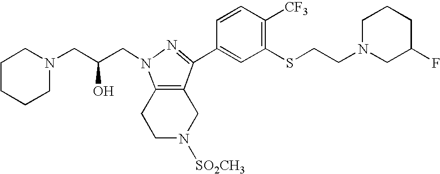

### Example 206

### (2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

MS (ESI): mass calcd. for C₂₉H₄₁F₄N₅O₃S₂, 647.2; m/z found, 648.2 [M+H]⁺. ¹H NMR: 7.84 (s, 1H), 7.71 (d, J=8.3, 1H), 7.61 (d, J=8.3, 1H), 4.51-4.44 (m, 2H), 4.40 (ddd, J=13.7, 6.8, 3.8, 1H), 4.14 (dd, J=14.4, 4.1, 1H), 4.07 (dd, J=14.5, 6.8, 1H), 3.81-2.79 (m, 23H), 2.04-1.94 (m, 2H), 1.88-1.59 (m, 7H), 1.49-1.36 (m, 1H).

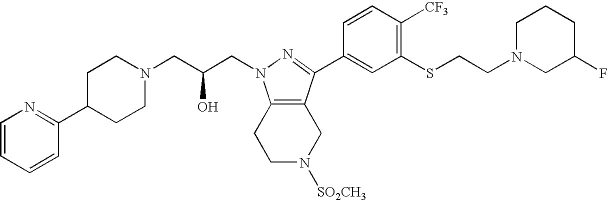

### Example 207

### (2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol

MS (ESI): mass calcd. for C₃₄H₄₄F₄N₆O₃S₂, 724.2; m/z found, 725.2 [M+H]⁺. ¹H NMR: 8.71 (d, J=5.4, 1H), 8.32 (t, J=6.5, 1H), 7.95 (s, 1H), 7.83-7.70 (m, 4H), 4.65-4.48 (m, 2H), 4.28 (dd, J=14.4, 3.8, 1H), 4.21 (dd, J=14.4, 6.7, 1H), 3.90-2.93 (m, 26H), 2.41-1.64 (m, 8H).

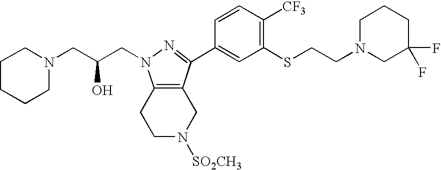

### Example 208

### (2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

MS (ESI): mass calcd. for C₂₉H₄₀F₅N₅O₃S₂, 665.2; m/z found, 666.2 [M+H]⁺. ¹H NMR: 7.96 (s, 1H), 7.82 (d, J=8.2, 1H), 7.69 (d, J=8.1, 1H), 4.79-4.47 (m, 3H), 4.25 (dd, J=14.4, 4.1, 1H), 4.18 (dd, J=14.5, 6.8, 1H), 3.98-2.89 (m, 22H), 2.40-1.48 (m, 10H).

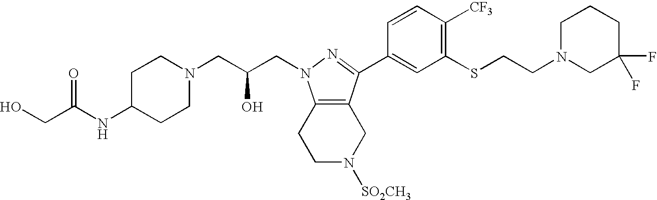

### Example 209

### N-{1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide

MS (ESI): mass calcd. for C₃₁H₄₃F₅N₆O₅S₂, 738.2; m/z found, 739.2 [M+H]⁺. ¹H NMR: 7.94 (s, 1H), 7.81 (d, J=8.2, 1H), 7.69 (d, J=7.8, 1H), 4.71-4.45 (m, 3H), 4.31-4.16 (m, 2H), 3.97 (s, 3H), 3.89-2.93 (m, 23H), 2.26-1.77 (m, 8H).

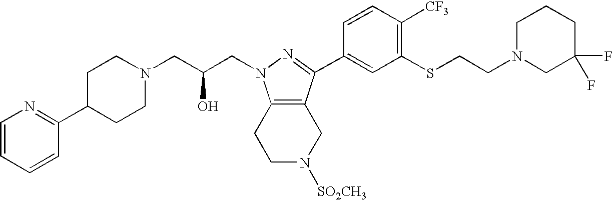

### Example 210

### (2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol

MS (ESI): mass calcd. for C₃₄H₄₃F₅N₆O₃S₂, 742.2; m/z found, 743.3 [M+H]⁺. ¹H NMR: 8.57 (d, J=4.4, 1H), 8.06-7.92 (m, 2H), 7.82 (d, J=8.1, 1H), 7.72 (d, J=8.0, 1H), 7.49 (d, J=7.8, 1H), 7.47-7.42 (m, 1H), 4.69-4.49 (m, 3H), 4.28 (dd, J=14.2, 3.9, 1H), 4.21 (dd, J=14.4, 6.7, 1H), 4.00-2.93 (m, 22H), 2.38-1.94 (m, 9H).

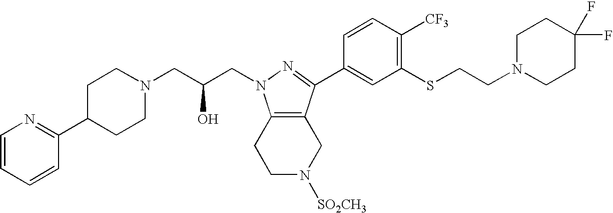

### Example 211

### (2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

MS (ESI): mass calcd. for C₂₉H₄₀F₅N₅O₃S₂, 665.3; m/z found, 666.3 [M+H]⁺. ¹H NMR: 7.93 (s, 1H), 7.81 (d, J=8.3, 1H), 7.72 (d, J=8.0, 1H), 4.63-4.54 (m, 2H), 4.49 (ddd, J=13.6, 6.8, 3.9, 1H), 4.24 (dd, J=14.4, 4.1, 1H), 4.17 (dd, J=14.4, 6.7, 1H), 3.74-3.28 (m, 14H), 3.18 (dd, J=13.3, 10.8, 1H), 3.10-2.94 (m, 7H), 2.41-2.29 (m, 4H), 1.97-1.70 (m, 5H), 1.58-1.48 (m, 1H).

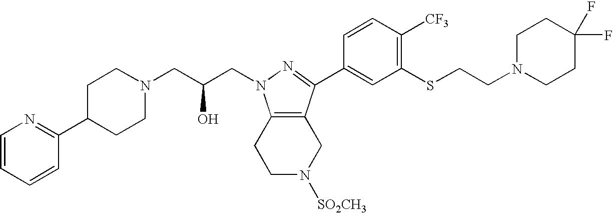

### Example 212

### (2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol

MS (ESI): mass calcd. for C₃₄H₄₃F₅N₆O₃S₂, 742.3; m/z found, 743.3 [M+H]⁺. ¹H NMR: 8.70 (dd, J=5.4, 0.9, 1H), 8.28 (t, J=7.1, 1H), 7.94 (s, 1H), 7.82 (d, J=8.3, 1H), 7.78-7.67 (m, 3H), 4.62-4.49 (m, 3H), 4.28 (dd, J=14.4, 4.0, 1H), 4.21 (dd, J=14.4, 6.6, 1H), 3.88-3.18 (m, 18H), 3.06-2.95 (m, 5H), 2.41-2.09 (m, 8H).

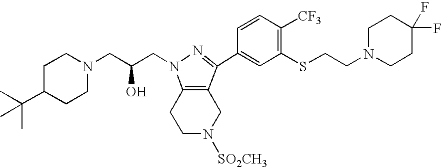

### Example 213

### (2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1,1-dimethylethyl)piperidin-1-yl]propan-2-ol

MS (ESI): mass calcd. for C₃₃H₄₈F₅N₅O₃S₂, 721.3; m/z found, 722.3 [M+H]⁺. ¹H NMR: 7.93 (s, 1H), 7.82 (d, J=8.3, 1H), 7.73 (d, J=8.2, 1H), 4.57 (d, J=3.5, 2H), 4.52-4.47 (m, 1H), 3.73-3.35 (m, 10H), 3.31 (td, J=3.3, 1.6, 1.6, 5H), 3.26 (dd, J=13.2, 2.9, 1H), 3.20 (dd, J=13.2, 10.6, 1H), 3.06-2.88 (m, 7H), 2.40-2.30 (m, 4H), 2.00-1.90 (m, 2H), 1.64 (ddd, J=17.0, 13.5, 4.0, 1H), 1.54 (dq, J=13.7, 3.9, 1H), 1.36 (tt, J=12.0, 2.8, 1H), 0.95-0.83 (m, 9H).

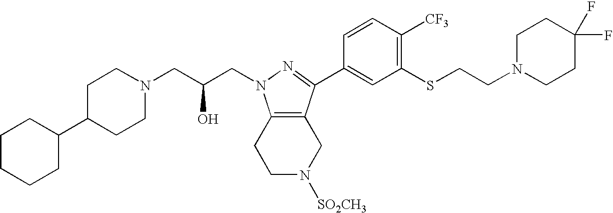

### Example 214

### (2S)-1-(4-Cyclohexylpiperidin-1-yl)-3-{3-[3-{[2-(4,4-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol

MS (ESI): mass calcd. for C₃₅H₅₀F₅N₅O₃S₂, 747.3; m/z found, 748.3 [M+H]⁺. ¹H NMR: 7.93 (s, 1H), 7.82 (d, J=8.2, 1H), 7.72 (d, J=8.1, 1H), 4.57 (d, J=4.0, 2H), 4.51-4.44 (m, 1H), 4.24 (dd, J=14.5, 4.1, 1H), 4.18 (dd, J=14.4, 6.5, 1H), 3.72-2.81 (m, 23H), 2.41-2.27 (m, 4H), 2.03-1.90 (m, 2H), 1.87-0.87 (m, 13H).

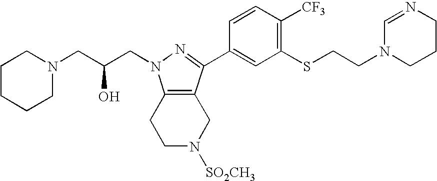

### Example 215

### (2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

A. 2-[5-(5-Methanesulfonyl-4,5,6,7-tetrahydro-1H-pyrazolo[4,3-c]pyridin-3-yl)-2-trifluoromethyl-phenylsulfanyl]-ethanol. This compound was prepared as described in Example 1, Step C. MS (ESI): mass calcd. for C₁₆H₁₈F₃N₃O₃S₂, 421.07; m/z found, 422.1 [M+H]⁺.

B. (2S)-1-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-3-piperidin-1-yl-propan-2-ol. This compound was prepared using methods similar to those described in Example 153, Step C. MS (ESI): mass calcd. for C₂₄H₃₃F₃N₄O₄S₂, 562.2; m/z found, 563.2 [M+H]⁺.

C. (2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl]-3-piperidin-1-ylpropan-2-ol. This compound was prepared using methods similar to those described in Example 4, Step A, with the appropriate substituent changes and replacing MsCl with p-toluenesulfonic acid (1.01 equiv.) and pyridine with both 4-(dimethylamino)-pyridine (0.32 equiv.) and TEA (0.14 equiv.). MS (ESI): mass calcd. for C₃₁H₃₉F₃N₄O₆S₃, 716.2; m/z found, 717.2 [M+H]⁺.

D. (2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl]-3-piperidin-1-ylpropan-2-ol. To a stirring solution of (2S)-1-{3-[3-{[2-(5,6-dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol (0.060 mmol) in 20% DCE/EtOH (v/v, 0.2 M) was added 1,4,5,6-tetrahydro-pyrimidine (5.0 eq) and TEA (10 eq). The reaction vessel was sealed and heated at 80° C. for 36 h. After cooling to rt the final compound was isolated via preparative HPLC. MS (ESI): mass calcd. for C₂₈H₃₉F₃N₆O₃S₂, 628.3; m/z found, 629.2 [M+H]⁺. ¹H NMR: 7.95 (d, J=18.8, 1H), 7.81 (d, J=8.2, 1H), 7.66 (d, J=8.0, 1H), 4.62-4.46 (m, 3H), 4.24 (dd, J=14.4, 4.1, 1H), 4.17 (dd, J=14.4, 6.8, 1H), 3.82-2.91 (m, 23H), 2.08-1.71 (m, 8H), 1.58-1.48 (m, 1H).

Examples 216-227 were prepared using methods similar to those described in Example 215, with the appropriate substituent changes.

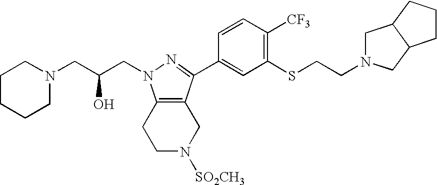

### Example 216

### (2S)-1-{3-[3-{[2-(Hexahydrocyclopenta[c]pyrrol-2(1H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

MS (ESI): mass calcd. for C₃₁H₄₄F₃N₅O₃S₂, 655.2; m/z found, 656.2 [M+H]⁺. ¹H NMR: 7.93 (s, 1H), 7.82 (d, J=8.3, 1H), 7.73 (d, J=8.2, 1H), 4.63-4.45 (m, 3H), 4.24 (dd, J=14.4, 4.1, 1H), 4.17 (dd, J=14.4, 6.7, 1H), 3.96-3.85 (m, 1H), 3.71-2.81 (m, 21H), 2.70-2.57 (m, 1H), 2.02-1.43 (m, 13H).

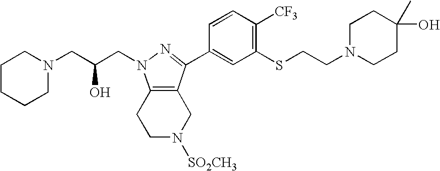

### Example 217

### 1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-methylpiperidin-4-ol

MS (ESI): mass calcd. for C₃₀H₄₄F₃N₅O₄S₂, 659.2; m/z found, 660.2 [M+H]⁺. ¹H NMR: 7.94 (s, 1H), 7.82 (d, J=8.3, 1H), 7.72 (d, J=7.9, 1H), 4.62-4.54 (m, 2H), 4.52-4.47 (m, 1H), 4.24 (dd, J=14.4, 4.1, 1H), 4.17 (dd, J=14.4, 6.7, 1H), 3.71-2.84 (m, 27H), 1.97-1.65 (m, 9H).

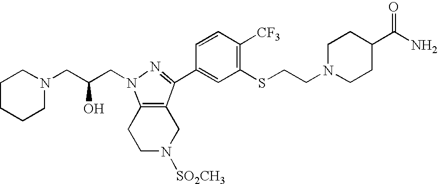

### Example 218

### 1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidine-4-carboxamide

MS (ESI): mass calcd. for C₃₀H₄₃F₃N₆O₄S₂, 672.2; m/z found, 673.2 [M+H]⁺. ¹H NMR: 7.94 (s, 1H), 7.83 (d, J=8.3, 1H), 7.73 (d, J=8.0, 1H), 4.61-4.54 (m, 2H), 4.52-4.47 (m, 1H), 4.24 (dd, J=14.4, 4.0, 1H), 4.17 (dd, J=14.4, 6.8, 1H), 3.73-2.88 (m, 26H), 2.13-1.73 (m, 9H).

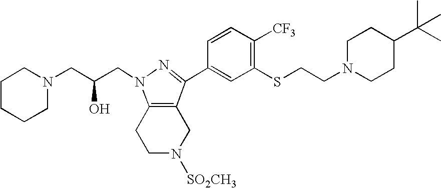

### Example 219

### (2S)-1-{3-[3-({2-[4-(1,1-Dimethylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol

MS (ESI): mass calcd. for C₃₃H₅₀F₃N₅O₃S₂, 685.3; m/z found, 686.3 [M+H]⁺. ¹H NMR: 7.93 (s, 1H), 7.83 (d, J=8.2, 1H), 7.74 (d, J=7.8, 1H), 4.61-4.55 (m, 2H), 4.53-4.47 (m, 1H), 4.24 (dd, J=14.4, 4.1, 1H), 4.17 (dd, J=14.4, 6.7, 1H), 3.74-2.88 (m, 33H), 2.04-1.26 (m, 9H).

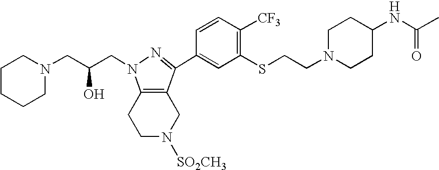

### Example 220

### N-[1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-yl]acetamide

MS (ESI): mass calcd. for C₃₁H₄₅F₃N₆O₄S₂, 686.3; m/z found, 687.3 [M+H]⁺. ¹H NMR: 7.93 (s, 1H), 7.83 (d, J=8.2, 1H), 7.74 (d, J=7.8, 1H), 4.62-4.46 (m, 3H), 4.24 (dd, J=14.4, 4.1, 1H), 4.17 (dd, J=14.4, 6.7, 1H), 3.76-2.87 (m, 27H), 2.04-1.19 (m, 10H).

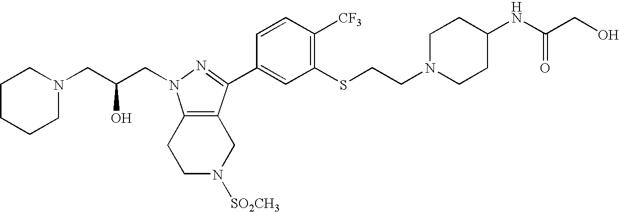

### Example 221

### 2-Hydroxy-N-[1-(2-{[5-{1-[(2S)-2-hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-yl]acetamide

MS (ESI): mass calcd. for C₃₁H₄₅F₃N₆O₅S₂, 702.3; m/z found, 703.3 [M+H]⁺. ¹H NMR: 7.93 (s, 1H), 7.83 (d, J=8.3, 1H), 7.73 (d, J=8.0, 1H), 4.62-4.47 (m, 3H), 4.24 (dd, J=14.3, 3.9, 1H), 4.17 (dd, J=14.4, 7.1, 1H), 3.97 (s, 3H), 3.72-2.94 (m, 24H), 2.19-1.47 (m, 10H).

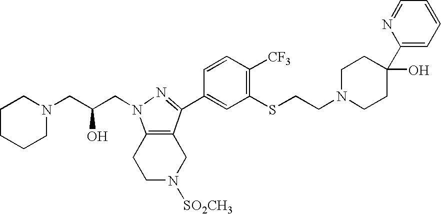

### Example 222

### 1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-pyridin-2-ylpiperidin-4-ol

MS (ESI): mass calcd. for C₃₄H₄₅F₃N₆O₄S₂, 722.3; m/z found, 723.2 [M+H]⁺. ¹H NMR: 8.64 (d, J=5.5, 1H), 8.25-8.15 (m, 1H), 7.97 (s, 1H), 7.91 (d, J=8.1, 1H), 7.83 (d, J=8.3, 1H), 7.72 (d, J=8.2, 1H), 7.65-7.60 (m, 1H), 4.62-4.54 (m, 2H), 4.53-4.48 (m, 1H), 4.24 (dd, J=14.5, 4.1, 1H), 4.17 (dd, J=14.4, 6.7, 1H), 3.76-2.92 (m, 24H), 2.56 (dt, J=14.5, 4.1, 2H), 2.06-1.44 (m, 8H).

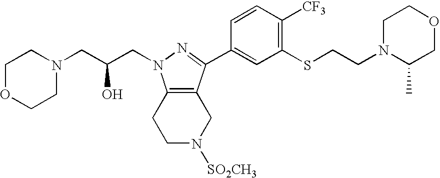

### Example 223

### (2S)-1-{3-[3-({2-[(3S)-3-Methylmorpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol

MS (ESI): mass calcd. for C₂₈H₄₀F₃N₅O₅S₂, 647.2; m/z found, 648.2 [M+H]⁺. ¹H NMR: 7.87 (s, 1H), 7.73 (d, J=8.2, 1H), 7.63 (d, J=8.0, 1H), 4.52-4.40 (m, 3H), 4.15 (dd, J=14.4, 4.1, 1H), 4.09 (dd, J=14.5, 6.6, 1H), 4.04-2.85 (m, 29H), 1.19 (d, J=1.4, 3H).

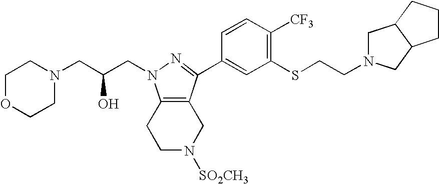

### Example 224

### (2S)-1-{3-[3-{[2-(Hexahydrocyclopenta[c]pyrrol-2(1H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol

MS (ESI): mass calcd. for C₃₀H₄₂F₃N₅O₄S₂, 657.3; m/z found, 658.3 [M+H]⁺. ¹H NMR: 7.89-7.78 (m, 1H), 7.72 (d, J=8.3, 1H), 7.65-7.62 (m, 1H), 4.52-4.39 (m, 3H), 4.16 (dd, J=14.5, 4.1, 1H), 4.09 (dd, J=14.5, 6.6, 1H), 4.02-2.70 (m, 29H), 2.55 (s, 1H), 1.98-1.12 (m, 5H).

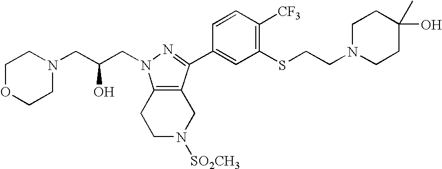

### Example 225

### 1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-methylpiperidin-4-ol

MS (ESI): mass calcd. for C₂₉H₄₂F₃N₅O₅S₂, 661.2; m/z found, 662.5 [M+H]⁺. ¹H NMR: 7.96 (s, 1H), 7.84 (d, J=8.2, 1H), 7.75 (d, J=8.5, 1H), 4.64-4.51 (m, 3H), 4.30-4.19 (m, 2H), 4.16-2.93 (m, 28H), 1.83 (d, J=1.7, 3H), 1.30 (s, 3H).

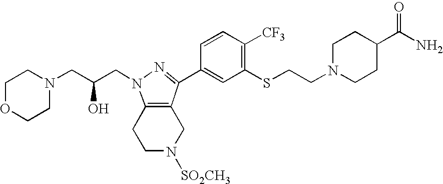

### Example 226

### 1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidine-4-carboxamide

MS (ESI): mass calcd. for C₂₉H₄₁F₃N₆O₅S₂, 674.3; m/z found, 675.3 [M+H]⁺. ¹H NMR: 7.84 (s, 1H), 7.73 (d, J=8.3, 1H), 7.63 (d, J=7.4, 1H), 4.52-4.41 (m, 3H), 4.16 (dd, J=14.4, 4.1, 1H), 4.09 (dd, J=14.5, 6.7, 1H), 4.03-2.84 (m, 28H), 2.43 (s, 1H), 2.11-1.75 (m, 4H).

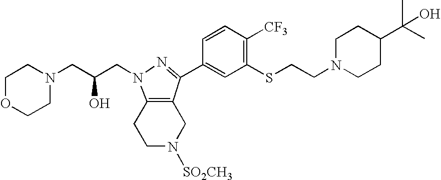

### Example 227

### (2S)-1-{3-[3-({2-[4-(1-Hydroxy-1-methylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol

MS (ESI): mass calcd. for C₃₁H₄₆F₃N₅O₅S₂, 689.3; m/z found, 690.3 [M+H]⁺. ¹H NMR: 7.84 (s, 1H), 7.72 (d, J=8.2, 1H), 7.63 (d, J=8.1, 1H), 4.54-4.40 (m, 3H), 4.16 (dd, J=14.4, 4.2, 1H), 4.09 (dd, J=14.5, 6.6, 1H), 4.01-2.80 (m, 28H), 1.94 (d, J=10.8, 1H), 1.56-1.45 (m, 3H), 1.07 (s, 6H).

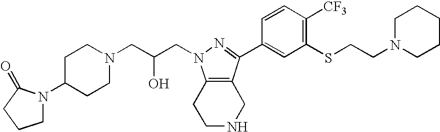

### Example 228

### 1-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one

A. 1-{2-Hydroxy-3-[4-(2-oxo-pyrrolidin-1-yl)-piperidin-1-yl]-propyl}-3-[3-(2-piperidin-1-yl-ethylsulfanyl)-4-trifluoromethyl-phenyl]-1,4,6,7-tetrahydro-pyrazolo[4,3-c]pyridine-5-carboxylic acid tert-butyl ester. This compound was prepared from 3-(3-nitro-4-trifluoromethyl-phenyl)-1,4,6,7-tetrahydro-pyrazolo[4,3-c]pyridine-5-carboxylic acid tert-butyl ester using methods similar to those described in Example 153, Steps A-C, substituting 1-piperidin-4-yl-pyrrolidin-2-one for piperidine. MS (ESI): mass calcd. for C₃₇H₅₃F₃N₆O₄S, 734.38; m/z found, 735.3 [M+H]⁺. ¹H NMR: 7.88-7.79 (m, 1H), 7.70-7.59 (m, 1H), 7.46 (s, 1H), 4.74-4.57 (m, 2H), 4.19-4.08 (m, 2H), 4.00 (dd, J=13.3, 6.7, 2H), 3.88-3.64 (m, 3H), 3.34 (t, J=7.0, 2H), 3.18-3.14 (m, 2H), 3.00 (d, J=11.4, 1H), 2.88 (d, J=10.6, 2H), 2.82-2.71 (m, 1H), 2.68-2.60 (m, 2H), 2.49-2.33 (m, 9H), 2.17-2.07 (m, 1H), 2.04-1.98 (m, 2H), 1.78-1.52 (m, 17H), 1.46-1.38 (m, 2H).

B. To a solution of 1-{2-hydroxy-3-[4-(2-oxo-pyrrolidin-1-yl)-piperidin-1-yl]-propyl}-3-[3-(2-piperidin-1-yl-ethylsulfanyl)-4-trifluoromethyl-phenyl]-1,4,6,7-tetrahydro-pyrazolo[4,3-c]pyridine-5-carboxylic acid tert-butyl ester (0.74 mmol) in CH₂Cl₂ (10 mL) was added HCl (4 M in 1,4-dioxane; 10 mL). The mixture was stirred for 1.5 h, concentrated, and the residue was suspended in MeOH. Dowex 550A basic resin (300 mg) was added and the mixture was stirred for 1 h. Filtration and concentration afforded a quantitative yield of the title compound. MS (ESI): mass calcd. for C₃₂H₄₅F₃N₆O₂S, 634.33; m/z found, 635.3 [M+H]⁺. ¹H NMR: 8.02 (s, 1H), 7.82 (d, J=8.2, 1H), 7.64 (d, J=8.2, 1H), 4.64-4.53 (m, 3H), 4.29 (dd, J=14.5, 4.4, 1H), 4.24 (dd, J=14.4, 6.6, 1H), 4.19-4.09 (m, 1H), 3.76 (t, J=11.0, 2H), 3.67-3.64 (m, 6H), 3.63-3.53 (m, 6H), 3.46-3.40 (m, 2H), 3.39-3.35 (m, 1H), 3.24-3.16 (m, 3H), 2.98 (dt, J=12.4, 3.0, 2H), 2.39 (t, J=8.1, 2H), 2.23-2.12 (m, 1H), 2.12-2.00 (m, 3H), 1.98-1.77 (m, 7H), 1.56-1.43 (m, 1H).

Examples 229-232 were prepared using methods similar to those described in Example 228, with the appropriate substituent changes.

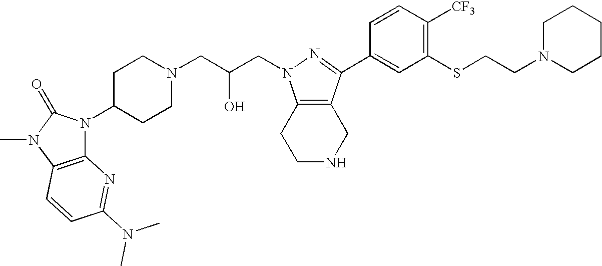

### Example 229

### 5-(Dimethylamino)-3-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-1-methyl-1,3-dihydro-2H-imidazo[4,5-b]pyridin-2-one

MS (ESI): mass calcd. for C₃₇H₅₀F₃N₉O₂S, 741.38; m/z found, 742.3 [M+H]⁺. ¹H NMR: 8.02 (s, 1H), 7.82 (d, J=8.2, 1H), 7.65 (dd, J=12.0, 8.1, 2H), 7.35 (d, J=8.2, 1H), 4.83-4.68 (m, 2H), 4.63-4.55 (m, 2H), 4.37-4.23 (m, 2H), 3.97-3.80 (m, 2H), 3.66-3.64 (m, 9H), 3.63-3.55 (m, 5H), 3.48-3.41 (m, 4H), 3.40-3.32 (m, 6H), 3.29-3.21 (m, 2H), 3.20-3.06 (m, 2H), 3.07-2.89 (m, 2H), 2.11 (t, J=10.9, 2H), 1.96-1.76 (m, 5H).

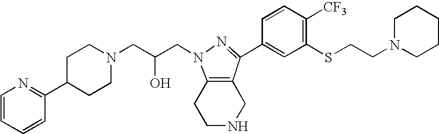

### Example 230

### 1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol

MS (ESI): mass calcd. for C₃₃H₄₃F₃N₆OS, 628.32; m/z found, 629.3 [M+H]⁺. ¹H NMR: 8.82 (d, J=5.6, 1H), 8.65 (t, J=7.8, 1H), 8.10 (d, J=8.1, 1H), 8.06-7.95 (m, 2H), 7.82 (d, J=8.1, 1H), 7.66 (d, J=7.8, 1H), 4.74-4.66 (m, 1H), 4.59 (s, 2H), 4.36-4.25 (m, 2H), 3.95-3.84 (m, 2H), 3.71-3.35 (m, 12H), 3.32-3.28 (m, 3H), 3.28-3.23 (m, 2H), 3.06-2.94 (m, 2H), 2.43-2.19 (m, 4H), 1.98-1.77 (m, 5H), 1.58-1.45 (m, 1H).

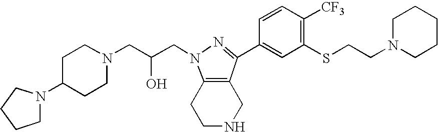

### Example 231

### 1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyrrolidin-1-ylpiperidin-1-yl)propan-2-ol

MS (ESI): mass calcd. for C₃₂H₄₇F₃N₆OS, 620.35; m/z found, 621.3 [M+H]⁺. ¹H NMR: 8.02 (s, 1H), 7.83 (d, J=8.2, 1H), 7.66 (d, J=8.0, 1H), 4.69-4.55 (m, 3H), 4.36-4.22 (m, 2H), 3.86 (d, J=11.8, 2H), 3.78-3.68 (m, 4H), 3.66-3.56 (m, 9H), 3.44 (d, J=12.9, 1H), 3.28-3.14 (m, 6H), 3.07-2.92 (m, 2H), 2.46 (t, J=14.3, 2H), 2.38-2.12 (m, 5H), 2.11-2.00 (m, 2H), 1.98-1.78 (m, 5H), 1.59-1.47 (m, 1H).

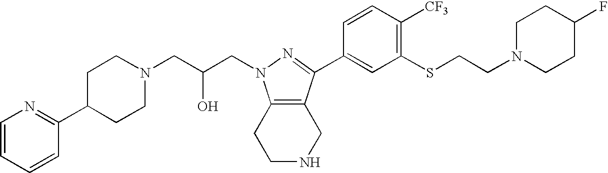

### Example 232

### 1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol

MS (ESI): mass calcd. for C₃₃H₄₂F₄N₆OS, 646.3; m/z found, 647.3 [M+H]⁺. ¹H NMR: 8.69 (d, J=5.4, 1H), 8.25 (s, 1H), 7.99 (s, 1H), 7.83 (d, J=8.2, 1H), 7.74 (dd, J=7.7, 3.9, 1H), 7.69 (s, 1H), 7.65 (d, J=8.2, 1H), 4.59 (s, 1H), 4.56-4.49 (m, 2H), 4.27 (ddd, J=21.4, 14.4, 5.3, 2H), 3.83 (dd, J=16.2, 14.9, 2H), 3.61 (t, J=6.3, 2H), 3.59-3.27 (m, 17H), 3.22 (dd, J=12.0, 5.9, 2H), 2.44-1.86 (m, 8H).

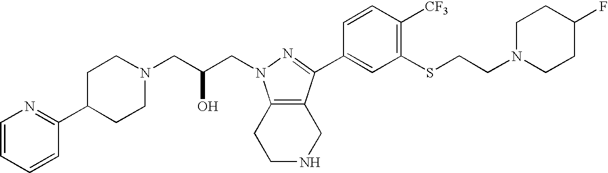

### Example 233

### (2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol

This compound was prepared using methods analogous to those described in Example 228, substituting (S)-(+)-glycidyl nosylate for epichlorohydrin. MS (ESI): mass calcd. for C₃₃H₄₂F₄N₆OS, 646.31; m/z found, 647.3 [M+H]⁺. ¹H NMR: 8.80 (d, J=5.5, 1H), 8.52 (t, J=7.9, 1H), 8.00 (s, 1H), 7.96 (d, J=7.5, 1H), 7.93-7.90 (m, 1H), 7.82 (d, J=8.3, 1H), 7.65 (d, J=8.2, 1H), 4.97-4.89 (m, 1H), 4.65-4.57 (m, 1H), 4.53 (s, 2H), 4.31 (dd, J=14.4, 3.4, 1H), 4.23 (dd, J=14.4, 6.9, 1H), 3.86 (t, J=11.3, 2H), 3.61 (t, J=6.3, 3H), 3.58-3.49 (m, 4H), 3.48-3.36 (m, 5H), 3.36-3.32 (m, 2H), 3.29-3.17 (m, 5H), 2.39-1.96 (m, 8H).

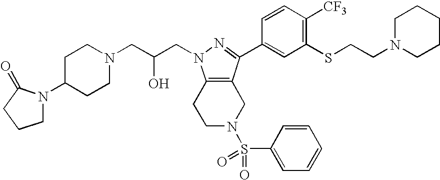

### Example 234

### 1-(1-{2-Hydroxy-3-[5-(phenylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one

To a solution of benzenesulfonyl chloride (0.011 mL, 0.087 mmol) in CH₂Cl₂ (1 mL) was added 1-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one (50 mg, 0.079 mmol). The mixture was stirred for 15 h, concentrated, dissolved in DMF, and purified using reverse phase HPLC. MS (ESI): mass calcd. for C₃₈H₄₉F₃N₆O₄S₂, 774.32; m/z found, 775.3 [M+H]⁺. ¹H NMR: 8.10-8.01 (m, 1H), 7.89 (d, J=6.2, 2H), 7.86-7.81 (m, 1H), 7.71-7.65 (m, 1H), 7.65-7.55 (m, 3H), 4.59 (s, 1H), 4.52-4.40 (m, 2H), 4.30-4.06 (m, 3H), 3.75 (s, 2H), 3.65-3.50 (m, 6H), 3.44 (s, 2H), 3.40-3.31 (m, 3H), 3.29-3.10 (m, 3H), 3.10-2.81 (m, 4H), 2.40 (t, J=7.0, 2H), 2.26-1.99 (m, 4H), 1.98-1.76 (m, 8H), 1.56-1.45 (m, 1H).

Examples 235-237 were prepared using methods similar to those described in Example 234, with the appropriate substituent changes.

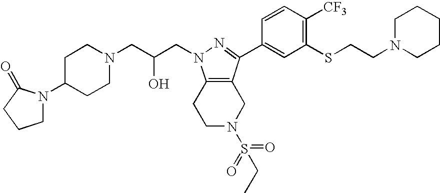

### Example 235

### 1-(1-{3-[5-(Ethylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-2-hydroxypropyl}piperidin-4-yl)pyrrolidin-2-one

MS (ESI): mass calcd. for C₃₄H₄₉F₃N₆O₄S₂, 726.32; m/z found, 727.3 [M+H]⁺. ¹H NMR: 8.08-8.04 (m, 1H), 7.84 (s, 1H), 7.72-7.62 (m, 1H), 4.66 (s, 3H), 4.33 (d, J=40.1, 2H), 4.24-4.10 (m, 1H), 3.84-3.69 (m, 4H), 3.68-3.54 (m, 5H), 3.51-3.43 (m, 3H), 3.43-3.32 (m, 3H), 3.26-3.13 (m, 3H), 3.09-2.92 (m, 4H), 2.47-2.38 (m, 2H), 2.31-2.09 (m, 2H), 2.10-2.00 (m, 2H), 2.01-1.75 (m, 7H), 1.57-1.45 (m, 1H), 1.38-1.30 (m, 4H).

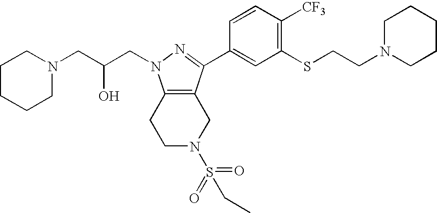

### Example 236

### 1-[5-(Ethylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol

MS (ESI): mass calcd. for C₃₀H₄₄F₃N₅O₃S₂, 643.28; m/z found, 644.3 [M+H]⁺. ¹H NMR: 8.06-7.90 (m, 1H), 7.81 (d, J=8.2, 1H), 7.67 (d, J=8.1, 1H), 4.65-4.60 (m, 2H), 4.59-4.52 (m, 1H), 4.25 (dd, J=14.4, 3.9, 1H), 4.18 (dd, J=14.3, 6.7, 1H), 3.74-3.67 (m, 2H), 3.64-3.51 (m, 6H), 3.38-3.32 (m, 4H), 3.18 (dd, J=14.6, 7.3, 3H), 3.09 (t, J=10.0, 1H), 3.04-2.93 (m, 5H), 2.00-1.72 (m, 10H), 1.58-1.45 (m, 2H), 1.34 (t, J=7.3, 3H).

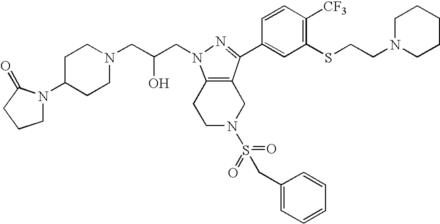

### Example 237

### 1-{1-[2-Hydroxy-3-(5-[(phenylmethyl)sulfonyl]-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one

MS (ESI): mass calcd. for C₃₉H₅₁F₃N₆O₄S₂, 788.34; m/z found, 789.3 [M+H]⁺. ¹H NMR: 7.96 (s, 1H), 7.81 (d, J=7.3, 1H), 7.59 (d, J=6.4, 1H), 7.48-7.40 (m, 2H), 7.37-7.33 (m, 3H), 4.62 (s, 1H), 4.53-4.44 (m, 4H), 4.34-4.10 (m, 3H), 3.80-3.70 (m, 2H), 3.64-3.51 (m, 7H), 3.46-3.40 (m, 2H), 3.40-3.31 (m, 3H), 3.29-3.14 (m, 2H), 3.04-2.93 (m, 2H), 2.87-2.81 (m, 2H), 2.41 (t, J=7.6, 2H), 2.25-2.15 (m, 1H), 2.16-2.08 (m, 1H), 2.08-2.00 (m, 2H), 1.99-1.76 (m, 7H), 1.56-1.42 (m, 1H).

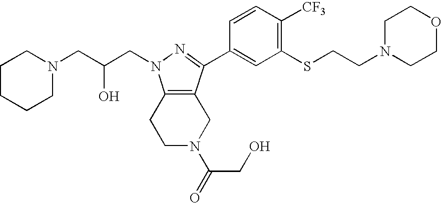

### Example 238

### 1-[5-(Hydroxyacetyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol

A mixture of 1-(3-{3-[(2-morpholin-4-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol (50 mg, 0.09 mmol) (prepared analogously to Example 228), glycolic acid (27 mg, 0.36 mmol), HATU (69 mg, 0.18 mmol), HOAt (0.5 M in DMF; 0.35 mL), and DIPEA (0.05 mL, 0.27 mmol) was stirred at 70° C. for 16 h at which time the solution was cooled to rt and filtered. The filtrate was concentrated and purified using preparatory HPLC. MS (ESI): mass calcd. for C₂₉H₄₀F₃N₅O₄S, 611.28; m/z found, 612.3 [M+H]⁺. ¹H NMR (mixture of rotamers): 8.05-7.98 (m, 0.3H), 7.96-7.92 (m, 0.7H), 7.87-7.77 (m, 1.7H), 7.74-7.68 (m, 0.3H), 4.77-4.64 (m, 1H), 4.55-4.46 (m, 1H), 4.42-4.28 (m, 2H), 4.28-4.14 (m, 2H), 4.12-3.69 (m, 6H), 3.69-3.39 (m, 8H), 3.40-3.13 (m, 5H), 3.12-2.83 (m, 4H), 2.02-1.66 (m, 5H), 1.62-1.47 (m, 1H).

The compounds in Examples 239-241 were prepared using methods similar to those in Example 238.

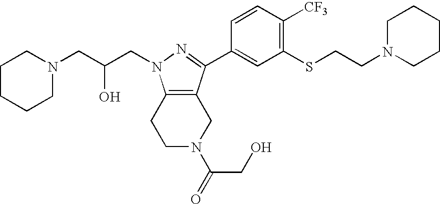

### Example 239

### 1-[5-(Hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol

MS (ESI): mass calcd. for C₃₀H₄₂F₃N₅O₃S, 609.30; m/z found, 610.3 [M+H]⁺. ¹H NMR: 7.93 (d, J=11.8, 1H), 7.74 (d, J=8.0, 1H), 7.61-7.53 (m, 1H), 4.83 (d, J=1.9, 1H), 4.70-4.68 (m, 1H), 4.37-4.29 (m, 2H), 4.23-4.14 (m, 2H), 4.08-3.99 (m, 1H), 3.99-3.88 (m, 1H), 3.84-3.68 (m, 1H), 3.28-3.18 (m, 2H), 3.02-2.89 (m, 2H), 2.88-2.80 (m, 1H), 2.68-2.60 (m, 2H), 2.56-2.34 (m, 9H), 1.65-1.54 (m, 8H), 1.51-1.41 (m, 4H), 1.36-1.25 (m, 1H), 0.94-0.83 (m, 1H).

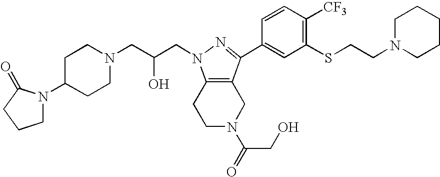

### Example 240

### 1-(1-{2-Hydroxy-3-[5-(hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one

MS (ESI): mass calcd. for C₃₄H₄₇F₃N₆O₄S, 692.33; m/z found, 693.3 [M+H]⁺. ¹H NMR: 7.97 (d, J=18.4, 1H), 7.75 (d, J=8.3, 1H), 7.65-7.55 (m, 1H), 4.81 (s, 1H), 4.47-4.40 (m, 1H), 4.36 (s, 1.3H), 4.32 (s, 0.7H), 4.22 (dd, J=14.2, 3.6, 1H), 4.13-3.99 (m, 2H), 3.74 (t, J=5.6, 1H), 3.57 (d, J=10.2, 1H), 3.54-3.46 (m, 3H), 3.43 (t, J=7.1, 2H), 3.24-3.02 (m, 7H), 3.02-2.73 (m, 5H), 2.68 (d, J=15.2, 2H), 2.59 (d, J=15.2, 2H), 2.36 (t, J=8.1, 2H), 2.15-1.96 (m, 4H), 1.84-1.71 (m, 6H), 1.59 (s, 2H).

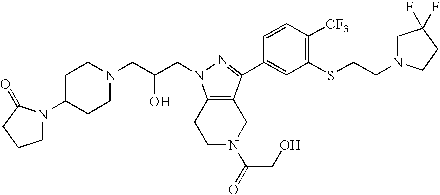

### Example 241

### 1-[1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)piperidin-4-yl]pyrrolidin-2-one

MS (ESI): mass calcd. for C₃₃H₄₃F₅N₆O₄S, 714.2; m/z found, 715.2 [M+H]⁺. ¹H NMR: 8.15-7.95 (m, 1H), 7.81 (d, J=4.5, 1H), 7.75-7.60 (m, 1H), 5.49 (s, 1H), 4.66-3.08 (m, 27H), 2.95 (d, J=52.1, 2H), 2.69 (s, 2H), 2.39 (t, J=7.4, 2H), 2.31-1.79 (m, 6H).

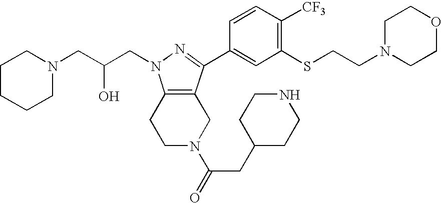

### Example 242

### 1-[3-{3-[(2-Morpholin-4-ylethyl)sulfanyl]-4-(trifluoromethyl)phenyl}-5-(piperidin-4-ylacetyl)-4,5,6,7-tetrahydro-1H-pyrazolo[4,3-c]pyridin-1-yl]-3-piperidin-1-ylpropan-2-ol

A. 4-(2-{1-(2-Hydroxy-3-piperidin-1-yl-propyl)-3-[3-(2-morpholin-4-yl-ethylsulfanyl)-4-trifluoromethyl-phenyl]-1,4,6,7-tetrahydro-pyrazolo[4,3-c]pyridin-5-yl}-2-oxo-ethyl)-piperidine-1-carboxylic acid tert-butyl ester. This compound was prepared using methods analogous to those described in Example 238, with the appropriate substituent changes. The crude product was used without purification. MS (ESI): mass calcd. for C₃₉H₅₇F₃N₆O₅S, 778.41; m/z found, 779.4 [M+H]⁺.

B. 1-[3-{3-[(2-Morpholin-4-ylethyl)sulfanyl]-4-(trifluoromethyl)phenyl}-5-(piperidin-4-ylacetyl)-4,5,6,7-tetrahydro-1H-pyrazolo[4,3-c]pyridin-1-yl]-3-piperidin-1-ylpropan-2-ol. To a solution of 4-(2-{1-(2-hydroxy-3-piperidin-1-yl-propyl)-3-[3-(2-morpholin-4-yl-ethylsulfanyl)-4-trifluoromethyl-phenyl]-1,4,6,7-tetrahydro-pyrazolo[4,3-c]pyridin-5-yl}-2-oxo-ethyl)-piperidine-1-carboxylic acid tert-butyl ester (30 mg, 0.039 mmol) in CH₂Cl₂ (2 mL) was added TFA (1 mL). The solution was stirred for 3 h and blown dry under a stream of N₂. Purification using reverse phase HPLC provided the title compound (16 mg, 61%). MS (ESI): mass calcd. for C₃₄H₄₉F₃N₆O₃S, 678.35; m/z found, 679.3 [M+H]⁺. ¹H NMR (mixture of rotamers): 8.10-8.06 (m, 0.25H), 7.97-7.93 (m, 0.75H), 7.86-7.81 (m, 1H), 7.80-7.75 (m, 0.75H), 7.67-7.63 (m, 0.25H), 4.91-4.72 (m, 2H), 4.56-4.47 (m, 1H), 4.28-4.12 (m, 2H), 4.12-3.65 (m, 6H), 3.66-3.25 (m, 12H), 3.24-3.13 (m, 2H), 3.12-2.81 (m, 6H), 2.60-2.46 (m, 2H), 2.23-2.10 (m, 1H), 2.08-1.69 (m, 7H), 1.62-1.37 (m, 3H).

Examples 243-247 were prepared using methods similar to those described in Example 234, substituting the appropriate acid chloride for the sulfonyl chloride.

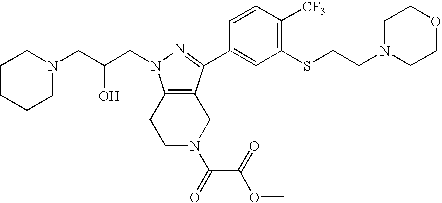

### Example 243

### Methyl[1-(2-hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl](oxo)acetate

MS (ESI): mass calcd. for C₃₀H₄₀F₃N₅O₅S, 639.27; m/z found, 640.3 [M+H]⁺. ¹H NMR (mixture of rotamers): 8.00-7.92 (m, 1H), 7.88-7.77 (m, 1.8H), 7.67-7.62 (m, 0.2H), 4.89-4.74 (m, 1H), 4.56-4.45 (m, 1H), 4.31-4.14 (m, 2H), 4.13-3.71 (m, 9H), 3.69-3.41 (m, 8H), 3.39-3.27 (m, 3H), 3.26-2.91 (m, 6H), 2.02-1.66 (m, 5H), 1.62-1.48 (m, 1H).

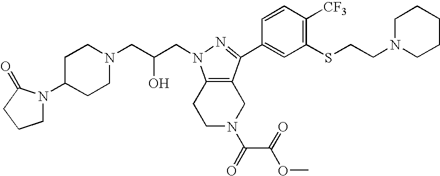

### Example 244

### Methyl(1-{2-hydroxy-3-[4-(2-oxopyrrolidin-1-yl)piperidin-1-yl]propyl}-3-{3-[(2-piperidin-1-ylethyl)sulfanyl]-4-(trifluoromethyl)phenyl}-1,4,6,7-tetrahydro-5H-pyrazolo[4,3-c]pyridin-5-yl)(oxo)acetate

MS (ESI): mass calcd. for C₃₅H₄₇F₃N₆O₅S, 720.33; m/z found, 721.3 [M+H]⁺. ¹H NMR: 7.96 (s, 0.2H), 7.93 (s, 0.8H), 7.85-7.77 (m, 2H), 4.49 (s, 1H), 4.29-4.21 (m, 1H), 4.19 (dd, J=14.4, 6.4, 1H), 4.16-4.07 (m, 1H), 3.94-3.84 (m, 3H), 3.84-3.78 (m, 2H), 3.71 (t, J=15.1, 2H), 3.59 (d, J=11.4, 2H), 3.51-3.43 (m, 3H), 3.43-3.39 (m, 2H), 3.37-3.37 (m, 2H), 3.36-3.32 (m, 3H), 3.28-3.18 (m, 2H), 3.13 (t, J=12.9, 1H), 3.05-2.90 (m, 4H), 2.39 (t, J=8.1, 2H), 2.19-2.08 (m, 1H), 2.04 (td, J=15.1, 7.5, 3H), 1.99-1.86 (m, 4H), 1.87-1.67 (m, 3H), 1.57-1.42 (m, 1H).

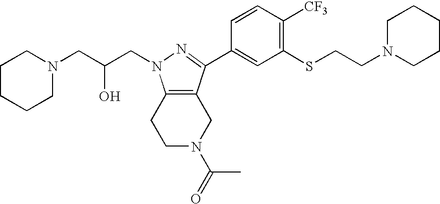

### Example 245

### 1-(5-Acetyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol

MS (ESI): mass calcd. for C₃₀H₄₂F₃N₅O₂S, 593.30; m/z found, 594.3 [M+H]⁺. ¹H NMR: 8.07-7.90 (m, 1H), 7.84-7.81 (m, 1H), 7.78-7.65 (m, 1H), 4.95-4.93 (m, 1H), 4.87-4.76 (m, 2H), 4.53-4.45 (m, 1H), 4.23 (dd, J=14.4, 4.1, 1H), 4.17 (dd, J=14.4, 6.7, 1H), 4.00-3.82 (m, 2H), 3.62-3.52 (m, 4H), 3.50-3.45 (m, 2H), 3.39-3.32 (m, 3H), 3.18 (dd, J=13.3, 10.8, 1H), 3.08-3.01 (m, 1H), 3.01-2.82 (m, 5H), 2.24-2.16 (m, 3H), 1.97-1.70 (m, 10H), 1.57-1.44 (m, 2H).

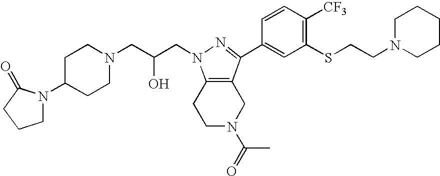

### Example 246

### 1-{1-[3-(5-Acetyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}pyrrolidin-2-one

MS (ESI): mass calcd. for C₃₄H₄₇F₃N₆O₃S, 676.34; m/z found, 677.3 [M+H]⁺. ¹H NMR: 8.20-8.06 (m, 1H), 7.87 (d, J=7.2, 1H), 7.72 (d, J=7.1, 1H), 4.96-4.83 (m, 2H), 4.77-4.65 (m, 1H), 4.52-4.39 (m, 1H), 4.40-4.29 (m, 1H), 4.25-4.12 (m, 1H), 4.09-3.93 (m, 2H), 3.87-3.73 (m, 2H), 3.72-3.55 (m, 4H), 3.46-3.17 (m, 9H), 3.16-2.92 (m, 4H), 2.50-2.40 (m, 2H), 2.30-2.26 (m, 3H), 2.21-2.11 (m, 1H), 2.12-2.01 (m, 2H), 2.00-1.78 (m, 7H), 1.59-1.42 (m, 1H).

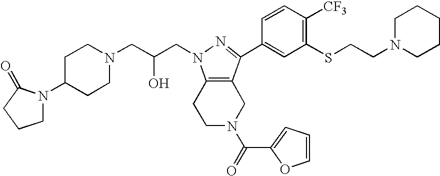

### Example 247

### 1-(1-{3-[5-(Furan-2-ylcarbonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-2-hydroxypropyl}piperidin-4-yl)pyrrolidin-2-one

MS (ESI): mass calcd. for C₃₇H₄₇F₃N₆O₄S, 728.33; m/z found, 729.3 [M+H]⁺. ¹H NMR: 8.14-8.06 (m, 1H), 7.86 (d, J=7.1, 1H), 7.79-7.66 (m, 2H), 7.15 (d, J=2.2, 1H), 6.66-6.60 (m, 1H), 4.73-4.65 (m, 1H), 4.48-4.26 (m, 2H), 4.25-4.10 (m, 3H), 3.85-3.73 (m, 2H), 3.69-3.57 (m, 5H), 3.51-3.44 (m, 3H), 3.42-3.33 (m, 3H), 3.27-3.19 (m, 2H), 3.19-3.07 (m, 2H), 3.06-2.97 (m, 2H), 2.43 (t, J=7.3, 2H), 2.29-2.00 (m, 5H), 1.99-1.78 (m, 8H), 1.57-1.46 (m, 1H).

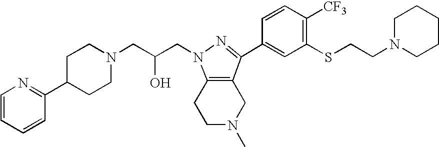

### Example 248

### 1-(5-Methyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol

To a solution of 1-{3-[3-(2-piperidin-1-yl-ethylsulfanyl)-4-trifluoromethyl-phenyl]-4,5,6,7-tetrahydro-pyrazolo[4,3-c]pyridin-1-yl}-3-(3′,4′,5′,6′-tetrahydro-2′H-[2,4′]bipyridinyl-1′-yl)-propan-2-ol (34 mg, 0.050 mmol) in acetonitrile (0.32 mL) was added formaldehyde (37% solution in H₂O; 0.054 mL, 0.27 mmol) and NaCNBH₃ (7 mg, 0.1 mmol). The reaction mixture was stirred for 4 h, filtered, diluted with DMF and purified using reverse phase HPLC to provide the title compound (29 mg, 91%). MS (ESI): mass calcd. for C₃₄H₄₅F₃N₆OS, 642.33; m/z found, 643.3 [M+H]⁺. ¹H NMR: 8.71-8.68 (m, 1H), 8.27 (t, J=6.4, 1H), 7.99 (s, 1H), 7.83 (d, J=8.2, 1H), 7.75 (d, J=8.0, 1H), 7.72-7.67 (m, 1H), 7.64 (d, J=8.2, 1H), 4.82-4.67 (m, 1H), 4.65-4.54 (m, 2H), 4.54-4.38 (m, 1H), 4.31 (dd, J=14.4, 3.4, 1H), 4.23 (dd, J=14.4, 7.0, 1H), 3.91-3.75 (m, 3H), 3.57 (d, J=11.7, 3H), 3.48 (dd, J=9.3, 6.5, 3H), 3.45-3.39 (m, 1H), 3.38-3.31 (m, 6H), 3.27-3.20 (m, 1H), 3.11 (s, 3H), 2.96 (t, J=11.7, 2H), 2.33-2.11 (m, 4H), 1.92 (d, J=14.2, 2H), 1.86-1.70 (m, 3H), 1.55-1.40 (m, 1H).

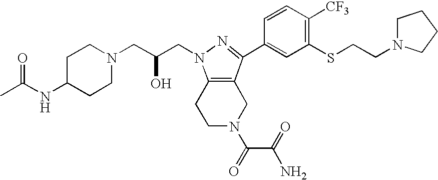

### Example 249

### 2-(1-{(2S)-3-[4-(Acetylamino)piperidin-1-yl]-2-hydroxypropyl}-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide

A mixture of N-[1-((2S)-2-hydroxy-3-{3-[3-(2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-acetamide 74.9 mg, 0.14 mmol) (prepared analogously to Example 233), oxamic acid (52 mg, 0.58 mmol), HATU (110 mg, 0.29 mmol), HOAt (0.5 M in DMF; 0.6 mL), and DIPEA (0.075 mL, 0.43 mmol) was stirred at 70° C. for 24.5 h at which time the solution was cooled to rt, and filtered. The filtrate was concentrated and purified using preparatory HPLC. MS (ESI): mass calcd. for C₃₁H₄₂F₃N₇O₄S, 665.30; m/z found, 666.3 [M+H]⁺. ¹H NMR: 8.05-7.64 (m, 3H), 5.01 (d, J=3.9, 1H), 4.87-4.79 (m, 2H), 4.53-4.45 (m, 1H), 4.23 (dd, J=14.5, 4.3, 1H), 4.17 (dd, J=13.6, 6.9, 1H), 4.04-3.84 (m, 3H), 3.80-3.70 (m, 2H), 3.70-3.61 (m, 2H), 3.55-3.40 (m, 5H), 3.37-3.32 (m, 1H), 3.27-3.05 (m, 5H), 3.05-2.98 (m, 1H), 2.97-2.90 (m, 1H), 2.22-2.05 (m, 4H), 2.06-1.95 (m, 4H), 1.93 (s, 3H), 1.82 (dd, J=24.8, 12.5, 1H), 1.74 (dd, J=25.3, 12.6, 1H).

The compounds in Examples 250-251 were prepared using methods similar to those described for Example 249.

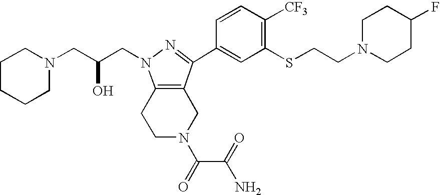

### Example 250

### 2-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-2-oxoacetamide

MS (ESI): mass calcd. for C₃₀H₄₀F₄N₆O₃S, 640.28; m/z found, 641.2 [M+H]⁺. ¹H NMR: 7.95-7.81 (m, 1H), 7.72 (dd, J=11.5, 8.3, 1H), 7.59 (dd, J=29.9, 8.0, 1H), 4.95-4.89 (m, 2H), 4.78 (d, J=6.0, 2H), 4.56-4.42 (m, 1H), 4.19 (dd, J=14.4, 3.8, 1H), 4.12 (dd, J=14.0, 6.9, 1H), 3.93-3.83 (m, 2H), 3.68-3.57 (m, 1H), 3.58-3.44 (m, 6H), 3.37-3.26 (m, 3H), 3.16-3.05 (m, 2H), 3.05-2.83 (m, 4H), 2.27-2.17 (m, 1H), 2.16-2.01 (m, 3H), 2.00-1.88 (m, 1H), 1.88-1.64 (m, 5H), 1.52-1.38 (m, 1H), 1.26-1.14 (m, 1H).

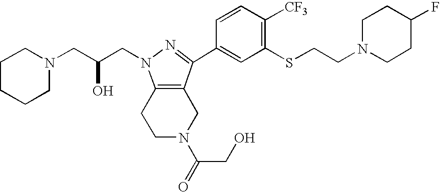

### Example 251

### (2S)-1-{3-[3-{[2-(4-Fluoropiperidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-piperidin-1-ylpropan-2-ol

MS (ESI): mass calcd. for C₃₀H₄₁F₄N₅O₃S, 627.29; m/z found, 628.3 [M+H]⁺. ¹H NMR: 8.04-7.88 (m, 1H), 7.82 (d, J=8.3, 1H), 7.79-7.65 (m, 1H), 4.88-4.77 (m, 2H), 4.53-4.43 (m, 1H), 4.39-4.28 (m, 2H), 4.23 (dd, J=14.4, 4.2, 1H), 4.16 (dd, J=14.4, 6.7, 1H), 3.83-3.71 (m, 2H), 3.62-3.47 (m, 6H), 3.47-3.37 (m, 3H), 3.34-3.31 (m, 1H), 3.17 (dd, J=13.3, 10.8, 2H), 3.11-3.00 (m, 2H), 3.00-2.92 (m, 3H), 2.91-2.83 (m, 1H), 2.32-1.98 (m, 4H), 1.96-1.72 (m, 6H), 1.57-1.47 (m, 1H).

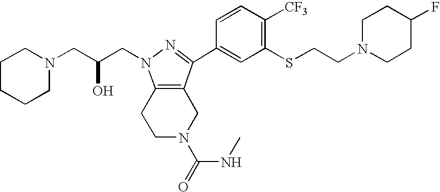

### Example 252

### 3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-methyl-1,4,6,7-tetrahydro-5H-pyrazolo[4,3-c]pyridine-5-carboxamide

To a solution of (2S)-1-(3-{3-[2-(4-fluoro-piperidin-1-yl)-ethylsulfanyl]-4-trifluoromethyl-phenyl}-4,5,6,7-tetrahydro-pyrazolo[4,3-c]pyridin-1-yl)-3-piperidin-1-yl-propan-2-ol (64 mg, 0.112 mmol) (prepared analogously to Example 233) in CH₂Cl₂ (0.94 mL) was added methylisocyanate (7.6, 0.134 mmol). After 3 h, the mixture was concentrated, dissolved in DMF, and filtered. The filtrate was concentrated and purified using preparatory HPLC to afford the citric acid salt (6.5 mg, 0.008 mmol). MS (ESI): mass calcd. for C₃₀H₄₂F₄N₆O₂S, 626.30; m/z found, 627.3 [M+H]⁺. ¹H NMR: 7.99 (s, 1H), 7.75 (d, J=8.2, 1H), 7.60 (d, J=8.2, 1H), 4.79 (d, J=48.6, 1H), 4.63 (s, 2H), 4.55-4.48 (m, 1H), 4.21 (dd, J=14.4, 4.0, 2H), 4.12 (dd, J=14.3, 7.0, 2H), 3.75 (dd, J=10.4, 5.4, 2H), 3.48-3.38 (m, 2H), 3.14 (dd, J=13.2, 10.5, 2H), 3.10-2.97 (m, 5H), 2.92-2.76 (m, 2H), 2.75-2.68 (m, 5H), 2.67-2.58 (m, 3H), 2.18-2.02 (m, 2H), 2.02-1.91 (m, 2H), 1.92-1.76 (m, 4H), 1.68-1.57 (m, 2H).

Examples 253-254 were prepared using methods similar to those described in Example 110, with the appropriate substituent changes.

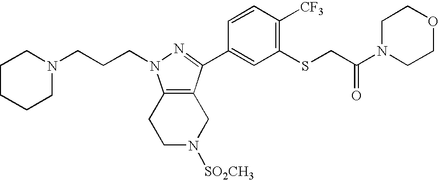

### Example 253

### 5-(Methylsulfonyl)-3-{3-[(2-morpholin-4-yl-2-oxoethyl)sulfanyl]-4-(trifluoromethyl)phenyl}-1-(3-piperidin-1-ylpropyl)-4,5,6,7-tetrahydro-1H-pyrazolo[4,3-c]pyridine

MS (ESI): mass calcd. for C₂₈H₃₈F₃N₅O₄S₂, 629.23; m/z found, 630.2 [M+H]⁺. ¹H NMR (CDCl₃): 7.93 (s, 1H), 7.69 (t, J=8.4, 1H), 7.53 (t, J=7.2, 1H), 4.59 (s, 2H), 4.16 (t, J=6.4, 2H), 3.82 (s, 2H), 3.72-3.69 (m, 1H), 3.66 (t, J=5.7, 2H), 3.63-3.57 (m, 2H), 3.18-3.12 (m, 2H), 3.10 (s, 1H), 2.97 (s, 2H), 2.96 (d, J=3.5, 2H), 2.86 (t, J=5.4, 2H), 2.79-2.59 (m, 7H), 2.46-2.34 (m, 2H), 2.04-1.91 (m, 2H), 1.91-1.82 (m, 3H), 1.48-1.32 (m, 1H).

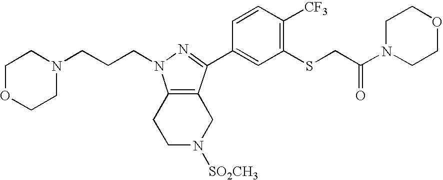

### Example 254

### 5-(Methylsulfonyl)-3-{3-[(2-morpholin-4-yl-2-oxoethyl)sulfanyl]-4-(trifluoromethyl)phenyl}-1-(3-morpholin-4-ylpropyl)-4,5,6,7-tetrahydro-1H-pyrazolo[4,3-c]pyridine

MS (ESI): mass calcd. for C₂₇H₃₆F₃N₅O₅S₂, 631.21; m/z found, 632.2 [M+H]⁺. ¹H NMR: 7.95 (d, J=11.3, 1H), 7.75 (t, J=7.7, 1H), 7.66 (d, J=8.0, 1H), 4.58 (d, J=3.0, 2H), 4.18 (t, J=6.6, 2H), 4.01 (d, J=6.0, 2H), 3.72-3.62 (m, 8H), 3.60-3.55 (m, 2H), 3.17-3.09 (m, 2H), 2.98 (s, 3H), 2.96-2.95 (m, 2H), 2.95 (s, 2H), 2.47-2.38 (m, 4H), 2.37-2.32 (m, 2H), 2.13-2.05 (m, 2H).

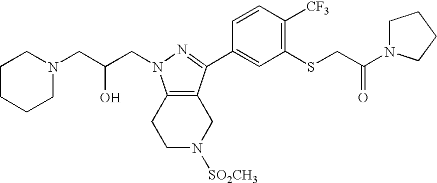

### Example 255

### 1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol

A. [5-(5-Methanesulfonyl-4,5,6,7-tetrahydro-1H-pyrazolo[4,3-c]pyridin-3-yl)-2-trifluoromethyl-phenylsulfanyl]-acetic acid ethyl ester. This compound was prepared using methods similar to those described in Example 1, Steps A-C, with the appropriate substituent changes. MS (ESI): mass calcd. for C₁₈H₂₀F₃N₃O₄S₂, 463.08; m/z found, 464.1 [M+H]⁺. ¹H NMR (CDCl₃): 7.87 (s, 1H), 7.70 (d, J=8.2, 1H), 7.52 (d, J=8.1, 1H), 4.59 (s, 2H), 4.20-4.14 (m, 2H), 3.72-3.65 (m, 4H), 2.98-2.91 (m, 6H), 1.23-1.19 (m, 3H).

B. 2-[5-(5-Methanesulfonyl-4,5,6,7-tetrahydro-1H-pyrazolo[4,3-c]pyridin-3-yl)-2-trifluoromethyl-phenylsulfanyl]-1-pyrrolidin-1-yl-ethanone. A solution of [5-(5-methanesulfonyl-4,5,6,7-tetrahydro-1H-pyrazolo[4,3-c]pyridin-3-yl)-2-trifluoromethyl-phenylsulfanyl]-acetic acid ethyl ester (110 mg, 0.24 mmol) in pyrrolidine (1 mL, 12 mmol) was heated at 220° C. in a microwave reactor for 40 min. The reaction mixture was poured into a solution of cold satd. aq. NH₄Cl. The resultant precipitate was collected by filtration (90 mg, 69%) and used without further purification. ¹H NMR (DMSO-d₆): 7.84-7.72 (m, 2H), 7.63-7.60 (m, 1H), 4.53 (s, 2H), 4.13 (s, 2H), 3.59-3.47 (m, 4H), 3.41-3.29 (m, 2H), 3.01 (s, 3H), 2.85 (t, J=5.5, 2H), 1.96-1.85 (m, 2H), 1.82-1.76 (m, 2H).

C. 1-[5-(Methylsulfonyl)-3-[3-[(2-oxo-2-pyrrolidin-1-ylethyl)sulfanyl]-4-(trifluoromethyl)phenyl]-4,5,6,7-tetrahydro-1H-pyrazolo[4,3-c]pyridin-1-yl]-3-piperidin-1-ylpropan-2-ol. This compound was prepared using methods similar to those described in Example 61, Steps B and C, with the appropriate substituent changes. MS (ESI): mass calcd. for C₂₈H₃₈F₃N₅O₄S₂, 629.23; m/z found, 630.2 [M+H]⁺. ¹H NMR: 8.06 (s, 1H), 7.76 (d, J=8.2, 1H), 7.67 (d, J=8.1, 1H), 4.61-4.54 (m, 2H), 4.53-4.43 (m, 1H), 4.27-4.11 (m, 2H), 3.92-3.86 (m, 2H), 3.70-3.59 (m, 2H), 3.58-3.50 (m, 4H), 3.41 (t, J=6.9, 2H), 3.24-3.13 (m, 1H), 3.12-3.01 (m, 1H), 3.01-2.97 (m, 1H), 2.99 (s, 3H), 2.95-2.88 (m, 2H), 2.00-1.90 (m, 4H), 1.90-1.81 (m, 4H), 1.81-1.67 (m, 2H), 1.59-1.42 (m, 1H).

Examples 256-262 were prepared using methods similar to those described in Example 255, with the appropriate substituent changes.

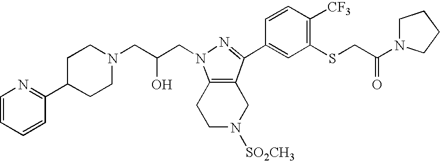

### Example 256

### 1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol

MS (ESI): mass calcd. for C₃₃H₄₁F₃N₆O₄S₂, 706.26; m/z found, 707.2 [M+H]⁺. ¹H NMR: 8.65-8.63 (m, 1H), 8.16-8.11 (m, 1H), 8.07 (s, 1H), 7.76 (d, J=8.2, 1H), 7.68 (d, J=8.2, 1H), 7.62 (d, J=7.9, 1H), 7.60-7.57 (m, 1H), 4.59 (s, 1H), 4.57-4.46 (m, 1H), 4.37-4.12 (m, 2H), 4.01-3.86 (m, 2H), 3.85-3.72 (m, 2H), 3.72-3.57 (m, 3H), 3.54 (t, J=6.8, 2H), 3.41 (t, J=7.0, 2H), 3.38-3.32 (m, 2H), 3.29-3.13 (m, 3H), 3.01-2.99 (m, 1H), 2.99 (s, 3H), 2.94-2.89 (m, 1H), 2.29-2.06 (m, 4H), 2.03-1.91 (m, 2H), 1.91-1.82 (m, 2H).

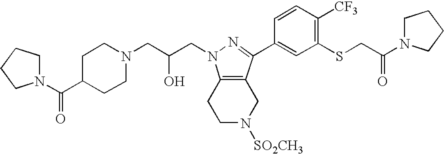

### Example 257

### 1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol

MS (ESI): mass calcd. for C₃₃H₄₅F₃N₆O₅S₂, 726.28; m/z found, 727.2 [M+H]⁺. ¹H NMR: 8.05 (s, 1H), 7.75 (d, J=8.2, 1H), 7.67 (d, J=8.0, 1H), 4.58 (s, 2H), 4.54-4.46 (m, 1H), 4.30-4.13 (m, 2H), 3.89 (s, 2H), 3.75-3.61 (m, 4H), 3.59-3.51 (m, 4H), 3.46-3.37 (m, 4H), 3.29-3.21 (m, 1H), 3.20-3.12 (m, 1H), 3.11-3.03 (m, 1H), 2.99 (s, 3H), 2.98-2.96 (m, 2H), 2.90-2.79 (m, 1H), 2.12-1.91 (m, 8H), 1.92-1.76 (m, 4H).

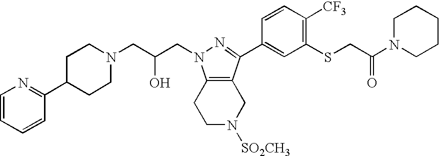

### Example 258

### 1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol

MS (ESI): mass calcd. for C₃₄H₄₃F₃N₆O₄S₂, 720.27; m/z found, 721.2 [M+H]⁺. ¹H NMR: 8.65-8.60 (m, 1H), 8.10 (s, 1H), 8.06 (t, J=7.7, 1H), 7.78 (d, J=8.2, 1H), 7.68 (d, J=8.1, 1H), 7.60-7.48 (m, 2H), 4.62 (s, 2H), 4.59-4.47 (m, 1H), 4.41-4.03 (m, 3H), 4.00 (s, 2H), 3.86-3.75 (m, 2H), 3.73-3.64 (m, 2H), 3.63-3.47 (m, 5H), 3.46-3.38 (m, 1H), 3.25-3.12 (m, 2H), 3.03-3.01 (m, 1H), 3.01 (s, 3H), 2.96-2.89 (m, 1H), 2.37-2.03 (m, 4H), 1.79-1.58 (m, 4H), 1.58-1.48 (m, 2H).

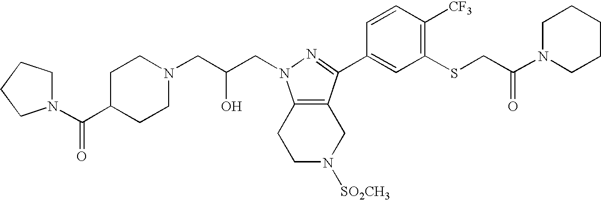

### Example 259

### 1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol

MS (ESI): mass calcd. for C₃₄H₄₇F₃N₆O₅S₂, 740.30; m/z found, 741.3 [M+H]⁺. ¹H NMR: 8.07 (s, 1H), 7.76 (d, J=8.2, 1H), 7.65 (d, J=7.4, 1H), 4.59 (s, 2H), 4.55-4.42 (m, 1H), 4.37-4.07 (m, 2H), 4.05-3.89 (m, 2H), 3.77-3.61 (m, 4H), 3.60-3.45 (m, 8H), 3.45-3.36 (m, 2H), 3.26-3.03 (m, 2H), 2.99 (s, 3H), 2.98-2.75 (m, 3H), 2.10-1.94 (m, 5H), 1.93-1.76 (m, 3H), 1.75-1.57 (m, 4H), 1.51 (s, 2H).

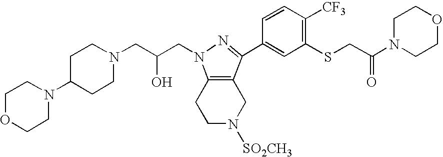

### Example 260

### 1-[5-(Methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol

MS (ESI): mass calcd. for C₃₂H₄₅F₃N₆O₆S₂, 730.28; m/z found, 731.2 [M+H]⁺. ¹H NMR: 8.04 (s, 1H), 7.77 (d, J=8.3, 1H), 7.67 (d, J=8.3, 1H), 4.59 (s, 2H), 4.56-4.46 (m, 1H), 4.30-4.14 (m, 2H), 4.00 (s, 2H), 3.99-3.86 (m, 4H), 3.83 (d, J=11.7, 2H), 3.71-3.64 (m, 3H), 3.64-3.60 (m, 3H), 3.60-3.53 (m, 4H), 3.53-3.44 (m, 1H), 3.44-3.27 (m, 5H), 3.27-3.08 (m, 3H), 2.99 (s, 3H), 2.98-2.95 (m, 2H), 2.43 (t, J=14.4, 2H), 2.23-1.98 (m, 2H).

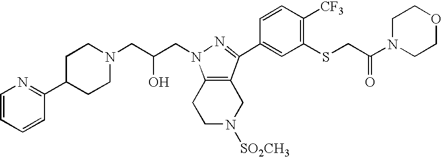

### Example 261

### 1-[5-(Methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol

MS (ESI): mass calcd. for C₃₃H₄₁F₃N₆O₅S₂, 722.25; m/z found, 723.2 [M+H]⁺. ¹H NMR: 8.70-8.66 (m, 1H), 8.20 (t, J=7.2, 1H), 8.06 (s, 1H), 7.78 (d, J=8.2, 1H), 7.74-7.62 (m, 3H), 4.62 (s, 2H), 4.60-4.53 (m, 1H), 4.34-4.19 (m, 2H), 4.02 (s, 2H), 3.89-3.76 (m, 2H), 3.74-3.52 (m, 10H), 3.50-3.38 (m, 2H), 3.32-3.15 (m, 3H), 3.03-3.01 (m, 2H), 3.01 (s, 3H), 2.42-1.98 (m, 4H).

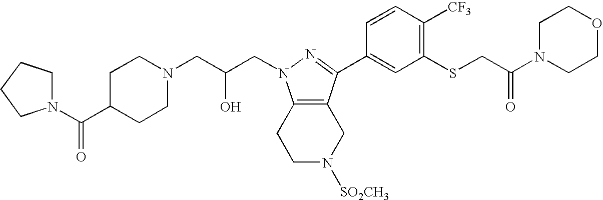

### Example 262

### 1-[5-(Methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol

MS (ESI): mass calcd. for C₃₃H₄₅F₃N₆O₆S₂, 742.28; m/z found, 743.4 [M+H]⁺. ¹H NMR: 8.09-8.03 (m, 1H), 7.76 (d, J=8.2, 1H), 7.65 (d, J=8.2, 1H), 4.58 (s, 2H), 4.54-4.45 (m, 1H), 4.31-4.14 (m, 2H), 4.00 (s, 2H), 3.78-3.65 (m, 5H), 3.65-3.61 (m, 4H), 3.60-3.57 (m, 2H), 3.57-3.53 (m, 2H), 3.54-3.43 (m, 2H), 3.43-3.28 (m, 4H), 3.28-3.15 (m, 2H), 3.16-3.07 (m, 1H), 3.06-2.94 (m, 8H), 2.33-2.15 (m, 2H), 2.16-2.00 (m, 1H), 1.95-1.70 (m, 1H).

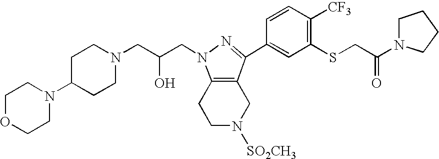

### Example 263

### 1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol

A. (5-{1-[2-Hydroxy-3-(4-morpholin-4-yl-piperidin-1-yl)-propyl]-5-methanesulfonyl-4,5,6,7-tetrahydro-1H-pyrazolo[4,3-c]pyridin-3-yl}-2-trifluoromethyl-phenylsulfanyl)-acetic acid ethyl ester. This compound was prepared using methods similar to those described in Example 63, using the appropriate substituent changes. MS (ESI): mass calcd. for C₃₀H₄₂F₃N₅O₆S₂, 689.25; m/z found, 690.3 [M+H]⁺. ¹H NMR: 7.97 (s, 1H), 7.78 (d, J=8.3, 1H), 7.69 (d, J=8.2, 1H), 4.58 (s, 2H), 4.53-4.45 (m, 1H), 4.30-4.20 (m, 2H), 4.19-4.14 (m, 2H), 3.95-3.82 (m, 4H), 3.79-3.61 (m, 4H), 3.27-3.03 (m, 8H), 3.01 (s, 3H), 2.43-2.22 (m, 2H), 2.12-1.86 (m, 4H), 1.18 (t, J=7.1, 3H).

B. 1-[5-(Methylsulfonyl)-3-{3-[(2-oxo-2-pyrrolidin-1-ylethyl)sulfanyl]-4-(trifluoromethyl)phenyl]-4,5,6,7-tetrahydro-1H-pyrazolo[4,3-c]pyridin-1-yl]-3-(4-morpholin-4-ylpiperidin-1-yl)propan-2-ol. This compound was prepared using methods similar to those described in Example 255, Step B, using the appropriate substituent changes. MS (ESI): mass calcd. for C₃₂H₄₅F₃N₆O₅S₂, 714.28; m/z found, 715.2 [M+H]⁺. ¹H NMR: 8.07 (s, 1H), 7.76 (d, J=8.2, 1H), 7.66 (d, J=7.8, 1H), 4.59 (s, 2H), 4.53-4.47 (m, 1H), 4.30-4.17 (m, 2H), 3.98 (s, 2H), 3.97-3.87 (m, 4H), 3.87-3.76 (m, 3H), 3.72-3.58 (m, 2H), 3.56-3.50 (m, 4H), 3.50-3.44 (m, 2H), 3.26-3.07 (m, 3H), 3.01-2.99 (m, 1H), 2.99 (s, 3H), 2.98-2.96 (m, 2H), 2.49-2.36 (m, 2H), 2.19-2.01 (m, 2H), 1.71-1.57 (m, 4H), 1.56-1.48 (m, 2H).

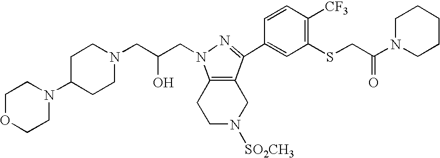

### Example 264

### 1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol

This compound was prepared using methods similar to those described in Example 263, with the appropriate substituent changes. MS (ESI): mass calcd. for C₃₃H₄₇F₃N₆O₅S₂, 728.30; m/z found, 729.3 [M+H]⁺. ¹H NMR: 8.07 (s, 1H), 7.76 (d, J=8.2, 1H), 7.67 (d, J=8.0, 1H), 4.58 (s, 2H), 4.54-4.47 (m, 1H), 4.29-4.17 (m, 2H), 3.99-3.91 (m, 3H), 3.89 (d, J=9.0, 2H), 3.87-3.76 (m, 3H), 3.71-3.59 (m, 2H), 3.53 (t, J=6.8, 2H), 3.51-3.43 (m, 2H), 3.41 (t, J=7.0, 2H), 3.39-3.32 (m, 3H), 3.30-3.28 (m, 2H), 3.28-3.09 (m, 3H), 2.99 (s, 3H), 2.98-2.96 (m, 2H), 2.51-2.31 (m, 2H), 2.21-1.99 (m, 3H), 1.97-1.92 (m, 2H), 1.89-1.82 (m, 2H).

Examples 265-271 were prepared using methods similar to those described in Example 228, with the appropriate substituent changes.

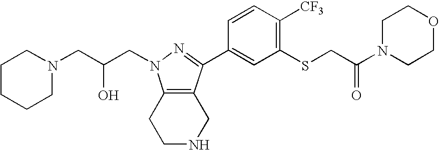

### Example 265

### 1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol

MS (ESI): mass calcd. for C₂₇H₃₆F₃N₅O₃S, 567.25; m/z found, 568.3 [M+H]⁺. ¹H NMR: 8.09 (s, 1H), 7.79 (d, J=8.2, 1H), 7.67 (d, J=8.1, 1H), 4.58-4.51 (m, 3H), 4.33-4.18 (m, 2H), 4.01 (s, 2H), 3.74-3.53 (m, 12H), 3.42-3.32 (m, 2H), 3.28-3.16 (m, 3H), 3.12-2.95 (m, 2H), 2.01-1.72 (m, 5H), 1.62-1.48 (m, 1H).

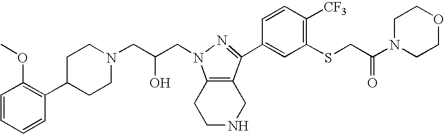

### Example 266

### 1-{4-[2-(Methyloxy)phenyl]piperidin-1-yl}-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propan-2-ol

MS (ESI): mass calcd. for C₃₄H₄₂F₃N₅O₄S, 673.29; m/z found, 674.3 [M+H]⁺. ¹H NMR: 8.10 (s, 1H), 7.81-7.63 (m, 2H), 7.26-7.18 (m, 1H), 7.16-7.12 (m, 1H), 6.99-6.89 (m, 2H), 4.58 (s, 3H), 4.34-4.19 (m, 2H), 4.00 (s, 2H), 3.83 (s, 3H), 3.80-3.45 (m, 12H), 3.45-3.11 (m, 7H), 2.14-1.89 (m, 4H).

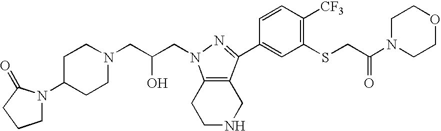

### Example 267

### 1-{1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one

MS (ESI): mass calcd. for C₃₁H₄₁F₃N₆O₄S, 650.29; m/z found, 651.3 [M+H]⁺. ¹H NMR: 8.09 (s, 1H), 7.78 (d, J=7.9, 1H), 7.65 (d, J=7.9, 1H), 4.64-4.46 (m, 3H), 4.28 (dd, J=25.9, 13.2, 2H), 4.17-4.10 (m, 1H), 4.02 (s, 2H), 3.81-3.72 (m, 2H), 3.71-3.68 (m, 2H), 3.65-3.58 (m, 6H), 3.57-3.52 (m, 3H), 3.44 (t, J=6.6, 2H), 3.41-3.36 (m, 1H), 3.29-3.12 (m, 4H), 2.39 (t, J=7.9, 2H), 2.24-2.08 (m, 2H), 2.08-2.00 (m, 4H), 1.98-1.88 (m, 2H).

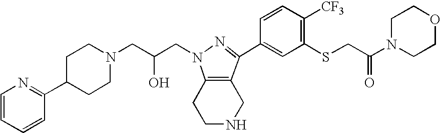

### Example 268

### 1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol

MS (ESI): mass calcd. for C₃₂H₃₉F₃N₆O₃S, 644.28; m/z found, 645.2 [M+H]⁺. ¹H NMR: 8.62-8.57 (m, 1H), 8.09 (s, 1H), 8.07-7.97 (m, 1H), 7.78 (d, J=8.2, 1H), 7.66 (d, J=8.3, 1H), 7.57-7.44 (m, 2H), 4.63-4.59 (m, 1H), 4.59 (s, 2H), 4.35-4.28 (m, 1H), 4.27-4.21 (m, 1H), 4.00 (s, 2H), 3.86-3.75 (m, 2H), 3.71-3.67 (m, 2H), 3.66-3.57 (m, 6H), 3.57-3.51 (m, 3H), 3.48-3.39 (m, 1H), 3.38-3.34 (m, 1H), 3.24-3.13 (m, 4H), 2.45-2.06 (m, 4H).

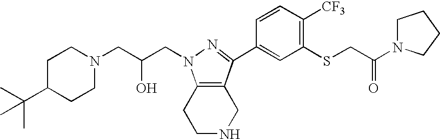

### Example 269

### 1-[4-(1,1-Dimethylethyl)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol

MS (ESI): mass calcd. for C₃₁H₄₄F₃N₅O₂S, 607.32; m/z found, 608.3 [M+H]⁺. ¹H NMR: 8.09 (s, 1H), 7.80-7.74 (m, 1H), 7.68-7.64 (m, 1H), 4.63-4.48 (m, 3H), 4.30-4.17 (m, 2H), 3.89 (s, 2H), 3.74-3.55 (m, 6H), 3.43-3.37 (m, 2H), 3.35-3.29 (m, 1H), 3.27-3.17 (m, 3H), 3.09-2.91 (m, 2H), 2.02-1.81 (m, 6H), 1.70-1.49 (m, 2H), 1.41-1.33 (m, 1H), 0.90 (s, 9H).

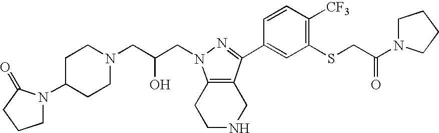

### Example 270

### 1-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one

MS (ESI): mass calcd. for C₃₁H₄₁F₃N₆O₃S, 634.29; m/z found, 635.3 [M+H]⁺. ¹H NMR: 8.09 (s, 1H), 7.77 (d, J=8.2, 1H), 7.66 (d, J=8.2, 1H), 4.61-4.55 (m, 1H), 4.57 (s, 2H), 4.34-4.22 (m, 2H), 4.18-4.09 (m, 1H), 3.92 (s, 2H), 3.81-3.71 (m, 2H), 3.62-3.56 (m, 4H), 3.49-3.36 (m, 5H), 3.28-3.15 (m, 4H), 2.39 (t, J=8.1, 2H), 2.24-2.08 (m, 2H), 2.08-2.00 (m, 2H), 1.99-1.94 (m, 3H), 1.94-1.91 (m, 1H), 1.91-1.84 (m, 3H).

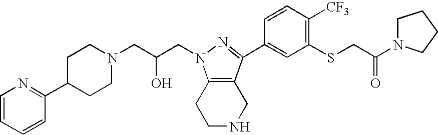

### Example 271

### 1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol

MS (ESI): mass calcd. for C₃₂H₃₉F₃N₆O₂S, 628.28; m/z found, 629.3 [M+H]⁺. ¹H NMR: 8.83 (d, J=5.4, 1H), 8.61-8.53 (m, 1H), 8.07 (s, 1H), 8.01 (d, J=8.1, 1H), 7.99-7.94 (m, 1H), 7.76 (d, J=8.3, 1H), 7.67 (d, J=8.2, 1H), 4.64-4.58 (m, 1H), 4.57 (s, 2H), 4.38-4.18 (m, 2H), 3.90 (s, 3H), 3.88-3.84 (m, 1H), 3.65-3.59 (m, 2H), 3.57 (t, J=6.8, 2H), 3.54-3.43 (m, 2H), 3.41 (t, J=6.9, 2H), 3.39-3.33 (m, 2H), 3.30-3.25 (m, 1H), 3.22 (t, J=6.1, 2H), 2.47-2.15 (m, 4H), 2.01-1.92 (m, 2H), 1.90-1.83 (m, 2H).

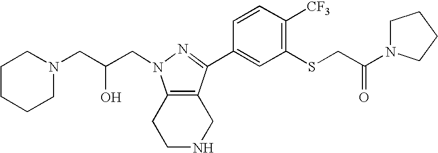

### Example 272

### 1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol

This compound was prepared using methods similar to those described in Example 228, substituting 20% TFA in CH₂Cl₂ for 4 M HCl in 1,4-dioxane. MS (ESI): mass calcd. for C₂₇H₃₆F₃N₅O₂S, 551.25; m/z found, 552.3 [M+H]⁺. ¹H NMR: 8.13-8.02 (m, 1H), 7.84-7.72 (m, 1H), 7.71-7.62 (m, 1H), 4.67-4.46 (m, 3H), 4.36-4.15 (m, 2H), 3.97-3.79 (m, 2H), 3.72-3.50 (m, 6H), 3.48-3.36 (m, 3H), 3.27-3.14 (m, 3H), 3.13-2.94 (m, 2H), 2.13-1.66 (m, 9H), 1.65-1.46 (m, 1H).

Examples 273-276 were prepared using methods similar to those described in Example 238, with the appropriate substituent changes.

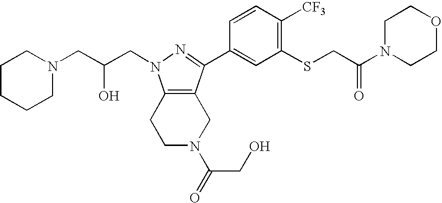

### Example 273

### 1-[5-(Hydroxyacetyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol

MS (ESI): mass calcd. for C₂₉H₃₈F₃N₅O₅S, 625.25; m/z found, 626.2 [M+H]⁺. ¹H NMR: 8.11-8.05 (m, 1H), 7.82-7.75 (m, 1H), 7.73-7.68 (m, 1H), 4.88-4.73 (m, 1H), 4.55-4.45 (m, 1H), 4.40-4.33 (m, 2H), 4.27-4.14 (m, 2H), 4.08-3.90 (m, 3H), 3.83-3.49 (m, 12H), 3.27-3.16 (m, 1H), 3.14-2.85 (m, 4H), 2.04-1.69 (m, 5H), 1.62-1.46 (m, 1H).

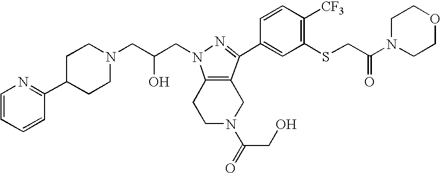

### Example 274

### 1-[5-(Hydroxyacetyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol

MS (ESI): mass calcd. for C₃₄H₄₁F₃N₆O₅S, 702.28; m/z found, 703.3 [M+H]⁺. ¹H NMR: 8.65-8.57 (m, 1H), 8.11-8.01 (m, 2H), 7.81-7.67 (m, 2H), 7.57-7.49 (m, 2H), 4.86-4.71 (m, 1H), 4.58-4.50 (m, 1H), 4.39-4.32 (m, 2H), 4.29-4.18 (m, 2H), 4.03 (s, 3H), 3.87-3.49 (m, 12H), 3.44-3.25 (m, 3H), 3.24-3.11 (m, 2H), 2.99-2.88 (m, 2H), 2.28-2.02 (m, 4H).

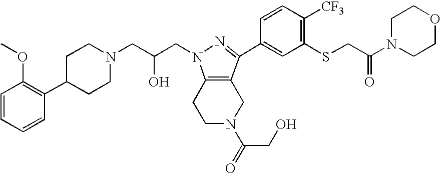

### Example 275

### 1-[5-(Hydroxyacetyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-{4-[2-(methyloxy)phenyl]piperidin-1-yl}propan-2-ol

MS (ESI): mass calcd. for C₃₆H₄₄F₃N₅O₆S, 731.30; m/z found, 732.3 [M+H]⁺. ¹H NMR: 8.10 (d, J=13.8, 1H), 7.80-7.75 (m, 1H), 7.72-7.68 (m, 1H), 7.25-7.18 (m, 1H), 7.14-7.11 (m, 1H), 6.98-6.95 (m, 1H), 6.94-6.89 (m, 1H), 4.90-4.83 (m, 1H), 4.79-4.74 (m, 1H), 4.56-4.49 (m, 1H), 4.38-4.32 (m, 2H), 4.28-4.17 (m, 2H), 4.03 (s, 3H), 3.83 (s, 3H), 3.80-3.46 (m, 11H), 3.44-3.11 (m, 5H), 3.00-2.83 (m, 2H), 2.10-1.89 (m, 4H).

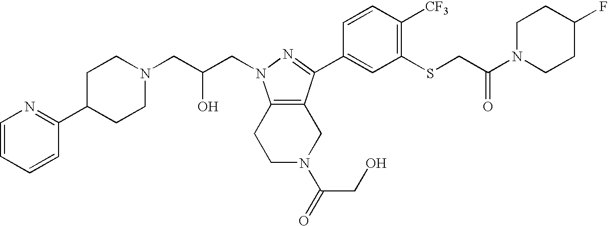

### Example 276

### 1-{3-[3-{[2-(4-Fluoropiperidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-(4-pyridin-2-ylpiperidin-1-yl)propan-2-ol

MS (ESI): mass calcd. for C₃₅H₄₄F₄N₆O₃S, 704.31; m/z found, 705.3 [M+H]⁺. ¹H NMR: 8.66-8.60 (m, 1H), 8.15-8.10 (m, 1H), 8.03-7.90 (m, 1H), 7.82 (d, J=8.3, 1H), 7.78 (d, J=7.9, 1H), 7.62 (d, J=7.9, 1H), 7.60-7.55 (m, 1H), 5.04-4.94 (m, 1H), 4.90-4.78 (m, 2H), 4.71 (s, 1H), 4.60-4.49 (m, 1H), 4.38 (s, 2H), 4.33-4.15 (m, 3H), 4.06-3.88 (m, 1H), 3.86-3.70 (m, 4H), 3.64-3.34 (m, 8H), 3.28-3.10 (m, 4H), 3.03-2.83 (m, 2H), 2.45-1.88 (m, 8H).

Examples 277-280 were prepared using methods similar to those described in Example 243, with the appropriate substituent changes.

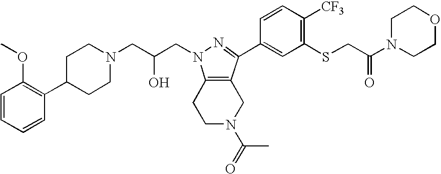

### Example 277

### 1-(5-Acetyl-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)-3-{4-[2-(methyloxy)phenyl]piperidin-1-yl}propan-2-ol

MS (ESI): mass calcd. for C₃₆H₄₄F₃N₅O₅S, 715.30; m/z found, 716.3 [M+H]⁺. ¹H NMR: 8.15-8.06 (m, 1H), 7.80-7.75 (m, 1H), 7.71-7.67 (m, 1H), 7.25-7.18 (m, 1H), 7.14-7.11 (m, 1H), 6.98-6.94 (m, 1H), 6.93-6.90 (m, 1H), 4.85-4.80 (m, 1H), 4.58-4.48 (m, 1H), 4.29-4.17 (m, 2H), 4.02 (s, 2H), 4.01-3.93 (m, 1H), 3.93-3.84 (m, 2H), 3.83 (s, 3H), 3.78-3.73 (m, 1H), 3.71-3.65 (m, 3H), 3.64-3.58 (m, 4H), 3.57-3.51 (m, 2H), 3.43-3.32 (m, 2H), 3.28-3.12 (m, 3H), 3.00-2.95 (m, 1H), 2.90-2.86 (m, 1H), 2.25-2.17 (m, 3H), 2.10-2.00 (m, 3H), 2.00-1.91 (m, 1H).

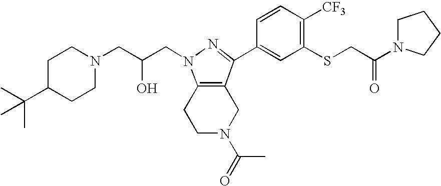

### Example 278

### 1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(1,1-dimethylethyl)piperidin-1-yl]propan-2-ol

MS (ESI): mass calcd. for C₃₃H₄₆F₃N₅O₃S, 649.33; m/z found, 650.46 [M+H]⁺. ¹H NMR: 8.14-8.03 (m, 1H), 7.79-7.74 (m, 1H), 7.72-7.65 (m, 1H), 4.86-4.78 (m, 2H), 4.53-4.43 (m, 1H), 4.27-4.12 (m, 2H), 4.01-3.81 (m, 4H), 3.73-3.51 (m, 4H), 3.43-3.16 (m, 4H), 3.11-2.99 (m, 1H), 2.98-2.82 (m, 3H), 2.26-2.17 (m, 3H), 2.02-1.79 (m, 6H), 1.69-1.43 (m, 2H), 1.42-1.31 (m, 1H), 0.90 (s, 9H).

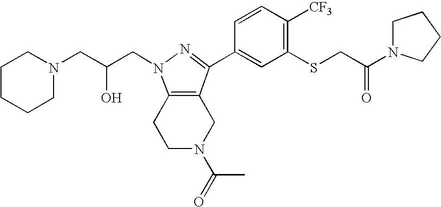

### Example 279

### 1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol

MS (ESI): mass calcd. for C₂₉H₃₈F₃N₅O₃S, 593.26; m/z found, 594.3 [M+H]⁺. ¹H NMR: 8.13 (s, 0.4H), 8.07 (s, 0.06H), 7.78 (d, J=8.3, 1H), 7.71 (t, J=8.4, 1H), 4.85-4.77 (m, 2H), 4.54-4.45 (m, 1H), 4.28-4.13 (m, 2H), 4.02-3.82 (m, 4H), 3.65-3.51 (m, 4H), 3.44-3.37 (m, 3H), 3.26-3.15 (m, 1H), 3.14-3.04 (m, 1H), 3.03-2.93 (m, 2H), 2.87 (t, J=5.7, 1H), 2.23 (d, J=9.3, 3H), 2.03-1.92 (m, 3H), 1.92-1.79 (m, 5H), 1.79-1.69 (m, 1H), 1.61-1.46 (m, 1H).

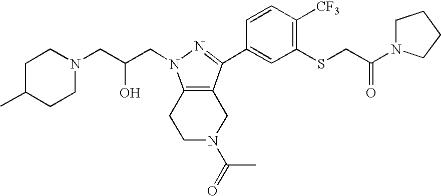

### Example 280

### 1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-methylpiperidin-1-yl)propan-2-ol

MS (ESI): mass calcd. for C₃₀H₄₀F₃N₅O₃S, 607.28; m/z found, 608.3 [M+H]⁺. ¹H NMR: 8.11 (s, 0.4H), 8.04 (s, 0.6H), 7.76 (d, J=8.3, 1H), 7.69 (t, J=7.7, 1H), 4.86-4.77 (m, 2H), 4.54-4.40 (m, 1H), 4.28-4.10 (m, 2H), 4.01-3.80 (m, 4H), 3.69-3.48 (m, 4H), 3.45-3.35 (m, 2H) 3.29-3.17 (m, 2H), 3.13-3.02 (m, 1H), 3.02-2.91 (m, 2H), 2.85 (t, J=5.6, 1H), 2.21 (d, J=7.6, 3H), 2.01-1.91 (m, 3H), 1.91-1.80 (m, 3H), 1.75-1.59 (m, 1H), 1.58-1.31 (m, 2H), 1.14-0.92 (m, 3H).

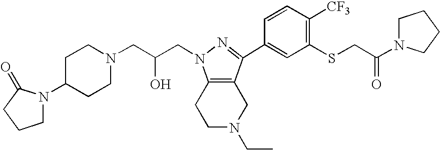

### Example 281

### 1-{1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}pyrrolidin-2-one

To a solution of 1-(2-hydroxy-3-{3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidine-4-carboxylic acid amide (16 mg, 0.025 mmol) in acetonitrile (0.30 mL) was added TEA (0.007 mL, 0.05 mmol), acetaldehyde (0.006 mL, 0.13 mmol) and NaCNBH₃ (4 mg, 0.05 mmol). The mixture was stirred under N₂ for 36 h, quenched with MeOH (1 mL), filtered, and concentrated. The residue was purified by reverse phase HPLC, yielding the title compound as a white powder (5 mg, 31%). MS (ESI): mass calcd. for C₃₃H₄₅F₃N₆O₃S, 662.32; m/z found, 663.4 [M+H]⁺. ¹H NMR: 8.12 (s, 1H), 7.77 (d, J=8.3, 1H), 7.68 (d, J=8.2, 1H), 4.85-4.74 (m, 1H), 4.59-4.48 (m, 2H), 4.34-4.18 (m, 2H), 4.17-4.07 (m, 1H), 3.90 (s, 3H), 3.80-3.67 (m, 2H), 3.59 (t, J=6.8, 2H), 3.53-3.35 (m, 8H), 3.29-3.23 (m, 3H), 3.22-3.09 (m, 2H), 2.39 (t, J=8.1, 2H), 2.21-2.10 (m, 1H), 2.08-2.01 (m, 3H), 2.00-1.92 (m, 4H), 1.91-1.83 (m, 3H), 1.48 (t, J=7.3, 3H).

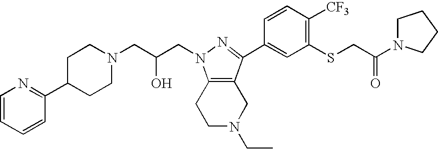

### Example 282

### 1-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol

This compound was prepared using methods similar to those described in Example 281 with the appropriate substituent changes. MS (ESI): mass calcd. for C₃₄H₄₃F₃N₆O₂S, 656.31; m/z found, 657.4 [M+H]⁺. ¹H NMR: 8.65-8.62 (m, 1H), 8.17-8.06 (m, 2H), 7.77 (d, J=8.3, 1H), 7.69 (d, J=8.6, 1H), 7.62 (d, J=8.0, 1H), 7.60-7.55 (m, 1H), 4.86-4.73 (m, 1H), 4.64-4.46 (m, 2H), 4.36-4.17 (m, 2H), 3.97-3.88 (m, 3H), 3.87-3.74 (m, 2H), 3.66-3.52 (m, 3H), 3.52-3.33 (m, 7H), 3.28-3.12 (m, 4H), 2.38-2.07 (m, 4H), 2.03-1.92 (m, 2H), 1.91-1.82 (m, 2H), 1.48 (t, J=7.3, 3H).

The compounds in Table 1 (Examples 283-896) were prepared using methods analogous to those described in the preceding examples.

TABLE 1

| EX# | Chemical Name | MS Found |
|---|---|---|
| 283 | 1-[3-{3-[2-(4-Fluoro-piperidin-1-yl)-ethylsulfanyl]-4-trifluoromethyl- | 628.3 |
| | phenyl}-1-(2-hydroxy-3-piperidin-1-yl-propyl)-1,4,6,7-tetrahydro- | |
| | pyrazolo[4,3-c]pyridin-5-yl]-2-hydroxy-ethanone | |
| 284 | (2S)-1-(4-Cyclohexyl-piperidin-1-yl)-3-(3-{3-[2-((3S)-fluoro-pyrrolidin-1- | 716.3 |
| | yl)-ethylsulfanyl]-4-trifluoromethyl-phenyl}-5-methanesulfonyl-4,5,6,7- | |
| | tetrahydro-pyrazolo[4,3-c]pyridin-1-yl)-propan-2-ol | |
| 285 | N-[1-(2-{5-[1-((2S)-Hydroxy-3-morpholin-4-yl-propyl)-5-methanesulfonyl- | 689.3 |
| | 4,5,6,7-tetrahydro-1H-pyrazolo[4,3-c]pyridin-3-yl]-2-trifluoromethyl- | |
| | phenylsulfanyl}-ethyl)-piperidin-4-yl]-acetamide | |
| 286 | (2S)-1-(3-{3-[2-((3S)-Fluoro-pyrrolidin-1-yl)-ethylsulfanyl]-4- | 711.2 |
| | trifluoromethyl-phenyl}-5-methanesulfonyl-4,5,6,7-tetrahydro- | |
| | pyrazolo[4,3-c]pyridin-1-yl)-3-(3′,4′,5′,6′-tetrahydro-2′H-[2,4′]bipyridinyl- | |
| | 1′-yl)-propan-2-ol | |
| 287 | (3S)-1-(2-{[5-{1-[(2S)-2-Hydroxy-3-morpholin-4-ylpropyl]-5- | 348.20 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)piperidin-3-ol | |
| 288 | 1-(3-{3-[(2-Piperidin-1-ylethyl)sulfanyl]-4-(trifluoromethyl)phenyl}-4,5,6,7- | 538.30 |
| | tetrahydro-1H-pyrazolo[4,3-c]pyridin-1-yl)-3-pyrrolidin-1-ylpropan-2-ol | |
| 289 | 1-Piperidin-1-yl-3-(3-{3-[(2-piperidin-1-ylethyl)sulfanyl]-4- | 552.29 |
| | (trifluoromethyl)phenyl}-4,5,6,7-tetrahydro-1H-pyrazolo[4,3-c]pyridin-1- | |
| | yl)propan-2-ol | |
| 290 | 1-[2-Hydroxy-3-(3-{3-[(2-piperidin-1-ylethyl)sulfanyl]-4- | 554.27 |
| | (trifluoromethyl)phenyl}-4,5,6,7-tetrahydro-1H-pyrazolo[4,3-c]pyridin-1- | |
| | yl)propyl]pyrrolidin-3-ol | |
| 291 | 1-(3-{3-[(2-Morpholin-4-ylethyl)sulfanyl]-4-(trifluoromethyl)phenyl}- | 554.30 |
| | 4,5,6,7-tetrahydro-1H-pyrazolo[4,3-c]pyridin-1-yl)-3-piperidin-1-ylpropan- | |
| | 2-ol | |
| 292 | 1-Morpholin-4-yl-3-(3-{3-[(2-piperidin-1-ylethyl)sulfanyl]-4- | 554.30 |
| | (trifluoromethyl)phenyl}-4,5,6,7-tetrahydro-1H-pyrazolo[4,3-c]pyridin-1- | |
| | yl)propan-2-ol | |
| 293 | 1-(4-Methylpiperidin-1-yl)-3-(3-{3-[(2-oxo-2-pyrrolidin-1-ylethyl)sulfanyl]- | 566.27 |
| | 4-(trifluoromethyl)phenyl}-4,5,6,7-tetrahydro-1H-pyrazolo[4,3-c]pyridin-1- | |
| | yl)propan-2-ol | |
| 294 | 1-[2-Hydroxy-3-(3-{3-[(2-oxo-2-pyrrolidin-1-ylethyl)sulfanyl]-4- | 568.30 |
| | (trifluoromethyl)phenyl}-4,5,6,7-tetrahydro-1H-pyrazolo[4,3-c]pyridin-1- | |
| | yl)propyl]piperidin-4-ol | |
| 295 | 1-[2-({5-[1-(2-Hydroxy-3-piperidin-1-ylpropyl)-4,5,6,7-tetrahydro-1H- | 568.40 |
| | pyrazolo[4,3-c]pyridin-3-yl]-2- | |
| | (trifluoromethyl)phenyl}sulfanyl)ethyl]piperidin-3-ol | |
| 296 | 1-(4-Fluoropiperidin-1-yl)-3-(3-{3-[(2-oxo-2-pyrrolidin-1-ylethyl)sulfanyl]- | 570.30 |
| | 4-(trifluoromethyl)phenyl}-4,5,6,7-tetrahydro-1H-pyrazolo[4,3-c]pyridin-1- | |
| | yl)propan-2-ol | |
| 297 | 1-{3-[3-{[2-(3,3-Difluoropyrrolidin-1-yl)ethyl]sulfanyl}-4- | 574.30 |
| | (trifluoromethyl)phenyl]-4,5,6,7-tetrahydro-1H-pyrazolo[4,3-c]pyridin-1- | |
| | yl}-3-piperidin-1-ylpropan-2-ol | |
| 298 | 1-{3-[3-{[2-(3,3-Difluoropyrrolidin-1-yl)ethyl]sulfanyl}-4- | 576.20 |
| | (trifluoromethyl)phenyl]-4,5,6,7-tetrahydro-1H-pyrazolo[4,3-c]pyridin-1- | |
| | yl}-3-morpholin-4-ylpropan-2-ol | |
| 299 | 1-(Hexahydrocyclopenta[c]pyrrol-2(1H)-yl)-3-(3-{3-[(2-oxo-2-pyrrolidin-1- | 578.30 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 300 | 1-(4,4-Dimethylpiperidin-1-yl)-3-(3-{3-[(2-oxo-2-pyrrolidin-1- | 580.28 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 301 | 1-[2-Hydroxy-3-(3-{3-[(2-oxo-2-pyrrolidin-1-ylethyl)sulfanyl]-4- | 582.30 |
| | (trifluoromethyl)phenyl}-4,5,6,7-tetrahydro-1H-pyrazolo[4,3-c]pyridin-1- | |
| | yl)propyl]-4-methylpiperidin-4-ol | |
| 302 | 1-[4-(Methyloxy)piperidin-1-yl]-3-(3-{3-[(2-oxo-2-pyrrolidin-1- | 582.30 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 303 | 1-{3-[3-{[2-(4-Fluoropiperidin-1-yl)ethyl]sulfanyl}-4- | 584.30 |
| | (trifluoromethyl)phenyl]-5-methyl-4,5,6,7-tetrahydro-1H-pyrazolo[4,3- | |
| | c]pyridin-1-yl}-3-piperidin-1-ylpropan-2-ol | |
| 304 | (2S)-1-{3-[3-{[2-(4-Fluoropiperidin-1-yl)ethyl]sulfanyl}-4- | 585.00 |
| | (trifluoromethyl)phenyl]-4,5,6,7-tetrahydro-1H-pyrazolo[4,3-c]pyridin-1- | |
| | yl}-3-(4-methylpiperidin-1-yl)propan-2-ol | |
| 305 | 1-(1,3-Dihydro-2H-isoindol-2-yl)-3-(3-{3-[(2-oxo-2-pyrrolidin-1- | 586.20 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 306 | 3-{3-[(2-Azetidin-1-ylethyl)sulfanyl]-4-(trifluoromethyl)phenyl}-5- | 586.20 |
| | (methylsulfonyl)-1-(3-piperidin-1-ylpropyl)-4,5,6,7-tetrahydro-1H- | |
| | pyrazolo[4,3-c]pyridine | |
| 307 | 1-[2-Hydroxy-3-(3-{3-[(2-oxo-2-pyrrolidin-1-ylethyl)sulfanyl]-4- | 595.26 |
| | (trifluoromethyl)phenyl}-4,5,6,7-tetrahydro-1H-pyrazolo[4,3-c]pyridin-1- | |
| | yl)propyl]piperidine-4-carboxamide | |
| 308 | 1-(4-Acetylpiperazin-1-yl)-3-(3-{3-[(2-piperidin-1-ylethyl)sulfanyl]-4- | 595.30 |
| | (trifluoromethyl)phenyl}-4,5,6,7-tetrahydro-1H-pyrazolo[4,3-c]pyridin-1- | |
| | yl)propan-2-ol | |
| 309 | 1-[2-Hydroxy-3-(3-{3-[(2-piperidin-1-ylethyl)sulfanyl]-4- | 595.30 |
| | (trifluoromethyl)phenyl}-4,5,6,7-tetrahydro-1H-pyrazolo[4,3-c]pyridin-1- | |
| | yl)propyl]piperidine-4-carboxamide | |
| 310 | 1-(3,3-Difluoropyrrolidin-1-yl)-3-{3-[3-{[2-(3,3-difluoropyrrolidin-1- | 596.20 |
| | yl)ethyl]sulfanyl}-4-(trifluoromethyl)phenyl]-4,5,6,7-tetrahydro-1H- | |
| | pyrazolo[4,3-c]pyridin-1-yl}propan-2-ol | |
| 311 | (2S)-1-(4,4-Dimethylpiperidin-1-yl)-3-{3-[3-{[2-(4-fluoropiperidin-1- | 599.00 |
| | yl)ethyl]sulfanyl}-4-(trifluoromethyl)phenyl]-4,5,6,7-tetrahydro-1H- | |
| | pyrazolo[4,3-c]pyridin-1-yl}propan-2-ol | |
| 312 | 5-(Methylsulfonyl)-1-(3-piperidin-1-ylpropyl)-3-{3-[(2-pyrrolidin-1- | 600.26 |
| | ylethyl)sulfanyl]-4-(trifluoromethyl)phenyl}-4,5,6,7-tetrahydro-1H- | |
| | pyrazolo[4,3-c]pyridine | |
| 313 | 1-(3,4-Dihydroisoquinolin-2(1H)-yl)-3-(3-{3-[(2-oxo-2-pyrrolidin-1- | 600.30 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 314 | N-Methyl-N-[2-({5-[5-(methylsulfonyl)-1-(3-piperidin-1-ylpropyl)-4,5,6,7- | 602.30 |
| | tetrahydro-1H-pyrazolo[4,3-c]pyridin-3-yl]-2- | |
| | (trifluoromethyl)phenyl}sulfanyl)ethyl]propan-2-amine | |
| 315 | 1-(3-{3-[(2-Oxo-2-pyrrolidin-1-ylethyl)sulfanyl]-4-(trifluoromethyl)phenyl}- | 606.20 |
| | 4,5,6,7-tetrahydro-1H-pyrazolo[4,3-c]pyridin-1-yl)-3-[(3S)-3- | |
| | (trifluoromethyl)pyrrolidin-1-yl]propan-2-ol | |
| 316 | 1-(Octahydroisoquinolin-2(1H)-yl)-3-(3-{3-[(2-oxo-2-pyrrolidin-1- | 606.30 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 317 | 2-(1-[(2S)-2-Hydroxy-3-piperidin-1-ylpropyl]-3-{3-[(2-pyrrolidin-1- | 609.60 |
| | ylethyl)sulfanyl]-4-(trifluoromethyl)phenyl}-1,4,6,7-tetrahydro-5H- | |
| | pyrazolo[4,3-c]pyridin-5-yl)-2-oxoacetamide | |
| 318 | 1-(1,4-Dioxa-8-azaspiro[4.5]dec-8-yl)-3-(3-{3-[(2-oxo-2-pyrrolidin-1- | 610.26 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 319 | 1-[4-(1-Hydroxy-1-methylethyl)piperidin-1-yl]-3-(3-{3-[(2-oxo-2-pyrrolidin- | 610.30 |
| | 1-ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 320 | 1-{1-[2-Hydroxy-3-(3-{3-[(2-piperidin-1-ylethyl)sulfanyl]-4- | 610.31 |
| | (trifluoromethyl)phenyl}-4,5,6,7-tetrahydro-1H-pyrazolo[4,3-c]pyridin-1- | |
| | yl)propyl]piperidin-4-yl}urea | |
| 321 | (2S)-1-{5-Acetyl-3-[3-{[2-(4-fluoropiperidin-1-yl)ethyl]sulfanyl}-4- | 612.29 |
| | (trifluoromethyl)phenyl]-4,5,6,7-tetrahydro-1H-pyrazolo[4,3-c]pyridin-1- | |
| | yl}-3-piperidin-1-ylpropan-2-ol | |
| 322 | 3-[3-{[2-(4-Fluoropiperidin-1-yl)ethyl]sulfanyl}-4-(trifluoromethyl)phenyl]- | 613.29 |
| | 1-[(2S)-2-hydroxy-3-piperidin-1-ylpropyl]-1,4,6,7-tetrahydro-5H- | |
| | pyrazolo[4,3-c]pyridine-5-carboxamide | |
| 323 | 1-[(2S)-3-{3-[3-{[2-(4-Fluoropiperidin-1-yl)ethyl]sulfanyl}-4- | 614.00 |
| | (trifluoromethyl)phenyl]-4,5,6,7-tetrahydro-1H-pyrazolo[4,3-c]pyridin-1- | |
| | yl}-2-hydroxypropyl]piperidine-4-carboxamide | |
| 324 | 1-[2-({5-[5-(Methylsulfonyl)-1-(3-piperidin-1-ylpropyl)-4,5,6,7-tetrahydro- | 615.20 |
| | 1H-pyrazolo[4,3-c]pyridin-3-yl]-2- | |
| | (trifluoromethyl)phenyl}sulfanyl)ethyl]imidazolidin-4-one | |
| 325 | 1-{3-[3-{[2-(Cyclopentylamino)ethyl]sulfanyl}-4-(trifluoromethyl)phenyl]-5- | 616.25 |
| | (methylsulfonyl)-4,5,6,7-tetrahydro-1H-pyrazolo[4,3-c]pyridin-1-yl}-3- | |
| | pyrrolidin-1-ylpropan-2-ol | |
| 326 | 5-(Methylsulfonyl)-3-{3-[(2-morpholin-4-ylethyl)sulfanyl]-4- | 616.25 |
| | (trifluoromethyl)phenyl}-1-(3-piperidin-1-ylpropyl)-4,5,6,7-tetrahydro-1H- | |
| | pyrazolo[4,3-c]pyridine | |
| 327 | 1-(3-{3-[3-{[2-(3,3-Difluoropyrrolidin-1-yl)ethyl]sulfanyl}-4- | 617.30 |
| | (trifluoromethyl)phenyl]-4,5,6,7-tetrahydro-1H-pyrazolo[4,3-c]pyridin-1- | |
| | yl}-2-hydroxypropyl)piperidine-4-carboxamide | |
| 328 | 1-[5-(Methylsulfonyl)-3-{3-[(2-morpholin-4-ylethyl)sulfanyl]-4- | 618.20 |
| | (trifluoromethyl)phenyl}-4,5,6,7-tetrahydro-1H-pyrazolo[4,3-c]pyridin-1- | |
| | yl]-3-pyrrolidin-1-ylpropan-2-ol | |
| 329 | 1-(5-Acetyl-3-{3-[(2-oxo-2-pyrrolidin-1-ylethyl)sulfanyl]-4- | 620.22 |
| | (trifluoromethyl)phenyl}-4,5,6,7-tetrahydro-1H-pyrazolo[4,3-c]pyridin-1- | |
| | yl)-3-(hexahydrocyclopenta[c]pyrrol-2(1H)-yl)propan-2-ol | |
| 330 | 1-(5-Acetyl-3-{3-[(2-oxo-2-pyrrolidin-1-ylethyl)sulfanyl]-4- | 622.30 |
| | (trifluoromethyl)phenyl}-4,5,6,7-tetrahydro-1H-pyrazolo[4,3-c]pyridin-1- | |
| | yl)-3-(4,4-dimethylpiperidin-1-yl)propan-2-ol | |
| 331 | 1-[3-(5-Ethyl-3-{3-[(2-oxo-2-pyrrolidin-1-ylethyl)sulfanyl]-4- | 623.29 |
| | (trifluoromethyl)phenyl}-4,5,6,7-tetrahydro-1H-pyrazolo[4,3-c]pyridin-1- | |
| | yl)-2-hydroxypropyl]piperidine-4-carboxamide | |
| 332 | 2-Hydroxy-N-{1-[2-hydroxy-3-(3-{3-[(2-oxo-2-pyrrolidin-1- | 625.27 |
| | ylethyl)sulfanyl]-4-(trifluoromethyl)phenyl}-4,5,6,7-tetrahydro-1H- | |
| | pyrazolo[4,3-c]pyridin-1-yl)propyl]piperidin-4-yl}acetamide | |
| 333 | 2-Hydroxy-N-{1-[2-hydroxy-3-(3-{3-[(2-piperidin-1-ylethyl)sulfanyl]-4- | 625.31 |
| | (trifluoromethyl)phenyl}-4,5,6,7-tetrahydro-1H-pyrazolo[4,3-c]pyridin-1- | |
| | yl)propyl]piperidin-4-yl}acetamide | |
| 334 | (2S)-1-[1-(2-Hydroxy-3-piperidin-1-ylpropyl)-3-{3-[(2-morpholin-4- | 626.30 |
| | ylethyl)sulfanyl]-4-(trifluoromethyl)phenyl}-1,4,6,7-tetrahydro-5H- | |
| | pyrazolo[4,3-c]pyridin-5-yl]-1-oxopropan-2-ol | |
| 335 | 1-(5-[(Methyloxy)acetyl]-3-{3-[(2-morpholin-4-ylethyl)sulfanyl]-4- | 626.30 |
| | (trifluoromethyl)phenyl}-4,5,6,7-tetrahydro-1H-pyrazolo[4,3-c]pyridin-1- | |
| | yl)-3-piperidin-1-ylpropan-2-ol | |
| 336 | 1-[2-({5-[5-(Hydroxyacetyl)-1-(2-hydroxy-3-piperidin-1-ylpropyl)-4,5,6,7- | 626.30 |
| | tetrahydro-1H-pyrazolo[4,3-c]pyridin-3-yl]-2- | |
| | (trifluoromethyl)phenyl}sulfanyl)ethyl]piperidin-3-ol | |
| 337 | (2S)-1-{3-[3-{[2-(4-Fluoropiperidin-1-yl)ethyl]sulfanyl}-4- | 626.31 |
| | (trifluoromethyl)phenyl]-5-propanoyl-4,5,6,7-tetrahydro-1H-pyrazolo[4,3- | |
| | c]pyridin-1-yl}-3-piperidin-1-ylpropan-2-ol | |
| 338 | 1-{3-[3-{[2-(4-Fluoropiperidin-1-yl)ethyl]sulfanyl}-4- | 627.30 |
| | (trifluoromethyl)phenyl]-5-glycyl-4,5,6,7-tetrahydro-1H-pyrazolo[4,3- | |
| | c]pyridin-1-yl}-3-piperidin-1-ylpropan-2-ol | |
| 339 | 1-(5-Acetyl-3-{3-[(2-oxo-2-pyrrolidin-1-ylethyl)sulfanyl]-4- | 628.20 |
| | (trifluoromethyl)phenyl}-4,5,6,7-tetrahydro-1H-pyrazolo[4,3-c]pyridin-1- | |
| | yl)-3-(1,3-dihydro-2H-isoindol-2-yl)propan-2-ol | |
| 340 | N-[2-({5-[5-(Methylsulfonyl)-1-(3-piperidin-1-ylpropyl)-4,5,6,7-tetrahydro- | 628.29 |
| | 1H-pyrazolo[4,3-c]pyridin-3-yl]-2- | |
| | (trifluoromethyl)phenyl}sulfanyl)ethyl]cyclohexanamine | |
| 341 | 1-{1-[(2S)-3-{3-[3-{[2-(4-Fluoropiperidin-1-yl)ethyl]sulfanyl}-4- | 629.10 |
| | (trifluoromethyl)phenyl]-4,5,6,7-tetrahydro-1H-pyrazolo[4,3-c]pyridin-1- | |
| | yl}-2-hydroxypropyl]piperidin-4-yl}urea | |
| 342 | N-(2-{[5-{1-[3-(4-Cyclopropylpiperazin-1-yl)propyl]-5-(methylsulfonyl)- | 629.30 |
| | 4,5,6,7-tetrahydro-1H-pyrazolo[4,3-c]pyridin-3-yl}-2- | |
| | (trifluoromethyl)phenyl]sulfanyl}ethyl)propan-1-amine | |
| 343 | 1-(3-{3-[(2-Piperidin-1-ylethyl)sulfanyl]-4-(trifluoromethyl)phenyl}-4,5,6,7- | 629.32 |
| | tetrahydro-1H-pyrazolo[4,3-c]pyridin-1-yl)-3-(4-pyridin-4-ylpiperidin-1- | |
| | yl)propan-2-ol | |
| 344 | (2S)-1-{3-[3-{[2-(4-Methylpiperidin-1-yl)ethyl]sulfanyl}-4- | 630.27 |
| | (trifluoromethyl)phenyl]-5-(methylsulfonyl)-4,5,6,7-tetrahydro-1H- | |
| | pyrazolo[4,3-c]pyridin-1-yl}-3-pyrrolidin-1-ylpropan-2-ol | |
| 345 | 1-(4-Methylpiperidin-1-yl)-3-[5-(methylsulfonyl)-3-{3-[(2-pyrrolidin-1- | 630.27 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 346 | 1-{3-[3-{[2-(Cyclopentylamino)ethyl]sulfanyl}-4-(trifluoromethyl)phenyl]-5- | 630.27 |
| | (methylsulfonyl)-4,5,6,7-tetrahydro-1H-pyrazolo[4,3-c]pyridin-1-yl}-3- | |
| | piperidin-1-ylpropan-2-ol | |
| 347 | (2S)-1-{3-[3-{[2-(5,6-Dihydropyrimidin-1(4H)-yl)ethyl]sulfanyl}-4- | 631.20 |
| | (trifluoromethyl)phenyl]-5-(methylsulfonyl)-4,5,6,7-tetrahydro-1H- | |
| | pyrazolo[4,3-c]pyridin-1-yl}-3-morpholin-4-ylpropan-2-ol | |
| 348 | 1-(4-Methylpiperazin-1-yl)-3-[5-(methylsulfonyl)-3-{3-[(2-pyrrolidin-1- | 631.26 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 349 | 1-{2-Hydroxy-3-[5-(methylsulfonyl)-3-{3-[(2-piperidin-1-ylethyl)sulfanyl]- | 632.20 |
| | 4-(trifluoromethyl)phenyl}-4,5,6,7-tetrahydro-1H-pyrazolo[4,3-c]pyridin-1- | |
| | yl]propyl}pyrrolidin-3-ol | |
| 350 | 1-[(3S)-3-(Hydroxymethyl)pyrrolidin-1-yl]-3-[5-(methylsulfonyl)-3-{3-[(2- | 632.25 |
| | pyrrolidin-1-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 351 | 1-[(3S)-3-Methylmorpholin-4-yl]-3-[5-(methylsulfonyl)-3-{3-[(2-pyrrolidin- | 632.25 |
| | 1-ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 352 | 1-[1-(3-{3-[3-{[2-(3,3-Difluoropyrrolidin-1-yl)ethyl]sulfanyl}-4- | 632.30 |
| | (trifluoromethyl)phenyl]-4,5,6,7-tetrahydro-1H-pyrazolo[4,3-c]pyridin-1- | |
| | yl}-2-hydroxypropyl)piperidin-4-yl]urea | |
| 353 | (2S)-1-(5,6-Dihydropyrimidin-1(4H)-yl)-3-{3-[3-({2-[(3S)-3- | 633.20 |
| | fluoropyrrolidin-1-yl]ethyl}sulfanyl)-4-(trifluoromethyl)phenyl]-5- | |
| | (methylsulfonyl)-4,5,6,7-tetrahydro-1H-pyrazolo[4,3-c]pyridin-1- | |
| | yl}propan-2-ol | |
| 354 | (2S)-1-{3-[3-({2-[(3S)-3-Fluoropyrrolidin-1-yl]ethyl}sulfanyl)-4- | 634.20 |
| | (trifluoromethyl)phenyl]-5-(methylsulfonyl)-4,5,6,7-tetrahydro-1H- | |
| | pyrazolo[4,3-c]pyridin-1-yl}-3-piperidin-1-ylpropan-2-ol | |
| 355 | 1-{3-[3-{[2-(3,3-Difluoropyrrolidin-1-yl)ethyl]sulfanyl}-4- | 634.20 |
| | (trifluoromethyl)phenyl]-5-(hydroxyacetyl)-4,5,6,7-tetrahydro-1H- | |
| | pyrazolo[4,3-c]pyridin-1-yl}-3-morpholin-4-ylpropan-2-ol | |
| 356 | 1-{2-Hydroxy-3-[5-(methylsulfonyl)-3-{3-[(2-morpholin-4-ylethyl)sulfanyl]- | 634.20 |
| | 4-(trifluoromethyl)phenyl}-4,5,6,7-tetrahydro-1H-pyrazolo[4,3-c]pyridin-1- | |
| | yl]propyl}pyrrolidin-3-ol | |
| 357 | 5-{1-[2-Hydroxy-3-(3-{3-[(2-piperidin-1-ylethyl)sulfanyl]-4- | 635.30 |
| | (trifluoromethyl)phenyl}-4,5,6,7-tetrahydro-1H-pyrazolo[4,3-c]pyridin-1- | |
| | yl)propyl]piperidin-4-yl}-2,4-dihydro-3H-1,2,4-triazol-3-one | |
| 358 | (2S)-1-{3-[3-({2-[(3S)-3-Fluoropyrrolidin-1-yl]ethyl}sulfanyl)-4- | 636.20 |
| | (trifluoromethyl)phenyl]-5-(methylsulfonyl)-4,5,6,7-tetrahydro-1H- | |
| | pyrazolo[4,3-c]pyridin-1-yl}-3-morpholin-4-ylpropan-2-ol | |
| 359 | 2-{1-[(2S)-2-Hydroxy-3-piperidin-1-ylpropyl]-3-[3-{[2-(4-methylpiperidin- | 637.30 |
| | 1-yl)ethyl]sulfanyl}-4-(trifluoromethyl)phenyl]-1,4,6,7-tetrahydro-5H- | |
| | pyrazolo[4,3-c]pyridin-5-yl}-2-oxoacetamide | |
| 360 | 1-(3-{3-[(2-Morpholin-4-yl-2-oxoethyl)sulfanyl]-4-(trifluoromethyl)phenyl}- | 637.30 |
| | 4,5,6,7-tetrahydro-1H-pyrazolo[4,3-c]pyridin-1-yl)-3-(4-pyrrolidin-1- | |
| | ylpiperidin-1-yl)propan-2-ol | |
| 361 | 1-(4-Morpholin-4-ylpiperidin-1-yl)-3-(3-{3-[(2-piperidin-1-ylethyl)sulfanyl]- | 637.30 |
| | 4-(trifluoromethyl)phenyl}-4,5,6,7-tetrahydro-1H-pyrazolo[4,3-c]pyridin-1- | |
| | yl)propan-2-ol | |
| 362 | 2-{1-(2-Hydroxy-3-piperidin-1-ylpropyl)-3-[3-{[2-(4-methylpiperidin-1- | 637.31 |
| | yl)ethyl]sulfanyl}-4-(trifluoromethyl)phenyl]-1,4,6,7-tetrahydro-5H- | |
| | pyrazolo[4,3-c]pyridin-5-yl}-2-oxoacetamide | |
| 363 | N-{1-[(2S)-3-(5-Acetyl-3-{3-[(2-pyrrolidin-1-ylethyl)sulfanyl]-4- | 637.70 |
| | (trifluoromethyl)phenyl}-4,5,6,7-tetrahydro-1H-pyrazolo[4,3-c]pyridin-1- | |
| | yl)-2-hydroxypropyl]piperidin-4-yl}acetamide | |
| 364 | (4R)-1-{1-[(2S)-3-(5-Acetyl-3-{3-[(2-pyrrolidin-1-ylethyl)sulfanyl]-4- | 637.70 |
| | (trifluoromethyl)phenyl}-4,5,6,7-tetrahydro-1H-pyrazolo[4,3-c]pyridin-1- | |
| | yl)-2-hydroxypropyl]piperidin-4-yl}-4-hydroxypyrrolidin-2-one | |
| 365 | Ethyl {1-[2-hydroxy-3-(3-{3-[(2-piperidin-1-ylethyl)sulfanyl]-4- | 639.32 |
| | (trifluoromethyl)phenyl}-4,5,6,7-tetrahydro-1H-pyrazolo[4,3-c]pyridin-1- | |
| | yl)propyl]piperidin-4-yl}carbamate | |
| 366 | N-[2-({5-[1-(2-Hydroxy-3-piperidin-1-ylpropyl)-5-(methylsulfonyl)-4,5,6,7- | 640.20 |
| | tetrahydro-1H-pyrazolo[4,3-c]pyridin-3-yl]-2- | |
| | (trifluoromethyl)phenyl}sulfanyl)ethyl]methanesulfonamide | |
| 367 | 3-[3-{[2-(4-Fluoropiperidin-1-yl)ethyl]sulfanyl}-4-(trifluoromethyl)phenyl]- | 641.32 |
| | 1-[(2S)-2-hydroxy-3-piperidin-1-ylpropyl]-N,N-dimethyl-1,4,6,7- | |
| | tetrahydro-5H-pyrazolo[4,3-c]pyridine-5-carboxamide | |
| 368 | 1-(5-[(Methylsulfanyl)acetyl]-3-{3-[(2-morpholin-4-ylethyl)sulfanyl]-4- | 642.30 |
| | (trifluoromethyl)phenyl}-4,5,6,7-tetrahydro-1H-pyrazolo[4,3-c]pyridin-1- | |
| | yl)-3-piperidin-1-ylpropan-2-ol | |
| 369 | 1-(5-Acetyl-3-{3-[(2-oxo-2-pyrrolidin-1-ylethyl)sulfanyl]-4- | 642.30 |
| | (trifluoromethyl)phenyl}-4,5,6,7-tetrahydro-1H-pyrazolo[4,3-c]pyridin-1- | |
| | yl)-3-(3,4-dihydroisoquinolin-2(1H)-yl)propan-2-ol | |
| 370 | (2S)-1-{3-[3-{[2-(4-Fluoropiperidin-1-yl)ethyl]sulfanyl}-4- | 642.30 |
| | (trifluoromethyl)phenyl]-1-(2-hydroxy-3-piperidin-1-ylpropyl)-1,4,6,7- | |
| | tetrahydro-5H-pyrazolo[4,3-c]pyridin-5-yl}-1-oxopropan-2-ol | |
| 371 | (2S)-1-{3-[3-{[2-(4-Fluoropiperidin-1-yl)ethyl]sulfanyl}-4- | 642.30 |
| | (trifluoromethyl)phenyl]-1-[(2S)-2-hydroxy-3-piperidin-1-ylpropyl]-1,4,6,7- | |
| | tetrahydro-5H-pyrazolo[4,3-c]pyridin-5-yl}-1-oxopropan-2-ol | |
| 372 | 1-{3-[3-{[2-(4-Fluoropiperidin-1-yl)ethyl]sulfanyl}-4- | 642.30 |
| | (trifluoromethyl)phenyl]-5-[(methyloxy)acetyl]-4,5,6,7-tetrahydro-1H- | |
| | pyrazolo[4,3-c]pyridin-1-yl}-3-piperidin-1-ylpropan-2-ol | |
| 373 | (2S)-1-{3-[3-{[2-(4-Fluoropiperidin-1-yl)ethyl]sulfanyl}-4- | 642.30 |
| | (trifluoromethyl)phenyl]-5-[(methyloxy)acetyl]-4,5,6,7-tetrahydro-1H- | |
| | pyrazolo[4,3-c]pyridin-1-yl}-3-piperidin-1-ylpropan-2-ol | |
| 374 | 3-Methyl-4-[2-({5-[5-(methylsulfonyl)-1-(3-piperidin-1-ylpropyl)-4,5,6,7- | 643.30 |
| | tetrahydro-1H-pyrazolo[4,3-c]pyridin-3-yl]-2- | |
| | (trifluoromethyl)phenyl}sulfanyl)ethyl]piperazin-2-one | |
| 375 | N-{1-[(2S)-3-{3-[3-{[2-(4-Fluoropiperidin-1-yl)ethyl]sulfanyl}-4- | 644.00 |
| | (trifluoromethyl)phenyl]-4,5,6,7-tetrahydro-1H-pyrazolo[4,3-c]pyridin-1- | |
| | yl}-2-hydroxypropyl]piperidin-4-yl}-2-hydroxyacetamide | |
| 376 | 1-[5-(Methylsulfonyl)-3-{3-[(2-oxo-2-piperidin-1-ylethyl)sulfanyl]-4- | 644.25 |
| | (trifluoromethyl)phenyl}-4,5,6,7-tetrahydro-1H-pyrazolo[4,3-c]pyridin-1- | |
| | yl]-3-piperidin-1-ylpropan-2-ol | |
| 377 | (2S)-1-{3-[3-{[2-(4-Methylpiperidin-1-yl)ethyl]sulfanyl}-4- | 644.28 |
| | (trifluoromethyl)phenyl]-5-(methylsulfonyl)-4,5,6,7-tetrahydro-1H- | |
| | pyrazolo[4,3-c]pyridin-1-yl}-3-piperidin-1-ylpropan-2-ol | |
| 378 | 1-(4,4-Dimethylpiperidin-1-yl)-3-[5-(methylsulfonyl)-3-{3-[(2-pyrrolidin-1- | 644.28 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 379 | N-{1-[2-Hydroxy-3-(3-{3-[(2-oxo-2-pyrrolidin-1-ylethyl)sulfanyl]-4- | 645.24 |
| | (trifluoromethyl)phenyl}-4,5,6,7-tetrahydro-1H-pyrazolo[4,3-c]pyridin-1- | |
| | yl)propyl]piperidin-4-yl}methanesulfonamide | |
| 380 | 2-{4-[2-Hydroxy-3-(3-{3-[(2-oxo-2-pyrrolidin-1-ylethyl)sulfanyl]-4- | 645.28 |
| | (trifluoromethyl)phenyl}-4,5,6,7-tetrahydro-1H-pyrazolo[4,3-c]pyridin-1- | |
| | yl)propyl]piperazin-1-yl}phenol | |
| 381 | N-{1-[2-Hydroxy-3-(3-{3-[(2-piperidin-1-ylethyl)sulfanyl]-4- | 645.28 |
| | (trifluoromethyl)phenyl}-4,5,6,7-tetrahydro-1H-pyrazolo[4,3-c]pyridin-1- | |
| | yl)propyl]piperidin-4-yl}methanesulfonamide | |
| 382 | 1-(4-Methyl-1,4-diazepan-1-yl)-3-[5-(methylsulfonyl)-3-{3-[(2-pyrrolidin-1- | 645.28 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 383 | 3-{4-[2-Hydroxy-3-(3-{3-[(2-piperidin-1-ylethyl)sulfanyl]-4- | 645.31 |
| | (trifluoromethyl)phenyl}-4,5,6,7-tetrahydro-1H-pyrazolo[4,3-c]pyridin-1- | |
| | yl)propyl]piperazin-1-yl}phenol | |
| 384 | 4-{4-[2-Hydroxy-3-(3-{3-[(2-piperidin-1-ylethyl)sulfanyl]-4- | 645.31 |
| | (trifluoromethyl)phenyl}-4,5,6,7-tetrahydro-1H-pyrazolo[4,3-c]pyridin-1- | |
| | yl)propyl]piperazin-1-yl}phenol | |
| 385 | 2-{4-[2-Hydroxy-3-(3-{3-[(2-piperidin-1-ylethyl)sulfanyl]-4- | 645.31 |
| | (trifluoromethyl)phenyl}-4,5,6,7-tetrahydro-1H-pyrazolo[4,3-c]pyridin-1- | |
| | yl)propyl]piperazin-1-yl}phenol | |
| 386 | 1-{2-Hydroxy-3-[5-(methylsulfonyl)-3-{3-[(2-pyrrolidin-1-ylethyl)sulfanyl]- | 646.20 |
| | 4-(trifluoromethyl)phenyl}-4,5,6,7-tetrahydro-1H-pyrazolo[4,3-c]pyridin-1- | |
| | yl]propyl}-4-methylpiperidin-4-ol | |
| 387 | (2S)-1-(3,6-Dihydropyridin-1(2H)-yl)-3-{3-[3-{[2-(3-fluoropiperidin-1- | 646.20 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 388 | 1-{3-[3-({2-[3-(Hydroxymethyl)piperidin-1-yl]ethyl}sulfanyl)-4- | 646.20 |
| | (trifluoromethyl)phenyl]-5-(methylsulfonyl)-4,5,6,7-tetrahydro-1H- | |
| | pyrazolo[4,3-c]pyridin-1-yl}-3-pyrrolidin-1-ylpropan-2-ol | |
| 389 | (3S)-1-[({5-[1-(2-Hydroxy-3-piperidin-1-ylpropyl)-5-(methylsulfonyl)- | 646.23 |
| | 4,5,6,7-tetrahydro-1H-pyrazolo[4,3-c]pyridin-3-yl]-2- | |
| | (trifluoromethyl)phenyl}sulfanyl)acetyl]pyrrolidin-3-ol | |
| 390 | (3R)-1-[({5-[1-(2-Hydroxy-3-piperidin-1-ylpropyl)-5-(methylsulfonyl)- | 646.23 |
| | 4,5,6,7-tetrahydro-1H-pyrazolo[4,3-c]pyridin-3-yl]-2- | |
| | (trifluoromethyl)phenyl}sulfanyl)acetyl]pyrrolidin-3-ol | |
| 391 | (2S)-1-{3-[3-{[2-(4-Methylpiperidin-1-yl)ethyl]sulfanyl}-4- | 646.26 |
| | (trifluoromethyl)phenyl]-5-(methylsulfonyl)-4,5,6,7-tetrahydro-1H- | |
| | pyrazolo[4,3-c]pyridin-1-yl}-3-morpholin-4-ylpropan-2-ol | |
| 392 | (2S)-1-{3-[3-({2-[(3S)-3-Methylmorpholin-4-yl]ethyl}sulfanyl)-4- | 646.30 |
| | (trifluoromethyl)phenyl]-5-(methylsulfonyl)-4,5,6,7-tetrahydro-1H- | |
| | pyrazolo[4,3-c]pyridin-1-yl}-3-piperidin-1-ylpropan-2-ol | |
| 393 | (3S)-1-(2-{[5-{1-[(2S)-2-Hydroxy-3-piperidin-1-ylpropyl]-5- | 646.30 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)piperidin-3-ol | |
| 394 | (2S)-1-(5,6-Dihydropyrimidin-1(4H)-yl)-3-{3-[3-{[2-(4-fluoropiperidin-1- | 647.20 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 395 | (2S)-1-(5,6-Dihydropyrimidin-1(4H)-yl)-3-{3-[3-{[2-(3-fluoropiperidin-1- | 647.20 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 396 | 1-(4-Methylpiperazin-1-yl)-3-[5-(methylsulfonyl)-3-{3-[(2-morpholin-4- | 647.20 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 397 | 1-[4-(4-Fluorophenyl)piperazin-1-yl]-3-(3-{3-[(2-oxo-2-pyrrolidin-1- | 647.27 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 398 | N-[1-(3-{3-[3-{[2-(3,3-Difluoropyrrolidin-1-yl)ethyl]sulfanyl}-4- | 647.30 |
| | (trifluoromethyl)phenyl]-4,5,6,7-tetrahydro-1H-pyrazolo[4,3-c]pyridin-1- | |
| | yl}-2-hydroxypropyl)piperidin-4-yl]-2-hydroxyacetamide | |
| 399 | 1-{3-[3-{[2-(4-Fluoropiperidin-1-yl)ethyl]sulfanyl}-4- | 647.30 |
| | (trifluoromethyl)phenyl]-4,5,6,7-tetrahydro-1H-pyrazolo[4,3-c]pyridin-1- | |
| | yl}-3-(4-phenylpiperazin-1-yl)propan-2-ol | |
| 400 | (2S)-1-{3-[3-{[2-(4-Fluoropiperidin-1-yl)ethyl]sulfanyl}-4- | 647.31 |
| | (trifluoromethyl)phenyl]-4,5,6,7-tetrahydro-1H-pyrazolo[4,3-c]pyridin-1- | |
| | yl}-3-(4-pyridin-4-ylpiperidin-1-yl)propan-2-ol | |
| 401 | 1-[4-(2-Fluorophenyl)piperazin-1-yl]-3-(3-{3-[(2-piperidin-1- | 647.31 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 402 | (2S)-1-{3-[3-{[2-(4-Fluoropiperidin-1-yl)ethyl]sulfanyl}-4- | 647.31 |
| | (trifluoromethyl)phenyl]-4,5,6,7-tetrahydro-1H-pyrazolo[4,3-c]pyridin-1- | |
| | yl}-3-(4-phenylpiperazin-1-yl)propan-2-ol | |
| 403 | 1-[4-(4-Fluorophenyl)piperazin-1-yl]-3-(3-{3-[(2-piperidin-1- | 647.31 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 404 | (2S)-1-Azepan-1-yl-3-{3-[3-({2-[(3S)-3-fluoropyrrolidin-1- | 648.20 |
| | yl]ethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 405 | (3R)-1-{2-Hydroxy-3-[5-(methylsulfonyl)-3-{3-[(2-morpholin-4- | 648.20 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-3-ol | |
| 406 | 1-{2-Hydroxy-3-[5-(methylsulfonyl)-3-{3-[(2-morpholin-4-ylethyl)sulfanyl]- | 648.20 |
| | 4-(trifluoromethyl)phenyl}-4,5,6,7-tetrahydro-1H-pyrazolo[4,3-c]pyridin-1- | |
| | yl]propyl}piperidin-4-ol | |
| 407 | (3S)-1-{2-Hydroxy-3-[5-(methylsulfonyl)-3-{3-[(2-morpholin-4-yl-2- | 648.21 |
| | oxoethyl)sulfanyl]-4-(trifluoromethyl)phenyl}-4,5,6,7-tetrahydro-1H- | |
| | pyrazolo[4,3-c]pyridin-1-yl]propyl}pyrrolidin-3-ol | |
| 408 | (3R)-1-{2-Hydroxy-3-[5-(methylsulfonyl)-3-{3-[(2-morpholin-4-yl-2- | 648.21 |
| | oxoethyl)sulfanyl]-4-(trifluoromethyl)phenyl}-4,5,6,7-tetrahydro-1H- | |
| | pyrazolo[4,3-c]pyridin-1-yl]propyl}pyrrolidin-3-ol | |
| 409 | (2S)-1-{3-[3-({2-[(3S)-3-Fluoropyrrolidin-1-yl]ethyl}sulfanyl)-4- | 648.30 |
| | (trifluoromethyl)phenyl]-5-(methylsulfonyl)-4,5,6,7-tetrahydro-1H- | |
| | pyrazolo[4,3-c]pyridin-1-yl}-3-(4-methylpiperidin-1-yl)propan-2-ol | |
| 410 | 1-(3-{3-[(2-Oxo-2-pyrrolidin-1-ylethyl)sulfanyl]-4-(trifluoromethyl)phenyl}- | 649.31 |
| | 4,5,6,7-tetrahydro-1H-pyrazolo[4,3-c]pyridin-1-yl)-3-[4-(pyrrolidin-1- | |
| | ylcarbonyl)piperidin-1-yl]propan-2-ol | |
| 411 | 1-(3-{3-[(2-Piperidin-1-ylethyl)sulfanyl]-4-(trifluoromethyl)phenyl}-4,5,6,7- | 649.34 |
| | tetrahydro-1H-pyrazolo[4,3-c]pyridin-1-yl)-3-[4-(pyrrolidin-1- | |
| | ylcarbonyl)piperidin-1-yl]propan-2-ol | |
| 412 | (2S)-1-{3-[3-{[2-(3-Fluoropiperidin-1-yl)ethyl]sulfanyl}-4- | 650.20 |
| | (trifluoromethyl)phenyl]-5-(methylsulfonyl)-4,5,6,7-tetrahydro-1H- | |
| | pyrazolo[4,3-c]pyridin-1-yl}-3-morpholin-4-ylpropan-2-ol | |
| 413 | (3S)-1-(3-{3-[3-{[2-(4-Fluoropiperidin-1-yl)ethyl]sulfanyl}-4- | 650.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)pyrrolidin-3-ol | |
| 414 | (3R)-1-(3-{3-[3-{[2-(4-Fluoropiperidin-1-yl)ethyl]sulfanyl}-4- | 650.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)pyrrolidin-3-ol | |
| 415 | N-Methyl-2-({5-[5-(methylsulfonyl)-1-(3-piperidin-1-ylpropyl)-4,5,6,7- | 650.30 |
| | tetrahydro-1H-pyrazolo[4,3-c]pyridin-3-yl]-2- | |
| | (trifluoromethyl)phenyl}sulfanyl)-N-(phenylmethyl)ethanamine | |
| 416 | 1-{3-[3-{[2-(3,3-Difluoropyrrolidin-1-yl)ethyl]sulfanyl}-4- | 650.30 |
| | (trifluoromethyl)phenyl]-4,5,6,7-tetrahydro-1H-pyrazolo[4,3-c]pyridin-1- | |
| | yl}-3-(4-phenylpiperidin-1-yl)propan-2-ol | |
| 417 | 1-{3-[3-{[2-(3,3-Difluoropyrrolidin-1-yl)ethyl]sulfanyl}-4- | 651.30 |
| | (trifluoromethyl)phenyl]-4,5,6,7-tetrahydro-1H-pyrazolo[4,3-c]pyridin-1- | |
| | yl}-3-(4-phenylpiperazin-1-yl)propan-2-ol | |
| 418 | 1-{3-[3-{[2-(3,3-Difluoropyrrolidin-1-yl)ethyl]sulfanyl}-4- | 651.30 |
| | (trifluoromethyl)phenyl]-4,5,6,7-tetrahydro-1H-pyrazolo[4,3-c]pyridin-1- | |
| | yl}-3-(4-pyridin-2-ylpiperidin-1-yl)propan-2-ol | |
| 419 | 1-[1-(2-Hydroxy-3-{3-[3-{[2-(3-hydroxypiperidin-1-yl)ethyl]sulfanyl}-4- | 651.40 |
| | (trifluoromethyl)phenyl]-4,5,6,7-tetrahydro-1H-pyrazolo[4,3-c]pyridin-1- | |
| | yl}propyl)piperidin-4-yl]pyrrolidin-2-one | |
| 420 | (4R)-4-Hydroxy-1-{1-[(2S)-2-hydroxy-3-(5-methyl-3-{3-[(2-pyrrolidin-1- | 651.70 |
| | ylethyl)sulfanyl]-4-(trifluoromethyl)phenyl}-4,5,6,7-tetrahydro-1H- | |
| | pyrazolo[4,3-c]pyridin-1-yl)propyl]piperidin-4-yl}pyrrolidin-2-one | |
| 421 | 1-{3-[3-{[2-(3,3-Difluoropyrrolidin-1-yl)ethyl]sulfanyl}-4- | 652.20 |
| | (trifluoromethyl)phenyl]-5-(methylsulfonyl)-4,5,6,7-tetrahydro-1H- | |
| | pyrazolo[4,3-c]pyridin-1-yl}-3-piperidin-1-ylpropan-2-ol | |
| 422 | 1-(5-Acetyl-3-{3-[(2-oxo-2-pyrrolidin-1-ylethyl)sulfanyl]-4- | 652.25 |
| | (trifluoromethyl)phenyl}-4,5,6,7-tetrahydro-1H-pyrazolo[4,3-c]pyridin-1- | |
| | yl)-3-[4-(1-hydroxy-1-methylethyl)piperidin-1-yl]propan-2-ol | |
| 423 | 1-(5-Acetyl-3-{3-[(2-oxo-2-pyrrolidin-1-ylethyl)sulfanyl]-4- | 652.27 |
| | (trifluoromethyl)phenyl}-4,5,6,7-tetrahydro-1H-pyrazolo[4,3-c]pyridin-1- | |
| | yl)-3-(1,4-dioxa-8-azaspiro[4.5]dec-8-yl)propan-2-ol | |
| 424 | (2S)-1-{5-(Cyclobutylcarbonyl)-3-[3-{[2-(4-fluoropiperidin-1- | 652.32 |
| | yl)ethyl]sulfanyl}-4-(trifluoromethyl)phenyl]-4,5,6,7-tetrahydro-1H- | |
| | pyrazolo[4,3-c]pyridin-1-yl}-3-piperidin-1-ylpropan-2-ol | |
| 425 | 4-{1-[2-Hydroxy-3-(3-{3-[(2-oxo-2-pyrrolidin-1-ylethyl)sulfanyl]-4- | 653.28 |
| | (trifluoromethyl)phenyl}-4,5,6,7-tetrahydro-1H-pyrazolo[4,3-c]pyridin-1- | |
| | yl)propyl]piperidin-4-yl}benzonitrile | |
| 426 | 3-{1-[2-Hydroxy-3-(3-{3-[(2-piperidin-1-ylethyl)sulfanyl]-4- | 653.30 |
| | (trifluoromethyl)phenyl}-4,5,6,7-tetrahydro-1H-pyrazolo[4,3-c]pyridin-1- | |
| | yl)propyl]piperidin-4-yl}benzonitrile | |
| 427 | 4-{1-[2-Hydroxy-3-(3-{3-[(2-piperidin-1-ylethyl)sulfanyl]-4- | 653.32 |
| | (trifluoromethyl)phenyl}-4,5,6,7-tetrahydro-1H-pyrazolo[4,3-c]pyridin-1- | |
| | yl)propyl]piperidin-4-yl}benzonitrile | |
| 428 | 1-{5-[(1-Aminocyclopropyl)carbonyl]-3-[3-{[2-(4-fluoropiperidin-1- | 653.32 |
| | yl)ethyl]sulfanyl}-4-(trifluoromethyl)phenyl]-4,5,6,7-tetrahydro-1H- | |
| | pyrazolo[4,3-c]pyridin-1-yl}-3-piperidin-1-ylpropan-2-ol | |
| 429 | 1-{1-[(2S)-3-{3-[3-{[2-(4-Fluoropiperidin-1-yl)ethyl]sulfanyl}-4- | 654.10 |
| | (trifluoromethyl)phenyl]-4,5,6,7-tetrahydro-1H-pyrazolo[4,3-c]pyridin-1- | |
| | yl}-2-hydroxypropyl]piperidin-4-yl}pyrrolidin-2-one | |
| 430 | N-(2-{[5-{1-[2-Hydroxy-3-(4-methylpiperidin-1-yl)propyl]-5- | 654.20 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)methanesulfonamide | |
| 431 | 2-{4-[2-Hydroxy-3-(3-{3-[(2-piperidin-1-ylethyl)sulfanyl]-4- | 654.31 |
| | (trifluoromethyl)phenyl}-4,5,6,7-tetrahydro-1H-pyrazolo[4,3-c]pyridin-1- | |
| | yl)propyl]piperazin-1-yl}benzonitrile | |
| 432 | 2-{3-[3-{[2-(4-Fluoropiperidin-1-yl)ethyl]sulfanyl}-4- | 655.30 |
| | (trifluoromethyl)phenyl]-1-[(2S)-2-hydroxy-3-piperidin-1-ylpropyl]-1,4,6,7- | |
| | tetrahydro-5H-pyrazolo[4,3-c]pyridin-5-yl}-N-methyl-2-oxoacetamide | |
| 433 | 1-{5-(N,N-Dimethylglycyl)-3-[3-{[2-(4-fluoropiperidin-1-yl)ethyl]sulfanyl}- | 655.30 |
| | 4-(trifluoromethyl)phenyl]-4,5,6,7-tetrahydro-1H-pyrazolo[4,3-c]pyridin-1- | |
| | yl}-3-piperidin-1-ylpropan-2-ol | |
| 434 | 3-[3-{[2-(4-Fluoropiperidin-1-yl)ethyl]sulfanyl}-4-(trifluoromethyl)phenyl]- | 655.33 |
| | 1-[(2S)-2-hydroxy-3-piperidin-1-ylpropyl]-N-(1-methylethyl)-1,4,6,7- | |
| | tetrahydro-5H-pyrazolo[4,3-c]pyridine-5-carboxamide | |
| 435 | Methyl {3-[3-{[2-(4-fluoropiperidin-1-yl)ethyl]sulfanyl}-4- | 656.20 |
| | (trifluoromethyl)phenyl]-1-(2-hydroxy-3-piperidin-1-ylpropyl)-1,4,6,7- | |
| | tetrahydro-5H-pyrazolo[4,3-c]pyridin-5-yl}(oxo)acetate | |
| 436 | Methyl {3-[3-{[2-(4-fluoropiperidin-1-yl)ethyl]sulfanyl}-4- | 656.30 |
| | (trifluoromethyl)phenyl]-1-[(2S)-2-hydroxy-3-piperidin-1-ylpropyl]-1,4,6,7- | |
| | tetrahydro-5H-pyrazolo[4,3-c]pyridin-5-yl}(oxo)acetate | |
| 437 | 1-{3-[3-{[2-(4-Fluoropiperidin-1-yl)ethyl]sulfanyl}-4- | 656.32 |
| | (trifluoromethyl)phenyl]-1-(2-hydroxy-3-piperidin-1-ylpropyl)-1,4,6,7- | |
| | tetrahydro-5H-pyrazolo[4,3-c]pyridin-5-yl}-2-methyl-1-oxopropan-2-ol | |
| 438 | 1-(4-Cyclopropylpiperazin-1-yl)-3-[5-(methylsulfonyl)-3-{3-[(2-pyrrolidin- | 657.28 |
| | 1-ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 439 | 1-[1-(3-{3-[3-{[2-(3,3-Difluoropyrrolidin-1-yl)ethyl]sulfanyl}-4- | 657.30 |
| | (trifluoromethyl)phenyl]-4,5,6,7-tetrahydro-1H-pyrazolo[4,3-c]pyridin-1- | |
| | yl}-2-hydroxypropyl)piperidin-4-yl]pyrrolidin-2-one | |
| 440 | N-(2-{[5-{5-(Methylsulfonyl)-1-[3-(4-pyrrolidin-1-ylpiperidin-1-yl)propyl]- | 657.30 |
| | 4,5,6,7-tetrahydro-1H-pyrazolo[4,3-c]pyridin-3-yl}-2- | |
| | (trifluoromethyl)phenyl]sulfanyl}ethyl)propan-1-amine | |
| 441 | (2S)-1-{3-[3-{[2-(4-Fluoropiperidin-1-yl)ethyl]sulfanyl}-4- | 658.28 |
| | (trifluoromethyl)phenyl]-5-[(methylsulfanyl)acetyl]-4,5,6,7-tetrahydro-1H- | |
| | pyrazolo[4,3-c]pyridin-1-yl}-3-piperidin-1-ylpropan-2-ol | |
| 442 | 1-{3-[3-{[2-(4-Fluoropiperidin-1-yl)ethyl]sulfanyl}-4- | 658.28 |
| | (trifluoromethyl)phenyl]-5-[(methylsulfanyl)acetyl]-4,5,6,7-tetrahydro-1H- | |
| | pyrazolo[4,3-c]pyridin-1-yl}-3-piperidin-1-ylpropan-2-ol | |
| 443 | 1-{4-[2-(Methyloxy)phenyl]piperidin-1-yl}-3-(3-{3-[(2-oxo-2-pyrrolidin-1- | 658.30 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 444 | (2S)-1-(4-Methylpiperidin-1-yl)-3-{3-[3-{[2-(4-methylpiperidin-1- | 658.30 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 445 | (2S)-1-Azepan-1-yl-3-{3-[3-{[2-(4-methylpiperidin-1-yl)ethyl]sulfanyl}-4- | 658.30 |
| | (trifluoromethyl)phenyl]-5-(methylsulfonyl)-4,5,6,7-tetrahydro-1H- | |
| | pyrazolo[4,3-c]pyridin-1-yl}propan-2-ol | |
| 446 | 1-{4-[4-(Methyloxy)phenyl]piperidin-1-yl}-3-(3-{3-[(2-piperidin-1- | 658.33 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 447 | 1-{4-[2-(Methyloxy)phenyl]piperidin-1-yl}-3-(3-{3-[(2-piperidin-1- | 658.40 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 448 | 1-{3-[3-{[2-(4-Acetylpiperazin-1-yl)ethyl]sulfanyl}-4- | 659.20 |
| | (trifluoromethyl)phenyl]-5-(methylsulfonyl)-4,5,6,7-tetrahydro-1H- | |
| | pyrazolo[4,3-c]pyridin-1-yl}-3-pyrrolidin-1-ylpropan-2-ol | |
| 449 | 1-(4-Acetylpiperazin-1-yl)-3-[5-(methylsulfonyl)-3-{3-[(2-pyrrolidin-1- | 659.26 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 450 | N-(1-{2-Hydroxy-3-[5-(methylsulfonyl)-3-{3-[(2-pyrrolidin-1- | 659.26 |
| | ylethyl)sulfanyl]-4-(trifluoromethyl)phenyl}-4,5,6,7-tetrahydro-1H- | |
| | pyrazolo[4,3-c]pyridin-1-yl]propyl}pyrrolidin-3-yl)acetamide | |
| 451 | 1-{4-[3-(Methyloxy)phenyl]piperazin-1-yl}-3-(3-{3-[(2-piperidin-1- | 659.33 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 452 | 1-{4-[4-(Methyloxy)phenyl]piperazin-1-yl}-3-(3-{3-[(2-piperidin-1- | 659.33 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 453 | 1-{4-[2-(Methyloxy)phenyl]piperazin-1-yl}-3-(3-{3-[(2-piperidin-1- | 659.33 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 454 | (2S)-1-{3-[3-({2-[(3S)-3-Fluoropyrrolidin-1-yl]ethyl}sulfanyl)-4- | 660.20 |
| | (trifluoromethyl)phenyl]-5-(methylsulfonyl)-4,5,6,7-tetrahydro-1H- | |
| | pyrazolo[4,3-c]pyridin-1-yl}-3-(hexahydrocyclopenta[c]pyrrol-2(1H)- | |
| | yl)propan-2-ol | |
| 455 | 1-{2-Hydroxy-3-[5-(methylsulfonyl)-3-{3-[(2-piperidin-1-ylethyl)sulfanyl]- | 660.20 |
| | 4-(trifluoromethyl)phenyl}-4,5,6,7-tetrahydro-1H-pyrazolo[4,3-c]pyridin-1- | |
| | yl]propyl}praline | |
| 456 | (2S)-1-[(3S)-3-Methylmorpholin-4-yl]-3-{3-[3-{[2-(4-methylpiperidin-1- | 660.28 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 457 | 1-{3-[3-({2-[3-(Hydroxymethyl)piperidin-1-yl]ethyl}sulfanyl)-4- | 660.28 |
| | (trifluoromethyl)phenyl]-5-(methylsulfonyl)-4,5,6,7-tetrahydro-1H- | |
| | pyrazolo[4,3-c]pyridin-1-yl}-3-piperidin-1-ylpropan-2-ol | |
| 458 | 1-(3,3-Dimethylpiperidin-1-yl)-3-[5-(methylsulfonyl)-3-{3-[(2-morpholin-4- | 660.30 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 459 | 4-{2-Hydroxy-3-[5-(methylsulfonyl)-3-{3-[(2-morpholin-4-ylethyl)sulfanyl]- | 661.20 |
| | 4-(trifluoromethyl)phenyl}-4,5,6,7-tetrahydro-1H-pyrazolo[4,3-c]pyridin-1- | |
| | yl]propyl}-3-methylpiperazin-2-one | |
| 460 | N-{1-[2-Hydroxy-3-(3-{3-[(2-morpholin-4-yl-2-oxoethyl)sulfanyl]-4- | 661.20 |
| | (trifluoromethyl)phenyl}-4,5,6,7-tetrahydro-1H-pyrazolo[4,3-c]pyridin-1- | |
| | yl)propyl]piperidin-4-yl}methanesulfonamide | |
| 461 | 1-[2-Hydroxy-3-(3-{3-[(2-morpholin-4-yl-2-oxoethyl)sulfanyl]-4- | 661.27 |
| | (trifluoromethyl)phenyl}-4,5,6,7-tetrahydro-1H-pyrazolo[4,3-c]pyridin-1- | |
| | yl)propyl]-4-pyridin-2-ylpiperidin-4-ol | |
| 462 | 1-(2-{[5-{1-[2-Hydroxy-3-(4-hydroxy-4-pyridin-2-ylpiperidin-1-yl)propyl]- | 661.40 |
| | 4,5,6,7-tetrahydro-1H-pyrazolo[4,3-c]pyridin-3-yl}-2- | |
| | (trifluoromethyl)phenyl]sulfanyl}ethyl)piperidin-3-ol | |
| 463 | N-[1-(2-Hydroxy-3-{3-[3-{[2-(3-hydroxypiperidin-1-yl)ethyl]sulfanyl}-4- | 661.40 |
| | (trifluoromethyl)phenyl]-4,5,6,7-tetrahydro-1H-pyrazolo[4,3-c]pyridin-1- | |
| | yl}propyl)piperidin-4-yl]methanesulfonamide | |
| 464 | (2S)-1-(4,4-Dimethylpiperidin-1-yl)-3-{3-[3-({2-[(3S)-3-fluoropyrrolidin-1- | 662.20 |
| | yl]ethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 465 | 1-[(3S)-3-(Hydroxymethyl)pyrrolidin-1-yl]-3-[5-(methylsulfonyl)-3-{3-[(2- | 662.22 |
| | morpholin-4-yl-2-oxoethyl)sulfanyl]-4-(trifluoromethyl)phenyl}-4,5,6,7- | |
| | tetrahydro-1H-pyrazolo[4,3-c]pyridin-1-yl]propan-2-ol | |
| 466 | 1-[(3S)-3-Methylmorpholin-4-yl]-3-[5-(methylsulfonyl)-3-{3-[(2-morpholin- | 662.22 |
| | 4-yl-2-oxoethyl)sulfanyl]-4-(trifluoromethyl)phenyl}-4,5,6,7-tetrahydro- | |
| | 1H-pyrazolo[4,3-c]pyridin-1-yl]propan-2-ol | |
| 467 | (2S)-1-{3-[3-{[2-(4-Methylpiperidin-1-yl)ethyl]sulfanyl}-4- | 662.24 |
| | (trifluoromethyl)phenyl]-5-(methylsulfonyl)-4,5,6,7-tetrahydro-1H- | |
| | pyrazolo[4,3-c]pyridin-1-yl}-3-thiomorpholin-4-ylpropan-2-ol | |
| 468 | 1-[4-(3-Chlorophenyl)piperidin-1-yl]-3-(3-{3-[(2-piperidin-1- | 662.28 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 469 | 1-[4-(Methyloxy)piperidin-1-yl]-3-[5-(methylsulfonyl)-3-{3-[(2-morpholin- | 662.30 |
| | 4-ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 470 | (2S)-1-Azepan-1-yl-3-{3-[3-{[2-(3-fluoropiperidin-1-yl)ethyl]sulfanyl}-4- | 662.30 |
| | (trifluoromethyl)phenyl]-5-(methylsulfonyl)-4,5,6,7-tetrahydro-1H- | |
| | pyrazolo[4,3-c]pyridin-1-yl}propan-2-ol | |
| 471 | (2S)-1-{3-[3-{[2-(3-Fluoropiperidin-1-yl)ethyl]sulfanyl}-4- | 662.30 |
| | (trifluoromethyl)phenyl]-5-(methylsulfonyl)-4,5,6,7-tetrahydro-1H- | |
| | pyrazolo[4,3-c]pyridin-1-yl}-3-(4-methylpiperidin-1-yl)propan-2-ol | |
| 472 | (2S)-1-Azepan-1-yl-3-{3-[3-{[2-(4-fluoropiperidin-1-yl)ethyl]sulfanyl}-4- | 662.30 |
| | (trifluoromethyl)phenyl]-5-(methylsulfonyl)-4,5,6,7-tetrahydro-1H- | |
| | pyrazolo[4,3-c]pyridin-1-yl}propan-2-ol | |
| 473 | 1-{3-[3-{[2-(4-Fluoropiperidin-1-yl)ethyl]sulfanyl}-4- | 662.30 |
| | (trifluoromethyl)phenyl]-5-(methylsulfonyl)-4,5,6,7-tetrahydro-1H- | |
| | pyrazolo[4,3-c]pyridin-1-yl}-3-(4-methylpiperidin-1-yl)propan-2-ol | |
| 474 | (2S)-1-{3-[3-{[2-(4-Fluoropiperidin-1-yl)ethyl]sulfanyl}-4- | 662.30 |
| | (trifluoromethyl)phenyl]-5-(methylsulfonyl)-4,5,6,7-tetrahydro-1H- | |
| | pyrazolo[4,3-c]pyridin-1-yl}-3-(4-methylpiperidin-1-yl)propan-2-ol | |
| 475 | 1-(2-{[5-(1-{2-Hydroxy-3-[4-(pyrimidin-2-yloxy)piperidin-1-yl]propyl}- | 662.40 |
| | 4,5,6,7-tetrahydro-1H-pyrazolo[4,3-c]pyridin-3-yl)-2- | |
| | (trifluoromethyl)phenyl]sulfanyl}ethyl)piperidin-3-ol | |
| 476 | 1-(4-Aminopiperidin-1-yl)-3-{3-[3-{[2-(4-fluoropiperidin-1- | 663.20 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 477 | N-{1-[(2S)-3-{3-[3-{[2-(4-Fluoropiperidin-1-yl)ethyl]sulfanyl}-4- | 663.27 |
| | (trifluoromethyl)phenyl]-4,5,6,7-tetrahydro-1H-pyrazolo[4,3-c]pyridin-1- | |
| | yl}-2-hydroxypropyl]piperidin-4-yl}methanesulfonamide | |
| 478 | 2-{4-[(2S)-3-{3-[3-{[2-(4-Fluoropiperidin-1-yl)ethyl]sulfanyl}-4- | 663.30 |
| | (trifluoromethyl)phenyl]-4,5,6,7-tetrahydro-1H-pyrazolo[4,3-c]pyridin-1- | |
| | yl}-2-hydroxypropyl]piperazin-1-yl}phenol | |
| 479 | 3-{4-[(2S)-3-{3-[3-{[2-(4-Fluoropiperidin-1-yl)ethyl]sulfanyl}-4- | 664.00 |
| | (trifluoromethyl)phenyl]-4,5,6,7-tetrahydro-1H-pyrazolo[4,3-c]pyridin-1- | |
| | yl}-2-hydroxypropyl]piperazin-1-yl}phenol | |
| 480 | 1-[(2S)-3-{3-[3-{[2-(4-Fluoropiperidin-1-yl)ethyl]sulfanyl}-4- | 664.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-4-ol | |
| 481 | (3S)-1-[(2S)-3-{3-[3-{[2-(4-Fluoropiperidin-1-yl)ethyl]sulfanyl}-4- | 664.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-3-ol | |
| 482 | 1-[2-(Hydroxymethyl)morpholin-4-yl]-3-[5-(methylsulfonyl)-3-{3-[(2- | 664.20 |
| | morpholin-4-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 483 | (3R)-1-(3-{3-[3-{[2-(4-Fluoropiperidin-1-yl)ethyl]sulfanyl}-4- | 664.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidin-3-ol | |
| 484 | 1-[(2S)-3-{3-[3-({2-[(3S)-3-Fluoropyrrolidin-1-yl]ethyl}sulfanyl)-4- | 664.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]-4-methylpiperidin-4-ol | |
| 485 | (2S)-1-{3-[3-{[2-(4,4-Difluoropiperidin-1-yl)ethyl]sulfanyl}-4- | 664.20 |
| | (trifluoromethyl)phenyl]-5-(methylsulfonyl)-4,5,6,7-tetrahydro-1H- | |
| | pyrazolo[4,3-c]pyridin-1-yl}-3-(3,6-dihydropyridin-1(2H)-yl)propan-2-ol | |
| 486 | 1-(3-{3-[3-{[2-(4-Fluoropiperidin-1-yl)ethyl]sulfanyl}-4- | 664.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidin-3-ol | |
| 487 | (3R)-1-[(2S)-3-{3-[3-{[2-(4-Fluoropiperidin-1-yl)ethyl]sulfanyl}-4- | 664.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-3-ol | |
| 488 | 1-(3-{3-[3-{[2-(4-Fluoropiperidin-1-yl)ethyl]sulfanyl}-4- | 664.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidin-4-ol | |
| 489 | N-Methyl-2-({5-[5-(methylsulfonyl)-1-(3-piperidin-1-ylpropyl)-4,5,6,7- | 664.30 |
| | tetrahydro-1H-pyrazolo[4,3-c]pyridin-3-yl]-2- | |
| | (trifluoromethyl)phenyl}sulfanyl)-N-(2-phenylethyl)ethanamine | |
| 490 | 1-{3-[3-{[2-(4-Fluoropiperidin-1-yl)ethyl]sulfanyl}-4- | 664.30 |
| | (trifluoromethyl)phenyl]-4,5,6,7-tetrahydro-1H-pyrazolo[4,3-c]pyridin-1- | |
| | yl}-3-[4-(pyrimidin-2-yloxy)piperidin-1-yl]propan-2-ol | |
| 491 | 1-{3-[3-{[2-(4-Fluoropiperidin-1-yl)ethyl]sulfanyl}-4- | 664.30 |
| | (trifluoromethyl)phenyl]-5-(methylsulfonyl)-4,5,6,7-tetrahydro-1H- | |
| | pyrazolo[4,3-c]pyridin-1-yl}-3-[(2S)-2-(hydroxymethyl)pyrrolidin-1- | |
| | yl]propan-2-ol | |
| 492 | (2S)-1-{3-[3-{[2-(3,3-Difluoropiperidin-1-yl)ethyl]sulfanyl}-4- | 665.20 |
| | (trifluoromethyl)phenyl]-5-(methylsulfonyl)-4,5,6,7-tetrahydro-1H- | |
| | pyrazolo[4,3-c]pyridin-1-yl}-3-(5,6-dihydropyrimidin-1(4H)-yl)propan-2-ol | |
| 493 | (2S)-1-{3-[3-{[2-(4,4-Difluoropiperidin-1-yl)ethyl]sulfanyl}-4- | 665.20 |
| | (trifluoromethyl)phenyl]-5-(methylsulfonyl)-4,5,6,7-tetrahydro-1H- | |
| | pyrazolo[4,3-c]pyridin-1-yl}-3-(5,6-dihydropyrimidin-1(4H)-yl)propan-2-ol | |
| 494 | (2S)-1-{3-[3-{[2-(4-Fluoropiperidin-1-yl)ethyl]sulfanyl}-4- | 665.30 |
| | (trifluoromethyl)phenyl]-5-(1H-1,2,4-triazol-3-ylcarbonyl)-4,5,6,7- | |
| | tetrahydro-1H-pyrazolo[4,3-c]pyridin-1-yl}-3-piperidin-1-ylpropan-2-ol | |
| 495 | 1-(3-{3-[(2-Morpholin-4-yl-2-oxoethyl)sulfanyl]-4-(trifluoromethyl)phenyl}- | 665.30 |
| | 4,5,6,7-tetrahydro-1H-pyrazolo[4,3-c]pyridin-1-yl)-3-[4-(pyrrolidin-1- | |
| | ylcarbonyl)piperidin-1-yl]propan-2-ol | |
| 496 | 1-[4-(Morpholin-4-ylcarbonyl)piperidin-1-yl]-3-(3-{3-[(2-piperidin-1- | 665.34 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 497 | 1-(2-{[5-(1-{2-Hydroxy-3-[4-(pyrrolidin-1-ylcarbonyl)piperidin-1-yl]propyl}- | 665.40 |
| | 4,5,6,7-tetrahydro-1H-pyrazolo[4,3-c]pyridin-3-yl)-2- | |
| | (trifluoromethyl)phenyl]sulfanyl}ethyl)piperidin-3-ol | |
| 498 | (2S)-1-[4-(2-Fluorophenyl)piperazin-1-yl]-3-{3-[3-{[2-(4-fluoropiperidin-1- | 666.10 |
| | yl)ethyl]sulfanyl}-4-(trifluoromethyl)phenyl]-4,5,6,7-tetrahydro-1H- | |
| | pyrazolo[4,3-c]pyridin-1-yl}propan-2-ol | |
| 499 | (2S)-1-[4-(4-Fluorophenyl)piperazin-1-yl]-3-{3-[3-{[2-(4-fluoropiperidin-1- | 666.10 |
| | yl)ethyl]sulfanyl}-4-(trifluoromethyl)phenyl]-4,5,6,7-tetrahydro-1H- | |
| | pyrazolo[4,3-c]pyridin-1-yl}propan-2-ol | |
| 500 | (2S)-1-(3-Fluoropiperidin-1-yl)-3-{3-[3-{[2-(4-fluoropiperidin-1- | 666.20 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 501 | 1-{3-[3-{[2-(3,3-Difluoropyrrolidin-1-yl)ethyl]sulfanyl}-4- | 666.20 |
| | (trifluoromethyl)phenyl]-5-(methylsulfonyl)-4,5,6,7-tetrahydro-1H- | |
| | pyrazolo[4,3-c]pyridin-1-yl}-3-(4-methylpiperidin-1-yl)propan-2-ol | |
| 502 | (2S)-1-{3-[3-{[2-(3-Fluoropiperidin-1-yl)ethyl]sulfanyl}-4- | 666.20 |
| | (trifluoromethyl)phenyl]-5-(methylsulfonyl)-4,5,6,7-tetrahydro-1H- | |
| | pyrazolo[4,3-c]pyridin-1-yl}-3-thiomorpholin-4-ylpropan-2-ol | |
| 503 | (2S)-1-(4-Fluoropiperidin-1-yl)-3-{3-[3-{[2-(4-fluoropiperidin-1- | 666.20 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 504 | (2S)-1-(4-Fluoropiperidin-1-yl)-3-{3-[3-{[2-(3-fluoropiperidin-1- | 666.20 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 505 | N-[1-(3-{3-[3-{[2-(3,3-Difluoropyrrolidin-1-yl)ethyl]sulfanyl}-4- | 667.20 |
| | (trifluoromethyl)phenyl]-4,5,6,7-tetrahydro-1H-pyrazolo[4,3-c]pyridin-1- | |
| | yl}-2-hydroxypropyl)piperidin-4-yl]methanesulfonamide | |
| 506 | 4-[4-(3-{3-[3-{[2-(3,3-Difluoropyrrolidin-1-yl)ethyl]sulfanyl}-4- | 667.30 |
| | (trifluoromethyl)phenyl]-4,5,6,7-tetrahydro-1H-pyrazolo[4,3-c]pyridin-1- | |
| | yl}-2-hydroxypropyl)piperazin-1-yl]phenol | |
| 507 | 1-(3-{3-[3-{[2-(3,3-Difluoropyrrolidin-1-yl)ethyl]sulfanyl}-4- | 667.30 |
| | (trifluoromethyl)phenyl]-4,5,6,7-tetrahydro-1H-pyrazolo[4,3-c]pyridin-1- | |
| | yl}-2-hydroxypropyl)-4-pyridin-2-ylpiperidin-4-ol | |
| 508 | 1-{3-[3-{[2-(4-Fluoropiperidin-1-yl)ethyl]sulfanyl}-4- | 667.30 |
| | (trifluoromethyl)phenyl]-4,5,6,7-tetrahydro-1H-pyrazolo[4,3-c]pyridin-1- | |
| | yl}-3-[4-(pyrrolidin-1-ylcarbonyl)piperidin-1-yl]propan-2-ol | |
| 509 | 3-[4-(3-{3-[3-{[2-(3,3-Difluoropyrrolidin-1-yl)ethyl]sulfanyl}-4- | 667.30 |
| | (trifluoromethyl)phenyl]-4,5,6,7-tetrahydro-1H-pyrazolo[4,3-c]pyridin-1- | |
| | yl}-2-hydroxypropyl)piperazin-1-yl]phenol | |
| 510 | 2-[4-(3-{3-[3-{[2-(3,3-Difluoropyrrolidin-1-yl)ethyl]sulfanyl}-4- | 667.30 |
| | (trifluoromethyl)phenyl]-4,5,6,7-tetrahydro-1H-pyrazolo[4,3-c]pyridin-1- | |
| | yl}-2-hydroxypropyl)piperazin-1-yl]pyridine-3-carbonitrile | |
| 511 | (2S)-1-{3-[3-{[2-(4-Fluoropiperidin-1-yl)ethyl]sulfanyl}-4- | 667.33 |
| | (trifluoromethyl)phenyl]-5-(pyrrolidin-1-ylcarbonyl)-4,5,6,7-tetrahydro-1H- | |
| | pyrazolo[4,3-c]pyridin-1-yl}-3-piperidin-1-ylpropan-2-ol | |
| 512 | (2S)-1-{3-[3-{[2-(4-Fluoropiperidin-1-yl)ethyl]sulfanyl}-4- | 668.10 |
| | (trifluoromethyl)phenyl]-4,5,6,7-tetrahydro-1H-pyrazolo[4,3-c]pyridin-1- | |
| | yl}-3-[4-(pyrrolidin-1-ylcarbonyl)piperidin-1-yl]propan-2-ol | |
| 513 | (2S)-1-{3-[3-{[2-(3,3-Difluoropiperidin-1-yl)ethyl]sulfanyl}-4- | 668.20 |
| | (trifluoromethyl)phenyl]-5-(methylsulfonyl)-4,5,6,7-tetrahydro-1H- | |
| | pyrazolo[4,3-c]pyridin-1-yl}-3-morpholin-4-ylpropan-2-ol | |
| 514 | 1-(3-{3-[3-{[2-(3,3-Difluoropyrrolidin-1-yl)ethyl]sulfanyl}-4- | 668.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidin-4-ol | |
| 515 | (2S)-1-{3-[3-{[2-(4,4-Difluoropiperidin-1-yl)ethyl]sulfanyl}-4- | 668.20 |
| | (trifluoromethyl)phenyl]-5-(methylsulfonyl)-4,5,6,7-tetrahydro-1H- | |
| | pyrazolo[4,3-c]pyridin-1-yl}-3-morpholin-4-ylpropan-2-ol | |
| 516 | N-(2-{[5-{1-[3-(4,4-Dimethylpiperidin-1-yl)-2-hydroxypropyl]-5- | 668.20 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)methanesulfonamide | |
| 517 | 1-(4,4-Difluoropiperidin-1-yl)-3-[5-(methylsulfonyl)-3-{3-[(2-morpholin-4- | 668.20 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 518 | 1-(3,3-Difluoropiperidin-1-yl)-3-[5-(methylsulfonyl)-3-{3-[(2-morpholin-4- | 668.20 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 519 | 1-{3-[3-{[2-(3,3-Difluoropyrrolidin-1-yl)ethyl]sulfanyl}-4- | 668.30 |
| | (trifluoromethyl)phenyl]-4,5,6,7-tetrahydro-1H-pyrazolo[4,3-c]pyridin-1- | |
| | yl}-3-[4-(pyrimidin-2-yloxy)piperidin-1-yl]propan-2-ol | |
| 520 | 1-{3-[3-{[2-(4-Fluoropiperidin-1-yl)ethyl]sulfanyl}-4- | 668.32 |
| | (trifluoromethyl)phenyl]-5-[(2R)-tetrahydrofuran-2-ylcarbonyl]-4,5,6,7- | |
| | tetrahydro-1H-pyrazolo[4,3-c]pyridin-1-yl}-3-piperidin-1-ylpropan-2-ol | |
| 521 | 1-{3-[3-{[2-(3,3-Difluoropyrrolidin-1-yl)ethyl]sulfanyl}-4- | 669.30 |
| | (trifluoromethyl)phenyl]-4,5,6,7-tetrahydro-1H-pyrazolo[4,3-c]pyridin-1- | |
| | yl}-3-[4-(4-fluorophenyl)piperazin-1-yl]propan-2-ol | |
| 522 | N-(1,1-Dimethylethyl)-3-[3-{[2-(4-fluoropiperidin-1-yl)ethyl]sulfanyl}-4- | 669.30 |
| | (trifluoromethyl)phenyl]-1-[(2S)-2-hydroxy-3-piperidin-1-ylpropyl]-1,4,6,7- | |
| | tetrahydro-5H-pyrazolo[4,3-c]pyridine-5-carboxamide | |
| 523 | 3-[3-{[2-(4-Fluoropiperidin-1-yl)ethyl]sulfanyl}-4-(trifluoromethyl)phenyl]- | 669.30 |
| | 1-[(2S)-2-hydroxy-3-piperidin-1-ylpropyl]-N-(1-methylpropyl)-1,4,6,7- | |
| | tetrahydro-5H-pyrazolo[4,3-c]pyridine-5-carboxamide | |
| 524 | 1-[1-(3-{3-[3-{[2-(Cyclopropylamino)ethyl]sulfanyl}-4- | 669.30 |
| | (trifluoromethyl)phenyl]-5-(methylsulfonyl)-4,5,6,7-tetrahydro-1H- | |
| | pyrazolo[4,3-c]pyridin-1-yl}propyl)piperidin-4-yl]pyrrolidin-2-one | |
| 525 | N,N-Diethyl-3-[3-{[2-(4-fluoropiperidin-1-yl)ethyl]sulfanyl}-4- | 669.30 |
| | (trifluoromethyl)phenyl]-1-[(2S)-2-hydroxy-3-piperidin-1-ylpropyl]-1,4,6,7- | |
| | tetrahydro-5H-pyrazolo[4,3-c]pyridine-5-carboxamide | |
| 526 | 2-{4-[2-Hydroxy-3-(3-{3-[(2-morpholin-4-yl-2-oxoethyl)sulfanyl]-4- | 670.20 |
| | (trifluoromethyl)phenyl}-4,5,6,7-tetrahydro-1H-pyrazolo[4,3-c]pyridin-1- | |
| | yl)propyl]piperazin-1-yl}benzonitrile | |
| 527 | 1-[5-(Methylsulfonyl)-3-{3-[(2-morpholin-4-ylethyl)sulfanyl]-4- | 670.20 |
| | (trifluoromethyl)phenyl}-4,5,6,7-tetrahydro-1H-pyrazolo[4,3-c]pyridin-1- | |
| | yl]-3-(2,4,6,7-tetrahydro-5H-pyrazolo[4,3-c]pyridin-5-yl)propan-2-ol | |
| 528 | 1-[5-(Methylsulfonyl)-3-{3-[(2-morpholin-4-ylethyl)sulfanyl]-4- | 670.20 |
| | (trifluoromethyl)phenyl}-4,5,6,7-tetrahydro-1H-pyrazolo[4,3-c]pyridin-1- | |
| | yl]-3-(1,4,6,7-tetrahydro-5H-imidazo[4,5-c]pyridin-5-yl)propan-2-ol | |
| 529 | N-(1-{3-[5-(Methylsulfonyl)-3-{3-[(2-piperidin-1-ylethyl)sulfanyl]-4- | 671.30 |
| | (trifluoromethyl)phenyl}-4,5,6,7-tetrahydro-1H-pyrazolo[4,3-c]pyridin-1- | |
| | yl]propyl}piperidin-4-yl)acetamide | |
| 530 | 1-[1-(3-{3-[3-({2-[(1-Methylethyl)amino]ethyl}sulfanyl)-4- | 671.30 |
| | (trifluoromethyl)phenyl]-5-(methylsulfonyl)-4,5,6,7-tetrahydro-1H- | |
| | pyrazolo[4,3-c]pyridin-1-yl}propyl)piperidin-4-yl]pyrrolidin-2-one | |
| 531 | 1-[1-(3-{5-(Methylsulfonyl)-3-[3-{[2-(propylamino)ethyl]sulfanyl}-4- | 671.30 |
| | (trifluoromethyl)phenyl]-4,5,6,7-tetrahydro-1H-pyrazolo[4,3-c]pyridin-1- | |
| | yl}propyl)piperidin-4-yl]pyrrolidin-2-one | |
| 532 | 1-{3-[3-{[2-(3,3-Difluoropyrrolidin-1-yl)ethyl]sulfanyl}-4- | 671.30 |
| | (trifluoromethyl)phenyl]-4,5,6,7-tetrahydro-1H-pyrazolo[4,3-c]pyridin-1- | |
| | yl}-3-[4-(pyrrolidin-1-ylcarbonyl)piperidin-1-yl]propan-2-ol | |
| 533 | 4-{1-[(2S)-3-{3-[3-{[2-(4-Fluoropiperidin-1-yl)ethyl]sulfanyl}-4- | 671.31 |
| | (trifluoromethyl)phenyl]-4,5,6,7-tetrahydro-1H-pyrazolo[4,3-c]pyridin-1- | |
| | yl}-2-hydroxypropyl]piperidin-4-yl}benzonitrile | |
| 534 | 1-(4-{4-[2-Hydroxy-3-(3-{3-[(2-piperidin-1-ylethyl)sulfanyl]-4- | 671.33 |
| | (trifluoromethyl)phenyl}-4,5,6,7-tetrahydro-1H-pyrazolo[4,3-c]pyridin-1- | |
| | yl)propyl]piperazin-1-yl}phenyl)ethanone | |
| 535 | N,N-Dimethyl-1-(1-{3-[5-(methylsulfonyl)-3-{3-[(2-piperidin-1- | 671.33 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-3-yl)methanamine | |
| 536 | 3-{1-[(2S)-3-{3-[3-{[2-(4-Fluoropiperidin-1-yl)ethyl]sulfanyl}-4- | 672.10 |
| | (trifluoromethyl)phenyl]-4,5,6,7-tetrahydro-1H-pyrazolo[4,3-c]pyridin-1- | |
| | yl}-2-hydroxypropyl]piperidin-4-yl}benzonitrile | |
| 537 | 2-[4-(3-{3-[3-{[2-(4-Fluoropiperidin-1-yl)ethyl]sulfanyl}-4- | 672.30 |
| | (trifluoromethyl)phenyl]-4,5,6,7-tetrahydro-1H-pyrazolo[4,3-c]pyridin-1- | |
| | yl}-2-hydroxypropyl)piperazin-1-yl]benzonitrile | |
| 538 | 2-{4-[(2S)-3-{3-[3-{[2-(4-Fluoropiperidin-1-yl)ethyl]sulfanyl}-4- | 672.30 |
| | (trifluoromethyl)phenyl]-4,5,6,7-tetrahydro-1H-pyrazolo[4,3-c]pyridin-1- | |
| | yl}-2-hydroxypropyl]piperazin-1-yl}benzonitrile | |
| 539 | 1-[4-(1,1-Dimethylethyl)piperidin-1-yl]-3-[5-(methylsulfonyl)-3-{3-[(2- | 672.32 |
| | pyrrolidin-1-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 540 | (2S)-1-(4,4-Dimethylpiperidin-1-yl)-3-{3-[3-{[2-(4-methylpiperidin-1- | 672.32 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 541 | 1-{4-[2-(Methyloxy)phenyl]piperidin-1-yl}-3-(5-methyl-3-{3-[(2-piperidin-1- | 672.35 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 542 | 1-(4-Cyclopropylpiperazin-1-yl)-3-[5-(methylsulfonyl)-3-{3-[(2-morpholin- | 673.28 |
| | 4-ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 543 | 1-(4-Acetylpiperazin-1-yl)-3-[5-(methylsulfonyl)-3-{3-[(2-piperidin-1- | 673.30 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 544 | 2-{4-[3-(5-Ethyl-3-{3-[(2-oxo-2-pyrrolidin-1-ylethyl)sulfanyl]-4- | 673.31 |
| | (trifluoromethyl)phenyl}-4,5,6,7-tetrahydro-1H-pyrazolo[4,3-c]pyridin-1- | |
| | yl)-2-hydroxypropyl]piperazin-1-yl}phenol | |
| 545 | 1-[4-(1-Methylethyl)piperazin-1-yl]-3-[5-(methylsulfonyl)-3-{3-[(2- | 673.31 |
| | piperidin-1-ylethyl)sulfanyl]-4-(trifluoromethyl)phenyl}-4,5,6,7-tetrahydro- | |
| | 1H-pyrazolo[4,3-c]pyridin-1-yl]propan-2-ol | |
| 546 | 1-{4-[2-(Ethyloxy)phenyl]piperazin-1-yl}-3-(3-{3-[(2-piperidin-1- | 673.34 |
| | ylethyl)sulfanyl]-4-(trifluoromethyl)phenyl}-4,5,6,7-tetrahydro-1H- | |
| | pyrazolo[4,3-c]pyridin-1-yl)propan-2-ol | |
| 547 | (2S)-1-{5-(Cyclopropylsulfonyl)-3-[3-{[2-(4-fluoropiperidin-1- | 674.27 |
| | yl)ethyl]sulfanyl}-4-(trifluoromethyl)phenyl]-4,5,6,7-tetrahydro-1H- | |
| | pyrazolo[4,3-c]pyridin-1-yl}-3-piperidin-1-ylpropan-2-ol | |
| 548 | 1-[4-(1-Hydroxy-1-methylethyl)piperidin-1-yl]-3-[5-(methylsulfonyl)-3-{3- | 674.29 |
| | [(2-pyrrolidin-1-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 549 | 1-[(2S)-2-Hydroxy-3-{3-[3-{[2-(4-methylpiperidin-1-yl)ethyl]sulfanyl}-4- | 674.29 |
| | (trifluoromethyl)phenyl]-5-(methylsulfonyl)-4,5,6,7-tetrahydro-1H- | |
| | pyrazolo[4,3-c]pyridin-1-yl}propyl]-4-methylpiperidin-4-ol | |
| 550 | (2S)-1-{3-[3-{[2-(3-Fluoropiperidin-1-yl)ethyl]sulfanyl}-4- | 674.30 |
| | (trifluoromethyl)phenyl]-5-(methylsulfonyl)-4,5,6,7-tetrahydro-1H- | |
| | pyrazolo[4,3-c]pyridin-1-yl}-3-(hexahydrocyclopenta[c]pyrrol-2(1H)- | |
| | yl)propan-2-ol | |
| 551 | N-(1-{2-Hydroxy-3-[5-(methylsulfonyl)-3-{3-[(2-morpholin-4- | 675.20 |
| | ylethyl)sulfanyl]-4-(trifluoromethyl)phenyl}-4,5,6,7-tetrahydro-1H- | |
| | pyrazolo[4,3-c]pyridin-1-yl]propyl}pyrrolidin-3-yl)acetamide | |
| 552 | 1-(4-Acetylpiperazin-1-yl)-3-[5-(methylsulfonyl)-3-{3-[(2-morpholin-4- | 675.20 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 553 | 1-[4-(Dimethylamino)piperidin-1-yl]-3-[5-(methylsulfonyl)-3-{3-[(2- | 675.29 |
| | morpholin-4-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 554 | 1-[4-(1-Methylethyl)piperazin-1-yl]-3-[5-(methylsulfonyl)-3-{3-[(2- | 675.29 |
| | morpholin-4-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 555 | 1-{2-Hydroxy-3-[5-(methylsulfonyl)-3-{3-[(2-morpholin-4-ylethyl)sulfanyl]- | 675.30 |
| | 4-(trifluoromethyl)phenyl}-4,5,6,7-tetrahydro-1H-pyrazolo[4,3-c]pyridin-1- | |
| | yl]propyl}piperidine-4-carboxamide | |
| 556 | 1-(5-Ethyl-3-{3-[(2-oxo-2-pyrrolidin-1-ylethyl)sulfanyl]-4- | 675.30 |
| | (trifluoromethyl)phenyl}-4,5,6,7-tetrahydro-1H-pyrazolo[4,3-c]pyridin-1- | |
| | yl)-3-[4-(4-fluorophenyl)piperazin-1-yl]propan-2-ol | |
| 557 | (2S)-1-{3-[3-{[2-(4-Fluoropiperidin-1-yl)ethyl]sulfanyl}-4- | 676.29 |
| | (trifluoromethyl)phenyl]-5-(propylsulfonyl)-4,5,6,7-tetrahydro-1H- | |
| | pyrazolo[4,3-c]pyridin-1-yl}-3-piperidin-1-ylpropan-2-ol | |
| 558 | (2S)-1-{3-[3-{[2-(4-Fluoropiperidin-1-yl)ethyl]sulfanyl}-4- | 676.29 |
| | (trifluoromethyl)phenyl]-5-[(1-methylethyl)sulfonyl]-4,5,6,7-tetrahydro- | |
| | 1H-pyrazolo[4,3-c]pyridin-1-yl}-3-piperidin-1-ylpropan-2-ol | |
| 559 | (2S)-1-(4,4-Dimethylpiperidin-1-yl)-3-{3-[3-{[2-(3-fluoropiperidin-1- | 676.30 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 560 | 2-[4-(3-{3-[3-{[2-(3,3-Difluoropyrrolidin-1-yl)ethyl]sulfanyl}-4- | 676.30 |
| | (trifluoromethyl)phenyl]-4,5,6,7-tetrahydro-1H-pyrazolo[4,3-c]pyridin-1- | |
| | yl}-2-hydroxypropyl)piperazin-1-yl]benzonitrile | |
| 561 | (2R)-1-(4,4-Dimethylpiperidin-1-yl)-3-{3-[3-{[2-(4-fluoropiperidin-1- | 676.30 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 562 | 1-(4,4-Dimethylpiperidin-1-yl)-3-{3-[3-{[2-(4-fluoropiperidin-1- | 676.30 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 563 | (2S)-1-{3-[3-{[2-(4-Fluoropiperidin-1-yl)ethyl]sulfanyl}-4- | 677.10 |
| | (trifluoromethyl)phenyl]-4,5,6,7-tetrahydro-1H-pyrazolo[4,3-c]pyridin-1- | |
| | yl}-3-{4-[2-(methyloxy)phenyl]piperidin-1-yl}propan-2-ol | |
| 564 | (2S)-1-{3-[3-{[2-(4-Fluoropiperidin-1-yl)ethyl]sulfanyl}-4- | 677.10 |
| | (trifluoromethyl)phenyl]-4,5,6,7-tetrahydro-1H-pyrazolo[4,3-c]pyridin-1- | |
| | yl}-3-{4-[4-(methyloxy)phenyl]piperidin-1-yl}propan-2-ol | |
| 565 | 1-[(2S)-3-{3-[3-({2-[(3S)-3-Fluoropyrrolidin-1-yl]ethyl}sulfanyl)-4- | 677.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidine-4-carboxamide | |
| 566 | 3-[3-{[2-(4-Fluoropiperidin-1-yl)ethyl]sulfanyl}-4-(trifluoromethyl)phenyl]- | 677.28 |
| | 1-[(2S)-2-hydroxy-3-piperidin-1-ylpropyl]-N,N-dimethyl-1,4,6,7- | |
| | tetrahydro-5H-pyrazolo[4,3-c]pyridine-5-sulfonamide | |
| 567 | (2S)-1-{3-[3-{[2-(4-Fluoropiperidin-1-yl)ethyl]sulfanyl}-4- | 678.10 |
| | (trifluoromethyl)phenyl]-4,5,6,7-tetrahydro-1H-pyrazolo[4,3-c]pyridin-1- | |
| | yl}-3-{4-[2-(methyloxy)phenyl]piperazin-1-yl}propan-2-ol | |
| 568 | 1-[(2S)-3-{3-[3-{[2-(3-Fluoropiperidin-1-yl)ethyl]sulfanyl}-4- | 678.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]-4-methylpiperidin-4-ol | |
| 569 | (2S)-1-{3-[3-{[2-(4-Fluoropiperidin-1-yl)ethyl]sulfanyl}-4- | 678.30 |
| | (trifluoromethyl)phenyl]-5-[(1-methyl-1H-imidazol-2-yl)carbonyl]-4,5,6,7- | |
| | tetrahydro-1H-pyrazolo[4,3-c]pyridin-1-yl}-3-piperidin-1-ylpropan-2-ol | |
| 570 | (2S)-1-{3-[3-{[2-(4-Fluoropiperidin-1-yl)ethyl]sulfanyl}-4- | 678.30 |
| | (trifluoromethyl)phenyl]-5-(methylsulfonyl)-4,5,6,7-tetrahydro-1H- | |
| | pyrazolo[4,3-c]pyridin-1-yl}-3-[2-(hydroxymethyl)piperidin-1-yl]propan-2- | |
| | ol | |
| 571 | 1-{3-[3-{[2-(4-Fluoropiperidin-1-yl)ethyl]sulfanyl}-4- | 678.40 |
| | (trifluoromethyl)phenyl]-5-(methylsulfonyl)-4,5,6,7-tetrahydro-1H- | |
| | pyrazolo[4,3-c]pyridin-1-yl}-3-[2-(hydroxymethyl)piperidin-1-yl]propan-2- | |
| | ol | |
| 572 | 1-(5-Acetyl-3-{3-[(2-morpholin-4-yl-2-oxoethyl)sulfanyl]-4- | 679.30 |
| | (trifluoromethyl)phenyl}-4,5,6,7-tetrahydro-1H-pyrazolo[4,3-c]pyridin-1- | |
| | yl)-3-(4-pyrrolidin-1-ylpiperidin-1-yl)propan-2-ol | |
| 573 | 1-(3,4-Dihydroisoquinolin-2(1H)-yl)-3-[5-(methylsulfonyl)-3-{3-[(2- | 680.20 |
| | morpholin-4-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 574 | (2S)-1-Azepan-1-yl-3-{3-[3-{[2-(3,3-difluoropiperidin-1-yl)ethyl]sulfanyl}- | 680.20 |
| | 4-(trifluoromethyl)phenyl]-5-(methylsulfonyl)-4,5,6,7-tetrahydro-1H- | |
| | pyrazolo[4,3-c]pyridin-1-yl}propan-2-ol | |
| 575 | (2S)-1-Azepan-1-yl-3-{3-[3-{[2-(4,4-difluoropiperidin-1-yl)ethyl]sulfanyl}- | 680.20 |
| | 4-(trifluoromethyl)phenyl]-5-(methylsulfonyl)-4,5,6,7-tetrahydro-1H- | |
| | pyrazolo[4,3-c]pyridin-1-yl}propan-2-ol | |
| 576 | (2S)-1-{3-[3-{[2-(4,4-Difluoropiperidin-1-yl)ethyl]sulfanyl}-4- | 680.20 |
| | (trifluoromethyl)phenyl]-5-(methylsulfonyl)-4,5,6,7-tetrahydro-1H- | |
| | pyrazolo[4,3-c]pyridin-1-yl}-3-(4-methylpiperidin-1-yl)propan-2-ol | |
| 577 | (2S)-1-{3-[3-{[2-(4-Fluoropiperidin-1-yl)ethyl]sulfanyl}-4- | 680.26 |
| | (trifluoromethyl)phenyl]-5-(2-thienylcarbonyl)-4,5,6,7-tetrahydro-1H- | |
| | pyrazolo[4,3-c]pyridin-1-yl}-3-piperidin-1-ylpropan-2-ol | |
| 578 | 1-{3-[3-{[2-(4-Fluoropiperidin-1-yl)ethyl]sulfanyl}-4- | 680.26 |
| | (trifluoromethyl)phenyl]-5-(2-thienylcarbonyl)-4,5,6,7-tetrahydro-1H- | |
| | pyrazolo[4,3-c]pyridin-1-yl}-3-piperidin-1-ylpropan-2-ol | |
| 579 | (2S)-1-[4-(3-Chlorophenyl)piperidin-1-yl]-3-{3-[3-{[2-(4-fluoropiperidin-1- | 680.27 |
| | yl)ethyl]sulfanyl}-4-(trifluoromethyl)phenyl]-4,5,6,7-tetrahydro-1H- | |
| | pyrazolo[4,3-c]pyridin-1-yl}propan-2-ol | |
| 580 | N-[1-(3-{5-(Methylsulfonyl)-3-[3-{[2-(propylamino)ethyl]sulfanyl}-4- | 681.20 |
| | (trifluoromethyl)phenyl]-4,5,6,7-tetrahydro-1H-pyrazolo[4,3-c]pyridin-1- | |
| | yl}propyl)piperidin-4-yl]methanesulfonamide | |
| 581 | 1-{3-[3-{[2-(3,3-Difluoropyrrolidin-1-yl)ethyl]sulfanyl}-4- | 681.30 |
| | (trifluoromethyl)phenyl]-4,5,6,7-tetrahydro-1H-pyrazolo[4,3-c]pyridin-1- | |
| | yl}-3-{4-[3-(methyloxy)phenyl]piperazin-1-yl}propan-2-ol | |
| 582 | 1-{3-[3-{[2-(4-Fluoropiperidin-1-yl)ethyl]sulfanyl}-4- | 681.30 |
| | (trifluoromethyl)phenyl]-5-(1-methyl-L-prolyl)-4,5,6,7-tetrahydro-1H- | |
| | pyrazolo[4,3-c]pyridin-1-yl}-3-piperidin-1-ylpropan-2-ol | |
| 583 | 1-{3-[3-{[2-(3,3-Difluoropyrrolidin-1-yl)ethyl]sulfanyl}-4- | 681.30 |
| | (trifluoromethyl)phenyl]-4,5,6,7-tetrahydro-1H-pyrazolo[4,3-c]pyridin-1- | |
| | yl}-3-{4-[4-(methyloxy)phenyl]piperazin-1-yl}propan-2-ol | |
| 584 | 1-{3-[3-{[2-(4-Fluoropiperidin-1-yl)ethyl]sulfanyl}-4- | 681.30 |
| | (trifluoromethyl)phenyl]-5-(piperidin-3-ylcarbonyl)-4,5,6,7-tetrahydro-1H- | |
| | pyrazolo[4,3-c]pyridin-1-yl}-3-piperidin-1-ylpropan-2-ol | |
| 585 | 4-{1-[3-(5-Ethyl-3-{3-[(2-oxo-2-pyrrolidin-1-ylethyl)sulfanyl]-4- | 681.31 |
| | (trifluoromethyl)phenyl}-4,5,6,7-tetrahydro-1H-pyrazolo[4,3-c]pyridin-1- | |
| | yl)-2-hydroxypropyl]piperidin-4-yl}benzonitrile | |
| 586 | 2-Hydroxy-N-{1-[2-hydroxy-3-(5-[(methyloxy)acetyl]-3-{3-[(2-pyrrolidin-1- | 683.31 |
| | ylethyl)sulfanyl]-4-(trifluoromethyl)phenyl}-4,5,6,7-tetrahydro-1H- | |
| | pyrazolo[4,3-c]pyridin-1-yl)propyl]piperidin-4-yl}acetamide | |
| 587 | (2S)-1-{3-[3-{[2-(4-Fluoropiperidin-1-yl)ethyl]sulfanyl}-4- | 683.33 |
| | (trifluoromethyl)phenyl]-5-(morpholin-4-ylcarbonyl)-4,5,6,7-tetrahydro- | |
| | 1H-pyrazolo[4,3-c]pyridin-1-yl}-3-piperidin-1-ylpropan-2-ol | |
| 588 | (2S)-1-{3-[3-{[2-(3,3-Difluoropiperidin-1-yl)ethyl]sulfanyl}-4- | 684.20 |
| | (trifluoromethyl)phenyl]-5-(methylsulfonyl)-4,5,6,7-tetrahydro-1H- | |
| | pyrazolo[4,3-c]pyridin-1-yl}-3-thiomorpholin-4-ylpropan-2-ol | |
| 589 | (2S)-1-{3-[3-{[2-(4,4-Difluoropiperidin-1-yl)ethyl]sulfanyl}-4- | 684.20 |
| | (trifluoromethyl)phenyl]-5-(methylsulfonyl)-4,5,6,7-tetrahydro-1H- | |
| | pyrazolo[4,3-c]pyridin-1-yl}-3-thiomorpholin-4-ylpropan-2-ol | |
| 590 | (2S)-1-(4,4-Difluoropiperidin-1-yl)-3-{3-[3-{[2-(4-fluoropiperidin-1- | 684.20 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 591 | (2S)-1-{5-(Methylsulfonyl)-3-[3-{[2-(octahydroisoquinolin-2(1H)- | 684.32 |
| | yl)ethyl]sulfanyl}-4-(trifluoromethyl)phenyl]-4,5,6,7-tetrahydro-1H- | |
| | pyrazolo[4,3-c]pyridin-1-yl}-3-piperidin-1-ylpropan-2-ol | |
| 592 | 1-[1-(3-{3-[3-({2-[(2-Methylpropyl)amino]ethyl}sulfanyl)-4- | 685.30 |
| | (trifluoromethyl)phenyl]-5-(methylsulfonyl)-4,5,6,7-tetrahydro-1H- | |
| | pyrazolo[4,3-c]pyridin-1-yl}propyl)piperidin-4-yl]pyrrolidin-2-one | |
| 593 | 1-{5-(Methylsulfonyl)-3-[3-{[2-(4-pyrrolidin-1-ylpiperidin-1- | 685.30 |
| | yl)ethyl]sulfanyl}-4-(trifluoromethyl)phenyl]-4,5,6,7-tetrahydro-1H- | |
| | pyrazolo[4,3-c]pyridin-1-yl}-3-pyrrolidin-1-ylpropan-2-ol | |
| 594 | (2S)-1-{5-(Methylsulfonyl)-3-[3-{[2-(octahydroisoquinolin-2(1H)- | 686.29 |
| | yl)ethyl]sulfanyl}-4-(trifluoromethyl)phenyl]-4,5,6,7-tetrahydro-1H- | |
| | pyrazolo[4,3-c]pyridin-1-yl}-3-morpholin-4-ylpropan-2-ol | |
| 595 | 1-[5-(Methylsulfonyl)-3-{3-[(2-morpholin-4-ylethyl)sulfanyl]-4- | 686.30 |
| | (trifluoromethyl)phenyl}-4,5,6,7-tetrahydro-1H-pyrazolo[4,3-c]pyridin-1- | |
| | yl]-3-(octahydroisoquinolin-2(1H)-yl)propan-2-ol | |
| 596 | N-[(1-{2-Hydroxy-3-[5-(methylsulfonyl)-3-{3-[(2-pyrrolidin-1- | 687.29 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)methyl]acetamide | |
| 597 | 1-[(2S)-2-Hydroxy-3-{3-[3-{[2-(4-methylpiperidin-1-yl)ethyl]sulfanyl}-4- | 687.29 |
| | (trifluoromethyl)phenyl]-5-(methylsulfonyl)-4,5,6,7-tetrahydro-1H- | |
| | pyrazolo[4,3-c]pyridin-1-yl}propyl]piperidine-4-carboxamide | |
| 598 | 1-[1-(3-{3-[3-({2-[(3-Hydroxypropyl)amino]ethyl}sulfanyl)-4- | 687.30 |
| | (trifluoromethyl)phenyl]-5-(methylsulfonyl)-4,5,6,7-tetrahydro-1H- | |
| | pyrazolo[4,3-c]pyridin-1-yl}propyl)piperidin-4-yl]pyrrolidin-2-one | |
| 599 | 1-[5-(Hydroxyacetyl)-3-{3-[(2-piperidin-1-ylethyl)sulfanyl]-4- | 687.32 |
| | (trifluoromethyl)phenyl}-4,5,6,7-tetrahydro-1H-pyrazolo[4,3-c]pyridin-1- | |
| | yl]-3-(4-pyridin-2-ylpiperidin-1-yl)propan-2-ol | |
| 600 | 1-(1-{2-Hydroxy-3-[5-(methylsulfonyl)-3-{3-[(2-piperidin-1- | 688.28 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)urea | |
| 601 | 1-(1,5-Dioxa-9-azaspiro[5.5]undec-9-yl)-3-[5-(methylsulfonyl)-3-{3-[(2- | 688.30 |
| | pyrrolidin-1-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 602 | (2S)-1-{3-[3-({2-[4-(1-Hydroxy-1-methylethyl)piperidin-1- | 688.30 |
| | yl]ethyl}sulfanyl)-4-(trifluoromethyl)phenyl]-5-(methylsulfonyl)-4,5,6,7- | |
| | tetrahydro-1H-pyrazolo[4,3-c]pyridin-1-yl}-3-piperidin-1-ylpropan-2-ol | |
| 603 | 2-Hydroxy-N-(1-{2-hydroxy-3-[5-(methylsulfonyl)-3-{3-[(2-pyrrolidin-1- | 689.27 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)acetamide | |
| 604 | 3-[3-{[2-(4-Fluoropiperidin-1-yl)ethyl]sulfanyl}-4-(trifluoromethyl)phenyl]- | 689.32 |
| | 1-[(2S)-2-hydroxy-3-piperidin-1-ylpropyl]-N-phenyl-1,4,6,7-tetrahydro- | |
| | 5H-pyrazolo[4,3-c]pyridine-5-carboxamide | |
| 605 | (2S)-1-[4-(1,1-Dimethylethyl)piperidin-1-yl]-3-{3-[3-({2-[(3S)-3- | 690.30 |
| | fluoropyrrolidin-1-yl]ethyl}sulfanyl)-4-(trifluoromethyl)phenyl]-5- | |
| | (methylsulfonyl)-4,5,6,7-tetrahydro-1H-pyrazolo[4,3-c]pyridin-1- | |
| | yl}propan-2-ol | |
| 606 | N-{1-[(2S)-3-{3-[3-({2-[(3S)-3-Fluoropyrrolidin-1-yl]ethyl}sulfanyl)-4- | 691.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-4-yl}acetamide | |
| 607 | (2S)-1-[4-(Dimethylamino)piperidin-1-yl]-3-{3-[3-{[2-(4-fluoropiperidin-1- | 691.30 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 608 | 1-[(2S)-3-{3-[3-{[2-(3-Fluoropiperidin-1-yl)ethyl]sulfanyl}-4- | 691.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidine-4-carboxamide | |
| 609 | (2S)-1-{4-[2-(Ethyloxy)phenyl]piperazin-1-yl}-3-{3-[3-{[2-(4- | 692.10 |
| | fluoropiperidin-1-yl)ethyl]sulfanyl}-4-(trifluoromethyl)phenyl]-4,5,6,7- | |
| | tetrahydro-1H-pyrazolo[4,3-c]pyridin-1-yl}propan-2-ol | |
| 610 | 1-(3-{3-[3-{[2-(4-Fluoropiperidin-1-yl)ethyl]sulfanyl}-4- | 692.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidine-4-carboxylic acid | |
| 611 | (3S)-1-(3-{3-[3-{[2-(4-Fluoropiperidin-1-yl)ethyl]sulfanyl}-4- | 692.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidine-3-carboxylic acid | |
| 612 | (2S)-1-{3-[3-{[2-(4,4-Difluoropiperidin-1-yl)ethyl]sulfanyl}-4- | 692.20 |
| | (trifluoromethyl)phenyl]-5-(methylsulfonyl)-4,5,6,7-tetrahydro-1H- | |
| | pyrazolo[4,3-c]pyridin-1-yl}-3-(hexahydrocyclopenta[c]pyrrol-2(1H)- | |
| | yl)propan-2-ol | |
| 613 | (3R)-1-(3-{3-[3-{[2-(4-Fluoropiperidin-1-yl)ethyl]sulfanyl}-4- | 692.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidine-3-carboxylic acid | |
| 614 | (3R)-1-[(2S)-3-{3-[3-{[2-(4-Fluoropiperidin-1-yl)ethyl]sulfanyl}-4- | 692.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidine-3-carboxylic acid | |
| 615 | (2S)-1-{3-[3-({2-[(3S)-3-Fluoropyrrolidin-1-yl]ethyl}sulfanyl)-4- | 692.20 |
| | (trifluoromethyl)phenyl]-5-(methylsulfonyl)-4,5,6,7-tetrahydro-1H- | |
| | pyrazolo[4,3-c]pyridin-1-yl}-3-[4-(1-hydroxy-1-methylethyl)piperidin-1- | |
| | yl]propan-2-ol | |
| 616 | 1-[5-(Methylsulfonyl)-3-{3-[(2-pyrrolidin-1-ylethyl)sulfanyl]-4- | 692.28 |
| | (trifluoromethyl)phenyl}-4,5,6,7-tetrahydro-1H-pyrazolo[4,3-c]pyridin-1- | |
| | yl]-3-(4-phenylpiperidin-1-yl)propan-2-ol | |
| 617 | 1-[(2S)-3-{3-[3-{[2-(4-Fluoropiperidin-1-yl)ethyl]sulfanyl}-4- | 692.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidine-3-carboxylic acid | |
| 618 | (2S)-1-{3-[3-{[2-(3,3-Difluoropiperidin-1-yl)ethyl]sulfanyl}-4- | 692.30 |
| | (trifluoromethyl)phenyl]-5-(methylsulfonyl)-4,5,6,7-tetrahydro-1H- | |
| | pyrazolo[4,3-c]pyridin-1-yl}-3-(hexahydrocyclopenta[c]pyrrol-2(1H)- | |
| | yl)propan-2-ol | |
| 619 | N-[1-(3-{3-[3-{[2-(4-Fluoropiperidin-1-yl)ethyl]sulfanyl}-4- | 692.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)pyrrolidin-3-yl]acetamide | |
| 620 | (2S)-1-{3-[3-{[2-(4-Fluoropiperidin-1-yl)ethyl]sulfanyl}-4- | 692.30 |
| | (trifluoromethyl)phenyl]-5-(methylsulfonyl)-4,5,6,7-tetrahydro-1H- | |
| | pyrazolo[4,3-c]pyridin-1-yl}-3-[2-(2-hydroxyethyl)piperidin-1-yl]propan-2- | |
| | ol | |
| 621 | 1-[5-(Methylsulfonyl)-3-{3-[(2-pyrrolidin-1-ylethyl)sulfanyl]-4- | 693.28 |
| | (trifluoromethyl)phenyl}-4,5,6,7-tetrahydro-1H-pyrazolo[4,3-c]pyridin-1- | |
| | yl]-3-(4-pyridin-2-ylpiperidin-1-yl)propan-2-ol | |
| 622 | (2S)-1-{3-[3-{[2-(4,4-Difluoropiperidin-1-yl)ethyl]sulfanyl}-4- | 693.30 |
| | (trifluoromethyl)phenyl]-5-(methylsulfonyl)-4,5,6,7-tetrahydro-1H- | |
| | pyrazolo[4,3-c]pyridin-1-yl}-3-(4,4-dimethylpiperidin-1-yl)propan-2-ol | |
| 623 | (2S)-1-{3-[3-{[2-(3,3-Difluoropiperidin-1-yl)ethyl]sulfanyl}-4- | 694.30 |
| | (trifluoromethyl)phenyl]-5-(methylsulfonyl)-4,5,6,7-tetrahydro-1H- | |
| | pyrazolo[4,3-c]pyridin-1-yl}-3-(4,4-dimethylpiperidin-1-yl)propan-2-ol | |
| 624 | N-[1-(3-{3-[3-{[2-(3,3-Difluoropyrrolidin-1-yl)ethyl]sulfanyl}-4- | 695.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)pyrrolidin-3-yl]acetamide | |
| 625 | 1-(3-{3-[3-{[2-(3,3-Difluoropyrrolidin-1-yl)ethyl]sulfanyl}-4- | 695.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidine-4-carboxamide | |
| 626 | 3-[3-{[2-(4-Fluoropiperidin-1-yl)ethyl]sulfanyl}-4-(trifluoromethyl)phenyl]- | 695.28 |
| | 1-[(2S)-2-hydroxy-3-piperidin-1-ylpropyl]-N-2-thienyl-1,4,6,7-tetrahydro- | |
| | 5H-pyrazolo[4,3-c]pyridine-5-carboxamide | |
| 627 | 1-{3-[3-{[2-(4-Fluoropiperidin-1-yl)ethyl]sulfanyl}-4- | 695.30 |
| | (trifluoromethyl)phenyl]-5-(piperidin-4-ylacetyl)-4,5,6,7-tetrahydro-1H- | |
| | pyrazolo[4,3-c]pyridin-1-yl}-3-piperidin-1-ylpropan-2-ol | |
| 628 | 1-[5-(Hydroxyacetyl)-3-{3-[(2-morpholin-4-yl-2-oxoethyl)sulfanyl]-4- | 695.30 |
| | (trifluoromethyl)phenyl}-4,5,6,7-tetrahydro-1H-pyrazolo[4,3-c]pyridin-1- | |
| | yl]-3-(4-pyrrolidin-1-ylpiperidin-1-yl)propan-2-ol | |
| 629 | 1-{3-[3-{[2-(3,3-Difluoropyrrolidin-1-yl)ethyl]sulfanyl}-4- | 695.30 |
| | (trifluoromethyl)phenyl]-4,5,6,7-tetrahydro-1H-pyrazolo[4,3-c]pyridin-1- | |
| | yl}-3-{4-[2-(ethyloxy)phenyl]piperazin-1-yl}propan-2-ol | |
| 630 | N-Cyclohexyl-3-[3-{[2-(4-fluoropiperidin-1-yl)ethyl]sulfanyl}-4- | 695.36 |
| | (trifluoromethyl)phenyl]-1-[(2S)-2-hydroxy-3-piperidin-1-ylpropyl]-1,4,6,7- | |
| | tetrahydro-5H-pyrazolo[4,3-c]pyridine-5-carboxamide | |
| 631 | 1-[(2S)-3-{3-[3-{[2-(3,3-Difluoropiperidin-1-yl)ethyl]sulfanyl}-4- | 696.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]-4-methylpiperidin-4-ol | |
| 632 | 1-[(2S)-3-{3-[3-{[2-(4,4-Difluoropiperidin-1-yl)ethyl]sulfanyl}-4- | 696.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]-4-methylpiperidin-4-ol | |
| 633 | (2S)-1-{3-[3-{[2-(4-Fluoropiperidin-1-yl)ethyl]sulfanyl}-4- | 696.30 |
| | (trifluoromethyl)phenyl]-5-[(4-methylpiperazin-1-yl)carbonyl]-4,5,6,7- | |
| | tetrahydro-1H-pyrazolo[4,3-c]pyridin-1-yl}-3-piperidin-1-ylpropan-2-ol | |
| 634 | 1-(3-{3-[(2-Piperidin-1-ylethyl)sulfanyl]-4-(trifluoromethyl)phenyl}-4,5,6,7- | 697.30 |
| | tetrahydro-1H-pyrazolo[4,3-c]pyridin-1-yl)-3-{4-[3- | |
| | (trifluoromethyl)phenyl]piperazin-1-yl}propan-2-ol | |
| 635 | 3-[3-{[2-(4-Fluoropiperidin-1-yl)ethyl]sulfanyl}-4-(trifluoromethyl)phenyl]- | 697.38 |
| | 1-[(2S)-2-hydroxy-3-piperidin-1-ylpropyl]-N,N-bis(1-methylethyl)-1,4,6,7- | |
| | tetrahydro-5H-pyrazolo[4,3-c]pyridine-5-carboxamide | |
| 636 | (2S)-1-[4-(1,1-Difluoroethyl)piperidin-1-yl]-3-{3-[3-({2-[(3S)-3- | 698.20 |
| | fluoropyrrolidin-1-yl]ethyl}sulfanyl)-4-(trifluoromethyl)phenyl]-5- | |
| | (methylsulfonyl)-4,5,6,7-tetrahydro-1H-pyrazolo[4,3-c]pyridin-1- | |
| | yl}propan-2-ol | |
| 637 | (2S)-1-(4-Methylpiperidin-1-yl)-3-{5-(methylsulfonyl)-3-[3-{[2- | 698.33 |
| | (octahydroisoquinolin-2(1H)-yl)ethyl]sulfanyl}-4-(trifluoromethyl)phenyl]- | |
| | 4,5,6,7-tetrahydro-1H-pyrazolo[4,3-c]pyridin-1-yl}propan-2-ol | |
| 638 | 2-Hydroxy-N-{1-[2-hydroxy-3-(5-[(methylsulfanyl)acetyl]-3-{3-[(2- | 699.29 |
| | pyrrolidin-1-ylethyl)sulfanyl]-4-(trifluoromethyl)phenyl}-4,5,6,7- | |
| | tetrahydro-1H-pyrazolo[4,3-c]pyridin-1-yl)propyl]piperidin-4-yl}acetamide | |
| 639 | 1-(1-{2-Hydroxy-3-[5-(methylsulfonyl)-3-{3-[(2-pyrrolidin-1- | 699.29 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)pyrrolidin-2-one | |
| 640 | 1-(1,4′-Bipiperidin-1′-yl)-3-[5-(methylsulfonyl)-3-{3-[(2-pyrrolidin-1- | 699.33 |
| | ylethyl)sulfanyl]-4-(trifluoromethyl)phenyl}-4,5,6,7-tetrahydro-1H- | |
| | pyrazolo[4,3-c]pyridin-1-yl]propan-2-ol | |
| 641 | 1-[5-(Methylsulfonyl)-3-{3-[(2-piperidin-1-ylethyl)sulfanyl]-4- | 699.33 |
| | (trifluoromethyl)phenyl}-4,5,6,7-tetrahydro-1H-pyrazolo[4,3-c]pyridin-1- | |
| | yl]-3-(4-pyrrolidin-1-ylpiperidin-1-yl)propan-2-ol | |
| 642 | 1-[5-(Methylsulfonyl)-3-{3-[(2-morpholin-4-ylethyl)sulfanyl]-4- | 700.20 |
| | (trifluoromethyl)phenyl}-4,5,6,7-tetrahydro-1H-pyrazolo[4,3-c]pyridin-1- | |
| | yl]-3-[4-(trifluoromethyl)piperidin-1-yl]propan-2-ol | |
| 643 | (2S)-1-[4-(1,1-Dimethylethyl)piperidin-1-yl]-3-{3-[3-{[2-(4-methylpiperidin- | 700.35 |
| | 1-yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 644 | 1-[5-(Methylsulfonyl)-3-{3-[(2-morpholin-4-ylethyl)sulfanyl]-4- | 701.30 |
| | (trifluoromethyl)phenyl}-4,5,6,7-tetrahydro-1H-pyrazolo[4,3-c]pyridin-1- | |
| | yl]-3-(4-pyrrolidin-1-ylpiperidin-1-yl)propan-2-ol | |
| 645 | N-{1-[(2S)-2-Hydroxy-3-{3-[3-{[2-(4-methylpiperidin-1-yl)ethyl]sulfanyl}-4- | 701.30 |
| | (trifluoromethyl)phenyl]-5-(methylsulfonyl)-4,5,6,7-tetrahydro-1H- | |
| | pyrazolo[4,3-c]pyridin-1-yl}propyl]piperidin-4-yl}acetamide | |
| 646 | 1-[5-(Methylsulfonyl)-3-{3-[(2-pyrrolidin-1-ylethyl)sulfanyl]-4- | 701.30 |
| | (trifluoromethyl)phenyl}-4,5,6,7-tetrahydro-1H-pyrazolo[4,3-c]pyridin-1- | |
| | yl]-3-(4-morpholin-4-ylpiperidin-1-yl)propan-2-ol | |
| 647 | (2S)-1-{3-[3-{[2-(4-Fluoropiperidin-1-yl)ethyl]sulfanyl}-4- | 702.23 |
| | (trifluoromethyl)phenyl]-5-[(trifluoromethyl)sulfonyl]-4,5,6,7-tetrahydro- | |
| | 1H-pyrazolo[4,3-c]pyridin-1-yl}-3-piperidin-1-ylpropan-2-ol | |
| 648 | (2S)-1-{3-[3-{[2-(4-Fluoropiperidin-1-yl)ethyl]sulfanyl}-4- | 702.30 |
| | (trifluoromethyl)phenyl]-5-(methylsulfonyl)-4,5,6,7-tetrahydro-1H- | |
| | pyrazolo[4,3-c]pyridin-1-yl}-3-(octahydroquinolin-1(2H)-yl)propan-2-ol | |
| 649 | (2S)-1-[4-(1-Hydroxy-1-methylethyl)piperidin-1-yl]-3-{3-[3-{[2-(4- | 702.33 |
| | methylpiperidin-1-yl)ethyl]sulfanyl}-4-(trifluoromethyl)phenyl]-5- | |
| | (methylsulfonyl)-4,5,6,7-tetrahydro-1H-pyrazolo[4,3-c]pyridin-1- | |
| | yl}propan-2-ol | |
| 650 | N-[(1-{2-Hydroxy-3-[5-(methylsulfonyl)-3-{3-[(2-morpholin-4- | 703.20 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)methyl]acetamide | |
| 651 | Ethyl (1-{2-hydroxy-3-[5-(methylsulfonyl)-3-{3-[(2-pyrrolidin-1- | 703.28 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)carbamate | |
| 652 | (2S)-1-[4-(1,1-Dimethylethyl)piperidin-1-yl]-3-{3-[3-{[2-(3-fluoropiperidin- | 704.30 |
| | 1-yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 653 | 1-{3-[3-{[2-(4-Fluoropiperidin-1-yl)ethyl]sulfanyl}-4- | 704.32 |
| | (trifluoromethyl)phenyl]-5-[(2S)-2-hydroxy-2-phenylacetyl]-4,5,6,7- | |
| | tetrahydro-1H-pyrazolo[4,3-c]pyridin-1-yl}-3-piperidin-1-ylpropan-2-ol | |
| 654 | 2-Hydroxy-N-[1-(2-{[5-{1-[(2S)-2-hydroxy-3-morpholin-4-ylpropyl]-5- | 705.20 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)piperidin-4-yl]acetamide | |
| 655 | N-{1-[(2S)-3-{3-[3-{[2-(3-Fluoropiperidin-1-yl)ethyl]sulfanyl}-4- | 705.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-4-yl}acetamide | |
| 656 | (2S)-1-{3-[3-{[2-(3-Fluoropiperidin-1-yl)ethyl]sulfanyl}-4- | 706.30 |
| | (trifluoromethyl)phenyl]-5-(methylsulfonyl)-4,5,6,7-tetrahydro-1H- | |
| | pyrazolo[4,3-c]pyridin-1-yl}-3-[4-(1-hydroxy-1-methylethyl)piperidin-1- | |
| | yl]propan-2-ol | |
| 657 | 2-Hydroxy-N-(1-{2-hydroxy-3-[5-(methylsulfonyl)-3-{3-[(2-morpholin-4- | 706.30 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)acetamide | |
| 658 | N-{1-[(2S)-3-{3-[3-({2-[(3S)-3-Fluoropyrrolidin-1-yl]ethyl}sulfanyl)-4- | 707.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-4-yl}-2- | |
| | hydroxyacetamide | |
| 659 | 1-[5-(Methylsulfonyl)-3-{3-[(2-piperidin-1-ylethyl)sulfanyl]-4- | 707.29 |
| | (trifluoromethyl)phenyl}-4,5,6,7-tetrahydro-1H-pyrazolo[4,3-c]pyridin-1- | |
| | yl]-3-(4-phenylpiperazin-1-yl)propan-2-ol | |
| 660 | 1-[5-(Methylsulfonyl)-3-{3-[(2-piperidin-1-ylethyl)sulfanyl]-4- | 707.29 |
| | (trifluoromethyl)phenyl}-4,5,6,7-tetrahydro-1H-pyrazolo[4,3-c]pyridin-1- | |
| | yl]-3-(4-pyridin-4-ylpiperidin-1-yl)propan-2-ol | |
| 661 | 1-(5-Acetyl-3-{3-[(2-morpholin-4-yl-2-oxoethyl)sulfanyl]-4- | 707.30 |
| | (trifluoromethyl)phenyl}-4,5,6,7-tetrahydro-1H-pyrazolo[4,3-c]pyridin-1- | |
| | yl)-3-[4-(pyrrolidin-1-ylcarbonyl)piperidin-1-yl]propan-2-ol | |
| 662 | 1-[5-(Methylsulfonyl)-3-{3-[(2-morpholin-4-ylethyl)sulfanyl]-4- | 708.30 |
| | (trifluoromethyl)phenyl}-4,5,6,7-tetrahydro-1H-pyrazolo[4,3-c]pyridin-1- | |
| | yl]-3-(4-phenylpiperidin-1-yl)propan-2-ol | |
| 663 | 2-(1-[(2S)-2-Hydroxy-3-{4-[(4R)-4-hydroxy-2-oxopyrrolidin-1-yl]piperidin- | 708.31 |
| | 1-yl}propyl]-3-{3-[(2-pyrrolidin-1-ylethyl)sulfanyl]-4- | |
| | (trifluoromethyl)phenyl}-1,4,6,7-tetrahydro-5H-pyrazolo[4,3-c]pyridin-5- | |
| | yl)-2-oxoacetamide | |
| 664 | 1-[5-(Methylsulfonyl)-3-{3-[(2-morpholin-4-ylethyl)sulfanyl]-4- | 709.20 |
| | (trifluoromethyl)phenyl}-4,5,6,7-tetrahydro-1H-pyrazolo[4,3-c]pyridin-1- | |
| | yl]-3-(4-pyridin-4-ylpiperidin-1-yl)propan-2-ol | |
| 665 | 1-[5-(Methylsulfonyl)-3-{3-[(2-morpholin-4-ylethyl)sulfanyl]-4- | 709.20 |
| | (trifluoromethyl)phenyl}-4,5,6,7-tetrahydro-1H-pyrazolo[4,3-c]pyridin-1- | |
| | yl]-3-(4-phenylpiperazin-1-yl)propan-2-ol | |
| 666 | N-(2-{[5-{1-[2-Hydroxy-3-(4-pyrrolidin-1-ylpiperidin-1-yl)propyl]-5- | 709.20 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)methanesulfonamide | |
| 667 | 1-[(2S)-3-{3-[3-{[2-(4,4-Difluoropiperidin-1-yl)ethyl]sulfanyl}-4- | 709.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidine-4-carboxamide | |
| 668 | 1-[(2S)-3-{3-[3-{[2-(3,3-Difluoropiperidin-1-yl)ethyl]sulfanyl}-4- | 709.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidine-4-carboxamide | |
| 669 | N-(1-{2-Hydroxy-3-[5-(methylsulfonyl)-3-{3-[(2-pyrrolidin-1- | 709.24 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)methanesulfonamide | |
| 670 | 1-{2-Hydroxy-3-[5-(methylsulfonyl)-3-{3-[(2-pyrrolidin-1-ylethyl)sulfanyl]- | 709.27 |
| | 4-(trifluoromethyl)phenyl}-4,5,6,7-tetrahydro-1H-pyrazolo[4,3-c]pyridin-1- | |
| | yl]propyl}-4-pyridin-2-ylpiperidin-4-ol | |
| 671 | 1-[1-(2-Hydroxy-3-{5-(hydroxyacetyl)-3-[3-{[2-(3-hydroxypiperidin-1- | 709.30 |
| | yl)ethyl]sulfanyl}-4-(trifluoromethyl)phenyl]-4,5,6,7-tetrahydro-1H- | |
| | pyrazolo[4,3-c]pyridin-1-yl}propyl)piperidin-4-yl]pyrrolidin-2-one | |
| 672 | 1-{5-(1-Acetyl-L-prolyl)-3-[3-{[2-(4-fluoropiperidin-1-yl)ethyl]sulfanyl}-4- | 709.30 |
| | (trifluoromethyl)phenyl]-4,5,6,7-tetrahydro-1H-pyrazolo[4,3-c]pyridin-1- | |
| | yl}-3-piperidin-1-ylpropan-2-ol | |
| 673 | 1-[5-(Methylsulfonyl)-3-{3-[(2-morpholin-4-ylethyl)sulfanyl]-4- | 711.20 |
| | (trifluoromethyl)phenyl}-4,5,6,7-tetrahydro-1H-pyrazolo[4,3-c]pyridin-1- | |
| | yl]-3-(4-pyrimidin-2-ylpiperazin-1-yl)propan-2-ol | |
| 674 | 1-[1-(3-{3-[3-{[2-(Cyclohexylamino)ethyl]sulfanyl}-4- | 711.30 |
| | (trifluoromethyl)phenyl]-5-(methylsulfonyl)-4,5,6,7-tetrahydro-1H- | |
| | pyrazolo[4,3-c]pyridin-1-yl}propyl)piperidin-4-yl]pyrrolidin-2-one | |
| 675 | 1-[2-Hydroxy-3-(3-{3-[(2-piperidin-1-ylethyl)sulfanyl]-4- | 712.30 |
| | (trifluoromethyl)phenyl}-4,5,6,7-tetrahydro-1H-pyrazolo[4,3-c]pyridin-1- | |
| | yl)propyl]-4-[3-(trifluoromethyl)phenyl]piperidin-4-ol | |
| 676 | 2-(4-{2-Hydroxy-3-[5-(hydroxyacetyl)-3-{3-[(2-piperidin-1- | 712.32 |
| | ylethyl)sulfanyl]-4-(trifluoromethyl)phenyl}-4,5,6,7-tetrahydro-1H- | |
| | pyrazolo[4,3-c]pyridin-1-yl]propyl}piperazin-1-yl)benzonitrile | |
| 677 | 5-(1-{2-Hydroxy-3-[5-(methylsulfonyl)-3-{3-[(2-piperidin-1- | 713.20 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)-2,4-dihydro-3H-1,2,4- | |
| | triazol-3-one | |
| 678 | 4-(1-{2-Hydroxy-3-[5-(methylsulfonyl)-3-{3-[(2-piperidin-1- | 713.30 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)-2,4-dihydro-3H-1,2,4- | |
| | triazol-3-one | |
| 679 | 4-Hydroxy-1-(1-{3-[5-(methylsulfonyl)-3-{3-[(2-piperidin-1- | 713.30 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)pyrrolidin-2-one | |
| 680 | 1-[5-(Methylsulfonyl)-3-{3-[(2-pyrrolidin-1-ylethyl)sulfanyl]-4- | 713.30 |
| | (trifluoromethyl)phenyl}-4,5,6,7-tetrahydro-1H-pyrazolo[4,3-c]pyridin-1- | |
| | yl]-3-[4-(pyrrolidin-1-ylcarbonyl)piperidin-1-yl]propan-2-ol | |
| 681 | 1-[4-(3-Methyl-1,2,4-oxadiazol-5-yl)piperidin-1-yl]-3-[5-(methylsulfonyl)- | 714.20 |
| | 3-{3-[(2-morpholin-4-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 682 | 1-{3-[3-{[2-(4-Acetylpiperazin-1-yl)ethyl]sulfanyl}-4- | 714.30 |
| | (trifluoromethyl)phenyl]-5-(methylsulfonyl)-4,5,6,7-tetrahydro-1H- | |
| | pyrazolo[4,3-c]pyridin-1-yl}-3-(4-cyclopropylpiperazin-1-yl)propan-2-ol | |
| 683 | 5-(1-{2-Hydroxy-3-[5-(methylsulfonyl)-3-{3-[(2-morpholin-4- | 715.20 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)-2,4-dihydro-3H-1,2,4- | |
| | triazol-3-one | |
| 684 | 4-(1-{2-Hydroxy-3-[5-(methylsulfonyl)-3-{3-[(2-morpholin-4- | 715.20 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)-2,4-dihydro-3H-1,2,4- | |
| | triazol-3-one | |
| 685 | 4-Hydroxy-1-(1-{2-hydroxy-3-[5-(methylsulfonyl)-3-{3-[(2-pyrrolidin-1- | 715.28 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)pyrrolidin-2-one | |
| 686 | 1-[5-(Methylsulfonyl)-3-{3-[(2-morpholin-4-yl-2-oxoethyl)sulfanyl]-4- | 715.28 |
| | (trifluoromethyl)phenyl}-4,5,6,7-tetrahydro-1H-pyrazolo[4,3-c]pyridin-1- | |
| | yl]-3-(4-pyrrolidin-1-ylpiperidin-1-yl)propan-2-ol | |
| 687 | 1-[5-(Methylsulfonyl)-3-{3-[(2-piperidin-1-ylethyl)sulfanyl]-4- | 715.30 |
| | (trifluoromethyl)phenyl}-4,5,6,7-tetrahydro-1H-pyrazolo[4,3-c]pyridin-1- | |
| | yl]-3-(4-morpholin-4-ylpiperidin-1-yl)propan-2-ol | |
| 688 | 1-(2-{[5-{1-[2-Hydroxy-3-(4-pyrrolidin-1-ylpiperidin-1-yl)propyl]-5- | 715.30 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)piperidin-4-ol | |
| 689 | (2S)-1-{3-[3-{[2-(4-Fluoropiperidin-1-yl)ethyl]sulfanyl}-4- | 716.10 |
| | (trifluoromethyl)phenyl]-4,5,6,7-tetrahydro-1H-pyrazolo[4,3-c]pyridin-1- | |
| | yl}-3-{4-[3-(trifluoromethyl)phenyl]piperazin-1-yl}propan-2-ol | |
| 690 | N-(2-{[5-{1-[2-Hydroxy-3-(4-pyridin-2-ylpiperidin-1-yl)propyl]-5- | 717.20 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)methanesulfonamide | |
| 691 | 1-[5-(Methylsulfonyl)-3-{3-[(2-morpholin-4-ylethyl)sulfanyl]-4- | 717.30 |
| | (trifluoromethyl)phenyl}-4,5,6,7-tetrahydro-1H-pyrazolo[4,3-c]pyridin-1- | |
| | yl]-3-(4-morpholin-4-ylpiperidin-1-yl)propan-2-ol | |
| 692 | 1-{3-[3-{[2-(4-Fluoropiperidin-1-yl)ethyl]sulfanyl}-4- | 717.30 |
| | (trifluoromethyl)phenyl]-5-(methylsulfonyl)-4,5,6,7-tetrahydro-1H- | |
| | pyrazolo[4,3-c]pyridin-1-yl}-3-(4-pyrrolidin-1-ylpiperidin-1-yl)propan-2-ol | |
| 693 | Ethyl (1-{2-hydroxy-3-[5-(methylsulfonyl)-3-{3-[(2-piperidin-1- | 717.30 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)carbamate | |
| 694 | N-(1-{2-Hydroxy-3-[5-(hydroxyacetyl)-3-{3-[(2-morpholin-4-yl-2- | 719.20 |
| | oxoethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)methanesulfonamide | |
| 695 | 2-Hydroxy-N-[1-(2-hydroxy-3-{3-[3-({2-[(3S)-3-hydroxypyrrolidin-1-yl]-2- | 719.24 |
| | oxoethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propyl)piperidin-4-yl]acetamide | |
| 696 | 2-Hydroxy-N-(1-{2-hydroxy-3-[5-(methylsulfonyl)-3-{3-[(2-morpholin-4-yl- | 719.24 |
| | 2-oxoethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)acetamide | |
| 697 | 2-Hydroxy-N-[1-(2-hydroxy-3-{3-[3-({2-[(3R)-3-hydroxypyrrolidin-1-yl]-2- | 719.24 |
| | oxoethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propyl)piperidin-4-yl]acetamide | |
| 698 | Ethyl (1-{2-hydroxy-3-[5-(methylsulfonyl)-3-{3-[(2-morpholin-4- | 719.30 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)carbamate | |
| 699 | 2-Hydroxy-N-[1-(2-hydroxy-3-{3-[3-{[2-(4-hydroxypiperidin-1- | 719.30 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propyl)piperidin-4-yl]acetamide | |
| 700 | N-[1-(2-Hydroxy-3-{5-(hydroxyacetyl)-3-[3-{[2-(3-hydroxypiperidin-1- | 719.30 |
| | yl)ethyl]sulfanyl}-4-(trifluoromethyl)phenyl]-4,5,6,7-tetrahydro-1H- | |
| | pyrazolo[4,3-c]pyridin-1-yl}propyl)piperidin-4-yl]methanesulfonamide | |
| 701 | (2S)-1-(1,5-Dioxa-9-azaspiro[5.5]undec-9-yl)-3-{3-[3-{[2-(4- | 720.20 |
| | fluoropiperidin-1-yl)ethyl]sulfanyl}-4-(trifluoromethyl)phenyl]-5- | |
| | (methylsulfonyl)-4,5,6,7-tetrahydro-1H-pyrazolo[4,3-c]pyridin-1- | |
| | yl}propan-2-ol | |
| 702 | 1-[4-(2-Methylphenyl)piperazin-1-yl]-3-[5-(methylsulfonyl)-3-{3-[(2- | 721.31 |
| | piperidin-1-ylethyl)sulfanyl]-4-(trifluoromethyl)phenyl}-4,5,6,7-tetrahydro- | |
| | 1H-pyrazolo[4,3-c]pyridin-1-yl]propan-2-ol | |
| 703 | 1-{4-[2-(Methyloxy)phenyl]piperidin-1-yl}-3-[5-(methylsulfonyl)-3-{3-[(2- | 722.29 |
| | pyrrolidin-1-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 704 | 1-[5-(Methylsulfonyl)-3-{3-[(2-morpholin-4-ylethyl)sulfanyl]-4- | 722.30 |
| | (trifluoromethyl)phenyl}-4,5,6,7-tetrahydro-1H-pyrazolo[4,3-c]pyridin-1- | |
| | yl]-3-[4-(phenylmethyl)piperidin-1-yl]propan-2-ol | |
| 705 | N-{1-[(2S)-3-{3-[3-{[2-(3-Fluoropiperidin-1-yl)ethyl]sulfanyl}-4- | 722.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-4-yl}-2- | |
| | hydroxyacetamide | |
| 706 | 1-(2-{[5-{1-[2-Hydroxy-3-(4-phenylpiperidin-1-yl)propyl]-5- | 722.30 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)piperidin-4-ol | |
| 707 | (2S)-1-{3-[3-{[2-(3,3-Difluoropiperidin-1-yl)ethyl]sulfanyl}-4- | 722.30 |
| | (trifluoromethyl)phenyl]-5-(methylsulfonyl)-4,5,6,7-tetrahydro-1H- | |
| | pyrazolo[4,3-c]pyridin-1-yl}-3-[4-(1,1-dimethylethyl)piperidin-1-yl]propan- | |
| | 2-ol | |
| 708 | 1-{2-Hydroxy-3-[5-(methylsulfonyl)-3-{3-[(2-piperidin-1-ylethyl)sulfanyl]- | 723.20 |
| | 4-(trifluoromethyl)phenyl}-4,5,6,7-tetrahydro-1H-pyrazolo[4,3-c]pyridin-1- | |
| | yl]propyl}-4-pyridin-2-ylpiperidin-4-ol | |
| 709 | (3S)-1-({[5-{1-[2-Hydroxy-3-(4-pyridin-2-ylpiperidin-1-yl)propyl]-5- | 723.20 |
| | (methylsulfonyl)-4,5,6,7-tetrahydro-1H-pyrazolo[4,3-c]pyridin-3-yl}-2- | |
| | (trifluoromethyl)phenyl]sulfanyl}acetyl)pyrrolidin-3-ol | |
| 710 | N-(1-{2-Hydroxy-3-[5-(methylsulfonyl)-3-{3-[(2-piperidin-1- | 723.20 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)methanesulfonamide | |
| 711 | N-[2-({5-[1-{2-Hydroxy-3-[4-(2-oxopyrrolidin-1-yl)piperidin-1-yl]propyl}-5- | 723.20 |
| | (methylsulfonyl)-4,5,6,7-tetrahydro-1H-pyrazolo[4,3-c]pyridin-3-yl]-2- | |
| | (trifluoromethyl)phenyl}sulfanyl)ethyl]methanesulfonamide | |
| 712 | 1-(2-{[5-{1-[2-Hydroxy-3-(4-phenylpiperazin-1-yl)propyl]-5- | 723.20 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)piperidin-4-ol | |
| 713 | (3R)-1-({[5-{1-[2-Hydroxy-3-(4-pyridin-2-ylpiperidin-1-yl)propyl]-5- | 723.25 |
| | (methylsulfonyl)-4,5,6,7-tetrahydro-1H-pyrazolo[4,3-c]pyridin-3-yl}-2- | |
| | (trifluoromethyl)phenyl]sulfanyl}acetyl)pyrrolidin-3-ol | |
| 714 | 1-[5-(Methylsulfonyl)-3-{3-[(2-morpholin-4-yl-2-oxoethyl)sulfanyl]-4- | 723.25 |
| | (trifluoromethyl)phenyl}-4,5,6,7-tetrahydro-1H-pyrazolo[4,3-c]pyridin-1- | |
| | yl]-3-(4-pyridin-4-ylpiperidin-1-yl)propan-2-ol | |
| 715 | N-(1-{2-Hydroxy-3-[5-(methylsulfonyl)-3-{3-[(2-pyrrolidin-1- | 723.26 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)ethanesulfonamide | |
| 716 | 1-(3-{3-[3-{[2-(Cyclopentylamino)ethyl]sulfanyl}-4- | 723.29 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)-4-pyridin-2-ylpiperidin-4-ol | |
| 717 | 1-[5-(Methylsulfonyl)-3-{3-[(2-piperidin-1-ylethyl)sulfanyl]-4- | 723.29 |
| | (trifluoromethyl)phenyl}-4,5,6,7-tetrahydro-1H-pyrazolo[4,3-c]pyridin-1- | |
| | yl]-3-[4-(pyridin-2-yloxy)piperidin-1-yl]propan-2-ol | |
| 718 | 2-(4-{2-Hydroxy-3-[5-(methylsulfonyl)-3-{3-[(2-piperidin-1- | 723.29 |
| | ylethyl)sulfanyl]-4-(trifluoromethyl)phenyl}-4,5,6,7-tetrahydro-1H- | |
| | pyrazolo[4,3-c]pyridin-1-yl]propyl}piperazin-1-yl)phenol | |
| 719 | 1-(1-{3-[3-{3-[(2-{[2-(1H-Imidazol-4-yl)ethyl]amino}ethyl)sulfanyl]-4- | 723.30 |
| | (trifluoromethyl)phenyl}-5-(methylsulfonyl)-4,5,6,7-tetrahydro-1H- | |
| | pyrazolo[4,3-c]pyridin-1-yl]propyl}piperidin-4-yl)pyrrolidin-2-one | |
| 720 | N-{1-[(2S)-3-{3-[3-{[2-(4,4-Difluoropiperidin-1-yl)ethyl]sulfanyl}-4- | 723.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-4-yl}acetamide | |
| 721 | 1-[5-(Hydroxyacetyl)-3-{3-[(2-morpholin-4-yl-2-oxoethyl)sulfanyl]-4- | 723.30 |
| | (trifluoromethyl)phenyl}-4,5,6,7-tetrahydro-1H-pyrazolo[4,3-c]pyridin-1- | |
| | yl]-3-[4-(pyrrolidin-1-ylcarbonyl)piperidin-1-yl]propan-2-ol | |
| 722 | 1-{2-Hydroxy-3-[5-(methylsulfonyl)-3-{3-[(2-morpholin-4-ylethyl)sulfanyl]- | 724.20 |
| | 4-(trifluoromethyl)phenyl}-4,5,6,7-tetrahydro-1H-pyrazolo[4,3-c]pyridin-1- | |
| | yl]propyl}-4-phenylpiperidin-4-ol | |
| 723 | (2S)-1-{3-[3-{[2-(4,4-Difluoropiperidin-1-yl)ethyl]sulfanyl}-4- | 724.30 |
| | (trifluoromethyl)phenyl]-5-(methylsulfonyl)-4,5,6,7-tetrahydro-1H- | |
| | pyrazolo[4,3-c]pyridin-1-yl}-3-[4-(1-hydroxy-1-methylethyl)piperidin-1- | |
| | yl]propan-2-ol | |
| 724 | (2S)-1-{3-[3-{[2-(3,3-Difluoropiperidin-1-yl)ethyl]sulfanyl}-4- | 724.30 |
| | (trifluoromethyl)phenyl]-5-(methylsulfonyl)-4,5,6,7-tetrahydro-1H- | |
| | pyrazolo[4,3-c]pyridin-1-yl}-3-[4-(1-hydroxy-1-methylethyl)piperidin-1- | |
| | yl]propan-2-ol | |
| 725 | 1-{2-Hydroxy-3-[5-(methylsulfonyl)-3-{3-[(2-morpholin-4-ylethyl)sulfanyl]- | 725.20 |
| | 4-(trifluoromethyl)phenyl}-4,5,6,7-tetrahydro-1H-pyrazolo[4,3-c]pyridin-1- | |
| | yl]propyl}-4-pyridin-2-ylpiperidin-4-ol | |
| 726 | 1-{3-[3-{[2-(4-Fluoropiperidin-1-yl)ethyl]sulfanyl}-4- | 725.20 |
| | (trifluoromethyl)phenyl]-5-(methylsulfonyl)-4,5,6,7-tetrahydro-1H- | |
| | pyrazolo[4,3-c]pyridin-1-yl}-3-(4-pyridin-4-ylpiperidin-1-yl)propan-2-ol | |
| 727 | N-[1-(3-{3-[3-{[2-(3,3-Difluoropyrrolidin-1-yl)ethyl]sulfanyl}-4- | 725.20 |
| | (trifluoromethyl)phenyl]-5-(hydroxyacetyl)-4,5,6,7-tetrahydro-1H- | |
| | pyrazolo[4,3-c]pyridin-1-yl}-2-hydroxypropyl)piperidin-4- | |
| | yl]methanesulfonamide | |
| 728 | 1-(2-{[5-{1-[(2S)-2-Hydroxy-3-morpholin-4-ylpropyl]-5-(methylsulfonyl)- | 725.20 |
| | 4,5,6,7-tetrahydro-1H-pyrazolo[4,3-c]pyridin-3-yl}-2- | |
| | (trifluoromethyl)phenyl]sulfanyl}ethyl)-4-pyridin-2-ylpiperidin-4-ol | |
| 729 | 1-{3-[3-{[2-(4-Fluoropiperidin-1-yl)ethyl]sulfanyl}-4- | 725.20 |
| | (trifluoromethyl)phenyl]-5-(methylsulfonyl)-4,5,6,7-tetrahydro-1H- | |
| | pyrazolo[4,3-c]pyridin-1-yl}-3-(4-phenylpiperazin-1-yl)propan-2-ol | |
| 730 | 1-(3-{3-[3-{[2-(3,3-Difluoropyrrolidin-1-yl)ethyl]sulfanyl}-4- | 725.20 |
| | (trifluoromethyl)phenyl]-5-(hydroxyacetyl)-4,5,6,7-tetrahydro-1H- | |
| | pyrazolo[4,3-c]pyridin-1-yl}-2-hydroxypropyl)-4-pyridin-2-ylpiperidin-4-ol | |
| 731 | N-(1-{2-Hydroxy-3-[5-(methylsulfonyl)-3-{3-[(2-morpholin-4- | 725.24 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)methanesulfonamide | |
| 732 | 1-[5-(Methylsulfonyl)-3-{3-[(2-morpholin-4-ylethyl)sulfanyl]-4- | 725.28 |
| | (trifluoromethyl)phenyl}-4,5,6,7-tetrahydro-1H-pyrazolo[4,3-c]pyridin-1- | |
| | yl]-3-[4-(pyridin-2-yloxy)piperidin-1-yl]propan-2-ol | |
| 733 | 1-[4-(2-Fluorophenyl)piperazin-1-yl]-3-[5-(methylsulfonyl)-3-{3-[(2- | 725.28 |
| | piperidin-1-ylethyl)sulfanyl]-4-(trifluoromethyl)phenyl}-4,5,6,7-tetrahydro- | |
| | 1H-pyrazolo[4,3-c]pyridin-1-yl]propan-2-ol | |
| 734 | (2R)-1-{3-[3-{[2-(4-Fluoropiperidin-1-yl)ethyl]sulfanyl}-4- | 725.30 |
| | (trifluoromethyl)phenyl]-5-(methylsulfonyl)-4,5,6,7-tetrahydro-1H- | |
| | pyrazolo[4,3-c]pyridin-1-yl}-3-(4-pyridin-2-ylpiperidin-1-yl)propan-2-ol | |
| 735 | (2S)-1-{3-[3-{[2-(4-Fluoropiperidin-1-yl)ethyl]sulfanyl}-4- | 725.30 |
| | (trifluoromethyl)phenyl]-5-(methylsulfonyl)-4,5,6,7-tetrahydro-1H- | |
| | pyrazolo[4,3-c]pyridin-1-yl}-3-(2-pyridin-3-ylpiperidin-1-yl)propan-2-ol | |
| 736 | 2-(4-{2-Hydroxy-3-[5-(methylsulfonyl)-3-{3-[(2-morpholin-4- | 725.30 |
| | ylethyl)sulfanyl]-4-(trifluoromethyl)phenyl}-4,5,6,7-tetrahydro-1H- | |
| | pyrazolo[4,3-c]pyridin-1-yl]propyl}piperazin-1-yl)phenol | |
| 737 | (2R)-1-{3-[3-{[2-(4-Fluoropiperidin-1-yl)ethyl]sulfanyl}-4- | 725.30 |
| | (trifluoromethyl)phenyl]-5-(methylsulfonyl)-4,5,6,7-tetrahydro-1H- | |
| | pyrazolo[4,3-c]pyridin-1-yl}-3-(4-phenylpiperazin-1-yl)propan-2-ol | |
| 738 | 1-[1-(3-{3-[3-{[2-(3,3-Dimethylpiperidin-1-yl)ethyl]sulfanyl}-4- | 725.50 |
| | (trifluoromethyl)phenyl]-5-(methylsulfonyl)-4,5,6,7-tetrahydro-1H- | |
| | pyrazolo[4,3-c]pyridin-1-yl}propyl)piperidin-4-yl]pyrrolidin-2-one | |
| 739 | 1-{3-[3-{[2-(3,3-Difluoropyrrolidin-1-yl)ethyl]sulfanyl}-4- | 726.20 |
| | (trifluoromethyl)phenyl]-5-(hydroxyacetyl)-4,5,6,7-tetrahydro-1H- | |
| | pyrazolo[4,3-c]pyridin-1-yl}-3-[4-(pyrimidin-2-yloxy)piperidin-1-yl]propan- | |
| | 2-ol | |
| 740 | 1-[5-(Methylsulfonyl)-3-{3-[(2-morpholin-4-ylethyl)sulfanyl]-4- | 726.20 |
| | (trifluoromethyl)phenyl}-4,5,6,7-tetrahydro-1H-pyrazolo[4,3-c]pyridin-1- | |
| | yl]-3-[4-(pyrimidin-2-yloxy)piperidin-1-yl]propan-2-ol | |
| 741 | (2S)-1-(4-Cyclohexylpiperidin-1-yl)-3-{3-[3-{[2-(4-methylpiperidin-1- | 726.30 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 742 | 1-[4-(2-Fluorophenyl)piperazin-1-yl]-3-[5-(methylsulfonyl)-3-{3-[(2- | 727.20 |
| | morpholin-4-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 743 | 1-[4-(3-Chloropyridin-2-yl)piperidin-1-yl]-3-[5-(methylsulfonyl)-3-{3-[(2- | 727.24 |
| | pyrrolidin-1-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 744 | N-Cyclopentyl-2-({5-[1-{2-hydroxy-3-[4-(5-oxo-4,5-dihydro-1H-1,2,4- | 727.26 |
| | triazol-3-yl)piperidin-1-yl]propyl}-5-(methylsulfonyl)-4,5,6,7-tetrahydro- | |
| | 1H-pyrazolo[4,3-c]pyridin-3-yl]-2- | |
| | (trifluoromethyl)phenyl}sulfanyl)acetamide | |
| 745 | 1-[5-(Methylsulfonyl)-3-{3-[(2-piperidin-1-ylethyl)sulfanyl]-4- | 727.32 |
| | (trifluoromethyl)phenyl}-4,5,6,7-tetrahydro-1H-pyrazolo[4,3-c]pyridin-1- | |
| | yl]-3-[4-(pyrrolidin-1-ylcarbonyl)piperidin-1-yl]propan-2-ol | |
| 746 | 1,1-Dimethylethyl (2-{3-[3-{[2-(4-fluoropiperidin-1-yl)ethyl]sulfanyl}-4- | 727.35 |
| | (trifluoromethyl)phenyl]-1-(2-hydroxy-3-piperidin-1-ylpropyl)-1,4,6,7- | |
| | tetrahydro-5H-pyrazolo[4,3-c]pyridin-5-yl}-2-oxoethyl)carbamate | |
| 747 | 1-[4-(3-Methyl-1,2,4-oxadiazol-5-yl)piperidin-1-yl]-3-[5-(methylsulfonyl)- | 728.24 |
| | 3-{3-[(2-morpholin-4-yl-2-oxoethyl)sulfanyl]-4-(trifluoromethyl)phenyl}- | |
| | 4,5,6,7-tetrahydro-1H-pyrazolo[4,3-c]pyridin-1-yl]propan-2-ol | |
| 748 | 2-(4-{2-Hydroxy-3-[5-(hydroxyacetyl)-3-{3-[(2-morpholin-4-yl-2- | 728.30 |
| | oxoethyl)sulfanyl]-4-(trifluoromethyl)phenyl}-4,5,6,7-tetrahydro-1H- | |
| | pyrazolo[4,3-c]pyridin-1-yl]propyl}piperazin-1-yl)benzonitrile | |
| 749 | 4-(2-{[5-{1-[2-Hydroxy-3-(4-pyrrolidin-1-ylpiperidin-1-yl)propyl]-5- | 728.30 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)-3-methylpiperazin-2-one | |
| 750 | 5-(1-{2-Hydroxy-3-[5-(methylsulfonyl)-3-{3-[(2-morpholin-4-yl-2- | 729.24 |
| | oxoethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)-2,4-dihydro-3H-1,2,4- | |
| | triazol-3-one | |
| 751 | 4-(1-{2-Hydroxy-3-[5-(methylsulfonyl)-3-{3-[(2-morpholin-4-yl-2- | 729.24 |
| | oxoethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)-2,4-dihydro-3H-1,2,4- | |
| | triazol-3-one | |
| 752 | 1-[5-(Methylsulfonyl)-3-{3-[(2-morpholin-4-ylethyl)sulfanyl]-4- | 729.30 |
| | (trifluoromethyl)phenyl}-4,5,6,7-tetrahydro-1H-pyrazolo[4,3-c]pyridin-1- | |
| | yl]-3-[4-(pyrrolidin-1-ylcarbonyl)piperidin-1-yl]propan-2-ol | |
| 753 | 1-[5-(Methylsulfonyl)-3-{3-[(2-pyrrolidin-1-ylethyl)sulfanyl]-4- | 729.30 |
| | (trifluoromethyl)phenyl}-4,5,6,7-tetrahydro-1H-pyrazolo[4,3-c]pyridin-1- | |
| | yl]-3-[4-(morpholin-4-ylcarbonyl)piperidin-1-yl]propan-2-ol | |
| 754 | N-Cyclopentyl-2-{[5-{1-[2-hydroxy-3-(4-morpholin-4-ylpiperidin-1- | 729.30 |
| | yl)propyl]-5-(methylsulfonyl)-4,5,6,7-tetrahydro-1H-pyrazolo[4,3- | |
| | c]pyridin-3-yl}-2-(trifluoromethyl)phenyl]sulfanyl}acetamide | |
| 755 | 1-{3-[3-({2-[3-(Hydroxymethyl)piperidin-1-yl]ethyl}sulfanyl)-4- | 729.34 |
| | (trifluoromethyl)phenyl]-5-(methylsulfonyl)-4,5,6,7-tetrahydro-1H- | |
| | pyrazolo[4,3-c]pyridin-1-yl}-3-(4-pyrrolidin-1-ylpiperidin-1-yl)propan-2-ol | |
| 756 | 1,1-Dimethylethyl 4-{[2-({5-[5-(methylsulfonyl)-1-(3-piperidin-1-ylpropyl)- | 729.34 |
| | 4,5,6,7-tetrahydro-1H-pyrazolo[4,3-c]pyridin-3-yl]-2- | |
| | (trifluoromethyl)phenyl}sulfanyl)ethyl]amino}piperidine-1-carboxylate | |
| 757 | (2S)-1-(4-Cyclohexylpiperidin-1-yl)-3-{3-[3-{[2-(3-fluoropiperidin-1- | 730.30 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 758 | 5-[1-(3-{3-[3-{[2-(4-Fluoropiperidin-1-yl)ethyl]sulfanyl}-4- | 731.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidin-4-yl]-2,4-dihydro- | |
| | 3H-1,2,4-triazol-3-one | |
| 759 | 1-{3-[3-{[2-(3,3-Difluoropyrrolidin-1-yl)ethyl]sulfanyl}-4- | 731.20 |
| | (trifluoromethyl)phenyl]-5-(methylsulfonyl)-4,5,6,7-tetrahydro-1H- | |
| | pyrazolo[4,3-c]pyridin-1-yl}-3-(4-pyrazin-2-ylpiperazin-1-yl)propan-2-ol | |
| 760 | 1-{3-[3-({2-[2-(Hydroxymethyl)morpholin-4-yl]ethyl}sulfanyl)-4- | 731.30 |
| | (trifluoromethyl)phenyl]-5-(methylsulfonyl)-4,5,6,7-tetrahydro-1H- | |
| | pyrazolo[4,3-c]pyridin-1-yl}-3-(4-pyrrolidin-1-ylpiperidin-1-yl)propan-2-ol | |
| 761 | (2S)-2-Hydroxy-N-{1-[(2S)-2-hydroxy-3-{3-[3-{[2-(4-methylpiperidin-1- | 731.32 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propyl]piperidin-4- | |
| | yl}propanamide | |
| 762 | 5-Chloro-3-{1-[2-hydroxy-3-(3-{3-[(2-piperidin-1-ylethyl)sulfanyl]-4- | 732.30 |
| | (trifluoromethyl)phenyl}-4,5,6,7-tetrahydro-1H-pyrazolo[4,3-c]pyridin-1- | |
| | yl)propyl]piperidin-4-yl}-1-methyl-1,3-dihydro-2H-benzimidazol-2-one | |
| 763 | N-[4-(Dimethylamino)phenyl]-3-[3-{[2-(4-fluoropiperidin-1- | 732.30 |
| | yl)ethyl]sulfanyl}-4-(trifluoromethyl)phenyl]-1-[(2S)-2-hydroxy-3-piperidin- | |
| | 1-ylpropyl]-1,4,6,7-tetrahydro-5H-pyrazolo[4,3-c]pyridine-5-carboxamide | |
| 764 | (4R)-1-{1-[(2S)-3-{3-[3-({2-[(3S)-3-Fluoropyrrolidin-1-yl]ethyl}sulfanyl)-4- | 733.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-4-yl}-4- | |
| | hydroxypyrrolidin-2-one | |
| 765 | 1-{2-Hydroxy-3-[5-(methylsulfonyl)-3-{3-[(2-morpholin-4-ylethyl)sulfanyl]- | 733.20 |
| | 4-(trifluoromethyl)phenyl}-4,5,6,7-tetrahydro-1H-pyrazolo[4,3-c]pyridin-1- | |
| | yl]propyl}-4-phenylpiperidine-4-carbonitrile | |
| 766 | (4S)-1-{1-[(2S)-3-{3-[3-({2-[(3S)-3-Fluoropyrrolidin-1-yl]ethyl}sulfanyl)-4- | 733.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-4-yl}-4- | |
| | hydroxypyrrolidin-2-one | |
| 767 | 1-[1-(3-{5-(Methylsulfonyl)-3-[3-({2-[(2-phenylethyl)amino]ethyl}sulfanyl)- | 733.30 |
| | 4-(trifluoromethyl)phenyl]-4,5,6,7-tetrahydro-1H-pyrazolo[4,3-c]pyridin-1- | |
| | yl}propyl)piperidin-4-yl]pyrrolidin-2-one | |
| 768 | N-[2-({5-[1-{2-Hydroxy-3-[4-(pyrimidin-2-yloxy)piperidin-1-yl]propyl}-5- | 734.20 |
| | (methylsulfonyl)-4,5,6,7-tetrahydro-1H-pyrazolo[4,3-c]pyridin-3-yl]-2- | |
| | (trifluoromethyl)phenyl}sulfanyl)ethyl]methanesulfonamide | |
| 769 | 1-[1-(3-{3-[3-{[2-(3,3-Difluoropyrrolidin-1-yl)ethyl]sulfanyl}-4- | 735.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidin-4-yl]pyrrolidin-2- | |
| | one | |
| 770 | 1-{4-[2-(Methyloxy)phenyl]piperidin-1-yl}-3-[5-(methylsulfonyl)-3-{3-[(2- | 736.31 |
| | piperidin-1-ylethyl)sulfanyl]-4-(trifluoromethyl)phenyl}-4,5,6,7-tetrahydro- | |
| | 1H-pyrazolo[4,3-c]pyridin-1-yl]propan-2-ol | |
| 771 | N-[2-({5-[1-{2-Hydroxy-3-[4-(pyrrolidin-1-ylcarbonyl)piperidin-1-yl]propyl}- | 737.20 |
| | 5-(methylsulfonyl)-4,5,6,7-tetrahydro-1H-pyrazolo[4,3-c]pyridin-3-yl]-2- | |
| | (trifluoromethyl)phenyl}sulfanyl)ethyl]methanesulfonamide | |
| 772 | N-[2-({5-[1-{2-Hydroxy-3-[4-(2-oxopyrrolidin-1-yl)piperidin-1-yl]propyl}-5- | 737.20 |
| | (methylsulfonyl)-4,5,6,7-tetrahydro-1H-pyrazolo[4,3-c]pyridin-3-yl]-2- | |
| | (trifluoromethyl)phenyl}sulfanyl)ethyl]ethanesulfonamide | |
| 773 | 1-{3-[3-({2-[3-(Hydroxymethyl)piperidin-1-yl]ethyl}sulfanyl)-4- | 737.20 |
| | (trifluoromethyl)phenyl]-5-(methylsulfonyl)-4,5,6,7-tetrahydro-1H- | |
| | pyrazolo[4,3-c]pyridin-1-yl}-3-(4-phenylpiperazin-1-yl)propan-2-ol | |
| 774 | Phenylmethyl (2-{[5-{5-acetyl-1-[2-hydroxy-3-(4-pyridin-2-ylpiperidin-1- | 737.30 |
| | yl)propyl]-4,5,6,7-tetrahydro-1H-pyrazolo[4,3-c]pyridin-3-yl}-2- | |
| | (trifluoromethyl)phenyl]sulfanyl}ethyl)carbamate | |
| 775 | 1-{3-[3-({2-[3-(Hydroxymethyl)piperidin-1-yl]ethyl}sulfanyl)-4- | 737.30 |
| | (trifluoromethyl)phenyl]-5-(methylsulfonyl)-4,5,6,7-tetrahydro-1H- | |
| | pyrazolo[4,3-c]pyridin-1-yl}-3-(4-pyridin-4-ylpiperidin-1-yl)propan-2-ol | |
| 776 | 1-{4-[2-(Methyloxy)phenyl]piperazin-1-yl}-3-[5-(methylsulfonyl)-3-{3-[(2- | 737.30 |
| | piperidin-1-ylethyl)sulfanyl]-4-(trifluoromethyl)phenyl}-4,5,6,7-tetrahydro- | |
| | 1H-pyrazolo[4,3-c]pyridin-1-yl]propan-2-ol | |
| 777 | 1-{3-[3-({2-[3-(Hydroxymethyl)piperidin-1-yl]ethyl}sulfanyl)-4- | 737.30 |
| | (trifluoromethyl)phenyl]-5-(methylsulfonyl)-4,5,6,7-tetrahydro-1H- | |
| | pyrazolo[4,3-c]pyridin-1-yl}-3-(4-pyridin-2-ylpiperidin-1-yl)propan-2-ol | |
| 778 | 1-{2-Hydroxy-3-[5-(methylsulfonyl)-3-{3-[(2-morpholin-4-yl-2- | 738.25 |
| | oxoethyl)sulfanyl]-4-(trifluoromethyl)phenyl}-4,5,6,7-tetrahydro-1H- | |
| | pyrazolo[4,3-c]pyridin-1-yl]propyl}-4-phenylpiperidin-4-ol | |
| 779 | 1-{4-[2-(Methyloxy)phenyl]piperidin-1-yl}-3-[5-(methylsulfonyl)-3-{3-[(2- | 738.30 |
| | morpholin-4-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 780 | N-{1-[(2S)-3-{3-[3-{[2-(4,4-Difluoropiperidin-1-yl)ethyl]sulfanyl}-4- | 739.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperidin-4-yl}-2- | |
| | hydroxyacetamide | |
| 781 | N-[1-(2-Hydroxy-3-{3-[3-{[2-(4-hydroxypiperidin-1-yl)ethyl]sulfanyl}-4- | 739.20 |
| | (trifluoromethyl)phenyl]-5-(methylsulfonyl)-4,5,6,7-tetrahydro-1H- | |
| | pyrazolo[4,3-c]pyridin-1-yl}propyl)piperidin-4-yl]methanesulfonamide | |
| 782 | 1-(2-Hydroxy-3-{3-[3-{[2-(4-hydroxypiperidin-1-yl)ethyl]sulfanyl}-4- | 739.20 |
| | (trifluoromethyl)phenyl]-5-(methylsulfonyl)-4,5,6,7-tetrahydro-1H- | |
| | pyrazolo[4,3-c]pyridin-1-yl}propyl)-4-pyridin-2-ylpiperidin-4-ol | |
| 783 | N-(1-{2-Hydroxy-3-[5-(methylsulfonyl)-3-{3-[(2-morpholin-4-yl-2- | 739.22 |
| | oxoethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)methanesulfonamide | |
| 784 | (3R)-1-[({5-[1-{2-Hydroxy-3-[4-(2-hydroxyphenyl)piperazin-1-yl]propyl}-5- | 739.25 |
| | (methylsulfonyl)-4,5,6,7-tetrahydro-1H-pyrazolo[4,3-c]pyridin-3-yl]-2- | |
| | (trifluoromethyl)phenyl}sulfanyl)acetyl]pyrrolidin-3-ol | |
| 785 | 1-{4-[2-(Methyloxy)phenyl]piperazin-1-yl}-3-[5-(methylsulfonyl)-3-{3-[(2- | 739.30 |
| | morpholin-4-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 786 | 1-[2-({5-[1-{2-Hydroxy-3-[4-(pyrimidin-2-yloxy)piperidin-1-yl]propyl}-5- | 740.20 |
| | (methylsulfonyl)-4,5,6,7-tetrahydro-1H-pyrazolo[4,3-c]pyridin-3-yl]-2- | |
| | (trifluoromethyl)phenyl}sulfanyl)ethyl]piperidin-4-ol | |
| 787 | 1-(3-{3-[3-{[2-(4-Fluoropiperidin-1-yl)ethyl]sulfanyl}-4- | 741.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)-4-pyridin-2-ylpiperidin-4-ol | |
| 788 | 1-[4-(2-Chlorophenyl)piperazin-1-yl]-3-[5-(methylsulfonyl)-3-{3-[(2- | 741.20 |
| | piperidin-1-ylethyl)sulfanyl]-4-(trifluoromethyl)phenyl}-4,5,6,7-tetrahydro- | |
| | 1H-pyrazolo[4,3-c]pyridin-1-yl]propan-2-ol | |
| 789 | (3R)-1-[({5-[1-{3-[4-(4-Fluorophenyl)piperazin-1-yl]-2-hydroxypropyl}-5- | 741.24 |
| | (methylsulfonyl)-4,5,6,7-tetrahydro-1H-pyrazolo[4,3-c]pyridin-3-yl]-2- | |
| | (trifluoromethyl)phenyl}sulfanyl)acetyl]pyrrolidin-3-ol | |
| 790 | 1-[4-(2-Fluorophenyl)piperazin-1-yl]-3-[5-(methylsulfonyl)-3-{3-[(2- | 741.24 |
| | morpholin-4-yl-2-oxoethyl)sulfanyl]-4-(trifluoromethyl)phenyl}-4,5,6,7- | |
| | tetrahydro-1H-pyrazolo[4,3-c]pyridin-1-yl]propan-2-ol | |
| 791 | 2-[4-(3-{3-[3-{[2-(4-Fluoropiperidin-1-yl)ethyl]sulfanyl}-4- | 741.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperazin-1-yl]phenol | |
| 792 | 1-[(2R)-3-{3-[3-{[2-(4-Fluoropiperidin-1-yl)ethyl]sulfanyl}-4- | 741.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]-4-pyridin-2-ylpiperidin-4-ol | |
| 793 | 3-{4-[(2R)-3-{3-[3-{[2-(4-Fluoropiperidin-1-yl)ethyl]sulfanyl}-4- | 741.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperazin-1-yl}phenol | |
| 794 | N-[1-(3-{3-[3-{[2-(4-Fluoropiperidin-1-yl)ethyl]sulfanyl}-4- | 741.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidin-4- | |
| | yl]methanesulfonamide | |
| 795 | 2-{4-[(2R)-3-{3-[3-{[2-(4-Fluoropiperidin-1-yl)ethyl]sulfanyl}-4- | 741.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperazin-1-yl}phenol | |
| 796 | N-{1-[(2S)-2-Hydroxy-3-{5-(methylsulfonyl)-3-[3-{[2- | 741.34 |
| | (octahydroisoquinolin-2(1H)-yl)ethyl]sulfanyl}-4-(trifluoromethyl)phenyl]- | |
| | 4,5,6,7-tetrahydro-1H-pyrazolo[4,3-c]pyridin-1-yl}propyl]piperidin-4- | |
| | yl}acetamide | |
| 797 | 1-[5-(Methylsulfonyl)-3-{3-[(2-piperidin-1-ylethyl)sulfanyl]-4- | 741.34 |
| | (trifluoromethyl)phenyl}-4,5,6,7-tetrahydro-1H-pyrazolo[4,3-c]pyridin-1- | |
| | yl]-3-[4-(piperidin-1-ylcarbonyl)piperidin-1-yl]propan-2-ol | |
| 798 | 1-{3-[3-{[2-(4-Fluoropiperidin-1-yl)ethyl]sulfanyl}-4- | 742.20 |
| | (trifluoromethyl)phenyl]-5-(methylsulfonyl)-4,5,6,7-tetrahydro-1H- | |
| | pyrazolo[4,3-c]pyridin-1-yl}-3-[4-(pyrimidin-2-yloxy)piperidin-1-yl]propan- | |
| | 2-ol | |
| 799 | 1-[4-(3-Chloropyridin-2-yl)piperazin-1-yl]-3-[5-(methylsulfonyl)-3-{3-[(2- | 742.20 |
| | piperidin-1-ylethyl)sulfanyl]-4-(trifluoromethyl)phenyl}-4,5,6,7-tetrahydro- | |
| | 1H-pyrazolo[4,3-c]pyridin-1-yl]propan-2-ol | |
| 800 | N-[2-({5-[1-{3-[4-(2-Cyanophenyl)piperazin-1-yl]-2-hydroxypropyl}-5- | 742.20 |
| | (methylsulfonyl)-4,5,6,7-tetrahydro-1H-pyrazolo[4,3-c]pyridin-3-yl]-2- | |
| | (trifluoromethyl)phenyl}sulfanyl)ethyl]methanesulfonamide | |
| 801 | 1-[5-(Methylsulfonyl)-3-{3-[(2-morpholin-4-ylethyl)sulfanyl]-4- | 742.29 |
| | (trifluoromethyl)phenyl}-4,5,6,7-tetrahydro-1H-pyrazolo[4,3-c]pyridin-1- | |
| | yl]-3-[4-(1H-pyrrolo[2,3-b]pyridin-3-yl)piperidin-1-yl]propan-2-ol | |
| 802 | 1-{3-[3-{[2-(4-Acetylpiperazin-1-yl)ethyl]sulfanyl}-4- | 742.30 |
| | (trifluoromethyl)phenyl]-5-(methylsulfonyl)-4,5,6,7-tetrahydro-1H- | |
| | pyrazolo[4,3-c]pyridin-1-yl}-3-(4-pyrrolidin-1-ylpiperidin-1-yl)propan-2-ol | |
| 803 | (2S)-1-[4-(1-Hydroxy-1-methylethyl)piperidin-1-yl]-3-{5-(methylsulfonyl)- | 742.30 |
| | 3-[3-{[2-(octahydroisoquinolin-2(1H)-yl)ethyl]sulfanyl}-4- | |
| | (trifluoromethyl)phenyl]-4,5,6,7-tetrahydro-1H-pyrazolo[4,3-c]pyridin-1- | |
| | yl}propan-2-ol | |
| 804 | 5-[1-(2-Hydroxy-3-{3-[3-({2-[3-(hydroxymethyl)piperidin-1- | 743.20 |
| | yl]ethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propyl)piperidin-4-yl]-2,4- | |
| | dihydro-3H-1,2,4-triazol-3-one | |
| 805 | (3R)-1-[({5-[1-{2-Hydroxy-3-[4-(pyrrolidin-1-ylcarbonyl)piperidin-1- | 743.28 |
| | yl]propyl}-5-(methylsulfonyl)-4,5,6,7-tetrahydro-1H-pyrazolo[4,3- | |
| | c]pyridin-3-yl]-2-(trifluoromethyl)phenyl}sulfanyl)acetyl]pyrrolidin-3-ol | |
| 806 | 1-[5-(Methylsulfonyl)-3-{3-[(2-morpholin-4-ylethyl)sulfanyl]-4- | 743.30 |
| | (trifluoromethyl)phenyl}-4,5,6,7-tetrahydro-1H-pyrazolo[4,3-c]pyridin-1- | |
| | yl]-3-[4-(piperidin-1-ylcarbonyl)piperidin-1-yl]propan-2-ol | |
| 807 | (2R)-1-[4-(2-Fluorophenyl)piperazin-1-yl]-3-{3-[3-{[2-(4-fluoropiperidin-1- | 743.30 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propan-2-ol | |
| 808 | 1-[2-({5-[1-{2-Hydroxy-3-[4-(pyrrolidin-1-ylcarbonyl)piperidin-1-yl]propyl}- | 743.30 |
| | 5-(methylsulfonyl)-4,5,6,7-tetrahydro-1H-pyrazolo[4,3-c]pyridin-3-yl]-2- | |
| | (trifluoromethyl)phenyl}sulfanyl)ethyl]piperidin-4-ol | |
| 809 | (4R)-4-Hydroxy-1-{1-[(2S)-2-hydroxy-3-{3-[3-{[2-(4-methylpiperidin-1- | 743.32 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propyl]piperidin-4-yl}pyrrolidin- | |
| | 2-one | |
| 810 | 1-[1-(2-Hydroxy-3-{3-[3-({2-[3-(hydroxymethyl)piperidin-1- | 743.32 |
| | yl]ethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propyl)piperidin-4-yl]pyrrolidin- | |
| | 2-one | |
| 811 | 1-[5-(Methylsulfonyl)-3-{3-[(2-piperidin-1-ylethyl)sulfanyl]-4- | 743.32 |
| | (trifluoromethyl)phenyl}-4,5,6,7-tetrahydro-1H-pyrazolo[4,3-c]pyridin-1- | |
| | yl]-3-[4-(morpholin-4-ylcarbonyl)piperidin-1-yl]propan-2-ol | |
| 812 | (4S)-4-Hydroxy-1-{1-[(2S)-2-hydroxy-3-{3-[3-{[2-(4-methylpiperidin-1- | 743.32 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propyl]piperidin-4-yl}pyrrolidin- | |
| | 2-one | |
| 813 | 1-[4-(3-Chloropyridin-2-yl)piperazin-1-yl]-3-[5-(methylsulfonyl)-3-{3-[(2- | 744.20 |
| | morpholin-4-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 814 | 4-Hydroxy-1-(1-{2-hydroxy-3-[5-(methylsulfonyl)-3-{3-[(2-morpholin-4-yl- | 745.26 |
| | 2-oxoethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)pyrrolidin-2-one | |
| 815 | 1-[1-(3-{3-[3-{[2-(2,3-Dihydro-1H-inden-2-ylamino)ethyl]sulfanyl}-4- | 745.30 |
| | (trifluoromethyl)phenyl]-5-(methylsulfonyl)-4,5,6,7-tetrahydro-1H- | |
| | pyrazolo[4,3-c]pyridin-1-yl}propyl)piperidin-4-yl]pyrrolidin-2-one | |
| 816 | 1-{3-[3-{[2-(4-Fluoropiperidin-1-yl)ethyl]sulfanyl}-4- | 745.30 |
| | (trifluoromethyl)phenyl]-5-(methylsulfonyl)-4,5,6,7-tetrahydro-1H- | |
| | pyrazolo[4,3-c]pyridin-1-yl}-3-[4-(pyrrolidin-1-ylcarbonyl)piperidin-1- | |
| | yl]propan-2-ol | |
| 817 | 1-[5-(Methylsulfonyl)-3-{3-[(2-morpholin-4-ylethyl)sulfanyl]-4- | 745.30 |
| | (trifluoromethyl)phenyl}-4,5,6,7-tetrahydro-1H-pyrazolo[4,3-c]pyridin-1- | |
| | yl]-3-[4-(morpholin-4-ylcarbonyl)piperidin-1-yl]propan-2-ol | |
| 818 | 2-Hydroxy-N-{1-[(2S)-2-hydroxy-3-{3-[3-{[2-(4-methylpiperidin-1- | 745.33 |
| | yl)ethyl]sulfanyl}-4-(trifluoromethyl)phenyl]-5-(methylsulfonyl)-4,5,6,7- | |
| | tetrahydro-1H-pyrazolo[4,3-c]pyridin-1-yl}propyl]piperidin-4-yl}-2- | |
| | methylpropanamide | |
| 819 | 1-{3-[3-({2-[3-(Hydroxymethyl)piperidin-1-yl]ethyl}sulfanyl)-4- | 745.33 |
| | (trifluoromethyl)phenyl]-5-(methylsulfonyl)-4,5,6,7-tetrahydro-1H- | |
| | pyrazolo[4,3-c]pyridin-1-yl}-3-(4-morpholin-4-ylpiperidin-1-yl)propan-2-ol | |
| 820 | 1-{3-[3-{[2-(3,3-Difluoropyrrolidin-1-yl)ethyl]sulfanyl}-4- | 746.20 |
| | (trifluoromethyl)phenyl]-5-(methylsulfonyl)-4,5,6,7-tetrahydro-1H- | |
| | pyrazolo[4,3-c]pyridin-1-yl}-3-[4-(pyrimidin-2-yloxy)piperidin-1-yl]propan- | |
| | 2-ol | |
| 821 | 2-(4-{2-Hydroxy-3-[5-(methylsulfonyl)-3-{3-[(2-oxo-2-piperidin-1- | 746.27 |
| | ylethyl)sulfanyl]-4-(trifluoromethyl)phenyl}-4,5,6,7-tetrahydro-1H- | |
| | pyrazolo[4,3-c]pyridin-1-yl]propyl}piperazin-1-yl)benzonitrile | |
| 822 | 4-[1-(2-Hydroxy-3-{3-[3-({2-[(3R)-3-hydroxypyrrolidin-1-yl]-2- | 747.25 |
| | oxoethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propyl)piperidin-4- | |
| | yl]benzonitrile | |
| 823 | 1-[1-(3-{3-[3-{[2-(4-Fluoropiperidin-1-yl)ethyl]sulfanyl}-4- | 747.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidin-4-yl]-4- | |
| | hydroxypyrrolidin-2-one | |
| 824 | 2-(4-{2-Hydroxy-3-[5-(methylsulfonyl)-3-{3-[(2-morpholin-4-yl-2- | 748.25 |
| | oxoethyl)sulfanyl]-4-(trifluoromethyl)phenyl}-4,5,6,7-tetrahydro-1H- | |
| | pyrazolo[4,3-c]pyridin-1-yl]propyl}piperazin-1-yl)benzonitrile | |
| 825 | 1-{3-[3-{[2-(3,3-Difluoropyrrolidin-1-yl)ethyl]sulfanyl}-4- | 749.30 |
| | (trifluoromethyl)phenyl]-5-(methylsulfonyl)-4,5,6,7-tetrahydro-1H- | |
| | pyrazolo[4,3-c]pyridin-1-yl}-3-[4-(pyrrolidin-1-ylcarbonyl)piperidin-1- | |
| | yl]propan-2-ol | |
| 826 | 1-{3-[3-{[2-(4-Acetylpiperazin-1-yl)ethyl]sulfanyl}-4- | 750.30 |
| | (trifluoromethyl)phenyl]-5-(methylsulfonyl)-4,5,6,7-tetrahydro-1H- | |
| | pyrazolo[4,3-c]pyridin-1-yl}-3-(4-phenylpiperazin-1-yl)propan-2-ol | |
| 827 | 2-{4-[(2R)-3-{3-[3-{[2-(4-Fluoropiperidin-1-yl)ethyl]sulfanyl}-4- | 750.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl]piperazin-1-yl}benzonitrile | |
| 828 | 1-{3-[3-{[2-(4-Acetylpiperazin-1-yl)ethyl]sulfanyl}-4- | 750.30 |
| | (trifluoromethyl)phenyl]-5-(methylsulfonyl)-4,5,6,7-tetrahydro-1H- | |
| | pyrazolo[4,3-c]pyridin-1-yl}-3-(4-pyridin-4-ylpiperidin-1-yl)propan-2-ol | |
| 829 | 2-[4-(3-{3-[3-{[2-(4-Fluoropiperidin-1-yl)ethyl]sulfanyl}-4- | 750.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperazin-1-yl]benzonitrile | |
| 830 | 1-{4-[2-(Ethyloxy)phenyl]piperazin-1-yl}-3-[5-(methylsulfonyl)-3-{3-[(2- | 751.32 |
| | piperidin-1-ylethyl)sulfanyl]-4-(trifluoromethyl)phenyl}-4,5,6,7-tetrahydro- | |
| | 1H-pyrazolo[4,3-c]pyridin-1-yl]propan-2-ol | |
| 831 | N′-[2-({5-[1-{2-Hydroxy-3-[4-(2-oxopyrrolidin-1-yl)piperidin-1-yl]propyl}-5- | 752.20 |
| | (methylsulfonyl)-4,5,6,7-tetrahydro-1H-pyrazolo[4,3-c]pyridin-3-yl]-2- | |
| | (trifluoromethyl)phenyl}sulfanyl)ethyl]-N,N-dimethylsulfamide | |
| 832 | N-[1-(2-Hydroxy-3-{3-[3-{[2-(2-methyl-3-oxopiperazin-1-yl)ethyl]sulfanyl}- | 752.20 |
| | 4-(trifluoromethyl)phenyl]-5-(methylsulfonyl)-4,5,6,7-tetrahydro-1H- | |
| | pyrazolo[4,3-c]pyridin-1-yl}propyl)piperidin-4-yl]methanesulfonamide | |
| 833 | 1-{4-[2-(Methyloxy)phenyl]piperidin-1-yl}-3-[5-(methylsulfonyl)-3-{3-[(2- | 752.27 |
| | morpholin-4-yl-2-oxoethyl)sulfanyl]-4-(trifluoromethyl)phenyl}-4,5,6,7- | |
| | tetrahydro-1H-pyrazolo[4,3-c]pyridin-1-yl]propan-2-ol | |
| 834 | 1-[2-({5-[1-(2-Hydroxy-3-{4-[2-(methyloxy)phenyl]piperidin-1-yl}propyl)-5- | 752.30 |
| | (methylsulfonyl)-4,5,6,7-tetrahydro-1H-pyrazolo[4,3-c]pyridin-3-yl]-2- | |
| | (trifluoromethyl)phenyl}sulfanyl)ethyl]piperidin-4-ol | |
| 835 | N-[1-(2-Hydroxy-3-{3-[3-({2-[3-(hydroxymethyl)piperidin-1- | 753.20 |
| | yl]ethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propyl)piperidin-4- | |
| | yl]methanesulfonamide | |
| 836 | 4-(2-{[5-{1-[2-Hydroxy-3-(4-hydroxy-4-pyridin-2-ylpiperidin-1-yl)propyl]-5- | 753.30 |
| | (methylsulfonyl)-4,5,6,7-tetrahydro-1H-pyrazolo[4,3-c]pyridin-3-yl}-2- | |
| | (trifluoromethyl)phenyl]sulfanyl}ethyl)-3-methylpiperazin-2-one | |
| 837 | 1,1-Dimethylethyl [1-({3-[3-{[2-(4-fluoropiperidin-1-yl)ethyl]sulfanyl}-4- | 753.30 |
| | (trifluoromethyl)phenyl]-1-(2-hydroxy-3-piperidin-1-ylpropyl)-1,4,6,7- | |
| | tetrahydro-5H-pyrazolo[4,3-c]pyridin-5- | |
| | yl}carbonyl)cyclopropyl]carbamate | |
| 838 | 2-[4-(2-Hydroxy-3-{3-[3-({2-[3-(hydroxymethyl)piperidin-1- | 753.30 |
| | yl]ethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propyl)piperazin-1-yl]phenol | |
| 839 | 1-(2-Hydroxy-3-{3-[3-({2-[3-(hydroxymethyl)piperidin-1-yl]ethyl}sulfanyl)- | 753.30 |
| | 4-(trifluoromethyl)phenyl]-5-(methylsulfonyl)-4,5,6,7-tetrahydro-1H- | |
| | pyrazolo[4,3-c]pyridin-1-yl}propyl)-4-pyridin-2-ylpiperidin-4-ol | |
| 840 | 2-[4-(3-{3-[3-{[2-(3,3-Difluoropyrrolidin-1-yl)ethyl]sulfanyl}-4- | 754.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperazin-1-yl]benzonitrile | |
| 841 | (2R)-1-{3-[3-{[2-(4-Fluoropiperidin-1-yl)ethyl]sulfanyl}-4- | 754.30 |
| | (trifluoromethyl)phenyl]-5-(methylsulfonyl)-4,5,6,7-tetrahydro-1H- | |
| | pyrazolo[4,3-c]pyridin-1-yl}-3-{4-[2-(methyloxy)phenyl]piperidin-1- | |
| | yl}propan-2-ol | |
| 842 | N-{1-[2-({5-[5-(Methylsulfonyl)-1-{3-[4-(2-oxopyrrolidin-1-yl)piperidin-1- | 754.33 |
| | yl]propyl}-4,5,6,7-tetrahydro-1H-pyrazolo[4,3-c]pyridin-3-yl]-2- | |
| | (trifluoromethyl)phenyl}sulfanyl)ethyl]piperidin-4-yl}acetamide | |
| 843 | N-[1-(2-Hydroxy-3-{3-[3-({2-[2-(hydroxymethyl)morpholin-4- | 755.20 |
| | yl]ethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propyl)piperidin-4- | |
| | yl]methanesulfonamide | |
| 844 | 1-(2-Hydroxy-3-{3-[3-({2-[2-(hydroxymethyl)morpholin-4- | 755.30 |
| | yl]ethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propyl)-4-pyridin-2-ylpiperidin- | |
| | 4-ol | |
| 845 | 1-[1-(3-{3-[3-{[2-(4-Acetylpiperazin-1-yl)ethyl]sulfanyl}-4- | 756.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidin-4-yl]pyrrolidin-2- | |
| | one | |
| 846 | N-Cyclopentyl-2-({5-[1-{2-hydroxy-3-[4-(morpholin-4- | 757.29 |
| | ylcarbonyl)piperidin-1-yl]propyl}-5-(methylsulfonyl)-4,5,6,7-tetrahydro- | |
| | 1H-pyrazolo[4,3-c]pyridin-3-yl]-2- | |
| | (trifluoromethyl)phenyl}sulfanyl)acetamide | |
| 847 | 4-[2-({5-[1-{2-Hydroxy-3-[4-(pyrrolidin-1-ylcarbonyl)piperidin-1-yl]propyl}- | 757.30 |
| | 5-(methylsulfonyl)-4,5,6,7-tetrahydro-1H-pyrazolo[4,3-c]pyridin-3-yl]-2- | |
| | (trifluoromethyl)phenyl}sulfanyl)ethyl]-3-methylpiperazin-2-one | |
| 848 | 1-[4-(3-Chloropyridin-2-yl)piperazin-1-yl]-3-[5-(methylsulfonyl)-3-{3-[(2- | 758.21 |
| | morpholin-4-yl-2-oxoethyl)sulfanyl]-4-(trifluoromethyl)phenyl}-4,5,6,7- | |
| | tetrahydro-1H-pyrazolo[4,3-c]pyridin-1-yl]propan-2-ol | |
| 849 | 1-{3-[3-{[2-(4-Acetylpiperazin-1-yl)ethyl]sulfanyl}-4- | 758.30 |
| | (trifluoromethyl)phenyl]-5-(methylsulfonyl)-4,5,6,7-tetrahydro-1H- | |
| | pyrazolo[4,3-c]pyridin-1-yl}-3-(4-morpholin-4-ylpiperidin-1-yl)propan-2-ol | |
| 850 | 1-{3-[3-({2-[2-(Hydroxymethyl)morpholin-4-yl]ethyl}sulfanyl)-4- | 759.30 |
| | (trifluoromethyl)phenyl]-5-(methylsulfonyl)-4,5,6,7-tetrahydro-1H- | |
| | pyrazolo[4,3-c]pyridin-1-yl}-3-[4-(pyrrolidin-1-ylcarbonyl)piperidin-1- | |
| | yl]propan-2-ol | |
| 851 | 1-[5-(Methylsulfonyl)-3-{3-[(2-morpholin-4-ylethyl)sulfanyl]-4- | 759.30 |
| | (trifluoromethyl)phenyl}-4,5,6,7-tetrahydro-1H-pyrazolo[4,3-c]pyridin-1- | |
| | yl]-3-{4-[(2-oxo-2-pyrrolidin-1-ylethyl)oxy]piperidin-1-yl}propan-2-ol | |
| 852 | (2R)-1-[4-(3-Chloropyridin-2-yl)piperazin-1-yl]-3-{3-[3-{[2-(4- | 760.20 |
| | fluoropiperidin-1-yl)ethyl]sulfanyl}-4-(trifluoromethyl)phenyl]-5- | |
| | (methylsulfonyl)-4,5,6,7-tetrahydro-1H-pyrazolo[4,3-c]pyridin-1- | |
| | yl}propan-2-ol | |
| 853 | 1-[1-(3-{3-[3-({2-[(1-Methyl-3-phenylpropyl)amino]ethyl}sulfanyl)-4- | 761.30 |
| | (trifluoromethyl)phenyl]-5-(methylsulfonyl)-4,5,6,7-tetrahydro-1H- | |
| | pyrazolo[4,3-c]pyridin-1-yl}propyl)piperidin-4-yl]pyrrolidin-2-one | |
| 854 | 1-{3-[3-{[2-(4-Fluoropiperidin-1-yl)ethyl]sulfanyl}-4- | 761.30 |
| | (trifluoromethyl)phenyl]-5-(methylsulfonyl)-4,5,6,7-tetrahydro-1H- | |
| | pyrazolo[4,3-c]pyridin-1-yl}-3-[4-(morpholin-4-ylcarbonyl)piperidin-1- | |
| | yl]propan-2-ol | |
| 855 | 2-[4-(2-Hydroxy-3-{3-[3-({2-[3-(hydroxymethyl)piperidin-1- | 762.30 |
| | yl]ethyl}sulfanyl)-4-(trifluoromethyl)phenyl]-5-(methylsulfonyl)-4,5,6,7- | |
| | tetrahydro-1H-pyrazolo[4,3-c]pyridin-1-yl}propyl)piperazin-1- | |
| | yl]benzonitrile | |
| 856 | 2,2,2-Trifluoro-1-{2-[3-{3-[2-(4-fluoro-piperidin-1-yl)-ethylsulfanyl]-4- | 763.30 |
| | trifluoromethyl-phenyl}-1-(2-hydroxy-3-piperidin-1-yl-propyl)-1,4,6,7- | |
| | tetrahydro-pyrazolo[4,3-c]pyridine-5-carbonyl]-pyrrolidin-1-yl}-ethanone | |
| 857 | N-[1-(3-{3-[3-{[2-(4-Acetylpiperazin-1-yl)ethyl]sulfanyl}-4- | 766.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperidin-4- | |
| | yl]methanesulfonamide | |
| 858 | 1-(3-{3-[3-{[2-(4-Acetylpiperazin-1-yl)ethyl]sulfanyl}-4- | 766.20 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)-4-pyridin-2-ylpiperidin-4-ol | |
| 859 | 2-[4-(3-{3-[3-{[2-(4-Acetylpiperazin-1-yl)ethyl]sulfanyl}-4- | 766.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperazin-1-yl]phenol | |
| 860 | 1-{3-[3-{[2-(4-Acetylpiperazin-1-yl)ethyl]sulfanyl}-4- | 770.30 |
| | (trifluoromethyl)phenyl]-5-(methylsulfonyl)-4,5,6,7-tetrahydro-1H- | |
| | pyrazolo[4,3-c]pyridin-1-yl}-3-[4-(pyrrolidin-1-ylcarbonyl)piperidin-1- | |
| | yl]propan-2-ol | |
| 861 | (2S)-2-Hydroxy-N-{1-[(2S)-2-hydroxy-3-{5-(methylsulfonyl)-3-[3-{[2- | 771.35 |
| | (octahydroisoquinolin-2(1H)-yl)ethyl]sulfanyl}-4-(trifluoromethyl)phenyl]- | |
| | 4,5,6,7-tetrahydro-1H-pyrazolo[4,3-c]pyridin-1-yl}propyl]piperidin-4- | |
| | yl}propanamide | |
| 862 | 1-{3-[3-({2-[3-(Hydroxymethyl)piperidin-1-yl]ethyl}sulfanyl)-4- | 771.35 |
| | (trifluoromethyl)phenyl]-5-(methylsulfonyl)-4,5,6,7-tetrahydro-1H- | |
| | pyrazolo[4,3-c]pyridin-1-yl}-3-[4-(piperidin-1-ylcarbonyl)piperidin-1- | |
| | yl]propan-2-ol | |
| 863 | 1-{3-[3-({2-[3-(Hydroxymethyl)piperidin-1-yl]ethyl}sulfanyl)-4- | 773.33 |
| | (trifluoromethyl)phenyl]-5-(methylsulfonyl)-4,5,6,7-tetrahydro-1H- | |
| | pyrazolo[4,3-c]pyridin-1-yl}-3-[4-(morpholin-4-ylcarbonyl)piperidin-1- | |
| | yl]propan-2-ol | |
| 864 | 1-[1-(3-{5-(Methylsulfonyl)-3-[3-{[2-(3-phenylpiperidin-1-yl)ethyl]sulfanyl}- | 773.50 |
| | 4-(trifluoromethyl)phenyl]-4,5,6,7-tetrahydro-1H-pyrazolo[4,3-c]pyridin-1- | |
| | yl}propyl)piperidin-4-yl]pyrrolidin-2-one | |
| 865 | 2-[4-(3-{3-[3-{[2-(4-Acetylpiperazin-1-yl)ethyl]sulfanyl}-4- | 775.30 |
| | (trifluoromethyl)phenyl]-5-(methylsulfonyl)-4,5,6,7-tetrahydro-1H- | |
| | pyrazolo[4,3-c]pyridin-1-yl}-2-hydroxypropyl)piperazin-1-yl]benzonitrile | |
| 866 | 1-[4-(3,5-Dichloropyridin-4-yl)piperazin-1-yl]-3-[5-(methylsulfonyl)-3-{3- | 776.21 |
| | [(2-piperidin-1-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 867 | 1,1,1-Trifluoro-N-[2-({5-[1-{2-hydroxy-3-[4-(2-oxopyrrolidin-1-yl)piperidin- | 777.20 |
| | 1-yl]propyl}-5-(methylsulfonyl)-4,5,6,7-tetrahydro-1H-pyrazolo[4,3- | |
| | c]pyridin-3-yl]-2- | |
| | (trifluoromethyl)phenyl}sulfanyl)ethyl]methanesulfonamide | |
| 868 | 1-[4-(3,5-Dichloropyridin-4-yl)piperazin-1-yl]-3-[5-(methylsulfonyl)-3-{3- | 778.10 |
| | [(2-morpholin-4-ylethyl)sulfanyl]-4-(trifluoromethyl)phenyl}-4,5,6,7- | |
| | tetrahydro-1H-pyrazolo[4,3-c]pyridin-1-yl]propan-2-ol | |
| 869 | 1,1-Dimethylethyl 4-{2-[1-(2-hydroxy-3-piperidin-1-ylpropyl)-3-{3-[(2- | 779.40 |
| | morpholin-4-ylethyl)sulfanyl]-4-(trifluoromethyl)phenyl}-1,4,6,7- | |
| | tetrahydro-5H-pyrazolo[4,3-c]pyridin-5-yl]-2-oxoethyl}piperidine-1- | |
| | carboxylate | |
| 870 | 1-[1-(3-{3-[3-{[2-(1,4′-Bipiperidin-1′-yl)ethyl]sulfanyl}-4- | 780.40 |
| | (trifluoromethyl)phenyl]-5-(methylsulfonyl)-4,5,6,7-tetrahydro-1H- | |
| | pyrazolo[4,3-c]pyridin-1-yl}propyl)piperidin-4-yl]pyrrolidin-2-one | |
| 871 | Methyl {1-[2-hydroxy-3-(4-pyridin-2-ylpiperidin-1-yl)propyl]-3-[3-{[2- | 781.30 |
| | ({[(phenylmethyl)oxy]carbonyl}amino)ethyl]sulfanyl}-4- | |
| | (trifluoromethyl)phenyl]-1,4,6,7-tetrahydro-5H-pyrazolo[4,3-c]pyridin-5- | |
| | yl}(oxo)acetate | |
| 872 | 1,1-Dimethylethyl 3-({3-[3-{[2-(4-fluoropiperidin-1-yl)ethyl]sulfanyl}-4- | 781.40 |
| | (trifluoromethyl)phenyl]-1-(2-hydroxy-3-piperidin-1-ylpropyl)-1,4,6,7- | |
| | tetrahydro-5H-pyrazolo[4,3-c]pyridin-5-yl}carbonyl)piperidine-1- | |
| | carboxylate | |
| 873 | (4S)-4-Hydroxy-1-{1-[(2S)-2-hydroxy-3-{5-(methylsulfonyl)-3-[3-{[2- | 783.35 |
| | (octahydroisoquinolin-2(1H)-yl)ethyl]sulfanyl}-4-(trifluoromethyl)phenyl]- | |
| | 4,5,6,7-tetrahydro-1H-pyrazolo[4,3-c]pyridin-1-yl}propyl]piperidin-4- | |
| | yl}pyrrolidin-2-one | |
| 874 | Ethyl {1-[2-({5-[5-(methylsulfonyl)-1-{3-[4-(2-oxopyrrolidin-1-yl)piperidin- | 784.34 |
| | 1-yl]propyl}-4,5,6,7-tetrahydro-1H-pyrazolo[4,3-c]pyridin-3-yl]-2- | |
| | (trifluoromethyl)phenyl}sulfanyl)ethyl]piperidin-4-yl}carbamate | |
| 875 | N-[2-({5-[1-{2-Hydroxy-3-[4-(2-oxopyrrolidin-1-yl)piperidin-1-yl]propyl}-5- | 785.20 |
| | (methylsulfonyl)-4,5,6,7-tetrahydro-1H-pyrazolo[4,3-c]pyridin-3-yl]-2- | |
| | (trifluoromethyl)phenyl}sulfanyl)ethyl]benzenesulfonamide | |
| 876 | 1-[1-(3-{5-(Methylsulfonyl)-3-[3-({2-[4-(phenylmethyl)piperidin-1- | 787.30 |
| | yl]ethyl}sulfanyl)-4-(trifluoromethyl)phenyl]-4,5,6,7-tetrahydro-1H- | |
| | pyrazolo[4,3-c]pyridin-1-yl}propyl)piperidin-4-yl]pyrrolidin-2-one | |
| 877 | 1-[1-(3-{5-(Methylsulfonyl)-3-[3-({2-[2-(phenylmethyl)piperidin-1- | 787.50 |
| | yl]ethyl}sulfanyl)-4-(trifluoromethyl)phenyl]-4,5,6,7-tetrahydro-1H- | |
| | pyrazolo[4,3-c]pyridin-1-yl}propyl)piperidin-4-yl]pyrrolidin-2-one | |
| 878 | 1-[1-(3-{5-(Methylsulfonyl)-3-[3-({2-[3-(phenylmethyl)piperidin-1- | 787.50 |
| | yl]ethyl}sulfanyl)-4-(trifluoromethyl)phenyl]-4,5,6,7-tetrahydro-1H- | |
| | pyrazolo[4,3-c]pyridin-1-yl}propyl)piperidin-4-yl]pyrrolidin-2-one | |
| 879 | 1-[5-(Methylsulfonyl)-3-{3-[(2-piperidin-1-ylethyl)sulfanyl]-4- | 788.33 |
| | (trifluoromethyl)phenyl}-4,5,6,7-tetrahydro-1H-pyrazolo[4,3-c]pyridin-1- | |
| | yl]-3-{4-[1-(phenylmethyl)-1H-tetrazol-5-yl]piperidin-1-yl}propan-2-ol | |
| 880 | 1-[5-(Methylsulfonyl)-3-{3-[(2-morpholin-4-ylethyl)sulfanyl]-4- | 790.30 |
| | (trifluoromethyl)phenyl}-4,5,6,7-tetrahydro-1H-pyrazolo[4,3-c]pyridin-1- | |
| | yl]-3-{4-[1-(phenylmethyl)-1H-tetrazol-5-yl]piperidin-1-yl}propan-2-ol | |
| 881 | 1-(2-Hydroxy-3-{5-(methylsulfonyl)-3-[3-{[2-(4-pyrrolidin-1-ylpiperidin-1- | 791.30 |
| | yl)ethyl]sulfanyl}-4-(trifluoromethyl)phenyl]-4,5,6,7-tetrahydro-1H- | |
| | pyrazolo[4,3-c]pyridin-1-yl}propyl)-4-phenylpiperidin-4-ol | |
| 882 | 1-(2-Hydroxy-3-{5-(methylsulfonyl)-3-[3-{[2-(4-pyrrolidin-1-ylpiperidin-1- | 792.30 |
| | yl)ethyl]sulfanyl}-4-(trifluoromethyl)phenyl]-4,5,6,7-tetrahydro-1H- | |
| | pyrazolo[4,3-c]pyridin-1-yl}propyl)-4-pyridin-2-ylpiperidin-4-ol | |
| 883 | 4-(1-{2-Hydroxy-3-[5-(methylsulfonyl)-3-{3-[(2-morpholin-4-yl-2- | 793.26 |
| | oxoethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)-2H-1,4-benzoxazin- | |
| | 3(4H)-one | |
| 884 | 1-{5-(Methylsulfonyl)-3-[3-{[2-(4-pyrrolidin-1-ylpiperidin-1- | 793.30 |
| | yl)ethyl]sulfanyl}-4-(trifluoromethyl)phenyl]-4,5,6,7-tetrahydro-1H- | |
| | pyrazolo[4,3-c]pyridin-1-yl}-3-[4-(pyrimidin-2-yloxy)piperidin-1-yl]propan- | |
| | 2-ol | |
| 885 | 2-{1-[2-Hydroxy-3-(4-pyridin-2-ylpiperidin-1-yl)propyl]-3-[3-{[2- | 795.30 |
| | ({[(phenylmethyl)oxy]carbonyl}amino)ethyl]sulfanyl}-4- | |
| | (trifluoromethyl)phenyl]-1,4,6,7-tetrahydro-5H-pyrazolo[4,3-c]pyridin-5- | |
| | yl}-2-oxoethyl acetate | |
| 886 | 1,1-Dimethylethyl 4-(2-{3-[3-{[2-(4-fluoropiperidin-1-yl)ethyl]sulfanyl}-4- | 795.42 |
| | (trifluoromethyl)phenyl]-1-(2-hydroxy-3-piperidin-1-ylpropyl)-1,4,6,7- | |
| | tetrahydro-5H-pyrazolo[4,3-c]pyridin-5-yl}-2-oxoethyl)piperidine-1- | |
| | carboxylate | |
| 887 | 5-Chloro-1-(1-{2-hydroxy-3-[5-(methylsulfonyl)-3-{3-[(2-piperidin-1- | 796.20 |
| | ylethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)-1,3-dihydro-2H- | |
| | benzimidazol-2-one | |
| 888 | Ethyl {1-[({5-[5-(methylsulfonyl)-1-{3-[4-(2-oxopyrrolidin-1-yl)piperidin-1- | 798.32 |
| | yl]propyl}-4,5,6,7-tetrahydro-1H-pyrazolo[4,3-c]pyridin-3-yl]-2- | |
| | (trifluoromethyl)phenyl}sulfanyl)acetyl]piperidin-4-yl}carbamate | |
| 889 | 5-Chloro-1-methyl-3-(1-{3-[5-(methylsulfonyl)-3-{3-[(2-morpholin-4-yl-2- | 810.24 |
| | oxoethyl)sulfanyl]-4-(trifluoromethyl)phenyl}-4,5,6,7-tetrahydro-1H- | |
| | pyrazolo[4,3-c]pyridin-1-yl]propyl}piperidin-4-yl)-1,3-dihydro-2H- | |
| | benzimidazol-2-one | |
| 890 | 1-{5-(Methylsulfonyl)-3-[3-{[2-(4-pyrrolidin-1-ylpiperidin-1- | 810.30 |
| | yl)ethyl]sulfanyl}-4-(trifluoromethyl)phenyl]-4,5,6,7-tetrahydro-1H- | |
| | pyrazolo[4,3-c]pyridin-1-yl}-3-[4-(piperidin-1-ylcarbonyl)piperidin-1- | |
| | yl]propan-2-ol | |
| 891 | 1,1-Dimethylethyl {1-[2-({5-[5-(methylsulfonyl)-1-{3-[4-(2-oxopyrrolidin-1- | 812.37 |
| | yl)piperidin-1-yl]propyl}-4,5,6,7-tetrahydro-1H-pyrazolo[4,3-c]pyridin-3- | |
| | yl]-2-(trifluoromethyl)phenyl}sulfanyl)ethyl]piperidin-4-yl}carbamate | |
| 892 | 1,1-Dimethylethyl {1-[({5-[5-(methylsulfonyl)-1-{3-[4-(2-oxopyrrolidin-1- | 826.35 |
| | yl)piperidin-1-yl]propyl}-4,5,6,7-tetrahydro-1H-pyrazolo[4,3-c]pyridin-3- | |
| | yl]-2-(trifluoromethyl)phenyl}sulfanyl)acetyl]piperidin-4-yl}carbamate | |
| 893 | 5-(Dimethylamino)-3-[1-(2-hydroxy-3-{3-[3-({2-[(3R)-3-hydroxypyrrolidin- | 836.31 |
| | 1-yl]-2-oxoethyl}sulfanyl)-4-(trifluoromethyl)phenyl]-5-(methylsulfonyl)- | |
| | 4,5,6,7-tetrahydro-1H-pyrazolo[4,3-c]pyridin-1-yl}propyl)piperidin-4-yl]-1- | |
| | methyl-1,3-dihydro-2H-imidazo[4,5-b]pyridin-2-one | |
| 894 | 5-(Dimethylamino)-3-[1-(2-hydroxy-3-{3-[3-({2-[(3S)-3-hydroxypyrrolidin- | 836.31 |
| | 1-yl]-2-oxoethyl}sulfanyl)-4-(trifluoromethyl)phenyl]-5-(methylsulfonyl)- | |
| | 4,5,6,7-tetrahydro-1H-pyrazolo[4,3-c]pyridin-1-yl}propyl)piperidin-4-yl]-1- | |
| | methyl-1,3-dihydro-2H-imidazo[4,5-b]pyridin-2-one | |
| 895 | 1,1-Dimethylethyl 4-{2-[1-(2-hydroxy-3-{4- | 836.43 |
| | [(hydroxyacetyl)amino]piperidin-1-yl}propyl)-3-{3-[(2-pyrrolidin-1- | |
| | ylethyl)sulfanyl]-4-(trifluoromethyl)phenyl}-1,4,6,7-tetrahydro-5H- | |
| | pyrazolo[4,3-c]pyridin-5-yl]-2-oxoethyl}piperidine-1-carboxylate | |
| 896 | 1-{5-(Methylsulfonyl)-3-[3-{[2-(4-pyrrolidin-1-ylpiperidin-1- | 857.40 |
| | yl)ethyl]sulfanyl}-4-(trifluoromethyl)phenyl]-4,5,6,7-tetrahydro-1H- | |
| | pyrazolo[4,3-c]pyridin-1-yl}-3-{4-[1-(phenylmethyl)-1H-tetrazol-5- | |
| | yl]piperidin-1-yl}propan-2-ol | |

### Biological Testing:

Recombinant human cathepsin S (CatS) was expressed in the baculovirus system and purified in one step with a thiopropyl-sepharose column. 10-L yielded ˜700 mg of CatS and N-terminal sequencing confirmed identity. The assay is run in 150 mM sodium acetate pH 5.0 containing 1.5 mM DTT and 150 mM NaCl. The substrate for the assay is: Z-Valine-Valine-Arginine-AMC (catalog #I-1540, Bachem). The Kₘ for the substrate is around 5 μM but the presence of substrate inhibition makes kinetic analysis difficult. With 10 μM substrate the assay rate is linear over the range of 1-8 ng CatS in 100 μL reaction. Using 2 ng/well of CatS, the production of product is linear and yields ˜7-fold signal after 20 min with only 20% loss of substrate. Measurements are taken every min for 20 min. The rate is calculated from the slope of the increase in fluorescence and the percent inhibition is calculated from this.

Results for the compounds tested in this assay are presented in Tables 2, 3, and 4 as an average of results obtained. Compounds were tested in free base, hydrochloride salt, trifluoroacetic acid salt, or citrate salt forms.

TABLE 2

| | CatS IC₅₀ |
|---|---|
| EX. | (μM) |
| 1 | 0.20 |
| 2 | 0.28 |
| 3 | 0.04 |
| 4 | 0.34 |
| 5 | 0.17 |
| 6 | 0.06 |
| 7 | 0.07 |
| 8 | 0.07 |
| 9 | 0.08 |
| 10 | 0.11 |
| 11 | 0.12 |
| 12 | 0.14 |
| 13 | 0.15 |
| 14 | 0.15 |
| 15 | 0.15 |
| 16 | 0.16 |
| 17 | 0.17 |
| 18 | 0.18 |
| 19 | 0.20 |
| 20 | 0.21 |
| 21 | 0.29 |
| 22 | 0.27 |
| 23 | 0.27 |
| 24 | 0.35 |
| 25 | 0.41 |
| 26 | 0.48 |
| 27 | 0.54 |
| 28 | 0.56 |
| 29 | 0.70 |
| 30 | 0.80 |
| 31 | 0.90 |
| 32 | 1.06 |
| 33 | 1.28 |
| 34 | 1.57 |
| 35 | 2.11 |
| 36 | 0.24 |
| 37 | 0.27 |
| 38 | 0.28 |
| 39 | 0.28 |
| 40 | 0.32 |
| 41 | 0.32 |
| 42 | 0.38 |
| 43 | 0.49 |
| 44 | 0.50 |
| 45 | 0.53 |
| 46 | 0.73 |
| 47 | 0.66 |
| 48 | 0.96 |
| 49 | 1.15 |
| 50 | 1.30 |
| 51 | 2.01 |
| 52 | 2.12 |
| 53 | 3.23 |
| 54 | 0.38 |
| 55 | 0.56 |
| 56 | 0.61 |
| 57 | 0.52 |
| 58 | 1.54 |
| 59 | 0.81 |
| 60 | 0.03 |
| 61 | 0.25 |
| 62 | 0.22 |
| 63 | 0.64 |
| 64 | NT |
| 65 | 0.10 |
| 66 | 1.10 |
| 67 | 5.55 |
| 68 | 0.50 |
| 69 | 2.90 |
| 70 | 1.85 |
| 71 | 2.25 |
| 72 | 1.05 |
| 73 | 1.03 |
| 74 | 2.00 |
| 75 | 0.09 |
| 76 | 0.10 |
| 77 | 0.12 |
| 78 | 0.14 |
| 79 | 0.18 |
| 80 | 0.18 |
| 81 | 0.19 |
| 82 | 0.20 |
| 83 | 0.28 |
| 84 | 0.42 |
| 85 | 0.52 |
| 86 | 1.15 |
| 87 | 0.58 |
| 88 | 0.66 |
| 89 | 0.49 |
| 90 | 0.22 |
| 91 | 0.15 |
| 92 | 0.06 |
| 93 | 0.08 |
| 94 | 0.07 |
| 95 | 0.07 |
| 96 | 0.42 |
| 97 | 0.13 |
| 98 | 0.19 |
| 99 | 1.26 |
| 100 | 0.23 |
| 101 | 0.36 |
| 102 | 0.37 |
| 103 | 0.77 |
| 104 | 0.33 |
| 105 | 0.28 |
| 106 | 0.29 |
| 107 | 0.28 |
| 108 | 0.09 |
| 109 | 1.23 |
| 110 | 0.40 |
| 111 | 0.24 |
| 112 | 0.32 |
| 113 | 0.06 |
| 114 | 0.38 |
| 115 | 0.13 |
| 116 | 0.19 |
| 117 | 0.20 |
| 118 | 0.36 |
| 119 | 0.39 |
| 120 | 0.53 |
| 121 | 0.28 |
| 122 | 0.33 |
| 123 | 0.23 |
| 124 | 0.23 |
| 125 | 0.42 |
| 126 | 0.58 |
| 127 | 0.85 |
| 128 | 1.65 |
| 129 | 1.47 |
| 130 | 0.74 |
| 131 | 1.25 |
| 132 | 3.60 |
| 133 | 0.60 |
| 134 | 0.13 |
| 135 | 0.35 |
| 136 | 0.68 |
| 137 | 0.22 |
| 138 | 3.10 |
| 139 | 0.63 |
| 140 | 0.08 |
| 141 | 0.05 |
| 142 | 0.04 |
| 143 | 1.20 |

TABLE 3

| | CatS IC₅₀ |
|---|---|
| EX. | (μM) |
| 144 | 3.09 |
| 145 | 1.08 |
| 146 | 0.42 |
| 147 | 0.60 |
| 148 | 1.42 |
| 149 | 0.01 |
| 150 | 0.09 |
| 151 | 0.70 |
| 152 | 0.20 |
| 153 | 0.17 |
| 154 | 0.01 |
| 155 | 0.01 |
| 156 | 0.01 |
| 157 | 0.04 |
| 158 | 0.17 |
| 159 | 0.29 |
| 160 | 0.44 |
| 161 | 0.23 |
| 162 | 0.71 |
| 163 | 1.15 |
| 164 | 0.40 |
| 165 | 0.06 |
| 166 | 0.64 |
| 167 | 0.08 |
| 168 | 0.03 |
| 169 | 0.57 |
| 170 | 0.07 |
| 171 | 0.02 |
| 172 | 0.02 |
| 173 | 0.02 |
| 174 | 0.40 |
| 175 | 0.20 |
| 176 | 0.50 |
| 177 | 0.55 |
| 178 | 0.13 |
| 179 | 0.84 |
| 180 | 0.03 |
| 181 | 0.11 |
| 182 | 0.03 |
| 183 | 0.01 |
| 184 | 0.71 |
| 185 | 0.09 |
| 186 | 1.38 |
| 187 | 1.04 |
| 188 | 0.15 |
| 189 | 0.01 |
| 190 | 0.24 |
| 191 | 0.06 |
| 192 | 0.08 |
| 193 | 0.13 |
| 194 | 0.06 |
| 195 | 0.06 |
| 196 | 0.05 |
| 197 | 0.27 |
| 198 | 0.40 |
| 199 | 0.06 |
| 200 | 0.18 |
| 201 | 0.21 |
| 202 | 0.08 |
| 203 | 0.02 |
| 204 | 0.19 |
| 205 | 0.03 |
| 206 | 0.28 |
| 207 | 0.01 |
| 208 | 0.11 |
| 209 | 0.02 |
| 210 | 0.01 |
| 211 | 0.13 |
| 212 | 0.01 |
| 213 | 0.03 |
| 214 | 0.02 |
| 215 | 1.29 |
| 216 | 0.17 |
| 217 | 0.81 |
| 218 | 0.52 |
| 219 | 0.37 |
| 220 | >20 |
| 221 | 2.50 |
| 222 | 0.96 |
| 223 | 1.10 |
| 224 | 0.38 |
| 225 | 1.52 |
| 226 | 0.92 |
| 227 | 1.13 |
| 228 | 1.99 |
| 229 | 0.13 |
| 230 | 0.18 |
| 231 | 9.52 |
| 232 | 0.16 |
| 233 | 0.17 |
| 234 | 0.02 |
| 235 | 0.04 |
| 236 | 0.33 |
| 237 | 0.13 |
| 238 | 1.08 |
| 239 | 0.28 |
| 240 | 0.04 |
| 241 | 0.03 |
| 242 | 0.58 |
| 243 | 0.60 |
| 244 | 0.02 |
| 245 | 0.80 |
| 246 | 0.19 |
| 247 | 0.09 |
| 248 | 0.26 |
| 249 | 0.05 |
| 250 | 0.05 |
| 251 | 0.23 |
| 252 | 0.59 |
| 253 | 0.47 |
| 254 | 0.99 |
| 255 | 0.10 |
| 256 | 0.002 |
| 257 | 0.03 |
| 258 | 0.01 |
| 259 | 0.06 |
| 260 | 0.07 |
| 261 | 0.01 |
| 262 | 0.04 |
| 263 | 0.14 |
| 264 | 0.08 |
| 265 | 4.40 |
| 266 | 0.10 |
| 267 | 0.41 |
| 268 | 0.11 |
| 269 | 0.63 |
| 270 | 0.39 |
| 271 | 0.06 |
| 272 | 3.58 |
| 273 | 0.19 |
| 274 | 0.01 |
| 275 | 0.01 |
| 276 | 0.01 |
| 277 | 0.02 |
| 278 | 0.10 |
| 279 | 0.39 |
| 280 | 0.19 |
| 281 | 1.56 |
| 282 | 0.26 |
| 283 | 0.28 |
| 284 | 0.07 |
| 285 | 2.65 |
| 286 | 0.04 |

TABLE 4

| | CatS IC₅₀ |
|---|---|
| EX. | (μM) |
| 287 | 1.06 |
| 288 | >20 |
| 289 | 9.57 |
| 290 | >20 |
| 291 | >20 |
| 292 | >20 |
| 293 | 2.54 |
| 294 | 4.43 |
| 295 | >20 |
| 296 | 4.13 |
| 297 | 3.85 |
| 298 | 6.02 |
| 299 | 2.11 |
| 300 | 3.15 |
| 301 | 8.43 |
| 302 | 2.41 |
| 303 | 9.03 |
| 304 | 2.86 |
| 305 | 1.56 |
| 306 | 1.26 |
| 307 | 1.13 |
| 308 | >20 |
| 309 | 3.86 |
| 310 | 13.05 |
| 311 | 3.68 |
| 312 | 0.95 |
| 313 | 1.82 |
| 314 | 2.9 |
| 315 | >20 |
| 316 | 2.54 |
| 317 | 0.16 |
| 318 | 1.44 |
| 319 | 1.31 |
| 320 | 2.26 |
| 321 | 0.39 |
| 322 | 0.23 |
| 323 | 3.18 |
| 324 | 1.83 |
| 325 | 0.36 |
| 326 | 0.88 |
| 327 | 2.48 |
| 328 | 0.83 |
| 329 | 0.17 |
| 330 | 0.29 |
| 331 | 5.93 |
| 332 | 0.81 |
| 333 | 3.11 |
| 334 | 1.03 |
| 335 | 1.39 |
| 336 | 3.88 |
| 337 | 0.69 |
| 338 | 0.81 |
| 339 | 0.17 |
| 340 | 0.36 |
| 341 | 1.4 |
| 342 | 0.44 |
| 343 | 0.66 |
| 344 | 0.28 |
| 345 | 0.26 |
| 346 | 0.37 |
| 347 | 1.85 |
| 348 | 3.82 |
| 349 | 0.52 |
| 350 | 2.32 |
| 351 | 0.78 |
| 352 | 1.26 |
| 353 | 10.06 |
| 354 | 1.27 |
| 355 | 0.21 |
| 356 | 1.14 |
| 357 | 2.7 |
| 358 | 2.11 |
| 359 | 0.14 |
| 360 | 7.54 |
| 361 | 7.68 |
| 362 | 0.1 |
| 363 | 0.16 |
| 364 | 0.26 |
| 365 | 1.9 |
| 366 | 0.55 |
| 367 | 1.05 |
| 368 | 1.34 |
| 369 | 0.27 |
| 370 | 0.28 |
| 371 | 0.25 |
| 372 | 0.38 |
| 373 | 0.47 |
| 374 | 0.83 |
| 375 | 1.36 |
| 376 | 0.15 |
| 377 | 0.29 |
| 378 | 0.56 |
| 379 | 1.29 |
| 380 | 0.31 |
| 381 | 3.85 |
| 382 | 4.76 |
| 383 | 0.8 |
| 384 | 2.23 |
| 385 | 0.35 |
| 386 | 1.19 |
| 387 | 0.37 |
| 388 | 0.58 |
| 389 | 0.22 |
| 390 | 0.28 |
| 391 | 0.29 |
| 392 | 0.57 |
| 393 | 0.59 |
| 394 | 0.31 |
| 395 | 1.43 |
| 396 | 3.96 |
| 397 | 0.54 |
| 398 | 1.55 |
| 399 | 0.41 |
| 400 | 0.68 |
| 401 | 0.5 |
| 402 | 0.51 |
| 403 | 0.63 |
| 404 | 1.1 |
| 405 | 0.77 |
| 406 | 0.55 |
| 407 | 0.24 |
| 408 | 0.33 |
| 409 | 0.59 |
| 410 | 0.7 |
| 411 | 1.82 |
| 412 | 0.49 |
| 413 | 0.39 |
| 414 | 0.48 |
| 415 | 4.97 |
| 416 | 0.18 |
| 417 | 0.56 |
| 418 | 0.12 |
| 419 | 2.53 |
| 420 | 8.08 |
| 421 | 0.26 |
| 422 | 0.16 |
| 423 | 0.2 |
| 424 | 0.61 |
| 425 | 0.1 |
| 426 | 0.27 |
| 427 | 0.095 |
| 428 | 1.14 |
| 429 | 0.82 |
| 430 | 0.23 |
| 431 | 0.23 |
| 432 | 0.27 |
| 433 | 0.68 |
| 434 | 0.54 |
| 435 | 0.065 |
| 436 | 0.075 |
| 437 | 0.62 |
| 438 | 0.57 |
| 439 | 0.58 |
| 440 | 0.74 |
| 441 | 0.31 |
| 442 | 0.2 |
| 443 | 0.06 |
| 444 | 0.18 |
| 445 | 0.28 |
| 446 | 0.21 |
| 447 | 0.2 |
| 448 | 1.4 |
| 449 | 1.11 |
| 450 | 0.94 |
| 451 | 1 |
| 452 | 1.66 |
| 453 | 0.92 |
| 454 | 0.65 |
| 455 | 1.03 |
| 456 | 0.41 |
| 457 | 0.61 |
| 458 | 0.54 |
| 459 | 2.99 |
| 460 | 2.4 |
| 461 | 0.98 |
| 462 | 2.78 |
| 463 | 6.44 |
| 464 | 0.65 |
| 465 | 0.81 |
| 466 | 0.3 |
| 467 | 0.29 |
| 468 | 0.13 |
| 469 | 0.48 |
| 470 | 0.45 |
| 471 | 0.17 |
| 472 | 0.21 |
| 473 | 0.15 |
| 474 | 0.1 |
| 475 | 4 |
| 476 | 0.55 |
| 477 | 4.53 |
| 478 | 0.35 |
| 479 | 0.48 |
| 480 | 0.13 |
| 481 | 0.24 |
| 482 | 1.1 |
| 483 | 0.41 |
| 484 | 1.58 |
| 485 | 0.25 |
| 486 | 0.29 |
| 487 | 0.33 |
| 488 | 0.32 |
| 489 | 5.1 |
| 490 | 1.03 |
| 491 | 0.55 |
| 492 | 0.43 |
| 493 | 0.34 |
| 494 | 0.2 |
| 495 | 1.67 |
| 496 | 3.56 |
| 497 | 6.15 |
| 498 | 0.4 |
| 499 | 0.53 |
| 500 | 0.25 |
| 501 | 0.15 |
| 502 | 0.37 |
| 503 | 0.17 |
| 504 | 0.22 |
| 505 | 1.82 |
| 506 | 0.88 |
| 507 | 1.07 |
| 508 | 1.88 |
| 509 | 0.66 |
| 510 | 0.32 |
| 511 | 1.19 |
| 512 | 2.65 |
| 513 | 0.17 |
| 514 | 0.23 |
| 515 | 0.19 |
| 516 | 0.36 |
| 517 | 0.54 |
| 518 | 1.36 |
| 519 | 0.8 |
| 520 | 1.49 |
| 521 | 1.13 |
| 522 | 0.98 |
| 523 | 0.49 |
| 524 | 0.2 |
| 525 | 0.91 |
| 526 | 0.2 |
| 527 | 0.57 |
| 528 | 1.54 |
| 529 | 0.15 |
| 530 | 1.2 |
| 531 | 0.19 |
| 532 | 1.25 |
| 533 | 0.095 |
| 534 | 1.06 |
| 535 | 0.94 |
| 536 | 0.2 |
| 537 | 0.15 |
| 538 | 0.22 |
| 539 | 0.16 |
| 540 | 0.18 |
| 541 | 0.2 |
| 542 | 0.61 |
| 543 | 0.34 |
| 544 | 1.39 |
| 545 | 1.32 |
| 546 | 0.65 |
| 547 | 0.23 |
| 548 | 0.21 |
| 549 | 0.57 |
| 550 | 0.22 |
| 551 | 0.7 |
| 552 | 1.58 |
| 553 | 1.92 |
| 554 | 2.12 |
| 555 | 0.24 |
| 556 | 1.25 |
| 557 | 0.3 |
| 558 | 0.35 |
| 559 | 0.25 |
| 560 | 0.29 |
| 561 | 0.21 |
| 562 | 0.22 |
| 563 | 0.15 |
| 564 | 0.23 |
| 565 | 1.16 |
| 566 | 0.14 |
| 567 | 0.41 |
| 568 | 0.55 |
| 569 | 0.57 |
| 570 | 0.64 |
| 571 | 0.47 |
| 572 | 0.34 |
| 573 | 0.46 |
| 574 | 0.13 |
| 575 | 0.19 |
| 576 | 0.085 |
| 577 | 0.35 |
| 578 | 0.31 |
| 579 | 0.12 |
| 580 | 0.56 |
| 581 | 0.55 |
| 582 | 0.53 |
| 583 | 0.78 |
| 584 | 1.24 |
| 585 | 0.23 |
| 586 | 0.49 |
| 587 | 0.64 |
| 588 | 0.18 |
| 589 | 0.16 |
| 590 | 0.33 |
| 591 | 0.2 |
| 592 | 0.19 |
| 593 | 4.89 |
| 594 | 0.2 |
| 595 | 0.41 |
| 596 | 0.59 |
| 597 | 0.11 |
| 598 | 0.53 |
| 599 | 0.01 |
| 600 | 0.03 |
| 601 | 0.53 |
| 602 | 0.9 |
| 603 | 0.08 |
| 604 | 0.13 |
| 605 | 0.22 |
| 606 | 0.35 |
| 607 | 0.59 |
| 608 | 0.18 |
| 609 | 0.34 |
| 610 | 0.27 |
| 611 | 0.34 |
| 612 | 0.09 |
| 613 | 0.32 |
| 614 | 0.19 |
| 615 | 0.36 |
| 616 | 0.025 |
| 617 | 0.24 |
| 618 | 0.13 |
| 619 | 0.25 |
| 620 | 0.85 |
| 621 | 0.025 |
| 622 | 0.13 |
| 623 | 0.14 |
| 624 | 0.29 |
| 625 | 0.09 |
| 626 | 0.23 |
| 627 | 0.5 |
| 628 | 0.19 |
| 629 | 1.16 |
| 630 | 0.27 |
| 631 | 0.29 |
| 632 | 0.16 |
| 633 | 1.18 |
| 634 | 0.51 |
| 635 | 0.93 |
| 636 | 0.41 |
| 637 | 0.095 |
| 638 | 0.32 |
| 639 | 0.085 |
| 640 | 0.63 |
| 641 | 0.22 |
| 642 | 0.27 |
| 643 | 0.06 |
| 644 | 0.63 |
| 645 | 0.045 |
| 646 | 0.36 |
| 647 | 0.31 |
| 648 | 1.28 |
| 649 | 0.092 |
| 650 | 0.43 |
| 651 | 0.095 |
| 652 | 0.07 |
| 653 | 0.4 |
| 654 | 3.14 |
| 655 | 0.08 |
| 656 | 0.09 |
| 657 | 0.17 |
| 658 | 0.33 |
| 659 | 0.035 |
| 660 | 0.025 |
| 661 | 0.11 |
| 662 | 0.023 |
| 663 | 0.035 |
| 664 | 0.065 |
| 665 | 0.065 |
| 666 | 0.48 |
| 667 | 0.085 |
| 668 | 0.12 |
| 669 | 0.17 |
| 670 | 0.12 |
| 671 | 0.12 |
| 672 | 1.34 |
| 673 | 0.14 |
| 674 | 0.18 |
| 675 | 0.89 |
| 676 | 0.02 |
| 677 | 0.09 |
| 678 | 0.07 |
| 679 | 0.24 |
| 680 | 0.11 |
| 681 | 0.08 |
| 682 | 1.03 |
| 683 | 0.27 |
| 684 | 0.17 |
| 685 | 0.18 |
| 686 | 0.17 |
| 687 | 0.11 |
| 688 | 0.58 |
| 689 | 0.39 |
| 690 | 0.01 |
| 691 | 0.27 |
| 692 | 0.28 |
| 693 | 0.065 |
| 694 | 0.085 |
| 695 | 0.085 |
| 696 | 0.035 |
| 697 | 0.065 |
| 698 | 0.14 |
| 699 | 0.13 |
| 700 | 0.15 |
| 701 | 0.2 |
| 702 | 0.02 |
| 703 | 0.03 |
| 704 | 0.15 |
| 705 | 0.065 |
| 706 | 0.015 |
| 707 | 0.07 |
| 708 | 0.055 |
| 709 | 0.008 |
| 710 | 0.12 |
| 711 | 0.045 |
| 712 | 0.055 |
| 713 | 0.004 |
| 714 | 0.02 |
| 715 | 0.68 |
| 716 | 0.06 |
| 717 | 0.06 |
| 718 | 0.015 |
| 719 | 2.34 |
| 720 | 0.035 |
| 721 | 0.045 |
| 722 | 0.15 |
| 723 | 0.045 |
| 724 | 0.075 |
| 725 | 0.17 |
| 726 | 0.03 |
| 727 | 0.05 |
| 728 | 1 |
| 729 | 0.02 |
| 730 | 0.06 |
| 731 | 0.42 |
| 732 | 0.12 |
| 733 | 0.02 |
| 734 | 0.015 |
| 735 | 7.14 |
| 736 | 0.04 |
| 737 | 0.04 |
| 738 | 0.14 |
| 739 | 0.06 |
| 740 | 0.12 |
| 741 | 0.02 |
| 742 | 0.03 |
| 743 | 0.025 |
| 744 | 1.79 |
| 745 | 0.09 |
| 746 | 0.45 |
| 747 | 0.02 |
| 748 | 0.01 |
| 749 | 0.57 |
| 750 | 0.035 |
| 751 | 0.03 |
| 752 | 0.32 |
| 753 | 0.39 |
| 754 | 0.51 |
| 755 | 0.55 |
| 756 | 2.18 |
| 757 | 0.015 |
| 758 | 0.19 |
| 759 | 0.07 |
| 760 | 0.3 |
| 761 | 0.065 |
| 762 | 0.14 |
| 763 | 0.23 |
| 764 | 0.26 |
| 765 | 0.095 |
| 766 | 0.28 |
| 767 | 0.43 |
| 768 | 0.095 |
| 769 | 0.035 |
| 770 | 0.01 |
| 771 | 0.11 |
| 772 | 0.027 |
| 773 | 0.065 |
| 774 | 0.08 |
| 775 | 0.07 |
| 776 | 0.045 |
| 777 | 0.015 |
| 778 | 0.065 |
| 779 | 0.04 |
| 780 | 0.045 |
| 781 | 0.29 |
| 782 | 0.14 |
| 783 | 0.07 |
| 784 | 0.04 |
| 785 | 0.04 |
| 786 | 0.2 |
| 787 | 0.035 |
| 788 | 0.02 |
| 789 | 0.05 |
| 790 | 0.01 |
| 791 | 0.025 |
| 792 | 0.065 |
| 793 | 0.035 |
| 794 | 0.06 |
| 795 | 0.03 |
| 796 | 0.035 |
| 797 | 0.055 |
| 798 | 0.045 |
| 799 | 0.01 |
| 800 | 0.02 |
| 801 | 0.07 |
| 802 | 1.18 |
| 803 | 0.06 |
| 804 | 0.19 |
| 805 | 0.04 |
| 806 | 0.16 |
| 807 | 0.04 |
| 808 | 0.11 |
| 809 | 0.05 |
| 810 | 0.07 |
| 811 | 0.15 |
| 812 | 0.09 |
| 813 | 0.055 |
| 814 | 0.03 |
| 815 | 0.29 |
| 816 | 0.07 |
| 817 | 0.33 |
| 818 | 0.045 |
| 819 | 0.34 |
| 820 | 0.065 |
| 821 | 0.02 |
| 822 | 0.01 |
| 823 | 0.055 |
| 824 | 0.01 |
| 825 | 0.065 |
| 826 | 0.14 |
| 827 | 0.015 |
| 828 | 0.11 |
| 829 | 0.01 |
| 830 | 0.025 |
| 831 | 0.05 |
| 832 | 0.32 |
| 833 | 0.01 |
| 834 | 0.02 |
| 835 | 0.33 |
| 836 | 0.18 |
| 837 | 0.65 |
| 838 | 0.045 |
| 839 | 0.09 |
| 840 | 0.02 |
| 841 | 0.02 |
| 842 | 1.28 |
| 843 | 0.19 |
| 844 | 0.055 |
| 845 | 0.19 |
| 846 | 0.48 |
| 847 | 0.18 |
| 848 | 0.01 |
| 849 | 0.62 |
| 850 | 0.1 |
| 851 | 0.81 |
| 852 | 0.015 |
| 853 | 1.43 |
| 854 | 0.13 |
| 855 | 0.03 |
| 856 | 1.08 |
| 857 | 0.65 |
| 858 | 0.23 |
| 859 | 0.11 |
| 860 | 0.37 |
| 861 | 0.035 |
| 862 | 0.13 |
| 863 | 0.37 |
| 864 | 0.33 |
| 865 | 0.12 |
| 866 | 0.02 |
| 867 | 0.045 |
| 868 | 0.04 |
| 869 | 1.87 |
| 870 | 2.01 |
| 871 | 0.045 |
| 872 | 1.09 |
| 873 | 0.06 |
| 874 | 0.63 |
| 875 | 0.12 |
| 876 | 0.34 |
| 877 | 0.49 |
| 878 | 0.15 |
| 879 | 0.08 |
| 880 | 0.16 |
| 881 | 0.84 |
| 882 | 0.59 |
| 883 | 0.01 |
| 884 | 0.63 |
| 885 | 0.07 |
| 886 | 0.32 |
| 887 | 0.0085 |
| 888 | 0.06 |
| 889 | 0.02 |
| 890 | 0.65 |
| 891 | 0.59 |
| 892 | 0.075 |
| 893 | 0.01 |
| 894 | 0.01 |
| 895 | 0.54 |
| 896 | 0.42 |

While the invention has been illustrated by reference to examples, it is understood that the invention is intended not to be limited to the foregoing detailed description.

What is claimed is:

1. A compound of Formula (I): 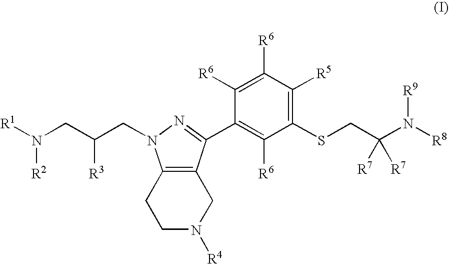
wherein:
R¹ and R² taken together with the nitrogen to which they are attached form a monocyclic heterocycloalkyl group, each group optionally containing one heteroatom ring member in addition to the nitrogen that is O, S, —N═, or NR^{a}, and each group unsubstituted or substituted with one, two, or three R^{b} substituents;
where R^{a} is H, C₁₋₄alkyl, —COC₁₋₄alkyl, or —CO₂C₁₋₄alkyl, or a phenyl ring, monocyclic cycloalkyl ring, or monocyclic heteroaryl ring, each ring unsubstituted or substituted with OH, C₁₋₄alkyl, CF₃, halo, —OC₁₋₄alkyl, cyano, or —COC₁₋₄alkyl; and
each R^{b} substituent is independently:
i) OH, C₁₋₄alkyl, CF₃, —NR^{c}R^{d}, halo, —OC₁₋₄alkyl, CH₂F, CHF₂, —C₁₋₄alkyl-OH, —CH₂NR^{c}R^{d}, —COC₁₋₄alkyl, —CO₂C₁₋₄alkyl, —CO₂H, —CONR^{m}Rⁿ, CN, or —OCH₂CONR^{m}Rⁿ;
ii) a monocyclic heterocycloalkyl group unsubstituted or substituted with C₁₋₄alkyl, OH, —OC₁₋₄alkyl, —NR^{c}R^{d}, or halo;
iii) a monocyclic heterocycloalkyl group fused with a phenyl or pyridyl group, the resulting fused bicyclic group being unsubstituted or substituted with C₁₋₄alkyl, OH, —OC₁₋₄alkyl, —NR^{c}R^{d}, or halo; or
iv) a phenyl group, benzyl group, monocyclic heteroaryl group, monocyclic cycloalkyl group, —O-phenyl group, or —O-(monocyclic heteroaryl) group, each group unsubstituted or substituted with C₁₋₄alkyl, OH, —OC₁₋₄alkyl, CF₃, cyano, —NR^{c}R^{d}, benzyl or halo; or
v) two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, —NR^{c}R^{d}, or halo; or
vi) two R^{b} substituents on adjacent carbons taken together with the carbons to which they are attached form a saturated monocyclic cycloalkyl, phenyl, or monocyclic heteroaryl group, each group unsubstituted or substituted with C₁₋₄alkyl, OH, —OC₁₋₄alkyl, —NR^{c}R^{d}, or halo;
where R^{c} and R^{d} are each independently H, C₁₋₄alkyl, —COC₁₋₄alkyl, —COC₁₋₄alkyl-OH, —CO₂C₁₋₄alkyl, —CONR^{x}R^{y}, —C₁₋₄alkyl-OH, or —SO₂C₁₋₄alkyl;
where R^{x} and R^{y} are each independently H or C₁₋₄alkyl; and
R^{m} and Rⁿ are each independently H or C₁₋₄alkyl; or R^{m} and Rⁿ taken together with the nitrogen to which they are attached form a monocyclic heterocycloalkyl ring;
R³ is H, OH, C₁₋₄alkyl, or —OC₁₋₄alkyl;
R⁴ is H; C₁₋₄alkyl; —COC₁₋₄alkyl unsubstituted or substituted with OH, F, —OCOC₁₋₄alkyl, —OC₁₋₄alkyl, phenyl, —SC₁₋₄alkyl, —NR^{o}R^{p}, or —N(R^{o})CO₂C₁₋₄alkyl; —CO-(monocyclic cycloalkyl); —CO-(monocyclic heteroaryl); —CO—(C-linked monocyclic heterocycloalkyl); —COCH₂— (monocyclic heterocycloalkyl); —SO₂C₁₋₄alkyl; —SO₂CF₃; —SO₂—(monocyclic cycloalkyl); —SO₂-phenyl; —SO₂-benzyl; —SO₂—NR^{o}R^{p}; —CONR^{o}R^{p}; —COCO₂C₁₋₄alkyl; —COCONH₂; or —COCONHC₁₋₄alkyl; wherein each cycloalkyl, heteroaryl, heterocycloalkyl, phenyl, or benzyl group is unsubstituted or substituted with C₁₋₄alkyl, OH, F, —COC₁₋₄alkyl, —COCF₃, —CO₂C₁₋₄alkyl, —N(R^{o})CO₂C₁₋₄alkyl, or —NR^{o}R^{p};
where R^{o} and R^{p} are each independently H or C₁₋₄alkyl, or a monocyclic cycloalkyl, phenyl, or monocyclic heteroaryl group; or R^{o} and R^{p} taken together with the nitrogen to which they are attached form a monocyclic heterocycloalkyl group; wherein each cycloalkyl, phenyl, heteroaryl, or heterocycloalkyl group is unsubstituted or substituted with C₁₋₄alkyl, OH, —OC₁₋₄alkyl, —NR^{s}R^{t}, or halo;
where R⁵ and R^{t} are each independently H, C₁₋₄alkyl, —COC₁₋₄alkyl, —CO₂C₁₋₄alkyl, —CONR^{v}R^{w}, C₁₋₄alkyl-OH, or —SO₂C₁₋₄alkyl;
where R^{v} and R^{w} are each independently H or C₁₋₄alkyl;
R⁵ is halo or CF₃;
each R⁶ is independently H or F;
each R⁷ is H or both R⁷ together form a carbonyl;
R⁸ is H or C₁₋₄alkyl and R⁹ is R¹⁰, —COR¹¹, —SO₂R¹¹, —SO₂CF₃, —SO₂N(C₁₋₄alkyl)₂, —CO₂C₁₋₄alkyl, or —CO₂-benzyl;
where R¹⁰ and R¹¹ are each: a C₁₋₄alkyl group unsubstituted or substituted with R^{e}; or a monocyclic cycloalkyl group, bicyclic cycloalkyl group, monocyclic heterocycloalkyl group, phenyl group, or fluorenyl group, each group unsubstituted or substituted with C₁₋₄alkyl, OH, —OC₁₋₄alkyl, —NR^{f}R^{g}, halo, —COC₁₋₄alkyl, —CO₂C₁₋₄alkyl, phenyl, benzyl, or benzyloxy;
where R^{e} is OH, —OC₁₋₄alkyl, or NR^{f}R^{g}, or a monocyclic cycloalkyl group, monocyclic heterocycloalkyl group, monocyclic heteroaryl group, phenyl group, or —O-phenyl group, each group unsubstituted or substituted with C₁₋₄alkyl, OH, —OC₁₋₄alkyl, —NR^{f}R^{g}, halo, —COC₁₋₄alkyl, or —CO₂C₁₋₄alkyl;
where R^{f} and R^{g} are each independently H, C₁₋₄alkyl, —COC₁₋₄alkyl, or —CO₂C₁₋₄alkyl;
or R⁸ and R⁹ taken together with the nitrogen to which they are attached form a monocyclic heterocycloalkyl group, each group optionally containing one heteroatom ring member in addition to the nitrogen that is O, S, SO₂, —N═, or NR^{h}, and each group unsubstituted or substituted with one or two Rⁱ substituents;
where R^{h} is H, C₁₋₄alkyl, phenyl, —C₁₋₂alkyl-phenyl, —COC₁₋₄alkyl, or —CO₂C₁₋₄alkyl; and
each Rⁱ substituent is independently: a C₁₋₄alkyl group unsubstituted or substituted with OH or phenyl; CF₃; halo; OH; —OC₁₋₄alkyl; —NR^{j}R^{k}; —COC₁₋₄alkyl; —CO₂C₁₋₄alkyl; —CONR^{j}R^{k}; phenyl; pyridyl; or benzyl; or two adjacent Rⁱ substituents taken together with the carbons to which they are attached form a phenyl or monocyclic cycloalkyl group;
where R^{j} and R^{k} are each independently H, C₁₋₄alkyl, —COC₁₋₄alkyl, —COC₁₋₄alkyl-OH, or —CO₂C₁₋₄alkyl; or R^{j} and R^{k} taken together with the nitrogen to which they are attached form a monocyclic saturated heterocycloalkyl ring;
and pharmaceutically acceptable salts, prodrugs, and metabolites thereof.

2. A compound as defined in claim 1, wherein —NR¹R² is a structure of Formula (II): 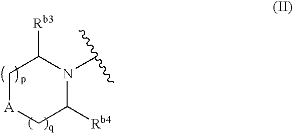
wherein:
A is NR^{a}, O, S, or C(R^{b1})(R^{b2});
where R^{a} is methyl, acetyl, or cyclopropyl; or a phenyl or pyridyl ring, each ring unsubstituted or substituted with OH, halo, —OC₁₋₄alkyl, cyano, or acetyl;
R^{b1} is H or methyl; and
R^{b2} is H, methyl, or OH; or a monocyclic heterocycloalkyl group unsubstituted or substituted with methyl, OH, or halo; or a monocyclic heterocycloalkyl group fused with a phenyl or pyridyl group, the resulting fused bicyclic group being unsubstituted or substituted with methyl, OH, dimethylamino, or halo; or a phenyl or pyridyl ring unsubstituted or substituted with methyl or halo;
one of R^{b3} and R^{b4} is H and the other is H or C₁₋₄alkyl;
p is 0, 1, or 2; and
q is 0, 1, 2, or 3;
with the proviso that when A is NR^{a}, O, S, or SO₂, then p and q are each greater than or equal to 1.

3. A compound as defined in claim 1, wherein R¹ and R² taken together with the nitrogen to which they are attached form azetidine, pyrrolidine, piperidine, piperazine substituted with R^{a}, morpholine, or thiomorpholine, each unsubstituted or substituted with one, two, or three R^{b} substituents.

4. A compound as defined in claim 1, wherein R¹ and R² taken together with the nitrogen to which they are attached form [1,4]diazepane substituted with R^{a}, azepane, 1,4,5,6-tetrahydro-pyrimidine substituted with R^{a}, 1,2,3,6-tetrahydro-pyridine, or piperazinone substituted with R^{a}, each unsubstituted or substituted with one, two, or three R^{b} substituents.

5. A compound as defined in claim 1, wherein R¹ and R² taken together with the nitrogen to which they are attached form piperidine or piperazine substituted with R^{a}, each unsubstituted or substituted with one, two, or three R^{b} substituents.

6. A compound as defined in claim 1, wherein R^{a} is H, methyl, isopropyl, acetyl, or tert-butoxycarbonyl.

7. A compound as defined in claim 1, wherein R^{a} is phenyl, cyclopropyl, pyridinyl, pyrimidinyl, or pyrazinyl ring, each optionally substituted.

8. A compound as defined in claim 1, wherein R^{a} is pyrimidin-2-yl, pyrazinyl, phenyl, 2-hydroxyphenyl, 3-hydroxyphenyl, 4-hydroxyphenyl, 2-fluorophenyl, 4-fluorophenyl, 2-cyanophenyl, 4-cyanophenyl, 2-methoxyphenyl, 3-methoxyphenyl, 4-methoxyphenyl, 2-methylphenyl, 2-ethoxyphenyl, 4-acetylphenyl, 2-chlorophenyl, 3-trifluoromethylphenyl, 3-chloropyridin-2-yl, or 3-cyano-pyridin-2-yl.

9. A compound as defined in claim 1, wherein each R^{b} substituent is independently OH, methyl, CF₃, dimethylamino, acetamido, tert-butoxycarbamoyl, fluoro, or methoxy.

10. A compound as defined in claim 1, wherein each R^{b} substituent is independently carbamoyl, tert-butyl, 1-hydroxy-1-methyl-ethyl, ureido, 2-hydroxy-acetylamino, ethoxycarbamoyl, methanesulfonylamino, pyrrolidine-1-carbonyl, amino, morpholine-4-carbonyl, dimethylaminomethyl, hydroxymethyl, acetylamino-methyl, carboxy, hydroxyethyl, difluoromethyl, piperidine-1-carbonyl, ethanesulfonylamino, 2-hydroxy-propionylamino, cyano, 2-hydroxy-2-methyl-propionylamino, or 2-oxo-2-pyrrolidin-1-yl-ethoxy.

11. A compound as defined in claim 1, wherein each R^{b} substituent is independently pyrrolidinyl, 2-oxo-pyrrolidinyl, or piperidinyl, each optionally substituted.

12. A compound as defined in claim 1, wherein each R^{b} substituent is independently pyrrolidin-1-yl, 2-oxo-pyrrolidin-1-yl, 5-dimethylamino-1-methyl-1,3-dihydro-imidazo[4,5-b]pyridin-2-onyl, or 4-4H-benzo[1,4]oxazin-3-onyl.

13. A compound as defined in claim 1, wherein each R^{b} substituent is independently 5-oxo-4,5-dihydro-1H-[1,2,4]triazol-3-yl, morpholin-1-yl, pyrrolidin-2-on-1-yl, 4-hydroxy-2-oxo-pyrrolidin-1-yl, piperidin-1-yl, 5-oxo-1,5-dihydro-[1,2,4]triazol-4-yl, 5-chloro-1-methyl-1,3-dihydro-2H-benzimidazol-2-on-1-yl, or 5-chloro-1,3-dihydro-2H-benzimidazol-2-on-1-yl.

14. A compound as defined in claim 1, wherein each R^{b} substituent is independently phenyl, pyridyl, pyrimidinyl, oxadiazolyl, 1H-pyrrolo[2,3-b]pyridin-3-yl, tetrazolyl, pyrimidinyloxy, pyridinyloxy, cyclohexyl, or benzyl, each optionally substituted.

15. A compound as defined in claim 1, wherein each R^{b} substituent is independently phenyl, 4-cyanophenyl, 3-cyanophenyl, 2-methoxyphenyl, 4-methoxyphenyl, 3-chlorophenyl, 3-trifluoromethylphenyl, 2-pyridinyl, 3-pyridinyl, 4-pyridinyl, 2-pyrimidinyl, 3-methyl-[1,2,4]oxadiazol-5-yl, 3-chloro-pyridin-2-yl, 1H-pyrrolo[2,3-b]pyridin-3-yl, 3,5-dichloro-pyridin-4-yl, 1-benzyl-1H-tetrazol-5-yl, cyclohexyl, benzyl, hydroxytriazolyl, pyrimidin-2-yloxy, or pyridin-2-yloxy.

16. A compound as defined in claim 1, wherein two R^{b} substituents at the same carbon taken together with the carbon to which they are attached form a [1,3]dioxolane or [1,3]dioxane ring, each optionally substituted.

17. A compound as defined in claim 1, wherein two R^{b} substituents on adjacent carbons taken together with the monocyclic heterocycloalkyl group to which they are attached form a fused ring system selected from the group consisting of hexahydro-cyclopenta[c]pyrrol-2-yl, 1,3-dihydro-isoindol-2-yl, 2,4,5,7-tetrahydro-pyrazolo[3,4-c]pyridin-6-yl, 1,4,6,7-tetrahydro-imidazo[4,5-c]pyridin-5-yl, 3,4-dihydro-1H-isoquinolin-2-yl, or octahydro-isoquinolin-2-yl, each optionally substituted.

18. A compound as defined in claim 1, wherein R³ is H or OH.

19. A compound as defined in claim 1, wherein R⁴ is H, methyl, ethyl, acetyl, 2-hydroxyacetyl, 2-methoxyacetyl, 2-acetoxyacetyl, propionyl, 2-hydroxy-propionyl, 2-hydroxy-2-methyl-propionyl, 2-hydroxy-2-phenyl-acetyl, 2-aminoacetyl, 2-dimethylamino-acetyl, 2-tert-butoxycarbonylamino-acetyl, 2-methylsulfanyl-acetyl, 2-(piperidin-4-yl)-acetyl, 2-(1-tert-butoxycarbonyl-piperidin-4-yl)-acetyl, or methoxyoxalyl.

20. A compound as defined in claim 1, wherein R⁴ is aminooxalyl, methylaminooxalyl, carbamoyl, methylcarbamoyl, dimethylcarbamoyl, isopropylcarbamoyl, sec-butylcarbamoyl, tert-butylcarbamoyl, diethylcarbamoyl, diisopropylcarbamoyl, pyrrolidine-1-carbonyl, morpholine-4-carbonyl, phenylcarbamoyl, cyclohexylcarbamoyl, thiophen-2-ylcarbamoyl, 4-methyl-piperazine-1-carbonyl, or (4-dimethylaminophenyl)carbamoyl.

21. A compound as defined in claim 1, wherein R⁴ is ethanesulfonyl, propanesulfonyl, isopropanesulfonyl, trifluoromethanesulfonyl, cyclopropanesulfonyl, benzenesulfonyl, benzylsulfonyl, or dimethylsulfamoyl.

22. A compound as defined in claim 1, wherein R⁴ is cyclobutanecarbonyl, 1-amino-cyclopropanecarbonyl, 1-tert-butoxycarbonylamino-cyclopropanecarbonyl, 1H-[1,2,4]triazole-3-carbonyl, 1-methyl-1H-imidazole-2-carbonyl, thiophene-2-carbonyl, furan-2-carbonyl, tetrahydrofuran-2-carbonyl, 1-methyl-pyrrolidine-2-carbonyl, 1-acetyl-pyrrolidine-2-carbonyl, 1-trifluoroacetyl-pyrrolidine-2-carbonyl, piperidine-3-carbonyl, or 1-tert-butoxycarbonyl-piperidine-3-carbonyl.

23. A compound as defined in claim 1, wherein R⁴ is —SO₂CH₃, —CONH₂, or —COCONH₂.

24. A compound as defined in claim 1, wherein R⁴ is —SO₂CH₃.

25. A compound as defined in claim 1, wherein R⁵ is chloro or CF₃.

26. A compound as defined in claim 1, wherein R⁵ is CF₃.

27. A compound as defined in claim 1, wherein each R⁶ is H.

28. A compound as defined in claim 1, wherein each R⁷ is H.

29. A compound as defined in claim 1, wherein both R⁷ together form a carbonyl.

30. A compound as defined in claim 1, wherein R⁸ is H or methyl.

31. A compound as defined in claim 1, wherein R⁹ is a methyl, ethyl, propyl, isopropyl, sec-butyl, iso-butyl, or tert-butyl group, each unsubstituted or substituted with R^{e}.

32. A compound as defined in claim 1, wherein R⁹ is R¹⁰, and R¹⁰ is selected from the group consisting of cyclopropyl, cyclobutyl, cyclopentyl, cyclohexyl, indanyl, and piperidin-4-yl, each unsubstituted or substituted with methyl, OH, methoxy, dimethylamino, tert-butoxycarbonyl, or halo.

33. A compound as defined in claim 1, wherein R¹⁰ is a phenyl group, unsubstituted or substituted with methyl, OH, halo, dimethylamino, phenyl, or benzyloxy.

34. A compound as defined in claim 1, wherein R¹⁰ is a benzyl or phenethyl group.

35. A compound as defined in claim 1, wherein R⁹ is —COR¹¹, and R¹¹ is a methyl, ethyl, or propyl group, each unsubstituted or substituted with R^{e}.

36. A compound as defined in claim 1, wherein R¹¹ is selected from the group consisting of cyclopropyl, cyclobutyl, cyclopentyl, cyclohexyl, indanyl, and piperidin-4-yl, each unsubstituted or substituted with methyl, OH, methoxy, or halo.

37. A compound as defined in claim 1, wherein R¹¹ is a phenyl group, unsubstituted or substituted with methyl, ethyl, OH, halo, dimethylamino, or tert-butoxycarbonyl.

38. A compound as defined in claim 1, wherein R^{e} is OH, methoxy, dimethylamino, diethylamino, or tert-butoxycarbamoyl.

39. A compound as defined in claim 1, wherein R^{e} is a cyclopropyl, cyclopentyl, cyclohexyl, furanyl, thiophenyl, pyridyl, phenyl, phenoxy, or imidazolyl group, each unsubstituted or substituted with methyl, OH, methoxy, dimethylamino, or halo.

40. A compound as defined in claim 1, wherein R^{e} is OH, methoxy, thiophen-2-yl, cyclohexenyl, phenoxy, piperidinyl, pyrrolidinyl, or tert-butoxycarbamoyl.

41. A compound as defined in claim 1, wherein R⁹ is tert-butoxycarbonyl, cyclopentyl, cyclohexyl, benzyl, methyl, 3-methoxypropyl, thiophen-2-ylmethyl, 2-cyclohex-1-enyl-ethyl, 2-phenoxy-ethyl, 2-morpholin-4-yl-ethyl, 1-ethyl-pyrrolidin-2-ylmethyl, 4-biphenyl, indan-2-yl, 3-biphenyl, 9H-fluoren-1-yl, 4-benzyloxy-phenyl, phenethyl, 4-fluorophenyl, or 1-methyl-piperidin-4-yl.

42. A compound as defined in claim 1, wherein R⁹ is propyl, isopropyl, isobutyl, cyclopropyl, 1-methyl-3-phenyl-propyl, 3-hydroxy-propyl, 2-(1H-imidazol-4-yl)-ethyl, or 1-(tert-butoxycarbonyl)-piperidin-4-yl.

43. A compound as defined in claim 1, wherein R⁹ is benzoyl, phenylacetyl, 1-tert-butoxycarbonyl-piperidine-4-carbonyl, tert-butylcarbamoyl-acetyl, 4-fluorobenzoyl, 4-hydroxybenzoyl, 4-dimethylamino-benzoyl, 4-fluorophenylacetyl, 1-tert-butoxycarbonyl-piperidine-3-carbonyl, 1-tert-butoxycarbonyl-pyrrolidine-3-carbonyl, 4-tert-butylcarbamoyl-1-butyryl, piperidine-3-carbonyl, pyrrolidine-3-carbonyl, piperidine-4-carbonyl, or benzyloxycarbonyl.

44. A compound as defined in claim 1, wherein R⁹ is —SO₂R¹¹, —SO₂CF₃, or —SO₂N(C₁₋₄alkyl)₂.

45. A compound as defined in claim 1, wherein R⁹ is methanesulfonyl, ethanesulfonyl, dimethylsulfamoyl, trifluoromethanesulfonyl, or benzenesulfonyl.

46. A compound as defined in claim 1, wherein R⁸ and R⁹ taken together with the nitrogen to which they are attached form azetidine, pyrrolidine, piperidine, piperazine substituted with R^{h}, morpholine, thiomorpholine, 1,1-dioxo-1λ⁶-thiomorpholine, azepine, diazepine substituted with R^{h}, 3,6-dihydro-2H-pyridine, or thiazolidine, each unsubstituted or substituted with Rⁱ.

47. A compound as defined in claim 1, wherein R⁸ and R⁹ taken together with the nitrogen to which they are attached form pyrrolidine or piperidine, each unsubstituted or substituted with R^{h} and Rⁱ.

48. A compound as defined in claim 1, wherein R⁸ and R⁹ taken together with the nitrogen to which they are attached form piperazine, morpholine, azetidine, oxo-imidazolidin-1-yl, dihydro-pyrimidin-1-yl, or oxo-piperazinyl.

49. A compound as defined in claim 1, wherein R^{h} is H, methyl, isopropyl, acetyl, tert-butoxycarbonyl, phenyl, or phenethyl.

50. A compound as defined in claim 1, wherein each Rⁱ substituent is independently: a methyl or ethyl group unsubstituted or substituted with OH or phenyl; tert-butyl; CF₃; fluoro; OH; methoxy; dimethylamino; acetylamino; 2-hydroxy-acetylamino; ethoxycarbonylamino; tert-butoxycarbonylamino; piperidin-1-yl; pyrrolidin-1-yl; acetyl; methoxycarbonyl; carbamoyl; dimethylcarbamoyl; diethylcarbamoyl; phenyl; pyridyl; or benzyl; or two adjacent Rⁱ substituents taken together with the carbons to which they are attached form phenyl, cyclopentyl, or cyclohexyl.

51. A compound as defined in claim 1, wherein R⁸ and R⁹ taken together with the nitrogen to which they are attached form piperidin-1-yl, 4-hydroxy-piperidin-1-yl, octahydro-isoquinolin-2-yl, 4,4-difluoro-piperidin-1-yl, 3-methyl-piperidin-1-yl, 3,5-dimethyl-piperidin-1-yl, 4-methyl-piperidin-1-yl, 3,3-difluoro-piperidin-1-yl, octahydro-quinolin-1-yl, 3-hydroxymethyl-piperidin-1-yl, 3,6-dihydro-2H-pyridin-1-yl, 1,1-dioxo-1λ⁶-thiomorpholin-4-yl, 4-tert-butyl-piperidin-1-yl, 3-fluoro-piperidin-1-yl, 2-methyl-piperidin-1-yl, 4-phenyl-piperidin-1-yl, 2-trifluoromethyl-pyrrolidin-1-yl, morpholin-1-yl, 4-(2-hydroxy-ethyl)-piperidin-1-yl, pyrrolidin-1-yl, 4-phenyl-piperazin-1-yl, 2-methyl-pyrrolidin-1-yl, 4-methyl-piperazin-1-yl, 4-dimethylamino-piperidin-1-yl, 2-phenyl-piperidin-1-yl, 4-carbamoyl-piperidin-1-yl, 4-methoxy-piperidin-1-yl, 3-trifluoromethyl-piperidin-1-yl, 2-hydroxymethyl-piperidin-1-yl, 4-trifluoromethyl-piperidin-1-yl, 2-thiazolidin-3-yl, 3-hydroxy-pyrrolidin-1-yl, 3,4-dihydro-1H-isoquinolin-2-yl, 4-methyl-[1,4]diazepan-1-yl, 3-diethylcarbamoyl-piperidin-1-yl, 3-dimethylamino-pyrrolidin-1-yl, 4-phenyl-3,6-dihydro-2H-pyridin-1-yl, [1,4′]bipiperidinyl-1′-yl, 2-azepan-1-yl, 4-benzyl-piperidin-1-yl, 4-phenethyl-piperidin-1-yl, 4-diethylcarbamoyl-piperidin-1-yl, 2-carbamoyl-pyrrolidin-1-yl, 4-fluoro-piperidin-1-yl, 4-isopropyl-piperazin-1-yl, or 4-phenethyl-piperazin-1-yl.

52. A compound as defined in claim 1, wherein R⁸ and R⁹ taken together with the nitrogen to which they are attached form 3-hydroxy-piperidin-1-yl, 4-(acetylamino)-piperidin-1-yl, 3-hydroxymethyl-piperidin-1-yl, 4-(pyrrolidin-1-yl)-piperidin-1-yl, 4-(1-hydroxy-1-methyl-ethyl)-piperidin-1-yl, 4-(2-hydroxy-acetylamino)-piperidin-1-yl, 4-hydroxy-4-(2-pyridinyl)-piperidin-1-yl, 3,3-dimethyl-piperidin-1-yl, 3-phenyl-piperidin-1-yl, 4-ethoxycarbonylamino-piperidin-1-yl, 2-benzyl-piperidin-1-yl, 3-benzyl-piperidin-1-yl, 4-tert-butoxycarbonylamino-piperidin-1-yl, 4-acetyl-piperazin-1-yl, 3-methyl-morpholin-4-yl, 2,6-dimethyl-morpholin-4-yl, 3-hydroxymethyl-morpholin-4-yl, 2-hydroxylmethyl-morpholin-4-yl, 3,3-difluoro-pyrrolidin-1-yl, 3-fluoro-pyrrolidin-1-yl, 3-hydroxymethyl-pyrrolidin-1-yl, azetidinyl, 4-oxo-imidazolidin-1-yl, 5,6-dihydro-4H-pyrimidin-1-yl, 2-methyl-3-oxo-piperazin-1-yl), or hexahydro-cyclopenta[c]pyrrol-2-yl.

53. A compound as defined in claim 1, wherein compounds of Formula (I) are selected from compounds of the following Formula (IV): 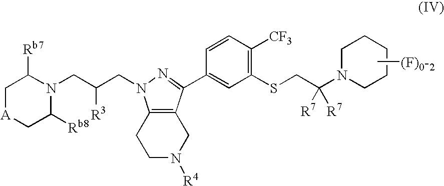
A is NR^{a}, O, S, or C(R^{b5})(R^{b6});
where R^{a} is methyl, acetyl, or cyclopropyl; or a phenyl or pyridyl ring, each ring unsubstituted or substituted with OH, halo, —OC₁₋₄alkyl, cyano, or acetyl;
R^{b5} is H or methyl; and
R^{b6} is H, methyl, or OH; or a monocyclic heterocycloalkyl group unsubstituted or substituted with methyl, OH, or halo; or a monocyclic heterocycloalkyl group fused with a phenyl or pyridyl group, the resulting fused bicyclic group being unsubstituted or substituted with methyl, OH, dimethylamino, or halo; or a phenyl or pyridyl ring unsubstituted or substituted with methyl or halo;
one of R^{b7} and R^{b8} is H and the other is H or C₁₋₄alkyl;
R³ is H or OH;
R⁴ is —SO₂CH₃, —CONH₂, or —COCONH₂; and
each R⁷ is H or both R⁷ together form a carbonyl;
and pharmaceutically acceptable salts, prodrugs, and metabolites thereof.

54. A compound selected from the group consisting of:
1-{1-[3-(3-{3-[2-(4-Hydroxy-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(octahydro-isoquinolin-2-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4,4-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(3-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3,5-Dimethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(octahydro-quinolin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,6-Dihydro-2H-pyridin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(1,1-Dioxo-1λ⁶-thiomorpholin-4-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(4-tert-Butyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Fluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
(S)-1-{1-[3-(5-Methanesulfonyl-3-{4-trifluoromethyl-3-[2-(2-trifluoromethyl-pyrrolidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{5-Methanesulfonyl-3-[3-(2-morpholin-4-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{3-[3-(2-Cyclopentylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-(1-{3-[3-(3-{2-[4-(2-Hydroxy-ethyl)-piperidin-1-yl]-ethylsulfanyl}-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one;
1-[1-(3-{5-Methanesulfonyl-3-[3-(2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-phenyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-[3-[2-(Cyclohexyl-methyl-amino)-ethylsulfanyl]-4-trifluoromethyl-phenyl]-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-pyrrolidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-[1-(3-{3-[3-(2-Benzylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-methylamino-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(3-methoxy-propylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-[1-(3-{3-[3-(2-Dimethylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{3-[2-(4-Dimethylamino-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl}-piperidine-4-carboxylic acid amide;
1-{1-[3-(5-Methanesulfonyl-3-{3-[2-(4-methoxy-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{4-trifluoromethyl-3-[2-(3-trifluoromethyl-piperidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(2-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{4-trifluoromethyl-3-[2-(4-trifluoromethyl-piperidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-[1-(3-{5-Methanesulfonyl-3-[3-(2-thiazolidin-3-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-(1-{3-[5-Methanesulfonyl-3-(3-{2-[(thiophen-2-ylmethyl)-amino]-ethylsulfanyl}-4-trifluoromethyl-phenyl)-4,5,6,7-tetrahydro-pyrazolo[4,3-c]pyridin-1-yl]-propyl}-piperidin-4-yl)-pyrrolidin-2-one;
1-{1-[3-(3-{3-[2-(3-Hydroxy-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,4-Dihydro-1H-isoquinolin-2-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-[1,4]diazepan-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(2-Cyclohex-1-enyl-ethylamino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-phenoxy-ethylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-morpholin-4-yl-ethylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-phenethyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-(1-{3-[3-(3-{2-[(1-Ethyl-pyrrolidin-2-ylmethyl)-amino]-ethylsulfanyl}-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one;
1-{1-[3-(3-{3-[2-(Benzyl-methyl-amino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl}-piperidine-3-carboxylic acid diethylamide;
1-{1-[3-(3-{3-[2-(3-Dimethylamino-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
5-Methanesulfonyl-3-[3-(2-piperidin-1-yl-ethylsulfanyl)-4-trifluoromethyl-phenyl]-1-(3-piperidin-1-yl-propyl)-4,5,6,7-tetrahydro-1H-pyrazolo[4,3-c]pyridine;
5-Methanesulfonyl-3-[3-(2-piperidin-1-yl-ethylsulfanyl)-4-trifluoromethyl-phenyl]-1-[3-(4-pyrrolidin-1-yl-piperidin-1-yl)-propyl]-4,5,6,7-tetrahydro-1H-pyrazolo[4,3-c]pyridine;
1-[4-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperazin-1-yl]-ethanone;
5-Methanesulfonyl-3-[3-(2-piperidin-1-yl-ethylsulfanyl)-4-trifluoromethyl-phenyl]-1-(3-pyrrolidin-1-yl-propyl)-4,5,6,7-tetrahydro-1H-pyrazolo[4,3-c]pyridine;
1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-pyrrolidin-3-ol;
5-Methanesulfonyl-1-(3-morpholin-4-yl-propyl)-3-[3-(2-piperidin-1-yl-ethylsulfanyl)-4-trifluoromethyl-phenyl]-4,5,6,7-tetrahydro-1H-pyrazolo[4,3-c]pyridine;
4-[1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-4H-benzo[1,4]oxazin-3-one;
{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-carbamic acid tert-butyl ester;
1-[1-(2-Hydroxy-3-{5-methanesulfonyl-3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
N-Biphenyl-4-yl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-(2-phenoxy-ethyl)-acetamide;
1-{1-[2-Hydroxy-3-(5-methanesulfonyl-3-{3-[2-oxo-2-(4-phenyl-3,6-dihydro-2H-pyridin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-indan-2-yl-acetamide;
N-Biphenyl-3-yl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-(9H-Fluoren-1-yl)-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-(4-Benzyloxy-phenyl)-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-Benzyl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-phenethyl-acetamide;
1-{1-[3-(3-{3-[2-(3-Hydroxy-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-pyrrolidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxymethyl-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxy-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(3-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(octahydro-isoquinolin-2-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Fluoro-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
(S)-1-{1-[3-(5-Methanesulfonyl-3-{3-[2-oxo-2-(2-trifluoromethyl-pyrrolidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
N-(4-Fluoro-phenyl)-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetamide;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-N,N-dimethyl-acetamide;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-N-methyl-acetamide;
1-{1-[3-(3-{3-[2-(4-Dimethylamino-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Dimethylamino-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[3-(2-[1,4′]Bipiperidinyl-1′-yl-2-oxo-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetyl}-piperidine-4-carboxylic acid amide;
1-[1-(3-{5-Methanesulfonyl-3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-oxo-2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-[5-Methanesulfonyl-3-[3-(2-morpholin-4-yl-2-oxo-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{3-[3-(2-Azepan-1-yl-2-oxo-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-methyl-[1,4]diazepan-1-yl)-2-oxo-ethylsulfanyl]-4-trifluoromethyl-phenyl}-4,5,6,7-tetrahydro-pyrazolo[4,3c]pyridin-1-yl)-propyl]-piperidin-4-yl}-pyrrolidin-2-one;
1-{1-[3-(3-{3-[2-(3,6-Dihydro-2H-pyridin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,4-Dihydro-1H-isoquinolin-2-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethylphenylsulfanyl]-N-methyl-N-(1-methyl-piperidin-4-yl)-acetamide;
1-{1-[3-(5-Methanesulfonyl-3-{3-[2-oxo-2-(4-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
N-Cyclohexyl-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethylphenylsufanyl]-N-methyl-acetamide;
N-Benzyl-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetamide;
1-{1-[3-(5-Methanesulfonyl-3-{3-[2-(4-methyl-piperazin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Benzyl-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-oxo-2-(4-phenethyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-oxo-2-(4-phenyl-3,6-dihydro-2H-pyridin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl phenylsulfanyl]-acetyl}-piperidine-4-carboxylic acid diethylamide;
(R)-1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetyl}-pyrrolidine-2-carboxylic acid amide;
1-{1-[3-(3-{3-[2-(4-Isopropyl-piperazin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
2-(5-{5-Methanesulfonyl-1-[3-(4-pyrrolidin-1-yl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone;
2-(5-{5-Methanesulfonyl-1-[3-(4-trifluoromethyl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone;
2-(5-{1-[3-(4-Acetyl-piperazin-1-yl)-propyl]-5-methanesulfonyl-4,5,6,7-tetrahydro-1H-pyrazolo[4,3-c]pyridin-3-yl}-2-trifluoromethyl-phenylsulfanyl)-1-morpholin-4-yl-ethanone;
[1-(3-{5-Methanesulfonyl-3-[3-(2-morpholin-4-yl-2-oxo-ethylsulfanyl)-4-trifluoromethyl-phenyl]-4,5,6,7-tetrahydro-pyrazolo[4,3-c]pyridin-1-yl}-propyl)-piperidin-4-yl]-carbamic acid tert-butyl ester;
N-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
N-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-2-phenyl-acetamide;
4-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-piperidine-1-carboxylic acid tert-butyl ester;
({2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-methyl)-carbamic acid tert-butyl ester;
4-Fluoro-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
4-Hydroxy-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
4-Dimethylamino-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
2-(4-Fluoro-phenyl)-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-acetamide;
3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-piperidine-1-carboxylic acid tert-butyl ester;
3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-pyrrolidine-1-carboxylic acid tert-butyl ester;
(3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-propyl)-carbamic acid tert-butyl ester;
Piperidine-3-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
Pyrrolidine-3-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
Piperidine-4-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
(2S)-1-(4,4-Dimethyl-piperidin-1-yl)-3-(3-{3-[2-(4-fluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propan-2-ol;
(2S)—N-{1-[3-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-acetamide; and
(2S)-1-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-3-(4-methylpiperidin-1-yl)-propan-2-ol;
and pharmaceutically acceptable salts thereof.

55. A compound selected from the group consisting of:
1-[1-(3-{3-[3-(2-Amino-ethylsulfanyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-pyrrolidin-2-one;
1-[1-(3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
N-[1-(3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-acetamide;
5-Dimethylamino-3-[1-(3-{3-[3-(2-hydroxy-ethylsulfanyl)-4-trifluoromethyl-phenyl]-5-methanesulfonyl-4,5,6,7-tetrahydro-pyrazolo[4,3-c]pyridin-1-yl}-propyl)piperidin-4-yl]-1-methyl-1,3-dihydro-imidazo[4,5-b]pyridin-2-one;
[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid ethyl ester;
[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid;
3-(3-Benzylsulfanyl-4-chloro-phenyl)-5-methanesulfonyl-1-(3-morpholin-4-yl-propyl)-4,5,6,7-tetrahydro-1H-pyrazolo[4,3-c]pyridine;
3-(4-Chloro-3-phenethylsulfanyl-phenyl)-5-methanesulfonyl-1-(3-morpholin-4-yl-propyl)-4,5,6,7-tetrahydro-1H-pyrazolo[4,3-c]pyridine;
5-Methanesulfonyl-1-(3-morpholin-4-yl-propyl)-3-[3-(2-phenoxy-ethylsulfanyl)-4-trifluoromethyl-phenyl]-4,5,6,7-tetrahydro-1H-pyrazolo[4,3-c]pyridine;
3-[4-Chloro-3-(furan-2-ylmethylsulfanyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-propylsulfanyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(2-phenoxy-ethylsulfanyl)-phenyl]-5-methanesulfonyl-1-(3-morpholin-4-yl-propyl)-4,5,6,7-tetrahydro-1H-pyrazolo[4,3-c]pyridine;
1-(3-{3-[3-(4-Chloro-benzylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
3-[4-Chloro-3-(4-chloro-benzylsulfanyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-fluoro-benzylsulfanyl)-phenyl]-5-methanesulfonyl-1-(3-morpholin-4-yl-propyl)-4,5,6,7-tetrahydro-1H-pyrazolo[4,3-c]pyridine;
1-[1-(3-{5-Methanesulfonyl-3-[3-(2-phenoxy-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
3-[4-Chloro-3-(2-pyrazin-2-yl-ethylsulfanyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-chloro-propylsulfanyl)-phenyl]-5-methanesulfonyl-1-(3-morpholin-4-yl-propyl)-4,5,6,7-tetrahydro-1H-pyrazolo[4,3-c]pyridine; and
3-(4-Chloro-3-phenylmethanesulfinyl-phenyl)-5-methanesulfonyl-1-(3-morpholin-4-yl-propyl)-4,5,6,7-tetrahydro-1H-pyrazolo[4,3-c]pyridine;
and pharmaceutically acceptable salts thereof.

56. A compound selected from the group consisting of:
1-[1-(3-{3-[3-({2-[(1,1-Dimethylethyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
3-[3-({2-[(3S)-3-Methylmorpholin-4-yl]ethyl}sulfanyl)-4-(trifluoromethyl)phenyl]-5-(methylsulfonyl)-1-(3-piperidin-1-ylpropyl)-4,5,6,7-tetrahydro-1H-pyrazolo[4,3-c]pyridine;
3-[3-({2-[(2R,6R)-2,6-Dimethylmorpholin-4-yl]ethyl}sulfanyl)-4-(trifluoromethyl)phenyl]-5-(methylsulfonyl)-1-(3-piperidin-1-ylpropyl)-4,5,6,7-tetrahydro-1H-pyrazolo[4,3-c]pyridine;
{4-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]morpholin-2-yl}methanol;
{(3S)-4-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]morpholin-3-yl}methanol;
5-(Dimethylamino)-1-methyl-3-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-imidazo[4,5-b]pyridin-2-one;
Ethyl(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
N-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]propan-1-amine;
N-[2-({5-[5-(Methylsulfonyl)-1-{3-[4-(piperidin-1-ylcarbonyl)piperidin-1-yl]propyl}-4,5,6,7-tetrahydro-1H-pyrazolo[4,3-c]pyridin-3-yl]-2-(trifluoromethyl)phenyl}sulfanyl)ethyl]propan-1-amine;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
5-(Dimethylamino)-3-(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1-methyl-1,3-dihydro-2H-imidazo[4,5-b]pyridin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
2-(4-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-(4-Cyclopropylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-morpholin-4-ylpropan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-pyrrolidin-1-ylpropan-2-ol;
1-[4-(Dimethylamino)piperidin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-(4-Methylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-({2-[2-(Hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-[1-(2-Hydroxy-3-{3-[3-({2-[2-(hydroxymethyl)morpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
4-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl}sulfanyl)ethyl]-3-methylpiperazin-2-one;
4-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]-3-methylpiperazin-2-one;
4-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)-3-methylpiperazin-2-one;
1-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol;
1-[1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
2-[4-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperazin-1-yl]benzonitrile;
1-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-ol;
5-(Dimethylamino)-3-[1-(2-hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-(4-Methylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4,4-Dimethylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4-Fluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(1,1-Dimethylethyl)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-morpholin-4-ylpropan-2-ol;
5-(Dimethylamino)-3-(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1-methyl-1,3-dihydro-2H-imidazo[4,5-b]pyridin-2-one;
1-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
2-(4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{5-(Ethylsulfonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-3-ol;
1-(2-{[5-{1-[2-Hydroxy-3-(4-hydroxy-4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-3-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(2-hydroxyethyl)piperidin-1-yl]propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxamide;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-2-hydroxyacetamide;
1-{1-[(2S)-2-Hydroxy-3-{3-[3-({2-[(2R)-2-(hydroxymethyl)morpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[(2S)-2-Hydroxy-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]-4-methylpiperidin-4-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(octahydroisoquinolin-2(1H)-yl)propan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrazin-2-ylpiperazin-1-yl)propan-2-ol;
(2R)-1-[4-(3,5-Dichloropyridin-4-yl)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(3,6-Dihydropyridin-1(2H)-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-[4-(1,1-Dimethylethyl)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
N-{1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-dimethylethyl)piperidin-1-yl]propan-2-ol;
(2S)-1-(4-Cyclohexylpiperidin-1-yl)-3-{3-[3-{[2-(4,4-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(Hexahydrocyclopenta[c]pyrrol-2(1H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-methylpiperidin-4-ol;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidine-4-carboxamide;
(2S)-1-{3-[3-({2-[4-(1,1-Dimethylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
N-[1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-yl]acetamide;
2-Hydroxy-N-[1-(2-{[5-{1-[(2S)-2-hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-yl]acetamide;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-pyridin-2-ylpiperidin-4-ol;
(2S)-1-{3-[3-({2-[(3S)-3-Methylmorpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(2S)-1-{3-[3-{[2-(Hexahydrocyclopenta[c]pyrrol-2(1H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-methylpiperidin-4-ol;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidine-4-carboxamide;
(2S)-1-{3-[3-({2-[4-(1-Hydroxy-1-methylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
5-(Dimethylamino)-3-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-1-methyl-1,3-dihydro-2H-imidazo[4,5-b]pyridin-2-one;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyrrolidin-1-ylpiperidin-1-yl)propan-2-ol;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-(1-{2-Hydroxy-3-[5-(phenylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-(1-{3-[5-(Ethylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-2-hydroxypropyl}piperidin-4-yl)pyrrolidin-2-one;
1-[5-(Ethylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-{1-[2-Hydroxy-3-(5-[(phenylmethyl)sulfonyl]-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-[5-(Hydroxyacetyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-[5-(Hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-(1-{2-Hydroxy-3-[5-(hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)piperidin-4-yl]pyrrolidin-2-one;
1-[3-{3-[(2-Morpholin-4-ylethyl)sulfanyl]-4-(trifluoromethyl)phenyl}-5-(piperidin-4-ylacetyl)-4,5,6,7-tetrahydro-1H-pyrazolo[4,3-c]pyridin-1-yl]-3-piperidin-1-ylpropan-2-ol;
Methyl[1-(2-hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl](oxo)acetate;
Methyl(1-{2-hydroxy-3-[4-(2-oxopyrrolidin-1-yl)piperidin-1-yl]propyl}-3-{3-[(2-piperidin-1-ylethyl)sulfanyl]-4-(trifluoromethyl)phenyl}-1,4,6,7-tetrahydro-5H-pyrazolo[4,3-c]pyridin-5-yl)(oxo)acetate;
1-(5-Acetyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-{1-[3-(5-Acetyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}pyrrolidin-2-one;
1-(1-{3-[5-(Furan-2-ylcarbonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-2-hydroxypropyl}piperidin-4-yl)pyrrolidin-2-one;
1-(5-Methyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
2-(1-{(2S)-3-[4-(Acetylamino)piperidin-1-yl]-2-hydroxypropyl}-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
2-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-2-oxoacetamide;
(2S)-1-{3-[3-{[2-(4-Fluoropiperidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-piperidin-1-ylpropan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-methyl-1,4,6,7-tetrahydro-5H-pyrazolo[4,3-c]pyridine-5-carboxamide;
5-(Methylsulfonyl)-3-{3-[(2-morpholin-4-yl-2-oxoethyl)sulfanyl]-4-(trifluoromethyl)phenyl}-1-(3-piperidin-1-ylpropyl)-4,5,6,7-tetrahydro-1H-pyrazolo[4,3-c]pyridine;
5-(Methylsulfonyl)-3-{3-[(2-morpholin-4-yl-2-oxoethyl)sulfanyl]-4-(trifluoromethyl)phenyl}-1-(3-morpholin-4-ylpropyl)-4,5,6,7-tetrahydro-1H-pyrazolo[4,3-c]pyridine;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-morpholin-4-ylpiperidin-1-yl)propan-2-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-{4-[2-(Methyloxy)phenyl]piperidin-1-yl}-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propan-2-ol;
1-{[1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-[4-(1,1-Dimethylethyl)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyridin-2-ylpiperidin-1-yl)propan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-{4-[2-(methyloxy)phenyl]piperidin-1-yl}propan-2-ol;
1-{3-[3-{[2-(4-Fluoropiperidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-(4-pyridin-2-ylpiperidin-1-yl)propan-2-ol;
1-(5-Acetyl-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)-3-{4-[2-(methyloxy)phenyl]piperidin-1-yl}propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(1,1-dimethylethyl)piperidin-1-yl]propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-methylpiperidin-1-yl)propan-2-ol;
1-{1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}pyrrolidin-2-one; and
1-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
and pharmaceutically acceptable salts thereof.

57. A compound selected from the group consisting of:
1-[3-{3-[2-(4-Fluoro-piperidin-1-yl)-ethylsulfanyl]-4-trifluoromethyl-phenyl}-1-(2-hydroxy-3-piperidin-1-yl-propyl)-1,4,6,7-tetrahydro-pyrazolo[4,3-c]pyridin-5-yl]-2-hydroxy-ethanone;
(2S)-1-(4-Cyclohexyl-piperidin-1-yl)-3-(3-{3-[2-((3S)-fluoro-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propan-2-ol;
N-[1-(2-{5-[1-((2S)-Hydroxy-3-morpholin-4-yl-propyl)-5-methanesulfonyl-4,5,6,7-tetrahydro-1H-pyrazolo[4,3-c]pyridin-3-yl]-2-trifluoromethyl-phenylsulfanyl}-ethyl)-piperidin-4-yl]-acetamide;
(2S)-1-(3-{3-[2-((3S)-Fluoro-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-3-(3′,4′,5′,6′-tetrahydro-2′H-[2,4′]bipyridinyl-1′-yl)-propan-2-ol;
(3S)-1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-3-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-pyrrolidin-1-ylpropan-2-ol;
1-Piperidin-1-yl-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]pyrrolidin-3-ol;
1-(3-{3-[(2-Morpholin-4-ylethyl)sulfanyl]-4-(trifluoromethyl)phenyl-4,5,6,7-tetrahydro-1H-pyrazolo[4,3-c]pyridin-1-yl)-3-piperidin-1-ylpropan-2-ol;
1-Morpholin-4-yl-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(4-Methylpiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-ol;
1-[2-({5-[1-(2-Hydroxy-3-piperidin-1-ylpropyl)-4,5,6,7-tetrahydro-1H-pyrazolo[4,3-c]pyridin-3-yl]-2-(trifluoromethyl)phenyl}sulfanyl)ethyl]piperidin-3-ol;
1-(4-Fluoropiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-morpholin-4-ylpropan-2-ol;
1-(Hexahydrocyclopenta[c]pyrrol-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol
1-(4,4-Dimethylpiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]-4-methylpiperidin-4-ol;
1-[4-(Methyloxy)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(4-Fluoropiperidin-1-yl)ethyl]sulfanyl}-4-(trifluoromethyl)phenyl]-5-methyl-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-methylpiperidin-1-yl)propan-2-ol;
1-(1,3-Dihydro-2H-isoindol-2-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
3-{3-[(2-Azetidin-1-ylethyl)sulfanyl]-4-(trifluoromethyl)phenyl}-5-(methylsulfonyl)-1-(3-piperidin-1-ylpropyl)-4,5,6,7-tetrahydro-1H-pyrazolo[4,3-c]pyridine;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidine-4-carboxamide;
1-(4-Acetylpiperazin-1-yl)-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidine-4-carboxamide;
1-(3,3-Difluoropyrrolidin-1-yl)-3-{3-[3-{[2-(3,3-difluoropyrrolidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-(4,4-Dimethylpiperidin-1-yl)-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
5-(Methylsulfonyl)-1-(3-piperidin-1-ylpropyl)-3-{3-[(2-pyrrolidin-1-ylethyl)sulfanyl]-4-(trifluoromethyl)phenyl}-4,5,6,7-tetrahydro-1H-pyrazolo[4,3-c]pyridine;
1-(3,4-Dihydroisoquinolin-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
N-Methyl-N-[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]propan-2-amine;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[(3S)-3-(trifluoromethyl)pyrrolidin-1-yl]propan-2-ol;
1-(Octahydroisoquinolin-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
2-(1-[(2S)-2-Hydroxy-3-piperidin-1-ylpropyl]-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
1-(1,4-Dioxa-8-azaspiro[4.5]dec-8-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[4-(1-Hydroxy-1-methylethyl)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}urea;
(2S)-1-{5-Acetyl-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidine-4-carboxamide;
1-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]imidazolidin-4-one;
1-{3-[3-{[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
5-(Methylsulfonyl)-3-{3-[(2-morpholin-4-ylethyl)sulfanyl]-4-(trifluoromethyl)phenyl}-1-(3-piperidin-1-ylpropyl)-4,5,6,7-tetrahydro-1H-pyrazolo[4,3-c]pyridine;
1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidine-4-carboxamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-pyrrolidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(hexahydrocyclopenta[c]pyrrol-2(1H)-yl)propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4,4-dimethylpiperidin-1-yl)propan-2-ol;
1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidine-4-carboxamide;
2-Hydroxy-N-{1-[2-hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
2-Hydroxy-N-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
(2S)-1-[1-(2-Hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl]-1-oxopropan-2-ol;
1-(5-[(Methyloxy)acetyl]-3-{3-[(2-morpholin-4-ylethyl)sulfanyl]-4-(trifluoromethyl)phenyl]-4,5,6,7-tetrahydro-1H-pyrazolo[4,3-c]pyridin-1-yl)-3-piperidin-1-ylpropan-2-ol;
1-[2-({5-[5-(Hydroxyacetyl)-1-(2-hydroxy-3-piperidin-1-ylpropyl)-4,5,6,7-tetrahydro-1H-pyrazolo[4,3-c]pyridin-3-yl]-2-(trifluoromethyl)phenyl}sulfanyl)ethyl]piperidin-3-ol;
(2S)-1-{3-[3-{[2-(4-Fluoropiperidin-1-yl)ethyl]sulfanyl}-4-(trifluoromethyl)phenyl]-5-propanoyl-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-glycyl-4,5,6,7-tetrahydro-1H-pyrazolo[4,3-c]pyridin-1-yl}-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(1,3-dihydro-2H-isoindol-2-yl)propan-2-ol;
N-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]cyclohexanamine;
1-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}urea;
N-(2-{[5-{1-[3-(4-Cyclopropylpiperazin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)propan-1-amine;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-4-ylpiperidin-1-yl)propan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
1-(4-Methylpiperidin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(4-Methylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}pyrrolidin-3-ol;
1-[(3S)-3-(Hydroxymethyl)pyrrolidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[(3S)-3-Methylmorpholin-4-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]urea;
(2S)-1-(5,6-Dihydropyrimidin-1(4H)-yl)-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-morpholin-4-ylpropan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}pyrrolidin-3-ol;
5-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-2,4-dihydro-3H-1,2,4-triazol-3-one;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
2-{1-[(2S)-2-Hydroxy-3-piperidin-1-ylpropyl]-3-[3-{[2-(4-methylpiperidin-1-yl)ethyl]sulfanyl}-4-(trifluoromethyl)phenyl]-1,4,6,7-tetrahydro-5H-pyrazolo[4,3-c]pyridin-5-yl}-2-oxoacetamide;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-(4-pyrrolidin-1-ylpiperidin-1-yl)propan-2-ol;
1-(4-Morpholin-4-ylpiperidin-1-yl)-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
2-{1-(2-Hydroxy-3-piperidin-1-ylpropyl)-3-[3-{[2-(4-methylpiperidin-1-yl)ethyl]sulfanyl}-4-(trifluoromethyl)phenyl]-1,4,6,7-tetrahydro-5H-pyrazolo[4,3-c]pyridin-5-yl}-2-oxoacetamide;
N-{1-[(2S)-3-(5-Acetyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}acetamide;
(4R)-1-{1-[(2S)-3-(5-Acetyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}-4-hydroxypyrrolidin-2-one;
Ethyl {1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}carbamate;
N-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-dimethyl-1,4,6,7-tetrahydro-5H-pyrazolo[4,3-c]pyridine-5-carboxamide;
1-(5-[(Methylsulfanyl)acetyl]-3-{3-[(2-morpholin-4-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(3,4-dihydroisoquinolin-2(1H)-yl)propan-2-ol;
(2S)-1-{3-[3-{[2-(4-Fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-1-oxopropan-2-ol;
(2S)-1-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-1-oxopropan-2-ol;
1-{3-[3-{[2-(4-Fluoropiperidin-1-yl)ethyl]sulfanyl}-4-(trifluoromethyl)phenyl]-5-[(methyloxy)acetyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-[(methyloxy)acetyl]-4,5,6,7-tetrahydro-1H-pyrazolo[4,3-c]pyridin-1-yl}-3-piperidin-1-ylpropan-2-ol;
3-Methyl-4-[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]piperazin-2-one;
N-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}-2-hydroxyacetamide;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(4,4-Dimethylpiperidin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
2-{4-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
N-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
1-(4-Methyl-1,4-diazepan-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
3-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
4-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
2-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}-4-methylpiperidin-4-ol;
(2S)-1-(3,6-Dihydropyridin-1(2H)-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
(3S)-1-[({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
(3R)-1-[({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(2S)-1,3-[3-({2-[(3S)-3-Methylmorpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(3S)-1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-3-ol;
(2S)-1-(5,6-Dihydropyrimidin-1(4H)-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(5,6-Dihydropyrimidin-1(4H)-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-(4-Methylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(4-Fluorophenyl)piperazin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
N-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]-2-hydroxyacetamide;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-4-ylpiperidin-1-yl)propan-2-ol;
1-[4-(2-Fluorophenyl)piperazin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
1-[4-(4-Fluorophenyl)piperazin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-Azepan-1-yl-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
(3R)-1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-3-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-ol;
(3S)-1-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}pyrrolidin-3-ol;
(3R)-1-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}pyrrolidin-3-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(3S)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-ol;
N-Methyl-2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)-N-(phenylmethyl)ethanamine;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperidin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-[1-(2-Hydroxy-3-{3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
(4R)-4-Hydroxy-1-{1-[(2S)-2-hydroxy-3-(5-methyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(1-hydroxy-1-methylethyl)piperidin-1-yl]propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(1,4-dioxa-8-azaspiro[4.5]dec-8-yl)propan-2-ol;
(2S)-1-{5-(Cyclobutylcarbonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
4-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
3-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
4-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
1-{5-[(1-Aminocyclopropyl)carbonyl]-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}pyrrolidin-2-one;
N-(2-{[5-{1′-[2-Hydroxy-3-(4-methylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
2-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}benzonitrile;
2-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-N-methyl-2-oxoacetamide;
1-{5-(N,N-Dimethylglycyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-(1-methylethyl)-1,4,6,7-tetrahydro-5H-pyrazolo[4,3-c]pyridine-5-carboxamide;
Methyl {3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}(oxo)acetate;
Methyl {3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}(oxo)acetate;
1-{3-[3-{[2-(4-Fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-methyl-1-oxopropan-2-ol;
1-(4-Cyclopropylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]pyrrolidin-2-one;
N-(2-{[5-{5-(Methylsulfonyl)-1-[3-(4-pyrrolidin-1-ylpiperidin-1-yl)propyl]-4,5,6,7-tetrahydro-1H-pyrazolo[4,3-c]pyridin-3-yl}-2-(trifluoromethyl)phenyl]sulfanyl}ethyl)propan-1-amine;
(2S)-1-{3-[3-{[2-(4-Fluoropiperidin-1-yl)ethyl]sulfanyl}-4-(trifluoromethyl)phenyl]-5-[(methylsulfanyl)acetyl]-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-[(methylsulfanyl)acetyl]-4,5,6,7-tetrahydro-1H-pyrazolo[4,3-c]pyridin-1-yl}-3-piperidin-1-ylpropan-2-ol;
1-{4-[2-(Methyloxy)phenyl]piperidin-1-yl}-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-(4-Methylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-Azepan-1-yl-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{4-[4-(Methyloxy)phenyl]piperidin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[2-(Methyloxy)phenyl]piperidin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
1-(4-Acetylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}pyrrolidin-3-yl)acetamide;
1-{4-[3-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[4-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[2-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}praline;
(2S)-1-[(3S)-3-Methylmorpholin-4-yl]-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(3,3-Dimethylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-3-methylpiperazin-2-one;
N-{1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]-4-pyridin-2-ylpiperidin-4-ol;
1-(2-{[5-{1-[2-Hydroxy-3-(4-hydroxy-4-pyridin-2-ylpiperidin-1-yl)propyl]-4,5,6,7-tetrahydro-1H-pyrazolo[4,3-c]pyridin-3-yl}-2-(trifluoromethyl)phenyl]sulfanyl}ethyl)piperidin-3-ol;
N-[1-(2-Hydroxy-3-{3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
(2S)-1-(4,4-Dimethylpiperidin-1-yl)-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
1-[(3S)-3-(Hydroxymethyl)pyrrolidin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-[(3S)-3-Methylmorpholin-4-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
1-[4-(3-Chlorophenyl)piperidin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[4-(Methyloxy)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-Azepan-1-yl-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-Azepan-1-yl-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
1-(2-{[5-(1-{2-Hydroxy-3-[4-(pyrimidin-2-yloxy)piperidin-1-yl]propyl}-4,5,6,7-tetrahydro-1H-pyrazolo[4,3-c]pyridin-3-yl)-2-(trifluoromethyl)phenyl]sulfanyl}ethyl)piperidin-3-ol;
1-(4-Aminopiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
N-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}methanesulfonamide;
2-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}phenol;
3-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}phenol;
1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-ol;
(3S)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-3-ol;
1-[2-(Hydroxymethyl)morpholin-4-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-3-ol;
1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(3,6-dihydropyridin-1(2H)-yl)propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-3-ol;
(3R)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-3-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-ol;
N-Methyl-2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)-N-(2-phenylethyl)ethanamine;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[(2S)-2-(hydroxymethyl)pyrrolidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(5,6-dihydropyrimidin-1(4H)-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(5,6-dihydropyrimidin-1(4H)-yl)propan-2-ol;
(2S)-1-{3-[3-{[2-(4-Fluoropiperidin-1-yl)ethyl]sulfanyl}-4-(trifluoromethyl)phenyl]-5-(1H-1,2,4-triazol-3-ylcarbonyl)-4,5,6,7-tetrahydro-1H-pyrazolo[4,3-c]pyridin-1-yl}-3-piperidin-1-ylpropan-2-ol;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
1-[4-(Morpholin-4-ylcarbonyl)piperidin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(2-{[5-(1-{2-Hydroxy-3-[4-(pyrrolidin-1-ylcarbonyl)piperidin-1-yl]propyl}-4,5,6,7-tetrahydro-1H-pyrazolo[4,3-c]pyridin-3-yl)-2-(trifluoromethyl)phenyl}sulfanyl]ethyl)piperidin-3-ol;
(2S)-1-[4-(2-Fluorophenyl)piperazin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-[4-(4-Fluorophenyl)piperazin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-(3-Fluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-(4-Fluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(4-Fluoropiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
N-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]methanesulfonamide;
4-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]phenol;
1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)-4-pyridin-2-ylpiperidin-4-ol;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
3-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]phenol;
2-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]pyridine-3-carbonitrile;
(2S)-1-{3-[3-{[2-(4-Fluoropiperidin-1-yl)ethyl]sulfanyl}-4-(trifluoromethyl)phenyl]-5-(pyrrolidin-1-ylcarbonyl)-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
N-(2-{[5-{1-[3-(4,4-Dimethylpiperidin-1-yl)-2-hydroxypropyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-(4,4-Difluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(3,3-Difluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
1-{3-[3-{[2-(4-Fluoropiperidin-1-yl)ethyl]sulfanyl}-4-(trifluoromethyl)phenyl]-5-[(2R)-tetrahydrofuran-2-ylcarbonyl]-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(4-fluorophenyl)piperazin-1-yl]propan-2-ol;
N-(1,1-Dimethylethyl)-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-(1-methylpropyl)-1,4,6,7-tetrahydro-5H-pyrazolo[4,3-c]pyridine-5-carboxamide;
1-[1-(3-{3-[3-{[2-(Cyclopropylamino)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
N,N-Diethyl-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
2-{4-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperazin-1-yl}benzonitrile;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(2,4,6,7-tetrahydro-5H-pyrazolo[4,3-c]pyridin-5-yl)propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(1,4,6,7-tetrahydro-5H-imidazo[4,5-c]pyridin-5-yl)propan-2-ol;
N-(1-{3-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
1-[1-(3-{3-[3-({2-[(1-Methylethyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-{[2-(propylamino)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
4-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}benzonitrile;
1-(4-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenyl)ethanone;
N,N-Dimethyl-1-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-3-yl)methanamine;
3-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}benzonitrile;
2-[4-(3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]benzonitrile;
2-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}benzonitrile;
1-[4-(1,1-Dimethylethyl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-(4,4-Dimethylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{4-[2-(Methyloxy)phenyl]piperidin-1-yl}-3-(5-methyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(4-Cyclopropylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4-Acetylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
2-{4-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperazin-1-yl}phenol;
1-[4-(1-Methylethyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{4-[2-(Ethyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{5-(Cyclopropylsulfonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[4-(1-Hydroxy-1-methylethyl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]-4-methylpiperidin-4-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
N-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}pyrrolidin-3-yl)acetamide;
1-(4-Acetylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(Dimethylamino)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(1-Methylethyl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidine-4-carboxamide;
1-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(4-fluorophenyl)piperazin-1-yl]propan-2-ol;
(2S)-1-{3-[3-{[2-(4-Fluoropiperidin-1-yl)ethyl]sulfanyl}-4-(trifluoromethyl)phenyl]-5-(propylsulfonyl)-4,5,6,7-tetrahydro-1H-pyrazolo[4,3-c]pyridin-1-yl}-3-piperidin-1-ylpropan-2-ol
(2S)-1-{3-[3-{[2-(4-Fluoropiperidin-1-yl)ethyl]sulfanyl}-4-(trifluoromethyl)phenyl]-5-[(1-methylethyl)sulfonyl]-4,5,6,7-tetrahydro-1H-pyrazolo[4,3-c]pyridin-1-yl}-3-piperidin-1-ylpropan-2-ol;
(2S)-1-(4,4-Dimethylpiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
2-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]benzonitrile;
(2R)-1-(4,4-Dimethylpiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-(4,4-Dimethylpiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(methyloxy)phenyl]piperidin-1-yl}propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[4-(methyloxy)phenyl]piperidin-1-yl}propan-2-ol;
1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-dimethyl-1,4,6,7-tetrahydro-5H-pyrazolo[4,3-c]pyridine-5-sulfonamide;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(methyloxy)phenyl]piperazin-1-yl}propan-2-ol;
1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4-Fluoropiperidin-1-yl)ethyl]sulfanyl}-4-(trifluoromethyl)phenyl]-5-[(1-methyl-1H-imidazol-2-yl)carbonyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(hydroxymethyl)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(hydroxymethyl)piperidin-1-yl]propan-2-ol;
1-(5-Acetyl-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl)-3-(4-pyrrolidin-1-ylpiperidin-1-yl)propan-2-ol;
1-(3,4-Dihydroisoquinolin-2(1H)-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-Azepan-1-yl-3-{3-[3-{[2-(3,3-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-Azepan-1-yl-3-{3-[3-{[2-(4,4-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(2-thienylcarbonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(2-thienylcarbonyl)-4,5,6,7-tetrahydro-1H-pyrazolo[4,3-c]pyridin-1-yl}-3-piperidin-1-ylpropan-2-ol;
(2S)-1-[4-(3-Chlorophenyl)piperidin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
N-[1-(3-{5-(Methylsulfonyl)-3-[3-{[2-(propylamino)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[3-(methyloxy)phenyl]piperazin-1-yl}propan-2-ol;
1-{3-[3-{[2-(4-Fluoropiperidin-1-yl)ethyl]sulfanyl}-4-(trifluoromethyl)phenyl]-5-(1-methyl-L-prolyl)-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[4-(methyloxy)phenyl]piperazin-1-yl}propan-2-ol;
1-{3-[3-{[2-(4-Fluoropiperidin-1-yl)ethyl]sulfanyl}-4-(trifluoromethyl)phenyl]-5-(piperidin-3-ylcarbonyl)-4,5,6,7-tetrahydro-1H-pyrazolo[4,3-c]pyridin-1-yl}-3-piperidin-1-ylpropan-2-ol;
4-{1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}benzonitrile;
2-Hydroxy-N-{1-[2-hydroxy-3-(5-[(methyloxy)acetyl]-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
(2S)-1-{3-[3-{[2-(4-Fluoropiperidin-1-yl)ethyl]sulfanyl}-4-(trifluoromethyl)phenyl]-5-(morpholin-4-ylcarbonyl)-4,5,6,7-tetrahydro-1H-pyrazolo[4,3-c]pyridin-1-yl}-3-piperidin-1-ylpropan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-(4,4-Difluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{5-(Methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[1-(3-{3-[3-({2-[(2-Methylpropyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-pyrrolidin-1-ylpropan-2-ol;
(2S)-1-{5-(Methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}-3-morpholin-4-ylpropan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(octahydroisoquinolin-2(1H)-yl)propan-2-ol;
N-[(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methyl]acetamide;
1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidine-4-carboxamide;
1-[1-(3-{3-[3-({2-[(3-Hydroxypropyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[5-(Hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)urea;
1-(1,5-Dioxa-9-azaspiro[5.5]undec-9-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-{3-[3-({2-[4-(1-Hydroxy-1-methylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
2-Hydroxy-N-(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-phenyl-1,4,6,7-tetrahydro-5H-pyrazolo[4,3-c]pyridine-5-carboxamide;
(2S)-1-[4-(1,1-Dimethylethyl)piperidin-1-yl]-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
N-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
(2S)-1-[4-(Dimethylamino)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
(2S)-1-{4-[2-(Ethyloxy)phenyl]piperazin-1-yl}-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxylic acid;
(3S)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-3-carboxylic acid;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-3-carboxylic acid;
(3R)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-3-carboxylic acid;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-phenylpiperidin-1-yl)propan-2-ol;
1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-3-carboxylic acid;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-yl]acetamide;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(2-hydroxyethyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4,4-dimethylpiperidin-1-yl)propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4,4-dimethylpiperidin-1-yl)propan-2-ol;
N-[1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-yl]acetamide;
1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-2-thienyl-1,4,6,7-tetrahydro-5H-pyrazolo[4,3-c]pyridine-5-carboxamide;
1-{3-[3-{[2-(4-Fluoropiperidin-1-yl)ethyl]sulfanyl}-4-(trifluoromethyl)phenyl]-5-(piperidin-4-ylacetyl)-4,5,6,7-tetrahydro-1H-pyrazolo[4,3-c]pyridin-1-yl}-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(ethyloxy)phenyl]piperazin-1-yl}propan-2-ol;
N-Cyclohexyl-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4-Fluoropiperidin-1-yl)ethyl]sulfanyl}-4-(trifluoromethyl)phenyl]-5-[(4-methylpiperazin-1-yl)carbonyl]-4,5,6,7-tetrahydro-1H-pyrazolo[4,3-c]pyridin-1-yl}-3-piperidin-1-ylpropan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-{4-[3-(trifluoromethyl)phenyl]piperazin-1-yl}propan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-bis(1-methylethyl)-1,4,6,7-tetrahydro-5H-pyrazolo[4,3-c]pyridine-5-carboxamide;
(2S)-1-[4-(1,1-Difluoroethyl)piperidin-1-yl]-3-{3-[3-({2-[(3S)-3-fluoropyrrolidin-1-yl]ethylsulfanyl)-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(4-Methylpiperidin-1-yl)-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propan-2-ol;
2-Hydroxy-N-{1-[2-hydroxy-3-(5-[(methylsulfanyl)acetyl]-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
1-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-(1,4′-Bipiperidin-1′-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(trifluoromethyl)piperidin-1-yl]propan-2-ol;
(2S)-1-[4-(1,1-Dimethylethyl)piperidin-1-yl]-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol;
N-{1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}acetamide;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
(2S)-1-{3-[3-{[2-(4-Fluoropiperidin-1-yl)ethyl]sulfanyl}-4-(trifluoromethyl)phenyl]-5-[(trifluoromethyl)sulfonyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(octahydroquinolin-1(2H)-yl)propan-2-ol;
(2S)-1-[4-(1-Hydroxy-1-methylethyl)piperidin-1-yl]-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
N-[(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)methyl]acetamide;
Ethyl(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
(2S)-1-[4-(1,1-Dimethylethyl)piperidin-1-yl]-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(4-Fluoropiperidin-1-yl)ethyl]sulfanyl}-4-(trifluoromethyl)phenyl]-5-[(2S)-2-hydroxy-2-phenylacetyl]-4,5,6,7-tetrahydro-1H-pyrazolo[4,3-c]pyridin-1-yl}-3-piperidin-1-ylpropan-2-ol;
2-Hydroxy-N-[1-(2-{[5-{1-[(2S)-2-hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-yl]acetamide;
N-{1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
2-Hydroxy-N-(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
N-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-phenylpiperazin-1-yl)propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-4-ylpiperidin-1-yl)propan-2-ol;
1-(5-Acetyl-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-phenylpiperidin-1-yl)propan-2-ol;
2-(1-[(2S)-2-Hydroxy-3-{4-[(4R)-4-hydroxy-2-oxopyrrolidin-1-yl]piperidin-1-yl}propyl]-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyridin-4-ylpiperidin-1-yl)propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-phenylpiperazin-1-yl)propan-2-ol;
N-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
1-[1-(2-Hydroxy-3-{5-(hydroxyacetyl)-3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
1-{5-(1-Acetyl-L-prolyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyrimidin-2-ylpiperazin-1-yl)propan-2-ol;
1-[1-(3-{3-[3-{[2-(Cyclohexylamino)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]-4-[3-(trifluoromethyl)phenyl]piperidin-4-ol;
2-(4-{2-Hydroxy-3-[5-(hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
5-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-Hydroxy-1-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[4-(3-Methyl-1,2,4-oxadiazol-5-yl)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-cyclopropylpiperazin-1-yl)propan-2-ol;
5-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-Hydroxy-1-(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyrrolidin-1-ylpiperidin-1-yl)propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[3-(trifluoromethyl)phenyl]piperazin-1-yl}propan-2-ol;
N-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
Ethyl(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
N-(1-{2-Hydroxy-3-[5-(hydroxyacetyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
2-Hydroxy-N-[1-(2-hydroxy-3-{3-[3-({2-[(3S)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
2-Hydroxy-N-(1-{2-hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
2-Hydroxy-N-[1-(2-hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
Ethyl(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
2-Hydroxy-N-[1-(2-hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
N-[1-(2-Hydroxy-3-{5-(hydroxyacetyl)-3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
(2S)-1-(1,5-Dioxa-9-azaspiro[5.5]undec-9-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[4-(2-Methylphenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{4-[2-(Methyloxy)phenyl]piperidin-1-yl}-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(phenylmethyl)piperidin-1-yl]propan-2-ol;
N-{1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
1-(2-{[5-{1-[2-Hydroxy-3-(4-phenylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-dimethylethyl)piperidin-1-yl]propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
(3S)-1-({[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}acetyl)pyrrolidin-3-ol;
N-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-(2-{[5-{1-[2-Hydroxy-3-(4-phenylpiperazin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-ol;
(3R)-1-({[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}acetyl)pyrrolidin-3-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyridin-4-ylpiperidin-1-yl)propan-2-ol;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)ethanesulfonamide;
1-(3-{3-[3-{[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyridin-2-yloxy)piperidin-1-yl]propan-2-ol;
2-(4-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)phenol;
1-(1-{3-[3-{3-[(2-{[2-(1H-imidazol-4-yl)ethyl]amino}ethyl)sulfanyl]-4-(trifluoromethyl)phenyl}-5-(methylsulfonyl)-4,5,6,7-tetrahydro-1H-pyrazolo[4,3-c]pyridin-1-yl]propyl}piperidin-4-yl)pyrrolidin-2-one;
N-{1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-[4-(pyrrolidin-1-ylcarbonyl)piperidin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-phenylpiperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
N-[1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)piperidin-4-yl]methanesulfonamide;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-pyridin-2-ylpiperidin-4-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)-4-pyridin-2-ylpiperidin-4-ol;
N-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyridin-2-yloxy)piperidin-1-yl]propan-2-ol;
1-[4-(2-Fluorophenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(2-pyridin-3-ylpiperidin-1-yl)propan-2-ol;
2-(4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperazin-1-yl)phenol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
1-[1-(3-{3-[3-{[2-(3,3-Dimethylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-[4-(pyrimidin-2-yloxy)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyrimidin-2-yloxy)piperidin-1-yl]propan-2-ol;
(2S)-1-(4-Cyclohexylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[4-(2-Fluorophenyl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(3-Chloropyridin-2-yl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-Cyclopentyl-2-({5-[1-{2-hydroxy-3-[4-(5-oxo-4,5-dihydro-1H-1,2,4-triazol-3-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1,1-Dimethylethyl(2-{3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-oxoethyl)carbamate;
1-[4-(3-Methyl-1,2,4-oxadiazol-5-yl)piperidin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
2-(4-{2-Hydroxy-3-[5-(hydroxyacetyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
4-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)-3-methylpiperazin-2-one;
5-(1-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
N-Cyclopentyl-2-{[5-{1-[2-hydroxy-3-(4-morpholin-4-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}acetamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
1,1-Dimethylethyl 4-{[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]amino}piperidine-1-carboxylate;
(2S)-1-(4-Cyclohexylpiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
5-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-2,4-dihydro-3H-1,2,4-triazol-3-one;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrazin-2-ylpiperazin-1-yl)propan-2-ol;
1-{3-[3-({2-[2-(Hydroxymethyl)morpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
(2S)-2-Hydroxy-N-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}propanamide;
5-Chloro-3-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-1-methyl-1,3-dihydro-2H-benzimidazol-2-one;
N-[4-(Dimethylamino)phenyl]-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
(4R)-1-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-4-hydroxypyrrolidin-2-one;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-phenylpiperidine-4-carbonitrile;
(4S)-1-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-4-hydroxypyrrolidin-2-one;
1-[1-(3-{5-(Methylsulfonyl)-3-[3-({2-[(2-phenylethyl)amino]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
N-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]pyrrolidin-2-one;
1-{4-[2-(Methyloxy)phenyl]piperidin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]ethanesulfonamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
Phenylmethyl(2-{[5-{5-acetyl-1-[2-hydroxy-3-(4-pyridin-2-ylpiperidin-1-yl)propyl]-4,5,6,7-tetrahydro-1H-pyrazolo[4,3-c]pyridin-3-yl}-2-(trifluoromethyl)phenyl]sulfanyl}ethyl)carbamate;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
1-{4-[2-(Methyloxy)phenyl]piperazin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}-4-phenylpiperidin-4-ol;
1-{4-[2-(Methyloxy)phenyl]piperidin-1-yl}-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
N-{1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
N-[1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
N-(1-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
(3R)-1-[({5-[1-{2-Hydroxy-3-[4-(2-hydroxyphenyl)piperazin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-{4-[2-(Methyloxy)phenyl]piperazin-1-yl}-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
1-[4-(2-Chlorophenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
(3R)-1-[({5-[1-{3-[4-(4-Fluorophenyl)piperazin-1-yl]-2-hydroxypropyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-[4-(2-Fluorophenyl)piperazin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
2-[4-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]phenol;
1-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-pyridin-2-ylpiperidin-4-ol;
3-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}phenol;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]methanesulfonamide;
2-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}phenol;
N-{1-[(2S)-2-Hydroxy-3-{5-(methylsulfonyl)-3-[3-{2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}acetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(piperidin-1-ylcarbonyl)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrimidin-2-yloxy)piperidin-1-yl]propan-2-ol;
1-[4-(3-Chloropyridin-2-yl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N-[2-({5-[1-{3-[4-(2-Cyanophenyl)piperazin-1-yl]-2-hydroxypropyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(1H-pyrrolo[2,3-b]pyridin-3-yl)piperidin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
(2S)-1-[4-(1-Hydroxy-1-methylethyl)piperidin-1-yl]-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propan-2-ol;
5-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-2,4-dihydro-3H-1,2,4-triazol-3-one;
(3R)-1-[({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-[5-(Methylsulfonyl)-3-{3-[(2-morpholin-4-ylethyl)sulfanyl]-4-(trifluoromethyl)phenyl]-4,5,6,7-tetrahydro-1H-pyrazolo[4,3-c]pyridin-1-yl]-3-[4-(piperidin-1-ylcarbonyl)piperidin-1-yl]propan-2-ol;
(2R)-1-[4-(2-Fluorophenyl)piperazin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol;
(4R)-4-Hydroxy-1-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
(4S)-4-Hydroxy-1-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[4-(3-Chloropyridin-2-yl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
4-Hydroxy-1-(1-{2-hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[1-(3-{3-[3-{[2-(2,3-Dihydro-1H-inden-2-ylamino)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
2-Hydroxy-N-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}-2-methylpropanamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-morpholin-4-ylpiperidin-1-yl)propan-2-ol;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrimidin-2-yloxy)piperidin-1-yl]propan-2-ol;
2-(4-{2-Hydroxy-3-[5-(methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
4-[1-(2-Hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]benzonitrile;
1-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-4-hydroxypyrrolidin-2-one;
2-(4-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
2-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}benzonitrile;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
2-[4-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
1-{4-[2-(Ethyloxy)phenyl]piperazin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N′-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]-N,N-dimethylsulfamide;
N-[1-(2-Hydroxy-3-{3-[3-{[2-(2-methyl-3-oxopiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-{4-[2-(Methyloxy)phenyl]piperidin-1-yl}-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-[2-({5-[1-(2-Hydroxy-3-{4-[2-(methyloxy)phenyl]piperidin-1-yl}propyl)-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol;
N-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
4-(2-{[5-{1-[2-Hydroxy-3-(4-hydroxy-4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)-3-methylpiperazin-2-one;
1,1-Dimethylethyl[1-({3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}carbonyl)cyclopropyl]carbamate;
2-[4-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperazin-1-yl]phenol;
1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
2-[4-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-{4-[2-(methyloxy)phenyl]piperidin-1-yl}propan-2-ol;
N-{1-[2-({5-[5-(Methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl]sulfanyl)ethyl}piperidin-4-yl}acetamide;
N-[1-(2-Hydroxy-3-{3-[3-({2-[2-(hydroxymethyl)morpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-(2-Hydroxy-3-{3-[3-(2-[2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
1-[1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]pyrrolidin-2-one;
N-Cyclopentyl-2-({5-[1-{2-hydroxy-3-[4-(morpholin-4-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetamide;
4-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]-3-methylpiperazin-2-one;
1-[4-(3-Chloropyridin-2-yl)piperazin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-morpholin-4-ylpiperidin-1-yl)propan-2-ol;
1-{3-[3-({2-[2-(Hydroxymethyl)morpholin-4-yl]ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-{4-[(2-oxo-2-pyrrolidin-1-ylethyl)oxy]piperidin-1-yl}propan-2-ol;
(2R)-1-[4-(3-Chloropyridin-2-yl)piperazin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[1-(3-{3-[3-({2-[(1-Methyl-3-phenylpropyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(morpholin-4-ylcarbonyl)piperidin-1-yl]propan-2-ol;
2-[4-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperazin-1-yl]benzonitrile;
2,2,2-Trifluoro-1-{2-[3-{3-[2-(4-fluoro-piperidin-1-yl)-ethylsulfanyl]-4-trifluoromethyl-phenyl}-1-(2-hydroxy-3-piperidin-1-yl-propyl)-1,4,6,7-tetrahydro-pyrazolo[4,3-c]pyridine-5-carbonyl]-pyrrolidin-1-yl}-ethanone;
N-[1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]methanesulfonamide;
1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
2-[4-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]phenol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
(2S)-2-Hydroxy-N-{1-[(2S)-2-hydroxy-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}propanamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(piperidin-1-ylcarbonyl)piperidin-1-yl]propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(morpholin-4-ylcarbonyl)piperidin-1-yl]propan-2-ol;
1-[1-(3-{5-(Methylsulfonyl)-3-[3-{[2-(3-phenylpiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
2-[4-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
1-[4-(3,5-Dichloropyridin-4-yl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1,1,1-Trifluoro-N-[2-({5-[1-{2-hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[4-(3,5-Dichloropyridin-4-yl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1,1-Dimethylethyl 4-{2-[1-(2-hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl]-2-oxoethyl}piperidine-1-carboxylate;
1-[1-(3-{3-[3-{[2-(1,4′-Bipiperidin-1′-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
Methyl{1-[2-hydroxy-3-(4-pyridin-2-ylpiperidin-1-yl)propyl]-3-[3-{[2-({[(phenylmethyl)oxy]carbonyl}amino)ethyl]sulfanyl}-4-(trifluoromethyl)phenyl]-1,4,6,7-tetrahydro-5H-pyrazolo[4,3-c]pyridin-5-yl}(oxo)acetate;
1,1-Dimethylethyl 3-({3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}carbonyl)piperidine-1-carboxylate;
(4S)-4-Hydroxy-1-{1-[(2S)-2-hydroxy-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}pyrrolidin-2-one;
Ethyl {1-[2-({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)ethyl]piperidin-4-yl}carbamate;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]benzenesulfonamide;
1-[1-(3-{5-(Methylsulfonyl)-3-[3-({2-[4-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-({2-[2-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-({2-[3-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-{4-[1-(phenylmethyl)-1H-tetrazol-5-yl]piperidin-1-yl}propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-{4-[1-(phenylmethyl)-1H-tetrazol-5-yl]piperidin-1-yl}propan-2-ol;
1-(2-Hydroxy-3-{5-(methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}propyl)-4-phenylpiperidin-4-ol;
1-(2-Hydroxy-3-{5-(methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}propyl)-4-pyridin-2-ylpiperidin-4-ol;
4-(1-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2H-1,4-benzoxazin-3(4H)-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
2-{1-[2-Hydroxy-3-(4-pyridin-2-ylpiperidin-1-yl)propyl]-3-[3-{[2-({[(phenylmethyl)oxy]carbonyl}amino)ethyl]sulfanyl}-4-(trifluoromethyl)phenyl]-1,4,6,7-tetrahydro-5H-pyrazolo[4,3-c]pyridin-5-yl}-2-oxoethyl acetate;
1,1-Dimethylethyl 4-(2-{3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-oxoethyl)piperidine-1-carboxylate;
5-Chloro-1-(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-benzimidazol-2-one;
Ethyl {1-[({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)acetyl]piperidin-4-yl}carbamate;
5-Chloro-1-methyl-3-(1-{3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-benzimidazol-2-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-[4-(piperidin-1-ylcarbonyl)piperidin-1-yl]propan-2-ol;
1,1-Dimethylethyl {1-[2-({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)ethyl]piperidin-4-yl}carbamate;
1,1-Dimethylethyl {1-[({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)acetyl]piperidin-4-yl}carbamate;
5-(Dimethylamino)-3-[1-(2-hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
5-(Dimethylamino)-3-[1-(2-hydroxy-3-{3-[3-({2-[(3S)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
1,1-Dimethylethyl 4-{2-[1-(2-hydroxy-3-{4-[(hydroxyacetyl)amino]piperidin-1-yl}propyl)-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl]-2-oxoethyl}piperidine-1-carboxylate; and
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-{4-[1-(phenylmethyl)-1H-tetrazol-5-yl]piperidin-1-yl}propan-2-ol;
and pharmaceutically acceptable salts thereof.

58. A compound as defined in claim 1, wherein said compound is a compound of Formula (I) or a pharmaceutically acceptable salt of a compound of Formula (I).

59. A pharmaceutical composition for treating a disease, disorder, or medical condition mediated by cathepsin S activity, comprising:
(a) an effective amount of at least one chemical entity selected from compounds of Formula (I): 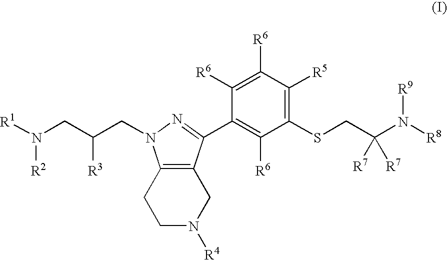
wherein:
R¹ and R² taken together with the nitrogen to which they are attached form a monocyclic heterocycloalkyl group, each group optionally containing one heteroatom ring member in addition to the nitrogen that is O, S, —N═, or NR^{a}, and each group unsubstituted or substituted with one, two, or three R^{b} substituents;
where R^{a} is H, C₁₋₄alkyl, —COC₁₋₄alkyl, or —CO₂C₁₋₄alkyl, or a phenyl ring, monocyclic cycloalkyl ring, or monocyclic heteroaryl ring, each ring unsubstituted or substituted with OH, C₁₋₄alkyl, CF₃, halo, —OC₁₋₄alkyl, cyano, or —COC₁₋₄alkyl; and
each R^{b} substituent is independently:
i) OH, C₁₋₄alkyl, CF₃, —NR^{c}R^{d}, halo, —OC₁₋₄alkyl, CH₂F, CHF₂, —C₁₋₄alkyl-OH, —CH₂NR^{c}R^{d}, —COC₁₋₄alkyl, —CO₂C₁₋₄alkyl, —CO₂H, —CONR^{m}Rⁿ, CN, or —OCH₂CONR^{m}Rⁿ;
ii) a monocyclic heterocycloalkyl group unsubstituted or substituted with C₁₋₄alkyl, OH, —OC₁₋₄alkyl, —NR^{c}R^{d}, or halo;
iii) a monocyclic heterocycloalkyl group fused with a phenyl or pyridyl group, the resulting fused bicyclic group being unsubstituted or substituted with C₁₋₄alkyl, OH, —OC₁₋₄alkyl, —NR^{c}R^{d}, or halo; or
iv) a phenyl group, benzyl group, monocyclic heteroaryl group, monocyclic cycloalkyl group, —O-phenyl group, or —O-(monocyclic heteroaryl) group, each group unsubstituted or substituted with C₁₋₄alkyl, OH, —OC₁₋₄alkyl, CF₃, cyano, —NR^{c}R^{d}, benzyl or halo; or
v) two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, —NR^{c}R^{d}, or halo; or
vi) two R^{b} substituents on adjacent carbons taken together with the carbons to which they are attached form a saturated monocyclic cycloalkyl, phenyl, or monocyclic heteroaryl group, each group unsubstituted or substituted with C₁₋₄alkyl, OH, —OC₁₋₄alkyl, —NR^{c}R^{d}, or halo;
where R^{c} and R^{d} are each independently H, C₁₋₄alkyl, —COC₁₋₄alkyl, —COC₁₋₄alkyl-OH, —CO₂C₁₋₄alkyl, —CONR^{x}R^{y}, —C₁₋₄alkyl-OH, or —SO₂C₁₋₄alkyl; where R^{x} and R^{y} are each independently H or C₁₋₄alkyl; and
R^{m} and Rⁿ are each independently H or C₁₋₄alkyl; or R^{m} and Rⁿ taken together with the nitrogen to which they are attached form a monocyclic heterocycloalkyl ring;
R³ is H, OH, C₁₋₄alkyl, or —OC₁₋₄alkyl;
R⁴ is H; C₁₋₄alkyl; —COC₁₋₄alkyl unsubstituted or substituted with OH, F, —OCOC₁₋₄alkyl, —OC₁₋₄alkyl, phenyl, —SC₁₋₄alkyl, —NR^{o}R^{p}, or —N(R^{o})CO₂C₁₋₄alkyl; —CO-(monocyclic cycloalkyl); —CO-(monocyclic heteroaryl); —CO—(C-linked monocyclic heterocycloalkyl); —COCH₂— (monocyclic heterocycloalkyl); —SO₂C₁₋₄alkyl; —SO₂CF₃; —SO₂—(monocyclic cycloalkyl); —SO₂-phenyl; —SO₂-benzyl; —SO₂—NR^{o}R^{p}; —CONR^{o}R^{p}; —COCO₂C₁₋₄alkyl; —COCONH₂; or —COCONHC₁₋₄alkyl; wherein each cycloalkyl, heteroaryl, heterocycloalkyl, phenyl, or benzyl group is unsubstituted or substituted with C₁₋₄alkyl, OH, F, —COC₁₋₄alkyl, —COCF₃, —CO₂C₁₋₄alkyl, —N(R^{o})CO₂C₁₋₄alkyl, or —NR^{o}R^{p};
where R^{o} and R^{p} are each independently H or C₁₋₄alkyl, or a monocyclic cycloalkyl, phenyl, or monocyclic heteroaryl group; or R^{o} and R^{p} taken together with the nitrogen to which they are attached form a monocyclic heterocycloalkyl group; wherein each cycloalkyl, phenyl, heteroaryl, or heterocycloalkyl group is unsubstituted or substituted with C₁₋₄alkyl, OH, —OC₁₋₄alkyl, —NR^{s}R^{t}, or halo;
where R^{s} and R^{t} are each independently H, C₁₋₄alkyl, —COC₁₋₄alkyl, —CO₂C₁₋₄alkyl, —CONR^{v}R^{w}, C₁₋₄alkyl-OH, or —SO₂C₁₋₄alkyl;
where R^{v} and R^{w} are each independently H or C₁₋₄alkyl;
R⁵ is halo or CF₃;
each R⁶ is independently H or F;
each R⁷ is H or both R⁷ together form a carbonyl;
R⁸ is H or C₁₋₄alkyl and R⁹ is R¹⁰, —COR¹¹, —SO₂R¹¹, —SO₂CF₃, —SO₂N(C₁₋₄alkyl)₂, —CO₂C₁₋₄alkyl, or —CO₂-benzyl;
where R¹⁰ and R¹¹ are each: a C₁₋₄alkyl group unsubstituted or substituted with R^{e}; or a monocyclic cycloalkyl group, bicyclic cycloalkyl group, monocyclic heterocycloalkyl group, phenyl group, or fluorenyl group, each group unsubstituted or substituted with C₁₋₄alkyl, OH, —OC₁₋₄alkyl, —NR^{f}R^{g}, halo, —COC₁₋₄alkyl, —CO₂C₁₋₄alkyl, phenyl, benzyl, or benzyloxy;
where R^{e} is OH, —OC₁₋₄alkyl, or NR^{f}R^{g}, or a monocyclic cycloalkyl group, monocyclic heterocycloalkyl group, monocyclic heteroaryl group, phenyl group, or —O-phenyl group, each group unsubstituted or substituted with C₁₋₄alkyl, OH, —OC₁₋₄alkyl, —NR^{f}R^{g}, halo, —COC₁₋₄alkyl, or —CO₂C₁₋₄alkyl;
where R^{f} and R^{g} are each independently H, C₁₋₄alkyl, —COC₁₋₄alkyl, or —CO₂C₁₋₄alkyl;
or R⁸ and R⁹ taken together with the nitrogen to which they are attached form a monocyclic heterocycloalkyl group, each group optionally containing one heteroatom ring member in addition to the nitrogen that is O, S, SO₂, —N═, or NR^{h}, and each group unsubstituted or substituted with one or two Rⁱ substituents;
where R^{h} is H, C₁₋₄alkyl, phenyl, —C₁₋₂alkyl-phenyl, —COC₁₋₄alkyl, or —CO₂C₁₋₄alkyl; and
each Rⁱ substituent is independently: a C₁₋₄alkyl group unsubstituted or substituted with OH or phenyl; CF₃; halo; OH; —OC₁₋₄alkyl; —NR^{j}R^{k}; —COC₁₋₄alkyl; —CO₂C₁₋₄alkyl; —CONR^{j}R^{k}; phenyl; pyridyl; or benzyl; or two adjacent Rⁱ substituents taken together with the carbons to which they are attached form a phenyl or monocyclic cycloalkyl group;
where R^{j} and R^{k} are each independently H, C₁₋₄alkyl, —COC₁₋₄alkyl, —COC₁₋₄alkyl-OH, or —CO₂C₁₋₄alkyl; or R^{j} and R^{k} taken together with the nitrogen to which they are attached form a monocyclic saturated heterocycloalkyl ring;
and pharmaceutically acceptable salts, prodrugs, and metabolites thereof; and
(b) a pharmaceutically acceptable excipient.

60. A pharmaceutical composition according to claim 59, wherein said chemical entity is selected from the group consisting of:
1-{1-[3-(3-{3-[2-(4-Hydroxy-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(octahydro-isoquinolin-2-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4,4-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(3-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3,5-Dimethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(octahydro-quinolin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,6-Dihydro-2H-pyridin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(1,1-Dioxo-1λ⁶-thiomorpholin-4-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(4-tert-Butyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Fluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
(S)-1-{1-[3-(5-Methanesulfonyl-3-{4-trifluoromethyl-3-[2-(2-trifluoromethyl-pyrrolidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{5-Methanesulfonyl-3-[3-(2-morpholin-4-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{3-[3-(2-Cyclopentylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-(1-{3-[3-(3-{2-[4-(2-Hydroxy-ethyl)-piperidin-1-yl]-ethylsulfanyl}-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one;
1-[1-(3-{5-Methanesulfonyl-3-[3-(2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-phenyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-[3-[2-(Cyclohexyl-methyl-amino)-ethylsulfanyl]-4-trifluoromethyl-phenyl]-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-pyrrolidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-[1-(3-{3-[3-(2-Benzylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-methylamino-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(3-methoxy-propylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-[1-(3-{3-[3-(2-Dimethylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{3-[2-(4-Dimethylamino-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl}-piperidine-4-carboxylic acid amide;
1-{1-[3-(5-Methanesulfonyl-3-{3-[2-(4-methoxy-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{4-trifluoromethyl-3-[2-(3-trifluoromethyl-piperidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(2-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{4-trifluoromethyl-3-[2-(4-trifluoromethyl-piperidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-[1-(3-{5-Methanesulfonyl-3-[3-(2-thiazolidin-3-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-(1-{3-[5-Methanesulfonyl-3-(3-{2-[(thiophen-2-ylmethyl)-amino]-ethylsulfanyl}-4-trifluoromethyl-phenyl)-4,5,6,7-tetrahydro-pyrazolo[4,3-c]pyridin-1-yl]-propyl}-piperidin-4-yl)-pyrrolidin-2-one;
1-{1-[3-(3-{3-[2-(3-Hydroxy-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,4-Dihydro-1H-isoquinolin-2-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-[1,4]diazepan-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(2-Cyclohex-1-enyl-ethylamino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-phenoxy-ethylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-morpholin-4-yl-ethylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-phenethyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-(1-{3-[3-(3-{2-[(1-Ethyl-pyrrolidin-2-ylmethyl)-amino]-ethylsulfanyl}-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one;
1-{1-[3-(3-{3-[2-(Benzyl-methyl-amino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl}-piperidine-3-carboxylic acid diethylamide;
1-{1-[3-(3-{3-[2-(3-Dimethylamino-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
5-Methanesulfonyl-3-[3-(2-piperidin-1-yl-ethylsulfanyl)-4-trifluoromethyl-phenyl]-1-(3-piperidin-1-yl-propyl)-4,5,6,7-tetrahydro-1H-pyrazolo[4,3-c]pyridine;
5-Methanesulfonyl-3-[3-(2-piperidin-1-yl-ethylsulfanyl)-4-trifluoromethyl-phenyl]-1-[3-(4-pyrrolidin-1-yl-piperidin-1-yl)-propyl]-4,5,6,7-tetrahydro-1H-pyrazolo[4,3-c]pyridine;
1-[4-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperazin-1-yl]-ethanone;
5-Methanesulfonyl-3-[3-(2-piperidin-1-yl-ethylsulfanyl)-4-trifluoromethyl-phenyl]-1-(3-pyrrolidin-1-yl-propyl)-4,5,6,7-tetrahydro-1H-pyrazolo[4,3-c]pyridine;
1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-pyrrolidin-3-ol;
5-Methanesulfonyl-1-(3-morpholin-4-yl-propyl)-3-[3-(2-piperidin-1-yl-ethylsulfanyl)-4-trifluoromethyl-phenyl]-4,5,6,7-tetrahydro-1H-pyrazolo[4,3-c]pyridine;
4-[1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-4H-benzo[1,4]oxazin-3-one;
{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-carbamic acid tert-butyl ester;
1-[1-(2-Hydroxy-3-{5-methanesulfonyl-3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
N-Biphenyl-4-yl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-(2-phenoxy-ethyl)-acetamide;
1-{1-[2-Hydroxy-3-(5-methanesulfonyl-3-{3-[2-oxo-2-(4-phenyl-3,6-dihydro-2H-pyridin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-indan-2-yl-acetamide;
N-Biphenyl-3-yl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-(9H-Fluoren-1-yl)-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-(4-Benzyloxy-phenyl)-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-Benzyl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-phenethyl-acetamide;
1-{1-[3-(3-{3-[2-(3-Hydroxy-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-pyrrolidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxymethyl-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxy-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(3-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(octahydro-isoquinolin-2-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Fluoro-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
(S)-1-{1-[3-(5-Methanesulfonyl-3-{3-[2-oxo-2-(2-trifluoromethyl-pyrrolidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
N-(4-Fluoro-phenyl)-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetamide;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-N,N-dimethyl-acetamide;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-N-methyl-acetamide;
1-{1-[3-(3-{3-[2-(4-Dimethylamino-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Dimethylamino-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[3-(2-[1,4′]Bipiperidinyl-1′-yl-2-oxo-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetyl}-piperidine-4-carboxylic acid amide;
1-[1-(3-{5-Methanesulfonyl-3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-oxo-2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-morpholin-4-yl-2-oxo-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{3-[3-(2-Azepan-1-yl-2-oxo-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-methyl-[1,4]diazepan-1-yl)-2-oxo-ethylsulfanyl]-4-trifluoromethyl-phenyl}-4,5,6,7-tetrahydro-pyrazolo[4,3c]pyridin-1-yl)-propyl]-piperidin-4-yl}-pyrrolidin-2-one;
1-{1-[3-(3-{3-[2-(3,6-Dihydro-2H-pyridin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,4-Dihydro-1H-isoquinolin-2-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethylphenylsulfanyl]-N-methyl-N-(1-methyl-piperidin-4-yl)-acetamide;
1-{1-[3-(5-Methanesulfonyl-3-{3-[2-oxo-2-(4-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
N-Cyclohexyl-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethylphenylsulfanyl]-N-methyl-acetamide;
N-Benzyl-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetamide;
1-{1-[3-(5-Methanesulfonyl-3-{3-[2-(4-methyl-piperazin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Benzyl-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-oxo-2-(4-phenethyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-oxo-2-(4-phenyl-3,6-dihydro-2H-pyridin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl phenylsulfanyl]-acetyl}-piperidine-4-carboxylic acid diethylamide;
(R)-1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetyl}-pyrrolidine-2-carboxylic acid amide;
1-{1-[3-(3-{3-[2-(4-Isopropyl-piperazin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
2-(5-{5-Methanesulfonyl-1-[3-(4-pyrrolidin-1-yl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone;
2-(5-{5-Methanesulfonyl-1-[3-(4-trifluoromethyl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone;
2-(5-{1-[3-(4-Acetyl-piperazin-1-yl)-propyl]-5-methanesulfonyl-4,5,6,7-tetrahydro-1H-pyrazolo[4,3-c]pyridin-3-yl}-2-trifluoromethyl-phenylsulfanyl)-1-morpholin-4-yl-ethanone;
-(3-[5-Methanesulfonyl-3-{3-(2-morpholin-4-yl-2-oxo-ethylsulfanyl)-4-trifluoromethyl-phenyl]-4,5,6,7-tetrahydro-pyrazolo[4,3-c]pyridin-1-yl}-propyl)-piperidin-4-yl]-carbamic acid tert-butyl ester;
N-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
N-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-2-phenyl-acetamide;
4-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-piperidine-1-carboxylic acid tert-butyl ester;
({2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-methyl)-carbamic acid tert-butyl ester;
4-Fluoro-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
4-Hydroxy-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
4-Dimethylamino-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
2-(4-Fluoro-phenyl)-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-acetamide;
3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-piperidine-1-carboxylic acid tert-butyl ester;
3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-pyrrolidine-1-carboxylic acid tert-butyl ester;
(3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-propyl)-carbamic acid tert-butyl ester;
Piperidine-3-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
Pyrrolidine-3-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
Piperidine-4-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
(2S)-1-(4,4-Dimethyl-piperidin-1-yl)-3-(3-{3-[2-(4-fluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propan-2-ol;
(2S)—N-{1-[3-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-acetamide; and
(2S)-1-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-3-(4-methylpiperidin-1-yl)-propan-2-ol;
and pharmaceutically acceptable salts thereof.

61. A pharmaceutical composition according to claim 59, wherein said chemical entity is selected from the group consisting of: 1-[1-(3-{3-[3-(2-Amino-ethylsulfanyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-pyrrolidin-2-one;
1-[1-(3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
N-[1-(3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-acetamide;
5-Dimethylamino-3-[1-(3-{3-[3-(2-hydroxy-ethylsulfanyl)-4-trifluoromethyl-phenyl]-5-methanesulfonyl-4,5,6,7-tetrahydro-pyrazolo[4,3-c]pyridin-1-yl}-propyl)piperidin-4-yl]-1-methyl-1,3-dihydro-imidazo[4,5-b]pyridin-2-one;
[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid ethyl ester;
[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid;
3-(3-Benzylsulfanyl-4-chloro-phenyl)-5-methanesulfonyl-1-(3-morpholin-4-yl-propyl)-4,5,6,7-tetrahydro-1H-pyrazolo[4,3-c]pyridine;
3-(4-Chloro-3-phenethylsulfanyl-phenyl)-5-methanesulfonyl-1-(3-morpholin-4-yl-propyl)-4,5,6,7-tetrahydro-1H-pyrazolo[4,3-c]pyridine;
5-Methanesulfonyl-1-(3-morpholin-4-yl-propyl)-3-[3-(2-phenoxy-ethylsulfanyl)-4-trifluoromethyl-phenyl]-4,5,6,7-tetrahydro-1H-pyrazolo[4,3-c]pyridine;
3-[4-Chloro-3-(furan-2-ylmethylsulfanyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-propylsulfanyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(2-phenoxy-ethylsulfanyl)-phenyl]-5-methanesulfonyl-1-(3-morpholin-4-yl-propyl)-4,5,6,7-tetrahydro-1H-pyrazolo[4,3-c]pyridine;
1-[1-(3-{3-[3-(4-Chloro-benzylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
3-[4-Chloro-3-(4-chloro-benzylsulfanyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-fluoro-benzylsulfanyl)-phenyl]-5-methanesulfonyl-1-(3-morpholin-4-yl-propyl)-4,5,6,7-tetrahydro-1H-pyrazolo[4,3-c]pyridine;
1-[1-(3-{5-Methanesulfonyl-3-[3-(2-phenoxy-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
3-[4-Chloro-3-(2-pyrazin-2-yl-ethylsulfanyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-chloro-propylsulfanyl)-phenyl]-5-methanesulfonyl-1-(3-morpholin-4-yl-propyl)-4,5,6,7-tetrahydro-1H-pyrazolo[4,3-c]pyridine; and
3-(4-Chloro-3-phenylmethanesulfinyl-phenyl)-5-methanesulfonyl-1-(3-morpholin-4-yl-propyl)-4,5,6,7-tetrahydro-1H-pyrazolo[4,3-c]pyridine;
and pharmaceutically acceptable salts thereof.

62. A pharmaceutical composition according to claim 59, wherein said chemical entity is selected from the group consisting of:
1-[1-(3-{3-[3-({2-[(1,1-Dimethylethyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
3-[3-({2-[(3S)-3-Methylmorpholin-4-yl]ethyl]sulfanyl)-4-(trifluoromethyl)phenyl]-5-(methylsulfonyl)-1-(3-piperidin-1-ylpropyl)-4,5,6,7-tetrahydro-1H-pyrazolo[4,3-c]pyridine;
3-[3-({2-[(2R,6R)-2,6-Dimethylmorpholin-4-yl]ethyl]sulfanyl)-4-(trifluoromethyl)phenyl]-5-(methylsulfonyl)-1-(3-piperidin-1-ylpropyl)-4,5,6,7-tetrahydro-1H-pyrazolo[4,3-c]pyridine;
{4-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]morpholin-2-yl]methanol;
{(3S)-4-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]morpholin-3-yl]methanol;
5-(Dimethylamino)-1-methyl-3-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-imidazo[4,5-b]pyridin-2-one;
Ethyl(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
N-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]propan-1-amine;
N-[2-({5-[5-(Methylsulfonyl)-1-{3-[4-(piperidin-1-ylcarbonyl)piperidin-1-yl]propyl}-4,5,6,7-tetrahydro-1H-pyrazolo[4,3-c]pyridin-3-yl]-2-(trifluoromethyl)phenyl}sulfanyl)ethyl]propan-1-amine;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
5-(Dimethylamino)-3-(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1-methyl-1,3-dihydro-2H-imidazo[4,5-b]pyridin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
2-(4-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-(4-Cyclopropylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-morpholin-4-ylpropan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-pyrrolidin-1-ylpropan-2-ol;
1-[4-(Dimethylamino)piperidin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-(4-Methylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-({2-[2-(Hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-[1-(2-Hydroxy-3-{3-[3-({2-[2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
4-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)ethyl]-3-methylpiperazin-2-one;
4-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]-3-methylpiperazin-2-one;
4-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)-3-methylpiperazin-2-one;
1-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)ethyl]piperidin-4-ol;
1-[1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
2-[4-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperazin-1-yl]benzonitrile;
1-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-ol;
5-(Dimethylamino)-3-[1-(2-hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-(4-Methylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4,4-Dimethylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4-Fluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(1,1-Dimethylethyl)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-morpholin-4-ylpropan-2-ol;
5-(Dimethylamino)-3-(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1-methyl-1,3-dihydro-2H-imidazo[4,5-b]pyridin-2-one;
1-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
2-(4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{5-(Ethylsulfonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-3-ol;
1-(2-{[5-{1-[2-Hydroxy-3-(4-hydroxy-4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-3-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(2-hydroxyethyl)piperidin-1-yl]propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxamide;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-2-hydroxyacetamide;
1-{1-[(2S)-2-Hydroxy-3-{3-[3-({2-[(2R)-2-(hydroxymethyl)morpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[(2S)-2-Hydroxy-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]-4-methylpiperidin-4-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(octahydroisoquinolin-2(1H)-yl)propan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrazin-2-ylpiperazin-1-yl)propan-2-ol;
(2R)-1-[4-(3,5-Dichloropyridin-4-yl)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(3,6-Dihydropyridin-1(2H)-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-[4-(1,1-Dimethylethyl)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
N-{1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1,1-dimethylethyl)piperidin-1-yl]propan-2-ol;
(2S)-1-(4-Cyclohexylpiperidin-1-yl)-3-{3-[3-{[2-(4,4-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(Hexahydrocyclopenta[c]pyrrol-2(1H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-methylpiperidin-4-ol;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidine-4-carboxamide;
(2S)-1-{3-[3-({2-[4-(1,1-Dimethylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
N-[1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-yl]acetamide;
2-Hydroxy-N-[1-(2-{[5-{1-[(2S)-2-hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-4-yl]acetamide;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-pyridin-2-ylpiperidin-4-ol;
(2S)-1-{3-[3-({2-[(3S)-3-Methylmorpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(2S)-1-{3-[3-{[2-(Hexahydrocyclopenta[c]pyrrol-2(1H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-methylpiperidin-4-ol;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidine-4-carboxamide;
(2S)-1-{3-[3-({2-[4-(1-Hydroxy-1-methylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
5-(Dimethylamino)-3-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-1-methyl-1,3-dihydro-2H-imidazo[4,5-b]pyridin-2-one;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyrrolidin-1-ylpiperidin-1-yl)propan-2-ol;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-(1-{2-Hydroxy-3-[5-(phenylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-(1-{3-[5-(Ethylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-2-hydroxypropyl}piperidin-4-yl)pyrrolidin-2-one;
1-[5-(Ethylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-{1-[2-Hydroxy-3-(5-[(phenylmethyl)sulfonyl]-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-[5-(Hydroxyacetyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-[5-(Hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-(1-{2-Hydroxy-3-[5-(hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)piperidin-4-yl]pyrrolidin-2-one;
1-[3-{3-[(2-Morpholin-4-ylethyl)sulfanyl]-4-(trifluoromethyl)phenyl}-5-(piperidin-4-ylacetyl)-4,5,6,7-tetrahydro-1H-pyrazolo[4,3-c]pyridin-1-yl]-3-piperidin-1-ylpropan-2-ol;
Methyl[1-(2-hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl](oxo)acetate;
Methyl(1-{2-hydroxy-3-[4-(2-oxopyrrolidin-1-yl)piperidin-1-yl]propyl}-3-{3-[(2-piperidin-1-ylethyl)sulfanyl]-4-(trifluoromethyl)phenyl}-1,4,6,7-tetrahydro-5H-pyrazolo[4,3-c]pyridin-5-yl)(oxo)acetate;
1-(5-Acetyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-{1-[3-(5-Acetyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}pyrrolidin-2-one;
1-(1-{3-[5-(Furan-2-ylcarbonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-2-hydroxypropyl}piperidin-4-yl)pyrrolidin-2-one;
1-(5-Methyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
2-(1-{(2S)-3-[4-(Acetylamino)piperidin-1-yl]-2-hydroxypropyl}-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
2-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-2-oxoacetamide;
(2S)-1-{3-[3-{[2-(4-Fluoropiperidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-piperidin-1-ylpropan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-methyl-1,4,6,7-tetrahydro-5H-pyrazolo[4,3-c]pyridine-5-carboxamide;
5-(Methylsulfonyl)-3-{3-[(2-morpholin-4-yl-2-oxoethyl)sulfanyl]-4-(trifluoromethyl)phenyl}-1-(3-piperidin-1-ylpropyl)-4,5,6,7-tetrahydro-1H-pyrazolo[4,3-c]pyridine;
5-(Methylsulfonyl)-3-{3-[(2-morpholin-4-yl-2-oxoethyl)sulfanyl]-4-(trifluoromethyl)phenyl}-1-(3-morpholin-4-ylpropyl)-4,5,6,7-tetrahydro-1H-pyrazolo[4,3-c]pyridine;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-[5-(Methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-{4-[2-(Methyloxy)phenyl]piperidin-1-yl}-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propan-2-ol;
1-{1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-[4-(1,1-Dimethylethyl)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyridin-2-ylpiperidin-1-yl)propan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-{4-[2-(methyloxy)phenyl]piperidin-1-yl}propan-2-ol;
1-{3-[3-{[2-(4-Fluoropiperidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-(4-pyridin-2-ylpiperidin-1-yl)propan-2-ol;
1-(5-Acetyl-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl)-3-{4-[2-(methyloxy)phenyl]piperidin-1-yl}propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(1,1-dimethylethyl)piperidin-1-yl]propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-methylpiperidin-1-yl)propan-2-ol;
1-{1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}pyrrolidin-2-one; and
1-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
and pharmaceutically acceptable salts thereof.

63. A pharmaceutical composition according to claim 59, wherein said chemical entity is selected from the group consisting of:
1-[3-{3-[2-(4-Fluoro-piperidin-1-yl)-ethylsulfanyl]-4-trifluoromethyl-phenyl}-1-(2-hydroxy-3-piperidin-1-yl-propyl)-1,4,6,7-tetrahydro-pyrazolo[4,3-c]pyridin-5-yl]-2-hydroxy-ethanone;
(2S)-1-(4-Cyclohexyl-piperidin-1-yl)-3-(3-{3-[2-((3S)-fluoro-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propan-2-ol;
N-[1-(2-{5-[1-((2S)-Hydroxy-3-morpholin-4-yl-propyl)-5-methanesulfonyl-4,5,6,7-tetrahydro-1H-pyrazolo[4,3-c]pyridin-3-yl]-2-trifluoromethyl-phenylsulfanyl}-ethyl)-piperidin-4-yl]-acetamide;
(2S)-1-(3-{3-[2-((3S)-Fluoro-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-3-(3′,4′,5′,6′-tetrahydro-2′H-[2,4′]bipyridinyl-1′-yl)-propan-2-ol;
(3S)-1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-3-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-pyrrolidin-1-ylpropan-2-ol;
1-Piperidin-1-yl-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]pyrrolidin-3-ol;
1-(3-{3-[(2-Morpholin-4-ylethyl)sulfanyl]-4-(trifluoromethyl)phenyl-4,5,6,7-tetrahydro-1H-pyrazolo[4,3-c]pyridin-1-yl)-3-piperidin-1-ylpropan-2-ol;
1-Morpholin-4-yl-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(4-Methylpiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-ol;
1-[2-({5-[1-(2-Hydroxy-3-piperidin-1-ylpropyl)-4,5,6,7-tetrahydro-1H-pyrazolo[4,3-c]pyridin-3-yl]-2-(trifluoromethyl)phenyl}sulfanyl)ethyl]piperidin-3-ol;
1-(4-Fluoropiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-morpholin-4-ylpropan-2-ol;
1-(Hexahydrocyclopenta[c]pyrrol-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol
1-(4,4-Dimethylpiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]-4-methylpiperidin-4-ol;
1-[4-(Methyloxy)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(4-Fluoropiperidin-1-yl)ethyl]sulfanyl}-4-(trifluoromethyl)phenyl]-5-methyl-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-methylpiperidin-1-yl)propan-2-ol;
1-(1,3-Dihydro-2H-isoindol-2-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
3-{3-[(2-Azetidin-1-ylethyl)sulfanyl]-4-(trifluoromethyl)phenyl}-5-(methylsulfonyl)-1-(3-piperidin-1-ylpropyl)-4,5,6,7-tetrahydro-1H-pyrazolo[4,3-c]pyridine;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidine-4-carboxamide;
1-(4-Acetylpiperazin-1-yl)-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidine-4-carboxamide;
1-(3,3-Difluoropyrrolidin-1-yl)-3-{3-[3-{[2-(3,3-difluoropyrrolidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-(4,4-Dimethylpiperidin-1-yl)-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
5-(Methylsulfonyl)-1-(3-piperidin-1-ylpropyl)-3-{3-[(2-pyrrolidin-1-ylethyl)sulfanyl]-4-(trifluoromethyl)phenyl}-4,5,6,7-tetrahydro-1H-pyrazolo[4,3-c]pyridine;
1-(3,4-Dihydroisoquinolin-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
N-Methyl-N-[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl}propan-2-amine;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[(3S)-3-(trifluoromethyl)pyrrolidin-1-yl]propan-2-ol;
1-(Octahydroisoquinolin-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
2-(1-[(2S)-2-Hydroxy-3-piperidin-1-ylpropyl]-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
1-(1,4-Dioxa-8-azaspiro[4.5]dec-8-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[4-(1-Hydroxy-1-methylethyl)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}urea;
(2S)-1-{5-Acetyl-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidine-4-carboxamide;
1-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl}imidazolidin-4-one;
1-{3-[3-{[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
5-(Methylsulfonyl)-3-{3-[(2-morpholin-4-ylethyl)sulfanyl]-4-(trifluoromethyl)phenyl}-1-(3-piperidin-1-ylpropyl)-4,5,6,7-tetrahydro-1H-pyrazolo[4,3-c]pyridine;
1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidine-4-carboxamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-pyrrolidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(hexahydrocyclopenta[c]pyrrol-2(1H)-yl)propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4,4-dimethylpiperidin-1-yl)propan-2-ol;
1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidine-4-carboxamide;
2-Hydroxy-N-{1-[2-hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
2-Hydroxy-N-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
(2S)-1-[1-(2-Hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl]-1-oxopropan-2-ol;
1-(5-[(Methyloxy)acetyl]-3-{3-[(2-morpholin-4-ylethyl)sulfanyl]-4-(trifluoromethyl)phenyl]-4,5,6,7-tetrahydro-1H-pyrazolo[4,3-c]pyridin-1-yl)-3-piperidin-1-ylpropan-2-ol;
1-[2-({5-[5-(Hydroxyacetyl)-1-(2-hydroxy-3-piperidin-1-ylpropyl)-4,5,6,7-tetrahydro-1H-pyrazolo[4,3-c]pyridin-3-yl]-2-(trifluoromethyl)phenyl]sulfanyl)ethyl}piperidin-3-ol;
(2S)-1-{3-[3-{[2-(4-Fluoropiperidin-1-yl)ethyl]sulfanyl}-4-(trifluoromethyl)phenyl]-5-propanoyl-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-glycyl-4,5,6,7-tetrahydro-1H-pyrazolo[4,3-c]pyridin-1-yl}-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(1,3-dihydro-2H-isoindol-2-yl)propan-2-ol;
N-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl}cyclohexanamine;
1-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}urea;
N-(2-{[5-{1-[3-(4-Cyclopropylpiperazin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)propan-1-amine;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-4-ylpiperidin-1-yl)propan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
1-(4-Methylpiperidin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(4-Methylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}pyrrolidin-3-ol;
1-[(3S)-3-(Hydroxymethyl)pyrrolidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[(3S)-3-Methylmorpholin-4-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]urea;
(2S)-1-(5,6-Dihydropyrimidin-1(4H)-yl)-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-morpholin-4-ylpropan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}pyrrolidin-3-ol;
5-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-2,4-dihydro-3H-1,2,4-triazol-3-one;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
2-{1-[(2S)-2-Hydroxy-3-piperidin-1-ylpropyl]-3-[3-{[2-(4-methylpiperidin-1-yl)ethyl]sulfanyl}-4-(trifluoromethyl)phenyl]-1,4,6,7-tetrahydro-5H-pyrazolo[4,3-c]pyridin-5-yl}-2-oxoacetamide;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-(4-pyrrolidin-1-ylpiperidin-1-yl)propan-2-ol;
1-(4-Morpholin-4-ylpiperidin-1-yl)-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
2-{1-(2-Hydroxy-3-piperidin-1-ylpropyl)-3-[3-{[2-(4-methylpiperidin-1-yl)ethyl]sulfanyl}-4-(trifluoromethyl)phenyl]-1,4,6,7-tetrahydro-5H-pyrazolo[4,3-c]pyridin-5-yl}-2-oxoacetamide;
N-{1-[(2S)-3-(5-Acetyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}acetamide;
(4R)-1-{1-[(2S)-3-(5-Acetyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}-4-hydroxypyrrolidin-2-one;
Ethyl{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}carbamate;
N-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)ethyl}methanesulfonamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-dimethyl-1,4,6,7-tetrahydro-5H-pyrazolo[4,3-c]pyridine-5-carboxamide;
1-(5-[(Methylsulfanyl)acetyl]-3-{3-[(2-morpholin-4-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(3,4-dihydroisoquinolin-2(1H)-yl)propan-2-ol;
(2S)-1-{3-[3-{[2-(4-Fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-1-oxopropan-2-ol;
(2S)-1-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-1-oxopropan-2-ol;
1-{3-[3-{[2-(4-Fluoropiperidin-1-yl)ethyl]sulfanyl}-4-(trifluoromethyl)phenyl]-5-[(methyloxy)acetyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-[(methyloxy)acetyl]-4,5,6,7-tetrahydro-1H-pyrazolo[4,3-c]pyridin-1-yl}-3-piperidin-1-ylpropan-2-ol;
3-Methyl-4-[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl}piperazin-2-one;
N-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}-2-hydroxyacetamide;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(4,4-Dimethylpiperidin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
2-{4-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
N-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
1-(4-Methyl-1,4-diazepan-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
3-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
4-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
2-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}-4-methylpiperidin-4-ol;
(2S)-1-(3,6-Dihydropyridin-1(2H)-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
(3S)-1-[({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
(3R)-1-[({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(2S)-1-{3-[3-({2-[(3S)-3-Methylmorpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(3S)-1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-3-ol;
(2S)-1-(5,6-Dihydropyrimidin-1(4H)-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(5,6-Dihydropyrimidin-1(4H)-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-(4-Methylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(4-Fluorophenyl)piperazin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
N-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]-2-hydroxyacetamide;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-4-ylpiperidin-1-yl)propan-2-ol;
1-[4-(2-Fluorophenyl)piperazin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
1-[4-(4-Fluorophenyl)piperazin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-Azepan-1-yl-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
(3R)-1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-3-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-ol;
(3S)-1-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}pyrrolidin-3-ol;
(3R)-1-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}pyrrolidin-3-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(3S)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-ol;
N-Methyl-2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)-N-(phenylmethyl)ethanamine;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperidin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-[1-(2-Hydroxy-3-{3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
(4R)-4-Hydroxy-1-{1-[(2S)-2-hydroxy-3-(5-methyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(1-hydroxy-1-methylethyl)piperidin-1-yl]propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(1,4-dioxa-8-azaspiro[4.5]dec-8-yl)propan-2-ol;
(2S)-1-{5-(Cyclobutylcarbonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
4-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
3-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
4-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
1-{5-[(1-Aminocyclopropyl)carbonyl]-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}pyrrolidin-2-one;
N-(2-{[5-{1′-[2-Hydroxy-3-(4-methylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
2-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}benzonitrile;
2-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-N-methyl-2-oxoacetamide;
1-{5-(N,N-Dimethylglycyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-(1-methylethyl)-1,4,6,7-tetrahydro-5H-pyrazolo[4,3-c]pyridine-5-carboxamide;
Methyl {3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}(oxo)acetate;
Methyl {3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}(oxo)acetate;
1-{3-[3-{[2-(4-Fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-methyl-1-oxopropan-2-ol;
1-(4-Cyclopropylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]pyrrolidin-2-one;
N-(2-{[5-{5-(Methylsulfonyl)-1-[3-(4-pyrrolidin-1-ylpiperidin-1-yl)propyl]-4,5,6,7-tetrahydro-1H-pyrazolo[4,3-c]pyridin-3-yl}-2-(trifluoromethyl)phenyl]sulfanyl]ethyl)propan-1-amine;
(2S)-1-{3-[3-{[2-(4-Fluoropiperidin-1-yl)ethyl]sulfanyl}-4-(trifluoromethyl)phenyl]-5-[(methylsulfanyl)acetyl]-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-[(methylsulfanyl)acetyl]-4,5,6,7-tetrahydro-1H-pyrazolo[4,3-c]pyridin-1-yl}-3-piperidin-1-ylpropan-2-ol;
1-{4-[2-(Methyloxy)phenyl]piperidin-1-yl}-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-(4-Methylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-Azepan-1-yl-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{4-[4-(Methyloxy)phenyl]piperidin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[2-(Methyloxy)phenyl]piperidin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
1-(4-Acetylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}pyrrolidin-3-yl)acetamide;
1-{4-[3-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[4-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[2-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}praline;
(2S)-1-[(3S)-3-Methylmorpholin-4-yl]-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(3,3-Dimethylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-3-methylpiperazin-2-one;
N-{1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]-4-pyridin-2-ylpiperidin-4-ol;
1-(2-{[5-{1-[2-Hydroxy-3-(4-hydroxy-4-pyridin-2-ylpiperidin-1-yl)propyl]-4,5,6,7-tetrahydro-1H-pyrazolo[4,3-c]pyridin-3-yl}-2-(trifluoromethyl)phenyl]sulfanyl]ethyl)piperidin-3-ol;
N-[1-(2-Hydroxy-3-{3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
(2S)-1-(4,4-Dimethylpiperidin-1-yl)-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
1-[(3S)-3-(Hydroxymethyl)pyrrolidin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-[(3S)-3-Methylmorpholin-4-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
1-[4-(3-Chlorophenyl)piperidin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[4-(Methyloxy)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-Azepan-1-yl-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-Azepan-1-yl-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
1-(2-{[5-(1-{2-Hydroxy-3-[4-(pyrimidin-2-yloxy)piperidin-1-yl]propyl}-4,5,6,7-tetrahydro-1H-pyrazolo[4,3-c]pyridin-3-yl)-2-(trifluoromethyl)phenyl]sulfanyl]ethyl)piperidin-3-ol;
1-(4-Aminopiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
N-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}methanesulfonamide;
2-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}phenol;
3-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}phenol;
1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-ol;
(3S)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-3-ol;
1-[2-(Hydroxymethyl)morpholin-4-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-3-ol;
1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(3,6-dihydropyridin-1(2H)-yl)propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-3-ol;
(3R)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-3-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-ol;
N-Methyl-2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)-N-(2-phenylethyl)ethanamine;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[(2S)-2-(hydroxymethyl)pyrrolidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(5,6-dihydropyrimidin-1(4H)-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(5,6-dihydropyrimidin-1(4H)-yl)propan-2-ol;
(2S)-1-{3-[3-{[2-(4-Fluoropiperidin-1-yl)ethyl]sulfanyl}-4-(trifluoromethyl)phenyl]-5-(1H-1,2,4-triazol-3-ylcarbonyl)-4,5,6,7-tetrahydro-1H-pyrazolo[4,3-c]pyridin-1-yl}-3-piperidin-1-ylpropan-2-ol;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
1-[4-(Morpholin-4-ylcarbonyl)piperidin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(2-{[5-(1-{2-Hydroxy-3-[4-(pyrrolidin-1-ylcarbonyl)piperidin-1-yl]propyl}-4,5,6,7-tetrahydro-1H-pyrazolo[4,3-c]pyridin-3-yl)-2-(trifluoromethyl)phenyl]sulfanyl]ethyl)piperidin-3-ol;
(2S)-1-[4-(2-Fluorophenyl)piperazin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-[4-(4-Fluorophenyl)piperazin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-(3-Fluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-(4-Fluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(4-Fluoropiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
N-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]methanesulfonamide;
4-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]phenol;
1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)-4-pyridin-2-ylpiperidin-4-ol;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
3-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]phenol;
2-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]pyridine-3-carbonitrile;
(2S)-1-{3-[3-{[2-(4-Fluoropiperidin-1-yl)ethyl]sulfanyl}-4-(trifluoromethyl)phenyl]-5-(pyrrolidin-1-ylcarbonyl)-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
N-(2-{[5-{1-[3-(4,4-Dimethylpiperidin-1-yl)-2-hydroxypropyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-(4,4-Difluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(3,3-Difluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
1-{3-[3-{[2-(4-Fluoropiperidin-1-yl)ethyl]sulfanyl}-4-(trifluoromethyl)phenyl]-5-[(2R)-tetrahydrofuran-2-ylcarbonyl]-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(4-fluorophenyl)piperazin-1-yl]propan-2-ol;
N-(1,1-Dimethylethyl)-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-(1-methylpropyl)-1,4,6,7-tetrahydro-5H-pyrazolo[4,3-c]pyridine-5-carboxamide;
1-[1-(3-{3-[3-{[2-(Cyclopropylamino)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
N,N-Diethyl-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
2-{4-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperazin-1-yl}benzonitrile;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(2,4,6,7-tetrahydro-5H-pyrazolo[4,3-c]pyridin-5-yl)propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(1,4,6,7-tetrahydro-5H-imidazo[4,5-c]pyridin-5-yl)propan-2-ol;
N-(1-{3-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
1-[1-(3-{3-[3-({2-[(1-Methylethyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-{[2-(propylamino)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
4-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}benzonitrile;
1-(4-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenyl)ethanone;
N,N-Dimethyl-1-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-3-yl)methanamine;
3-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}benzonitrile;
2-[4-(3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]benzonitrile;
2-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}benzonitrile;
1-[4-(1,1-Dimethylethyl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-(4,4-Dimethylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{4-[2-(Methyloxy)phenyl]piperidin-1-yl}-3-(5-methyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(4-Cyclopropylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4-Acetylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
2-{4-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperazin-1-yl}phenol;
1-[4-(1-Methylethyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{4-[2-(Ethyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{5-(Cyclopropylsulfonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[4-(1-Hydroxy-1-methylethyl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]-4-methylpiperidin-4-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
N-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}pyrrolidin-3-yl)acetamide;
1-(4-Acetylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(Dimethylamino)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(1-Methylethyl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidine-4-carboxamide;
1-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(4-fluorophenyl)piperazin-1-yl]propan-2-ol;
(2S)-1-{3-[3-{[2-(4-Fluoropiperidin-1-yl)ethyl]sulfanyl}-4-(trifluoromethyl)phenyl]-5-(propylsulfonyl)-4,5,6,7-tetrahydro-1H-pyrazolo[4,3-c]pyridin-1-yl}-3-piperidin-1-ylpropan-2-ol
(2S)-1-{3-[3-{[2-(4-Fluoropiperidin-1-yl)ethyl]sulfanyl}-4-(trifluoromethyl)phenyl]-5-[(1-methylethyl)sulfonyl]-4,5,6,7-tetrahydro-1H-pyrazolo[4,3-c]pyridin-1-yl}-3-piperidin-1-ylpropan-2-ol;
(2S)-1-(4,4-Dimethylpiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
2-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]benzonitrile;
(2R)-1-(4,4-Dimethylpiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-(4,4-Dimethylpiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(methyloxy)phenyl]piperidin-1-yl}propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[4-(methyloxy)phenyl]piperidin-1-yl}propan-2-ol;
1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-dimethyl-1,4,6,7-tetrahydro-5H-pyrazolo[4,3-c]pyridine-5-sulfonamide;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(methyloxy)phenyl]piperazin-1-yl}propan-2-ol;
1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4-Fluoropiperidin-1-yl)ethyl]sulfanyl}-4-(trifluoromethyl)phenyl]-5-[(1-methyl-1H-imidazol-2-yl)carbonyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(hydroxymethyl)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(hydroxymethyl)piperidin-1-yl]propan-2-ol;
1-(5-Acetyl-3-{3-[(2-morpholin-4-yl-2-oxoethyl)sulfanyl]-4-(trifluoromethyl)phenyl-4,5,6,7-tetrahydro-1H-pyrazolo[4,3-c]pyridin-1-yl)-3-(4-pyrrolidin-1-ylpiperidin-1-yl)propan-2-ol;
1-(3,4-Dihydroisoquinolin-2(1H)-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-Azepan-1-yl-3-{3-[3-{[2-(3,3-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-Azepan-1-yl-3-{3-[3-{[2-(4,4-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(2-thienylcarbonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(2-thienylcarbonyl)-4,5,6,7-tetrahydro-1H-pyrazolo[4,3-c]pyridin-1-yl}-3-piperidin-1-ylpropan-2-ol;
(2S)-1-[4-(3-Chlorophenyl)piperidin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
N-[1-(3-{5-(Methylsulfonyl)-3-[3-{[2-(propylamino)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[3-(methyloxy)phenyl]piperazin-1-yl}propan-2-ol;
1-{3-[3-{[2-(4-Fluoropiperidin-1-yl)ethyl]sulfanyl}-4-(trifluoromethyl)phenyl]-5-(1-methyl-L-prolyl)-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[4-(methyloxy)phenyl]piperazin-1-yl}propan-2-ol;
1-{3-[3-{[2-(4-Fluoropiperidin-1-yl)ethyl]sulfanyl}-4-(trifluoromethyl)phenyl]-5-(piperidin-3-ylcarbonyl)-4,5,6,7-tetrahydro-1H-pyrazolo[4,3-c]pyridin-1-yl}-3-piperidin-1-ylpropan-2-ol;
4-{1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}benzonitrile;
2-Hydroxy-N-{1-[2-hydroxy-3-(5-[(methyloxy)acetyl]-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
(2S)-1-{3-[3-{[2-(4-Fluoropiperidin-1-yl)ethyl]sulfanyl}-4-(trifluoromethyl)phenyl]-5-(morpholin-4-ylcarbonyl)-4,5,6,7-tetrahydro-1H-pyrazolo[4,3-c]pyridin-1-yl}-3-piperidin-1-ylpropan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-(4,4-Difluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{5-(Methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[1-(3-{3-[3-({2-[(2-Methylpropyl)amino]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-pyrrolidin-1-ylpropan-2-ol;
(2S)-1-{5-(Methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}-3-morpholin-4-ylpropan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(octahydroisoquinolin-2(1H)-yl)propan-2-ol;
N-[(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methyl]acetamide;
1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidine-4-carboxamide;
1-[1-(3-{3-[3-({2-[(3-Hydroxypropyl)amino]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[5-(Hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)urea;
1-(1,5-Dioxa-9-azaspiro[5.5]undec-9-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-{3-[3-({2-[4-(1-Hydroxy-1-methylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
2-Hydroxy-N-(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-phenyl-1,4,6,7-tetrahydro-5H-pyrazolo[4,3-c]pyridine-5-carboxamide;
(2S)-1-[4-(1,1-Dimethylethyl)piperidin-1-yl]-3-{3-[3-({2-[(3S)-3-fluoropyrrolidin-1-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propan-2-ol;
N-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
(2S)-1-[4-(Dimethylamino)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
(2S)-1-{4-[2-(Ethyloxy)phenyl]piperazin-1-yl}-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxylic acid;
(3S)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-3-carboxylic acid;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-3-carboxylic acid;
(3R)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-3-carboxylic acid;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-phenylpiperidin-1-yl)propan-2-ol;
1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-3-carboxylic acid;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-yl]acetamide;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(2-hydroxyethyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4,4-dimethylpiperidin-1-yl)propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4,4-dimethylpiperidin-1-yl)propan-2-ol;
N-[1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-yl]acetamide;
1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-2-thienyl-1,4,6,7-tetrahydro-5H-pyrazolo[4,3-c]pyridine-5-carboxamide;
1-{3-[3-{[2-(4-Fluoropiperidin-1-yl)ethyl]sulfanyl}-4-(trifluoromethyl)phenyl]-5-(piperidin-4-ylacetyl)-4,5,6,7-tetrahydro-1H-pyrazolo[4,3-c]pyridin-1-yl}-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyrrolidin-1-ylpiperidin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(ethyloxy)phenyl]piperazin-1-yl}propan-2-ol;
N-Cyclohexyl-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4-Fluoropiperidin-1-yl)ethyl]sulfanyl}-4-(trifluoromethyl)phenyl]-5-[(4-methylpiperazin-1-yl)carbonyl]-4,5,6,7-tetrahydro-1H-pyrazolo[4,3-c]pyridin-1-yl}-3-piperidin-1-ylpropan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-{4-[3-(trifluoromethyl)phenyl]piperazin-1-yl}propan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-bis(1-methylethyl)-1,4,6,7-tetrahydro-5H-pyrazolo[4,3-c]pyridine-5-carboxamide;
(2S)-1-[4-(1,1-Difluoroethyl)piperidin-1-yl]-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(4-Methylpiperidin-1-yl)-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propan-2-ol;
2-Hydroxy-N-{1-[2-hydroxy-3-(5-[(methylsulfanyl)acetyl]-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
1-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-(1,4′-Bipiperidin-1′-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(trifluoromethyl)piperidin-1-yl]propan-2-ol;
(2S)-1-[4-(1,1-Dimethylethyl)piperidin-1-yl]-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol; N-{1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}acetamide;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
(2S)-1-{3-[3-{[2-(4-Fluoropiperidin-1-yl)ethyl]sulfanyl}-4-(trifluoromethyl)phenyl]-5-[(trifluoromethyl)sulfonyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(octahydroquinolin-1(2H)-yl)propan-2-ol;
(2S)-1-[4-(1-Hydroxy-1-methylethyl)piperidin-1-yl]-3-{3-[3-{[2-(4-methylpiperidin-1-yl)ethyl]sulfanyl-4-(trifluoromethyl)phenyl]-5-(methylsulfonyl)-4,5,6,7-tetrahydro-1H-pyrazolo[4,3-c]pyridin-1-yl}propan-2-ol;
N-[(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)methyl]acetamide;
Ethyl(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
(2S)-1-[4-(1,1-Dimethylethyl)piperidin-1-yl]-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(4-Fluoropiperidin-1-yl)ethyl]sulfanyl}-4-(trifluoromethyl)phenyl]-5-[(2S)-2-hydroxy-2-phenylacetyl]-4,5,6,7-tetrahydro-1H-pyrazolo[4,3-c]pyridin-1-yl}-3-piperidin-1-ylpropan-2-ol;
2-Hydroxy-N-[1-(2-{[5-{1-[(2S)-2-hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-yl]acetamide;
N-{1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
2-Hydroxy-N-(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide; N-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-phenylpiperazin-1-yl)propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-4-ylpiperidin-1-yl)propan-2-ol;
1-(5-Acetyl-3-{3-[(2-morpholin-4-yl-2-oxoethyl)sulfanyl]-4-(trifluoromethyl)phenyl-4,5,6,7-tetrahydro-1H-pyrazolo[4,3-c]pyridin-1-yl)-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-phenylpiperidin-1-yl)propan-2-ol;
2-(1-[(2S)-2-Hydroxy-3-{4-[(4R)-4-hydroxy-2-oxopyrrolidin-1-yl]piperidin-1-yl}propyl]-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyridin-4-ylpiperidin-1-yl)propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-phenylpiperazin-1-yl)propan-2-ol;
N-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
1-[1-(2-Hydroxy-3-{5-(hydroxyacetyl)-3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
1-{5-(1-Acetyl-L-prolyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyrimidin-2-ylpiperazin-1-yl)propan-2-ol;
1-[1-(3-[3-[3-{[2-(Cyclohexylamino)ethyl]sulfanyl]-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]-4-[3-(trifluoromethyl)phenyl]piperidin-4-ol;
2-(4-{2-Hydroxy-3-[5-(hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
5-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-Hydroxy-1-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[4-(3-Methyl-1,2,4-oxadiazol-5-yl)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-cyclopropylpiperazin-1-yl)propan-2-ol;
5-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-Hydroxy-1-(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one; 1-[5-(Methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[3-(trifluoromethyl)phenyl]piperazin-1-yl}propan-2-ol;
N-(2-{[5-{1′-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
Ethyl(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
N-(1-[2-Hydroxy-3-[5-(hydroxyacetyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
2-Hydroxy-N-[1-(2-hydroxy-3-{3-[3-({2-[(3S)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
2-Hydroxy-N-(1-[2-hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
2-Hydroxy-N-[1-(2-hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
Ethyl(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
2-Hydroxy-N-[1-(2-hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide; N-[1-(2-Hydroxy-3-{5-(hydroxyacetyl)-3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
(2S)-1-(1,5-Dioxa-9-azaspiro[5.5]undec-9-yl)-3-{3-[3-[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[4-(2-Methylphenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{4-[2-(Methyloxy)phenyl]piperidin-1-yl}-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(phenylmethyl)piperidin-1-yl]propan-2-ol;
N-{1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
1-(2-{[5-{1-[2-Hydroxy-3-(4-phenylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1,1-dimethylethyl)piperidin-1-yl]propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
(3S)-1-({[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]acetyl)pyrrolidin-3-ol;
N-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-(2-{[5-{1-[2-Hydroxy-3-(4-phenylpiperazin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
(3R)-1-({[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]acetyl)pyrrolidin-3-ol; 1-[5-(Methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-(4-pyridin-4-ylpiperidin-1-yl)propan-2-ol;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)ethanesulfonamide;
1-(3-{3-[3-[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyridin-2-yloxy)piperidin-1-yl]propan-2-ol;
2-(4-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)phenol;
1-(1-{3-[3-{3-[(2-{[2-(1H-imidazol-4-yl)ethyl]amino]ethyl)sulfanyl]-4-(trifluoromethyl)phenyl}-5-(methylsulfonyl)-4,5,6,7-tetrahydro-1H-pyrazolo[4,3-c]pyridin-1-yl]propyl}piperidin-4-yl)pyrrolidin-2-one;
N-{1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-[4-(pyrrolidin-1-ylcarbonyl)piperidin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-phenylpiperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
N-[1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)piperidin-4-yl]methanesulfonamide;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)-4-pyridin-2-ylpiperidin-4-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)-4-pyridin-2-ylpiperidin-4-ol;
N-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyridin-2-yloxy)piperidin-1-yl]propan-2-ol;
1-[4-(2-Fluorophenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(2-pyridin-3-ylpiperidin-1-yl)propan-2-ol;
2-(4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperazin-1-yl)phenol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
1-[1-(3-{3-[3-{[2-(3,3-Dimethylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-[4-(pyrimidin-2-yloxy)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyrimidin-2-yloxy)piperidin-1-yl]propan-2-ol;
(2S)-1-(4-Cyclohexylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol; 1-[4-(2-Fluorophenyl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(3-Chloropyridin-2-yl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-Cyclopentyl-2-({5-[1-{2-hydroxy-3-[4-(5-oxo-4,5-dihydro-1H-1,2,4-triazol-3-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1,1-Dimethylethyl(2-{3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-oxoethyl)carbamate;
1-[4-(3-Methyl-1,2,4-oxadiazol-5-yl)piperidin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
2-(4-[2-Hydroxy-3-[5-(hydroxyacetyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
4-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)-3-methylpiperazin-2-one;
5-(1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol; N-Cyclopentyl-2-{[5-{1-[2-hydroxy-3-(4-morpholin-4-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}acetamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
1,1-Dimethylethyl 4-{[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl-sulfanyl)ethyl]aminopiperidine-1-carboxylate;
(2S)-1-(4-Cyclohexylpiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
5-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-2,4-dihydro-3H-1,2,4-triazol-3-one;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrazin-2-ylpiperazin-1-yl)propan-2-ol;
1-{3-[3-({2-[2-(Hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
(2S)-2-Hydroxy-N-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}propanamide;
5-Chloro-3-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-1-methyl-1,3-dihydro-2H-benzimidazol-2-one;
N-[4-(Dimethylamino)phenyl]-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
(4R)-1-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-4-hydroxypyrrolidin-2-one;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-phenylpiperidine-4-carbonitrile; (4S)-1-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-4-hydroxypyrrolidin-2-one;
1-[1-(3-{5-(Methylsulfonyl)-3-[3-({2-[(2-phenylethyl)amino]ethyl]sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
N-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]pyrrolidin-2-one;
1-{4-[2-(Methyloxy)phenyl]piperidin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]ethanesulfonamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
Phenylmethyl(2-{[5-{5-acetyl-1-[2-hydroxy-3-(4-pyridin-2-ylpiperidin-1-yl)propyl]-4,5,6,7-tetrahydro-1H-pyrazolo[4,3-c]pyridin-3-yl}-2-(trifluoromethyl)phenyl]sulfanyl}ethyl)carbamate;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
1-{4-[2-(Methyloxy)phenyl]piperazin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}-4-phenylpiperidin-4-ol;
1-{4-[2-(Methyloxy)phenyl]piperidin-1-yl}-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
N-{1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
N-[1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
N-(1-[2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
(3R)-1-[({5-[1-{2-Hydroxy-3-[4-(2-hydroxyphenyl)piperazin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-{4-[2-(Methyloxy)phenyl]piperazin-1-yl}-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl]sulfanyl)ethyl]piperidin-4-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
1-[4-(2-Chlorophenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
(3R)-1-[({5-[1-{3-[4-(4-Fluorophenyl)piperazin-1-yl]-2-hydroxypropyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-[4-(2-Fluorophenyl)piperazin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
2-[4-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]phenol; 1-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-pyridin-2-ylpiperidin-4-ol;
3-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}phenol;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]methanesulfonamide;
2-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}phenol;
N-{1-[(2S)-2-Hydroxy-3-{5-(methylsulfonyl)-3-[3-[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}acetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(piperidin-1-ylcarbonyl)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrimidin-2-yloxy)piperidin-1-yl]propan-2-ol;
1-[4-(3-Chloropyridin-2-yl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N-[2-({5-[1-{3-[4-(2-Cyanophenyl)piperazin-1-yl]-2-hydroxypropyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(1H-pyrrolo[2,3-b]pyridin-3-yl)piperidin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
(2S)-1-[4-(1-Hydroxy-1-methylethyl)piperidin-1-yl]-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propan-2-ol;
5-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-2,4-dihydro-3H-1,2,4-triazol-3-one; (3R)-1-[({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(piperidin-1-ylcarbonyl)piperidin-1-yl]propan-2-ol;
(2R)-1-[4-(2-Fluorophenyl)piperazin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol;
(4R)-4-Hydroxy-1-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
(4S)-4-Hydroxy-1-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[4-(3-Chloropyridin-2-yl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
4-Hydroxy-1-(1-[2-hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[1-(3-{3-[3-{[2-(2,3-Dihydro-1H-inden-2-ylamino)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol; 1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
2-Hydroxy-N-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}-2-methylpropanamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-morpholin-4-ylpiperidin-1-yl)propan-2-ol;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrimidin-2-yloxy)piperidin-1-yl]propan-2-ol;
2-(4-{2-Hydroxy-3-[5-(methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
4-[1-(2-Hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]benzonitrile;
1-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-4-hydroxypyrrolidin-2-one;
2-(4-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
2-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}benzonitrile;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
2-[4-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
1-{4-[2-(Ethyloxy)phenyl]piperazin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N′-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl]sulfanyl)ethyl]-N,N-dimethylsulfamide;
N-[1-(2-Hydroxy-3-{3-[3-{[2-(2-methyl-3-oxopiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-{4-[2-(Methyloxy)phenyl]piperidin-1-yl}-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-[2-({5-[1-(2-Hydroxy-3-{4-[2-(methyloxy)phenyl]piperidin-1-yl}propyl)-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol;
N-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
4-(2-{[5-{1-[2-Hydroxy-3-(4-hydroxy-4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)-3-methylpiperazin-2-one;
1,1-Dimethylethyl[1-({3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}carbonyl)cyclopropyl]carbamate;
2-[4-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperazin-1-yl]phenol;
1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
2-[4-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-{4-[2-(methyloxy)phenyl]piperidin-1-yl}propan-2-ol; N-{1-[2-({5-[5-(Methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)ethyl]piperidin-4-yl}acetamide;
N-[1-(2-Hydroxy-3-{3-[3-({2-[2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-(2-Hydroxy-3-{3-[3-(2-[2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
1-[1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]pyrrolidin-2-one;
N-Cyclopentyl-2-({5-[1-{2-hydroxy-3-[4-(morpholin-4-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetamide;
4-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]-3-methylpiperazin-2-one;
1-[4-(3-Chloropyridin-2-yl)piperazin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-morpholin-4-ylpiperidin-1-yl)propan-2-ol;
1-{3-[3-({2-[2-(Hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-{4-[(2-oxo-2-pyrrolidin-1-ylethyl)oxy]piperidin-1-yl}propan-2-ol;
(2R)-1-[4-(3-Chloropyridin-2-yl)piperazin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[1-(3-{3-[3-({2-[(1-Methyl-3-phenylpropyl)amino]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(morpholin-4-ylcarbonyl)piperidin-1-yl]propan-2-ol;
2-[4-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperazin-1-yl]benzonitrile;
2,2,2-Trifluoro-1-{2-[3-{3-[2-(4-fluoro-piperidin-1-yl)-ethylsulfanyl]-4-trifluoromethyl-phenyl}-1-(2-hydroxy-3-piperidin-1-yl-propyl)-1,4,6,7-tetrahydro-pyrazolo[4,3-c]pyridine-5-carbonyl]-pyrrolidin-1-yl}-ethanone;
N-[1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]methanesulfonamide;
1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
2-[4-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]phenol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
(2S)-2-Hydroxy-N-{1-[(2S)-2-hydroxy-3-[5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}propanamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(piperidin-1-ylcarbonyl)piperidin-1-yl]propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(morpholin-4-ylcarbonyl)piperidin-1-yl]propan-2-ol;
1-[1-(3-{5-(Methylsulfonyl)-3-[3-{[2-(3-phenylpiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
2-[4-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile; 1-[4-(3,5-Dichloropyridin-4-yl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1,1,1-Trifluoro-N-[2-({5-[1-{2-hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[4-(3,5-Dichloropyridin-4-yl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1,1-Dimethylethyl 4-{2-[1-(2-hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl]-2-oxoethyl}piperidine-1-carboxylate;
1-[1-(3-{3-[3-{[2-(1,4′-Bipiperidin-1′-yl)ethyl]sulfanyl-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
Methyl {1-[2-hydroxy-3-(4-pyridin-2-ylpiperidin-1-yl)propyl]-3-[3-{[2-({[(phenylmethyl)oxy]carbonyl]amino)ethyl]sulfanyl}-4-(trifluoromethyl)phenyl]-1,4,6,7-tetrahydro-5H-pyrazolo[4,3-c]pyridin-5-yl}(oxo)acetate;
1,1-Dimethylethyl 3-({3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}carbonyl)piperidine-1-carboxylate;
(4S)-4-Hydroxy-1-{1-[(2S)-2-hydroxy-3-[5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}pyrrolidin-2-one;
Ethyl {1-[2-({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl]sulfanyl)ethyl]piperidin-4-yl}carbamate;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]benzenesulfonamide;
1-[1-(3-{5-(Methylsulfonyl)-3-[3-({2-[4-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-({2-[2-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one; 1-[1-(3-{5-(Methylsulfonyl)-3-[3-({2-[3-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-{4-[1-(phenylmethyl)-1H-tetrazol-5-yl]piperidin-1-yl}propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-{4-[1-(phenylmethyl)-1H-tetrazol-5-yl]piperidin-1-yl}propan-2-ol;
1-(2-Hydroxy-3-{5-(methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}propyl)-4-phenylpiperidin-4-ol;
1-(2-Hydroxy-3-{5-(methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}propyl)-4-pyridin-2-ylpiperidin-4-ol;
4-(1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2H-1,4-benzoxazin-3(4H)-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
2-{1-[2-Hydroxy-3-(4-pyridin-2-ylpiperidin-1-yl)propyl]-3-[3-{[2-({[(phenylmethyl)oxy]carbonyl]amino)ethyl]sulfanyl}-4-(trifluoromethyl)phenyl]-1,4,6,7-tetrahydro-5H-pyrazolo[4,3-c]pyridin-5-yl}-2-oxoethyl acetate;
1,1-Dimethylethyl 4-(2-{3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-oxoethyl)piperidine-1-carboxylate;
5-Chloro-1-(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-benzimidazol-2-one;
Ethyl {1-[({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)acetyl]piperidin-4-yl}carbamate;
5-Chloro-1-methyl-3-(1-{3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-benzimidazol-2-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-[4-(piperidin-1-ylcarbonyl)piperidin-1-yl]propan-2-ol;
1,1-Dimethylethyl {1-[2-({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)ethyl]piperidin-4-yl}carbamate;
1,1-Dimethylethyl {1-[({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)acetyl]piperidin-4-yl}carbamate;
5-(Dimethylamino)-3-[1-(2-hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
5-(Dimethylamino)-3-[1-(2-hydroxy-3-{3-[3-({2-[(3S)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
1,1-Dimethylethyl 4-{2-[1-(2-hydroxy-3-{4-[(hydroxyacetyl)amino]piperidin-1-yl}propyl)-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl]-2-oxoethyl}piperidine-1-carboxylate; and
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-{4-[1-(phenylmethyl)-1H-tetrazol-5-yl]piperidin-1-yl}propan-2-ol;
and pharmaceutically acceptable salts thereof.

64. A method of treating a subject suffering from or diagnosed with a disease, disorder, or medical condition mediated by cathepsin S activity, comprising administering to a subject in need of such treatment an effective amount of at least one chemical entity selected from compounds of Formula (I), and pharmaceutically acceptable salts, prodrugs, and metabolites thereof: 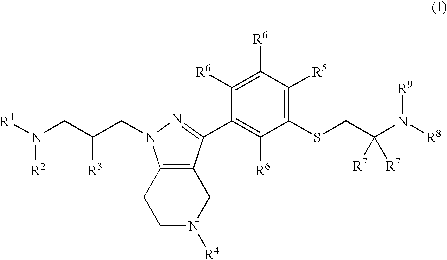
wherein:
R¹ and R² taken together with the nitrogen to which they are attached form a monocyclic heterocycloalkyl group, each group optionally containing one heteroatom ring member in addition to the nitrogen that is O, S, —N═, or NR^{a}, and each group unsubstituted or substituted with one, two, or three R^{b} substituents;
where R^{a} is H, C₁₋₄alkyl, —COC₁₋₄alkyl, or —CO₂C₁₋₄alkyl, or a phenyl ring, monocyclic cycloalkyl ring, or monocyclic heteroaryl ring, each ring unsubstituted or substituted with OH, C₁₋₄alkyl, CF₃, halo, —OC₁₋₄alkyl, cyano, or —COC₁₋₄alkyl; and
each R^{b} substituent is independently:
i) OH, C₁₋₄alkyl, CF₃, —NR^{c}R^{d}, halo, —OC₁₋₄alkyl, CH₂F, CHF₂, —C₁₋₄alkyl-OH, —CH₂NR^{c}R^{d}, —COC₁₋₄alkyl, —CO₂C₁₋₄alkyl, —CO₂H, —CONR^{m}Rⁿ, CN, or —OCH₂CONR^{m}Rⁿ;
ii) a monocyclic heterocycloalkyl group unsubstituted or substituted with C₁₋₄alkyl, OH, —OC₁₋₄alkyl, —NR^{c}R^{d}, or halo;
iii) a monocyclic heterocycloalkyl group fused with a phenyl or pyridyl group, the resulting fused bicyclic group being unsubstituted or substituted with C₁₋₄alkyl, OH, —OC₁₋₄alkyl, —NR^{c}R^{d}, or halo; or
iv) a phenyl group, benzyl group, monocyclic heteroaryl group, monocyclic cycloalkyl group, —O-phenyl group, or —O-(monocyclic heteroaryl) group, each group unsubstituted or substituted with C₁₋₄alkyl, OH, —OC₁₋₄alkyl, CF₃, cyano, —NR^{c}R^{d}, benzyl or halo; or
v) two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, —NR^{c}R^{d}, or halo; or
vi) two R^{b} substituents on adjacent carbons taken together with the carbons to which they are attached form a saturated monocyclic cycloalkyl, phenyl, or monocyclic heteroaryl group, each group unsubstituted or substituted with C₁₋₄alkyl, OH, —OC₁₋₄alkyl, —NR^{c}R^{d}, or halo;
where R^{c} and R^{d} are each independently H, C₁₋₄alkyl, —COC₁₋₄alkyl, —COC₁₋₄alkyl-OH, —CO₂C₁₋₄alkyl, —CONR^{x}R^{y}, —C₁₋₄alkyl-OH, or —SO₂C₁₋₄alkyl;
where R^{x} and R^{y} are each independently H or C₁₋₄alkyl; and
R^{m} and Rⁿ are each independently H or C₁₋₄alkyl; or R^{m} and Rⁿ taken together with the nitrogen to which they are attached form a monocyclic heterocycloalkyl ring;
R³ is H, OH, C₁₋₄alkyl, or —OC₁₋₄alkyl;
R⁴ is H; C₁₋₄alkyl; —COC₁₋₄alkyl unsubstituted or substituted with OH, F, —OCOC₁₋₄alkyl, —OC₁₋₄alkyl, phenyl, —SC₁₋₄alkyl, —NR^{o}R^{p}, or —N(R^{o})CO₂C₁₋₄alkyl; —CO-(monocyclic cycloalkyl); —CO-(monocyclic heteroaryl); —CO—(C-linked monocyclic heterocycloalkyl); —COCH₂— (monocyclic heterocycloalkyl); —SO₂C₁₋₄alkyl; —SO₂CF₃; —SO₂—(monocyclic cycloalkyl); —SO₂-phenyl; —SO₂-benzyl; —SO₂—NR^{o}R^{p}; —CONR^{o}R^{p}; —COCO₂C₁₋₄alkyl; —COCONH₂; or —COCONHC₁₋₄alkyl; wherein each cycloalkyl, heteroaryl, heterocycloalkyl, phenyl, or benzyl group is unsubstituted or substituted with C₁₋₄alkyl, OH, F, —COC₁₋₄alkyl, —COCF₃, —CO₂C₁₋₄alkyl, —N(R^{o})CO₂C₁₋₄alkyl, or —NR^{o}R^{p};
where R^{o} and R^{p} are each independently H or C₁₋₄alkyl, or a monocyclic cycloalkyl, phenyl, or monocyclic heteroaryl group; or R^{o} and R^{p} taken together with the nitrogen to which they are attached form a monocyclic heterocycloalkyl group; wherein each cycloalkyl, phenyl, heteroaryl, or heterocycloalkyl group is unsubstituted or substituted with C₁₋₄alkyl, OH, —OC₁₋₄alkyl, —NR^{s}R^{t}, or halo;
where R^{s} and R^{t} are each independently H, C₁₋₄alkyl, —COC₁₋₄alkyl, —CO₂C₁₋₄alkyl, —CONR^{v}R^{w}, C₁₋₄alkyl-OH, or —SO₂C₁₋₄alkyl;
where R^{v} and R^{w} are each independently H or C₁₋₄alkyl;
R⁵ is halo or CF₃;
each R⁶ is independently H or F;
each R⁷ is H or both R⁷ together form a carbonyl;
R⁸ is H or C₁₋₄alkyl and R⁹ is R¹⁰, —COR¹¹, —SO₂R¹¹, —SO₂CF₃, —SO₂N(C₁₋₄alkyl)₂, —CO₂C₁₋₄alkyl, or —CO₂-benzyl;
where R¹⁰ and R¹¹ are each: a C₁₋₄alkyl group unsubstituted or substituted with R^{e}; or a monocyclic cycloalkyl group, bicyclic cycloalkyl group, monocyclic heterocycloalkyl group, phenyl group, or fluorenyl group, each group unsubstituted or substituted with C₁₋₄alkyl, OH, —OC₁₋₄alkyl, —NR^{f}R^{s}, halo, —COC₁₋₄alkyl, —CO₂C₁₋₄alkyl, phenyl, benzyl, or benzyloxy;
where R^{e} is OH, —OC₁₋₄alkyl, or NR^{f}R^{g}, or a monocyclic cycloalkyl group, monocyclic heterocycloalkyl group, monocyclic heteroaryl group, phenyl group, or —O-phenyl group, each group unsubstituted or substituted with C₁₋₄alkyl, OH, —OC₁₋₄alkyl, —NR^{f}R^{g}, halo, —COC₁₋₄alkyl, or —CO₂C₁₋₄alkyl;
where R^{f} and R^{g} are each independently H, C₁₋₄alkyl, —COC₁₋₄alkyl, or —CO₂C₁₋₄alkyl;
or R⁸ and R⁹ taken together with the nitrogen to which they are attached form a monocyclic heterocycloalkyl group, each group optionally containing one heteroatom ring member in addition to the nitrogen that is O, S, SO₂, —N═, or NR^{h}, and each group unsubstituted or substituted with one or two Rⁱ substituents;
where R^{h} is H, C₁₋₄alkyl, phenyl, —C₁₋₂alkyl-phenyl, —COC₁₋₄alkyl, or —CO₂C₁₋₄alkyl; and
each Rⁱ substituent is independently: a C₁₋₄alkyl group unsubstituted or substituted with OH or phenyl; CF₃; halo; OH; —OC₁₋₄alkyl; —NR^{j}R^{k}; —COC₁₋₄alkyl; —CO₂C₁₋₄alkyl; —CONR^{j}R^{k}; phenyl; pyridyl; or benzyl; or two adjacent Rⁱ substituents taken together with the carbons to which they are attached form a phenyl or monocyclic cycloalkyl group;
where R^{j} and R^{k} are each independently H, C₁₋₄alkyl, —COC₁₋₄alkyl, —COC₁₋₄alkyl-OH, or —CO₂C₁₋₄alkyl; or R^{j} and R^{k} taken together with the nitrogen to which they are attached form a monocyclic saturated heterocycloalkyl ring.

65. A method according to claim 64, wherein said chemical entity is selected from the group consisting of:
1-{1-[3-(3-{3-[2-(4-Hydroxy-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(octahydro-isoquinolin-2-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4,4-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(3-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3,5-Dimethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(octahydro-quinolin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,6-Dihydro-2H-pyridin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(1,1-Dioxo-1λ⁶-thiomorpholin-4-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(4-tert-Butyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Fluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
(S)-1-{1-[3-(5-Methanesulfonyl-3-{4-trifluoromethyl-3-[2-(2-trifluoromethyl-pyrrolidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-[5-Methanesulfonyl-3-[3-(2-morpholin-4-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{3-[3-(2-Cyclopentylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-(1-{3-[3-(3-{2-[4-(2-Hydroxy-ethyl)-piperidin-1-yl]-ethylsulfanyl}-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one;
1-[1-(3-{5-Methanesulfonyl-3-[3-(2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-phenyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(Cyclohexyl-methyl-amino)-ethylsulfanyl]-4-trifluoromethyl-phenyl-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-pyrrolidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-[1-(3-{3-[3-(2-Benzylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-methylamino-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-[3-[2-(3-methoxy-propylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-[1-(3-{3-[3-(2-Dimethylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{3-[2-(4-Dimethylamino-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl}-piperidine-4-carboxylic acid amide;
1-{1-[3-(5-Methanesulfonyl-3-{3-[2-(4-methoxy-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{4-trifluoromethyl-3-[2-(3-trifluoromethyl-piperidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(2-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{4-trifluoromethyl-3-[2-(4-trifluoromethyl-piperidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-[1-(3-{5-Methanesulfonyl-3-[3-(2-thiazolidin-3-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-(1-{3-[5-Methanesulfonyl-3-(3-{2-[(thiophen-2-ylmethyl)-amino]-ethylsulfanyl}-4-trifluoromethyl-phenyl)-4,5,6,7-tetrahydro-pyrazolo[4,3-c]pyridin-1-yl]-propyl}-piperidin-4-yl)-pyrrolidin-2-one;
1-{1-[3-(3-{3-[2-(3-Hydroxy-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,4-Dihydro-1H-isoquinolin-2-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-[1,4]diazepan-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(2-Cyclohex-1-enyl-ethylamino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-phenoxy-ethylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-morpholin-4-yl-ethylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-phenethyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-(1-{3-[3-(3-{2-[(1-Ethyl-pyrrolidin-2-ylmethyl)-amino]-ethylsulfanyl}-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one;
1-{1-[3-(3-{3-[2-(Benzyl-methyl-amino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl}-piperidine-3-carboxylic acid diethylamide;
1-{1-[3-(3-{3-[2-(3-Dimethylamino-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
5-Methanesulfonyl-3-[3-(2-piperidin-1-yl-ethylsulfanyl)-4-trifluoromethyl-phenyl]-1-(3-piperidin-1-yl-propyl)-4,5,6,7-tetrahydro-1H-pyrazolo[4,3-c]pyridine;
5-Methanesulfonyl-3-[3-(2-piperidin-1-yl-ethylsulfanyl)-4-trifluoromethyl-phenyl]-1-[3-(4-pyrrolidin-1-yl-piperidin-1-yl)-propyl]-4,5,6,7-tetrahydro-1H-pyrazolo[4,3-c]pyridine;
1-[4-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperazin-1-yl]-ethanone;
5-Methanesulfonyl-3-[3-(2-piperidin-1-yl-ethylsulfanyl)-4-trifluoromethyl-phenyl]-1-(3-pyrrolidin-1-yl-propyl)-4,5,6,7-tetrahydro-1H-pyrazolo[4,3-c]pyridine;
1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-pyrrolidin-3-ol;
5-Methanesulfonyl-1-(3-morpholin-4-yl-propyl)-3-[3-(2-piperidin-1-yl-ethylsulfanyl)-4-trifluoromethyl-phenyl]-4,5,6,7-tetrahydro-1H-pyrazolo[4,3-c]pyridine;
4-[1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-4H-benzo[1,4]oxazin-3-one;
{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-carbamic acid tert-butyl ester;
1-[1-(2-Hydroxy-3-{5-methanesulfonyl-3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
N-Biphenyl-4-yl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-(2-phenoxy-ethyl)-acetamide;
1-{1-[2-Hydroxy-3-(5-methanesulfonyl-3-{3-[2-oxo-2-(4-phenyl-3,6-dihydro-2H-pyridin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-indan-2-yl-acetamide;
N-Biphenyl-3-yl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-(9H-Fluoren-1-yl)-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide; N-(4-Benzyloxy-phenyl)-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-Benzyl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-phenethyl-acetamide;
1-{1-[3-(3-{3-[2-(3-Hydroxy-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-pyrrolidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxymethyl-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxy-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(3-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-[3-[2-(octahydro-isoquinolin-2-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Fluoro-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
(S)-1-{1-[3-(5-Methanesulfonyl-3-{3-[2-oxo-2-(2-trifluoromethyl-pyrrolidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one; N-(4-Fluoro-phenyl)-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetamide;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-N,N-dimethyl-acetamide;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-N-methyl-acetamide;
1-{1-[3-(3-{3-[2-(4-Dimethylamino-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Dimethylamino-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[3-(2-[1,4′]Bipiperidinyl-1′-yl-2-oxo-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetyl}-piperidine-4-carboxylic acid amide;
1-[1-(3-{5-Methanesulfonyl-3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-oxo-2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-morpholin-4-yl-2-oxo-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{3-[3-(2-Azepan-1-yl-2-oxo-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-methyl-[1,4]diazepan-1-yl)-2-oxo-ethylsulfanyl]-4-trifluoromethyl-phenyl}-4,5,6,7-tetrahydro-pyrazolo[4,3c]pyridin-1-yl)-propyl]-piperidin-4-yl}-pyrrolidin-2-one;
1-{1-[3-(3-{3-[2-(3,6-Dihydro-2H-pyridin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,4-Dihydro-1H-isoquinolin-2-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethylphenylsulfanyl]-N-methyl-N-(1-methyl-piperidin-4-yl)-acetamide;
1-{1-[3-(5-Methanesulfonyl-3-{3-[2-oxo-2-(4-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
N-Cyclohexyl-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethylphenylsulfanyl]-N-methyl-acetamide;
N-Benzyl-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetamide;
1-{1-[3-(5-Methanesulfonyl-3-{3-[2-(4-methyl-piperazin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Benzyl-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-oxo-2-(4-phenethyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-oxo-2-(4-phenyl-3,6-dihydro-2H-pyridin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl phenylsulfanyl]-acetyl}-piperidine-4-carboxylic acid diethylamide;
(R)-1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetyl}-pyrrolidine-2-carboxylic acid amide;
1-{1-[3-(3-{3-[2-(4-Isopropyl-piperazin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
2-(5-{5-Methanesulfonyl-1-[3-(4-pyrrolidin-1-yl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone;
2-(5-{5-Methanesulfonyl-1-[3-(4-trifluoromethyl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone;
2-(5-{1-[3-(4-Acetyl-piperazin-1-yl)-propyl]-5-methanesulfonyl-4,5,6,7-tetrahydro-1H-pyrazolo[4,3-c]pyridin-3-yl}-2-trifluoromethyl-phenylsulfanyl)-1-morpholin-4-yl-ethanone;
-(3-[5-Methanesulfonyl-3-[3-(2-morpholin-4-yl-2-oxo-ethylsulfanyl)-4-trifluoromethyl-phenyl]-4,5,6,7-tetrahydro-pyrazolo[4,3-c]pyridin-1-yl}-propyl)-piperidin-4-yl]-carbamic acid tert-butyl ester;
N-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
N-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-2-phenyl-acetamide;
4-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-piperidine-1-carboxylic acid tert-butyl ester;
({2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-methyl)-carbamic acid tert-butyl ester;
4-Fluoro-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
4-Hydroxy-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
4-Dimethylamino-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
2-(4-Fluoro-phenyl)-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-acetamide;
3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-piperidine-1-carboxylic acid tert-butyl ester;
3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-pyrrolidine-1-carboxylic acid tert-butyl ester; (3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-propyl)-carbamic acid tert-butyl ester;
Piperidine-3-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
Pyrrolidine-3-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
Piperidine-4-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
(2S)-1-(4,4-Dimethyl-piperidin-1-yl)-3-(3-{3-[2-(4-fluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propan-2-ol;
(2S)—N-{1-[3-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-acetamide; and
(2S)-1-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-3-(4-methylpiperidin-1-yl)-propan-2-ol;
and pharmaceutically acceptable salts thereof.

66. A method according to claim 64, wherein said chemical entity is selected from the group consisting of:
1-[1-(3-{3-[3-(2-Amino-ethylsulfanyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-pyrrolidin-2-one;
1-[1-(3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
N-[1-(3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-acetamide;
5-Dimethylamino-3-[1-(3-{3-[3-(2-hydroxy-ethylsulfanyl)-4-trifluoromethyl-phenyl]-5-methanesulfonyl-4,5,6,7-tetrahydro-pyrazolo[4,3-c]pyridin-1-yl}-propyl)piperidin-4-yl]-1-methyl-1,3-dihydro-imidazo[4,5-b]pyridin-2-one;
[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid ethyl ester; [5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid;
3-(3-Benzylsulfanyl-4-chloro-phenyl)-5-methanesulfonyl-1-(3-morpholin-4-yl-propyl)-4,5,6,7-tetrahydro-1H-pyrazolo[4,3-c]pyridine;
3-(4-Chloro-3-phenethylsulfanyl-phenyl)-5-methanesulfonyl-1-(3-morpholin-4-yl-propyl)-4,5,6,7-tetrahydro-1H-pyrazolo[4,3-c]pyridine;
5-Methanesulfonyl-1-(3-morpholin-4-yl-propyl)-3-[3-(2-phenoxy-ethylsulfanyl)-4-trifluoromethyl-phenyl]-4,5,6,7-tetrahydro-1H-pyrazolo[4,3-c]pyridine;
3-[4-Chloro-3-(furan-2-ylmethylsulfanyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-propylsulfanyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(2-phenoxy-ethylsulfanyl)-phenyl]-5-methanesulfonyl-1-(3-morpholin-4-yl-propyl)-4,5,6,7-tetrahydro-1H-pyrazolo[4,3-c]pyridine;
1-[1-(3-{3-[3-(4-Chloro-benzylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
3-[4-Chloro-3-(4-chloro-benzylsulfanyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-fluoro-benzylsulfanyl)-phenyl]-5-methanesulfonyl-1-(3-morpholin-4-yl-propyl)-4,5,6,7-tetrahydro-1H-pyrazolo[4,3-c]pyridine;
1-[1-(3-{5-Methanesulfonyl-3-[3-(2-phenoxy-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
3-[4-Chloro-3-(2-pyrazin-2-yl-ethylsulfanyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-chloro-propylsulfanyl)-phenyl]-5-methanesulfonyl-1-(3-morpholin-4-yl-propyl)-4,5,6,7-tetrahydro-1H-pyrazolo[4,3-c]pyridine; and
3-(4-Chloro-3-phenylmethanesulfinyl-phenyl)-5-methanesulfonyl-1-(3-morpholin-4-yl-propyl)-4,5,6,7-tetrahydro-1H-pyrazolo[4,3-c]pyridine;
and pharmaceutically acceptable salts thereof.

67. A method according to claim 64, wherein said chemical entity is selected from the group consisting of:
1-[1-(3-{3-[3-({2-[(1,1-Dimethylethyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
3-[3-({2-[(3S)-3-Methylmorpholin-4-yl]ethyl]sulfanyl)-4-(trifluoromethyl)phenyl]-5-(methylsulfonyl)-1-(3-piperidin-1-ylpropyl)-4,5,6,7-tetrahydro-1H-pyrazolo[4,3-c]pyridine; 3-[3-({2-[(2R,6R)-2,6-Dimethylmorpholin-4-yl]ethyl]sulfanyl)-4-(trifluoromethyl)phenyl]-5-(methylsulfonyl)-1-(3-piperidin-1-ylpropyl)-4,5,6,7-tetrahydro-1H-pyrazolo[4,3-c]pyridine;
{4-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]morpholin-2-yl}methanol;
{(3S)-4-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]morpholin-3-yl]methanol;
5-(Dimethylamino)-1-methyl-3-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-imidazo[4,5-b]pyridin-2-one;
Ethyl(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
N-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]propan-1-amine;
N-[2-({5-[5-(Methylsulfonyl)-1-{3-[4-(piperidin-1-ylcarbonyl)piperidin-1-yl]propyl}-4,5,6,7-tetrahydro-1H-pyrazolo[4,3-c]pyridin-3-yl]-2-(trifluoromethyl)phenyl}sulfanyl)ethyl]propan-1-amine;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
5-(Dimethylamino)-3-(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1-methyl-1,3-dihydro-2H-imidazo[4,5-b]pyridin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
2-(4-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-(4-Cyclopropylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-morpholin-4-ylpropan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-pyrrolidin-1-ylpropan-2-ol; 1-[4-(Dimethylamino)piperidin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-(4-Methylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-({2-[2-(Hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-[1-(2-Hydroxy-3-{3-[3-({2-[2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
4-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)ethyl]-3-methylpiperazin-2-one;
4-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]-3-methylpiperazin-2-one;
4-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)-3-methylpiperazin-2-one;
1-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)ethyl]piperidin-4-ol;
1-[1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
2-[4-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperazin-1-yl]benzonitrile;
1-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
5-(Dimethylamino)-3-[1-(2-hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-(4-Methylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4,4-Dimethylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4-Fluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(1,1-Dimethylethyl)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-morpholin-4-ylpropan-2-ol;
5-(Dimethylamino)-3-(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1-methyl-1,3-dihydro-2H-imidazo[4,5-b]pyridin-2-one;
1-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
2-(4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{5-(Ethylsulfonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl]sulfanyl)ethyl]piperidin-3-ol;
1-(2-{[5-{1-[2-Hydroxy-3-(4-hydroxy-4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-3-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(2-hydroxyethyl)piperidin-1-yl]propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxamide;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-2-hydroxyacetamide;
1-{1-[(2S)-2-Hydroxy-3-{3-[3-({2-[(2R)-2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[(2S)-2-Hydroxy-3-[5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]-4-methylpiperidin-4-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(octahydroisoquinolin-2(1H)-yl)propan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrazin-2-ylpiperazin-1-yl)propan-2-ol;
(2R)-1-[4-(3,5-Dichloropyridin-4-yl)piperidin-1-yl]-3-{3-[3-{[2-(4-fluoropiperidin-1-yl)ethyl]sulfanyl-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(3,6-Dihydropyridin-1(2H)-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol; (2S)-1-[4-(1,1-Dimethylethyl)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
N-{1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1,1-dimethylethyl)piperidin-1-yl]propan-2-ol;
(2S)-1-(4-Cyclohexylpiperidin-1-yl)-3-{3-[3-{[2-(4,4-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(Hexahydrocyclopenta[c]pyrrol-2(1H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-methylpiperidin-4-ol;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidine-4-carboxamide;
(2S)-1-{3-[3-({2-[4-(1,1-Dimethylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol; N-[1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-yl]acetamide;
2-Hydroxy-N-[1-(2-{[5-{1-[(2S)-2-hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-yl]acetamide;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)-4-pyridin-2-ylpiperidin-4-ol;
(2S)-1-{3-[3-({2-[(3S)-3-Methylmorpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(2S)-1-{3-[3-{[2-(Hexahydrocyclopenta[c]pyrrol-2(1H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)-4-methylpiperidin-4-ol;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidine-4-carboxamide;
(2S)-1-{3-[3-({2-[4-(1-Hydroxy-1-methylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
5-(Dimethylamino)-3-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-1-methyl-1,3-dihydro-2H-imidazo[4,5-b]pyridin-2-one;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyrrolidin-1-ylpiperidin-1-yl)propan-2-ol;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-(1-{2-Hydroxy-3-[5-(phenylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-(1-{3-[5-(Ethylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-2-hydroxypropyl}piperidin-4-yl)pyrrolidin-2-one;
1-[5-(Ethylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-{1-[2-Hydroxy-3-(5-[(phenylmethyl)sulfonyl]-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-[5-(Hydroxyacetyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-[5-(Hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-(1-{2-Hydroxy-3-[5-(hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)piperidin-4-yl]pyrrolidin-2-one;
1-[3-{3-[(2-Morpholin-4-ylethyl)sulfanyl]-4-(trifluoromethyl)phenyl}-5-(piperidin-4-ylacetyl)-4,5,6,7-tetrahydro-1H-pyrazolo[4,3-c]pyridin-1-yl]-3-piperidin-1-ylpropan-2-ol;
Methyl[1-(2-hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl](oxo)acetate;
Methyl(1-{2-hydroxy-3-[4-(2-oxopyrrolidin-1-yl)piperidin-1-yl]propyl}-3-{3-[(2-piperidin-1-ylethyl)sulfanyl]-4-(trifluoromethyl)phenyl}-1,4,6,7-tetrahydro-5H-pyrazolo[4,3-c]pyridin-5-yl)(oxo)acetate;
1-(5-Acetyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-{1-[3-(5-Acetyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}pyrrolidin-2-one;
1-(1-{3-[5-(Furan-2-ylcarbonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-2-hydroxypropyl}piperidin-4-yl)pyrrolidin-2-one;
1-(5-Methyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
2-(1-{(2S)-3-[4-(Acetylamino)piperidin-1-yl]-2-hydroxypropyl}-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
2-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-2-oxoacetamide;
(2S)-1-{3-[3-{[2-(4-Fluoropiperidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-piperidin-1-ylpropan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-methyl-1,4,6,7-tetrahydro-5H-pyrazolo[4,3-c]pyridine-5-carboxamide;
5-(Methylsulfonyl)-3-{3-[(2-morpholin-4-yl-2-oxoethyl)sulfanyl]-4-(trifluoromethyl)phenyl}-1-(3-piperidin-1-ylpropyl)-4,5,6,7-tetrahydro-1H-pyrazolo[4,3-c]pyridine;
5-(Methylsulfonyl)-3-{3-[(2-morpholin-4-yl-2-oxoethyl)sulfanyl]-4-(trifluoromethyl)phenyl}-1-(3-morpholin-4-ylpropyl)-4,5,6,7-tetrahydro-1H-pyrazolo[4,3-c]pyridine;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-morpholin-4-ylpiperidin-1-yl)propan-2-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-{4-[2-(Methyloxy)phenyl]piperidin-1-yl}-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propan-2-ol;
1-{1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-(3-{3-[(2-Morpholin-4-yl-2-oxoethyl)sulfanyl]-4-(trifluoromethyl)phenyl]-4,5,6,7-tetrahydro-1H-pyrazolo[4,3-c]pyridin-1-yl)-3-(4-pyridin-2-ylpiperidin-1-yl)propan-2-ol;
1-[4-(1,1-Dimethylethyl)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyridin-2-ylpiperidin-1-yl)propan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-{4-[2-(methyloxy)phenyl]piperidin-1-yl}propan-2-ol;
1-{3-[3-{[2-(4-Fluoropiperidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-(4-pyridin-2-ylpiperidin-1-yl)propan-2-ol;
1-(5-Acetyl-3-{3-[(2-morpholin-4-yl-2-oxoethyl)sulfanyl]-4-(trifluoromethyl)phenyl-4,5,6,7-tetrahydro-1H-pyrazolo[4,3-c]pyridin-1-yl)-3-{4-[2-(methyloxy)phenyl]piperidin-1-yl}propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(1,1-dimethylethyl)piperidin-1-yl]propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-methylpiperidin-1-yl)propan-2-ol;
1-{1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}pyrrolidin-2-one; and
1-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
and pharmaceutically acceptable salts thereof.

68. A method according to claim 64, wherein said chemical entity is selected from the group consisting of:
1-[3-{3-[2-(4-Fluoro-piperidin-1-yl)-ethylsulfanyl]-4-trifluoromethyl-phenyl}-1-(2-hydroxy-3-piperidin-1-yl-propyl)-1,4,6,7-tetrahydro-pyrazolo[4,3-c]pyridin-5-yl]-2-hydroxy-ethanone;
(2S)-1-(4-Cyclohexyl-piperidin-1-yl)-3-(3-{3-[2-((3S)-fluoro-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propan-2-ol;
N-[1-(2-{5-[1-((2S)-Hydroxy-3-morpholin-4-yl-propyl)-5-methanesulfonyl-4,5,6,7-tetrahydro-1H-pyrazolo[4,3-c]pyridin-3-yl]-2-trifluoromethyl-phenylsulfanyl}-ethyl)-piperidin-4-yl]-acetamide;
(2S)-1-(3-{3-[2-((3S)-Fluoro-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-3-(3′,4′,5′,6′-tetrahydro-2′H-[2,4′]bipyridinyl-1′-yl)-propan-2-ol;
(3S)-1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-3-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-pyrrolidin-1-ylpropan-2-ol;
1-Piperidin-1-yl-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]pyrrolidin-3-ol;
1-(3-{3-[(2-Morpholin-4-ylethyl)sulfanyl]-4-(trifluoromethyl)phenyl-4,5,6,7-tetrahydro-1H-pyrazolo[4,3-c]pyridin-1-yl)-3-piperidin-1-ylpropan-2-ol;
1-Morpholin-4-yl-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(4-Methylpiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-ol;
1-[2-({5-[1-(2-Hydroxy-3-piperidin-1-ylpropyl)-4,5,6,7-tetrahydro-1H-pyrazolo[4,3-c]pyridin-3-yl]-2-(trifluoromethyl)phenyl}sulfanyl)ethyl]piperidin-3-ol;
1-(4-Fluoropiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-morpholin-4-ylpropan-2-ol;
1-(Hexahydrocyclopenta[c]pyrrol-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol
1-(4,4-Dimethylpiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]-4-methylpiperidin-4-ol;
1-[4-(Methyloxy)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(4-Fluoropiperidin-1-yl)ethyl]sulfanyl}-4-(trifluoromethyl)phenyl]-5-methyl-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-methylpiperidin-1-yl)propan-2-ol;
1-(1,3-Dihydro-2H-isoindol-2-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
3-{3-[(2-Azetidin-1-ylethyl)sulfanyl]-4-(trifluoromethyl)phenyl}-5-(methylsulfonyl)-1-(3-piperidin-1-ylpropyl)-4,5,6,7-tetrahydro-1H-pyrazolo[4,3-c]pyridine;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidine-4-carboxamide;
1-(4-Acetylpiperazin-1-yl)-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidine-4-carboxamide;
1-(3,3-Difluoropyrrolidin-1-yl)-3-{3-[3-{[2-(3,3-difluoropyrrolidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-(4,4-Dimethylpiperidin-1-yl)-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
5-(Methylsulfonyl)-1-(3-piperidin-1-ylpropyl)-3-{3-[(2-pyrrolidin-1-ylethyl)sulfanyl]-4-(trifluoromethyl)phenyl}-4,5,6,7-tetrahydro-1H-pyrazolo[4,3-c]pyridine;
1-(3,4-Dihydroisoquinolin-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
N-Methyl-N-[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]propan-2-amine;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[(3S)-3-(trifluoromethyl)pyrrolidin-1-yl]propan-2-ol;
1-(Octahydroisoquinolin-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
2-(1-[(2S)-2-Hydroxy-3-piperidin-1-ylpropyl]-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
1-(1,4-Dioxa-8-azaspiro[4.5]dec-8-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[4-(1-Hydroxy-1-methylethyl)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}urea;
(2S)-1-{5-Acetyl-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidine-4-carboxamide;
1-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]imidazolidin-4-one;
1-{3-[3-[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
5-(Methylsulfonyl)-3-{3-[(2-morpholin-4-ylethyl)sulfanyl]-4-(trifluoromethyl)phenyl}-1-(3-piperidin-1-ylpropyl)-4,5,6,7-tetrahydro-1H-pyrazolo[4,3-c]pyridine;
1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidine-4-carboxamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-pyrrolidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(hexahydrocyclopenta[c]pyrrol-2(1H)-yl)propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4,4-dimethylpiperidin-1-yl)propan-2-ol;
1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidine-4-carboxamide;
2-Hydroxy-N-{1-[2-hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
2-Hydroxy-N-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
(2S)-1-[1-(2-Hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl]-1-oxopropan-2-ol;
1-(5-[(Methyloxy)acetyl]-3-{3-[(2-morpholin-4-ylethyl)sulfanyl]-4-(trifluoromethyl)phenyl]-4,5,6,7-tetrahydro-1H-pyrazolo[4,3-c]pyridin-1-yl)-3-piperidin-1-ylpropan-2-ol; 1-[2-({5-[5-(Hydroxyacetyl)-1-(2-hydroxy-3-piperidin-1-ylpropyl)-4,5,6,7-tetrahydro-1H-pyrazolo[4,3-c]pyridin-3-yl]-2-(trifluoromethyl)phenyl]sulfanyl)ethyl]piperidin-3-ol;
(2S)-1-{3-[3-{[2-(4-Fluoropiperidin-1-yl)ethyl]sulfanyl}-4-(trifluoromethyl)phenyl]-5-propanoyl-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-glycyl-4,5,6,7-tetrahydro-1H-pyrazolo[4,3-c]pyridin-1-yl}-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(1,3-dihydro-2H-isoindol-2-yl)propan-2-ol;
N-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]cyclohexanamine;
1-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}urea;
N-(2-{[5-{1-[3-(4-Cyclopropylpiperazin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)propan-1-amine;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-4-ylpiperidin-1-yl)propan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
1-(4-Methylpiperidin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(4-Methylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}pyrrolidin-3-ol;
1-[(3S)-3-(Hydroxymethyl)pyrrolidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[(3S)-3-Methylmorpholin-4-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]urea; (2S)-1-(5,6-Dihydropyrimidin-1(4H)-yl)-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-morpholin-4-ylpropan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}pyrrolidin-3-ol;
5-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-2,4-dihydro-3H-1,2,4-triazol-3-one;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
2-{1-[(2S)-2-Hydroxy-3-piperidin-1-ylpropyl]-3-[3-{[2-(4-methylpiperidin-1-yl)ethyl]sulfanyl}-4-(trifluoromethyl)phenyl]-1,4,6,7-tetrahydro-5H-pyrazolo[4,3-c]pyridin-5-yl}-2-oxoacetamide;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-(4-pyrrolidin-1-ylpiperidin-1-yl)propan-2-ol;
1-(4-Morpholin-4-ylpiperidin-1-yl)-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
2-{1-(2-Hydroxy-3-piperidin-1-ylpropyl)-3-[3-{[2-(4-methylpiperidin-1-yl)ethyl]sulfanyl}-4-(trifluoromethyl)phenyl]-1,4,6,7-tetrahydro-5H-pyrazolo[4,3-c]pyridin-5-yl}-2-oxoacetamide;
N-{1-[(2S)-3-(5-Acetyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}acetamide;
(4R)-1-{1-[(2S)-3-(5-Acetyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}-4-hydroxypyrrolidin-2-one;
Ethyl {1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}carbamate;
N-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)ethyl]methanesulfonamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-dimethyl-1,4,6,7-tetrahydro-5H-pyrazolo[4,3-c]pyridine-5-carboxamide;
1-(5-[(Methylsulfanyl)acetyl]-3-{3-[(2-morpholin-4-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(3,4-dihydroisoquinolin-2(1H)-yl)propan-2-ol;
(2S)-1-{3-[3-{[2-(4-Fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-1-oxopropan-2-ol;
(2S)-1-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-1-oxopropan-2-ol;
1-{3-[3-{[2-(4-Fluoropiperidin-1-yl)ethyl]sulfanyl}-4-(trifluoromethyl)phenyl]-5-[(methyloxy)acetyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-[(methyloxy)acetyl]-4,5,6,7-tetrahydro-1H-pyrazolo[4,3-c]pyridin-1-yl}-3-piperidin-1-ylpropan-2-ol;
3-Methyl-4-[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]piperazin-2-one;
N-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}-2-hydroxyacetamide;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(4,4-Dimethylpiperidin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
2-{4-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
N-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
1-(4-Methyl-1,4-diazepan-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
3-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
4-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
2-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}-4-methylpiperidin-4-ol;
(2S)-1-(3,6-Dihydropyridin-1(2H)-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
(3S)-1-[({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)acetyl]pyrrolidin-3-ol;
(3R)-1-[({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)acetyl]pyrrolidin-3-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(2S)-1-{3-[3-({2-[(3S)-3-Methylmorpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(3S)-1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-3-ol;
(2S)-1-(5,6-Dihydropyrimidin-1(4H)-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(5,6-Dihydropyrimidin-1(4H)-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-(4-Methylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(4-Fluorophenyl)piperazin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
N-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]-2-hydroxyacetamide;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-4-ylpiperidin-1-yl)propan-2-ol;
1-[4-(2-Fluorophenyl)piperazin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol; (2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
1-[4-(4-Fluorophenyl)piperazin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-Azepan-1-yl-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
(3R)-1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-3-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-ol;
(3S)-1-[2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}pyrrolidin-3-ol;
(3R)-1-[2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}pyrrolidin-3-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(3S)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-ol;
N-Methyl-2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)-N-(phenylmethyl)ethanamine;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperidin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-[1-(2-Hydroxy-3-{3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
(4R)-4-Hydroxy-1-{1-[(2S)-2-hydroxy-3-(5-methyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(1-hydroxy-1-methylethyl)piperidin-1-yl]propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(1,4-dioxa-8-azaspiro[4.5]dec-8-yl)propan-2-ol;
(2S)-1-{5-(Cyclobutylcarbonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
4-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
3-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
4-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
1-{5-[(1-Aminocyclopropyl)carbonyl]-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}pyrrolidin-2-one;
N-(2-{[5-{1′-[2-Hydroxy-3-(4-methylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)methanesulfonamide;
2-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}benzonitrile;
2-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-N-methyl-2-oxoacetamide;
1-{5-(N,N-Dimethylglycyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol 3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-(1-methylethyl)-1,4,6,7-tetrahydro-5H-pyrazolo[4,3-c]pyridine-5-carboxamide;
Methyl {3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}(oxo)acetate;
Methyl {3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}(oxo)acetate;
1-{3-[3-{[2-(4-Fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-methyl-1-oxopropan-2-ol;
1-(4-Cyclopropylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]pyrrolidin-2-one;
N-(2-{[5-{5-(Methylsulfonyl)-1-[3-(4-pyrrolidin-1-ylpiperidin-1-yl)propyl]-4,5,6,7-tetrahydro-1H-pyrazolo[4,3-c]pyridin-3-yl}-2-(trifluoromethyl)phenyl]sulfanyl]ethyl)propan-1-amine;
(2S)-1-{3-[3-{[2-(4-Fluoropiperidin-1-yl)ethyl]sulfanyl}-4-(trifluoromethyl)phenyl]-5-[(methylsulfanyl)acetyl]-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-[(methylsulfanyl)acetyl]-4,5,6,7-tetrahydro-1H-pyrazolo[4,3-c]pyridin-1-yl}-3-piperidin-1-ylpropan-2-ol;
1-{4-[2-(Methyloxy)phenyl]piperidin-1-yl}-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-(4-Methylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-Azepan-1-yl-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{4-[4-(Methyloxy)phenyl]piperidin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[2-(Methyloxy)phenyl]piperidin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
1-(4-Acetylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol; N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}pyrrolidin-3-yl)acetamide;
1-{4-[3-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[4-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[2-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}praline;
(2S)-1-[(3S)-3-Methylmorpholin-4-yl]-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(3,3-Dimethylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-3-methylpiperazin-2-one;
N-{1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]-4-pyridin-2-ylpiperidin-4-ol;
1-(2-{[5-{1-[2-Hydroxy-3-(4-hydroxy-4-pyridin-2-ylpiperidin-1-yl)propyl]-4,5,6,7-tetrahydro-1H-pyrazolo[4,3-c]pyridin-3-yl}-2-(trifluoromethyl)phenyl]sulfanyl]ethyl)piperidin-3-ol;
N-[1-(2-Hydroxy-3-{3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
(2S)-1-(4,4-Dimethylpiperidin-1-yl)-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
1-[(3S)-3-(Hydroxymethyl)pyrrolidin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol; 1-[(3S)-3-Methylmorpholin-4-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
1-[4-(3-Chlorophenyl)piperidin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[4-(Methyloxy)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-Azepan-1-yl-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-Azepan-1-yl-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
1-(2-{[5-(1-{2-Hydroxy-3-[4-(pyrimidin-2-yloxy)piperidin-1-yl]propyl}-4,5,6,7-tetrahydro-1H-pyrazolo[4,3-c]pyridin-3-yl)-2-(trifluoromethyl)phenyl]sulfanyl]ethyl)piperidin-3-ol;
1-(4-Aminopiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
N-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}methanesulfonamide;
2-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}phenol;
3-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}phenol;
1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-ol; (3S)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-3-ol;
1-[2-(Hydroxymethyl)morpholin-4-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-3-ol;
1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(3,6-dihydropyridin-1(2H)-yl)propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-3-ol;
(3R)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-3-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-ol;
N-Methyl-2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)-N-(2-phenylethyl)ethanamine;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[(2S)-2-(hydroxymethyl)pyrrolidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(5,6-dihydropyrimidin-1(4H)-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(5,6-dihydropyrimidin-1(4H)-yl)propan-2-ol; (2S)-1-{3-[3-{[2-(4-Fluoropiperidin-1-yl)ethyl]sulfanyl}-4-(trifluoromethyl)phenyl]-5-(1H-1,2,4-triazol-3-ylcarbonyl)-4,5,6,7-tetrahydro-1H-pyrazolo[4,3-c]pyridin-1-yl}-3-piperidin-1-ylpropan-2-ol;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
1-[4-(Morpholin-4-ylcarbonyl)piperidin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(2-{[5-(1-{2-Hydroxy-3-[4-(pyrrolidin-1-ylcarbonyl)piperidin-1-yl]propyl}-4,5,6,7-tetrahydro-1H-pyrazolo[4,3-c]pyridin-3-yl)-2-(trifluoromethyl)phenyl]sulfanyl]ethyl)piperidin-3-ol;
(2S)-1-[4-(2-Fluorophenyl)piperazin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-[4-(4-Fluorophenyl)piperazin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-(3-Fluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-(4-Fluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(4-Fluoropiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
N-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]methanesulfonamide;
4-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]phenol;
1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)-4-pyridin-2-ylpiperidin-4-ol;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
3-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]phenol; 2-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]pyridine-3-carbonitrile;
(2S)-1-{3-[3-{[2-(4-Fluoropiperidin-1-yl)ethyl]sulfanyl}-4-(trifluoromethyl)phenyl]-5-(pyrrolidin-1-ylcarbonyl)-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
N-(2-{[5-{1-[3-(4,4-Dimethylpiperidin-1-yl)-2-hydroxypropyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-(4,4-Difluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(3,3-Difluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
1-{3-[3-{[2-(4-Fluoropiperidin-1-yl)ethyl]sulfanyl}-4-(trifluoromethyl)phenyl]-5-[(2R)-tetrahydrofuran-2-ylcarbonyl]-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(4-fluorophenyl)piperazin-1-yl]propan-2-ol;
N-(1,1-Dimethylethyl)-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-(1-methylpropyl)-1,4,6,7-tetrahydro-5H-pyrazolo[4,3-c]pyridine-5-carboxamide; 1-[1-(3-{3-[3-{[2-(Cyclopropylamino)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
N,N-Diethyl-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
2-{4-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperazin-1-yl}benzonitrile;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(2,4,6,7-tetrahydro-5H-pyrazolo[4,3-c]pyridin-5-yl)propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(1,4,6,7-tetrahydro-5H-imidazo[4,5-c]pyridin-5-yl)propan-2-ol;
N-(1-{3-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
1-[1-(3-{3-[3-({2-[(1-Methylethyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-{[2-(propylamino)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
4-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}benzonitrile;
1-(4-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenyl)ethanone;
N,N-Dimethyl-1-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-3-yl)methanamine;
3-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}benzonitrile;
2-[4-(3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]benzonitrile;
2-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}benzonitrile; 1-[4-(1,1-Dimethylethyl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-(4,4-Dimethylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{4-[2-(Methyloxy)phenyl]piperidin-1-yl}-3-(5-methyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(4-Cyclopropylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4-Acetylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
2-{4-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperazin-1-yl}phenol;
1-[4-(1-Methylethyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{4-[2-(Ethyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{5-(Cyclopropylsulfonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[4-(1-Hydroxy-1-methylethyl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]-4-methylpiperidin-4-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
N-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}pyrrolidin-3-yl)acetamide;
1-(4-Acetylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(Dimethylamino)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol; 1-[4-(1-Methylethyl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidine-4-carboxamide;
1-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(4-fluorophenyl)piperazin-1-yl]propan-2-ol;
(2S)-1-{3-[3-{[2-(4-Fluoropiperidin-1-yl)ethyl]sulfanyl}-4-(trifluoromethyl)phenyl]-5-(propylsulfonyl)-4,5,6,7-tetrahydro-1H-pyrazolo[4,3-c]pyridin-1-yl}-3-piperidin-1-ylpropan-2-ol
(2S)-1-{3-[3-{[2-(4-Fluoropiperidin-1-yl)ethyl]sulfanyl}-4-(trifluoromethyl)phenyl]-5-[(1-methylethyl)sulfonyl]-4,5,6,7-tetrahydro-1H-pyrazolo[4,3-c]pyridin-1-yl}-3-piperidin-1-ylpropan-2-ol;
(2S)-1-(4,4-Dimethylpiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
2-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]benzonitrile;
(2R)-1-(4,4-Dimethylpiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-(4,4-Dimethylpiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(methyloxy)phenyl]piperidin-1-yl}propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[4-(methyloxy)phenyl]piperidin-1-yl}propan-2-ol;
1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-dimethyl-1,4,6,7-tetrahydro-5H-pyrazolo[4,3-c]pyridine-5-sulfonamide;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(methyloxy)phenyl]piperazin-1-yl}propan-2-ol; 1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4-Fluoropiperidin-1-yl)ethyl]sulfanyl}-4-(trifluoromethyl)phenyl]-5-[(1-methyl-1H-imidazol-2-yl)carbonyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(hydroxymethyl)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(hydroxymethyl)piperidin-1-yl]propan-2-ol;
1-(5-Acetyl-3-{3-[(2-morpholin-4-yl-2-oxoethyl)sulfanyl]-4-(trifluoromethyl)phenyl-4,5,6,7-tetrahydro-1H-pyrazolo[4,3-c]pyridin-1-yl)-3-(4-pyrrolidin-1-ylpiperidin-1-yl)propan-2-ol;
1-(3,4-Dihydroisoquinolin-2(1H)-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-Azepan-1-yl-3-{3-[3-{[2-(3,3-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-Azepan-1-yl-3-{3-[3-{[2-(4,4-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(2-thienylcarbonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(2-thienylcarbonyl)-4,5,6,7-tetrahydro-1H-pyrazolo[4,3-c]pyridin-1-yl}-3-piperidin-1-ylpropan-2-ol;
(2S)-1-[4-(3-Chlorophenyl)piperidin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
N-[1-(3-{5-(Methylsulfonyl)-3-[3-{[2-(propylamino)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[3-(methyloxy)phenyl]piperazin-1-yl}propan-2-ol;
1-{3-[3-{[2-(4-Fluoropiperidin-1-yl)ethyl]sulfanyl}-4-(trifluoromethyl)phenyl]-5-(1-methyl-L-prolyl)-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[4-(methyloxy)phenyl]piperazin-1-yl}propan-2-ol;
1-{3-[3-{[2-(4-Fluoropiperidin-1-yl)ethyl]sulfanyl}-4-(trifluoromethyl)phenyl]-5-(piperidin-3-ylcarbonyl)-4,5,6,7-tetrahydro-1H-pyrazolo[4,3-c]pyridin-1-yl}-3-piperidin-1-ylpropan-2-ol;
4-{1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}benzonitrile;
2-Hydroxy-N-{1-[2-hydroxy-3-(5-[(methyloxy)acetyl]-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
(2S)-1-{3-[3-{[2-(4-Fluoropiperidin-1-yl)ethyl]sulfanyl}-4-(trifluoromethyl)phenyl]-5-(morpholin-4-ylcarbonyl)-4,5,6,7-tetrahydro-1H-pyrazolo[4,3-c]pyridin-1-yl}-3-piperidin-1-ylpropan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-(4,4-Difluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{5-(Methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[1-(3-{3-[3-({2-[(2-Methylpropyl)amino]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-pyrrolidin-1-ylpropan-2-ol;
(2S)-1-{5-(Methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}-3-morpholin-4-ylpropan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(octahydroisoquinolin-2(1H)-yl)propan-2-ol;
N-[(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methyl]acetamide; 1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidine-4-carboxamide;
1-[1-(3-{3-[3-({2-[(3-Hydroxypropyl)amino]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[5-(Hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)urea;
1-(1,5-Dioxa-9-azaspiro[5.5]undec-9-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-{3-[3-({2-[4-(1-Hydroxy-1-methylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
2-Hydroxy-N-(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-phenyl-1,4,6,7-tetrahydro-5H-pyrazolo[4,3-c]pyridine-5-carboxamide;
(2S)-1-[4-(1,1-Dimethylethyl)piperidin-1-yl]-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
N-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
(2S)-1-[4-(Dimethylamino)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
(2S)-1-{4-[2-(Ethyloxy)phenyl]piperazin-1-yl}-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxylic acid;
(3S)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-3-carboxylic acid;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-3-carboxylic acid;
(3R)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-3-carboxylic acid;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-phenylpiperidin-1-yl)propan-2-ol;
1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-3-carboxylic acid;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-yl]acetamide;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(2-hydroxyethyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4,4-dimethylpiperidin-1-yl)propan-2-ol; (2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4,4-dimethylpiperidin-1-yl)propan-2-ol;
N-[1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-yl]acetamide;
1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-2-thienyl-1,4,6,7-tetrahydro-5H-pyrazolo[4,3-c]pyridine-5-carboxamide;
1-{3-[3-{[2-(4-Fluoropiperidin-1-yl)ethyl]sulfanyl}-4-(trifluoromethyl)phenyl]-5-(piperidin-4-ylacetyl)-4,5,6,7-tetrahydro-1H-pyrazolo[4,3-c]pyridin-1-yl}-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyrrolidin-1-ylpiperidin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(ethyloxy)phenyl]piperazin-1-yl}propan-2-ol;
N-Cyclohexyl-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4-Fluoropiperidin-1-yl)ethyl]sulfanyl}-4-(trifluoromethyl)phenyl]-5-[(4-methylpiperazin-1-yl)carbonyl]-4,5,6,7-tetrahydro-1H-pyrazolo[4,3-c]pyridin-1-yl}-3-piperidin-1-ylpropan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-{4-[3-(trifluoromethyl)phenyl]piperazin-1-yl}propan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-bis(1-methylethyl)-1,4,6,7-tetrahydro-5H-pyrazolo[4,3-c]pyridine-5-carboxamide;
(2S)-1-[4-(1,1-Difluoroethyl)piperidin-1-yl]-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol; (2S)-1-(4-Methylpiperidin-1-yl)-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propan-2-ol;
2-Hydroxy-N-{1-[2-hydroxy-3-(5-[(methylsulfanyl)acetyl]-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
1-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-(1,4′-Bipiperidin-1′-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(trifluoromethyl)piperidin-1-yl]propan-2-ol;
(2S)-1-[4-(1,1-Dimethylethyl)piperidin-1-yl]-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol;
N-{1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}acetamide;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
(2S)-1-{3-[3-{[2-(4-Fluoropiperidin-1-yl)ethyl]sulfanyl}-4-(trifluoromethyl)phenyl]-5-[(trifluoromethyl)sulfonyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(octahydroquinolin-1(2H)-yl)propan-2-ol;
(2S)-1-[4-(1-Hydroxy-1-methylethyl)piperidin-1-yl]-3-{3-[3-{[2-(4-methylpiperidin-1-yl)ethyl]sulfanyl-4-(trifluoromethyl)phenyl]-5-(methylsulfonyl)-4,5,6,7-tetrahydro-1H-pyrazolo[4,3-c]pyridin-1-yl}propan-2-ol;
N-[(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)methyl]acetamide;
Ethyl(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
(2S)-1-[4-(1,1-Dimethylethyl)piperidin-1-yl]-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(4-Fluoropiperidin-1-yl)ethyl]sulfanyl}-4-(trifluoromethyl)phenyl]-5-[(2S)-2-hydroxy-2-phenylacetyl]-4,5,6,7-tetrahydro-1H-pyrazolo[4,3-c]pyridin-1-yl}-3-piperidin-1-ylpropan-2-ol;
2-Hydroxy-N-[1-(2-{[5-{1-[(2S)-2-hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-yl]acetamide;
N-{1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
2-Hydroxy-N-(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
N-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-phenylpiperazin-1-yl)propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-4-ylpiperidin-1-yl)propan-2-ol;
1-(5-Acetyl-3-{3-[(2-morpholin-4-yl-2-oxoethyl)sulfanyl]-4-(trifluoromethyl)phenyl-4,5,6,7-tetrahydro-1H-pyrazolo[4,3-c]pyridin-1-yl)-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-phenylpiperidin-1-yl)propan-2-ol;
2-(1-[(2S)-2-Hydroxy-3-{4-[(4R)-4-hydroxy-2-oxopyrrolidin-1-yl]piperidin-1-yl}propyl]-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyridin-4-ylpiperidin-1-yl)propan-2-ol; 1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-phenylpiperazin-1-yl)propan-2-ol;
N-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
1-[1-(2-Hydroxy-3-{5-(hydroxyacetyl)-3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
1-{5-(1-Acetyl-L-prolyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyrimidin-2-ylpiperazin-1-yl)propan-2-ol;
1-[1-(3-[3-[3-{[2-(Cyclohexylamino)ethyl]sulfanyl]-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]-4-[3-(trifluoromethyl)phenyl]piperidin-4-ol;
2-(4-{2-Hydroxy-3-[5-(hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
5-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-Hydroxy-1-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[4-(3-Methyl-1,2,4-oxadiazol-5-yl)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-cyclopropylpiperazin-1-yl)propan-2-ol;
5-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-Hydroxy-1-(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyrrolidin-1-ylpiperidin-1-yl)propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[3-(trifluoromethyl)phenyl]piperazin-1-yl}propan-2-ol;
N-(2-{[5-{1′-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
Ethyl(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
N-(1-[2-Hydroxy-3-[5-(hydroxyacetyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
2-Hydroxy-N-[1-(2-hydroxy-3-{3-[3-({2-[(3S)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
2-Hydroxy-N-(1-[2-hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
2-Hydroxy-N-[1-(2-hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
Ethyl(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
2-Hydroxy-N-[1-(2-hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
N-[1-(2-Hydroxy-3-{5-(hydroxyacetyl)-3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
(2S)-1-(1,5-Dioxa-9-azaspiro[5.5]undec-9-yl)-3-{3-[3-[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[4-(2-Methylphenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{4-[2-(Methyloxy)phenyl]piperidin-1-yl}-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(phenylmethyl)piperidin-1-yl]propan-2-ol;
N-{1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
1-(2-{[5-{1-[2-Hydroxy-3-(4-phenylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1,1-dimethylethyl)piperidin-1-yl]propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
(3S)-1-({[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]acetyl)pyrrolidin-3-ol;
N-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-(2-{[5-{1-[2-Hydroxy-3-(4-phenylpiperazin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
(3R)-1-({[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]acetyl)pyrrolidin-3-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyridin-4-ylpiperidin-1-yl)propan-2-ol;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)ethanesulfonamide;
1-(3-{3-[3-[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyridin-2-yloxy)piperidin-1-yl]propan-2-ol;
2-(4-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)phenol;
1-(1-{3-[3-{3-[(2-{[2-(1H-imidazol-4-yl)ethyl]amino]ethyl)sulfanyl]-4-(trifluoromethyl)phenyl}-5-(methylsulfonyl)-4,5,6,7-tetrahydro-1H-pyrazolo[4,3-c]pyridin-1-yl]propyl}piperidin-4-yl)pyrrolidin-2-one; N-{1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-[4-(pyrrolidin-1-ylcarbonyl)piperidin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-phenylpiperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
N-[1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)piperidin-4-yl]methanesulfonamide;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)-4-pyridin-2-ylpiperidin-4-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)-4-pyridin-2-ylpiperidin-4-ol;
N-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyridin-2-yloxy)piperidin-1-yl]propan-2-ol;
1-[4-(2-Fluorophenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol; (2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(2-pyridin-3-ylpiperidin-1-yl)propan-2-ol;
2-(4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperazin-1-yl)phenol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
1-[1-(3-{3-[3-{[2-(3,3-Dimethylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-[4-(pyrimidin-2-yloxy)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyrimidin-2-yloxy)piperidin-1-yl]propan-2-ol;
(2S)-1-(4-Cyclohexylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[4-(2-Fluorophenyl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(3-Chloropyridin-2-yl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-Cyclopentyl-2-({5-[1-{2-hydroxy-3-[4-(5-oxo-4,5-dihydro-1H-1,2,4-triazol-3-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1,1-Dimethylethyl(2-{3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-oxoethyl)carbamate; 1-[4-(3-Methyl-1,2,4-oxadiazol-5-yl)piperidin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
2-(4-[2-Hydroxy-3-[5-(hydroxyacetyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
4-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)-3-methylpiperazin-2-one;
5-(1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
1-[5-(Methylsulfonyl)-3-{3-[(2-morpholin-4-ylethyl)sulfanyl]-4-(trifluoromethyl)phenyl]-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
N-Cyclopentyl-2-{[5-{1-[2-hydroxy-3-(4-morpholin-4-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}acetamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
1,1-Dimethylethyl 4-{[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl-sulfanyl)ethyl]amino]piperidine-1-carboxylate;
(2S)-1-(4-Cyclohexylpiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
5-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-2,4-dihydro-3H-1,2,4-triazol-3-one;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrazin-2-ylpiperazin-1-yl)propan-2-ol;
1-{3-[3-({2-[2-(Hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
(2S)-2-Hydroxy-N-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}propanamide;
5-Chloro-3-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-1-methyl-1,3-dihydro-2H-benzimidazol-2-one;
N-[4-(Dimethylamino)phenyl]-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
(4R)-1-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-4-hydroxypyrrolidin-2-one;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-phenylpiperidine-4-carbonitrile;
(4S)-1-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-4-hydroxypyrrolidin-2-one;
1-[1-(3-{5-(Methylsulfonyl)-3-[3-({2-[(2-phenylethyl)amino]ethyl]sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
N-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]pyrrolidin-2-one;
1-{4-[2-(Methyloxy)phenyl]piperidin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol; N-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]ethanesulfonamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
Phenylmethyl(2-{[5-{5-acetyl-1-[2-hydroxy-3-(4-pyridin-2-ylpiperidin-1-yl)propyl]-4,5,6,7-tetrahydro-1H-pyrazolo[4,3-c]pyridin-3-yl}-2-(trifluoromethyl)phenyl]sulfanyl}ethyl)carbamate;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
1-{4-[2-(Methyloxy)phenyl]piperazin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}-4-phenylpiperidin-4-ol;
1-{4-[2-(Methyloxy)phenyl]piperidin-1-yl}-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
N-{1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
N-[1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
N-(1-[2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide; (3R)-1-[({5-[1-{2-Hydroxy-3-[4-(2-hydroxyphenyl)piperazin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-{4-[2-(Methyloxy)phenyl]piperazin-1-yl}-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl]sulfanyl)ethyl]piperidin-4-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
1-[4-(2-Chlorophenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
(3R)-1-[({5-[1-{3-[4-(4-Fluorophenyl)piperazin-1-yl]-2-hydroxypropyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-[4-(2-Fluorophenyl)piperazin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
2-[4-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]phenol;
1-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-pyridin-2-ylpiperidin-4-ol;
3-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}phenol;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]methanesulfonamide;
2-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}phenol;
N-{1-[(2S)-2-Hydroxy-3-{5-(methylsulfonyl)-3-[3-[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}acetamide; 1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(piperidin-1-ylcarbonyl)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrimidin-2-yloxy)piperidin-1-yl]propan-2-ol;
1-[4-(3-Chloropyridin-2-yl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N-[2-({5-[1-{3-[4-(2-Cyanophenyl)piperazin-1-yl]-2-hydroxypropyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(1H-pyrrolo[2,3-b]pyridin-3-yl)piperidin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
(2S)-1-[4-(1-Hydroxy-1-methylethyl)piperidin-1-yl]-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propan-2-ol;
5-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-2,4-dihydro-3H-1,2,4-triazol-3-one;
(3R)-1-[({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(piperidin-1-ylcarbonyl)piperidin-1-yl]propan-2-ol;
(2R)-1-[4-(2-Fluorophenyl)piperazin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol;
(4R)-4-Hydroxy-1-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one; 1-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
(4S)-4-Hydroxy-1-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[4-(3-Chloropyridin-2-yl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
4-Hydroxy-1-(1-[2-hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[1-(3-{3-[3-{[2-(2,3-Dihydro-1H-inden-2-ylamino)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
2-Hydroxy-N-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}-2-methylpropanamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-morpholin-4-ylpiperidin-1-yl)propan-2-ol;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrimidin-2-yloxy)piperidin-1-yl]propan-2-ol;
2-(4-{2-Hydroxy-3-[5-(methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile; 4-[1-(2-Hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]benzonitrile;
1-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-4-hydroxypyrrolidin-2-one;
2-(4-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
2-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}benzonitrile;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
2-[4-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
1-{4-[2-(Ethyloxy)phenyl]piperazin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N′-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl]sulfanyl)ethyl]-N,N-dimethylsulfamide;
N-[1-(2-Hydroxy-3-{3-[3-{[2-(2-methyl-3-oxopiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-{4-[2-(Methyloxy)phenyl]piperidin-1-yl}-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-[2-({5-[1-(2-Hydroxy-3-{4-[2-(methyloxy)phenyl]piperidin-1-yl}propyl)-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol; N-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
4-(2-{[5-{1-[2-Hydroxy-3-(4-hydroxy-4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)-3-methylpiperazin-2-one;
1,1-Dimethylethyl[1-({3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}carbonyl)cyclopropyl]carbamate;
2-[4-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperazin-1-yl]phenol;
1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
2-[4-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-{4-[2-(methyloxy)phenyl]piperidin-1-yl}propan-2-ol;
N-{1-[2-({5-[5-(Methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl]sulfanyl)ethyl]piperidin-4-yl}acetamide;
N-[1-(2-Hydroxy-3-{3-[3-({2-[2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-(2-Hydroxy-3-{3-[3-(2-[2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
1-[1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]pyrrolidin-2-one;
N-Cyclopentyl-2-({5-[1-{2-hydroxy-3-[4-(morpholin-4-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetamide; 4-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl]sulfanyl)ethyl]-3-methylpiperazin-2-one;
1-[4-(3-Chloropyridin-2-yl)piperazin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-morpholin-4-ylpiperidin-1-yl)propan-2-ol;
1-{3-[3-({2-[2-(Hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-{4-[(2-oxo-2-pyrrolidin-1-ylethyl)oxy]piperidin-1-yl}propan-2-ol;
(2R)-1-[4-(3-Chloropyridin-2-yl)piperazin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[1-(3-{3-[3-({2-[(1-Methyl-3-phenylpropyl)amino]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(morpholin-4-ylcarbonyl)piperidin-1-yl]propan-2-ol;
2-[4-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperazin-1-yl]benzonitrile;
2,2,2-Trifluoro-1-{2-[3-{3-[2-(4-fluoro-piperidin-1-yl)-ethylsulfanyl]-4-trifluoromethyl-phenyl}-1-(2-hydroxy-3-piperidin-1-yl-propyl)-1,4,6,7-tetrahydro-pyrazolo[4,3-c]pyridine-5-carbonyl]-pyrrolidin-1-yl}-ethanone;
N-[1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]methanesulfonamide;
1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol; 2-[4-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]phenol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
(2S)-2-Hydroxy-N-{1-[(2S)-2-hydroxy-3-[5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}propanamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(piperidin-1-ylcarbonyl)piperidin-1-yl]propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(morpholin-4-ylcarbonyl)piperidin-1-yl]propan-2-ol;
1-[1-(3-{5-(Methylsulfonyl)-3-[3-{[2-(3-phenylpiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
2-[4-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
1-[4-(3,5-Dichloropyridin-4-yl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1,1,1-Trifluoro-N-[2-({5-[1-{2-hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[4-(3,5-Dichloropyridin-4-yl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1,1-Dimethylethyl 4-{2-[1-(2-hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl]-2-oxoethyl}piperidine-1-carboxylate;
1-[1-(3-{3-[3-{[2-(1,4′-Bipiperidin-1′-yl)ethyl]sulfanyl-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
Methyl {1-[2-hydroxy-3-(4-pyridin-2-ylpiperidin-1-yl)propyl]-3-[3-{[2-({[(phenylmethyl)oxy]carbonyl-4-amino)ethyl]sulfanyl}-4-(trifluoromethyl)phenyl]-1,4,6,7-tetrahydro-5H-pyrazolo[4,3-c]pyridin-5-yl}(oxo)acetate;
1,1-Dimethylethyl 3-({3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}carbonyl)piperidine-1-carboxylate;
(4S)-4-Hydroxy-1-{1-[(2S)-2-hydroxy-3-[5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}pyrrolidin-2-one;
Ethyl {1-[2-({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl]sulfanyl)ethyl]piperidin-4-yl}carbamate;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]benzenesulfonamide;
1-[1-(3-{5-(Methylsulfonyl)-3-[3-({2-[4-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-({2-[2-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-({2-[3-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-{4-[1-(phenylmethyl)-1H-tetrazol-5-yl]piperidin-1-yl}propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-{4-[1-(phenylmethyl)-1H-tetrazol-5-yl]piperidin-1-yl}propan-2-ol;
1-(2-Hydroxy-3-{5-(methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}propyl)-4-phenylpiperidin-4-ol;
1-(2-Hydroxy-3-{5-(methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}propyl)-4-pyridin-2-ylpiperidin-4-ol;
4-(1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2H-1,4-benzoxazin-3(4H)-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
2-{1-[2-Hydroxy-3-(4-pyridin-2-ylpiperidin-1-yl)propyl]-3-[3-{[2-({[(phenylmethyl)oxy]carbonyl-4-amino)ethyl]sulfanyl}-4-(trifluoromethyl)phenyl]-1,4,6,7-tetrahydro-5H-pyrazolo[4,3-c]pyridin-5-yl}-2-oxoethyl acetate;
1,1-Dimethylethyl 4-(2-{3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-oxoethyl)piperidine-1-carboxylate;
5-Chloro-1-(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-benzimidazol-2-one;
Ethyl {1-[({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)acetyl]piperidin-4-yl}carbamate;
5-Chloro-1-methyl-3-(1-{3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-benzimidazol-2-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-[4-(piperidin-1-ylcarbonyl)piperidin-1-yl]propan-2-ol;
1,1-Dimethylethyl {1-[2-({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)ethyl]piperidin-4-yl}carbamate;
1,1-Dimethylethyl {1-[({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)acetyl]piperidin-4-yl}carbamate;
5-(Dimethylamino)-3-[1-(2-hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
5-(Dimethylamino)-3-[1-(2-hydroxy-3-{3-[3-({2-[(3S)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
1,1-Dimethylethyl 4-{2-[1-(2-hydroxy-3-{4-[(hydroxyacetyl)amino]piperidin-1-yl}propyl)-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl]-2-oxoethyl}piperidine-1-carboxylate; and
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-{4-[1-(phenylmethyl)-1H-tetrazol-5-yl]piperidin-1-yl}propan-2-ol;
and pharmaceutically acceptable salts thereof.

69. A method according to claim 64, wherein the disease, disorder, or medical condition is an autoimmune disease, an allergic condition, inflammation, a bowel disorder, tissue transplant rejection, pain, or cancer.

70. A method according to claim 64, wherein the disease, disorder, or medical condition is selected from the group consisting of: lupus, asthma, allergic reaction, atopic allergy, hay fever, atopic dermatitis, food allergy, rhinitis, skin immune system disorders, psoriasis, uveitis, inflammation, upper airway inflammation, Sjögren's syndrome, arthritis, rheumatoid arthritis, osteoarthritis, type I diabetes, atherosclerosis, multiple sclerosis, coeliac disease, inflammatory bowel disease, chronic obstructive pulmonary disorder, tissue transplant rejection, pain, chronic pain, and cancer.

71. A method according to claim 64, wherein the disease, disorder, or medical condition is selected from the group consisting of: psoriasis, pain, multiple sclerosis, atherosclerosis, and rheumatoid arthritis.

72. A method for modulating cathepsin S activity, comprising exposing cathepsin S to an effective amount of at least one chemical entity selected from compounds of Formula (I), pharmaceutically acceptable salts of compounds of Formula (I), pharmaceutically acceptable prodrugs of compounds of Formula (I), and pharmaceutically active metabolites of compounds of Formula (I): 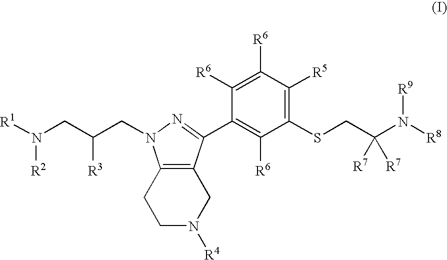
wherein:
R¹ and R² taken together with the nitrogen to which they are attached form a monocyclic heterocycloalkyl group, each group optionally containing one heteroatom ring member in addition to the nitrogen that is O, S, —N═, or NR^{a}, and each group unsubstituted or substituted with one, two, or three R^{b} substituents;
where R^{a} is H, C₁₋₄alkyl, —COC₁₋₄alkyl, or —CO₂C₁₋₄alkyl, or a phenyl ring, monocyclic cycloalkyl ring, or monocyclic heteroaryl ring, each ring unsubstituted or substituted with OH, C₁₋₄alkyl, CF₃, halo, —OC₁₋₄alkyl, cyano, or —COC₁₋₄alkyl; and
each R^{b} substituent is independently:
i) OH, C₁₋₄alkyl, CF₃, —NR^{c}R^{d}, halo, —OC₁₋₄alkyl, CH₂F, CHF₂, —C₁₋₄alkyl-OH, —CH₂NR^{c}R^{d}, —COC₁₋₄alkyl, —CO₂C₁₋₄alkyl, —CO₂H, —CONR^{m}Rⁿ, CN, or —OCH₂CONR^{m}Rⁿ;
ii) a monocyclic heterocycloalkyl group unsubstituted or substituted with C₁₋₄alkyl, OH, —OC₁₋₄alkyl, —NR^{c}R^{d}, or halo;
iii) a monocyclic heterocycloalkyl group fused with a phenyl or pyridyl group, the resulting fused bicyclic group being unsubstituted or substituted with C₁₋₄alkyl, OH, —OC₁₋₄alkyl, —NR^{c}R^{d}, or halo; or
iv) a phenyl group, benzyl group, monocyclic heteroaryl group, monocyclic cycloalkyl group, —O-phenyl group, or —O-(monocyclic heteroaryl) group, each group unsubstituted or substituted with C₁₋₄alkyl, OH, —OC₁₋₄alkyl, CF₃, cyano, —NR^{c}R^{d}, benzyl or halo; or
v) two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, —NR^{c}R^{d}, or halo; or
vi) two R^{b} substituents on adjacent carbons taken together with the carbons to which they are attached form a saturated monocyclic cycloalkyl, phenyl, or monocyclic heteroaryl group, each group unsubstituted or substituted with C₁₋₄alkyl, OH, —OC₁₋₄alkyl, —NR^{c}R^{d}, or halo;
where R^{c} and R^{d} are each independently H, C₁₋₄alkyl, —COC₁₋₄alkyl, —COC₁₋₄alkyl-OH, —CO₂C₁₋₄alkyl, —CONR^{x}R^{y}, —C₁₋₄alkyl-OH, or —SO₂C₁₋₄alkyl;
where R^{x} and R^{y} are each independently H or C₁₋₄alkyl; and
R^{m} and Rⁿ are each independently H or C₁₋₄alkyl; or R^{m} and Rⁿ taken together with the nitrogen to which they are attached form a monocyclic heterocycloalkyl ring;
R³ is H, OH, C₁₋₄alkyl, or —OC₁₋₄alkyl;
R⁴ is H; C₁₋₄alkyl; —COC₁₋₄alkyl unsubstituted or substituted with OH, F, —OCOC₁₋₄alkyl, —OC₁₋₄alkyl, phenyl, —SC₁₋₄alkyl, —NR^{o}R^{p}, or —N(R^{o})CO₂C₁₋₄alkyl; —CO-(monocyclic cycloalkyl); —CO-(monocyclic heteroaryl); —CO—(C-linked monocyclic heterocycloalkyl); —COCH₂— (monocyclic heterocycloalkyl); —SO₂C₁₋₄alkyl; —SO₂CF₃; —SO₂—(monocyclic cycloalkyl); —SO₂-phenyl; —SO₂-benzyl; —SO₂—NR^{o}R^{p}; —CONR^{o}R^{p}; —COCO₂C₁₋₄alkyl; —COCONH₂; or —COCONHC₁₋₄alkyl; wherein each cycloalkyl, heteroaryl, heterocycloalkyl, phenyl, or benzyl group is unsubstituted or substituted with C₁₋₄alkyl, OH, F, —COC₁₋₄alkyl, —COCF₃, —CO₂C₁₋₄alkyl, —N(R^{o})CO₂C₁₋₄alkyl, or —NR^{o}R^{p};
where R^{o} and R^{p} are each independently H or C₁₋₄alkyl, or a monocyclic cycloalkyl, phenyl, or monocyclic heteroaryl group; or R^{o} and R^{p} taken together with the nitrogen to which they are attached form a monocyclic heterocycloalkyl group; wherein each cycloalkyl, phenyl, heteroaryl, or heterocycloalkyl group is unsubstituted or substituted with C₁₋₄alkyl, OH, —OC₁₋₄alkyl, —NR^{s}R^{t}, or halo;
where R^{s} and R^{t} are each independently H, C₁₋₄alkyl, —COC₁₋₄alkyl, —CO₂C₁₋₄alkyl, —CONR^{v}R^{w}, C₁₋₄alkyl-OH, or —SO₂C₁₋₄alkyl;
where R^{v} and R^{w} are each independently H or C₁₋₄alkyl;
R⁵ is halo or CF₃;
each R⁶ is independently H or F;
each R⁷ is H or both R⁷ together form a carbonyl;
R⁸ is H or C₁₋₄alkyl and R⁹ is R¹⁰, —COR¹¹, —SO₂R¹¹, —SO₂CF₃, —SO₂N(C₁₋₄alkyl)₂, —CO₂C₁₋₄alkyl, or —CO₂-benzyl;
where R¹⁰ and R¹¹ are each: a C₁₋₄alkyl group unsubstituted or substituted with R^{e}; or a monocyclic cycloalkyl group, bicyclic cycloalkyl group, monocyclic heterocycloalkyl group, phenyl group, or fluorenyl group, each group unsubstituted or substituted with C₁₋₄alkyl, OH, —OC₁₋₄alkyl, —NR^{f}R^{g}, halo, —COC₁₋₄alkyl, —CO₂C₁₋₄alkyl, phenyl, benzyl, or benzyloxy;
where R^{e} is OH, —OC₁₋₄alkyl, or NR^{f}R^{g}, or a monocyclic cycloalkyl group, monocyclic heterocycloalkyl group, monocyclic heteroaryl group, phenyl group, or —O-phenyl group, each group unsubstituted or substituted with C₁₋₄alkyl, OH, —OC₁₋₄alkyl, —NR^{f}R^{g}, halo, —COC₁₋₄alkyl, or —CO₂C₁₋₄alkyl;
where R^{f} and R^{g} are each independently H, C₁₋₄alkyl, —COC₁₋₄alkyl, or —CO₂C₁₋₄alkyl;
or R⁸ and R⁹ taken together with the nitrogen to which they are attached form a monocyclic heterocycloalkyl group, each group optionally containing one heteroatom ring member in addition to the nitrogen that is O, S, SO₂, —N═, or NR^{h}, and each group unsubstituted or substituted with one or two Rⁱ substituents;
where R^{h} is H, C₁₋₄alkyl, phenyl, —C₁₋₂alkyl-phenyl, —COC₁₋₄alkyl, or —CO₂C₁₋₄alkyl; and
each Rⁱ substituent is independently: a C₁₋₄alkyl group unsubstituted or substituted with OH or phenyl; CF₃; halo; OH; —OC₁₋₄alkyl; —NR^{j}R^{k}; —COC₁₋₄alkyl; —CO₂C₁₋₄alkyl; —CONR^{j}R^{k}; phenyl; pyridyl; or benzyl; or two adjacent Rⁱ substituents taken together with the carbons to which they are attached form a phenyl or monocyclic cycloalkyl group;
where R^{j} and R^{k} are each independently H, C₁₋₄alkyl, —COC₁₋₄alkyl, —COC₁₋₄alkyl-OH, or —CO₂C₁₋₄alkyl; or R^{j} and R^{k} taken together with the nitrogen to which they are attached form a monocyclic saturated heterocycloalkyl ring.

73. A method according to claim 72, wherein the cathepsin S is in a subject with a disease, disorder, or medical condition mediated by cathepsin S activity.

74. A method according to claim 73, wherein the disease, disorder, or medical condition is an autoimmune disease, an allergic condition, inflammation, a bowel disorder, tissue transplant rejection, pain, or cancer.

75. A method according to claim 73, wherein the disease, disorder, or medical condition is selected from the group consisting of: lupus, asthma, allergic reaction, atopic allergy, hay fever, atopic dermatitis, food allergy, rhinitis, skin immune system disorders, psoriasis, uveitis, inflammation, upper airway inflammation, Sjögren's syndrome, arthritis, rheumatoid arthritis, osteoarthritis, type I diabetes, atherosclerosis, multiple sclerosis, coeliac disease, inflammatory bowel disease, chronic obstructive pulmonary disorder, tissue transplant rejection, pain, chronic pain, and cancer.

76. A method as in claim 73, wherein the disease, disorder, or medical condition is psoriasis, pain, multiple sclerosis, atherosclerosis, or rheumatoid arthritis.

77. A method according to claim 72, wherein said chemical entity is selected from the group consisting of:
1-{1-[3-(3-{3-[2-(4-Hydroxy-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-[3-[2-(octahydro-isoquinolin-2-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4,4-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(3-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3,5-Dimethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(octahydro-quinolin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,6-Dihydro-2H-pyridin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(1,1-Dioxo-1λ⁶-thiomorpholin-4-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(4-tert-Butyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Fluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
(S)-1-{1′-[3-(5-Methanesulfonyl-3-{4-trifluoromethyl-3-[2-(2-trifluoromethyl-pyrrolidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{5-Methanesulfonyl-3-[3-(2-morpholin-4-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{3-[3-(2-Cyclopentylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-(1-{3-[3-(3-{2-[4-(2-Hydroxy-ethyl)-piperidin-1-yl]-ethylsulfanyl}-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one;
1-[1-(3-{5-Methanesulfonyl-3-[3-(2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-phenyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-[3-[2-(Cyclohexyl-methyl-amino)-ethylsulfanyl]-4-trifluoromethyl-phenyl]-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-pyrrolidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-[1-(3-{3-[3-(2-Benzylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-methylamino-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-[3-[2-(3-methoxy-propylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-[1-(3-{3-[3-(2-Dimethylamino-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{3-[2-(4-Dimethylamino-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl]-piperidine-4-carboxylic acid amide;
1-{1-[3-(5-Methanesulfonyl-3-{3-[2-(4-methoxy-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-[4-trifluoromethyl-3-[2-(3-trifluoromethyl-piperidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(2-Hydroxymethyl-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-[4-trifluoromethyl-3-[2-(4-trifluoromethyl-piperidin-1-yl)-ethylsulfanyl]-phenyl}-4,5,6,7-tetrahydro-pyrazolo[4,3-c]pyridin-1-yl)-propyl]-piperidin-4-yl}-pyrrolidin-2-one;
1-[1-(3-[5-Methanesulfonyl-3-[3-(2-thiazolidin-3-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-(1-[3-[5-Methanesulfonyl-3-(3-[2-[(thiophen-2-ylmethyl)-amino]-ethylsulfanyl]-4-trifluoromethyl-phenyl)-4,5,6,7-tetrahydro-pyrazolo[4,3-c]pyridin-1-yl]-propyl}-piperidin-4-yl)-pyrrolidin-2-one;
1-{1-[3-(3-{3-[2-(3-Hydroxy-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,4-Dihydro-1H-isoquinolin-2-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-methyl-[1,4]diazepan-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(2-Cyclohex-1-enyl-ethylamino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-[3-[2-(2-phenoxy-ethylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-[3-[2-(2-morpholin-4-yl-ethylamino)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(4-phenethyl-piperazin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-(1-{3-[3-(3-{2-[(1-Ethyl-pyrrolidin-2-ylmethyl)-amino]-ethylsulfanyl}-4-trifluoromethyl-phenyl)-5-methanesulfonyl-4,5,6,7-tetrahydro-pyrazolo[4,3-c]pyridin-1-yl]-propyl}-piperidin-4-yl)-pyrrolidin-2-one;
1-{1-[3-(3-{3-[2-(Benzyl-methyl-amino)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-ethyl}-piperidine-3-carboxylic acid diethylamide;
1-{1-[3-(3-{3-[2-(3-Dimethylamino-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
5-Methanesulfonyl-3-[3-(2-piperidin-1-yl-ethylsulfanyl)-4-trifluoromethyl-phenyl]-1-(3-piperidin-1-yl-propyl)-4,5,6,7-tetrahydro-1H-pyrazolo[4,3-c]pyridine;
5-Methanesulfonyl-3-[3-(2-piperidin-1-yl-ethylsulfanyl)-4-trifluoromethyl-phenyl]-1-[3-(4-pyrrolidin-1-yl-piperidin-1-yl)-propyl]-4,5,6,7-tetrahydro-1H-pyrazolo[4,3-c]pyridine;
1-[4-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperazin-1-yl]-ethanone;
5-Methanesulfonyl-3-[3-(2-piperidin-1-yl-ethylsulfanyl)-4-trifluoromethyl-phenyl]-1-(3-pyrrolidin-1-yl-propyl)-4,5,6,7-tetrahydro-1H-pyrazolo[4,3-c]pyridine;
1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-pyrrolidin-3-ol;
5-Methanesulfonyl-1-(3-morpholin-4-yl-propyl)-3-[3-(2-piperidin-1-yl-ethylsulfanyl)-4-trifluoromethyl-phenyl]-4,5,6,7-tetrahydro-1H-pyrazolo[4,3-c]pyridine;
4-[1-(3-{5-Methanesulfonyl-3-[3-(2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-4H-benzo[1,4]oxazin-3-one;
{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-carbamic acid tert-butyl ester;
1-[1-(2-Hydroxy-3-{5-methanesulfonyl-3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one; N-Biphenyl-4-yl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-(2-phenoxy-ethyl)-acetamide;
1-{1-[2-Hydroxy-3-(5-methanesulfonyl-3-{3-[2-oxo-2-(4-phenyl-3,6-dihydro-2H-pyridin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-indan-2-yl-acetamide;
N-Biphenyl-3-yl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-(9H-Fluoren-1-yl)-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-(4-Benzyloxy-phenyl)-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
N-Benzyl-2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetamide;
2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-N-phenethyl-acetamide;
1-{1-[3-(3-{3-[2-(3-Hydroxy-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-pyrrolidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxymethyl-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Hydroxy-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(3-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-[3-[2-(octahydro-isoquinolin-2-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-(2-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Fluoro-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
(S)-1-{1-[3-(5-Methanesulfonyl-3-{3-[2-oxo-2-(2-trifluoromethyl-pyrrolidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
N-(4-Fluoro-phenyl)-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetamide;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-N,N-dimethyl-acetamide;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-N-methyl-acetamide;
1-{1-[3-(3-{3-[2-(4-Dimethylamino-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3-Dimethylamino-pyrrolidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[3-(2-[1,4′]Bipiperidinyl-1′-yl-2-oxo-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-methyl-piperidin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetyl}-piperidine-4-carboxylic acid amide;
1-[1-(3-{5-Methanesulfonyl-3-[3-(2-oxo-2-pyrrolidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-oxo-2-piperidin-1-yl-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{5-Methanesulfonyl-3-[3-(2-morpholin-4-yl-2-oxo-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
1-[1-(3-{3-[3-(2-Azepan-1-yl-2-oxo-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(5-Methanesulfonyl-3-{3-[2-(4-methyl-[1,4]diazepan-1-yl)-2-oxo-ethylsulfanyl]-4-trifluoromethyl-phenyl}-4,5,6,7-tetrahydro-pyrazolo[4,3c]pyridin-1-yl)-propyl]-piperidin-4-yl}-pyrrolidin-2-one;
1-{1-[3-(3-{3-[2-(3,6-Dihydro-2H-pyridin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{3-[2-(3,4-Dihydro-1H-isoquinolin-2-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethylphenylsulfanyl]-N-methyl-N-(1-methyl-piperidin-4-yl)-acetamide;
1-{1-[3-(5-Methanesulfonyl-3-{3-[2-oxo-2-(4-phenyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
N-Cyclohexyl-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethylphenylsulfanyl]-N-methyl-acetamide;
N-Benzyl-2-[5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetamide;
1-{1-[3-(5-Methanesulfonyl-3-{3-[2-(4-methyl-piperazin-1-yl)-2-oxo-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(3-{3-[2-(4-Benzyl-piperidin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-oxo-2-(4-phenethyl-piperidin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{1-[3-(5-Methanesulfonyl-3-{3-[2-oxo-2-(4-phenyl-3,6-dihydro-2H-pyridin-1-yl)-ethylsulfanyl]-4-trifluoromethyl-phenyl}-4,5,6,7-tetrahydro-pyrazolo[4,3-c]pyridin-1-yl)-propyl]-piperidin-4-yl}-pyrrolidin-2-one;
1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl phenylsulfanyl]-acetyl}-piperidine-4-carboxylic acid diethylamide;
(R)-1-{2-[5-(5-Methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-2-trifluoromethyl-phenylsulfanyl]-acetyl}-pyrrolidine-2-carboxylic acid amide;
1-{1-[3-(3-{3-[2-(4-Isopropyl-piperazin-1-yl)-2-oxo-ethylsulfanyl]-4-trifluoromethyl-phenyl}-5-methanesulfonyl-4,5,6,7-tetrahydro-pyrazolo[4,3-c]pyridin-1-yl)-propyl]-piperidin-4-yl}-pyrrolidin-2-one;
2-(5-{5-Methanesulfonyl-1-[3-(4-pyrrolidin-1-yl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone;
2-(5-{5-Methanesulfonyl-1-[3-(4-trifluoromethyl-piperidin-1-yl)-propyl]-4,5,6,7-tetrahydro-1H-pyrazolo[4,3-c]pyridin-3-yl}-2-trifluoromethyl-phenylsulfanyl)-1-morpholin-4-yl-ethanone;
2-(5-{1-[3-(4-Acetyl-piperazin-1-yl)-propyl]-5-methanesulfonyl-4,5,6,7-tetrahydro-1H-pyrazolo[4,3-c]pyridin-3-yl}-2-trifluoromethyl-phenylsulfanyl)-1-morpholin-4-yl-ethanone;
-(3-[5-Methanesulfonyl-3-[3-(2-morpholin-4-yl-2-oxo-ethylsulfanyl)-4-trifluoromethyl-phenyl]-4,5,6,7-tetrahydro-pyrazolo[4,3-c]pyridin-1-yl}-propyl)-piperidin-4-yl]-carbamic acid tert-butyl ester;
N-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
N-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-2-phenyl-acetamide;
4-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-piperidine-1-carboxylic acid tert-butyl ester; ({2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-methyl)-carbamic acid tert-butyl ester;
4-Fluoro-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
4-Hydroxy-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
4-Dimethylamino-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-benzamide;
2-(4-Fluoro-phenyl)-N-{2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-acetamide;
3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-piperidine-1-carboxylic acid tert-butyl ester;
3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-pyrrolidine-1-carboxylic acid tert-butyl ester;
(3-{2-[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethylcarbamoyl}-propyl)-carbamic acid tert-butyl ester;
Piperidine-3-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
Pyrrolidine-3-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
Piperidine-4-carboxylic acid {2-[5-(1-{2-hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-ethyl}-amide;
(2S)-1-(4,4-Dimethyl-piperidin-1-yl)-3-(3-{3-[2-(4-fluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-propan-2-ol; (2S)—N-{1-[3-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-acetamide; and
(2S)-1-(3-{3-[2-(3,3-Difluoro-piperidin-1-yl)-ethylsulfanyl]-4-trifluoromethyl-phenyl}-5-methanesulfonyl-4,5,6,7-tetrahydro-pyrazolo[4,3-c]pyridin-1-yl)-3-(4-methylpiperidin-1-yl)-propan-2-ol;
and pharmaceutically acceptable salts thereof.

78. A method according to claim 72, wherein said chemical entity is selected from the group consisting of:
1-[1-(3-{3-[3-(2-Amino-ethylsulfanyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-pyrrolidin-2-one;
1-[1-(3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
N-[1-(3-{3-[3-(2-Hydroxy-ethylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-acetamide;
5-Dimethylamino-3-[1-(3-{3-[3-(2-hydroxy-ethylsulfanyl)-4-trifluoromethyl-phenyl]-5-methanesulfonyl-4,5,6,7-tetrahydro-pyrazolo[4,3-c]pyridin-1-yl}-propyl)piperidin-4-yl]-1-methyl-1,3-dihydro-imidazo[4,5-b]pyridin-2-one;
[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid ethyl ester;
[5-(1-{2-Hydroxy-3-[4-(2-oxo-pyrrolidin-1-yl)-piperidin-1-yl]-propyl}-5-methanesulfonyl-4,5,6,7-tetrahydro-1H-pyrazolo[4,3-c]pyridin-3-yl)-2-trifluoromethyl-phenylsulfanyl]-acetic acid;
3-(3-Benzylsulfanyl-4-chloro-phenyl)-5-methanesulfonyl-1-(3-morpholin-4-yl-propyl)-4,5,6,7-tetrahydro-1H-pyrazolo[4,3-c]pyridine;
3-(4-Chloro-3-phenethylsulfanyl-phenyl)-5-methanesulfonyl-1-(3-morpholin-4-yl-propyl)-4,5,6,7-tetrahydro-1H-pyrazolo[4,3-c]pyridine;
5-Methanesulfonyl-1-(3-morpholin-4-yl-propyl)-3-[3-(2-phenoxy-ethylsulfanyl)-4-trifluoromethyl-phenyl]-4,5,6,7-tetrahydro-1H-pyrazolo[4,3-c]pyridine;
3-[4-Chloro-3-(furan-2-ylmethylsulfanyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-propylsulfanyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(2-phenoxy-ethylsulfanyl)-phenyl]-5-methanesulfonyl-1-(3-morpholin-4-yl-propyl)-4,5,6,7-tetrahydro-1H-pyrazolo[4,3-c]pyridine;
1-(3-{3-[3-(4-Chloro-benzylsulfanyl)-4-trifluoromethyl-phenyl]-5-methanesulfonyl-4,5,6,7-tetrahydro-pyrazolo[4,3-c]pyridin-1-yl}-propyl)-piperidin-4-yl]-pyrrolidin-2-one;
3-[4-Chloro-3-(4-chloro-benzylsulfanyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-fluoro-benzylsulfanyl)-phenyl]-5-methanesulfonyl-1-(3-morpholin-4-yl-propyl)-4,5,6,7-tetrahydro-1H-pyrazolo[4,3-c]pyridine;
1-[1-(3-{5-Methanesulfonyl-3-[3-(2-phenoxy-ethylsulfanyl)-4-trifluoromethyl-phenyl]-4,5,6,7-tetrahydro-pyrazolo[4,3-c]pyridin-1-yl}-propyl)-piperidin-4-yl]-pyrrolidin-2-one;
3-[4-Chloro-3-(2-pyrazin-2-yl-ethylsulfanyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-chloro-propylsulfanyl)-phenyl]-5-methanesulfonyl-1-(3-morpholin-4-yl-propyl)-4,5,6,7-tetrahydro-1H-pyrazolo[4,3-c]pyridine; and
3-(4-Chloro-3-phenylmethanesulfinyl-phenyl)-5-methanesulfonyl-1-(3-morpholin-4-yl-propyl)-4,5,6,7-tetrahydro-1H-pyrazolo[4,3-c]pyridine;
and pharmaceutically acceptable salts thereof.

79. A method according to claim 72, wherein said chemical entity is selected from the group consisting of:
1-[1-(3-{3-[3-({2-[(1,1-Dimethylethyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
3-[3-({2-[(3S)-3-Methylmorpholin-4-yl]ethyl]sulfanyl)-4-(trifluoromethyl)phenyl]-5-(methylsulfonyl)-1-(3-piperidin-1-ylpropyl)-4,5,6,7-tetrahydro-1H-pyrazolo[4,3-c]pyridine;
3-[3-({2-[(2R,6R)-2,6-Dimethylmorpholin-4-yl]ethyl]sulfanyl)-4-(trifluoromethyl)phenyl]-5-(methylsulfonyl)-1-(3-piperidin-1-ylpropyl)-4,5,6,7-tetrahydro-1H-pyrazolo[4,3-c]pyridine;
{4-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]morpholin-2-yl]methanol;
{(3S)-4-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]morpholin-3-yl]methanol;
5-(Dimethylamino)-1-methyl-3-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-imidazo[4,5-b]pyridin-2-one;
Ethyl(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
N-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl}sulfanyl)ethyl]propan-1-amine; N-[2-({5-[5-(Methylsulfonyl)-1-{3-[4-(piperidin-1-ylcarbonyl)piperidin-1-yl]propyl}-4,5,6,7-tetrahydro-1H-pyrazolo[4,3-c]pyridin-3-yl]-2-(trifluoromethyl)phenyl}sulfanyl)ethyl]propan-1-amine;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
5-(Dimethylamino)-3-(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1-methyl-1,3-dihydro-2H-imidazo[4,5-b]pyridin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
2-(4-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-(4-Cyclopropylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-morpholin-4-ylpropan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-pyrrolidin-1-ylpropan-2-ol;
1-[4-(Dimethylamino)piperidin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-(4-Methylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-({2-[2-(Hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-[1-(2-Hydroxy-3-{3-[3-({2-[2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
4-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)ethyl]-3-methylpiperazin-2-one; 4-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]-3-methylpiperazin-2-one;
4-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)-3-methylpiperazin-2-one;
1-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)ethyl]piperidin-4-ol;
1-[1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
2-[4-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperazin-1-yl]benzonitrile;
1-(2-{[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
5-(Dimethylamino)-3-[1-(2-hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-(4-Methylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4,4-Dimethylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4-Fluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(1,1-Dimethylethyl)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-morpholin-4-ylpropan-2-ol;
5-(Dimethylamino)-3-(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1-methyl-1,3-dihydro-2H-imidazo[4,5-b]pyridin-2-one;
1-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
2-(4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{5-(Ethylsulfonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl]sulfanyl)ethyl]piperidin-3-ol;
1-(2-{[5-{1-[2-Hydroxy-3-(4-hydroxy-4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidin-3-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(2-hydroxyethyl)piperidin-1-yl]propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxamide;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-2-hydroxyacetamide; 1-{1-[(2S)-2-Hydroxy-3-{3-[3-({2-[(2R)-2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[(2S)-2-Hydroxy-3-[5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]-4-methylpiperidin-4-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(octahydroisoquinolin-2(1H)-yl)propan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrazin-2-ylpiperazin-1-yl)propan-2-ol;
(2R)-1-[4-(3,5-Dichloropyridin-4-yl)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(3,6-Dihydropyridin-1(2H)-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-[4-(1,1-Dimethylethyl)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
N-{1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide; (2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1,1-dimethylethyl)piperidin-1-yl]propan-2-ol;
(2S)-1-(4-Cyclohexylpiperidin-1-yl)-3-{3-[3-{[2-(4,4-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(Hexahydrocyclopenta[c]pyrrol-2(1H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-methylpiperidin-4-ol;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidine-4-carboxamide;
(2S)-1-{3-[3-({2-[4-(1,1-Dimethylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
N-[1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-yl]acetamide;
2-Hydroxy-N-[1-(2-{[5-{1-[(2S)-2-hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-yl]acetamide;
1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)-4-pyridin-2-ylpiperidin-4-ol;
(2S)-1-{3-[3-({2-[(3S)-3-Methylmorpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol; (2S)-1-{3-[3-{[2-(Hexahydrocyclopenta[c]pyrrol-2(1H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)-4-methylpiperidin-4-ol;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl}ethyl)piperidine-4-carboxamide;
(2S)-1-{3-[3-({2-[4-(1-Hydroxy-1-methylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
5-(Dimethylamino)-3-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-1-methyl-1,3-dihydro-2H-imidazo[4,5-b]pyridin-2-one;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyrrolidin-1-ylpiperidin-1-yl)propan-2-ol;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-(1-{2-Hydroxy-3-[5-(phenylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-(1-{3-[5-(Ethylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-2-hydroxypropyl}piperidin-4-yl)pyrrolidin-2-one;
1-[5-(Ethylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-{1-[2-Hydroxy-3-(5-[(phenylmethyl)sulfonyl]-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-[5-(Hydroxyacetyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-[5-(Hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-(1-{2-Hydroxy-3-[5-(hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)piperidin-4-yl]pyrrolidin-2-one;
1-[3-{3-[(2-Morpholin-4-ylethyl)sulfanyl]-4-(trifluoromethyl)phenyl}-5-(piperidin-4-ylacetyl)-4,5,6,7-tetrahydro-1H-pyrazolo[4,3-c]pyridin-1-yl]-3-piperidin-1-ylpropan-2-ol;
Methyl[1-(2-hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl](oxo)acetate;
Methyl(1-{2-hydroxy-3-[4-(2-oxopyrrolidin-1-yl)piperidin-1-yl]propyl}-3-{3-[(2-piperidin-1-ylethyl)sulfanyl]-4-(trifluoromethyl)phenyl}-1,4,6,7-tetrahydro-5H-pyrazolo[4,3-c]pyridin-5-yl)(oxo)acetate;
1-(5-Acetyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-{1-[3-(5-Acetyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}pyrrolidin-2-one;
1-(1-{3-[5-(Furan-2-ylcarbonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-2-hydroxypropyl}piperidin-4-yl)pyrrolidin-2-one;
1-(5-Methyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
2-(1-{(2S)-3-[4-(Acetylamino)piperidin-1-yl]-2-hydroxypropyl}-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
2-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-2-oxoacetamide;
(2S)-1-{3-[3-{[2-(4-Fluoropiperidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-piperidin-1-ylpropan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-methyl-1,4,6,7-tetrahydro-5H-pyrazolo[4,3-c]pyridine-5-carboxamide;
5-(Methylsulfonyl)-3-{3-[(2-morpholin-4-yl-2-oxoethyl)sulfanyl]-4-(trifluoromethyl)phenyl]-1-(3-piperidin-1-ylpropyl)-4,5,6,7-tetrahydro-1H-pyrazolo[4,3-c]pyridine;
5-(Methylsulfonyl)-3-[3-[(2-morpholin-4-yl-2-oxoethyl)sulfanyl]-4-(trifluoromethyl)phenyl]-1-(3-morpholin-4-ylpropyl)-4,5,6,7-tetrahydro-1H-pyrazolo[4,3-c]pyridine;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol; 1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-morpholin-4-ylpiperidin-1-yl)propan-2-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyridin-2-ylpiperidin-1-yl)propan-2-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-{4-[2-(Methyloxy)phenyl]piperidin-1-yl}-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propan-2-ol;
1-{[1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-[4-(1,1-Dimethylethyl)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyridin-2-ylpiperidin-1-yl)propan-2-ol;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-{4-[2-(methyloxy)phenyl]piperidin-1-yl}propan-2-ol;
1-{3-[3-{[2-(4-Fluoropiperidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-(4-pyridin-2-ylpiperidin-1-yl)propan-2-ol;
1-(5-Acetyl-3-{3-[(2-morpholin-4-yl-2-oxoethyl)sulfanyl]-4-(trifluoromethyl)phenyl-4,5,6,7-tetrahydro-1H-pyrazolo[4,3-c]pyridin-1-yl)-3-{4-[2-(methyloxy)phenyl]piperidin-1-yl}propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(1,1-dimethylethyl)piperidin-1-yl]propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-methylpiperidin-1-yl)propan-2-ol;
1-{1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}pyrrolidin-2-one; and
1-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4-pyridin-2-ylpiperidin-1-yl)propan-2-ol;
and pharmaceutically acceptable salts thereof.

80. A method according to claim 72, wherein said chemical entity is selected from the group consisting of:
1-[3-{3-[2-(4-Fluoro-piperidin-1-yl)-ethylsulfanyl]-4-trifluoromethyl-phenyl}-1-(2-hydroxy-3-piperidin-1-yl-propyl)-1,4,6,7-tetrahydro-pyrazolo[4,3-c]pyridin-5-yl]-2-hydroxy-ethanone;
(2S)-1-(4-Cyclohexyl-piperidin-1-yl)-3-(3-{3-[2-((3S)-fluoro-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-propan-2-ol;
N-[1-(2-{5-[1-((2S)-Hydroxy-3-morpholin-4-yl-propyl)-5-methanesulfonyl-4,5,6,7-tetrahydro-1H-pyrazolo[4,3-c]pyridin-3-yl]-2-trifluoromethyl-phenylsulfanyl}-ethyl)-piperidin-4-yl]-acetamide; (2S)-1-(3-{3-[2-((3S)-Fluoro-pyrrolidin-1-yl)-ethylsulfanyl]-4-trifluoromethyl-phenyl}-5-methanesulfonyl-4,5,6,7-tetrahydro-pyrazolo[4,3-c]pyridin-1-yl)-3-(3′,4′,5′,6′-tetrahydro-2′H-[2,4′]bipyridinyl-1′-yl)-propan-2-ol;
(3S)-1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-3-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-pyrrolidin-1-ylpropan-2-ol;
1-Piperidin-1-yl-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]pyrrolidin-3-ol;
1-(3-{3-[(2-Morpholin-4-ylethyl)sulfanyl]-4-(trifluoromethyl)phenyl-4,5,6,7-tetrahydro-1H-pyrazolo[4,3-c]pyridin-1-yl)-3-piperidin-1-ylpropan-2-ol;
1-Morpholin-4-yl-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(4-Methylpiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-ol;
1-[2-({5-[1-(2-Hydroxy-3-piperidin-1-ylpropyl)-4,5,6,7-tetrahydro-1H-pyrazolo[4,3-c]pyridin-3-yl]-2-(trifluoromethyl)phenyl}sulfanyl)ethyl]piperidin-3-ol;
1-(4-Fluoropiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-morpholin-4-ylpropan-2-ol;
1-(Hexahydrocyclopenta[c]pyrrol-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol
1-(4,4-Dimethylpiperidin-1-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]-4-methylpiperidin-4-ol;
1-[4-(Methyloxy)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(4-Fluoropiperidin-1-yl)ethyl]sulfanyl}-4-(trifluoromethyl)phenyl]-5-methyl-4,5,6,7-tetrahydro-1H-pyrazolo[4,3-c]pyridin-1-yl}-3-piperidin-1-ylpropan-2-ol; (2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-methylpiperidin-1-yl)propan-2-ol;
1-(1,3-Dihydro-2H-isoindol-2-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
3-{3-[(2-Azetidin-1-ylethyl)sulfanyl]-4-(trifluoromethyl)phenyl}-5-(methylsulfonyl)-1-(3-piperidin-1-ylpropyl)-4,5,6,7-tetrahydro-1H-pyrazolo[4,3-c]pyridine;
1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidine-4-carboxamide;
1-(4-Acetylpiperazin-1-yl)-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidine-4-carboxamide;
1-(3,3-Difluoropyrrolidin-1-yl)-3-{3-[3-{[2-(3,3-difluoropyrrolidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-(4,4-Dimethylpiperidin-1-yl)-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
5-(Methylsulfonyl)-1-(3-piperidin-1-ylpropyl)-3-{3-[(2-pyrrolidin-1-ylethyl)sulfanyl]-4-(trifluoromethyl)phenyl}-4,5,6,7-tetrahydro-1H-pyrazolo[4,3-c]pyridine;
1-(3,4-Dihydroisoquinolin-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
N-Methyl-N-[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]propan-2-amine;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[(3S)-3-(trifluoromethyl)pyrrolidin-1-yl]propan-2-ol;
1-(Octahydroisoquinolin-2(1H)-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
2-(1-[(2S)-2-Hydroxy-3-piperidin-1-ylpropyl]-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
1-(1,4-Dioxa-8-azaspiro[4.5]dec-8-yl)-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[4-(1-Hydroxy-1-methylethyl)piperidin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}urea;
(2S)-1-{5-Acetyl-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide; 1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidine-4-carboxamide;
1-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]imidazolidin-4-one;
1-{3-[3-[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
5-(Methylsulfonyl)-3-{3-[(2-morpholin-4-ylethyl)sulfanyl]-4-(trifluoromethyl)phenyl}-1-(3-piperidin-1-ylpropyl)-4,5,6,7-tetrahydro-1H-pyrazolo[4,3-c]pyridine;
1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidine-4-carboxamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-pyrrolidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(hexahydrocyclopenta[c]pyrrol-2(1H)-yl)propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(4,4-dimethylpiperidin-1-yl)propan-2-ol;
1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidine-4-carboxamide;
2-Hydroxy-N-{1-[2-hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
2-Hydroxy-N-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
(2S)-1-[1-(2-Hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl]-1-oxopropan-2-ol;
1-(5-[(Methyloxy)acetyl]-3-{3-[(2-morpholin-4-ylethyl)sulfanyl]-4-(trifluoromethyl)phenyl]-4,5,6,7-tetrahydro-1H-pyrazolo[4,3-c]pyridin-1-yl)-3-piperidin-1-ylpropan-2-ol;
1-[2-({5-[5-(Hydroxyacetyl)-1-(2-hydroxy-3-piperidin-1-ylpropyl)-4,5,6,7-tetrahydro-1H-pyrazolo[4,3-c]pyridin-3-yl]-2-(trifluoromethyl)phenyl]sulfanyl)ethyl]piperidin-3-ol;
(2S)-1-{3-[3-{[2-(4-Fluoropiperidin-1-yl)ethyl]sulfanyl}-4-(trifluoromethyl)phenyl]-5-propanoyl-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-glycyl-4,5,6,7-tetrahydro-1H-pyrazolo[4,3-c]pyridin-1-yl}-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(1,3-dihydro-2H-isoindol-2-yl)propan-2-ol;
N-[2-({5-[5-(Methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]cyclohexanamine;
1-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}urea;
N-(2-{[5-{1-[3-(4-Cyclopropylpiperazin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)propan-1-amine;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-(4-pyridin-4-ylpiperidin-1-yl)propan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
1-(4-Methylpiperidin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(2S)-1-{3-[3-{[2-(5,6-Dihydropyrimidin-1(4H)-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(4-Methylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}pyrrolidin-3-ol;
1-[(3S)-3-(Hydroxymethyl)pyrrolidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[(3S)-3-Methylmorpholin-4-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]urea;
(2S)-1-(5,6-Dihydropyrimidin-1(4H)-yl)-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-morpholin-4-ylpropan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}pyrrolidin-3-ol;
5-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-2,4-dihydro-3H-1,2,4-triazol-3-one;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
2-{1-[(2S)-2-Hydroxy-3-piperidin-1-ylpropyl]-3-[3-{[2-(4-methylpiperidin-1-yl)ethyl]sulfanyl}-4-(trifluoromethyl)phenyl]-1,4,6,7-tetrahydro-5H-pyrazolo[4,3-c]pyridin-5-yl}-2-oxoacetamide;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-(4-pyrrolidin-1-ylpiperidin-1-yl)propan-2-ol;
1-(4-Morpholin-4-ylpiperidin-1-yl)-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
2-{1-(2-Hydroxy-3-piperidin-1-ylpropyl)-3-[3-{[2-(4-methylpiperidin-1-yl)ethyl]sulfanyl}-4-(trifluoromethyl)phenyl]-1,4,6,7-tetrahydro-5H-pyrazolo[4,3-c]pyridin-5-yl}-2-oxoacetamide;
N-{1-[(2S)-3-(5-Acetyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}acetamide;
(4R)-1-{1-[(2S)-3-(5-Acetyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}-4-hydroxypyrrolidin-2-one;
Ethyl {1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}carbamate;
N-[2-({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)ethyl]methanesulfonamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-dimethyl-1,4,6,7-tetrahydro-5H-pyrazolo[4,3-c]pyridine-5-carboxamide;
1-(5-[(Methylsulfanyl)acetyl]-3-{3-[(2-morpholin-4-ylethyl)sulfanyl]-4-(trifluoromethyl)phenyl}-4,5,6,7-tetrahydro-1H-pyrazolo[4,3-c]pyridin-1-yl)-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(3,4-dihydroisoquinolin-2(1H)-yl)propan-2-ol;
(2S)-1-{3-[3-{[2-(4-Fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-1-oxopropan-2-ol;
(2S)-1-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-1-oxopropan-2-ol;
1-{3-[3-{[2-(4-Fluoropiperidin-1-yl)ethyl]sulfanyl}-4-(trifluoromethyl)phenyl]-5-[(methyloxy)acetyl]-4,5,6,7-tetrahydro-1H-pyrazolo[4,3-c]pyridin-1-yl}-3-piperidin-1-ylpropan-2-ol; (2S)-1-{3-[3-{[2-(4-Fluoropiperidin-1-yl)ethyl]sulfanyl}-4-(trifluoromethyl)phenyl]-5-[(methyloxy)acetyl]-4,5,6,7-tetrahydro-1H-pyrazolo[4,3-c]pyridin-1-yl}-3-piperidin-1-ylpropan-2-ol;
3-Methyl-4-[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)ethyl]piperazin-2-one;
N-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}-2-hydroxyacetamide;
1-[5-(Methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]-3-piperidin-1-ylpropan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(4,4-Dimethylpiperidin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
2-{4-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
N-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
1-(4-Methyl-1,4-diazepan-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
3-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
4-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
2-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenol;
1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}-4-methylpiperidin-4-ol;
(2S)-1-(3,6-Dihydropyridin-1(2H)-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
(3S)-1-[({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)acetyl]pyrrolidin-3-ol;
(3R)-1-[({5-[1-(2-Hydroxy-3-piperidin-1-ylpropyl)-5-(methylsulfonyl)-4,5,6,7-tetrahydro-1H-pyrazolo[4,3-c]pyridin-3-yl]-2-(trifluoromethyl)phenyl]sulfanyl)acetyl]pyrrolidin-3-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(2S)-1-{3-[3-({2-[(3S)-3-Methylmorpholin-4-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
(3S)-1-(2-{[5-{1-[(2S)-2-Hydroxy-3-piperidin-1-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-3-ol;
(2S)-1-(5,6-Dihydropyrimidin-1(4H)-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(5,6-Dihydropyrimidin-1(4H)-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-(4-Methylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(4-Fluorophenyl)piperazin-1-yl]-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
N-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]-2-hydroxyacetamide;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-4-ylpiperidin-1-yl)propan-2-ol;
1-[4-(2-Fluorophenyl)piperazin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
1-[4-(4-Fluorophenyl)piperazin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-Azepan-1-yl-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
(3R)-1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-3-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-ol; (3S)-1-{2-Hydroxy-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}pyrrolidin-3-ol;
(3R)-1-[2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}pyrrolidin-3-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
1-(3-{3-[(2-Oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
(3S)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-ol;
N-Methyl-2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)-N-(phenylmethyl)ethanamine;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperidin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-phenylpiperazin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-(4-pyridin-2-ylpiperidin-1-yl)propan-2-ol;
1-[1-(2-Hydroxy-3-{3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
(4R)-4-Hydroxy-1-{1-[(2S)-2-hydroxy-3-(5-methyl-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}pyrrolidin-2-one;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(1-hydroxy-1-methylethyl)piperidin-1-yl]propan-2-ol;
1-(5-Acetyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-(1,4-dioxa-8-azaspiro[4.5]dec-8-yl)propan-2-ol;
(2S)-1-{5-(Cyclobutylcarbonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
4-{1-[2-Hydroxy-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
3-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
4-{1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}benzonitrile;
1-{5-[(1-Aminocyclopropyl)carbonyl]-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}pyrrolidin-2-one;
N-(2-{[5-{1′-[2-Hydroxy-3-(4-methylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)methanesulfonamide;
2-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}benzonitrile;
2-{3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}-N-methyl-2-oxoacetamide;
1-{5-(N,N-Dimethylglycyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-(1-methylethyl)-1,4,6,7-tetrahydro-5H-pyrazolo[4,3-c]pyridine-5-carboxamide;
Methyl {3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}(oxo)acetate;
Methyl {3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridin-5-yl}(oxo)acetate;
1-{3-[3-{[2-(4-Fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-methyl-1-oxopropan-2-ol;
1-(4-Cyclopropylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol; 1-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]pyrrolidin-2-one;
N-(2-{[5-{5-(Methylsulfonyl)-1-[3-(4-pyrrolidin-1-ylpiperidin-1-yl)propyl]-4,5,6,7-tetrahydro-1H-pyrazolo[4,3-c]pyridin-3-yl}-2-(trifluoromethyl)phenyl]sulfanyl]ethyl)propan-1-amine;
(2S)-1-{3-[3-{[2-(4-Fluoropiperidin-1-yl)ethyl]sulfanyl}-4-(trifluoromethyl)phenyl]-5-[(methylsulfanyl)acetyl]-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-[(methylsulfanyl)acetyl]-4,5,6,7-tetrahydro-1H-pyrazolo[4,3-c]pyridin-1-yl}-3-piperidin-1-ylpropan-2-ol;
1-{4-[2-(Methyloxy)phenyl]piperidin-1-yl}-3-(3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-(4-Methylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-Azepan-1-yl-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{4-[4-(Methyloxy)phenyl]piperidin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[2-(Methyloxy)phenyl]piperidin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-pyrrolidin-1-ylpropan-2-ol;
1-(4-Acetylpiperazin-1-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}pyrrolidin-3-yl)acetamide;
1-{4-[3-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[4-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-{4-[2-(Methyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}praline;
(2S)-1-[(3S)-3-Methylmorpholin-4-yl]-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
1-(3,3-Dimethylpiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-3-methylpiperazin-2-one;
N-{1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperidin-4-yl}methanesulfonamide;
1-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]-4-pyridin-2-ylpiperidin-4-ol;
1-(2-{[5-{1-[2-Hydroxy-3-(4-hydroxy-4-pyridin-2-ylpiperidin-1-yl)propyl]-4,5,6,7-tetrahydro-1H-pyrazolo[4,3-c]pyridin-3-yl}-2-(trifluoromethyl)phenyl]sulfanyl]ethyl)piperidin-3-ol;
N-[1-(2-Hydroxy-3-{3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
(2S)-1-(4,4-Dimethylpiperidin-1-yl)-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
1-[(3S)-3-(Hydroxymethyl)pyrrolidin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-[(3S)-3-Methylmorpholin-4-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
(2S)-1-{3-[3-{[2-(4-Methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
1-[4-(3-Chlorophenyl)piperidin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-[4-(Methyloxy)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-Azepan-1-yl-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol; (2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-Azepan-1-yl-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
1-(2-{[5-(1-{2-Hydroxy-3-[4-(pyrimidin-2-yloxy)piperidin-1-yl]propyl}-4,5,6,7-tetrahydro-1H-pyrazolo[4,3-c]pyridin-3-yl)-2-(trifluoromethyl)phenyl]sulfanyl]ethyl)piperidin-3-ol;
1-(4-Aminopiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
N-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}methanesulfonamide;
2-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}phenol;
3-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}phenol;
1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-ol;
(3S)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-3-ol;
1-[2-(Hydroxymethyl)morpholin-4-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-3-ol;
1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol; (2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(3,6-dihydropyridin-1(2H)-yl)propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-3-ol;
(3R)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-3-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-ol;
N-Methyl-2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl]sulfanyl)-N-(2-phenylethyl)ethanamine;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[(2S)-2-(hydroxymethyl)pyrrolidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(5,6-dihydropyrimidin-1(4H)-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(5,6-dihydropyrimidin-1(4H)-yl)propan-2-ol;
(2S)-1-{3-[3-{[2-(4-Fluoropiperidin-1-yl)ethyl]sulfanyl}-4-(trifluoromethyl)phenyl]-5-(1H-1,2,4-triazol-3-ylcarbonyl)-4,5,6,7-tetrahydro-1H-pyrazolo[4,3-c]pyridin-1-yl}-3-piperidin-1-ylpropan-2-ol;
1-(3-{3-[(2-Morpholin-4-yl-2-oxoethyl)sulfanyl]-4-(trifluoromethyl)phenyl}-4,5,6,7-tetrahydro-1H-pyrazolo[4,3-c]pyridin-1-yl)-3-[4-(pyrrolidin-1-ylcarbonyl)piperidin-1-yl]propan-2-ol;
1-[4-(Morpholin-4-ylcarbonyl)piperidin-1-yl]-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(2-{[5-(1-{2-Hydroxy-3-[4-(pyrrolidin-1-ylcarbonyl)piperidin-1-yl]propyl}-4,5,6,7-tetrahydro-1H-pyrazolo[4,3-c]pyridin-3-yl)-2-(trifluoromethyl)phenyl]sulfanyl]ethyl)piperidin-3-ol;
(2S)-1-[4-(2-Fluorophenyl)piperazin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
(2S)-1-[4-(4-Fluorophenyl)piperazin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol; (2S)-1-(3-Fluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-(4-Fluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(4-Fluoropiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
N-[1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperidin-4-yl]methanesulfonamide;
4-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]phenol;
1-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)-4-pyridin-2-ylpiperidin-4-ol;
1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
3-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]phenol;
2-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]pyridine-3-carbonitrile;
(2S)-1-{3-[3-{[2-(4-Fluoropiperidin-1-yl)ethyl]sulfanyl}-4-(trifluoromethyl)phenyl]-5-(pyrrolidin-1-ylcarbonyl)-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-morpholin-4-ylpropan-2-ol;
N-(2-{[5-{1-[3-(4,4-Dimethylpiperidin-1-yl)-2-hydroxypropyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-(4,4-Difluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(3,3-Difluoropiperidin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
1-{3-[3-{[2-(4-Fluoropiperidin-1-yl)ethyl]sulfanyl}-4-(trifluoromethyl)phenyl]-5-[(2R)-tetrahydrofuran-2-ylcarbonyl]-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(4-fluorophenyl)piperazin-1-yl]propan-2-ol;
N-(1,1-Dimethylethyl)-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-(1-methylpropyl)-1,4,6,7-tetrahydro-5H-pyrazolo[4,3-c]pyridine-5-carboxamide;
1-[1-(3-{3-[3-{[2-(Cyclopropylamino)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
N,N-Diethyl-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
2-{4-[2-Hydroxy-3-(3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl)propyl]piperazin-1-yl}benzonitrile;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(2,4,6,7-tetrahydro-5H-pyrazolo[4,3-c]pyridin-5-yl)propan-2-ol; 1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(1,4,6,7-tetrahydro-5H-imidazo[4,5-c]pyridin-5-yl)propan-2-ol;
N-(1-{3-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
1-[1-(3-{3-[3-({2-[(1-Methylethyl)amino]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-{[2-(propylamino)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrrolidin-1-ylcarbonyl)piperidin-1-yl]propan-2-ol;
4-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}benzonitrile;
1-(4-{4-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperazin-1-yl}phenyl)ethanone;
N,N-Dimethyl-1-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-3-yl)methanamine;
3-{1-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperidin-4-yl}benzonitrile;
2-[4-(3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]benzonitrile;
2-{4-[(2S)-3-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl]piperazin-1-yl}benzonitrile;
1-[4-(1,1-Dimethylethyl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-(4,4-Dimethylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{4-[2-(Methyloxy)phenyl]piperidin-1-yl}-3-(5-methyl-3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
1-(4-Cyclopropylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-(4-Acetylpiperazin-1-yl)-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
2-{4-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperazin-1-yl}phenol;
1-[4-(1-Methylethyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{4-[2-(Ethyloxy)phenyl]piperazin-1-yl}-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propan-2-ol;
(2S)-1-{5-(Cyclopropylsulfonyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[4-(1-Hydroxy-1-methylethyl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]-4-methylpiperidin-4-ol;
(2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
N-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}pyrrolidin-3-yl)acetamide;
1-(4-Acetylpiperazin-1-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(Dimethylamino)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(1-Methylethyl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidine-4-carboxamide;
1-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-3-[4-(4-fluorophenyl)piperazin-1-yl]propan-2-ol;
(2S)-1-{3-[3-{[2-(4-Fluoropiperidin-1-yl)ethyl]sulfanyl}-4-(trifluoromethyl)phenyl]-5-(propylsulfonyl)-4,5,6,7-tetrahydro-1H-pyrazolo[4,3-c]pyridin-1-yl}-3-piperidin-1-ylpropan-2-ol
(2S)-1-{3-[3-{[2-(4-Fluoropiperidin-1-yl)ethyl]sulfanyl}-4-(trifluoromethyl)phenyl]-5-[(1-methylethyl)sulfonyl]-4,5,6,7-tetrahydro-1H-pyrazolo[4,3-c]pyridin-1-yl}-3-piperidin-1-ylpropan-2-ol; (2S)-1-(4,4-Dimethylpiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
2-[4-(3-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-2-hydroxypropyl)piperazin-1-yl]benzonitrile;
(2R)-1-(4,4-Dimethylpiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-(4,4-Dimethylpiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(methyloxy)phenyl]piperidin-1-yl}propan-2-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[4-(methyloxy)phenyl]piperidin-1-yl}propan-2-ol;
1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-dimethyl-1,4,6,7-tetrahydro-5H-pyrazolo[4,3-c]pyridine-5-sulfonamide;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(methyloxy)phenyl]piperazin-1-yl}propan-2-ol;
1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4-Fluoropiperidin-1-yl)ethyl]sulfanyl}-4-(trifluoromethyl)phenyl]-5-[(1-methyl-1H-imidazol-2-yl)carbonyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(hydroxymethyl)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(hydroxymethyl)piperidin-1-yl]propan-2-ol;
1-(5-Acetyl-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl)-3-(4-pyrrolidin-1-ylpiperidin-1-yl)propan-2-ol;
1-(3,4-Dihydroisoquinolin-2(1H)-yl)-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-Azepan-1-yl-3-{3-[3-{[2-(3,3-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-Azepan-1-yl-3-{3-[3-{[2-(4,4-difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-methylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(2-thienylcarbonyl)-4,5,6,7-tetrahydro-1H-pyrazolo[4,3-c]pyridin-1-yl}-3-piperidin-1-ylpropan-2-ol;
1-{3-[3-{[2-(4-Fluoropiperidin-1-yl)ethyl]sulfanyl}-4-(trifluoromethyl)phenyl]-5-(2-thienylcarbonyl)-4,5,6,7-tetrahydro-1H-pyrazolo[4,3-c]pyridin-1-yl}-3-piperidin-1-ylpropan-2-ol;
(2S)-1-[4-(3-Chlorophenyl)piperidin-1-yl]-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
N-[1-(3-{5-(Methylsulfonyl)-3-[3-{[2-(propylamino)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[3-(methyloxy)phenyl]piperazin-1-yl}propan-2-ol;
1-{3-[3-{[2-(4-Fluoropiperidin-1-yl)ethyl]sulfanyl}-4-(trifluoromethyl)phenyl]-5-(1-methyl-L-prolyl)-4,5,6,7-tetrahydro-1H-pyrazolo[4,3-c]pyridin-1-yl}-3-piperidin-1-ylpropan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[4-(methyloxy)phenyl]piperazin-1-yl}propan-2-ol;
1-{3-[3-{[2-(4-Fluoropiperidin-1-yl)ethyl]sulfanyl}-4-(trifluoromethyl)phenyl]-5-(piperidin-3-ylcarbonyl)-4,5,6,7-tetrahydro-1H-pyrazolo[4,3-c]pyridin-1-yl}-3-piperidin-1-ylpropan-2-ol;
4-{1-[3-(5-Ethyl-3-{3-[(2-oxo-2-pyrrolidin-1-ylethyl)sulfanyl]-4-(trifluoromethyl)phenyl}-4,5,6,7-tetrahydro-1H-pyrazolo[4,3-c]pyridin-1-yl)-2-hydroxypropyl]piperidin-4-yl}benzonitrile;
2-Hydroxy-N-{1-[2-hydroxy-3-(5-[(methyloxy)acetyl]-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
(2S)-1-{3-[3-{[2-(4-Fluoropiperidin-1-yl)ethyl]sulfanyl}-4-(trifluoromethyl)phenyl]-5-(morpholin-4-ylcarbonyl)-4,5,6,7-tetrahydro-1H-pyrazolo[4,3-c]pyridin-1-yl}-3-piperidin-1-ylpropan-2-ol; (2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-thiomorpholin-4-ylpropan-2-ol;
(2S)-1-(4,4-Difluoropiperidin-1-yl)-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
(2S)-1-{5-(Methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[1-(3-{3-[3-({2-[(2-Methylpropyl)amino]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-pyrrolidin-1-ylpropan-2-ol;
(2S)-1-{5-(Methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}-3-morpholin-4-ylpropan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(octahydroisoquinolin-2(1H)-yl)propan-2-ol;
N-[(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methyl]acetamide;
1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidine-4-carboxamide;
1-[1-(3-{3-[3-({2-[(3-Hydroxypropyl)amino]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[5-(Hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
1-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)urea;
1-(1,5-Dioxa-9-azaspiro[5.5]undec-9-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
(2S)-1-{3-[3-({2-[4-(1-Hydroxy-1-methylethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-piperidin-1-ylpropan-2-ol;
2-Hydroxy-N-(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-phenyl-1,4,6,7-tetrahydro-5H-pyrazolo[4,3-c]pyridine-5-carboxamide;
(2S)-1-[4-(1,1-Dimethylethyl)piperidin-1-yl]-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
N-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
(2S)-1-[4-(Dimethylamino)piperidin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
(2S)-1-{4-[2-(Ethyloxy)phenyl]piperazin-1-yl}-3-{3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}propan-2-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxylic acid;
(3S)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-3-carboxylic acid;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
(3R)-1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-3-carboxylic acid; (3R)-1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-3-carboxylic acid;
(2S)-1-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-phenylpiperidin-1-yl)propan-2-ol;
1-[(2S)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-3-carboxylic acid;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(hexahydrocyclopenta[c]pyrrol-2(1H)-yl)propan-2-ol;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-yl]acetamide;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[2-(2-hydroxyethyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-pyridin-2-ylpiperidin-1-yl)propan-2-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4,4-dimethylpiperidin-1-yl)propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4,4-dimethylpiperidin-1-yl)propan-2-ol;
N-[1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)pyrrolidin-3-yl]acetamide;
1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidine-4-carboxamide;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N-2-thienyl-1,4,6,7-tetrahydro-5H-pyrazolo[4,3-c]pyridine-5-carboxamide;
1-{3-[3-{[2-(4-Fluoropiperidin-1-yl)ethyl]sulfanyl}-4-(trifluoromethyl)phenyl]-5-(piperidin-4-ylacetyl)-4,5,6,7-tetrahydro-1H-pyrazolo[4,3-c]pyridin-1-yl}-3-piperidin-1-ylpropan-2-ol; 1-[5-(Hydroxyacetyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol;
1-{3-[3-{[2-(3,3-Difluoropyrrolidin-1-yl)ethyl]sulfanyl}-4-(trifluoromethyl)phenyl]-4,5,6,7-tetrahydro-1H-pyrazolo[4,3-c]pyridin-1-yl}-3-{4-[2-(ethyloxy)phenyl]piperazin-1-yl}propan-2-ol;
N-Cyclohexyl-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-methylpiperidin-4-ol;
(2S)-1-{3-[3-{[2-(4-Fluoropiperidin-1-yl)ethyl]sulfanyl}-4-(trifluoromethyl)phenyl]-5-[(4-methylpiperazin-1-yl)carbonyl]-4,5,6,7-tetrahydro-1H-pyrazolo[4,3-c]pyridin-1-yl}-3-piperidin-1-ylpropan-2-ol;
1-(3-{3-[(2-Piperidin-1-ylethyl)sulfanyl]-4-(trifluoromethyl)phenyl}-4,5,6,7-tetrahydro-1H-pyrazolo[4,3-c]pyridin-1-yl)-3-{4-[3-(trifluoromethyl)phenyl]piperazin-1-yl}propan-2-ol;
3-[3-{[2-(4-Fluoropiperidin-1-yl)ethyl]sulfanyl}-4-(trifluoromethyl)phenyl]-1-[(2S)-2-hydroxy-3-piperidin-1-ylpropyl]-N,N-bis(1-methylethyl)-1,4,6,7-tetrahydro-5H-pyrazolo[4,3-c]pyridine-5-carboxamide;
(2S)-1-[4-(1,1-Difluoroethyl)piperidin-1-yl]-3-{3-[3-({2-[(3S)-3-fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propan-2-ol;
(2S)-1-(4-Methylpiperidin-1-yl)-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propan-2-ol;
2-Hydroxy-N-{1-[2-hydroxy-3-(5-[(methylsulfanyl)acetyl]-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}acetamide;
1-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-(1,4′-Bipiperidin-1′-yl)-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol; 1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(trifluoromethyl)piperidin-1-yl]propan-2-ol;
(2S)-1-[4-(1,1-Dimethylethyl)piperidin-1-yl]-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyrrolidin-1-ylpiperidin-1-yl)propan-2-ol;
N-{1-[(2S)-2-Hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}acetamide;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
(2S)-1-{3-[3-{[2-(4-Fluoropiperidin-1-yl)ethyl]sulfanyl}-4-(trifluoromethyl)phenyl]-5-[(trifluoromethyl)sulfonyl]-4,5,6,7-tetrahydro-1H-pyrazolo[4,3-c]pyridin-1-yl}-3-piperidin-1-ylpropan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(octahydroquinolin-1(2H)-yl)propan-2-ol;
(2S)-1-[4-(1-Hydroxy-1-methylethyl)piperidin-1-yl]-3-{3-[3-{[2-(4-methylpiperidin-1-yl)ethyl]sulfanyl-4-(trifluoromethyl)phenyl]-5-(methylsulfonyl)-4,5,6,7-tetrahydro-1H-pyrazolo[4,3-c]pyridin-1-yl}propan-2-ol;
N-[(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)methyl]acetamide;
Ethyl(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
(2S)-1-[4-(1,1-Dimethylethyl)piperidin-1-yl]-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-{3-[3-{[2-(4-Fluoropiperidin-1-yl)ethyl]sulfanyl}-4-(trifluoromethyl)phenyl]-5-[(2S)-2-hydroxy-2-phenylacetyl]-4,5,6,7-tetrahydro-1H-pyrazolo[4,3-c]pyridin-1-yl}-3-piperidin-1-ylpropan-2-ol;
2-Hydroxy-N-[1-(2-{[5-{1-[(2S)-2-hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-yl]acetamide;
N-{1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide; (2S)-1-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
2-Hydroxy-N-(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
N-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-phenylpiperazin-1-yl)propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-pyridin-4-ylpiperidin-1-yl)propan-2-ol;
1-(5-Acetyl-3-{3-[(2-morpholin-4-yl-2-oxoethyl)sulfanyl]-4-(trifluoromethyl)phenyl-4,5,6,7-tetrahydro-1H-pyrazolo[4,3-c]pyridin-1-yl)-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-phenylpiperidin-1-yl)propan-2-ol;
2-(1-[(2S)-2-Hydroxy-3-{4-[(4R)-4-hydroxy-2-oxopyrrolidin-1-yl]piperidin-1-yl}propyl]-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl)-2-oxoacetamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyridin-4-ylpiperidin-1-yl)propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-phenylpiperazin-1-yl)propan-2-ol;
N-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
1-[(2S)-3-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidine-4-carboxamide;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
1-[1-(2-Hydroxy-3-{5-(hydroxyacetyl)-3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
1-{5-(1-Acetyl-L-prolyl)-3-[3-{[2-(4-fluoropiperidin-1-yl)ethyl]sulfanyl}-4-(trifluoromethyl)phenyl]-4,5,6,7-tetrahydro-1H-pyrazolo[4,3-c]pyridin-1-yl}-3-piperidin-1-ylpropan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-pyrimidin-2-ylpiperazin-1-yl)propan-2-ol;
1-[1-(3-[3-[3-{[2-(Cyclohexylamino)ethyl]sulfanyl]-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[2-Hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]-4-[3-(trifluoromethyl)phenyl]piperidin-4-ol;
2-(4-{2-Hydroxy-3-[5-(hydroxyacetyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
5-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-Hydroxy-1-(1-{3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[4-(3-Methyl-1,2,4-oxadiazol-5-yl)piperidin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-cyclopropylpiperazin-1-yl)propan-2-ol;
5-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-Hydroxy-1-(1-{2-hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyrrolidin-1-ylpiperidin-1-yl)propan-2-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
(2S)-1-{3-[3-{[2-(4-Fluoropiperidin-1-yl)ethyl]sulfanyl}-4-(trifluoromethyl)phenyl]-4,5,6,7-tetrahydro-1H-pyrazolo[4,3-c]pyridin-1-yl}-3-{4-[3-(trifluoromethyl)phenyl]piperazin-1-yl}propan-2-ol;
N-(2-{[5-{1′-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-(4-morpholin-4-ylpiperidin-1-yl)propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
Ethyl(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
N-(1-[2-Hydroxy-3-[5-(hydroxyacetyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
2-Hydroxy-N-[1-(2-hydroxy-3-{3-[3-({2-[(3S)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
2-Hydroxy-N-(1-[2-hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)acetamide;
2-Hydroxy-N-[1-(2-hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
Ethyl(1-{2-hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)carbamate;
2-Hydroxy-N-[1-(2-hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]acetamide;
N-[1-(2-Hydroxy-3-{5-(hydroxyacetyl)-3-[3-{[2-(3-hydroxypiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]methanesulfonamide;
(2S)-1-(1,5-Dioxa-9-azaspiro[5.5]undec-9-yl)-3-{3-[3-[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[4-(2-Methylphenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{4-[2-(Methyloxy)phenyl]piperidin-1-yl}-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(phenylmethyl)piperidin-1-yl]propan-2-ol;
N-{1-[(2S)-3-{3-[3-{[2-(3-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
1-(2-{[5-{1-[2-Hydroxy-3-(4-phenylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1,1-dimethylethyl)piperidin-1-yl]propan-2-ol;
1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
(3S)-1-({[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]acetyl)pyrrolidin-3-ol;
N-(1-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide; N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-(2-{[5-{1-[2-Hydroxy-3-(4-phenylpiperazin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)piperidin-4-ol;
(3R)-1-({[5-{1-[2-Hydroxy-3-(4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]acetyl)pyrrolidin-3-ol;
1-[5-(Methylsulfonyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-(4-pyridin-4-ylpiperidin-1-yl)propan-2-ol;
N-(1-{2-Hydroxy-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)ethanesulfonamide;
1-(3-{3-[3-[2-(Cyclopentylamino)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyridin-2-yloxy)piperidin-1-yl]propan-2-ol;
2-(4-{2-Hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)phenol;
1-(1-{3-[3-{3-[(2-{[2-(1H-Imidazol-4-yl)ethyl]amino]ethyl)sulfanyl]-4-(trifluoromethyl)phenyl}-5-(methylsulfonyl)-4,5,6,7-tetrahydro-1H-pyrazolo[4,3-c]pyridin-1-yl]propyl}piperidin-4-yl)pyrrolidin-2-one;
N-{1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}acetamide;
1-[5-(Hydroxyacetyl)-3-{3-[(2-morpholin-4-yl-2-oxoethyl)sulfanyl]-4-(trifluoromethyl)phenyl]-4,5,6,7-tetrahydro-1H-pyrazolo[4,3-c]pyridin-1-yl]-3-[4-(pyrrolidin-1-ylcarbonyl)piperidin-1-yl]propan-2-ol;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-phenylpiperidin-4-ol;
(2S)-1-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol;
(2S)-1-{3-[3-{[2-(3,3-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(1-hydroxy-1-methylethyl)piperidin-1-yl]propan-2-ol; 1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-pyridin-2-ylpiperidin-4-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
N-[1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)piperidin-4-yl]methanesulfonamide;
1-(2-{[5-{1-[(2S)-2-Hydroxy-3-morpholin-4-ylpropyl]-5-(methylsulfonyl)-4,5,6,7-tetrahydro-1H-pyrazolo[4,3-c]pyridin-3-yl}-2-(trifluoromethyl)phenyl]sulfanyl]ethyl)-4-pyridin-2-ylpiperidin-4-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
1-(3-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-2-hydroxypropyl)-4-pyridin-2-ylpiperidin-4-ol;
N-(1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyridin-2-yloxy)piperidin-1-yl]propan-2-ol;
1-[4-(2-Fluorophenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
(2S)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(2-pyridin-3-ylpiperidin-1-yl)propan-2-ol;
2-(4-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}piperazin-1-yl)phenol;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
1-[1-(3-{3-[3-{[2-(3,3-Dimethylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(3,3-Difluoropyrrolidin-1-yl)ethyl]sulfanyl}-4-(trifluoromethyl)phenyl]-5-(hydroxyacetyl)-4,5,6,7-tetrahydro-1H-pyrazolo[4,3-c]pyridin-1-yl}-3-[4-(pyrimidin-2-yloxy)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyrimidin-2-yloxy)piperidin-1-yl]propan-2-ol;
(2S)-1-(4-Cyclohexylpiperidin-1-yl)-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[4-(2-Fluorophenyl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[4-(3-Chloropyridin-2-yl)piperidin-1-yl]-3-[5-(methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]propan-2-ol;
N-Cyclopentyl-2-({5-[1-{2-hydroxy-3-[4-(5-oxo-4,5-dihydro-1H-1,2,4-triazol-3-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1,1-Dimethylethyl(2-{3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-oxoethyl)carbamate;
1-[4-(3-Methyl-1,2,4-oxadiazol-5-yl)piperidin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
2-(4-{2-Hydroxy-3-[5-(hydroxyacetyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
4-(2-{[5-{1-[2-Hydroxy-3-(4-pyrrolidin-1-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}ethyl)-3-methylpiperazin-2-one;
5-(1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
4-(1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2,4-dihydro-3H-1,2,4-triazol-3-one;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(pyrrolidin-1-ylcarbonyl)piperidin-1-yl]propan-2-ol;
1-[5-(Methylsulfonyl)-3-{3-[(2-pyrrolidin-1-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
N-Cyclopentyl-2-{[5-{1-[2-hydroxy-3-(4-morpholin-4-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl}acetamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
1,1-Dimethylethyl 4-{[2-({5-[5-(methylsulfonyl)-1-(3-piperidin-1-ylpropyl)-4,5,6,7-tetrahydro-1H-pyrazolo[4,3-c]pyridin-3-yl]-2-(trifluoromethyl)phenyl-sulfanyl)ethyl]amino]piperidine-1-carboxylate;
(2S)-1-(4-Cyclohexylpiperidin-1-yl)-3-{3-[3-{[2-(3-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
5-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-2,4-dihydro-3H-1,2,4-triazol-3-one;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrazin-2-ylpiperazin-1-yl)propan-2-ol;
1-{3-[3-({2-[2-(Hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
(2S)-2-Hydroxy-N-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}propanamide;
5-Chloro-3-{1-[2-hydroxy-3-(3-{3-[(2-piperidin-1-ylethyl)sulfanyl]-4-(trifluoromethyl)phenyl}-4,5,6,7-tetrahydro-1H-pyrazolo[4,3-c]pyridin-1-yl)propyl]piperidin-4-yl}-1-methyl-1,3-dihydro-2H-benzimidazol-2-one;
N-[4-(Dimethylamino)phenyl]-3-[3-{[2-(4-fluoropiperidin-1-yl)ethyl]sulfanyl}-4-(trifluoromethyl)phenyl]-1-[(2S)-2-hydroxy-3-piperidin-1-ylpropyl]-1,4,6,7-tetrahydro-5H-pyrazolo[4,3-c]pyridine-5-carboxamide;
(4R)-1-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-4-hydroxypyrrolidin-2-one;
1-{2-Hydroxy-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propyl}-4-phenylpiperidine-4-carbonitrile;
(4S)-1-{1-[(2S)-3-{3-[3-({2-[(3S)-3-Fluoropyrrolidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-4-hydroxypyrrolidin-2-one;
1-[1-(3-{5-(Methylsulfonyl)-3-[3-({2-[(2-phenylethyl)amino]ethyl]sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
N-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[1-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]pyrrolidin-2-one;
1-{4-[2-(Methyloxy)phenyl]piperidin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]ethanesulfonamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
Phenylmethyl(2-{[5-{5-acetyl-1-[2-hydroxy-3-(4-pyridin-2-ylpiperidin-1-yl)propyl]-4,5,6,7-tetrahydro-1H-pyrazolo[4,3-c]pyridin-3-yl}-2-(trifluoromethyl)phenyl]sulfanyl}ethyl)carbamate;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
1-{4-[2-(Methyloxy)phenyl]piperazin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-pyridin-2-ylpiperidin-1-yl)propan-2-ol;
1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}-4-phenylpiperidin-4-ol;
1-{4-[2-(Methyloxy)phenyl]piperidin-1-yl}-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
N-{1-[(2S)-3-{3-[3-{[2-(4,4-Difluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperidin-4-yl}-2-hydroxyacetamide;
N-[1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-(2-Hydroxy-3-{3-[3-{[2-(4-hydroxypiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
N-(1-[2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)methanesulfonamide;
(3R)-1-[({5-[1-{2-Hydroxy-3-[4-(2-hydroxyphenyl)piperazin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-{4-[2-(Methyloxy)phenyl]piperazin-1-yl}-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1-[2-({5-[1-{2-Hydroxy-3-[4-(pyrimidin-2-yloxy)piperidin-1-yl]propyl}-5-(methylsulfonyl)-4,5,6,7-tetrahydro-1H-pyrazolo[4,3-c]pyridin-3-yl]-2-(trifluoromethyl)phenyl]sulfanyl)ethyl]piperidin-4-ol;
1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
1-[4-(2-Chlorophenyl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
(3R)-1-[({5-[1-{3-[4-(4-Fluorophenyl)piperazin-1-yl]-2-hydroxypropyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-[4-(2-Fluorophenyl)piperazin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
2-[4-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]phenol;
1-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]-4-pyridin-2-ylpiperidin-4-ol;
3-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}phenol;
N-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]methanesulfonamide;
2-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}phenol;
N-{1-[(2S)-2-Hydroxy-3-{5-(methylsulfonyl)-3-[3-[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}acetamide;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(piperidin-1-ylcarbonyl)piperidin-1-yl]propan-2-ol;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrimidin-2-yloxy)piperidin-1-yl]propan-2-ol;
1-[4-(3-Chloropyridin-2-yl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N-[2-({5-[1-{3-[4-(2-Cyanophenyl)piperazin-1-yl]-2-hydroxypropyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(1H-pyrrolo[2,3-b]pyridin-3-yl)piperidin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyrrolidin-1-ylpiperidin-1-yl)propan-2-ol;
(2S)-1-[4-(1-Hydroxy-1-methylethyl)piperidin-1-yl]-3-{5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propan-2-ol;
5-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-2,4-dihydro-3H-1,2,4-triazol-3-one;
(3R)-1-[({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetyl]pyrrolidin-3-ol;
1-[5-(Methylsulfonyl)-3-{3-[(2-morpholin-4-ylethyl)sulfanyl]-4-(trifluoromethyl)phenyl]-4,5,6,7-tetrahydro-1H-pyrazolo[4,3-c]pyridin-1-yl]-3-[4-(piperidin-1-ylcarbonyl)piperidin-1-yl]propan-2-ol;
(2R)-1-[4-(2-Fluorophenyl)piperazin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol;
(4R)-4-Hydroxy-1-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
(4S)-4-Hydroxy-1-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}pyrrolidin-2-one;
1-[4-(3-Chloropyridin-2-yl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
4-Hydroxy-1-(1-[2-hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)pyrrolidin-2-one;
1-[1-(3-{3-[3-{[2-(2,3-Dihydro-1H-inden-2-ylamino)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-[4-(morpholin-4-ylcarbonyl)piperidin-1-yl]propan-2-ol;
2-Hydroxy-N-{1-[(2S)-2-hydroxy-3-{3-[3-{[2-(4-methylpiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl]piperidin-4-yl}-2-methylpropanamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-(4-morpholin-4-ylpiperidin-1-yl)propan-2-ol;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrimidin-2-yloxy)piperidin-1-yl]propan-2-ol;
2-(4-{2-Hydroxy-3-[5-(methylsulfonyl)-3-{3-[(2-oxo-2-piperidin-1-ylethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
4-[1-(2-Hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]benzonitrile;
1-[1-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]-4-hydroxypyrrolidin-2-one;
2-(4-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperazin-1-yl)benzonitrile;
1-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-phenylpiperazin-1-yl)propan-2-ol;
2-{4-[(2R)-3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl]piperazin-1-yl}benzonitrile;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-pyridin-4-ylpiperidin-1-yl)propan-2-ol;
2-[4-(3-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
1-{4-[2-(Ethyloxy)phenyl]piperazin-1-yl}-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
N′-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl]sulfanyl)ethyl]-N,N-dimethylsulfamide;
N-[1-(2-Hydroxy-3-{3-[3-{[2-(2-methyl-3-oxopiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-{4-[2-(Methyloxy)phenyl]piperidin-1-yl}-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-[2-({5-[1-(2-Hydroxy-3-{4-[2-(methyloxy)phenyl]piperidin-1-yl}propyl)-5-(methylsulfonyl)-4,5,6,7-tetrahydro-1H-pyrazolo[4,3-c]pyridin-3-yl]-2-(trifluoromethyl)phenyl}sulfanyl)ethyl]piperidin-4-ol;
N-[1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
4-(2-{[5-{1-[2-Hydroxy-3-(4-hydroxy-4-pyridin-2-ylpiperidin-1-yl)propyl]-5-(methylsulfonyl)-4,5,6,7-tetrahydro-1H-pyrazolo[4,3-c]pyridin-3-yl}-2-(trifluoromethyl)phenyl]sulfanyl]ethyl)-3-methylpiperazin-2-one;
1,1-Dimethylethyl[1-({3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}carbonyl)cyclopropyl]carbamate;
2-[4-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperazin-1-yl]phenol;
1-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
2-[4-(3-{3-[3-{[2-(3,3-Difluoropyrrolidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
(2R)-1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-{4-[2-(methyloxy)phenyl]piperidin-1-yl}propan-2-ol;
N-{1-[2-({5-[5-(Methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl]sulfanyl)ethyl]piperidin-4-yl}acetamide;
N-[1-(2-Hydroxy-3-{3-[3-({2-[2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]methanesulfonamide;
1-(2-Hydroxy-3-{3-[3-(2-[2-(hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}propyl)-4-pyridin-2-ylpiperidin-4-ol;
1-[1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]pyrrolidin-2-one;
N-Cyclopentyl-2-({5-[1-{2-hydroxy-3-[4-(morpholin-4-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)acetamide;
4-[2-({5-[1-{2-Hydroxy-3-[4-(pyrrolidin-1-ylcarbonyl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]-3-methylpiperazin-2-one;
1-[4-(3-Chloropyridin-2-yl)piperazin-1-yl]-3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propan-2-ol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-(4-morpholin-4-ylpiperidin-1-yl)propan-2-ol;
1-{3-[3-({2-[2-(Hydroxymethyl)morpholin-4-yl]ethyl]sulfanyl)-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-{4-[(2-oxo-2-pyrrolidin-1-ylethyl)oxy]piperidin-1-yl}propan-2-ol;
(2R)-1-[4-(3-Chloropyridin-2-yl)piperazin-1-yl]-3-{3-[3-{[2-(4-fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}propan-2-ol;
1-[1-(3-{3-[3-({2-[(1-Methyl-3-phenylpropyl)amino]ethylsulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-{3-[3-{[2-(4-Fluoropiperidin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(morpholin-4-ylcarbonyl)piperidin-1-yl]propan-2-ol;
2-[4-(2-Hydroxy-3-{3-[3-({2-[3-(hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperazin-1-yl]benzonitrile;
2,2,2-Trifluoro-1-{2-[3-{3-[2-(4-fluoro-piperidin-1-yl)-ethylsulfanyl]-4-trifluoromethyl-phenyl}-1-(2-hydroxy-3-piperidin-1-yl-propyl)-1,4,6,7-tetrahydro-pyrazolo[4,3-c]pyridine-5-carbonyl]-pyrrolidin-1-yl}-ethanone;
N-[1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperidin-4-yl]methanesulfonamide;
1-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)-4-pyridin-2-ylpiperidin-4-ol;
2-[4-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]phenol;
1-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-3-[4-(pyrrolidin-1-ylcarbonyl)piperidin-1-yl]propan-2-ol;
(2S)-2-Hydroxy-N-{1-[(2S)-2-hydroxy-3-[5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}propanamide;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(piperidin-1-ylcarbonyl)piperidin-1-yl]propan-2-ol;
1-{3-[3-({2-[3-(Hydroxymethyl)piperidin-1-yl]ethyl}sulfanyl)-4-(trifluoromethyl)phenyl]-5-(methylsulfonyl)-4,5,6,7-tetrahydro-1H-pyrazolo[4,3-c]pyridin-1-yl}-3-[4-(morpholin-4-ylcarbonyl)piperidin-1-yl]propan-2-ol;
1-[1-(3-{5-(Methylsulfonyl)-3-[3-{[2-(3-phenylpiperidin-1-yl)ethyl]sulfanyl}-4-(trifluoromethyl)phenyl]-4,5,6,7-tetrahydro-1H-pyrazolo[4,3-c]pyridin-1-yl}propyl)piperidin-4-yl]pyrrolidin-2-one;
2-[4-(3-{3-[3-{[2-(4-Acetylpiperazin-1-yl)ethyl]sulfanyl}-4-(trifluoromethyl)phenyl]-5-(methylsulfonyl)-4,5,6,7-tetrahydro-1H-pyrazolo[4,3-c]pyridin-1-yl}-2-hydroxypropyl)piperazin-1-yl]benzonitrile;
1-[4-(3,5-Dichloropyridin-4-yl)piperazin-1-yl]-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propan-2-ol;
1,1,1-Trifluoro-N-[2-({5-[1-{2-hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]methanesulfonamide;
1-[4-(3,5-Dichloropyridin-4-yl)piperazin-1-yl]-3-[5-(methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]propan-2-ol;
1,1-Dimethylethyl 4-{2-[1-(2-hydroxy-3-piperidin-1-ylpropyl)-3-{3-[(2-morpholin-4-ylethyl)sulfanyl]-4-(trifluoromethyl)phenyl}-1,4,6,7-tetrahydro-5H-pyrazolo[4,3-c]pyridin-5-yl]-2-oxoethyl}piperidine-1-carboxylate;
1-[1-(3-{3-[3-{[2-(1,4′-Bipiperidin-1′-yl)ethyl]sulfanyl-4-(trifluoromethyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
Methyl {1-[2-hydroxy-3-(4-pyridin-2-ylpiperidin-1-yl)propyl]-3-[3-{[2-({[(phenylmethyl)oxy]carbonyl]amino)ethyl]sulfanyl}-4-(trifluoromethyl)phenyl]-1,4,6,7-tetrahydro-5H-pyrazolo[4,3-c]pyridin-5-yl}(oxo)acetate;
1,1-Dimethylethyl 3-({3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}carbonyl)piperidine-1-carboxylate;
(4S)-4-Hydroxy-1-{1-[(2S)-2-hydroxy-3-[5-(methylsulfonyl)-3-[3-{[2-(octahydroisoquinolin-2(1H)-yl)ethyl]sulfanyl}-4-(trifluoromethyl)phenyl]-4,5,6,7-tetrahydro-1H-pyrazolo[4,3-c]pyridin-1-yl}propyl]piperidin-4-yl}pyrrolidin-2-one;
Ethyl {1-[2-({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl]sulfanyl)ethyl]piperidin-4-yl}carbamate;
N-[2-({5-[1-{2-Hydroxy-3-[4-(2-oxopyrrolidin-1-yl)piperidin-1-yl]propyl}-5-(methylsulfonyl)-4,5,6,7-tetrahydro-1H-pyrazolo[4,3-c]pyridin-3-yl]-2-(trifluoromethyl)phenyl}sulfanyl)ethyl]benzenesulfonamide;
1-[1-(3-{5-(Methylsulfonyl)-3-[3-({2-[4-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-({2-[2-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[1-(3-{5-(Methylsulfonyl)-3-[3-({2-[3-(phenylmethyl)piperidin-1-yl]ethyl}sulfanyl)-4-(trifluoromethyl)phenyl]-4,5,6,7-tetrahydro-1H-pyrazolo[4,3-c]pyridin-1-yl}propyl)piperidin-4-yl]pyrrolidin-2-one;
1-[5-(Methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]-3-{4-[1-(phenylmethyl)-1H-tetrazol-5-yl]piperidin-1-yl}propan-2-ol;
1-[5-(Methylsulfonyl)-3-{3-[(2-morpholin-4-ylethyl)sulfanyl]-4-(trifluoromethyl)phenyl}-4,5,6,7-tetrahydro-1H-pyrazolo[4,3-c]pyridin-1-yl]-3-{4-[1-(phenylmethyl)-1H-tetrazol-5-yl]piperidin-1-yl}propan-2-ol;
1-(2-Hydroxy-3-{5-(methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}propyl)-4-phenylpiperidin-4-ol;
1-(2-Hydroxy-3-{5-(methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}propyl)-4-pyridin-2-ylpiperidin-4-ol;
4-(1-{2-Hydroxy-3-[5-(methylsulfonyl)-3-[3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-2H-1,4-benzoxazin-3(4H)-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-[4-(pyrimidin-2-yloxy)piperidin-1-yl]propan-2-ol;
2-{1-[2-Hydroxy-3-(4-pyridin-2-ylpiperidin-1-yl)propyl]-3-[3-{[2-({[(phenylmethyl)oxy]carbonyl]amino)ethyl]sulfanyl}-4-(trifluoromethyl)phenyl]-1,4,6,7-tetrahydro-5H-pyrazolo[4,3-c]pyridin-5-yl}-2-oxoethyl acetate;
1,1-Dimethylethyl 4-(2-{3-[3-{[2-(4-fluoropiperidin-1-yl)ethyl]sulfanyl}-4-(trifluoromethyl)phenyl]-1-(2-hydroxy-3-piperidin-1-ylpropyl)-1,4,6,7-tetrahydro-5H-pyrazolo[4,3-c]pyridin-5-yl}-2-oxoethyl)piperidine-1-carboxylate;
5-Chloro-1-(1-{2-hydroxy-3-[5-(methylsulfonyl)-3-{3-[(2-piperidin-1-ylethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-benzimidazol-2-one;
Ethyl {1-[({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)acetyl]piperidin-4-yl}carbamate;
5-Chloro-1-methyl-3-(1-{3-[5-(methylsulfonyl)-3-{3-[(2-morpholin-4-yl-2-oxoethyl)sulfanyl]-4-(trifluoromethyl)phenyl}-4,5,6,7-tetrahydro-1H-pyrazolo[4,3-c]pyridin-1-yl]propyl}piperidin-4-yl)-1,3-dihydro-2H-benzimidazol-2-one;
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-[4-(piperidin-1-ylcarbonyl)piperidin-1-yl]propan-2-ol;
1,1-Dimethylethyl {1-[2-({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)ethyl]piperidin-4-yl}carbamate;
1,1-Dimethylethyl {1-[({5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]-2-(trifluoromethyl)phenyl}sulfanyl)acetyl]piperidin-4-yl}carbamate;
5-(Dimethylamino)-3-[1-(2-hydroxy-3-{3-[3-({2-[(3R)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
5-(Dimethylamino)-3-[1-(2-hydroxy-3-{3-[3-({2-[(3S)-3-hydroxypyrrolidin-1-yl]-2-oxoethyl}sulfanyl)-4-(trifluoromethyl)phenyl]-5-(methylsulfonyl)-4,5,6,7-tetrahydro-1H-pyrazolo[4,3-c]pyridin-1-yl}propyl)piperidin-4-yl]-1-methyl-1,3-dihydro-2H-imidazo[4,5-b]pyridin-2-one;
1,1-Dimethylethyl 4-{2-[1-(2-hydroxy-3-{4-[(hydroxyacetyl)amino]piperidin-1-yl}propyl)-3-{3-[(2-pyrrolidin-1-ylethyl)sulfanyl]-4-(trifluoromethyl)phenyl}-1,4,6,7-tetrahydro-5H-pyrazolo[4,3-c]pyridin-5-yl]-2-oxoethyl}piperidine-1-carboxylate; and
1-{5-(Methylsulfonyl)-3-[3-{[2-(4-pyrrolidin-1-ylpiperidin-1-yl)ethyl]sulfanyl}-4-(trifluoromethyl)phenyl]-4,5,6,7-tetrahydro-1H-pyrazolo[4,3-c]pyridin-1-yl}-3-{4-[1-(phenylmethyl)-1H-tetrazol-5-yl]piperidin-1-yl}propan-2-ol;
and pharmaceutically acceptable salts thereof.
